(19) United States
(12) Patent Application Publication
(10) Pub. No.: US 2025/0163066 A1
(43) Pub. Date: May 22, 2025

(54) PROTACs of MALT1

(71) Applicant: TEGID THERAPEUTICS, INC., San Diego, CA (US)

(72) Inventors: Ruben Abagyan, La Jolla, CA (US); Vladislav Zenonovich Parchinsky, Moscow (RU); Alexander Khvat, San Diego, CA (US); Alexandre Vasilievich Ivachtchenko, Hallandale Beach, FL (US); Nikolay Savchuk, San Diego, CA (US)

(21) Appl. No.: 18/840,881

(22) PCT Filed: Feb. 24, 2023
(86) PCT No.: PCT/US2023/013883

§ 371 Date: Aug. 22, 2024

Related U.S. Application Data
(60) Provisional application No. 63/314,205, filed on Feb. 25, 2022.

(51) Int. Cl. C07D 487/04; A61K 31/519; A61K 45/06

(52) U.S. Cl. CPC C07D 487/04; A61K 31/519; A61K 45/06

(57) ABSTRACT

The present invention is directed to the compounds of Formula (I)—PROTACs of MALT1. The PROTACs described herein can be useful in the treatment of diseases or disorders associated with MALT1, such as lymphoma. In particular, the invention is concerned with compounds and pharmaceutical compositions degrade MALT1, methods of treating diseases or disorders associated with MALT1, and methods of synthesizing these compounds.

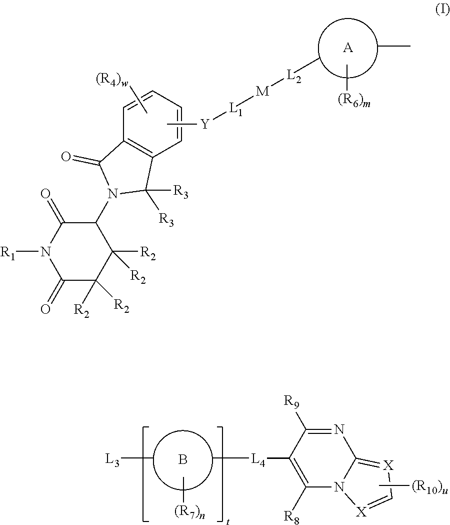

### CROSS-REFERENCE TO RELATED APPLICATIONS

This application claims priority to and the benefit of U.S. Provisional Patent Application Ser. No. 63/314,205 filed Feb. 25, 2022 entitled “PROTACs OF MALT1,” the disclosure of which is incorporated herein by reference in its entirety for all purposes.

### FIELD OF INVENTION

The present invention is directed to PROTACs. The PROTACs described herein can be useful in the treatment of diseases or disorders associated with Mucosa-Associated Lymphoid Tissue Lymphoma Translocation Protein 1 (MALT1), such as Immunodeficiency and Lymphoma. In particular, the invention is concerned with compounds and pharmaceutical compositions that degrades the MALT1, methods of treating diseases or disorders associated with MALT1, and methods of synthesizing these compounds.

### BACKGROUND

Proteolysis targeting chimeric (PROTAC) technology is an effective endogenous protein degradation tool developed in recent years that can ubiquitinate the target proteins through the ubiquitin-proteasome system (UPS) to achieve an effect on tumor growth. A number of literature studies on PROTAC technology have proved an insight into the feasibility of PROTAC technology to degrade target proteins. Additionally, the first oral PROTACs (ARV-110 and ARV-471) have shown encouraging results in clinical trials for prostate and breast cancer treatment, which inspires a greater enthusiasm for PROTAC research (Min Si Qi et al., Front. Pharmacol., 7 May 2021, https://doi.org/10.3389/fphar.2021.692574).

As a traditional treatment method, chemotherapy plays an irreplaceable role in the cancer treatment process. The main disadvantages of traditional anticancer drugs are that most of them have poor selectivity and are easy to develop drug resistance (Mangal et al., Acta Pharmacol. Sin. 2017, 38, 782-797. doi:10.1038/aps.2017.34; Dong et al., Drug Resist. Updates. 2020, 49, 100681. doi:10.1016/j.drup.2020.100681; Yuan et al., Mol. Cancer. 2020, 19, 96. doi:10.1186/s12943-020-01219-0). As a result, the targeted therapy of cancer has attracted people's attention. On this basis, the discoveries of new targets and small molecule inhibitors (SMIs) become powerful treatment strategies. In particular, the development of small molecule kinase inhibitors has become one of the most widely pursued fields in the process of drug discovery and has made great achievements in cancer treatment (Wu et al., Trends Pharmacol. Sci. 2015, 36, 422-439. doi:10.1016/j.tips.2015.04.005). However, after the success, the treatment strategy also faces the same problem of drug resistance as chemotherapy (Xu et al., Front. Cel Dev Biol 2020, 8, 621428. doi:10.3389/fcell.2020.621428). Therefore, drug resistance is the main limitation for cancer therapy and needs to be solved urgently.

In recent years, a novel strategy that targets disease-related proteins for degradation has gained tremendous attention. Proteolysis targeting chimerics (PROTACs), also known as bivalent chemical protein degraders, are heterobifunctional molecules that degrade specific endogenous proteins through the E3 ubiquitin ligase pathway (Potjewyd et al., Cel Chem. Biol. 2020, 27, 47-56. doi:10.1016/j.chembiol.2019.11.006). It structurally connects the protein of interest (POI)-binding ligand and the E3 ubiquitin ligase (E3) ligand through an appropriate linker (Vollmer et al., J. Med Chem. 2020, 63, 1571-162. doi:10.1021/acs.jmedchen 9b00810). The potential advantages of PROTAC technology may compensate for the shortcomings of traditional drug therapy, which promotes its rapid development (Sun and Rao, Biochemistry 2020, 59, 240-249. doi:10.1021/acs.biochem.9b00848).

A bifunctional PROTAC molecule consists of a ligand (mostly small-molecule inhibitor) of the protein of interest (POI) and a covalently linked ligand of an E3 ubiquitin ligase (E3). Upon binding to the POI, the PROTAC can recruit E3 for POI ubiquitination, which is subjected to proteasome-mediated degradation. PROTAC complements nucleic acid-based gene knockdown/out technologies for targeted protein reduction and could mimic pharmacological protein inhibition. To date, PROTACs targeting ˜50 proteins, many of which are clinically validated drug targets, have been successfully developed with several in clinical trials for cancer therapy.

A PROTAC molecule consists of a ligand (mostly small-molecule inhibitor) of the POI and a ligand of an E3 ubiquitin ligase (E3), which are covalently interconnected with a linker of mostly 5-15 carbon or other atoms. Mechanistically, upon binding to POI, the PROTAC can recruit E3 for proximity-induced ubiquitination of POI, which is then subjected to degradation by endogenous 26S proteasome. A recent x-ray structure of POI-PROTAC-E3 ternary complex provides strong evidence to support this mechanism (Gadd M. S. et al., Nat Chem Biol. 2017, 13(5): 514).

The PROTAC technology offers a number of potential advantages (Xin Li et al., J Hematol Oncol 2020, 13, 50. https://doi.org/10.1186/s13045-020-00885-3). First, despite their relatively large molecular weights, PROTACs are more drug-like, which is in contrast to RNA/DNA-based protein reduction agents. By choosing drug-like ligands of POI and E3 followed by medicinal chemistry optimization, PROTACs can have good ADME (absorption, distribution, metabolism, and elimination) properties, which are required to become a clinically useful drug. Second, PROTAC may eliminate the POI sub-stoichiometrically because it can be reused after one round of protein degradation. It is therefore possible that the DC50 of a PROTAC can be significantly lower than its binding affinity (or inhibitory IC50) to the POI. For example, as low as 10 pM of a PROTAC can efficiently induced BRD4 degradation (Qin C. et al., J Med Chem. 2018, 61 (15), 6685-704). This feature provides a potentially huge advantage over pharmacological protein inhibition. Third, since PROTAC could only require a transient binding to the POI, it provides an opportunity to overcome mutation-directed drug resistance. For example, ibrutinib-containing PROTAC MT-802 induced degradation of C481S mutant BTK (which is resistant to ibrutinib) as effectively as the wild-type protein, and potently inhibited proliferation of the ibrutinib-resistant leukemia cells (Buhimschi A. D. et al., Biochemistry 2018, 57 (26), 3564-75). Fourth, PROTAC only requires a ligand that binds to the POI, which may not necessarily affect POI's function. Therefore, PROTAC can possibly target any proteins, including those considered undruggable. Moreover, PROTAC-induced degradation also depends on the lysine residues on the POI surface, which represent additional selectivity requirements. This might lead to a higher selectivity and has been successfully used to develop selective PROTACs targeting an isoform of a protein family, such as CDK9 (Robb C. M. et al., Chem Commun. 2017, 53 (54), 7577-80), BRD4 (Zengerle M. et al., ACS Chem Biol. 2015, 10 (8), 1770-7), MALT1 (Fontan L. et al., Blood 2019, 134 (Supplement 1), 2073-4) and HDAC6 (An Z. et al., Protein & cell 2019, 10 (8), 606-9), starting from a pan-inhibitor of the protein family.

Mucosa-associated lymphoid tissue lymphoma translocation protein 1 (MALT1) is a protein that in humans is encoded by the MALT1 gene. It's the human paracaspase. Genetic ablation of the paracaspase gene in mice and biochemical studies have shown that paracaspase is a crucial protein for T and B lymphocytes activation. It has an important role in the activation of the transcription factor NF-κB, in the production of interleukin-2 (IL-2) and in T and B lymphocytes proliferation. In addition, a role for paracaspase has been shown in the innate immune response mediated by the zymosan receptor Dectin-1 in macrophages and dendritic cells, and in response to the stimulation of certain G protein-coupled receptors. Activation of MALT1 downstream NF-κB signaling and protease activity occurs when BCL10/MALT1 gets recruited to an activated CARD-CC family protein (CARD9, -10, -11 or -14) in a so-called CBM (CARD-CC/BCL10/MALT1) signaling complex. Paracaspase has been shown to have proteolytic activity through its caspase-like domain in T lymphocytes. By targeting paracaspase proteolytic activity, it might be possible to develop new drugs that might be useful for the treatment of certain lymphomas or autoimmune disorders.

In summary, PROTACs targeting ˜50 proteins have been successfully developed to date, among which two compounds are currently in clinical trials to treat therapy-resistant prostate and breast cancer. No clinical outcomes have been disclosed. Given these relatively small numbers of POIs and clinical candidates, it remains to be seen whether these PROTACs can become clinically useful anticancer drugs. However, the PROTAC technology is far from well explored and developed. It has a great potential in the perspective of cancer therapy. There are >600 E3 ubiquitin ligases in human, and many of them may be used for designing a PROTAC (Ottis P. et al, ACS Chem Biol. 2017, 12 (10), 2570-8). Success in this aspect, together with more understanding of the functions and tissue-specific expression of these E3s, could greatly broaden the feasibility, utility, and selectivity of the PROTAC technology.

### SUMMARY

A first aspect of the invention relates to compounds of Formula (A):

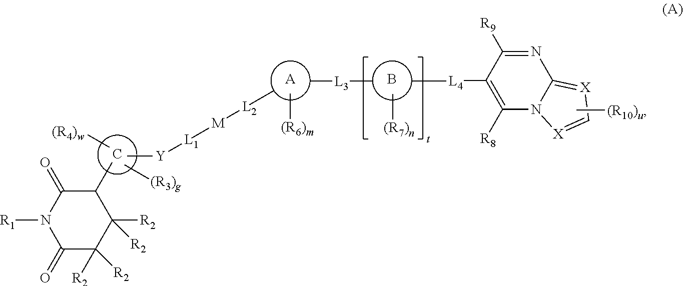

and pharmaceutically acceptable salts, stereoisomers, solvates, prodrugs, or tautomers thereof,
   wherein:
each X is independently selected from N and CH;
Y is selected from

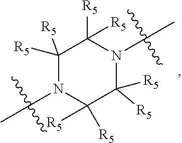

—NR₅C(O)NR₅—, and —NR₅C(O)—;
Ring A is selected from cycloalkyl, aryl, heterocyclyl, and heteroaryl;
Ring B is selected from cycloalkyl, aryl, heterocyclyl, and heteroaryl;
Ring C is selected from cycloalkyl, aryl, heterocyclyl, and heteroaryl;
M is selected from —CH₂—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
L₁ and L₂ are each independently selected from a bond, C₁-C₁₂ alkanediyl, C₂-C₁₂ alkenediyl, C₂-C₁₂ alkynediyl, C₃-C₈ cycloalkanediyl, C₃-C₈ cycloalkanediyl-(CH₂)ₚ—, C₁-C₁₂ alkoxylenyl, —((CH₂)₁₋₆O)ₒ—(CH₂)ₚ—, —((CH₂)₁₋₆O)ₒ—(CH₂)ₚ—NH—, —((CH₂)₁₋₆O)ₒ—(CH₂)ₚ—NH—C(O)—, and —((CH₂)₁₋₆O)ₒ—(CH₂)ₚ—C(O)—NH—; or
M and L₂ together is a bond; or
L₁-M-L₂ is a bond;
L₃ and L₄ are each independently selected from a bond, C₁-C₁₂ alkanediyl, C₂-C₁₂ alkendiyl, C₂-C₁₂ alkyndiyl, C₁-C₁₂ alkoxylenyl, —((CH₂)₁₋₆O)ₒ—(CH₂)ₚ—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
R₁ is selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, —CH₂—OC(O)C₁-C₆ alkyl, —CH₂—O—P(O)(OC₁-C₆ alkyl)₂, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R₁₁;
each R₂, R₃, and R₉ is independently selected from hydrogen, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁; or
two R₃ are ═O;
each R₄, R₆, R₇, and R₁₀ is independently selected from deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁;
each R₅ is independently selected from hydrogen, deuterium, C₁-C₆ alkyl;
R₈ is selected from H, C₁-C₆ alkyl, cycloalkyl, aryl, heterocyclyl, and heteroaryl, wherein the alkyl, cycloalkyl, aryl, heterocycloalkyl, or heteroaryl is optionally substituted with one or more R₁₁; each R₁₁ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl;
each R_{L} is independently selected from hydrogen, deuterium, C₁-C₆ alkyl, C₂-C₆ alkenyl, and C₂-C₆ alkynyl;
R₁₂ and R₁₃ are each independently selected from hydrogen, C₁-C₆ alkyl, C₂-C₆ alkenyl, C₂-C₆ alkynyl, C₁-C₆ haloalkyl, C₁-C₆ hydroxyalkyl, cycloalkyl, aryl, heterocyclyl, and heteroaryl; and
g is an integer selected from 0, 1, and 2;
m and n are integers each independently selected from 0, 1, 2, and 3;
is an integer selected from 0, 1, 2, 3, 4, 5, 6, 7, 8, 9, and 10;
each p is an integer independently selected from 0, 1, 2, 3, 4, 5, 6, 7, 8, 9, and 10;
t is an integer selected from 0 and 1;
u is an integer selected from 0, 1 and 2;
w is an integer selected from 0, 1 and 2;
provided that if t is 0, then L₄ is a bond;
provided that at least one of L₃ and L₄ is not a bond,
   wherein,
cycloalkyl is a saturated or partially unsaturated C₃-C₁₀ monocyclic, C₅-C₁₈ bicyclic (fused, bridged or spiro) or C₆-C₁₈ tricyclic (fused, bridged or spiro) ring system;
aryl is a cyclic, aromatic hydrocarbon ring system comprising 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) comprising 1-7 heteroatoms selected from O, N, S, P, Se, or B;
heteroaryl is a monovalent monocyclic or a polycyclic aromatic ring system comprising 5 to 24 ring atoms, wherein 1-7 ring atoms are heteroatoms selected from N, O, S, P, or B, the remaining ring atoms being C.

Another aspect of the invention relates to compounds of Formula (I):

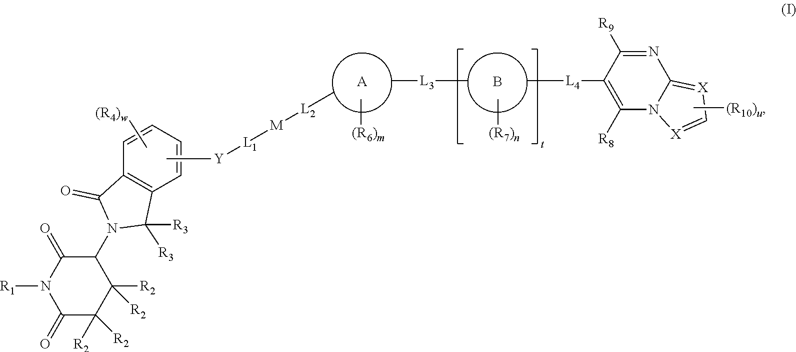

and pharmaceutically acceptable salts, stereoisomers, solvates, prodrugs, or tautomers thereof,
   wherein:
each X is independently selected from N and CH;
Y is selected from

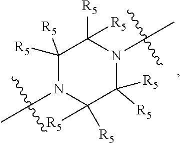

—NR₅C(O)NR₅—, and —NR₅C(O)—;
Ring A is selected from cycloalkyl, aryl, heterocyclyl, and heteroaryl;
Ring B is selected from cycloalkyl, aryl, heterocyclyl, and heteroaryl;
M is selected from —CH₂—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
L₁ and L₂ are each independently selected from a bond, C₁-C₁₂ alkanediyl, C₂-C₁₂ alkenediyl, C₂-C₁₂ alkynediyl, C₃-C₈ cycloalkanediyl, C₃-C₈ cycloalkanediyl-(CH₂)ₚ—, C₁-C₁₂ alkoxylenyl, and —((CH₂)₁₋₆O)ₒ—(CH₂)ₚ—, —((CH₂)₁₋₆O)ₒ—(CH₂)ₚ—NH—, —((CH₂)₁₋₆O)ₒ (CH₂)ₚ—NH—C(O)—, and —((CH₂)₁₋₆O)ₒ—(CH₂)ₚ—C(O)—NH—; or
M and L₂ together is a bond; or
L₁-M-L₂ is a bond;
L₃ and L₄ are each independently selected from a bond, C₁-C₁₂ alkylenyl, C₂-C₁₂ alkenylenyl, C₂-C₁₂ alkynylenyl, C₁-C₁₂ alkoxylenyl, —((CH₂)₁₋₆O)ₒ—(CH₂)ₚ—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
R₁ is selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, —CH₂—OC(O)C₁-C₆ alkyl, —CH₂—O—P(O)(OC₁-C₆ alkyl)₂, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R₁₁;
each R₂, R₃, and R₉ is independently selected from hydrogen, deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁; or
two R₃ form ═O;
each R₄, R₆, R₇, and R₁₀ is independently selected from deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁;
each R₅ is independently selected from hydrogen, deuterium, C₁-C₆ alkyl;
R₈ is selected from H, C₁-C₆ alkyl, cycloalkyl, aryl, heterocyclyl, and heteroaryl, wherein the alkyl, cycloalkyl, aryl, heterocycloalkyl, or heteroaryl is optionally substituted with one or more R₁₁;
each R₁ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl;
each R_{L} is independently selected from hydrogen, deuterium, C₁-C₆ alkyl, C₂-C₆ alkenyl, and C₂-C₆ alkynyl;
R₁₂ and R₁₃ are each independently selected from hydrogen, C₁-C₆ alkyl, C₂-C₆ alkenyl, C₂-C₆ alkynyl, C₁-C₆ haloalkyl, C₁-C₆ hydroxyalkyl, cycloalkyl, aryl, heterocyclyl, and heteroaryl; and
m and n are integers each independently selected from 0, 1, 2, and 3;
is an integer selected from 0, 1, 2, 3, 4, 5, 6, 7, 8, 9, and 10;
each p is an integer independently selected from 0, 1, 2, 3, 4, 5, 6, 7, 8, 9, and 10;
t is an integer selected from 0 and 1;
u is an integer selected from 0, 1 and 2;
w is an integer selected from 0, 1 and 2;
provided that if t is 0, then L₄ is a bond;
provided that at least one of L₃ and L₄ is not a bond,
   wherein,
cycloalkyl is saturated or partially unsaturated C₃-C₁₀ monocyclic, C₅-C₁₈ bicyclic (fused, bridged or spiro) or C₆-C₁₈ tricyclic (fused, bridged or spiro) ring system;
aryl is cyclic, aromatic hydrocarbon groups that have 1 to 3 aromatic rings;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having 1-7 heteroatoms selected from O, N, S, P, Se, or B;
heteroaryl is a monovalent monocyclic or a polycyclic aromatic radical of 5 to 24 ring atoms, containing 1-7 heteroatoms selected from N, O, S, P, or B, the remaining ring atoms being C.

Another aspect of the invention relates to compounds of Formula (II):

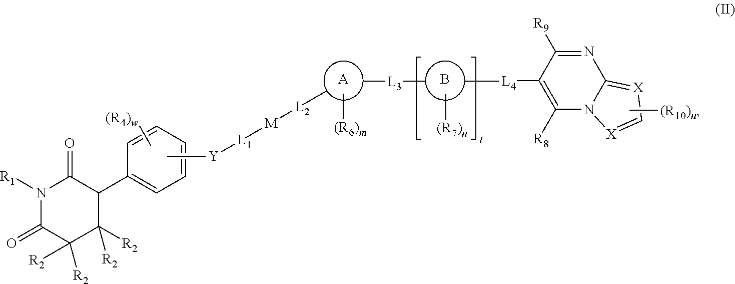

and pharmaceutically acceptable salts, stereoisomers, solvates, prodrugs, or tautomers thereof,
   wherein:
each X is independently selected from N and CH;
Y is selected from

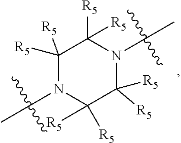

—NR₅C(O)NR₅—, and —NR₅C(O)—;
Ring A is selected from cycloalkyl, aryl, heterocyclyl, and heteroaryl;
Ring B is selected from cycloalkyl, aryl, heterocyclyl, and heteroaryl;
M is selected from —CH₂—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
L₁ and L₂ are each independently selected from a bond, C₁-C₁₂ alkanediyl, C₂-C₁₂ alkenediyl, C₂-C₁₂ alkynediyl, C₃-C₈ cycloalkanediyl, C₃-C₈ cycloalkanediyl-(CH₂)ₚ—, C₁-C₁₂ alkoxylenyl, —((CH₂)₁₋₆O)ₒ—(CH₂)ₚ—, —((CH₂)₁₋₆O)ₒ—(CH₂)ₚ—NH—, —((CH₂)₁₋₆O)ₒ—(CH₂)ₚ—NH—C(O)—, and —((CH₂)₁₋₆O)ₒ—(CH₂)ₚ—C(O)—NH—; or
M and L₂ together is a bond; or
L₁-M-L₂ is a bond;
L₃ and L₄ are each independently selected from a bond, C₁-C₁₂ alkylenyl, C₂-C₁₂ alkendiyl, C₂-C₁₂ alkyndiyl, C₁-C₁₂ alkoxylenyl, —((CH₂)₁₋₆O)ₒ—(CH₂)ₚ—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
R₁ is selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, —CH₂—OC(O)C₁-C₆ alkyl, —CH₂—O—P(O)(OC₁-C₆ alkyl)₂, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R₁₁;
each from R₂ and R₉ is independently selected from hydrogen, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁;
each from R₄, R₆, R₇, and R₁₀ is independently selected from deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁;
each R₅ is independently selected from hydrogen, deuterium, C₁-C₆ alkyl;
R₈ is selected from H, C₁-C₆ alkyl, cycloalkyl, aryl, heterocyclyl, and heteroaryl, wherein the alkyl, cycloalkyl, aryl, heterocycloalkyl, or heteroaryl is optionally substituted with one or more R₁₁;
each R₁₁ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl;
each R_{L} is independently selected from hydrogen, deuterium, C₁-C₆ alkyl, C₂-C₆ alkenyl, and C₂-C₆ alkynyl;
R₁₂ and R₁₃ are each independently selected from hydrogen, C₁-C₆ alkyl, C₂-C₆ alkenyl, C₂-C₆ alkynyl, C₁-C₆ haloalkyl, C₁-C₆ hydroxyalkyl, cycloalkyl, aryl, heterocyclyl, and heteroaryl; and
m and n are integers each independently selected from 0, 1, 2, and 3;
is an integer selected from 0, 1, 2, 3, 4, 5, 6, 7, 8, 9, and 10;
each p is an integer independently selected from 0, 1, 2, 3, 4, 5, 6, 7, 8, 9, and 10;
t is an integer selected from 0 and 1;
u is an integer selected from 0, 1 and 2;
w is an integer selected from 0, 1 and 2;
provided that if t is 0, then L₄ is a bond;
provided that at least one of L₃ and L₄ is not a bond,
   wherein,
cycloalkyl is a saturated or partially unsaturated C₃-C₁₀ monocyclic, C₅-C₁₈ bicyclic (fused, bridged or spiro) or C₆-C₁₈ tricyclic (fused, bridged or spiro) ring system;
aryl is a cyclic, aromatic hydrocarbon ring system comprising 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) comprising 1-7 heteroatoms selected from O, N, S, P, Se, or B;
heteroaryl is a monovalent monocyclic or a polycyclic aromatic ring system comprising 5 to 24 ring atoms, wherein 1-7 ring atoms are heteroatoms selected from N, O, S, P, or B, the remaining ring atoms being C.

Another aspect of the invention is directed to pharmaceutical compositions comprising a compound of Formula (A), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (II), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with MALT1. The method comprises administering to a patient in need of a treatment for diseases or disorders associated with MALT1 an effective amount of a compound of Formula (A), or a pharmaceutically acceptable salt, hydrate, solvate, prodrug, stereoisomer, tautomer, or pharmaceutical composition thereof.

Another aspect of the invention relates to a method of treating a disease or disorder associated with MALT1. The method comprises administering to a patient in need of a treatment for diseases or disorders associated with MALT1 an effective amount of a compound of Formula (I), or a pharmaceutically acceptable salt, hydrate, solvate, prodrug, stereoisomer, tautomer, or pharmaceutical composition thereof.

Another aspect of the invention relates to a method of treating a disease or disorder associated with MALT1. The method comprises administering to a patient in need of a treatment for diseases or disorders associated with MALT1 an effective amount of a compound of Formula (II), or a pharmaceutically acceptable salt, hydrate, solvate, prodrug, stereoisomer, tautomer, or pharmaceutical composition thereof.

Another aspect of the invention is directed to a method of degradation of MALT1. The method involves administering to a patient in need thereof an effective amount of a compound of Formula (A), or a pharmaceutically acceptable salt, hydrate, solvate, prodrug, stereoisomer, tautomer, or pharmaceutical composition thereof.

Another aspect of the invention is directed to a method of degradation of MALT1. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the invention is directed to a method of degradation of MALT1. The method involves administering to a patient in need thereof an effective amount of a compound of Formula (II), or a pharmaceutically acceptable salt, hydrate, solvate, prodrug, stereoisomer, tautomer, or pharmaceutical composition thereof.

Another aspect of the present invention relates to compounds of Formula (A), or pharmaceutically acceptable salts, hydrates, solvates, prodrugs, stereoisomers, tautomers, or pharmaceutical compositions thereof, for use in the manufacture of a medicament for degradation of MALT1.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for degradation of MALT1.

Another aspect of the present invention relates to compounds of Formula (II), or pharmaceutically acceptable salts, hydrates, solvates, prodrugs, stereoisomers, tautomers, or pharmaceutical compositions thereof, for use in the manufacture of a medicament for degradation of MALT1.

Another aspect of the present invention relates to the use of compounds of Formula (A), or pharmaceutically acceptable salts, hydrates, solvates, prodrugs, stereoisomers, tautomers, or pharmaceutical compositions thereof, in the treatment of a disease associated with MALT1.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with MALT1.

Another aspect of the present invention relates to the use of compounds of Formula (II), or pharmaceutically acceptable salts, hydrates, solvates, prodrugs, stereoisomers, tautomers, or pharmaceutical compositions thereof, in the treatment of a disease associated with MALT1.

Another aspect of the present invention relates to compounds of Formula (A), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to compounds of Formula (II), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the invention is directed to a method of treating or preventing a disease or disorder disclosed herein in a subject in need thereof. The method involves administering to a patient in need of the treatment an effective amount of a compound of Formula (A), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (I), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (II), or a pharmaceutically acceptable salt, hydrate, solvate, prodrug, stereoisomer, tautomer, or pharmaceutical composition thereof.

Another aspect of the present invention relates to the use of compounds of Formula (A), or pharmaceutically acceptable salts, hydrates, solvates, prodrugs, stereoisomers, tautomers, or pharmaceutical compositions thereof, in the treatment of a disease or disorder disclosed herein.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder disclosed herein.

Another aspect of the present invention relates to the use of compounds of Formula (II), or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with MALT1, comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating a disease or disorder associated with MALT1, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease or disorder associated with MALT1, comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

The present invention provides PROTACs of MALT1 that are therapeutic agents in the treatment of diseases and disorders.

The present invention further provides compounds and compositions with an improved efficacy and safety profile relative to known PROTACs of MALT1.

The present invention further provides methods of treating a disease or disorder associated with MALT1, comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating a disease or disorder associated with MALT1, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease or disorder associated with MALT1, comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

The present invention provides PROTACs of MALT1 that are therapeutic agents in the treatment of diseases and disorders.

The present invention further provides methods of treating a disease, disorder, or condition selected from Immunodeficiency 12; Lymphoma, Mucosa-Associated Lymphoid Type (MALTOMA); Combined Immunodeficiency; Combined T and B Cell Immunodeficiency; or Lymphoma, comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating a disease, disorder, or condition selected from Immunodeficiency 12; Lymphoma, Mucosa-Associated Lymphoid Type (MALTOMA); Combined Immunodeficiency; Combined T and B Cell Immunodeficiency; or Lymphoma, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease, disorder, or condition selected from Immunodeficiency 12; Lymphoma, Mucosa-Associated Lymphoid Type (MALTOMA); Combined Immunodeficiency; Combined T and B Cell Immunodeficiency; or Lymphoma, comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

In some aspects, the present disclosure provides a compound obtainable by, or obtained by, a method for preparing compounds described herein (e.g., a method comprising one or more steps described in General Procedure).

In some aspects, the present disclosure provides an intermediate as described herein, being suitable for use in a method for preparing a compound as described herein (e.g., the intermediate is selected from the intermediates described in Preparative part—P1-P118).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference in their entireties for all purposes. The references cited herein are not admitted to be prior art to the claimed invention. In the case of conflict, the present specification, including definitions, will control. In addition, the materials, methods, and examples are illustrative only and are not intended to be limiting. In the case of conflict between the chemical structures and names of the compounds disclosed herein, the chemical structures will control.

Other features and advantages of the disclosure will be apparent from the following detailed description and claims

### BRIEF DESCRIPTION OF THE DRAWINGS

FIG. 1 presents bar charts of PROTAC activity against MALT1 for compounds 10, 12 and 13 of the present disclosure, compared to reference compounds Reference 1 and Reference 2, in accordance with experimental conditions presented in Example C, and

FIG. 2 represents bar charts of PROTAC activity against MALT1 for compounds 14, 16 and 17 of the present disclosure, compared to a reference compound Reference 3, in accordance with experimental conditions presented in Example C.

### DETAILED DESCRIPTION

The present disclosure provides methods of treating, preventing, or ameliorating a disease or disorder in which associated with MALT1 by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have one or more substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆)alkyl, and —S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆)alkenyl, —O—(C₂-C₆) alkynyl, (C₂-C₆)alkenyl, (C₂-C₆)alkynyl, —OH, —OP(O)(OH)₂, —OC(O)(C₁-C₆)alkyl, —C(O)(C₁-C₆) alkyl, —OC(O)O(C₁-C₆)alkyl, —NH₂, —NH((C₁-C₆)alkyl), —N((C₁-C₆)alkyl)₂, —S(O)₂—(C₁-C₆) alkyl, —S(O)NH(C₁-C₆)alkyl, and —S(O)N((C₁-C₆)alkyl)₂. The substituents can themselves be optionally substituted. Furthermore, when containing two fused rings the aryl groups herein defined may have one or more saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or a polycyclic aromatic radical of 5 to 24 ring atoms, containing one or more ring heteroatoms selected from N, O, S, P, or B, the remaining ring atoms being C. A polycyclic aromatic radical includes two or more fused rings and may further include two or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B. Heteroaryl as herein defined also means a tricyclic heteroaromatic group containing one or more ring heteroatoms selected from N, O, S, P, or B. Heteroaryl as herein defined also means a tetracyclic heteroaromatic group containing one or more ring heteroatoms selected from N, O, S, P, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo [1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with one or more fully unsaturated aromatic ring. In heteroaryl ring systems containing more than two fused rings, a saturated or partially unsaturated ring may further be fused with a saturated or partially unsaturated ring described herein. Furthermore, when containing three or more fused rings, the heteroaryl groups defined herein may have one or more saturated or partially unsaturated ring spiro-fused. Any saturated or partially unsaturated ring described herein is optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]puyrrolo[1,2-d][1,4]oxazin-9-onyl, or 6a′,7′-dihydro-6′H,9′H-spiro[cyclopropane-1,8′-pyrido[2,3-b]pyrrolo[1,2-d][1,4]oxazin]-9′-onyl.

Halogen or “halo” refers to fluorine, chlorine, bromine, or iodine.

Alkyl refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, t-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

The term “alkoxylenyl” refers to a divalent alkoxy radical. Any of the above mentioned monovalent alkoxy groups may be an alkoxylenyl by abstraction of a second hydrogen atom from the alkyl. Typical alkylene groups include, but are not limited to, —O—CH₂—, —O—CH(CH₃)—, —O—C(CH₃)₂—, —O—CH₂CH₂—, —O—CH₂CH(CH₃)—, —O—CH₂C(CH₃)₂—, —O—CH₂CH₂CH₂—, —O—CH₂CH₂CH₂CH₂—, and the like.

“Cycloalkyl” means mono or polycyclic saturated or partially unsaturated carbon rings containing 3-18 carbon atoms. Polycyclic cycloalkyl may be fused bicyclic cycloalkyl, bridged bicyclic cycloalkyl, or spiro-fused bicyclic cycloalkyl. A polycyclic cycloalkyl comprises at least one non-aromatic ring. Examples of cycloalkyl groups include, without limitations, cyclopropyl, cyclobutyl, cyclopentyl, cyclohexyl, cycloheptanyl, cyclooctanyl, norbornyl, norborenyl, 1,2,3,4-tetrahydronaphthyl, 2,3-dihydro-1H-indenyl, spiro[3.5]nonyl, spiro [5.5]undecyl, bicyclo[1.1.1]pentanyl, bicyclo[2.2.2]octanyl, or bicyclo[2.2.2]octenyl.

“Heterocyclyl”, “heterocycle” or “heterocycloalkyl” mono or polycyclic rings containing 3-24 atoms which include carbon and one or more heteroatoms selected from N, O, S, P, or B and wherein the rings are not aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “aromatic” means a planar ring having 4n+2 electrons in a conjugated system. As used herein, “conjugated system” means a system of connected p-orbitals with delocalized electrons, and the system may include lone electron pairs.

The term “halogenalkyl” as used herein refers to an alkyl group, as defined herein, which is substituted one or more halogen. Examples of halogenalkyl groups include, but are not limited to, trifluoromethyl, difluoromethyl, pentafluoroethyl, trichloromethyl, etc.

The term “halogenalkoxy” as used herein refers to an alkoxy group, as defined herein, which is substituted with one or more halogen. Examples of haloalkyl groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

“Spirocycloalkyl” or “spirocyclyl” means carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom.

The term “spiroheterocycloalkyl”, “spiroheterocycle”, or “spiroheterocyclyl” is understood to mean a spirocycle wherein at least one of the rings is a heterocycle (e.g., at least one of the rings is furanyl, morpholinyl, or piperidinyl).

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The present disclosure also contemplates isotopically-labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound

The term “salt’ refers to pharmaceutically acceptable salts

The term “pharmaceutically acceptable salt” also refers to a salt of the compositions of the present disclosure having an acidic functional group, such as a carboxylic acid functional group, and a base.

“PROTAC of MALT1” as used herein refer to compounds of Formula I and/or compositions comprising a compound of Formula I which degrades of MALT1.

The amount of compound of composition described herein needed for achieving a therapeutic effect may be determined empirically in accordance with conventional procedures for the particular purpose. Generally, for administering therapeutic agents (e.g. compounds or compositions of Formula I (and/or additional agents) described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from cancer provides a therapeutic benefit not only when the underlying condition is eradicated or ameliorated, but also when the subject reports a decrease in the severity or duration of the symptoms associated with the disease, e.g., a decrease in tumor burden, a decrease in circulating tumor cells, an increase in progression free survival. Therapeutic benefit also includes halting or slowing the progression of the underlying disease or disorder, regardless of whether improvement is realized.

### Compounds of the Present Disclosure

In one aspect, the present disclosure provides compounds of Formula (A) and salts, stereoisomers, solvates, prodrugs, isotopic derivatives, and tautomers thereof:

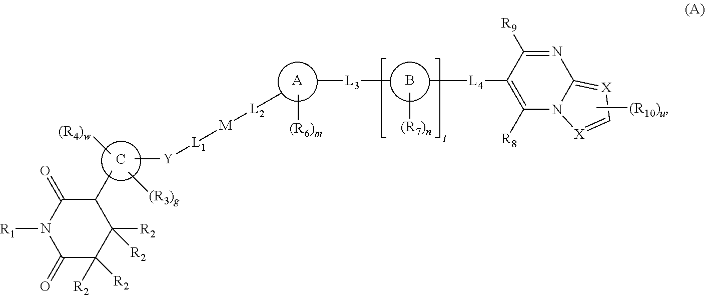

wherein R₁, R₂, R₃, R₄, Y, L₁, M, L₂, A, R₆, L₃, B, R₇, L₄, R₈, R₉, R₁₀, X, g, m, n, t, u, and w are as described herein.

It is understood that, for a compound of Formula (A), R₁, R₂, R₃, R₄, Y, L₁, M, L₂, A, R₆, L₃, B, R₇, L₄, R₈, R₉, R₁₀, X, g, m, n, t, u, and w can each be, where applicable, selected from the groups described herein, and any group described herein for any of R₁, R₂, R₃, R₄, Y, L₁, M, L₂, A, R₆, L₃, B, R₇, L₄, R₈, R₉, R₁₀, X, g, m, n, t, u, and w can be combined, where applicable, with any group described herein for one or more of the remainder of R₁, R₂, R₃, R₄, Y, L₁, M, L₂, A, R₆, L₃, B, R₇, L₄, R₈, R₉, R₁₀, X, g, m, n, t, u, and w.

In some embodiments, the present disclosure provides compounds of Formula (I) and salts, stereoisomers, solvates, prodrugs, isotopic derivatives, and tautomers thereof:

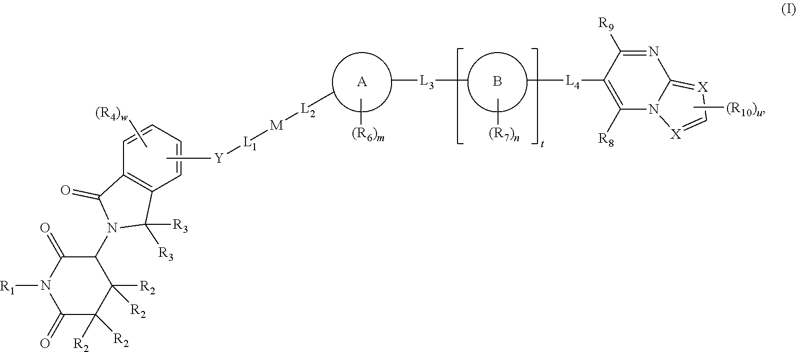

wherein R₁, R₂, R₃, R₄, Y, L₁, M, L₂, A, R₆, L₃, B, R₇, L₄, R₈, R₉, R₁₀, X, m, n, t, u, and w are as described herein.

It is understood that, for a compound of Formula (I), R₁, R₂, R₃, R₄, Y, L₁, M, L₂, A, R₆, L₃, B, R₇, L₄, R₈, R₉, R₁₀, X, m, n, t, u, and w can each be, where applicable, selected from the groups described herein, and any group described herein for any of R₁, R₂, R₃, R₄, Y, L₁, M, L₂, A, R₆, L₃, B, R₇, L₄, R₈, R₉, R₁₀, X, m, n, t, u, and w can be combined, where applicable, with any group described herein for one or more of the remainder of R₁, R₂, R₃, R₄, Y, L₁, M, L₂, A, R₆, L₃, B, R₇, L₄, R₈, R₉, R₁₀, X, m, n, t, u, and w.

In some embodiments, the present disclosure provides compounds of Formula (II) and salts, stereoisomers, solvates, prodrugs, isotopic derivatives, and tautomers thereof:

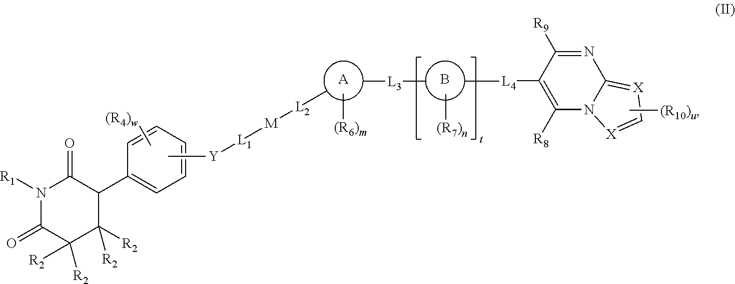

wherein R₁, R₂, R₄, Y, L₁, M, L₂, A, R₆, L₃, B, R₇, L₄, R₈, R₉, R₁₀, X, m, n, t, u, and w are as described herein.

It is understood that, for a compound of Formula (II), R₁, R₂, R₄, Y, L₁, M, L₂, A, R₆, L₃, B, R₇, L₄, R₈, R₉, R₁₀, X, m, n, t, u, and w can each be, where applicable, selected from the groups described herein, and any group described herein for any of R₁, R₂, R₄, Y, L₁, M, L₂, A, R₆, L₃, B, R₇, L₄, R₈, R₉, R₁₀, X, m, n, t, u, and w can be combined, where applicable, with any group described herein for one or more of the remainder of R₁, R₂, R₄, Y, L₁, M, L₂, A, R₆, L₃, B, R₇, L₄, R₈, R₉, R₁₀, X, m, n, t, u, and w.

In some embodiments,
each X is independently selected from N and CH;
Y is selected from

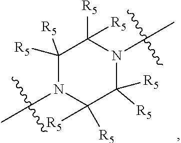

—NR₅C(O)NR₅—, and —NR₅C(O)—;
Ring A is selected from cycloalkyl, aryl, heterocyclyl, and heteroaryl;
Ring B is selected from cycloalkyl, aryl, heterocyclyl, and heteroaryl;
Ring C is selected from cycloalkyl, aryl, heterocyclyl, and heteroaryl;
M is selected from —CH₂—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
L₁ and L₂ are each independently selected from bond, C₁-C₁₂ alkanediyl, C₂-C₁₂ alkenediyl, C₂-C₁₂ alkynediyl, C₃-C₈ cycloalkanediyl, C₃-C₈ cycloalkanediyl-(CH₂)ₚ—, C₁-C₁₂ alkoxylenyl, —((CH₂)₁₋₆O)ₒ—(CH₂)ₚ—, —((CH₂)₁₋₆O)ₒ—(CH₂)ₚ—NH—, —((CH₂)₁₋₆O), —(CH₂)ₚ—NH—C(O)—, and —((CH₂)₁₋₆O)ₒ—(CH₂)ₚ—C(O)—NH—; or
M and L₂ together is a bond; or
L₁-M-L₂ is a bond;
L₃ and L₄ are each independently selected from bond, C₁-C₁₂ alkanediyl, C₂-C₁₂ alkenediyl, C₂-C₁₂ alkynediyl, C₁-C₁₂ alkoxylenyl, —((CH₂)₁₋₆O)ₒ—(CH₂)ₚ—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
R₁ is selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, —CH₂—OC(O)C₁-C₆ alkyl, —CH₂—O—P(O)(OC₁-C₆ alkyl)₂, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R₁₁;
each R₂, R₃, and R₉ is independently selected from hydrogen, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁; or
two R₃ are ═O;
each R₄, R₆, R₇, and R₁₀ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁;
each R₅ is independently selected from hydrogen, C₁-C₆ alkyl;
R₈ is selected from H, C₁-C₆ alkyl, cycloalkyl, aryl, heterocyclyl, and heteroaryl, wherein the alkyl, cycloalkyl, aryl, heterocycloalkyl, or heteroaryl is optionally substituted with one or more R₁₁;
each R₁₁ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl;
each R_{L} is independently selected from hydrogen, deuterium, C₁-C₆ alkyl, C₂-C₆ alkenyl, and C₂-C₆ alkynyl;
R₁₂ and R₁₃ are each independently selected from hydrogen, C₁-C₆ alkyl, C₂-C₆ alkenyl, C₂-C₆ alkynyl, C₁-C₆ haloalkyl, C₁-C₆ hydroxyalkyl, cycloalkyl, aryl, heterocyclyl, and heteroaryl; and
g is an integer selected from 0, 1, and 2;
m and n are integers each independently selected from 0, 1, 2, and 3;
is an integer selected from 0, 1, 2, 3, 4, 5, 6, 7, 8, 9, and 10;
each p is an integer independently selected from 0, 1, 2, 3, 4, 5, 6, 7, 8, 9, and 10;
t is an integer selected from 0 and 1;
u is an integer selected from 0, 1 and 2;
w is an integer selected from 0, 1 and 2;
provided that if t is 0, then L₄ is bond;
provided that at least one of L₃ and L₄ is not bond.

In some embodiments, Ring A is selected from cycloalkyl, aryl, heterocyclyl, and heteroaryl.

In some embodiments, Ring A is cycloalkyl. In some embodiments, Ring A is aryl. In some embodiments, Ring A is heterocyclyl. In some embodiments, Ring A is heteroaryl.

In some embodiments, each X is independently selected from N or CH.

In some embodiments, at least one from two X is nitrogen.

In some embodiments, one X is N, other one X is CH.

In some embodiments, both X are N. In some embodiments both X are CH.

In some embodiments, Ring A is cycloalkyl.

In some embodiments, Ring A is C₃-C₁₂ cycloalkyl.

In some embodiments, Ring A is C₃-C₈ monocyclic cycloalkyl.

In some embodiments, Ring A is

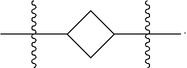 [SMILES: *C1CC(*)C1]

In some embodiments, Ring A is

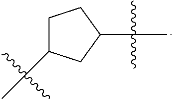 [SMILES: *C1CCC(*)C1]

In some embodiments, Ring A is

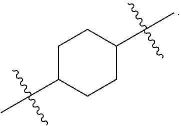 [SMILES: *C1CCC(*)CC1]

In some embodiments, Ring A is

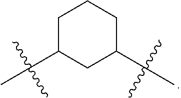 [SMILES: *C1CCCC(*)C1]

In some embodiments, Ring A is fused C₃-C₁₂ bicyclic cycloalkyl. In some embodiments, Ring A is bridged C₃-C₁₂ bicyclic cycloalkyl. In some embodiments, Ring A is spiro-fused C₃-C₁₂ bicyclic cycloalkyl.

In some embodiments, Ring A is aryl.

In some embodiments, Ring A is C₆-C₁₀ aryl.

In some embodiments, Ring A is

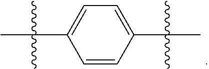 [SMILES: *c1ccc(*)cc1]

In some embodiments, Ring A is

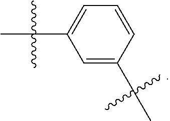 [SMILES: *c1cccc(*)c1]

In some embodiments, Ring A is

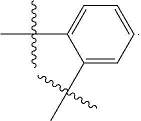 [SMILES: *c1ccccc1*]

In some embodiments, Ring A is

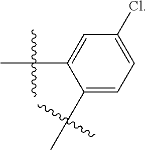 [SMILES: *c1ccc(Cl)cc1*]

In some embodiments, Ring A is heterocyclyl.

In some embodiments Ring A is 3-10 membered heterocyclyl.

In some embodiments, Ring A is 3-membered heterocyclyl.

In some embodiments, Ring A is 3-membered heterocyclyl comprising 1 heteroatom selected from O and N.

In some embodiments, Ring A is 4-membered heterocyclyl.

In some embodiments, Ring A is 4-membered heterocyclyl, comprising 1 or 2 heteroatoms, wherein each heteroatom is independently selected from N, O and S.

In some embodiments, Ring A is 5-membered heterocyclyl.

In some embodiments, Ring A is 5-membered heterocyclyl, comprising 1 or 2 heteroatoms, wherein each heteroatom is independently selected from N, O and S.

In some embodiments, Ring A is 6-membered heterocyclyl.

In some embodiments, Ring A is 6-membered heterocyclyl, comprising 1, 2, or 3 heteroatoms, wherein each heteroatom is independently selected from N, O and S.

In some embodiments, Ring A is 7-membered heterocyclyl.

In some embodiments, Ring A is 7-membered heterocyclyl, comprising 1, 2, or 3 heteroatoms, wherein each heteroatom is independently selected from N, O and S.

In some embodiments, Ring A is

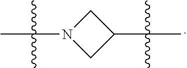 [SMILES: *C1CN(*)C1]

In some embodiments, Ring A is

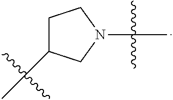 [SMILES: *C1CCN(*)C1]

In some embodiments, Ring A is

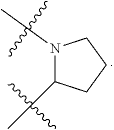 [SMILES: *C1CCCN1*]

In some embodiments, Ring A is

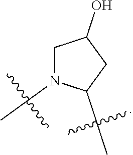 [SMILES: *C1CC(O)CN1*]

In some embodiments, Ring A is

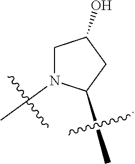

In some embodiments, Ring A is

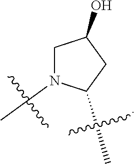

In some embodiments, Ring A is

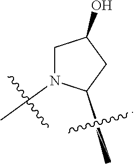

In some embodiments, Ring A is

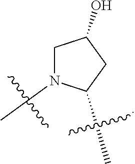

In some embodiments, Ring A is

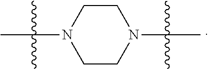 [SMILES: *N1CCN(*)CC1]

In some embodiments, Ring A is

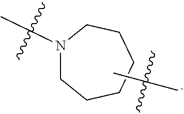

In some embodiments, Ring A is heteroaryl.

In some embodiments, Ring A is 5-membered heteroaryl.

In some embodiments, Ring A is 5-membered heteroaryl, comprising 1, 2, 3 or 4 heteroatoms, wherein each heteroatom is independently selected from N, O, and S.

In some embodiments, Ring A is 6-membered heteroaryl.

In some embodiments, Ring A is 6-membered heteroaryl, comprising 1, 2, 3 or 4 heteroatoms, wherein each heteroatom is independently selected from N, O, and S.

In some embodiments, Ring A is selected from

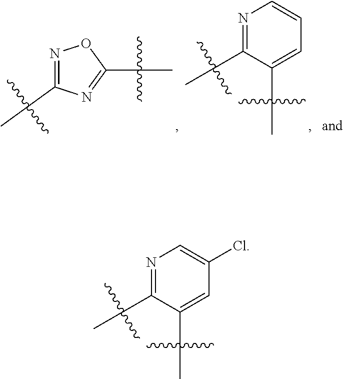 [SMILES: *c1noc(*)n1 ; *c1cccnc1* ; *c1cc(Cl)cnc1*]

In some embodiments, Ring A is

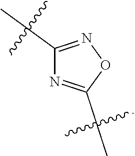 [SMILES: *c1noc(*)n1]

In some embodiments, Ring A is

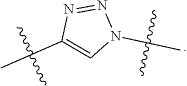 [SMILES: *c1cn(*)nn1]

In some embodiments, Ring A is

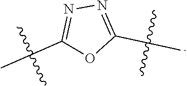 [SMILES: *c1nnc(*)o1]

In some embodiments, Ring A is

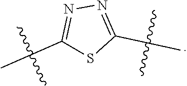 [SMILES: *c1nnc(*)s1]

In some embodiments, Ring A is

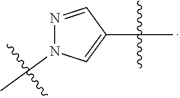 [SMILES: *c1cnn(*)c1]

In some embodiments, Ring A is

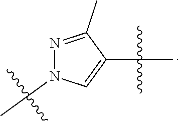 [SMILES: *c1cn(*)nc1C]

In some embodiments, Ring A is

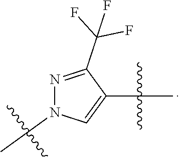 [SMILES: *c1cn(*)nc1C(F)(F)F]

In some embodiments, Ring A is

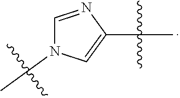 [SMILES: *c1cn(*)cn1]

In some embodiments, Ring A is

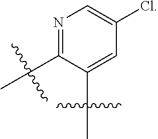 [SMILES: *c1cc(Cl)cnc1*]

In some embodiments, Ring A is

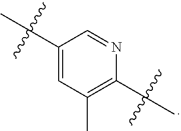 [SMILES: *c1cnc(*)c(C)c1]

In some embodiments, Ring B is selected from cycloalkyl, aryl, heterocyclyl, and heteroaryl.

In some embodiments, Ring B is cycloalkyl. In some embodiments, Ring B is aryl. In some embodiments, Ring B is heterocyclyl. In some embodiments, Ring B is heteroaryl.

In some embodiments, t is 0. In some embodiments, t is 1.

In some embodiments, t is 0.

In some embodiments, t is 1.

In some embodiments, Ring A is cycloalkyl optionally substituted with one or more R₁₁.

In some embodiments, Ring B is cycloalkyl.

In some embodiments, Ring B is C₃-C₁₂ cycloalkyl.

In some embodiments, Ring B is C₃-C₈ monocyclic cycloalkyl.

In some embodiments, Ring B is

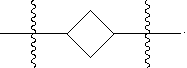 [SMILES: *C1CC(*)C1]

In some embodiments, Ring B is

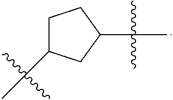 [SMILES: *C1CCC(*)C1]

In some embodiments, Ring B is

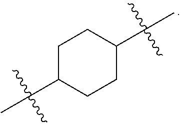 [SMILES: *C1CCC(*)CC1]

In some embodiments, Ring B is

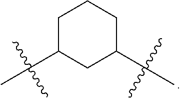 [SMILES: *C1CCCC(*)C1]

In some embodiments, Ring B is fused C₃-C₁₂ bicyclic cycloalkyl. In some embodiments, Ring B is bridged C₃-C₁₂ bicyclic cycloalkyl. In some embodiments, Ring B is spiro-fused C₃-C₁₂ bicyclic cycloalkyl.

In some embodiments, Ring B is aryl.

In some embodiments, Ring B is C₆-C₁₀ aryl.

In some embodiments, Ring B is

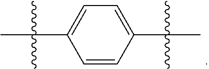 [SMILES: *c1ccc(*)cc1]

In some embodiments, Ring B is

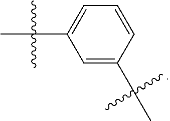 [SMILES: *c1cccc(*)c1]

In some embodiments, Ring B is heterocyclyl.

In some embodiments, Ring B is 3-membered heterocyclyl.

In some embodiments, Ring B is 3-membered heterocyclyl comprising 1 heteroatom selected from O and N.

In some embodiments, Ring B is 4-membered heterocyclyl.

In some embodiments, Ring B is 4-membered heterocyclyl, comprising 1 or 2 heteroatoms, wherein each heteroatom is independently selected from N, O and S.

In some embodiments, Ring B is 5-membered heterocyclyl.

In some embodiments, Ring B is 5-membered heterocyclyl, comprising 1 or 2 heteroatoms, wherein each heteroatom is independently selected from N, O and S.

In some embodiments, Ring B is 6-membered heterocyclyl.

In some embodiments, Ring B is 6-membered heterocyclyl, comprising 1, 2, or 3 heteroatoms, wherein each heteroatom is independently selected from N, O and S.

In some embodiments, Ring B is 7-membered heterocyclyl.

In some embodiments, Ring B is 7-membered heterocyclyl, comprising 1, 2, or 3 heteroatoms, wherein each heteroatom is independently selected from N, O and S.

In some embodiments, Ring B is

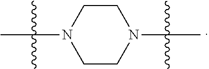 [SMILES: *N1CCN(*)CC1]

In some embodiments, Ring B is

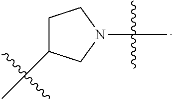 [SMILES: *C1CCN(*)C1]

In some embodiments, Ring B is heteroaryl optionally substituted with one or more R₁₁

In some embodiments, Ring B is heteroaryl.

In some embodiments, Ring B is 5-membered heteroaryl.

In some embodiments, Ring B is 5-membered heteroaryl, comprising 1, 2, 3 or 4 heteroatoms, wherein each heteroatom is independently selected from N, O, and S.

In some embodiments, Ring B is 6-membered heteroaryl.

In some embodiments, Ring B is 6-membered heteroaryl, comprising 1, 2, 3 or 4 heteroatoms, wherein each heteroatom is independently selected from N, O, and S.

In some embodiments, Ring B is

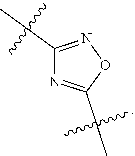 [SMILES: *c1noc(*)n1]

In some embodiments, Ring B is

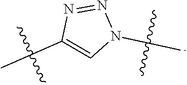 [SMILES: *c1cn(*)nn1]

In some embodiments, Ring B is

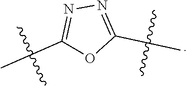 [SMILES: *c1nnc(*)o1]

In some embodiments, Ring B is

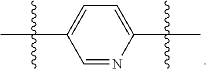 [SMILES: *c1ccc(*)nc1]

In some embodiments, Ring B is

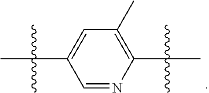 [SMILES: *c1cnc(*)c(C)c1]

In some embodiments, Ring B is

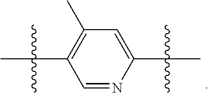 [SMILES: *c1cc(C)c(*)cn1]

In some embodiments, Ring B is

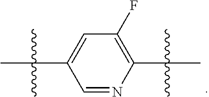 [SMILES: *c1cnc(*)c(F)c1]

In some embodiments, Ring B is

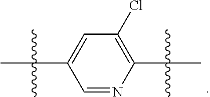 [SMILES: *c1cnc(*)c(Cl)c1]

In some embodiments, Ring B is

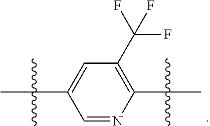 [SMILES: *c1cnc(*)c(C(F)(F)F)c1]

In some embodiments, Ring B is

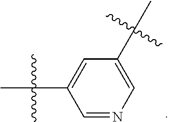 [SMILES: *c1cncc(*)c1]

In some embodiments, Ring B is

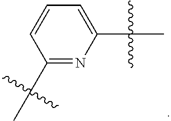 [SMILES: *c1cccc(*)n1]

In some embodiments, Ring C is selected from cycloalkyl, aryl, heterocyclyl, and heteroaryl.

In some embodiments, Ring C is aryl.

In some embodiments, Ring C is

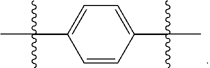 [SMILES: *c1ccc(*)cc1]

In some embodiments, Ring C is

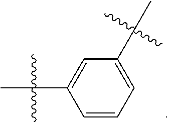 [SMILES: *c1cccc(*)c1]

In some embodiments, Ring C is heterocyclyl.

In some embodiments, Ring C is bicyclic heterocyclyl.

In some embodiments, Ring C is bicyclic heterocyclyl, wherein one ring is C₆ arene.

In some embodiments, Ring C is heteroaryl.

In some embodiments, Ring C is bicyclic heteroaryl.

In some embodiments, Ring C is

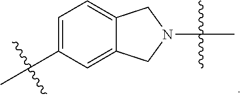 [SMILES: *c1ccc2c(c1)CN(*)C2]

In some embodiments, Ring C is

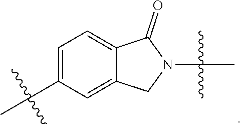 [SMILES: *c1ccc2c(c1)CN(*)C2=O]

In some embodiments, Ring C is

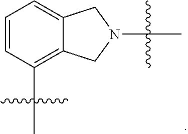 [SMILES: *c1cccc2c1CN(*)C2]

In some embodiments, Ring C is

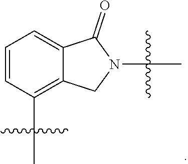 [SMILES: *c1cccc2c1CN(*)C2=O]

In some embodiments, Ring C is

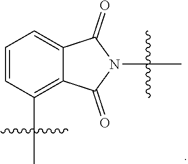 [SMILES: *c1cccc2c1C(=O)N(*)C2=O]

In some embodiments, Ring C is

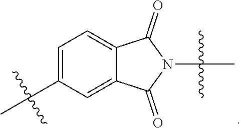 [SMILES: *c1ccc2c(c1)C(=O)N(*)C2=O]

In some embodiments, Ring C is

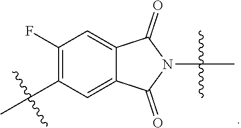 [SMILES: *c1cc2c(cc1F)C(=O)N(*)C2=O]

In some embodiments, Y is selected from

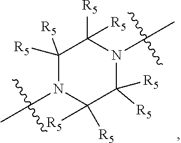

—NR₅C(O)NR₅—, and —NR₅C(O)—.

In some embodiments, Y is

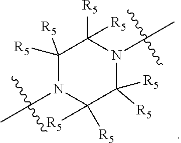

In some embodiments, Y is —NR₅C(O)NR₅—.

In some embodiments, Y is

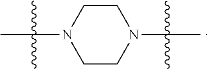 [SMILES: *N1CCN(*)CC1]

In some embodiments, Y is —NHC(O)NH—.

In some embodiments, Y is

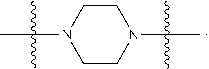 [SMILES: *N1CCN(*)CC1]

In some embodiments, Y is —NHC(O)NH—.

In some embodiments, Y is —NR₅C(O)—.

In some embodiments —NR₅C(O)— links cycle C and L₁ as it shown here: (Ring C)—NR₅C(O)-(L₁).

In some embodiments —NR₅C(O)— links cycle C and L₁ as it shown here: (L₁)-NR₅C(O)-(Ring C).

In some embodiments, Y is —NHC(O)—.

In some embodiments —NHC(O)— links cycle C and L₁ as it shown here: (Ring C)—NHC(O)-(L₁).

In some embodiments —NHC(O)— links cycle C and L₁ as it shown here: (L₁)-NHC(O)-(Ring C).

In some embodiments, M is —CH₂—. In some embodiments, M is —C(O)—. In some embodiments, M is —C(O)NR_{L}—. In some embodiments, M is —C(O)O—. In some embodiments, M is —NR_{L}—. In some embodiments, M is —NR_{L}C(O)—. In some embodiments, M is —NR_{L}C(O)NR_{L}—. In some embodiments, M is —NR_{L}C(O)O—. In some embodiments, M is —NR_{L}SO₂—. In some embodiments, M is —O—. In some embodiments, M is —OC(O)—. In some embodiments, M is —OC(O)NR_{L}—. In some embodiments, M is —OC(O)O—. In some embodiments, M is —S(O)₂NR_{L}—. In some embodiments, M is —S—. In some embodiments, M is —S(O)₂—.

In some embodiments, M is —CH₂—.

In some embodiments, M is —C(O)—.

In some embodiments, M is —O—.

In some embodiments, M is —NR_{L}C(O)—.

In some embodiments, o is 0. In some embodiments, o is 1. In some embodiments, o is 2. In some embodiments, o is 3. In some embodiments, o is 4. In some embodiments, o is 5. In some embodiments, o is 6. In some embodiments, o is 7. In some embodiments, o is 8. In some embodiments, o is 9. In some embodiments, o is 10.

In some embodiments, o is 0.

In some embodiments, o is 1.

In some embodiments, o is 2.

In some embodiments, o is 3.

In some embodiments, each p is an integer independently selected from 0, 1, 2, 3, 4, 5, 6, 7, 8, 9, and 10.

In some embodiments, p is 0. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In some embodiments, p is 0.

In some embodiments, p is 1.

In some embodiments, p is 2.

In some embodiments, p is 3.

In some embodiments, L₁ is bond. In some embodiments, L₁ is C₁-C₁₂ alkanediyl. In some embodiments, L₁ is C₂-C₁₂ alkenediyl. In some embodiments, L₁ is C₂-C₁₂ alkynendiyl. In some embodiments, L₁ is C₃-C₈ cycloalkanediyl. In some embodiments, L₁ is C₃-C₈ cycloalkanediyl-(CH₂)ₚ—. In some embodiments, L₁ is C₁-C₁₂ alkoxylenyl. In some embodiments, L₁ is —((CH₂)₁₋₆O)ₒ—(CH₂)ₚ—. In some embodiments, L₁ is —((CH₂)₁₋₆O)ₒ (CH₂)ₚ—NH—. In some embodiments, L₁ is —((CH₂)₁₋₆O)ₒ—(CH₂)ₚ—NH—C(O)—. In some embodiments, L₁ is —((CH₂)₁₋₆O)ₒ—(CH₂)ₚ—C(O)—NH—.

In some embodiments, L₁ is bond.

In some embodiments, L₁ is C₁-C₁₂ alkylenyl.

In some embodiments, L₁ is —CH₂—.

In some embodiments, L₁ is —CH₂CH₂—.

In some embodiments, L₁ is —CH₂CH₂CH₂—.

In some embodiments, L₁ is —CH₂CH₂CH₂CH₂—.

In some embodiments, L₁ is —CH₂CH₂CH₂CH₂CH₂—.

In some embodiments, L₁ is —((CH₂)₁₋₆O)ₒ—(CH₂)ₚ—.

In some embodiments, L₁ is —CH₂CH₂—O—CH₂CH₂—.

In some embodiments, L₁ is —((CH₂)₁₋₆O)ₒ—(CH₂)ₚ—.

In some embodiments, L₁ is —((CH₂)₁₋₆O)ₒ—(CH₂)ₚ—NH—.

In some embodiments, L₁ is —((CH₂)₁₋₆O)ₒ—(CH₂)ₚ—NH—C(O)—.

In some embodiments, L₁ is —((CH₂)₁₋₆O))—(CH₂)ₚ—C(O)—NH—.

In some embodiments, L₁ is C₃-C₈ cycloalkanediyl.

In some embodiments, L₁ is

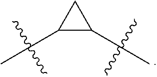 [SMILES: *C1CC1*]

In some embodiments, L₁ is

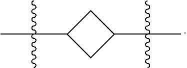 [SMILES: *C1CC(*)C1]

In some embodiments, L₁ is

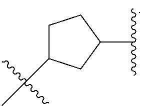 [SMILES: *C1CCC(*)C1]

In some embodiments, L₁ is

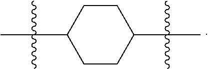 [SMILES: *C1CCC(*)CC1]

In some embodiments, L₁ is

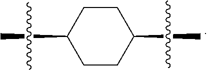

In some embodiments, L₁ is

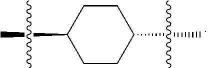

In some embodiments, L₁ is

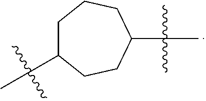 [SMILES: *C1CCCC(*)CC1]

In some embodiments, L₁ is

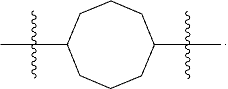 [SMILES: *C1CCCC(*)CCC1]

In some embodiments, L₂ is bond. In some embodiments, L₂ is C₁-C₁₂ alkanediyl. In some embodiments, L₂ is C₂-C₁₂ alkenediyl. In some embodiments, L₂ is C₂-C₁₂ alkynediyl. In some embodiments, L₂ is C₃-C₈ cycloalkanediyl. In some embodiments, L₂ is C₃-C₈ cycloalkanediyl-(CH₂)ₚ—. In some embodiments, L₂ is C₁-C₁₂ alkoxylenyl. In some embodiments, L₂ is —((CH₂)₁₋₆O)ₒ—(CH₂)ₚ—. In some embodiments, L₂ is —((CH₂)₁₋₆O)ₒ (CH₂)ₚ—NH—. In some embodiments, L₂ is —((CH₂)₁₋₆O)ₒ—(CH₂)ₚ—NH—C(O)—. In some embodiments, L₂ is —((CH₂)₁₋₆O)ₒ—(CH₂)ₚ—C(O)—NH—.

In some embodiments, L₂ is bond.

In some embodiments, L₂ is C₁-C₁₂ alkylenyl.

In some embodiments, L₂ is —CH₂—.

In some embodiments, L₂ is —CH₂CH₂—.

In some embodiments, L₂ is —CH₂CH₂CH₂—.

In some embodiments, L₂ is —CH₂CH₂CH₂CH₂—.

In some embodiments, L₂ is —CH₂CH₂CH₂CH₂CH₂—.

In some embodiments, L₂ is —CH₂—C(CH₃)₂—CH₂—.

In some embodiments, L₂ is —((CH₂)₁₋₆O)ₒ—(CH₂)ₚ—.

In some embodiments, L₂ is —CH₂—O—CH₂CH₂CH₂—.

In some embodiments, L₂ is —CH₂CH₂CH₂CH₂—O—CH₂—.

In some embodiments, L₂ is —((CH₂)₁₋₆O)ₒ—(CH₂)ₚ—.

In some embodiments, L₂ is —((CH₂)₁₋₆O)ₒ—(CH₂)ₚ—NH—.

In some embodiments, L₂ is —((CH₂)₁₋₆O)ₒ—(CH₂)ₚ—NH—C(O)—.

In some embodiments, L₂ is —((CH₂)₁₋₆O)ₒ—(CH₂)ₚ—C(O)—NH—.

In some embodiments, L₂ is C₃-C₈ cycloalkanediyl-(CH₂)ₚ—.

In some embodiments, L₂ is C₃-C₈ cycloalkanediyl-CH₂—.

In some embodiments, L₂ is

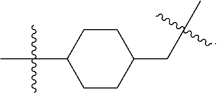 [SMILES: *CC1CCC(*)CC1]

In some embodiments, L₂ is C₃-C₈ cycloalkanediyl.

In some embodiments, L₂ is

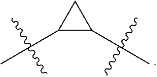 [SMILES: *C1CC1*]

In some embodiments, L₂ is

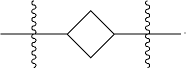 [SMILES: *C1CC(*)C1]

In some embodiments, L₂ is

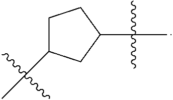 [SMILES: *C1CCC(*)C1]

In some embodiments, L₂ is

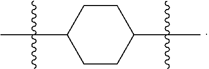 [SMILES: *C1CCC(*)CC1]

In some embodiments, L₂ is

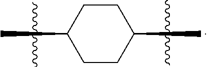

In some embodiments, L₂ is

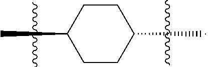

In some embodiments, L₂ is

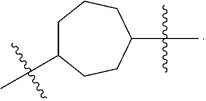 [SMILES: *C1CCCC(*)CC1]

In some embodiments, L₂ is

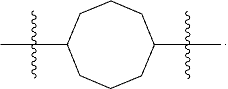 [SMILES: *C1CCCC(*)CCC1]

In some embodiments, M-L₂ is a bond.

In some embodiments, L₁-M-L₂ is a bond.

In some embodiments, L₃ is bond. In some embodiments, L₃ is C₁-C₁₂ alkanediyl. In some embodiments, L₃ is C₂-C₁₂ alkenediyl. In some embodiments, L₃ is C₂-C₁₂ alkynediyl. In some embodiments, L₃ is C₁-C₁₂ alkoxylenyl. In some embodiments, L₃ is —((CH₂)₁₋₆O)ₒ—(CH₂)ₚ—. In some embodiments, L₃ is —C(O)—. In some embodiments, L₃ is —C(O)NR_{L}—. In some embodiments, L₃ is —C(O)O—. In some embodiments, L₃ is —NR_{L}—. In some embodiments, L₃ is —NR_{L}C(O)—. In some embodiments, L₃ is —NR_{L}C(O)NR_{L}—. In some embodiments, L₃ is —NR_{L}C(O)O—. In some embodiments, L₃ is —NR_{L}SO₂—. In some embodiments, L₃ is —O—. In some embodiments, L₃ is —OC(O)—. In some embodiments, L₃ is —OC(O)NR_{L}—. In some embodiments, L₃ is —OC(O)O—. In some embodiments, L₃ is —S(O)₂NR_{L}—. In some embodiments, L₃ is —S—. In some embodiments, L₃ is —S(O)₂—.

In some embodiments, L₃ is bond.

In some embodiments, L₄ is bond. In some embodiments, L₄ is C₁-C₁₂ alkanediyl. In some embodiments, L₄ is C₂-C₁₂ alkenediyl. In some embodiments, L₄ is C₂-C₁₂ alkynediyl. In some embodiments, L₄ is C₁-C₁₂ alkoxylenyl. In some embodiments, L₄ is —((CH₂)₁₋₆O)ₒ—(CH₂)ₚ—. In some embodiments, L₄ is —C(O)—. In some embodiments, L₄ is —C(O)NR_{L}—. In some embodiments, L₄ is —C(O)O—. In some embodiments, L₄ is —NR_{L}—. In some embodiments, L₄ is —NR_{L}C(O)—. In some embodiments, L₄ is —NR_{L}C(O)NR_{L}—. In some embodiments, L₄ is —NR_{L}C(O)O—. In some embodiments, L₄ is —NR_{L}SO₂—. In some embodiments, L₄ is —O—. In some embodiments, L₄ is —OC(O)—. In some embodiments, L₄ is —OC(O)NR_{L}—. In some embodiments, L₄ is —OC(O)O—. In some embodiments, L₄ is —S(O)₂NR_{L}—. In some embodiments, L₄ is —S—. In some embodiments, L₄ is —S(O)₂—.

In some embodiments, L₄ is —NR_{L}C(O)NR_{L}—.

In some embodiments, L₄ is —NHC(O)NH—.

In some embodiments, L₄ is —NR_{L}C(O)—.

In some embodiments, L₄ is —NHC(O)—.

In some embodiments, L₄ is —C(O)NH—.

In some embodiments, R₁ is selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, —CH₂—OC(O)C₁-C₆ alkyl, —CH₂—O—P(O)(OC₁-C₆ alkyl)₂, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R₁₁.

In some embodiments, R₁ is hydrogen. In some embodiments, R₁ is deuterium. In some embodiments, R₁ is C₁-C₆ alkyl. In some embodiments, R₁ is C₂-C₆ alkenyl. In some embodiments, R₁ is C₂-C₆ alkynyl. In some embodiments, R₁ is C₁-C₆ haloalkyl. In some embodiments, R₁ is C₁-C₆ alkoxy. In some embodiments, R₁ is C₁-C₆ haloalkoxy. In some embodiments, R₁ is cycloalkyl. In some embodiments, R₁ is aryl. In some embodiments, R₁ is heterocyclyl.

In some embodiments, R₁ is hydrogen.

In some embodiments, R₁ is C₁-C₆ alkyl.

In some embodiments, R₁ is methyl.

In some embodiments, R₁ is —CH₂—OC(O)C₁-C₆ alkyl.

In some embodiments, R₁ is

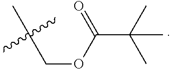 [SMILES: *COC(=O)C(C)(C)C]

In some embodiments, R₁ is —CH₂—O—P(O)(OC₁-C₆ alkyl)₂.

In some embodiments, R₁ is

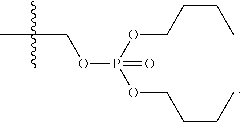 [SMILES: *COP(=O)(OCCCC)OCCCC]

In some embodiments, each R₂ is independently selected from hydrogen, deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁.

In some embodiments, each R₂ is H.

In some embodiments, at least one R₂ is halogen.

In some embodiments, at least one R₂ is methyl.

In some embodiments, at least one R₂ is methoxy.

In some embodiments, at least one R₂ is —CN.

In some embodiments, each R₃ is independently selected from hydrogen, deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁.

In some embodiments, each R₃ is H.

In some embodiments, at least one R₃ is halogen.

In some embodiments, one R₃ is halogen, other one is H.

In some embodiments, at least one R₃ is methyl.

In some embodiments, each R₃ is methyl.

In some embodiments, one R₃ is methyl, other one is H.

In some embodiments, two R₃ form ═O.

In some embodiments, each R₄ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁.

In some embodiments, w is an integer selected from 0, 1 and 2.

In some embodiments, w is 0. In some embodiments, w is 1. In some embodiments, w is 2.

In some embodiments, w is 0.

In some embodiments, R₄ is halogen.

In some embodiments, R₄ is F.

In some embodiments, w is 1 and R₄ is halogen.

In some embodiments, w is 1 and R₄ is F. In some embodiments, w is 1 and R₄ is Cl. In some embodiments, w is 1 and R₄ is Br. In some embodiments, w is 1 and R₄ is I.

In some embodiments, w is 1 and R₄ is F.

In some embodiments, w is 1 and R₄ is —CN.

In some embodiments, w is 1 and R₄ is —OH.

In some embodiments, w is 1 and R₄ is methyl.

In some embodiments, w is 1 and R₄ is methoxy.

In some embodiments, each R₅ is independently selected from hydrogen, C₁-C₆ alkyl.

In some embodiments, each R₅ is H.

In some embodiments, each R₆ is independently selected from deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁.

In some embodiments, m is an integer selected from 0, 1, 2, and 3.

In some embodiments, m is 0.

In some embodiments, m is 1 and R₆ is halogen.

In some embodiments, m is 1 and R₆ is F. In some embodiments, m is 1 and R₆ is C₁. In some embodiments, m is 1 and R₆ is Br. In some embodiments, m is 1 and R₆ is I.

In some embodiments, m is 1 and R₆ is —OH.

In some embodiments, m is 1 and R₆ is —CN.

In some embodiments, m is 1 and R₆ is C₁-C₆ alkyl.

In some embodiments. m is 1 and R₆ is —NR₁₂R₁₃.

In some embodiments, m is 2.

In some embodiments, m is 3.

In some embodiments, each R₇ is independently selected from deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁.

In some embodiments, n is an integer selected from 0, 1, 2, and 3.

In some embodiments, n is 0.

In some embodiments, n is 1 and R₇ is halogen.

In some embodiments, n is 1 and R₇ is Cl.

In some embodiments, n is 1 and R₇ is C₁-C₆ alkyl.

In some embodiments, n is 1 and R₇ is CH₃.

In some embodiments, n is 2.

In some embodiments, n is 3.

In some embodiments, R₈ is selected from H, C₁-C₆ alkyl, cycloalkyl, aryl, heterocyclyl, and heteroaryl, wherein the alkyl, cycloalkyl, aryl, heterocycloalkyl, or heteroaryl is optionally substituted with one or more R₁₁.

In some embodiments, R₈ is H.

In some embodiments, R₈ is C₁-C₆ alkyl.

In some embodiments, R₈ is C₁-C₆ alkyl optionally substituted with one or more R₁₁.

In some embodiments, R₈ is methyl.

In some embodiments, R₈ is ethyl.

In some embodiments, R₈ is propyl.

In some embodiments, R₈ is n-propyl.

In some embodiments, R₈ is iso-propyl.

In some embodiments, R₈ is butyl.

In some embodiments, R₈ is tert-butyl.

In some embodiments, R₈ is

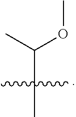 [SMILES: *C(C)OC]

In some embodiments, R₈ is

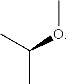

In some embodiments, R₈ is cycloalkyl.

In some embodiments, Ring R₈ is C₃-C₁₂ cycloalkyl.

In some embodiments, Ring R₈ is C₃-C₈ monocyclic cycloalkyl.

In some embodiments, R₈ is 3-membered cycloalkyl. In some embodiments, R₈ is 4-membered cycloalkyl. In some embodiments, R₈ is 5-membered cycloalkyl. In some embodiments, R₈ is 6-membered cycloalkyl. In some embodiments, R₈ is 7 membered cycloalkyl.

In some embodiments, R₈ is

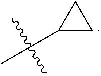 [SMILES: *C1CC1]

In some embodiments, R₈ is

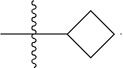 [SMILES: *C1CCC1]

In some embodiments, R₈ is

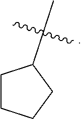 [SMILES: *C1CCCC1]

In some embodiments, R₈ is

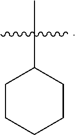 [SMILES: *C1CCCCC1]

In some embodiments, Ring R₈ is fused C₅-C₁₂ bicyclic cycloalkyl. In some embodiments, Ring R₈ is bridged C₅-C₁₂ bicyclic cycloalkyl. In some embodiments, Ring R₈ is spiro-fused C₅-C₁₂ bicyclic cycloalkyl.

In some embodiments, R₈ is aryl.

In some embodiments, Ring R₈ is C₆-C₁₀ aryl.

In some embodiments, R₈ is phenyl.

In some embodiments, R₈ is

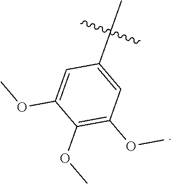

In some embodiments, R₈ is heterocyclyl.

In some embodiments, R₈ is 3-10 membered heterocyclyl.

In some embodiments, R₈ is 3-membered heterocyclyl. In some embodiments, R₈ is 4-membered heterocyclyl. In some embodiments, R₈ is 5-membered heterocyclyl. In some embodiments, R₈ is 6-membered heterocyclyl. In some embodiments, R₈ is 7-membered heterocyclyl.

In some embodiments, R₈ is 3-membered heterocyclyl.

In some embodiments, R₈ is 3-membered heterocyclyl comprising 1 heteroatom selected from O and N.

In some embodiments, R₈ is 4-membered heterocyclyl.

In some embodiments, R₈ is 4-membered heterocyclyl, comprising 1 or 2 heteroatoms, wherein each heteroatom is independently selected from N, O and S.

In some embodiments, R₈ is 5-membered heterocyclyl.

In some embodiments, R₈ is 5-membered heterocyclyl, comprising 1 or 2 heteroatoms, wherein each heteroatom is independently selected from N, O and S.

In some embodiments, R₈ is 6-membered heterocyclyl.

In some embodiments, R₈ is 6-membered heterocyclyl, comprising 1, 2, or 3 heteroatoms, wherein each heteroatom is independently selected from N, O and S.

In some embodiments, R₈ is 7-membered heterocyclyl.

In some embodiments, R₈ is 7-membered heterocyclyl, comprising 1, 2, or 3 heteroatoms, wherein each heteroatom is independently selected from N, O and S.

In some embodiments, R₈ is

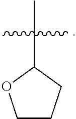 [SMILES: *C1CCCO1]

In some embodiments, R₈ is

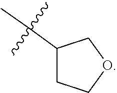 [SMILES: *C1CCOC1]

In some embodiments, R₈ is

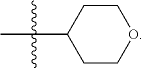 [SMILES: *C1CCOCC1]

In some embodiments, R₈ is heteroaryl.

In some embodiments, R₈ is 5-membered heteroaryl. In some embodiments, R₈ is 6-membered heteroaryl.

In some embodiments, R₈ is

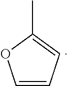

In some embodiments, R₉ is selected from hydrogen, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁.

In some embodiments, R₉ is hydrogen.

In some embodiments, R₉ is cycloalkyl.

In some embodiments, R₉ is C₃-C₈ cycloalkyl.

In some embodiments, R₉ is

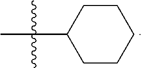 [SMILES: *C1CCCCC1]

In some embodiments, R₁₀ is selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁.

In some embodiments, u is 0. In some embodiments, u is 1.

In some embodiments, u is 1.

In some embodiments, R₁₀ is halogen.

In some embodiments, R₁₀ is F.

In some embodiments, R₁₀ is Cl.

In some embodiments, R₁₀ is Br.

In some embodiments, R₁₀ is C₁-C₆ alkyl.

In some embodiments, R₁₀ is —CH₃.

In some embodiments, each R₁ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl.

In some embodiments, R₁₁ is halogen.

In some embodiments, R₁ is F. In some embodiments, R₁ is C₁. In some embodiments, R₁ is Br.

In some embodiments, R₁ is —OH.

In some embodiments, R₁ is —CN.

In some embodiments, R₁ is C₁-C₆ alkyl.

In some embodiments, R₁ is —CF₃.

In some embodiments, each R_{L} is independently selected from hydrogen, deuterium, C₁-C₆ alkyl, C₂-C₆ alkenyl, and C₂-C₆ alkynyl.

In some embodiments, R_{L} is hydrogen.

In some embodiments, R_{L} is C₁-C₆ alkyl.

In some embodiments, R₁₂ and R₁₃ are each independently selected from hydrogen, C₁-C₆ alkyl, C₂-C₆ alkenyl, C₂-C₆ alkynyl, C₁-C₆ haloalkyl, C₁-C₆ hydroxyalkyl, cycloalkyl, aryl, heterocyclyl, and heteroaryl.

In some embodiments, R₁₂ and R₁₃ are each hydrogen.

In some embodiments, R₁₂ is methyl and R₁₃ is H.

In some embodiments, R₁₂ is H and R₁₃ is methyl.

In some embodiments, R₁₂ and R₁₃ are each methyl.

In some embodiments, R₁₂ and R₁₃ are each ethyl.

In some embodiments, R₁₂ is H and R₁₃ is —OH.

In some embodiments, the compound is of Formula (I-I):

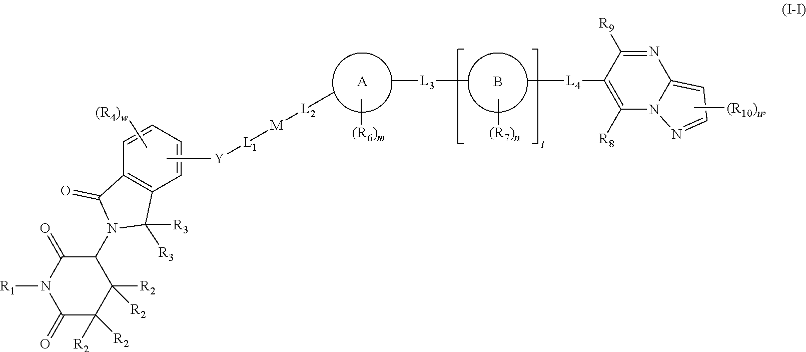

or a pharmaceutically acceptable salt, stereoisomer, solvate, prodrug, isotopic derivative, or tautomer thereof wherein all variables are as defined herein.

In some embodiments, the compound is of Formula (I-II):

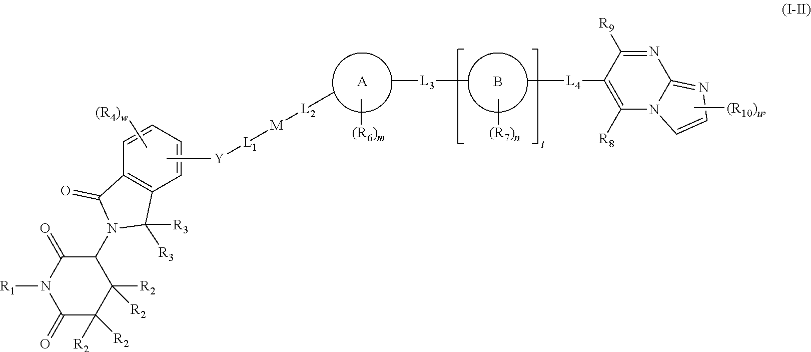

or a pharmaceutically acceptable salt, stereoisomer, solvate, prodrug, isotopic derivative, or tautomer thereof wherein all variables are as defined herein.

In some embodiments, the compound is of Formula (I-III):

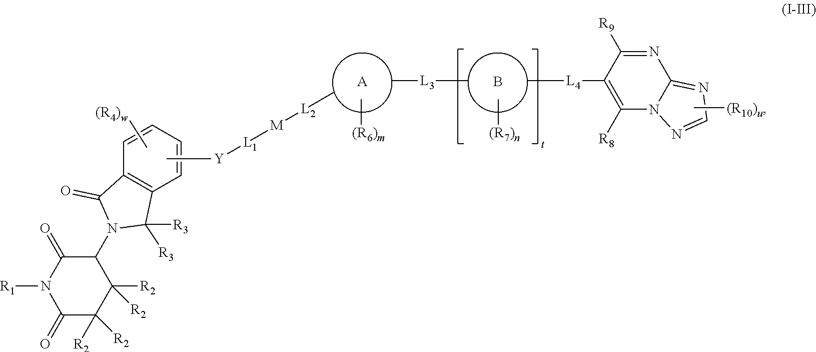

or a pharmaceutically acceptable salt, stereoisomer, solvate, prodrug, isotopic derivative, or tautomer thereof wherein all variables are as defined herein.

In some embodiments, the compound is of Formula (I-I-H):

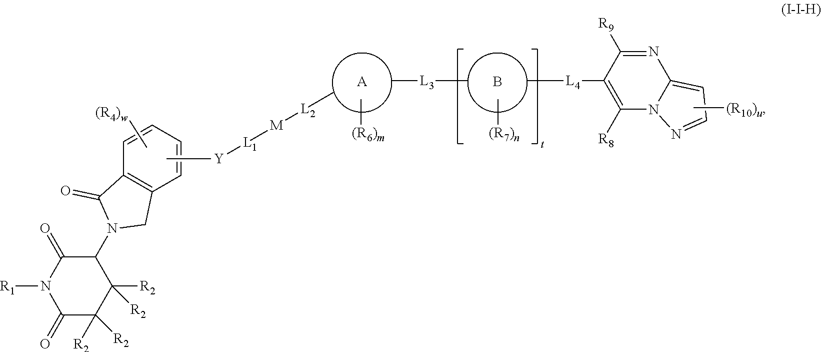

or a pharmaceutically acceptable salt, stereoisomer, solvate, prodrug, isotopic derivative, or tautomer thereof wherein all variables are as defined herein.

In some embodiments, the compound is of Formula (I-II-H):

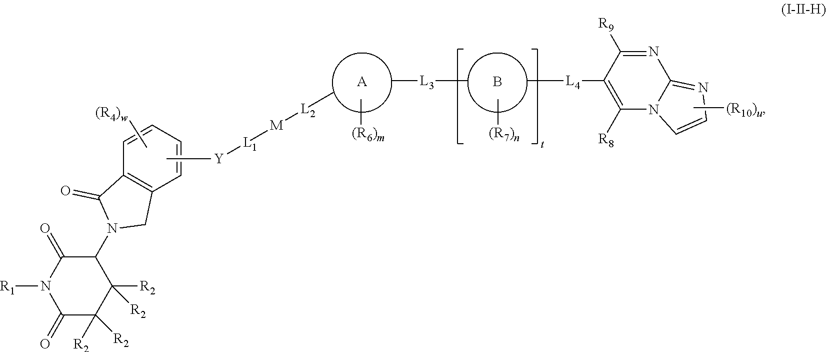

or a pharmaceutically acceptable salt, stereoisomer, solvate, prodrug, isotopic derivative, or tautomer thereof wherein all variables are as defined herein.

In some embodiments, the compound is of Formula (I-I-O):

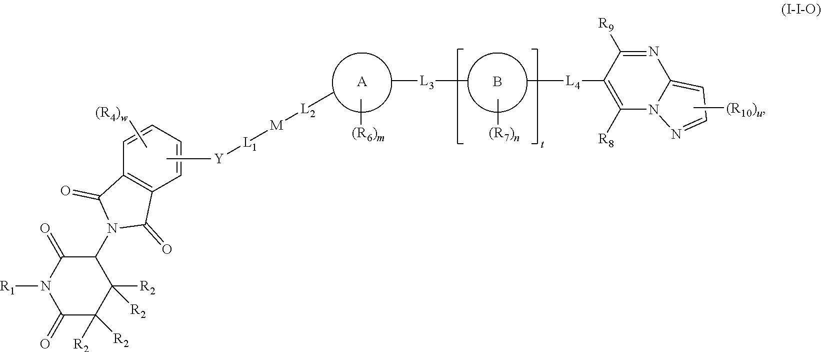

or a pharmaceutically acceptable salt, stereoisomer, solvate, prodrug, isotopic derivative, or tautomer thereof wherein all variables are as defined herein.

In some embodiments, the compound is of Formula (I-II-O):

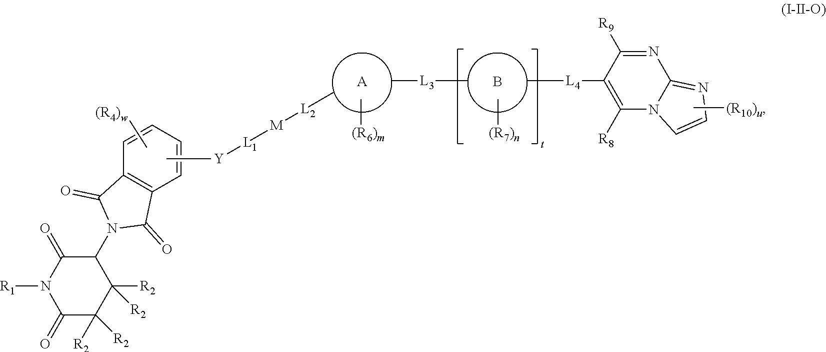

or a pharmaceutically acceptable salt, stereoisomer, solvate, prodrug, isotopic derivative, or tautomer thereof wherein all variables are as defined herein.

In some embodiments, the compound is of Formula (I-I′):

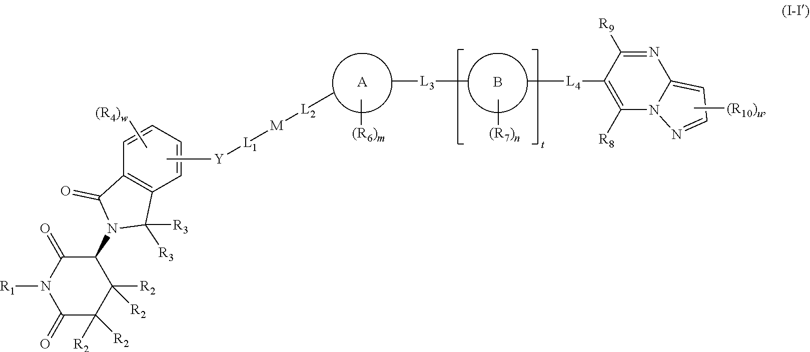

or a pharmaceutically acceptable salt, stereoisomer, solvate, prodrug, isotopic derivative, or tautomer thereof wherein all variables are as defined herein.

In some embodiments, the compound is of Formula (I-II′):

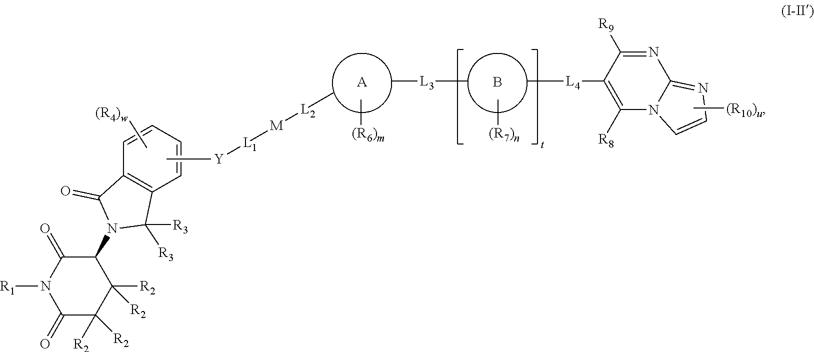

or a pharmaceutically acceptable salt, stereoisomer, solvate, prodrug, isotopic derivative, or tautomer thereof wherein all variables are as defined herein.

In some embodiments, the compound is of Formula (I-I-A):

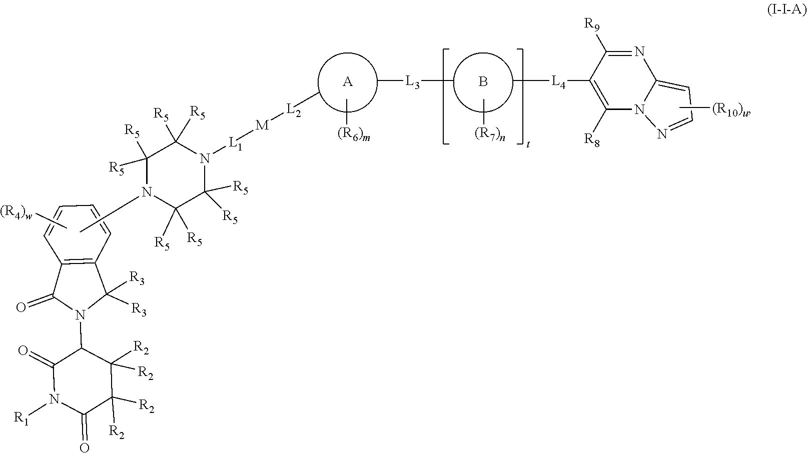

or a pharmaceutically acceptable salt, stereoisomer, solvate, prodrug, isotopic derivative, or tautomer thereof wherein all variables are as defined herein.

In some embodiments, the compound is of Formula (I-I-A-1):

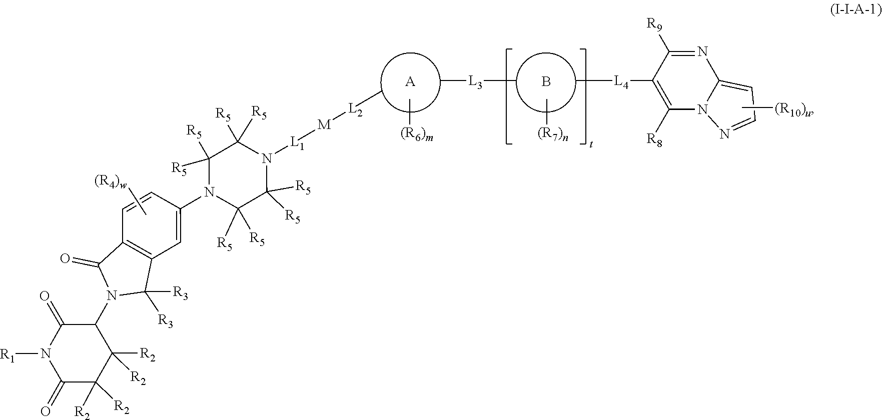

or a pharmaceutically acceptable salt, stereoisomer, solvate, prodrug, isotopic derivative, or tautomer thereof wherein all variables are as defined herein.

In some embodiments, the compound is of Formula (I-I-A-1-a):

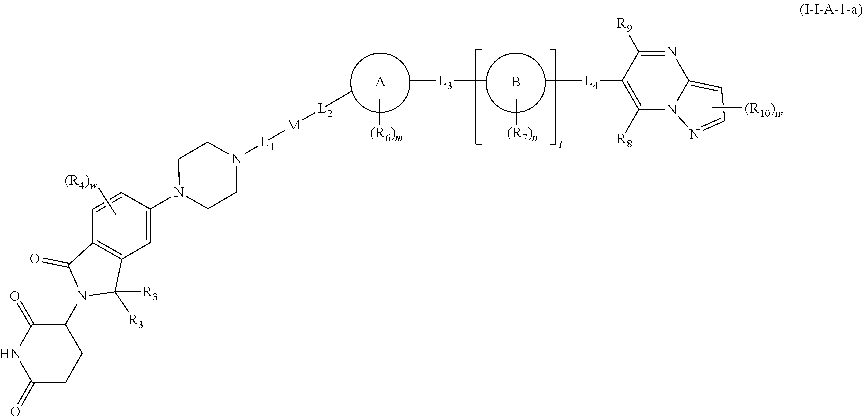

or a pharmaceutically acceptable salt, stereoisomer, solvate, prodrug, isotopic derivative, or tautomer thereof wherein all variables are as defined herein.

In some embodiments, the compound is of Formula (I-I-A-1-a-1):

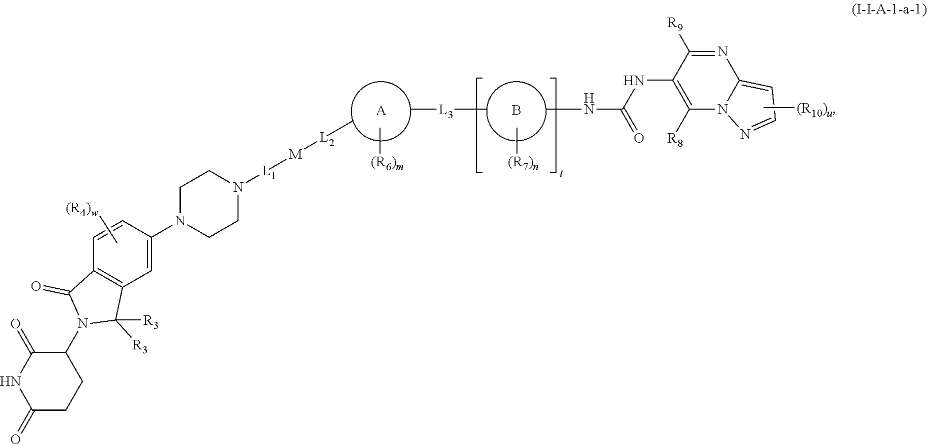

or a pharmaceutically acceptable salt, stereoisomer, solvate, prodrug, isotopic derivative, or tautomer thereof wherein all variables are as defined herein.

In some embodiments, the compound is of Formula (I-I-A-1-a-1-I):

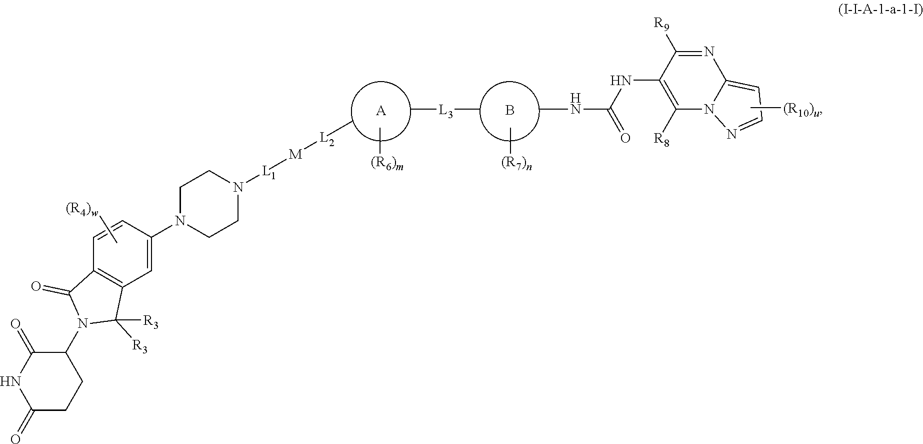

or a pharmaceutically acceptable salt, stereoisomer, solvate, prodrug, isotopic derivative, or tautomer thereof wherein all variables are as defined herein.

In some embodiments, the compound is of Formula (I-I-A-1-a-1-I-A):

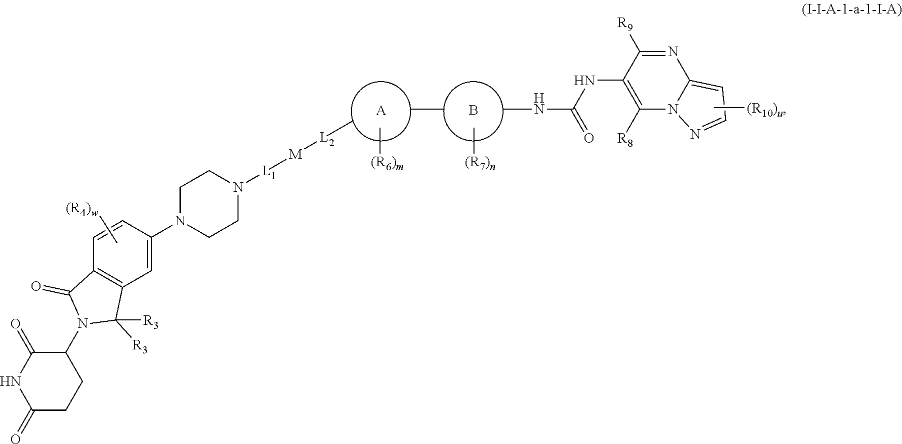

or a pharmaceutically acceptable salt, stereoisomer, solvate, prodrug, isotopic derivative, or tautomer thereof wherein all variables are as defined herein.

In some embodiments, the compound is of Formula (I-I-A-1-a-1-I-A-1):

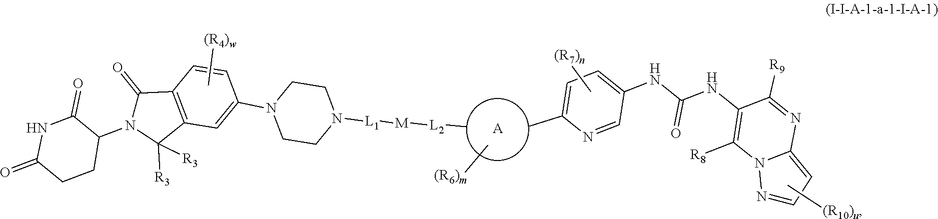

or a pharmaceutically acceptable salt, stereoisomer, solvate, prodrug, isotopic derivative, or tautomer thereof wherein n is selected from 0 and 1; wherein each R₃ is H or two R₃ form ═O; and all other variables are as defined herein.

In some embodiments, the compound is of Formula (I-I-A-1-a-1-I-A-1-I):

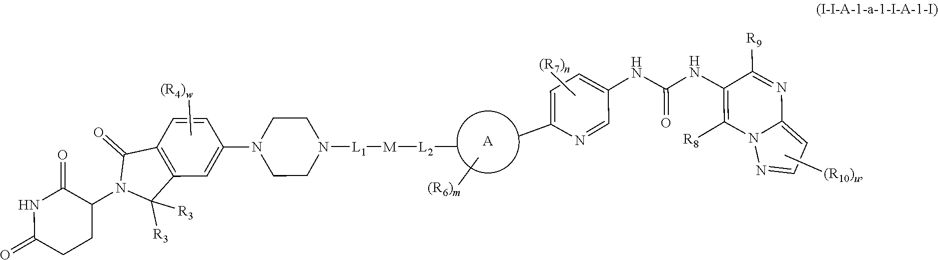

or a pharmaceutically acceptable salt, stereoisomer, solvate, prodrug, isotopic derivative, or tautomer thereof wherein n is selected from 0 and 1; wherein each R₃ is H or two R₃ form ═O; and all other variables are as defined herein.

In some embodiments, the compound is of Formula (I-1):

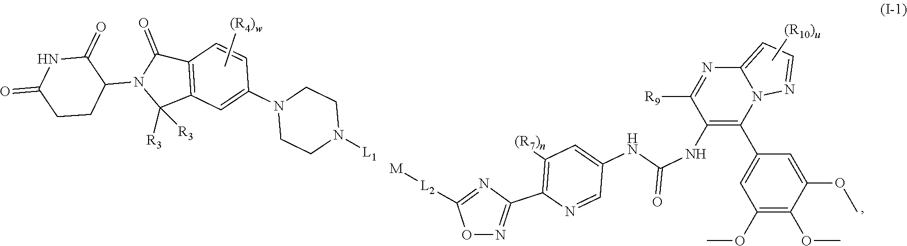

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-2):

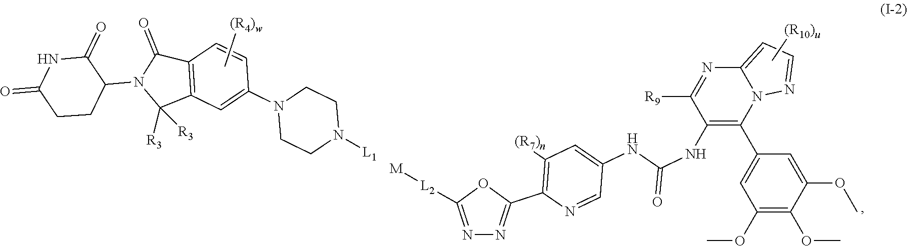

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-3):

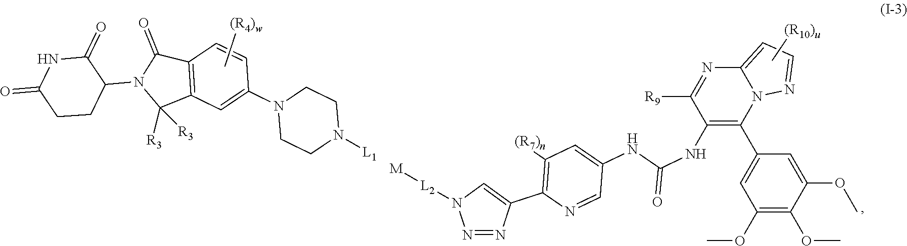

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-4):

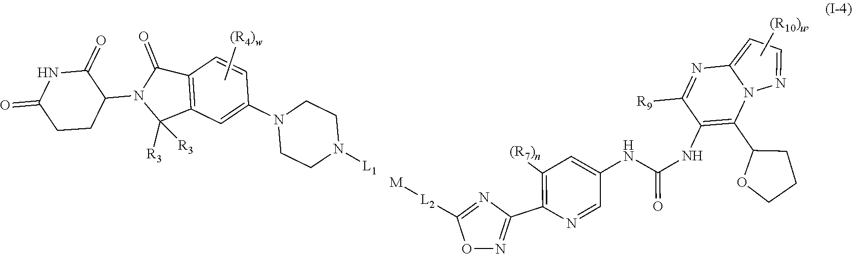

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-5):

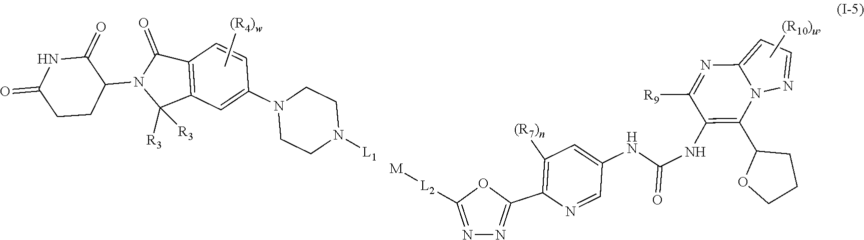

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-6):

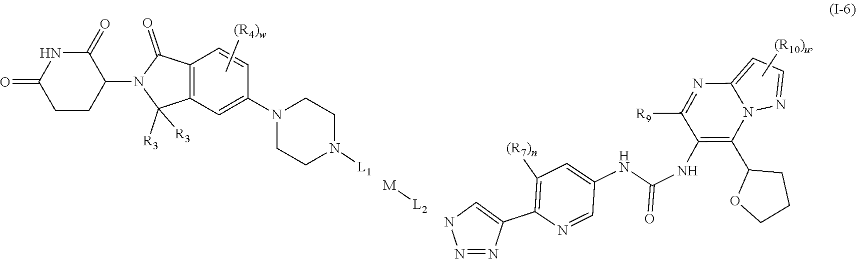

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-7):

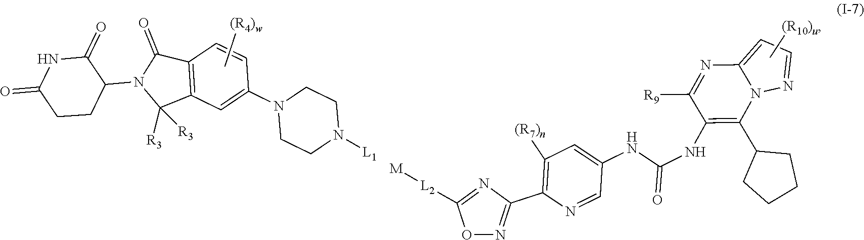

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-8):

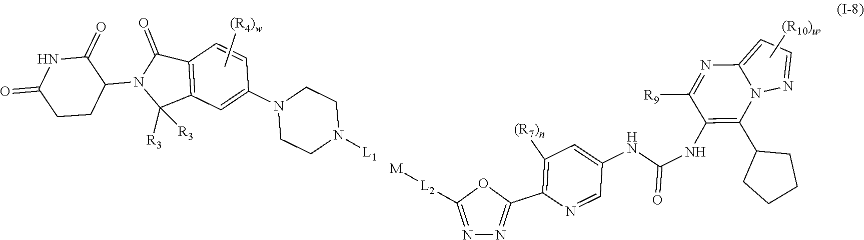

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-9):

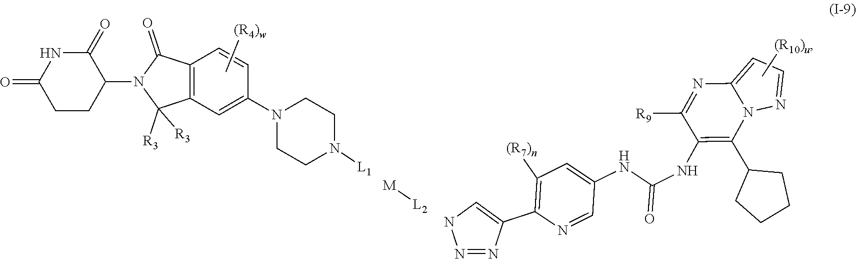

or a pharmaceutically acceptable salt, stereoisomer, solvate, or tautomer thereof, wherein n is an integer selected from 0 and 1; wherein both R₃ are H or two R₃ form ═0, and wherein the remaining variables are as defined herein.

In some embodiments, the compound is of Formula (I-10):

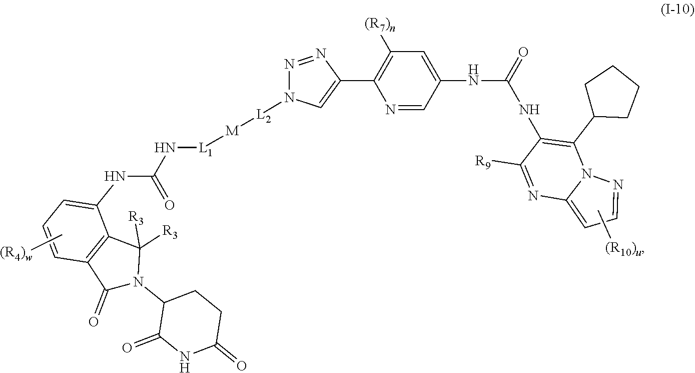

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-11):

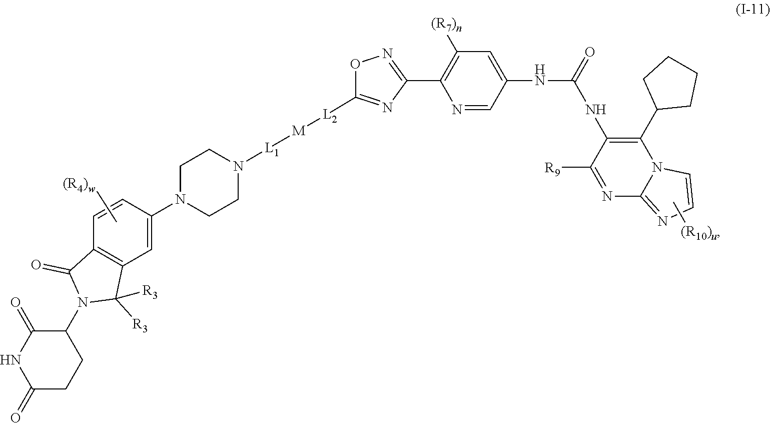

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-12):

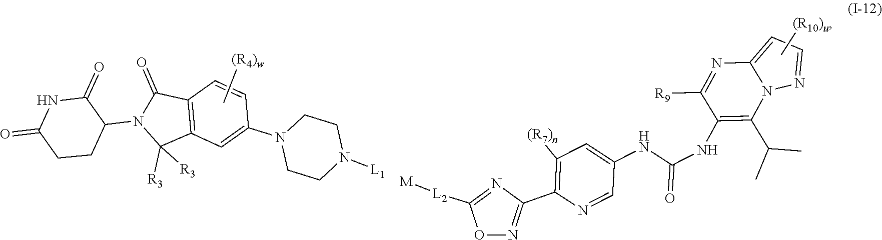

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-13):

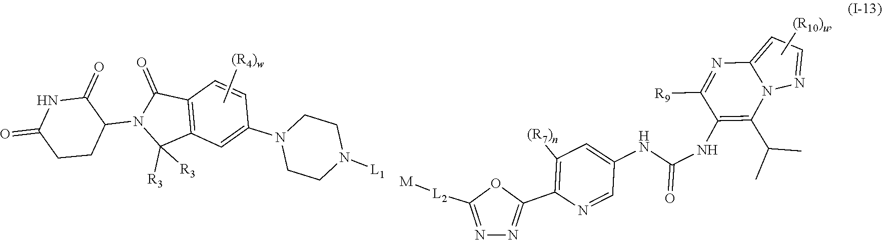

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-14):

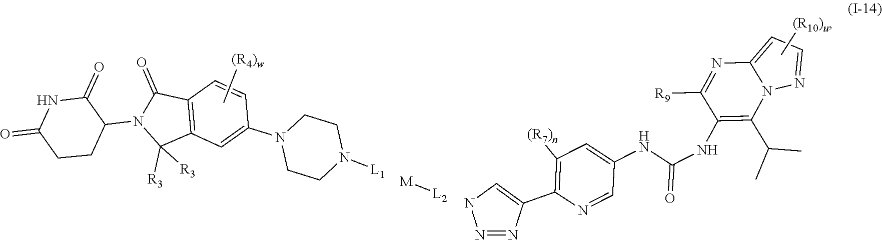

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-15):

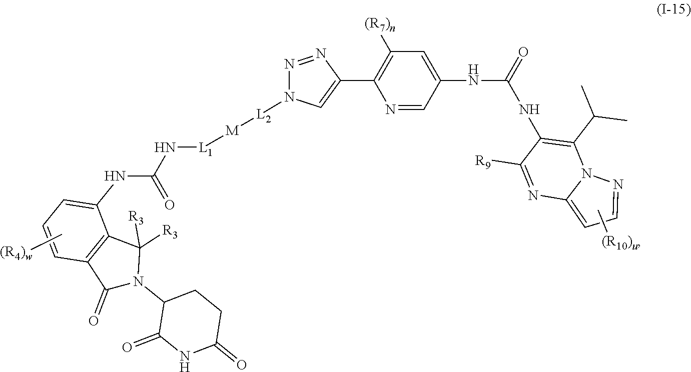

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-16):

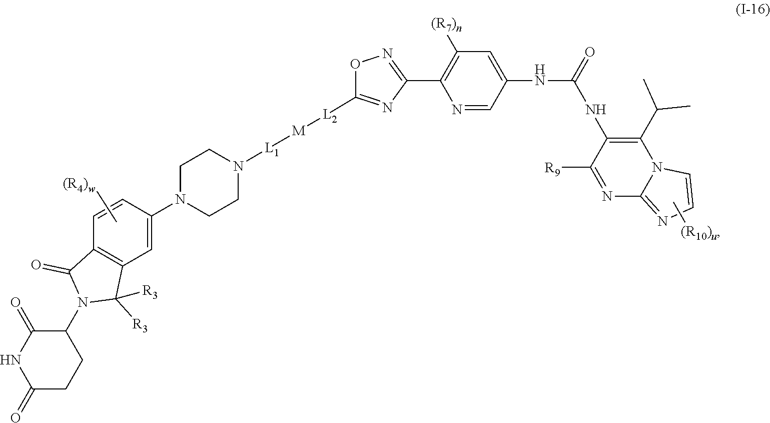

or a pharmaceutically acceptable salt, stereoisomer, solvate, or tautomer thereof, wherein n is an integer selected from 0 and 1; wherein both R₃ are H or two R₃ form ═O, and wherein the remaining variables are as defined herein.

In some embodiments, the compound is of Formula (I-I-A-1-a-1-I-A-1-I-A):

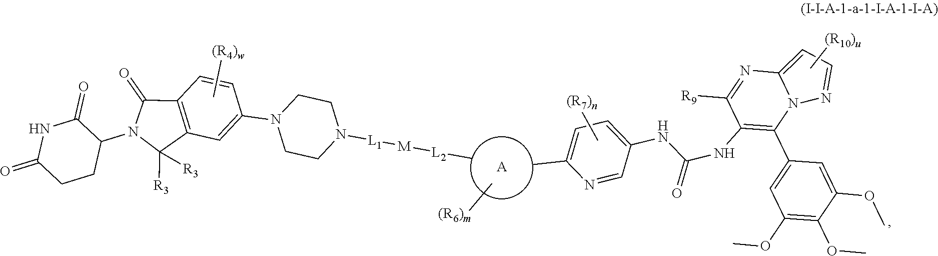

or a pharmaceutically acceptable salt, stereoisomer, solvate, prodrug, isotopic derivative, or tautomer thereof wherein n is selected from 0 and 1; wherein each R₃ is H or two R₃ form ═O; and all other variables are as defined herein.

In some embodiments, the compound is of Formula (I-I-A-1-a-1-I-A-1-I-A-1):

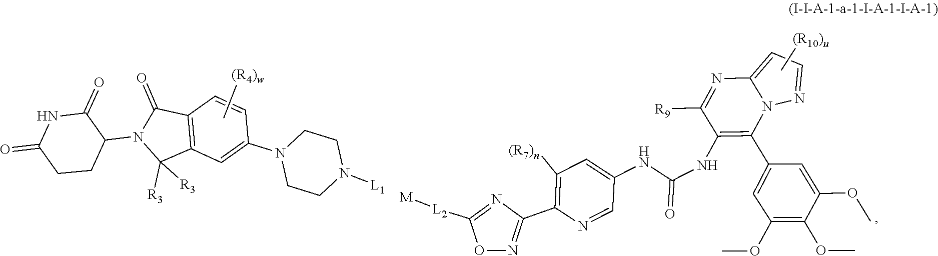

or a pharmaceutically acceptable salt, stereoisomer, solvate, prodrug, isotopic derivative, or tautomer thereof wherein each R₃ is H or two R₃ form ═O; n is selected from 0 and 1; and all other variables are as defined herein.

In some embodiments, the compound is of Formula (I-I-A-1-a-1-I-A-1-I-A-1-a):

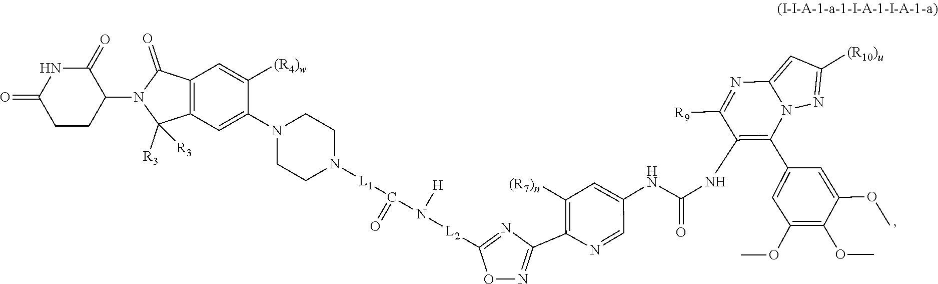

or a pharmaceutically acceptable salt, stereoisomer, solvate, prodrug, isotopic derivative, or tautomer thereof, wherein each R₃ is H or two R₃ form ═O; each from w, u and n is independently selected from 0 and 1; and all other variables are as defined herein.

In some embodiments, the compound is of Formula (I-I-A-1-a-1-I-A-1-I-A-1-a-H):

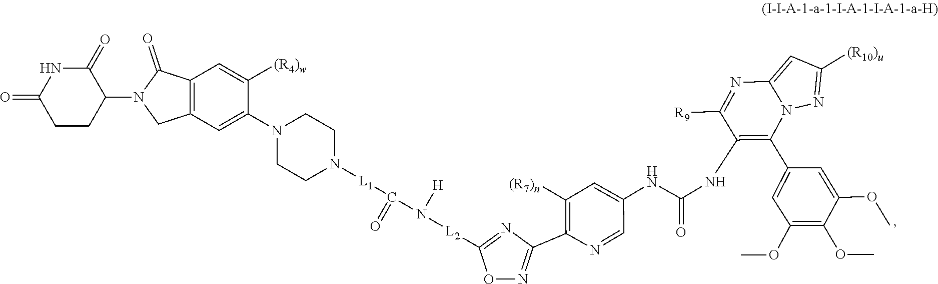

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-A-1-a-O):

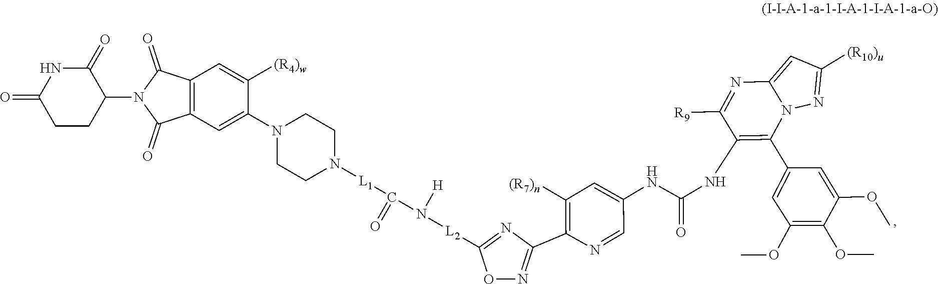

or a pharmaceutically acceptable salt, stereoisomer, solvate, prodrug, isotopic derivative, or tautomer thereof, wherein w, u and n are each independently selected from 0 and 1; and all other variables are as defined herein.

In some embodiments, the compound is of Formula (I-I-A-1-a-1-I-A-1-I-A-2):

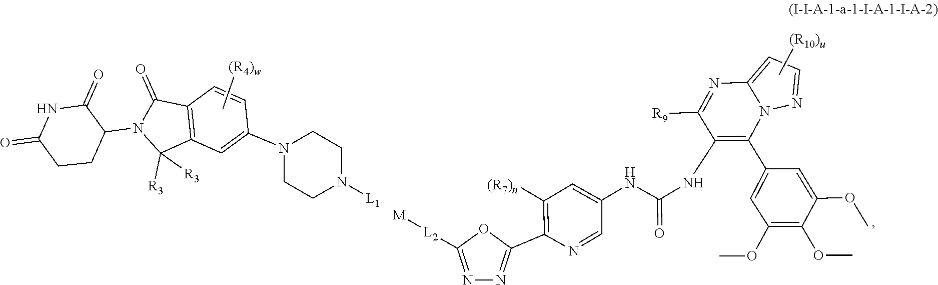

or a pharmaceutically acceptable salt, stereoisomer, solvate, prodrug, isotopic derivative, or tautomer thereof wherein each R₃ is H or two R₃ form ═O; n is selected from 0 and 1; and all other variables are as defined herein.

In some embodiments, the compound is of Formula (I-I-A-1-a-1-I-A-1-I-A-2-H):

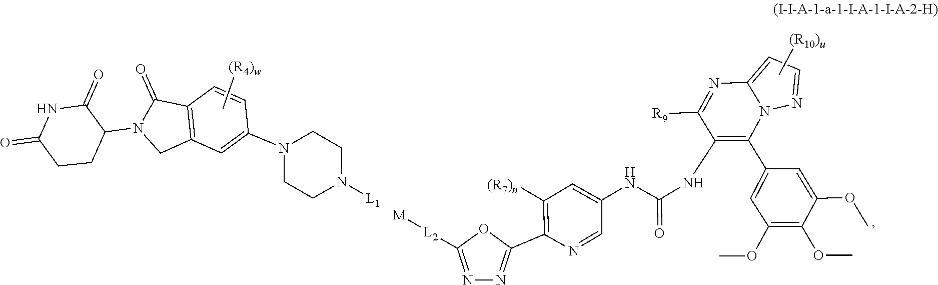

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-A-2-O):

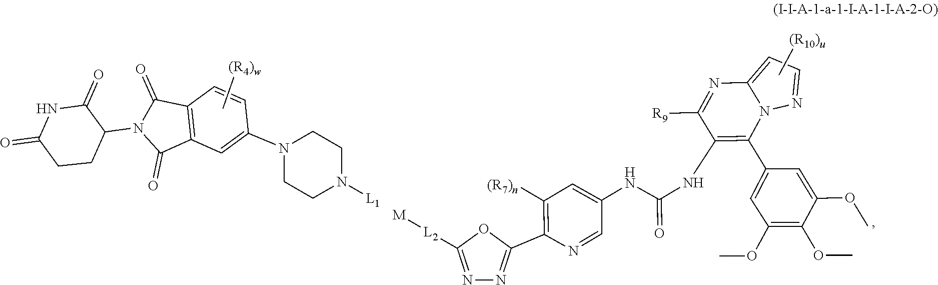

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-A-3):

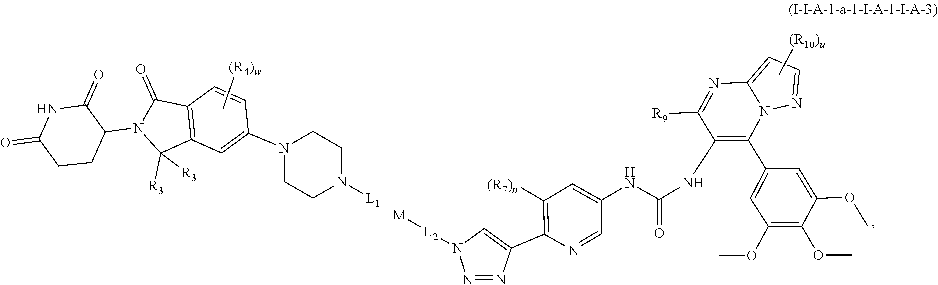

or a pharmaceutically acceptable salt, stereoisomer, solvate, prodrug, isotopic derivative, or tautomer thereof wherein each R₃ is H or two R₃ form ═O; n is selected from 0 and 1; and all other variables are as defined herein.

In some embodiments, the compound is of Formula (I-I-A-1-a-1-I-A-1-I-A-3-H):

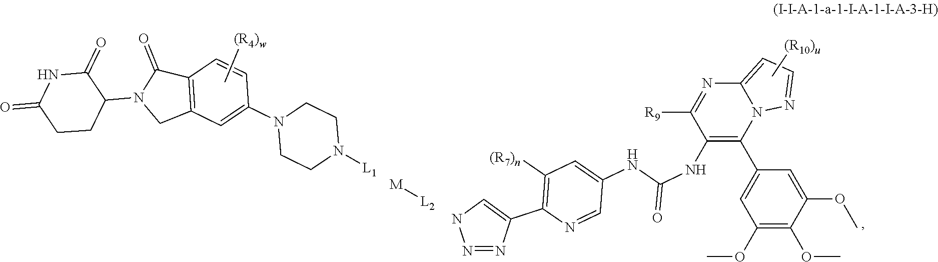

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-A-3-O):

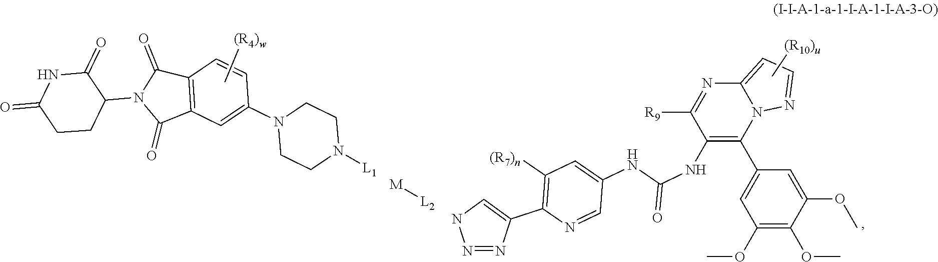

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-B):

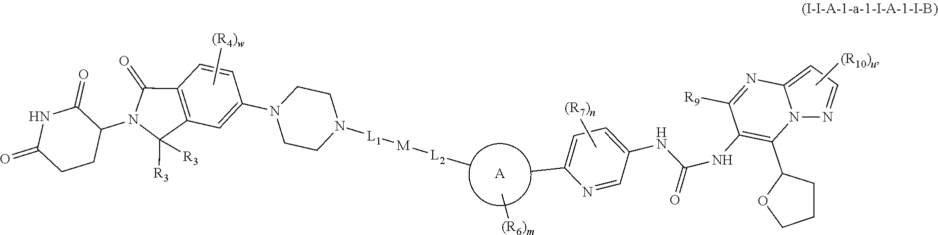

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-B-1):

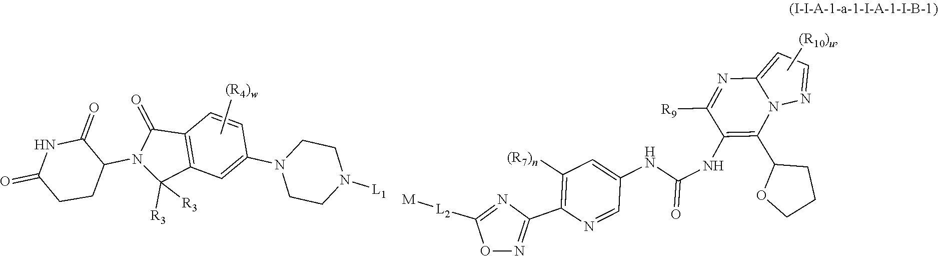

or a pharmaceutically acceptable salt, stereoisomer, solvate, prodrug, isotopic derivative, or tautomer thereof wherein each R₃ is H or two R₃ form ═O; n is selected from 0 and 1; and all other variables are as defined herein.

In some embodiments, the compound is of Formula (I-I-A-1-a-1-I-A-1-I-B-1-a):

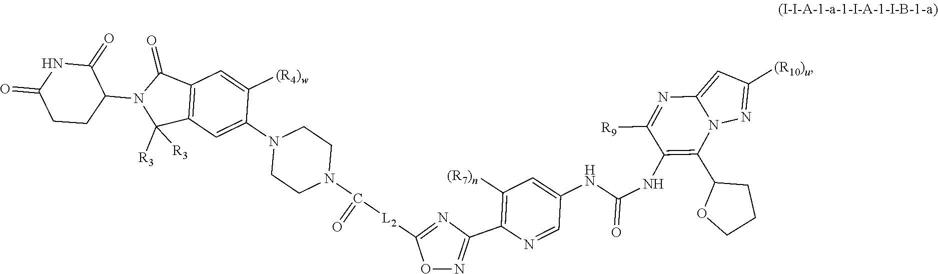

or a pharmaceutically acceptable salt, stereoisomer, solvate, prodrug, isotopic derivative, or tautomer thereof, wherein each from w, u and n is independently selected from 0 and 1; each R₃ is H or two R₃ are ═O; and all other variables are as defined herein.

In some embodiments, the compound is of Formula (I-I-A-1-a-1-I-A-1-I-B-1-a-H):

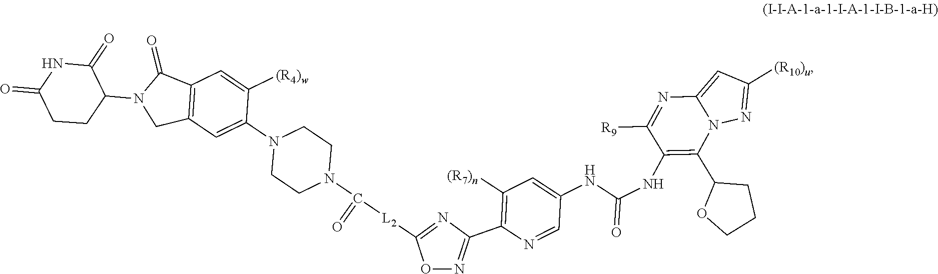

or a pharmaceutically acceptable salt, stereoisomer, solvate, prodrug, isotopic derivative, or tautomer thereof, wherein each from w, u, and n is independently selected from 0 and 1; and all other variables are as defined herein.

In some embodiments, the compound is of Formula (I-I-A-1-a-1-I-A-1-I-B-1-a-O):

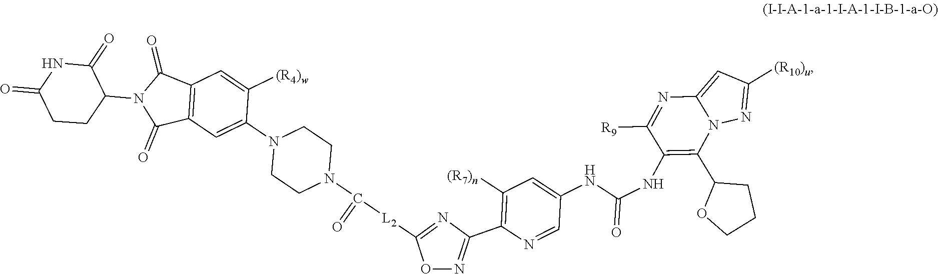

or a pharmaceutically acceptable salt, stereoisomer, solvate, prodrug, isotopic derivative, or tautomer thereof, wherein each from w, u, and n is independently selected from 0 and 1; and all other variables are as defined herein.

In some embodiments, the compound is of Formula (I-I-A-1-a-1-I-A-1-I-B-2):

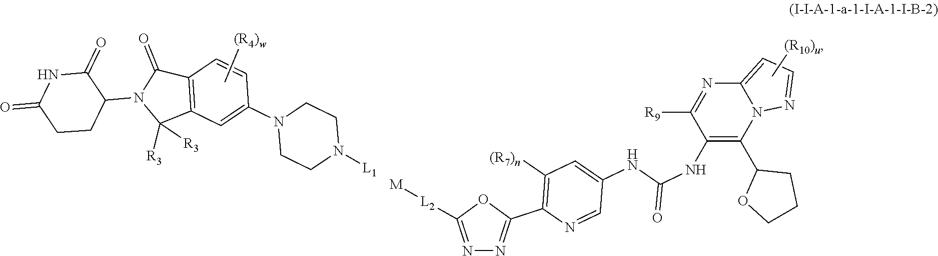

or a pharmaceutically acceptable salt, stereoisomer, solvate, prodrug, isotopic derivative, or tautomer thereof wherein each R₃ is H or two R₃ are ═O; n is selected from 0 and 1; and all other variables are as defined herein.

In some embodiments, the compound is of Formula (I-I-A-1-a-1-I-A-1-I-B-2-H):

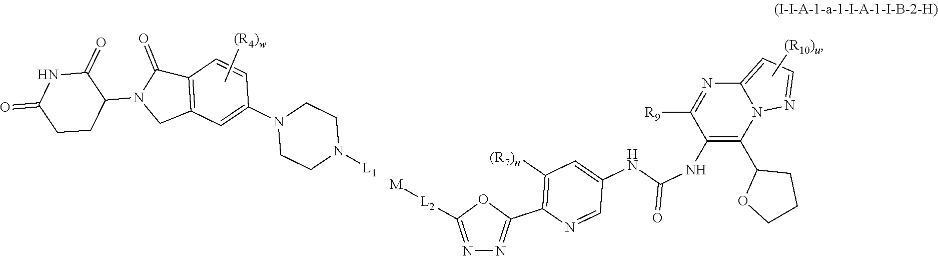

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-B-2-O):

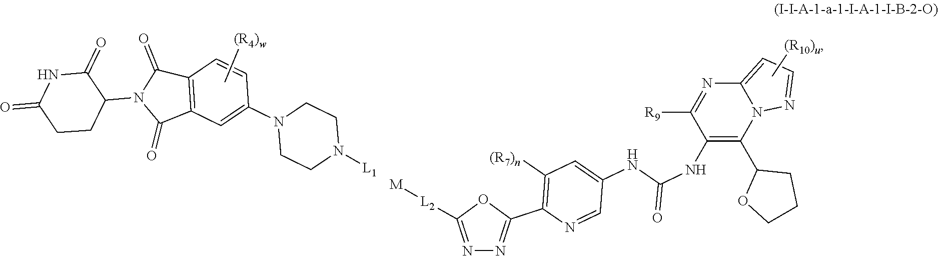

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-B-3):

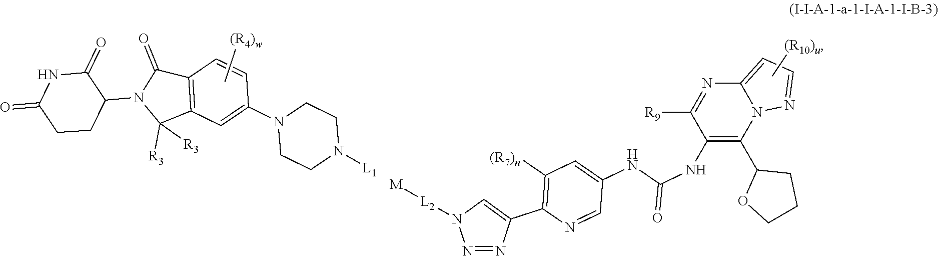

or a pharmaceutically acceptable salt, stereoisomer, solvate, prodrug, isotopic derivative, or tautomer thereof wherein each R₃ is H or two R₃ are ═O; n is selected from 0 and 1; and all other variables are as defined herein.

In some embodiments, the compound is of Formula (I-I-A-1-a-1-I-A-1-I-B-3-H):

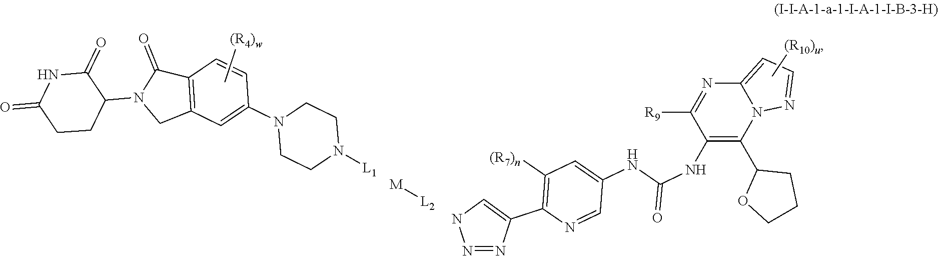

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-B-3-O):

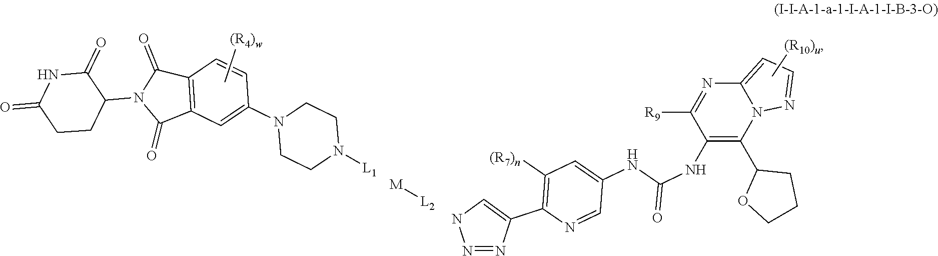

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-C):

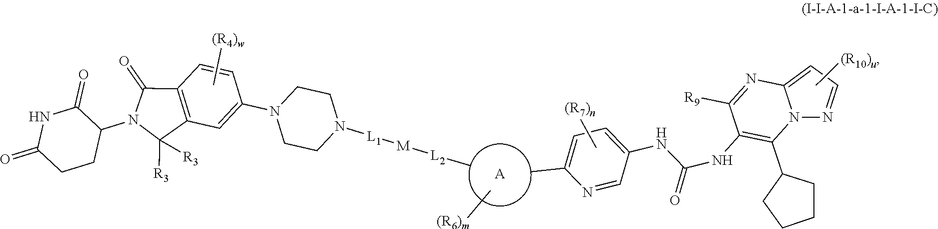

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-C-1):

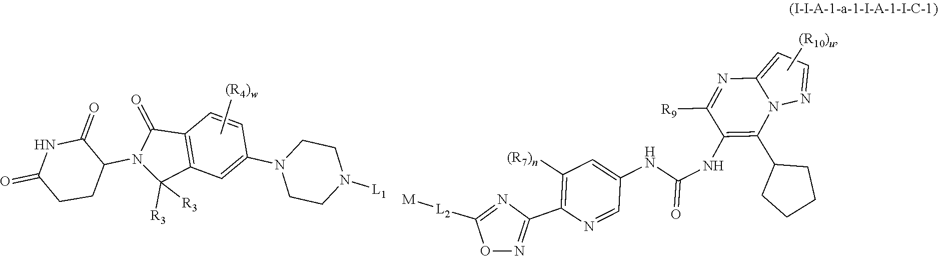

or a pharmaceutically acceptable salt, stereoisomer, solvate, prodrug, isotopic derivative, or tautomer thereof wherein each R₃ is H or two R₃ are ═O; each n is independently selected from 0 and 1; and all other variables are as defined herein.

In some embodiments, the compound is of Formula (I-I-A-1-a-1-I-A-1-I-C-1-a):

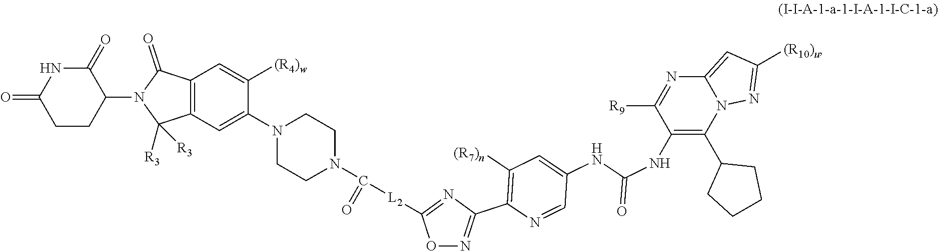

or a pharmaceutically acceptable salt, stereoisomer, solvate, prodrug, isotopic derivative, or tautomer thereof, wherein each from w, u and n is independently selected from 0 and 1; each R₃ is H or two R₃ are ═O; and all other variables are as defined herein.

In some embodiments, the compound is of Formula (I-I-A-1-a-1-I-A-1-I-C-1-a-H):

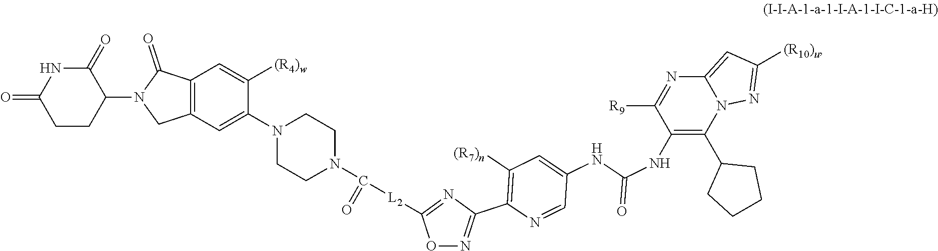

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-C-1-a-O):

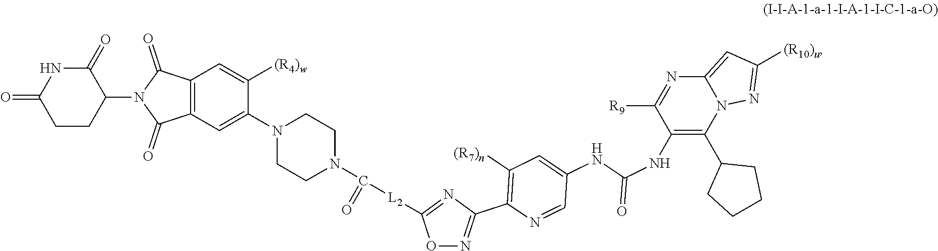

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-C-1-b):

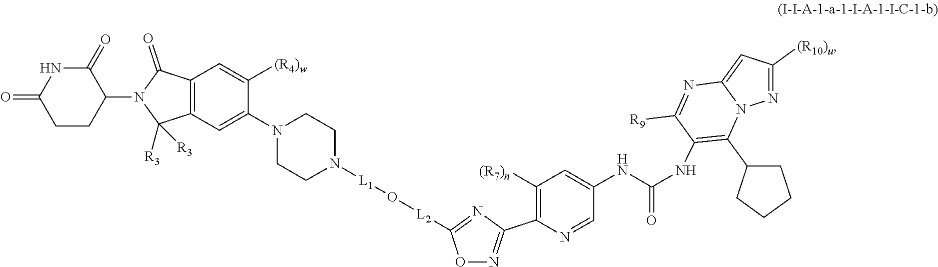

or a pharmaceutically acceptable salt, stereoisomer, solvate, prodrug, isotopic derivative, or tautomer thereof, wherein each from w, u and n is independently selected from 0 and 1; each R₃ is H or two R₃ are ═O; and all other variables are as defined herein.

In some embodiments, the compound is of Formula (I-I-A-1-a-1-I-A-1-I-C-1-b-H):

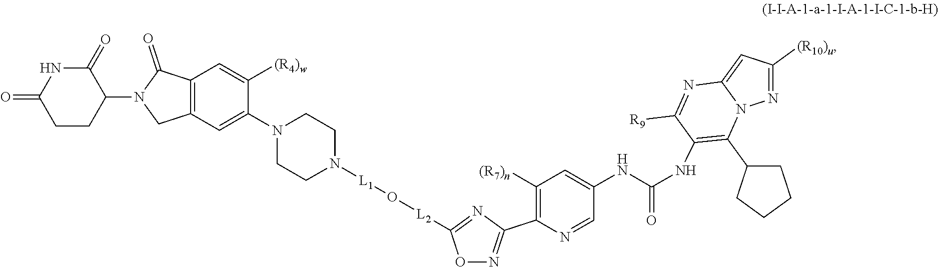

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-C-1-b-O):

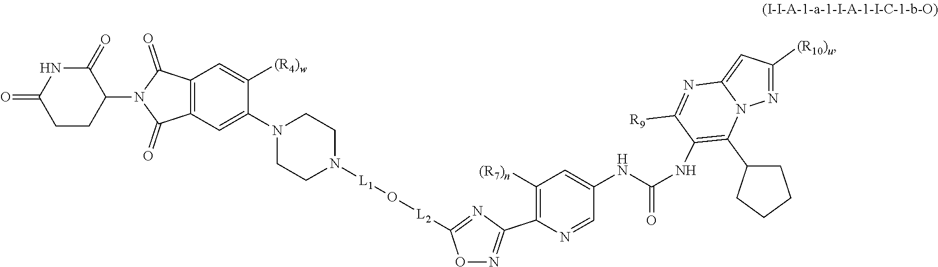

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-C-1-c):

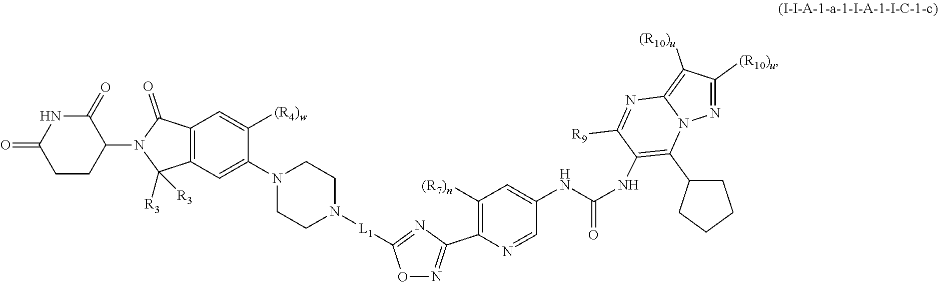

or a pharmaceutically acceptable salt, stereoisomer, solvate, prodrug, isotopic derivative, or tautomer thereof, wherein each from w, u and n is independently selected from 0 and 1; each R₃ is H or two R₃ are ═O; and all other variables are as defined herein.

In some embodiments, the compound is of Formula (I-I-A-1-a-1-I-A-1-I-C-1-c-H):

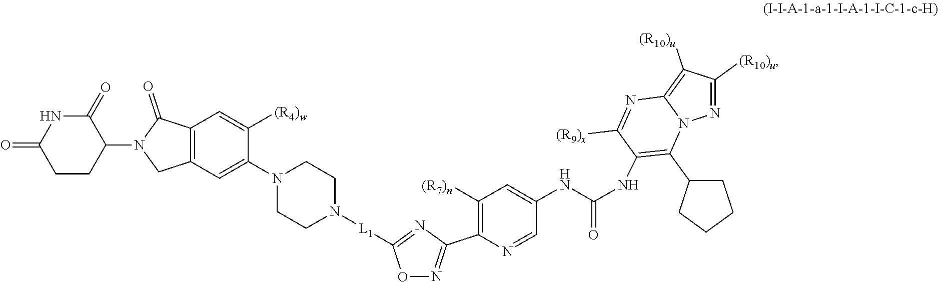

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-C-1-c-O).

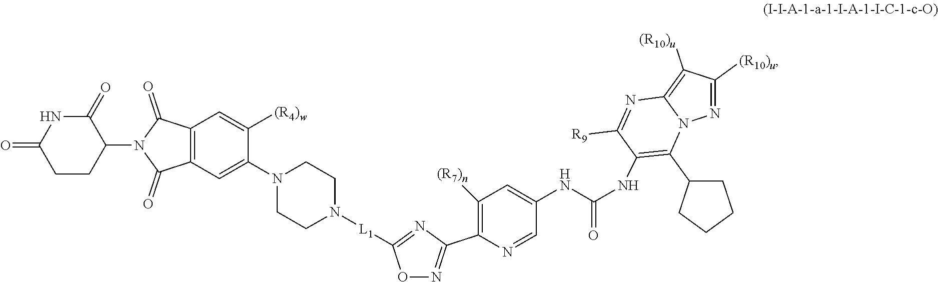

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-C-2):

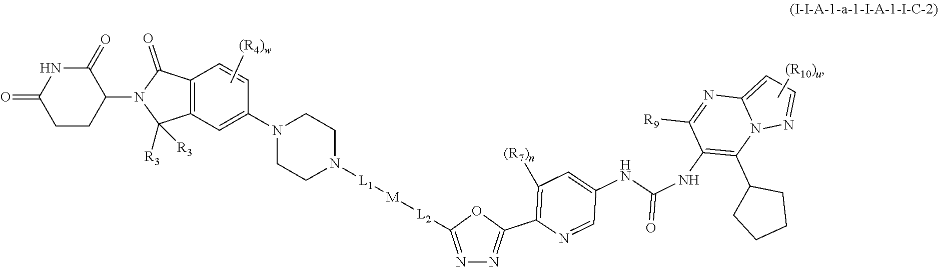

or a pharmaceutically acceptable salt, stereoisomer, solvate, prodrug, isotopic derivative, or tautomer thereof wherein n is selected from 0 and 1; each R₃ is H or two R₃ are ═O; and all other variables are as defined herein.

In some embodiments, the compound is of Formula (I-I-A-1-a-1-I-A-1-I-C-2-a):

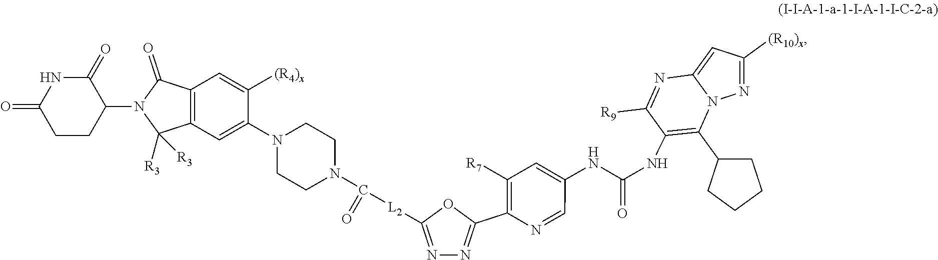

or a pharmaceutically acceptable salt, stereoisomer, solvate, prodrug, isotopic derivative, or tautomer thereof, wherein each x independently selected from 0 and 1; each R₃ is H or two R₃ are ═O; all other variables are as defined herein.

In some embodiments, the compound is of Formula (I-I-A-1-a-1-I-A-1-I-C-2-a-H):

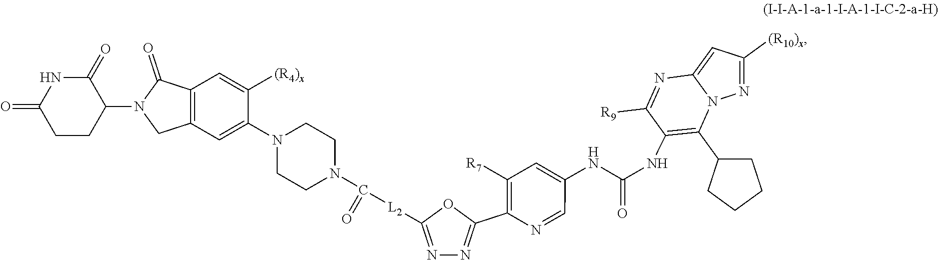

or a pharmaceutically acceptable salt, stereoisomer, solvate, prodrug, isotopic derivative, or tautomer thereof, wherein each x independently selected from 0 and 1; and all other variables are as defined herein.

In some embodiments, the compound is of Formula (I-I-A-1-a-1-I-A-1-I-C-2-a-O):

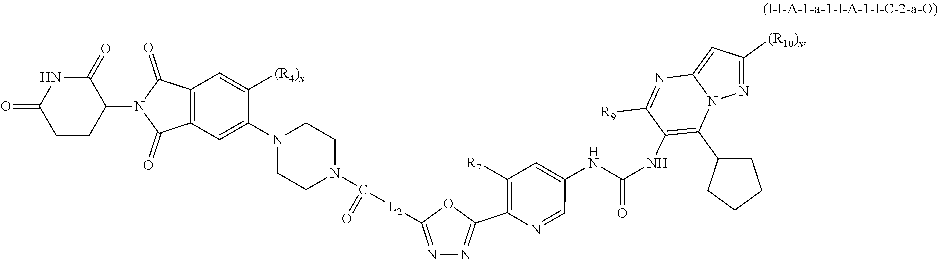

or a pharmaceutically acceptable salt, stereoisomer, solvate, prodrug, isotopic derivative, or tautomer thereof, wherein each x independently selected from 0 and 1; and all other variables are as defined herein.

In some embodiments, the compound is of Formula (I-I-A-1-a-1-I-A-1-I-C-3):

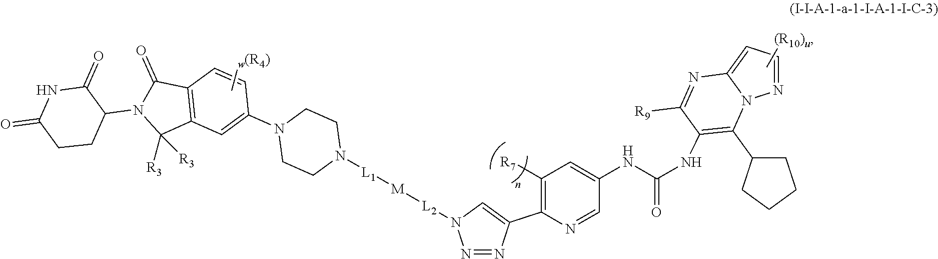

or a pharmaceutically acceptable salt, stereoisomer, solvate, prodrug, isotopic derivative, or tautomer thereof wherein n is selected from 0 and 1; each R₃ is H or two R₃ are ═O; and all other variables are as defined herein.

In some embodiments, the compound is of Formula (I-I-A-1-a-1-I-A-1-I-C-3-a):

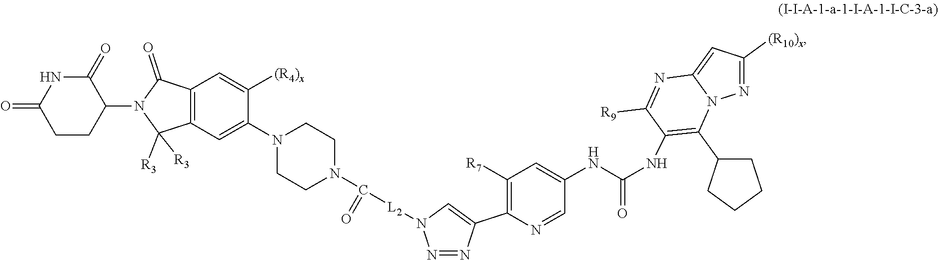

or a pharmaceutically acceptable salt, stereoisomer, solvate, prodrug, isotopic derivative, or tautomer thereof, wherein each x independently selected from 0 and 1; each R₃ is H or two R₃ are ═O; and all other variables are as defined herein.

In some embodiments, the compound is of Formula (I-I-A-1-a-1-I-A-1-I-C-3-a-H):

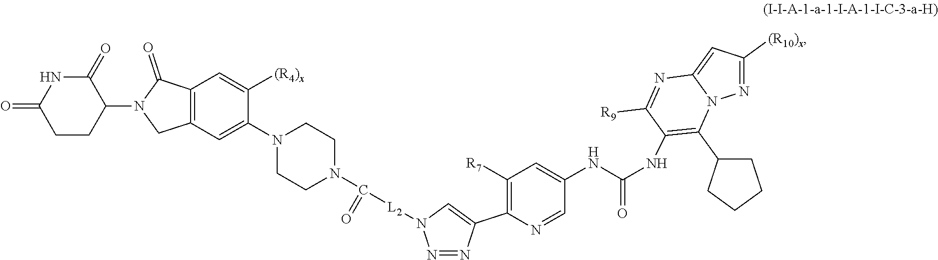

or a pharmaceutically acceptable salt, stereoisomer, solvate, prodrug, isotopic derivative, or tautomer thereof, wherein each x independently selected from 0 and 1; and all other variables are as defined herein.

In some embodiments, the compound is of Formula (I-I-A-1-a-1-I-A-1-I-C-3-a-O):

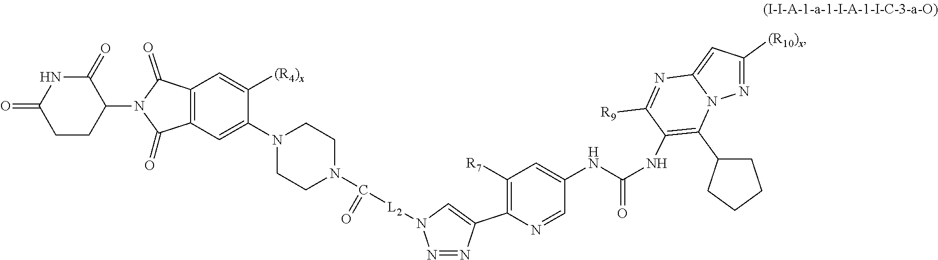

or a pharmaceutically acceptable salt, stereoisomer, solvate, prodrug, isotopic derivative, or tautomer thereof, wherein each x independently selected from 0 and 1; and all other variables are as defined herein.

In some embodiments, the compound is of Formula (I-I-A-1-a-1-I-A-1-I-D):

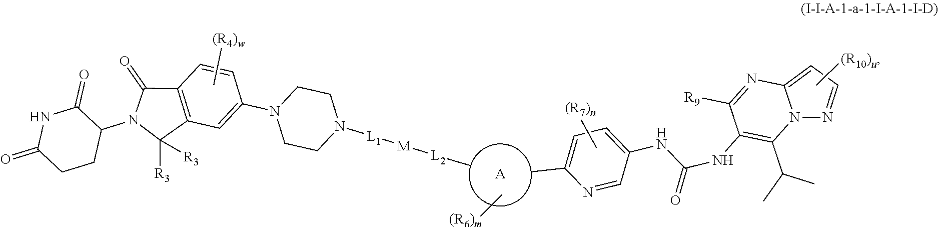

or a pharmaceutically acceptable salt, stereoisomer, solvate, prodrug, isotopic derivative, or tautomer thereof wherein n is selected from 0 and 1; and all other variables are as defined herein.

In some embodiments, the compound is of Formula (I-I-A-1-a-1-I-A-1-I-D-1):

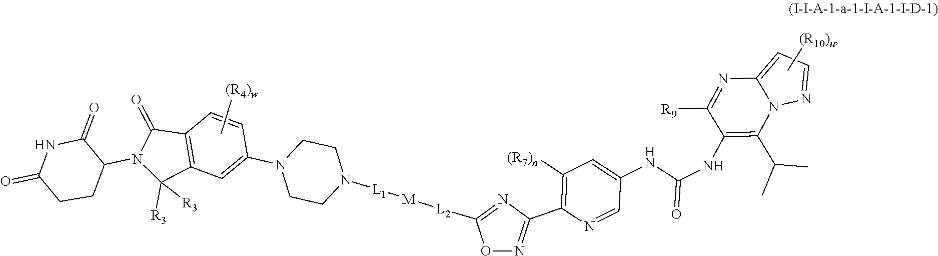

or a pharmaceutically acceptable salt, stereoisomer, solvate, prodrug, isotopic derivative, or tautomer thereof wherein each R₃ is H or two R₃ are ═O; each n is independently selected from 0 and 1; and all other variables are as defined herein.

In some embodiments, the compound is of Formula (I-I-A-1-a-1-I-A-1-I-D-1-a):

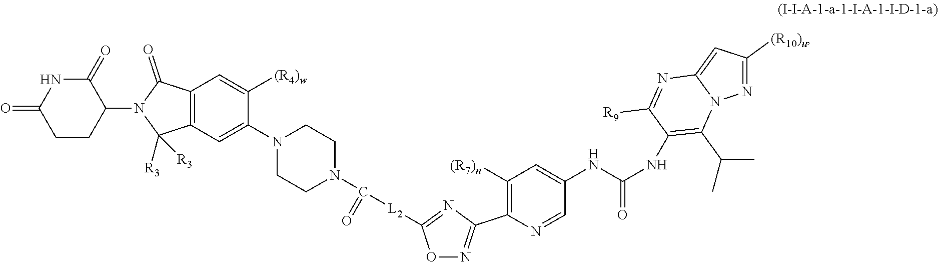

or a pharmaceutically acceptable salt, stereoisomer, solvate, prodrug, isotopic derivative, or tautomer thereof, wherein each from w, u and n is independently selected from 0 and 1; each R₃ is H or two R₃ are ═O; and all other variables are as defined herein.

In some embodiments, the compound is of Formula (I-I-A-1-a-1-I-A-1-I-D-1-a-H):

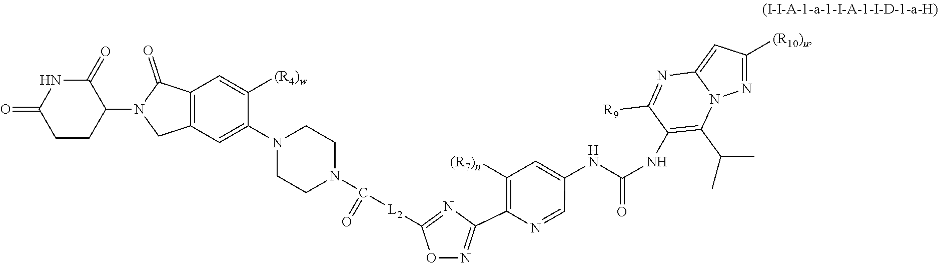

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-D-1-a-O):

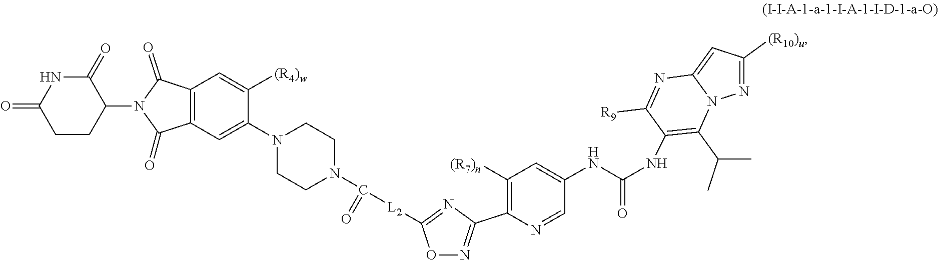

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-D-1-b):

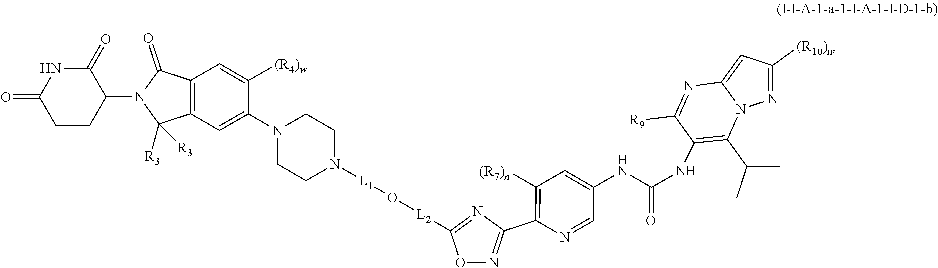

or a pharmaceutically acceptable salt, stereoisomer, solvate, prodrug, isotopic derivative, or tautomer thereof, wherein each from w, u and n is independently selected from 0 and 1; each R₃ is H or two R₃ are ═O; and all other variables are as defined herein.

In some embodiments, the compound is of Formula (I-I-A-1-a-1-I-A-1-I-D-1-b-H):

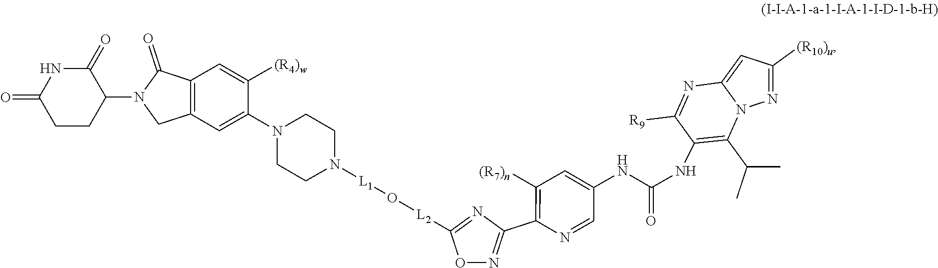

or a pharmaceutically acceptable salt, stereoisomer, solvate, prodrug, isotopic derivative, or tautomer thereof, wherein each from w, u and n is independently selected from 0 and 1; and all other variables are as defined herein.

In some embodiments, the compound is of Formula (I-I-A-1-a-1-I-A-1-I-D-1-b-O):

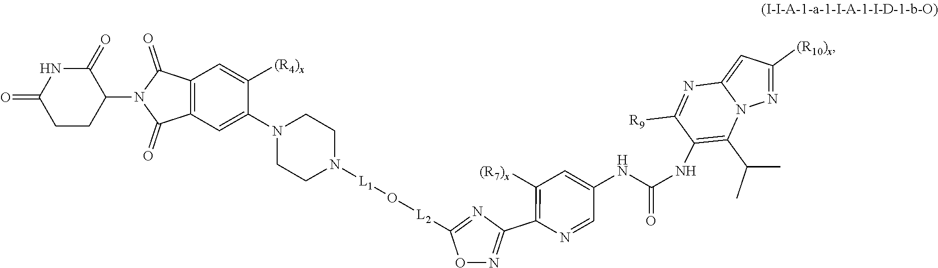

or a pharmaceutically acceptable salt, stereoisomer, solvate, prodrug, isotopic derivative, or tautomer thereof, wherein each x is independently selected from 0 and 1; and all other variables are as defined herein.

In some embodiments, the compound is of Formula (I-I-A-1-a-1-I-A-1-I-D-1-c):

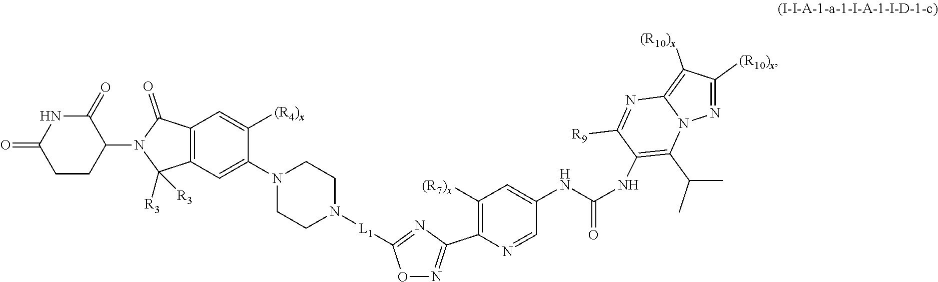

or a pharmaceutically acceptable salt, stereoisomer, solvate, prodrug, isotopic derivative, or tautomer thereof, wherein each x is independently selected from 0 and 1; each R₃ is H or two R₃ are ═O; and all other variables are as defined herein.

In some embodiments, the compound is of Formula (I-I-A-1-a-1-I-A-1-I-D-1-c-H):

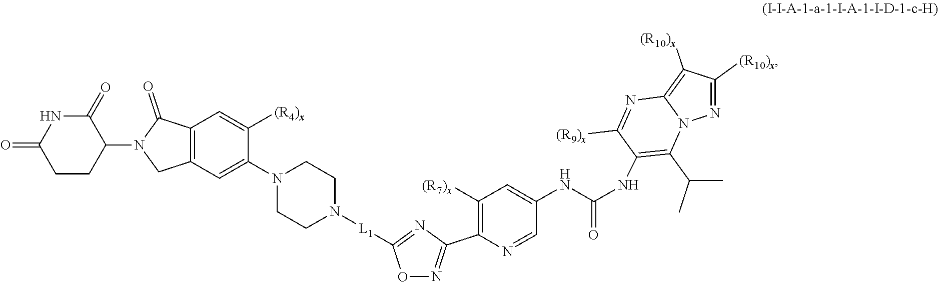

or a pharmaceutically acceptable salt, stereoisomer, solvate, prodrug, isotopic derivative, or tautomer thereof, wherein each x is independently selected from 0 and 1; and all other variables are as defined herein.

In some embodiments, the compound is of Formula (I-I-A-1-a-1-I-A-1-I-D-1-c-O):

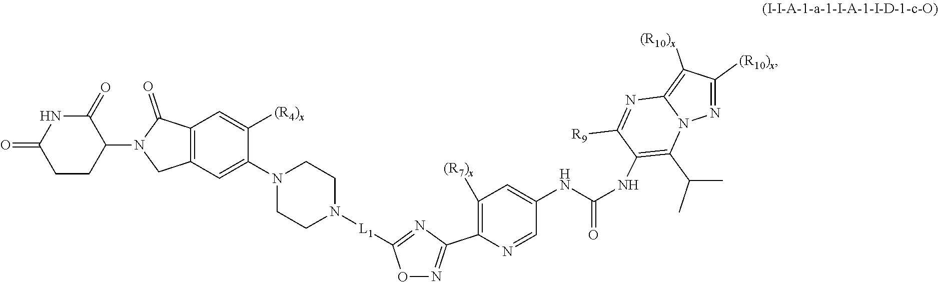

or a pharmaceutically acceptable salt, stereoisomer, solvate, prodrug, isotopic derivative, or tautomer thereof, wherein each x is independently selected from 0 and 1; and all other variables are as defined herein.

In some embodiments, the compound is of Formula (I-I-A-2):

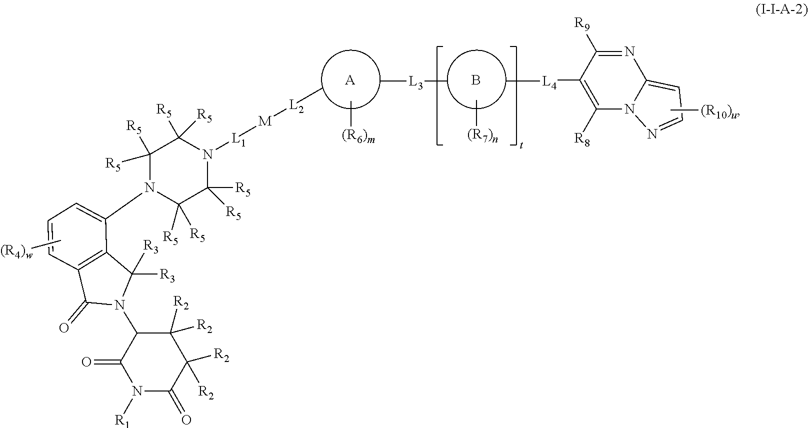

or a pharmaceutically acceptable salt, stereoisomer, solvate, prodrug, isotopic derivative, or tautomer thereof wherein all variables are as defined herein.

In some embodiments, the compound is of Formula (I-I-A-2-a):

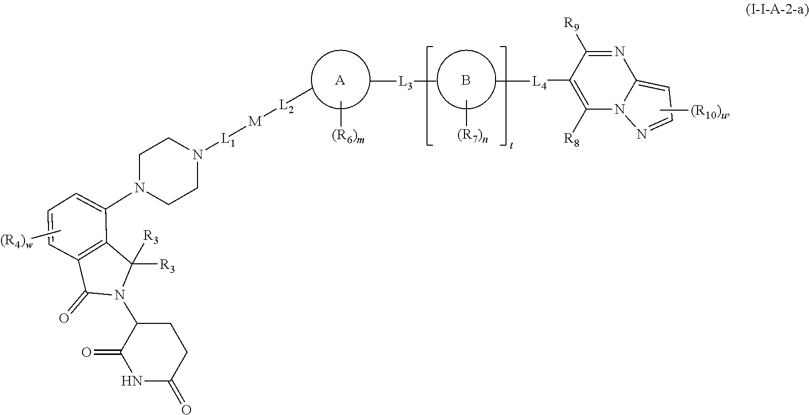

or a pharmaceutically acceptable salt, stereoisomer, solvate, prodrug, isotopic derivative, or tautomer thereof wherein all variables are as defined herein.

In some embodiments, the compound is of Formula (I-I-A-2-a-1):

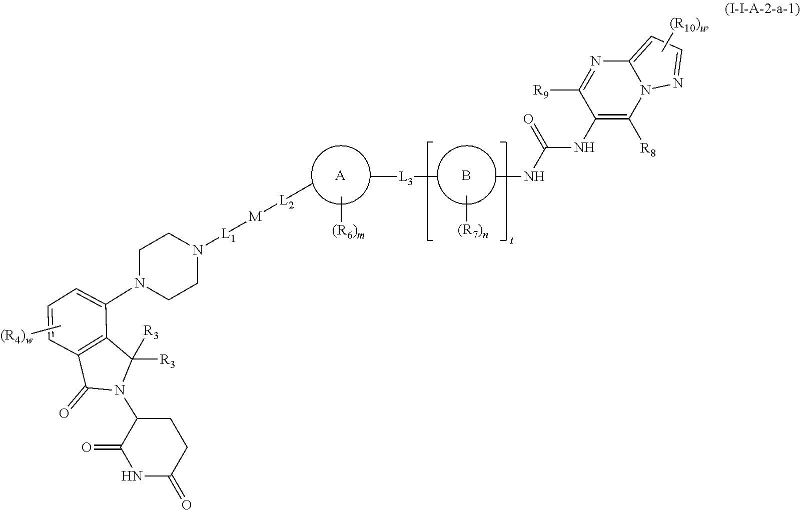

or a pharmaceutically acceptable salt, stereoisomer, solvate, prodrug, isotopic derivative, or tautomer thereof wherein all variables are as defined herein.

In some embodiments, the compound is of Formula (I-I-A-2-a-1-I):

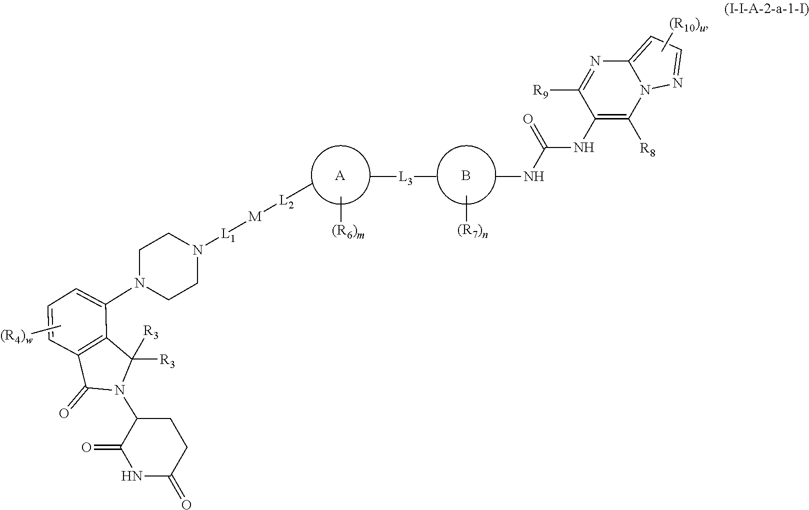

or a pharmaceutically acceptable salt, stereoisomer, solvate, prodrug, isotopic derivative, or tautomer thereof wherein all variables are as defined herein.

In some embodiments, the compound is of Formula (I-I-A-2-a-1-I-A):

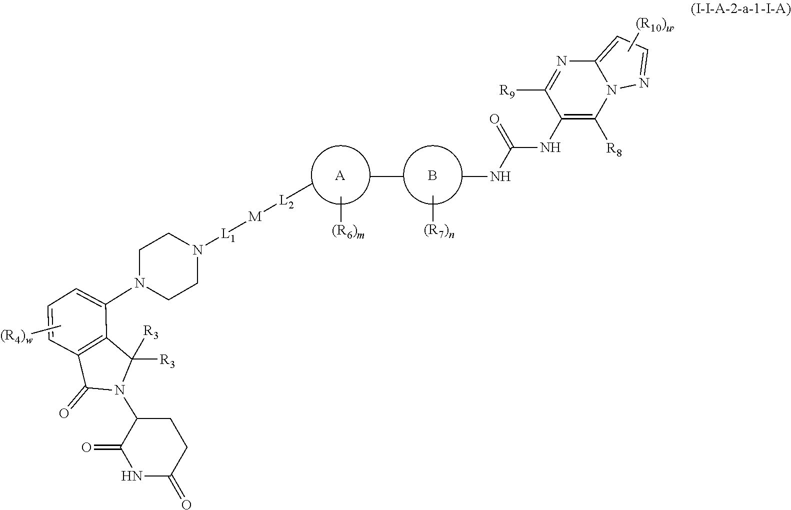

or a pharmaceutically acceptable salt, stereoisomer, solvate, prodrug, isotopic derivative, or tautomer thereof wherein all variables are as defined herein.

In some embodiments, the compound is of Formula (I-I-B):

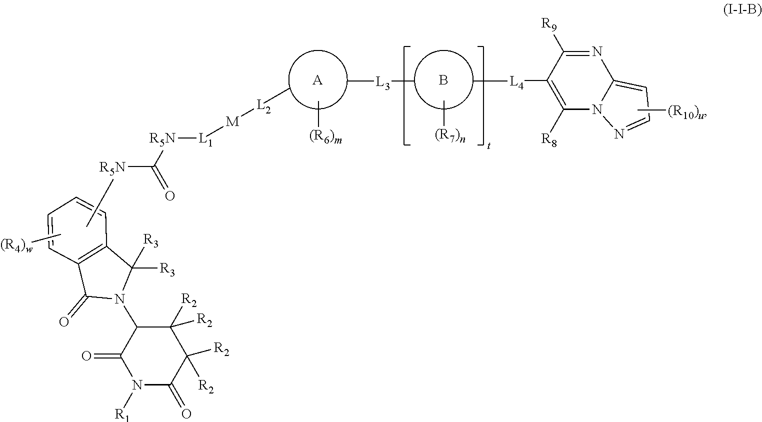

or a pharmaceutically acceptable salt, stereoisomer, solvate, prodrug, isotopic derivative, or tautomer thereof wherein all variables are as defined herein.

In some embodiments, the compound is of Formula (I-I-B-1):

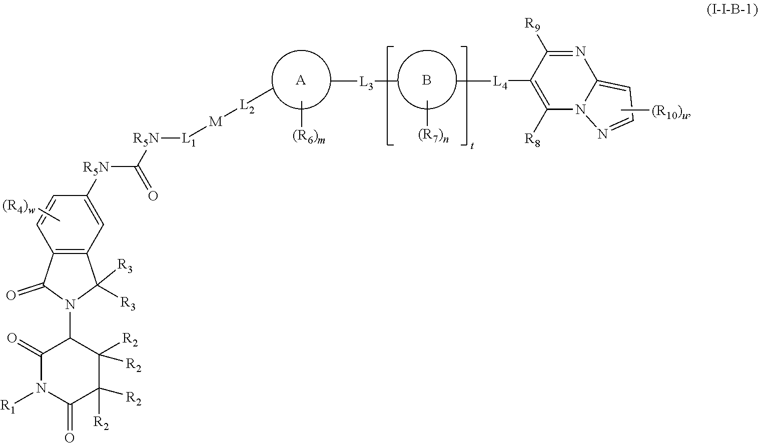

or a pharmaceutically acceptable salt, stereoisomer, solvate, prodrug, isotopic derivative, or tautomer thereof wherein all variables are as defined herein.

In some embodiments, the compound is of Formula (I-I-B-1-a):

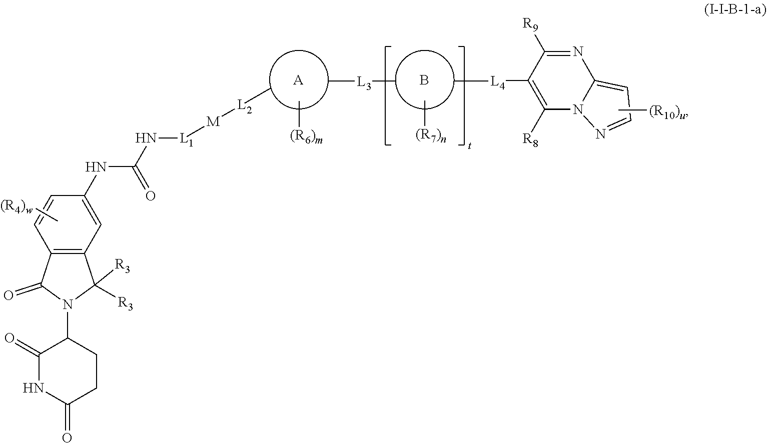

or a pharmaceutically acceptable salt, stereoisomer, solvate, prodrug, isotopic derivative, or tautomer thereof wherein all variables are as defined herein.

In some embodiments, the compound is of Formula (I-I-B-1-a-1):

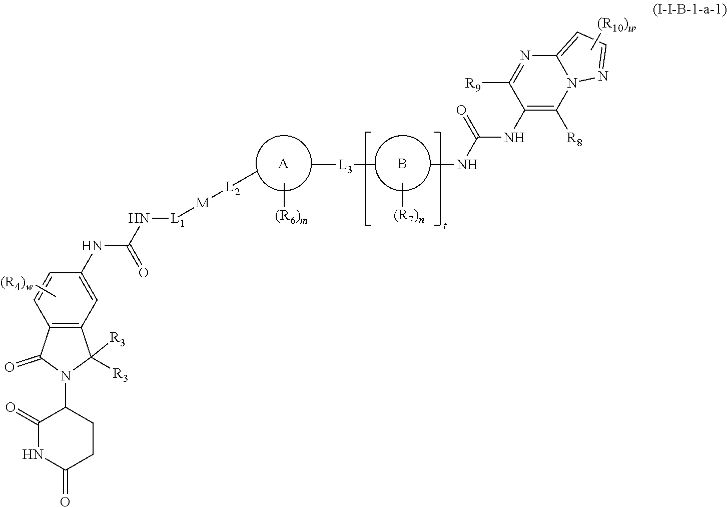

or a pharmaceutically acceptable salt, stereoisomer, solvate, prodrug, isotopic derivative, or tautomer thereof wherein all variables are as defined herein.

In some embodiments, the compound is of Formula (I-I-B-1-a-1-I):

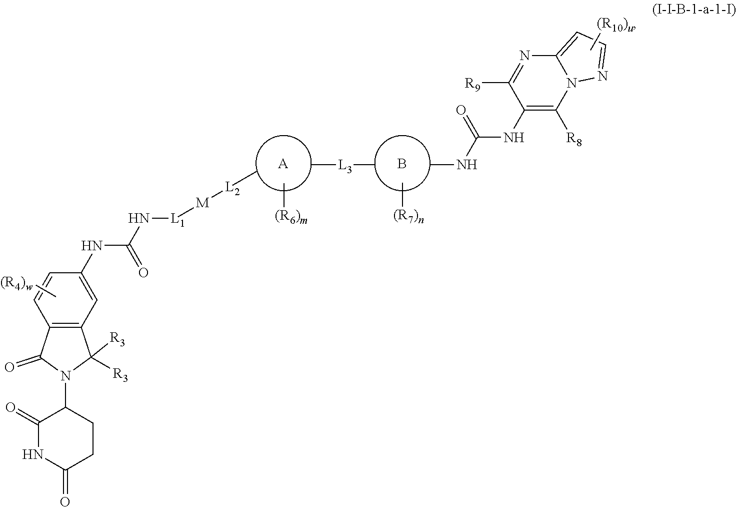

or a pharmaceutically acceptable salt, stereoisomer, solvate, prodrug, isotopic derivative, or tautomer thereof wherein all variables are as defined herein.

In some embodiments, the compound is of Formula (I-I-B-1-a-1-I-A):

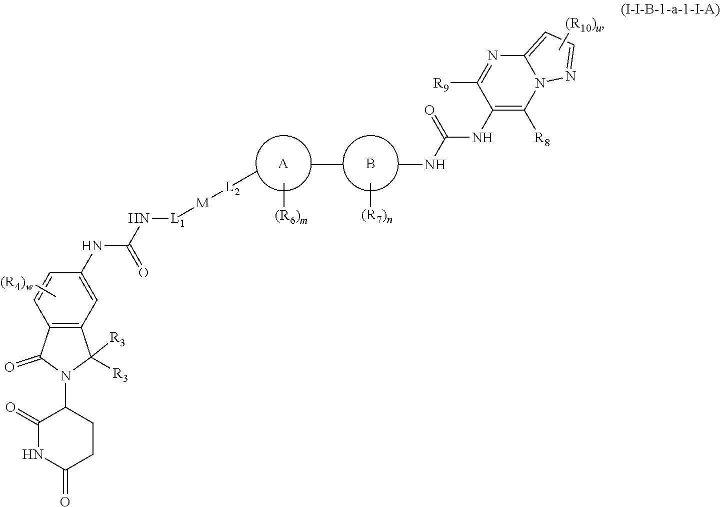

or a pharmaceutically acceptable salt, stereoisomer, solvate, prodrug, isotopic derivative, or tautomer thereof wherein all variables are as defined herein.

In some embodiments, the compound is of Formula (I-I-B-2):

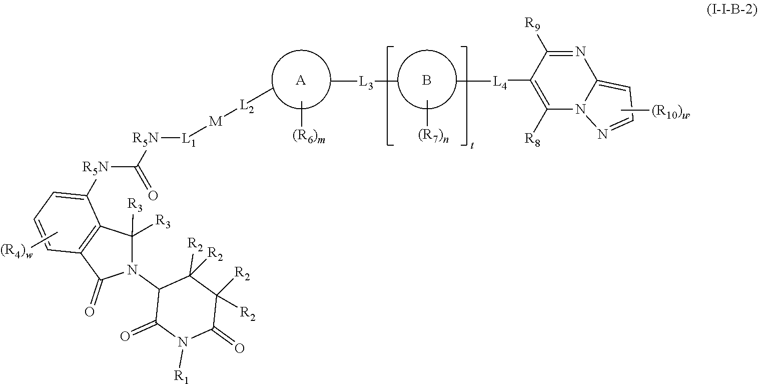

or a pharmaceutically acceptable salt, stereoisomer, solvate, prodrug, isotopic derivative, or tautomer thereof wherein all variables are as defined herein.

In some embodiments, the compound is of Formula (I-I-B-2-a):

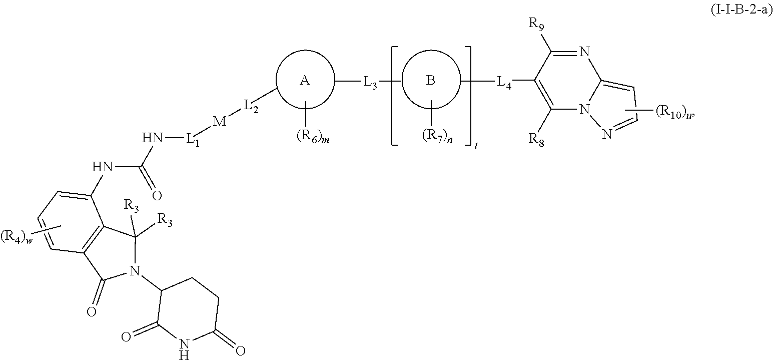

or a pharmaceutically acceptable salt, stereoisomer, solvate, prodrug, isotopic derivative, or tautomer thereof wherein all variables are as defined herein.

In some embodiments, the compound is of Formula (I-I-B-2-a-1):

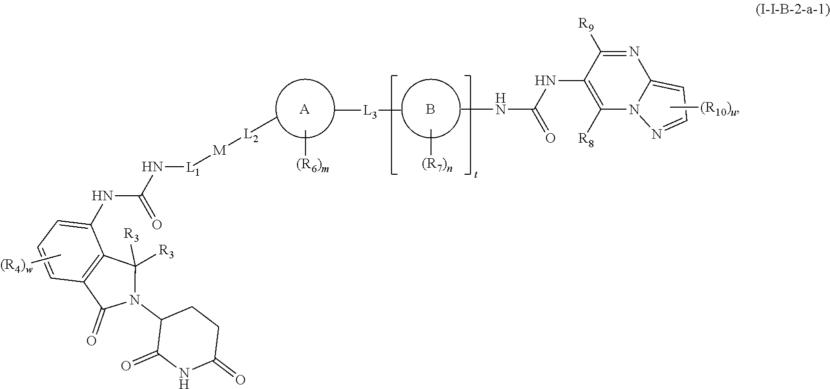

or a pharmaceutically acceptable salt, stereoisomer, solvate, prodrug, isotopic derivative, or tautomer thereof wherein all variables are as defined herein.

In some embodiments, the compound is of Formula (I-I-B-2-a-1-I):

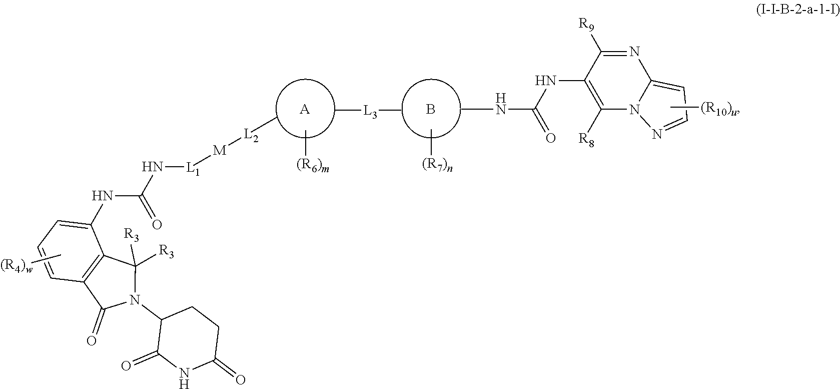

or a pharmaceutically acceptable salt, stereoisomer, solvate, prodrug, isotopic derivative, or tautomer thereof wherein all variables are as defined herein.

In some embodiments, the compound is of Formula (I-I-B-2-a-1-I-A):

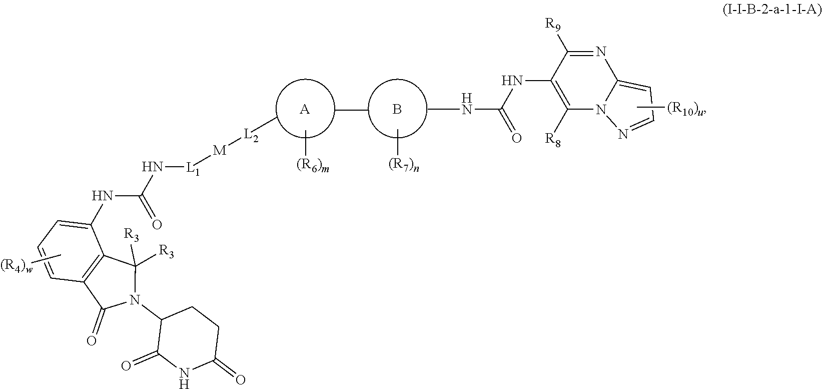

or a pharmaceutically acceptable salt, stereoisomer, solvate, prodrug, isotopic derivative, or tautomer thereof wherein all variables are as defined herein.

In some embodiments, the compound is of Formula (I-I-B-2-a-1-I-A-1):

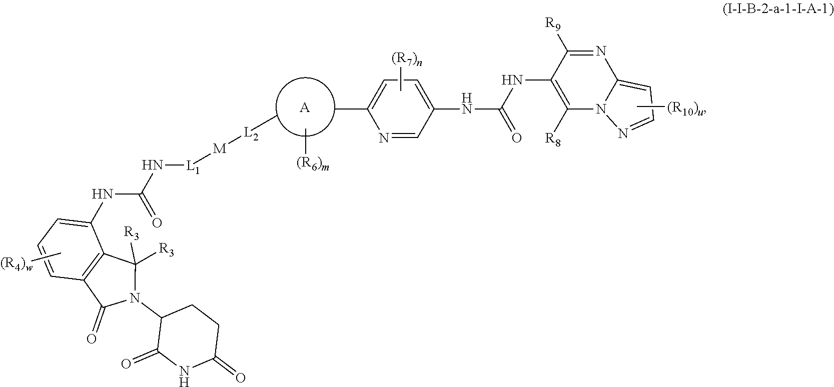

or a pharmaceutically acceptable salt, stereoisomer, solvate, prodrug, isotopic derivative, or tautomer thereof, wherein n is selected from 0 and 1, and all other variables are as defined herein.

In some embodiments, the compound is of Formula (I-I-B-2-a-1-I-A-1-I):

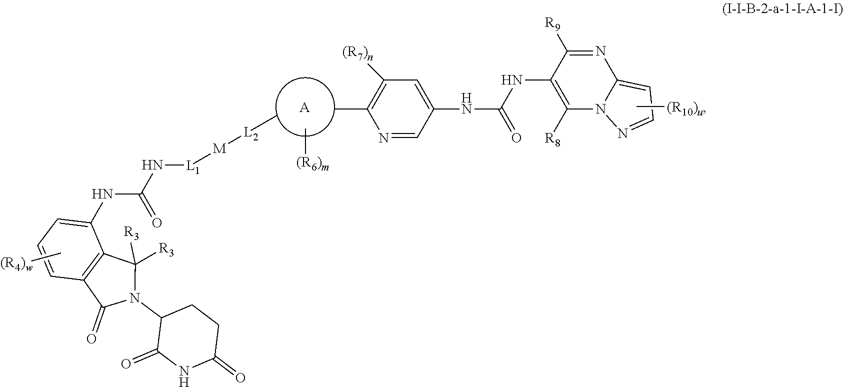

or a pharmaceutically acceptable salt, stereoisomer, solvate, prodrug, isotopic derivative, or tautomer thereof, wherein n is selected from 0 and 1; and all other variables are as defined herein.

In some embodiments, the compound is of Formula (I-I-B-2-a-1-I-A-1-I-C):

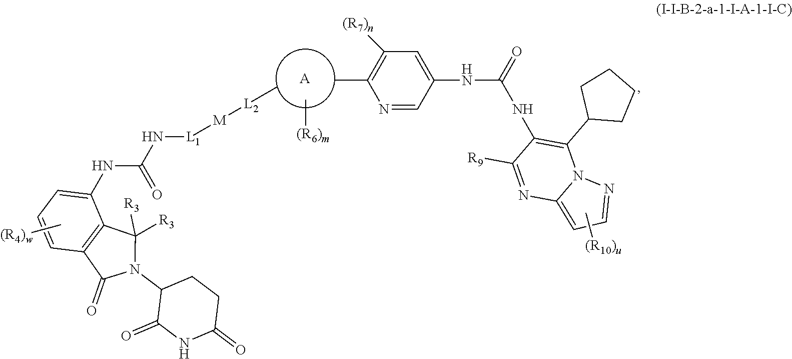

or a pharmaceutically acceptable salt, stereoisomer, solvate, prodrug, isotopic derivative, or tautomer thereof, wherein n is selected from 0 and 1 and all other variables are as defined herein.

In some embodiments, the compound is of Formula (I-I-B-2-a-1-I-A-1-I-C-3):

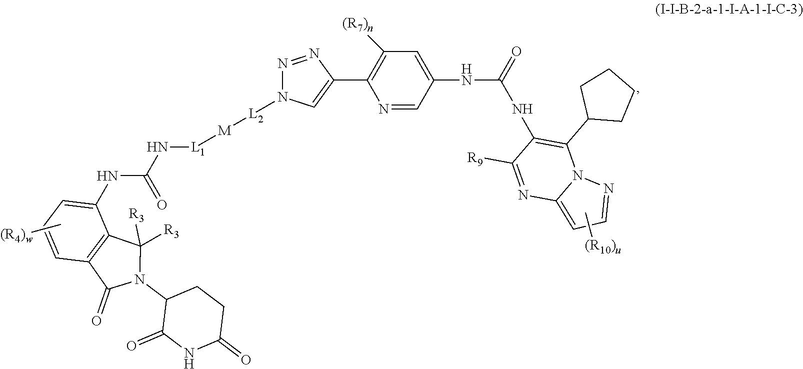

or a pharmaceutically acceptable salt, stereoisomer, solvate, prodrug, isotopic derivative, or tautomer thereof, wherein n is selected from 0 and 1; each R₃ is H or two R₃ are ═O; and all other variables are as defined herein.

In some embodiments, the compound is of Formula (I-I-B-2-a-1-I-A-1-I-C-3-a):

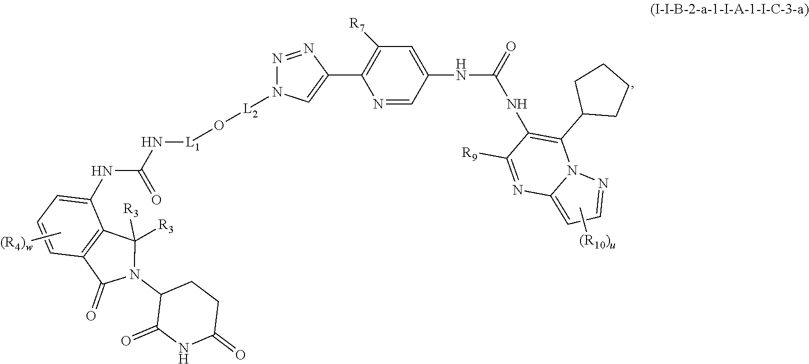

or a pharmaceutically acceptable salt, stereoisomer, solvate, prodrug, isotopic derivative, or tautomer thereof, wherein each R₃ is H or two R₃ are ═O; and all other variables are as defined herein.

In some embodiments, the compound is of Formula (I-I-B-2-a-1-I-A-1-I-C-3-a-H):

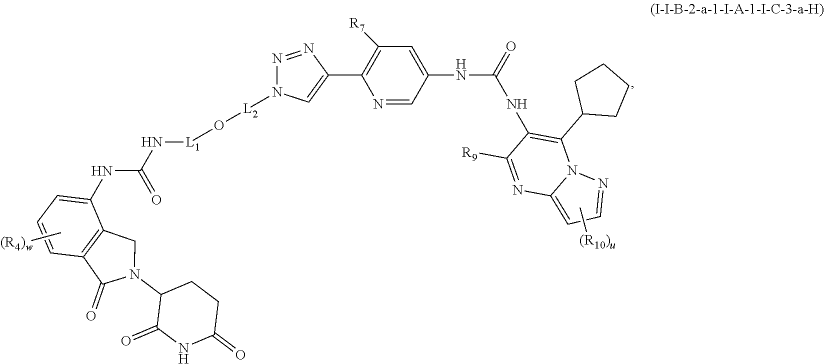

or a pharmaceutically acceptable salt, stereoisomer, solvate, prodrug, isotopic derivative, or tautomer thereof, wherein all variables are as defined herein.

In some embodiments, the compound is of Formula (I-I-B-2-a-1-I-A-1-I-C-3-a-O):

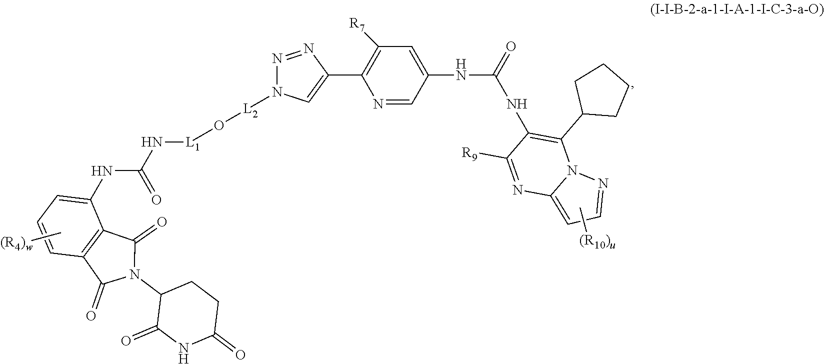

or a pharmaceutically acceptable salt, stereoisomer, solvate, prodrug, isotopic derivative, or tautomer thereof, wherein all variables are as defined herein.

In some embodiments, the compound is of Formula (I-II-A):

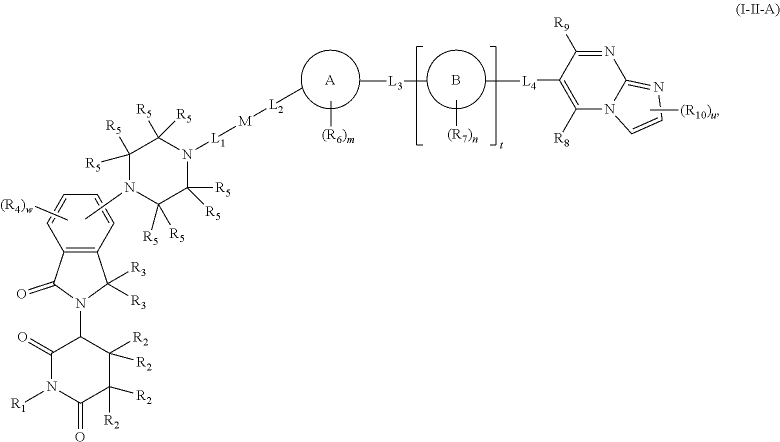

or a pharmaceutically acceptable salt, stereoisomer, solvate, prodrug, isotopic derivative, or tautomer thereof wherein all variables are as defined herein.

In some embodiments, the compound is of Formula (I-II-A-1):

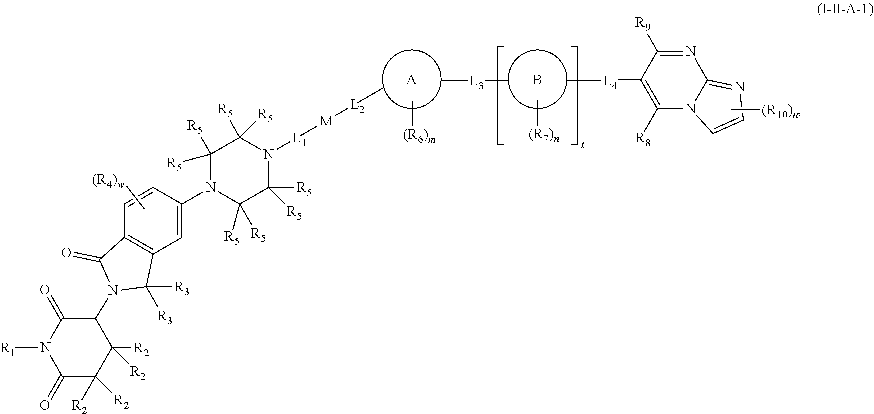

or a pharmaceutically acceptable salt, stereoisomer, solvate, prodrug, isotopic derivative, or tautomer thereof wherein all variables are as defined herein.

In some embodiments, the compound is of Formula (I-II-A-1-a):

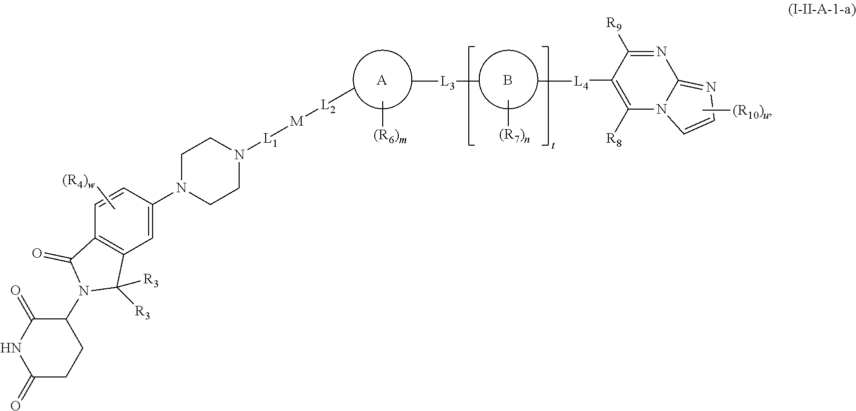

or a pharmaceutically acceptable salt, stereoisomer, solvate, prodrug, isotopic derivative, or tautomer thereof wherein all variables are as defined herein.

In some embodiments, the compound is of Formula (I-II-A-1-a-1):

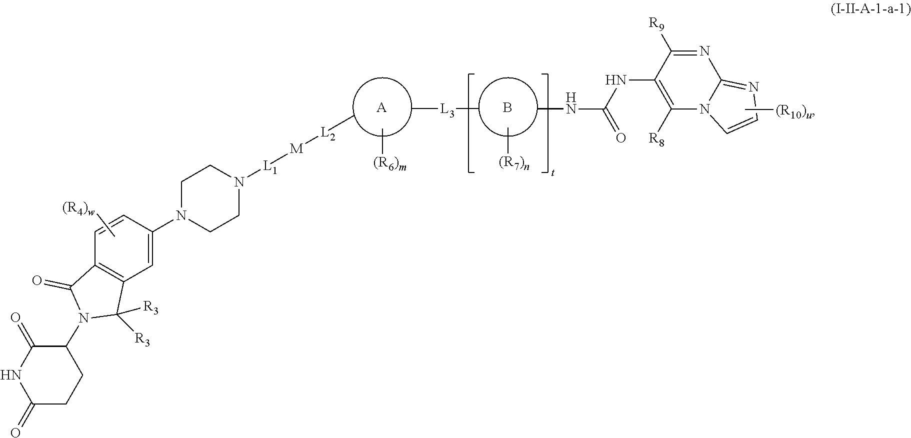

or a pharmaceutically acceptable salt, stereoisomer, solvate, prodrug, isotopic derivative, or tautomer thereof wherein all variables are as defined herein.

In some embodiments, the compound is of Formula (I-II-A-1-a-1-I):

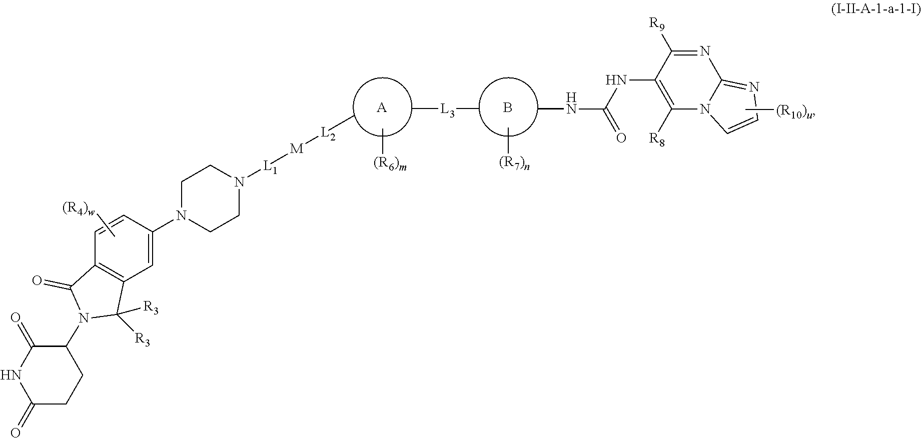

or a pharmaceutically acceptable salt, stereoisomer, solvate, prodrug, isotopic derivative, or tautomer thereof wherein all variables are as defined herein.

In some embodiments, the compound is of Formula (I-II-A-1-a-1-I-A):

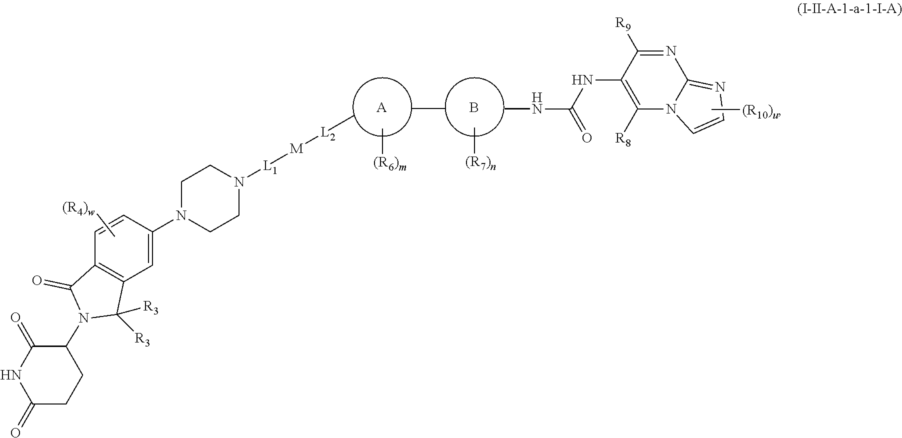

or a pharmaceutically acceptable salt, stereoisomer, solvate, prodrug, isotopic derivative, or tautomer thereof wherein all variables are as defined herein.

In some embodiments, the compound is of Formula (I-II-A-1-a-1-I-A-1):

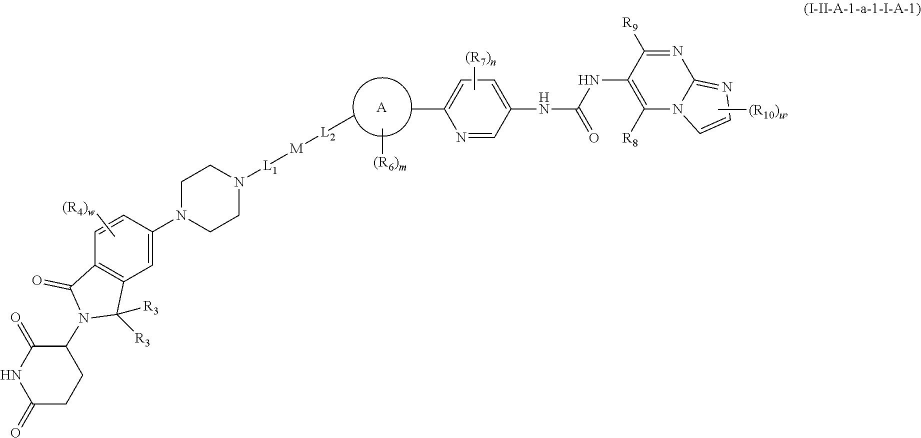

or a pharmaceutically acceptable salt, stereoisomer, solvate, prodrug, isotopic derivative, or tautomer thereof, wherein n is 0 or 1, and all other variables are as defined herein.

In some embodiments, the compound is of Formula (I-II-A-1-a-1-I-A-1-I):

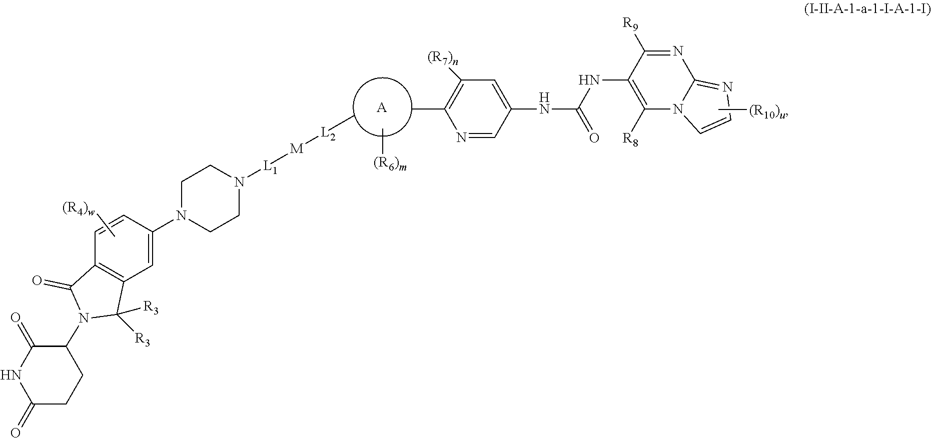

or a pharmaceutically acceptable salt, stereoisomer, solvate, prodrug, isotopic derivative, or tautomer thereof, wherein n is 0 or 1, and all other variables are as defined herein.

In some embodiments, the compound is of Formula (I-II-A-1-a-1-I-A-1-I-C):

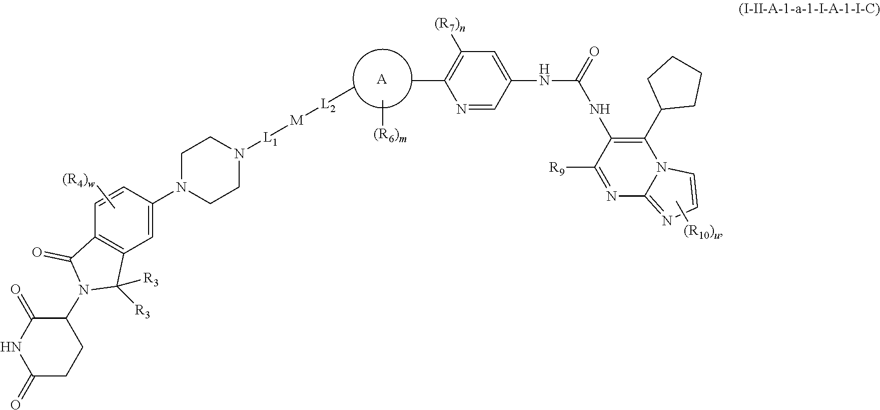

or a pharmaceutically acceptable salt, stereoisomer, solvate, prodrug, isotopic derivative, or tautomer thereof, wherein n is 0 or 1, and all other variables are as defined herein.

In some embodiments, the compound is of Formula (I-II-A-1-a-1-I-A-1-I-C-1):

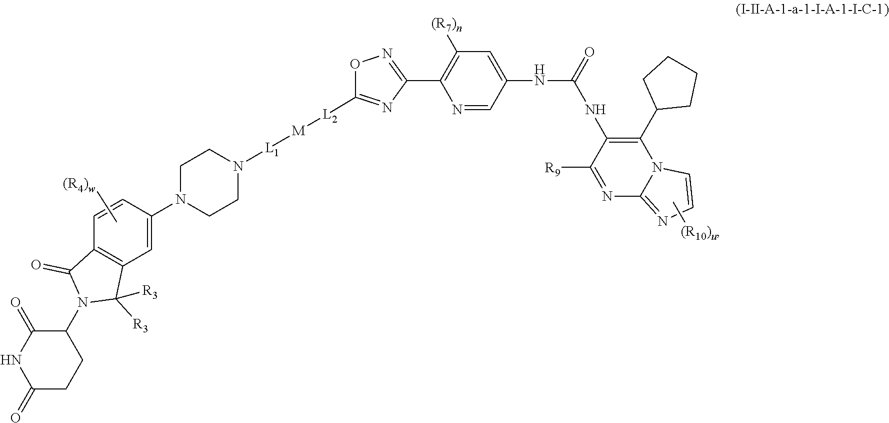

or a pharmaceutically acceptable salt, stereoisomer, solvate, prodrug, isotopic derivative, or tautomer thereof, wherein n is 0 or 1; each R₃ is H or two R₃ are ═O; and all other variables are as defined herein.

In some embodiments, the compound is of Formula (I-II-A-1-a-1-I-A-1-I-C-1-a):

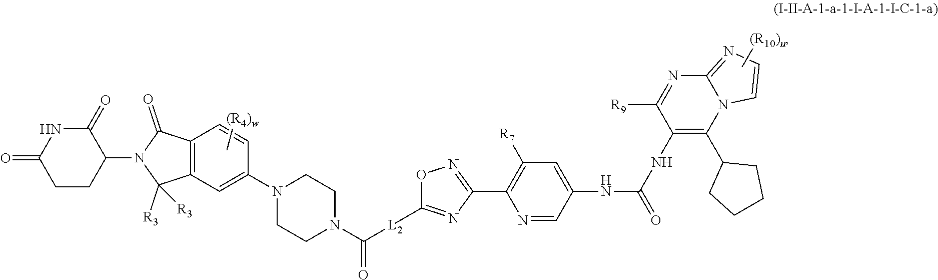

or a pharmaceutically acceptable salt, stereoisomer, solvate, prodrug, isotopic derivative, or tautomer thereof, wherein each R₃ is H or two R₃ are ═O; and all other variables are as defined herein.

In some embodiments, the compound is of Formula (I-II-A-1-a-1-I-A-1-I-C-1-a-H):

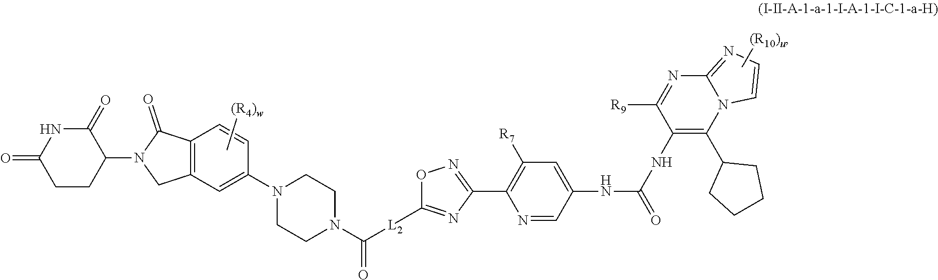

or a pharmaceutically acceptable salt, stereoisomer, solvate, prodrug, isotopic derivative, or tautomer thereof, wherein all variables are as defined herein.

In some embodiments, the compound is of Formula (I-II-A-1-a-1-I-A-1-I-C-1-a-O):

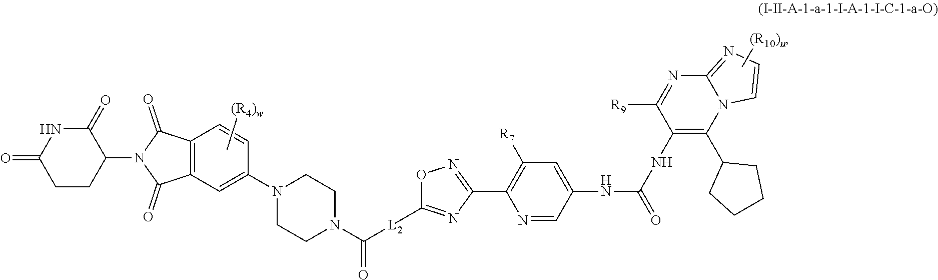

or a pharmaceutically acceptable salt, stereoisomer, solvate, prodrug, isotopic derivative, or tautomer thereof, wherein all variables are as defined herein.

In some embodiments, the compound is of Formula (I-II-A-2):

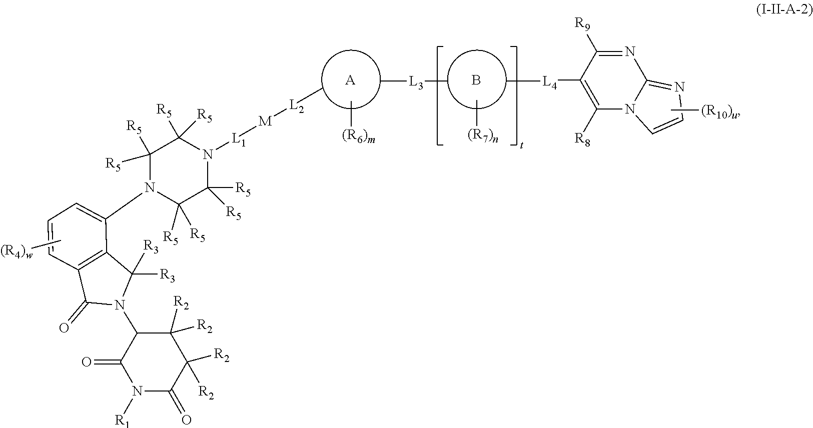

or a pharmaceutically acceptable salt, stereoisomer, solvate, prodrug, isotopic derivative, or tautomer thereof wherein all variables are as defined herein.

In some embodiments, the compound is of Formula (I-II-A-2-a):

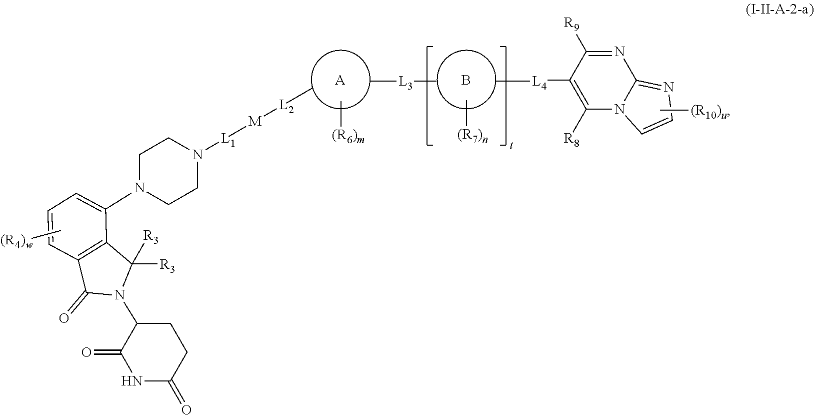

or a pharmaceutically acceptable salt, stereoisomer, solvate, prodrug, isotopic derivative, or tautomer thereof wherein all variables are as defined herein.

In some embodiments, the compound is of Formula (I-II-A-2-a-1):

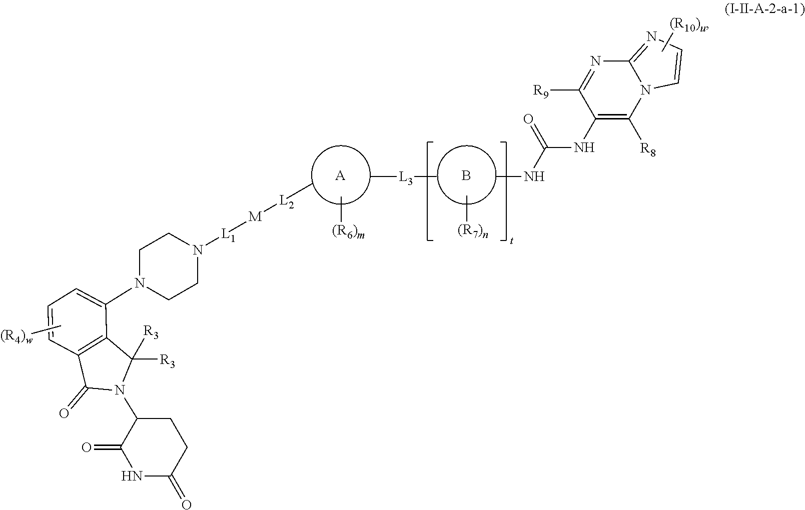

or a pharmaceutically acceptable salt, stereoisomer, solvate, prodrug, isotopic derivative, or tautomer thereof wherein all variables are as defined herein.

In some embodiments, the compound is of Formula (I-II-A-2-a-1-I):

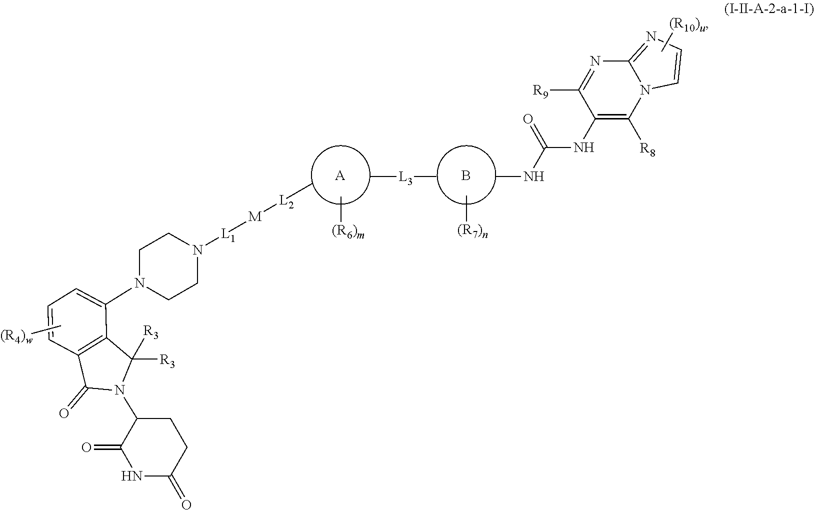

or a pharmaceutically acceptable salt, stereoisomer, solvate, prodrug, isotopic derivative, or tautomer thereof wherein all variables are as defined herein.

In some embodiments, the compound is of Formula (I-II-A-2-a-1-I-A):

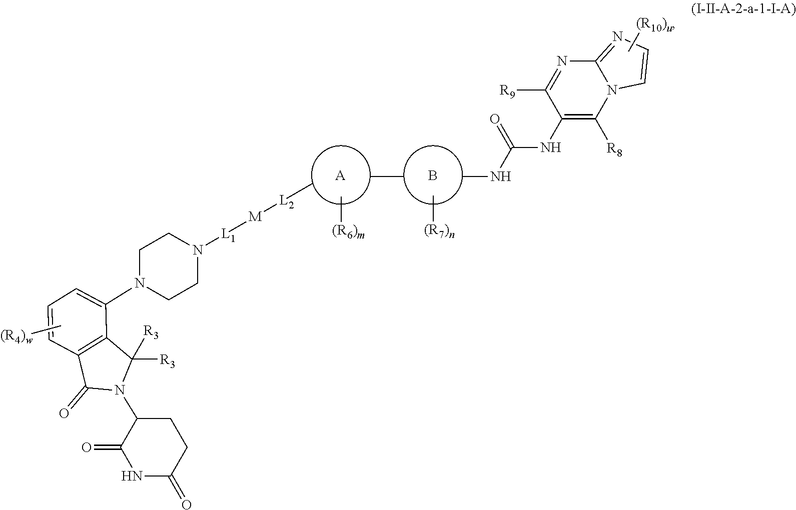

or a pharmaceutically acceptable salt, stereoisomer, solvate, prodrug, isotopic derivative, or tautomer thereof wherein all variables are as defined herein.

In some embodiments, the compound is of Formula (I-II-B):

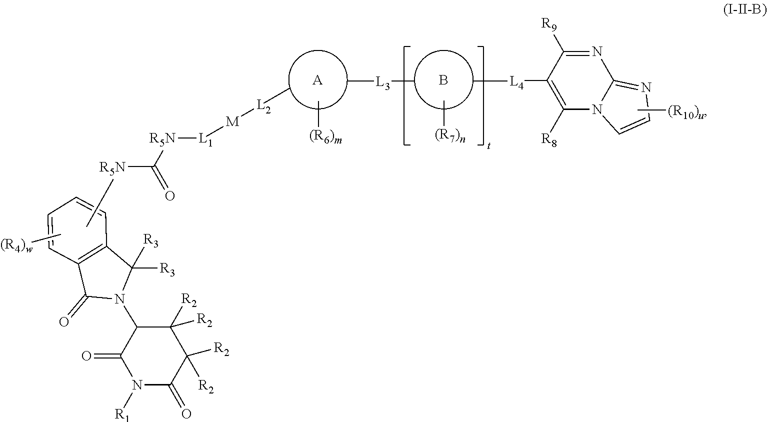

or a pharmaceutically acceptable salt, stereoisomer, solvate, prodrug, isotopic derivative, or tautomer thereof wherein all variables are as defined herein.

In some embodiments, the compound is of Formula (I-II-B-1):

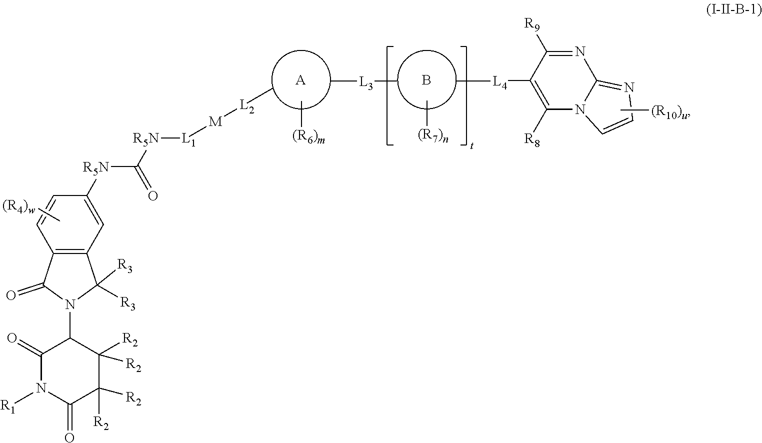

or a pharmaceutically acceptable salt, stereoisomer, solvate, prodrug, isotopic derivative, or tautomer thereof wherein all variables are as defined herein.

In some embodiments, the compound is of Formula (I-II-B-1-a):

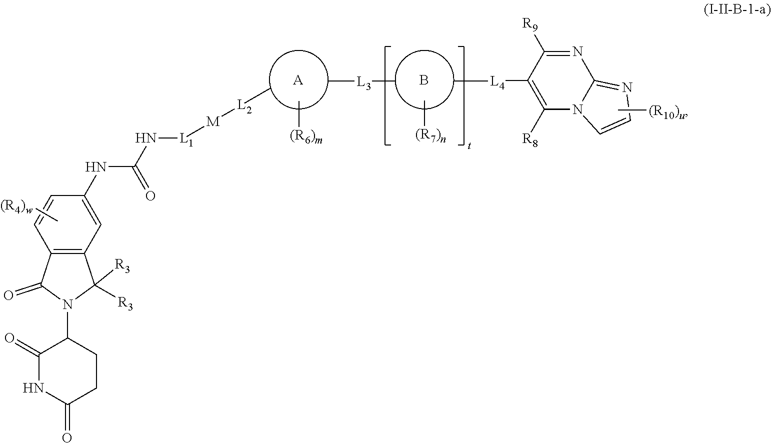

or a pharmaceutically acceptable salt, stereoisomer, solvate, prodrug, isotopic derivative, or tautomer thereof wherein all variables are as defined herein.

In some embodiments, the compound is of Formula (I-II-B-1-a-1):

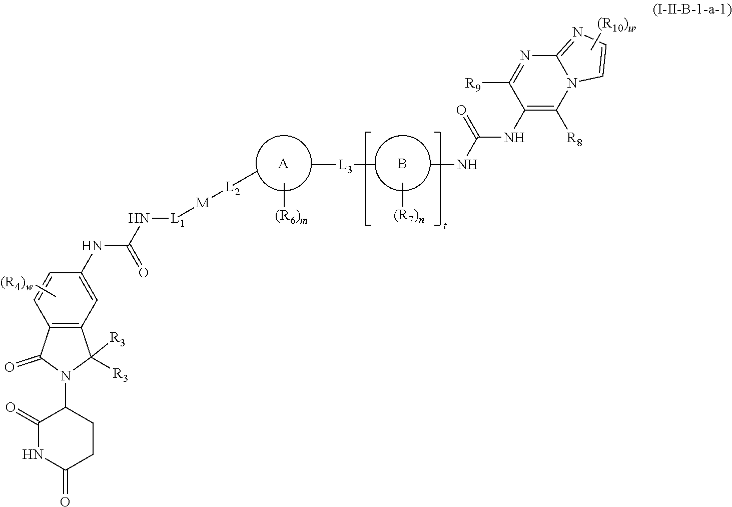

or a pharmaceutically acceptable salt, stereoisomer, solvate, prodrug, isotopic derivative, or tautomer thereof wherein all variables are as defined herein.

In some embodiments, the compound is of Formula (I-II-B-1-a-1-I):

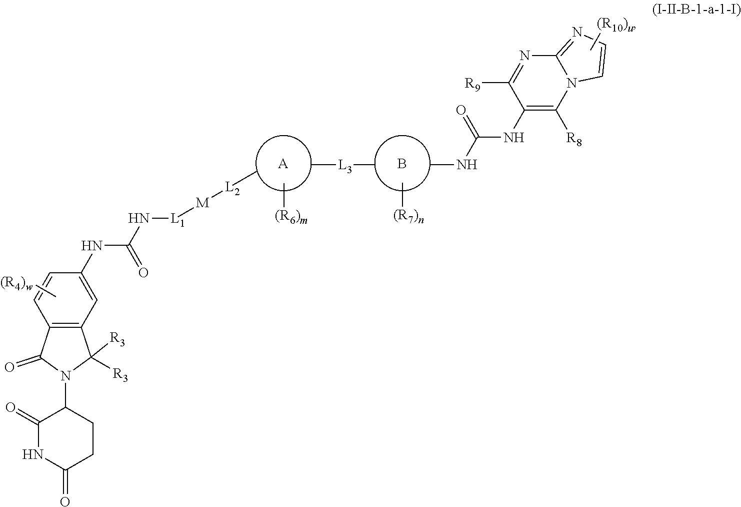

or a pharmaceutically acceptable salt, stereoisomer, solvate, prodrug, isotopic derivative, or tautomer thereof wherein all variables are as defined herein.

In some embodiments, the compound is of Formula (I-II-B-1-a-1-I-A):

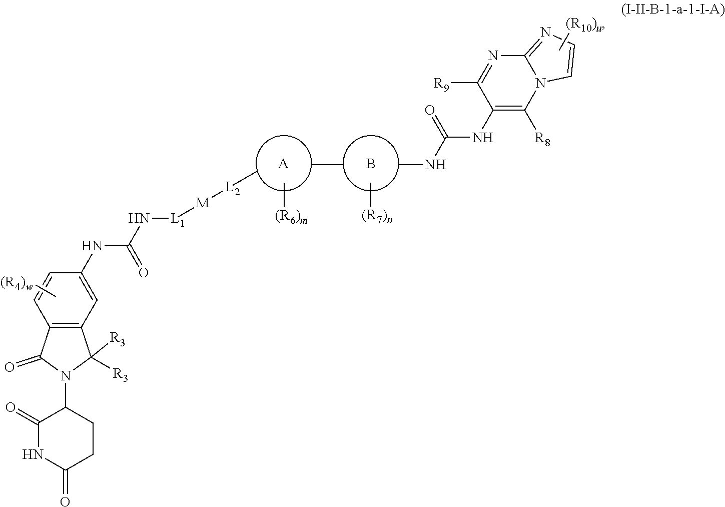

or a pharmaceutically acceptable salt, stereoisomer, solvate, prodrug, isotopic derivative, or tautomer thereof wherein all variables are as defined herein.

In some embodiments, the compound is of Formula (I-II-B-2):

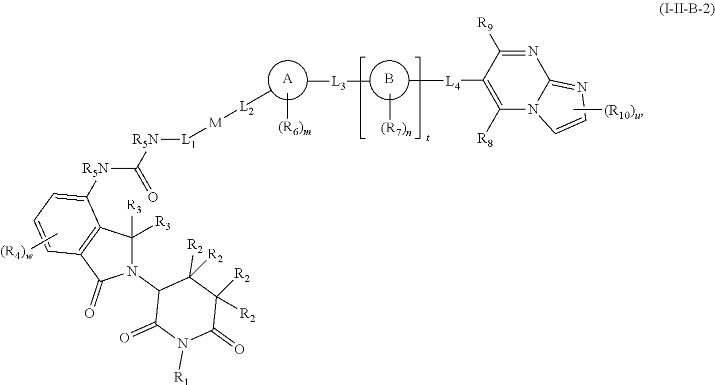

or a pharmaceutically acceptable salt, stereoisomer, solvate, prodrug, isotopic derivative, or tautomer thereof wherein all variables are as defined herein.

In some embodiments, the compound is of Formula (I-II-B-2-a):

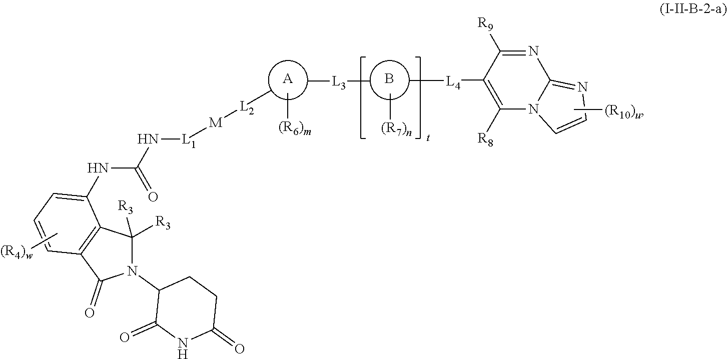

or a pharmaceutically acceptable salt, stereoisomer, solvate, prodrug, isotopic derivative, or tautomer thereof wherein all variables are as defined herein.

In some embodiments, the compound is of Formula (I-II-B-2-a-1):

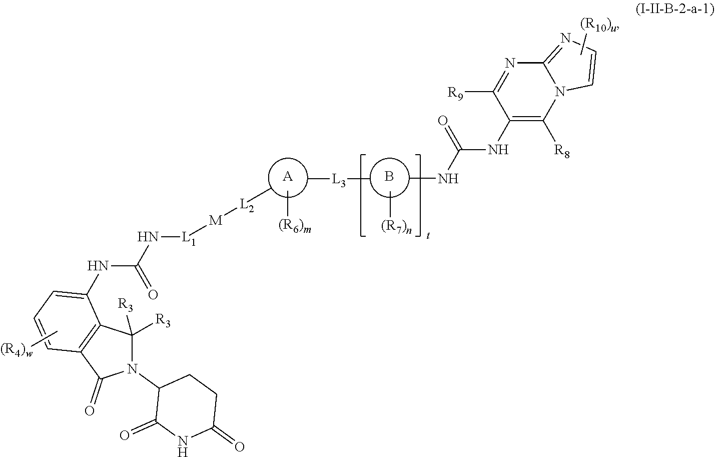

or a pharmaceutically acceptable salt, stereoisomer, solvate, prodrug, isotopic derivative, or tautomer thereof wherein all variables are as defined herein.

In some embodiments, the compound is of Formula (I-II-B-2-a-1-I):

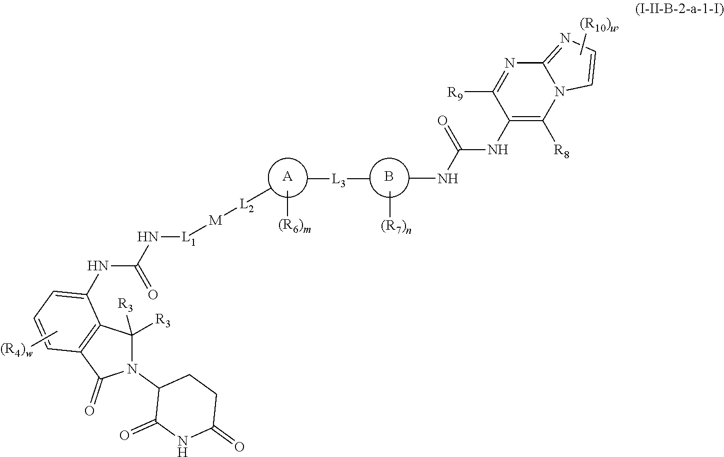

or a pharmaceutically acceptable salt, stereoisomer, solvate, prodrug, isotopic derivative, or tautomer thereof wherein all variables are as defined herein.

In some embodiments, the compound is of Formula (I-II-B-2-a-1-I-A):

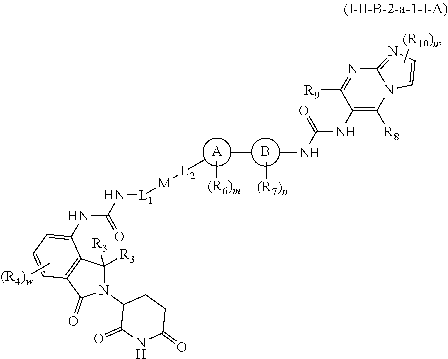

or a pharmaceutically acceptable salt, stereoisomer, solvate, prodrug, isotopic derivative, or tautomer thereof wherein all variables are as defined herein.

In some embodiments, the compound is of Formula (II-A):

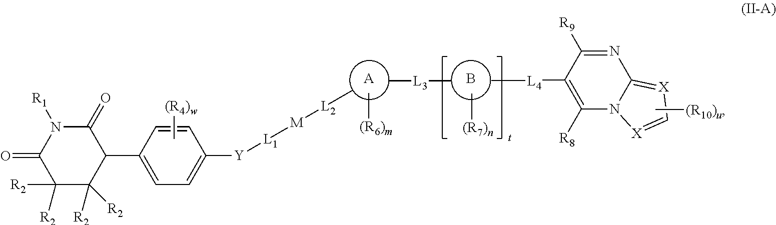

or a pharmaceutically acceptable salt, stereoisomer, solvate, prodrug, isotopic derivative, or tautomer thereof wherein all variables are as defined herein.

In some embodiments, the compound is of Formula (II-A-I):

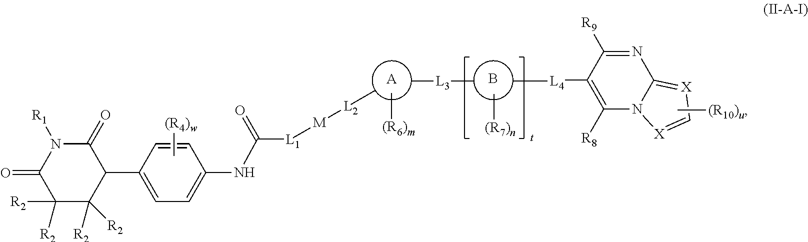

or a pharmaceutically acceptable salt, stereoisomer, solvate, prodrug, isotopic derivative, or tautomer thereof wherein all variables are as defined herein.

In some embodiments, the compound is of Formula (II-A-I-a):

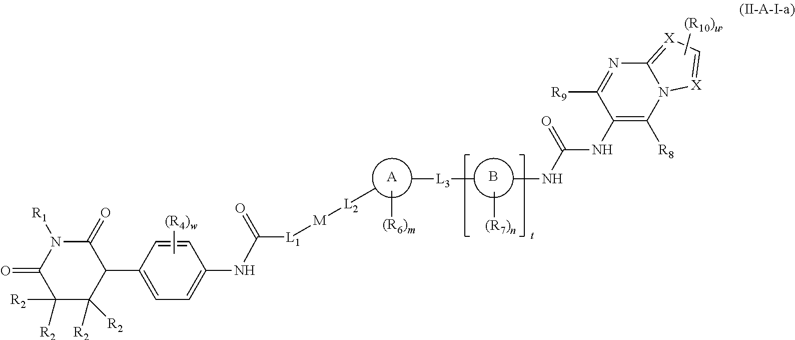

or a pharmaceutically acceptable salt, stereoisomer, solvate, prodrug, isotopic derivative, or tautomer thereof wherein all variables are as defined herein.

In some embodiments, the compound is of Formula (II-A-I-a-1):

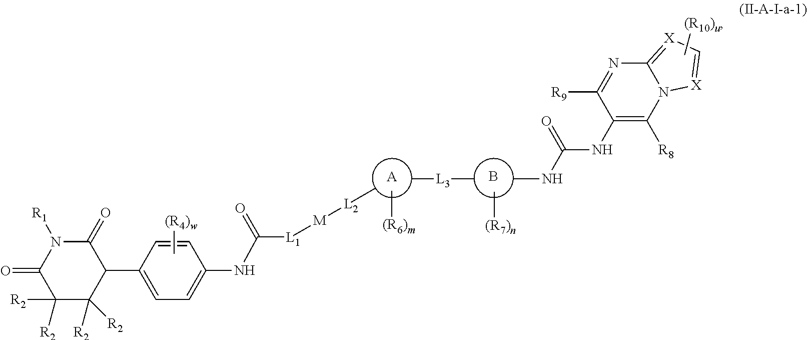

or a pharmaceutically acceptable salt, stereoisomer, solvate, prodrug, isotopic derivative, or tautomer thereof wherein all variables are as defined herein.

In some embodiments, the compound is of Formula (II-A-I-a-1-I):

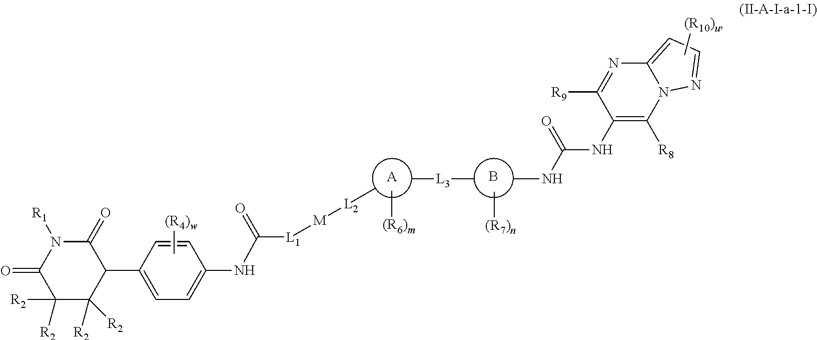

or a pharmaceutically acceptable salt, stereoisomer, solvate, prodrug, isotopic derivative, or tautomer thereof wherein all variables are as defined herein.

In some embodiments, the compound is of Formula (II-A-I-a-1-I-A):

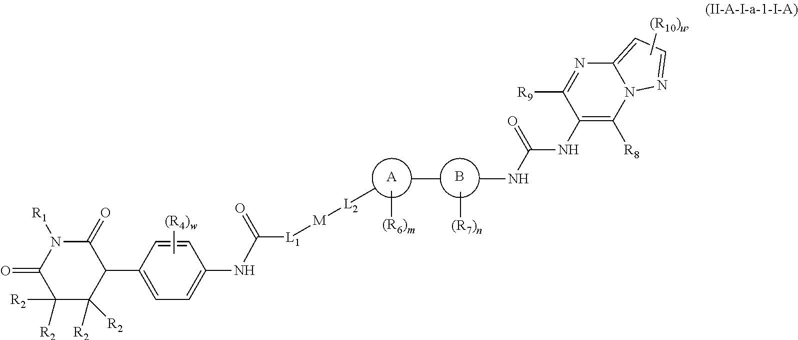

or a pharmaceutically acceptable salt, stereoisomer, solvate, prodrug, isotopic derivative, or tautomer thereof wherein all variables are as defined herein.

In some embodiments, the compound is of Formula (II-A-I-a-1-I-A-1):

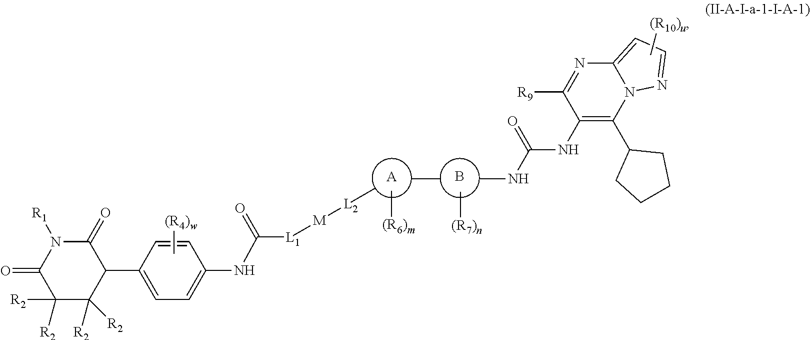

or a pharmaceutically acceptable salt, stereoisomer, solvate, prodrug, isotopic derivative, or tautomer thereof wherein all variables are as defined herein.

In some embodiments, the compound is of Formula (II-B):

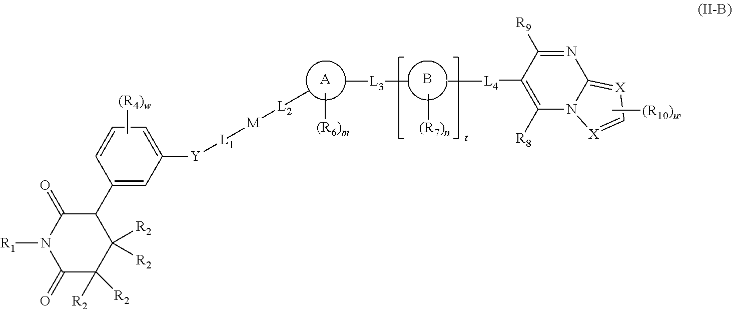

or a pharmaceutically acceptable salt, stereoisomer, solvate, prodrug, isotopic derivative, or tautomer thereof wherein all variables are as defined herein.

In some embodiments, the compound is of Formula (II-B-I):

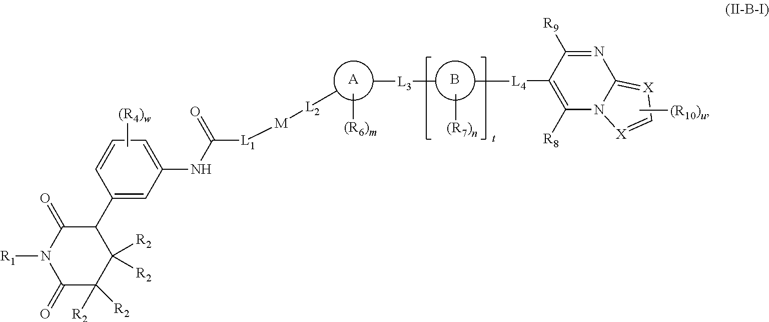

or a pharmaceutically acceptable salt, stereoisomer, solvate, prodrug, isotopic derivative, or tautomer thereof wherein all variables are as defined herein.

In some embodiments, the compound is of Formula (II-B-I-a):

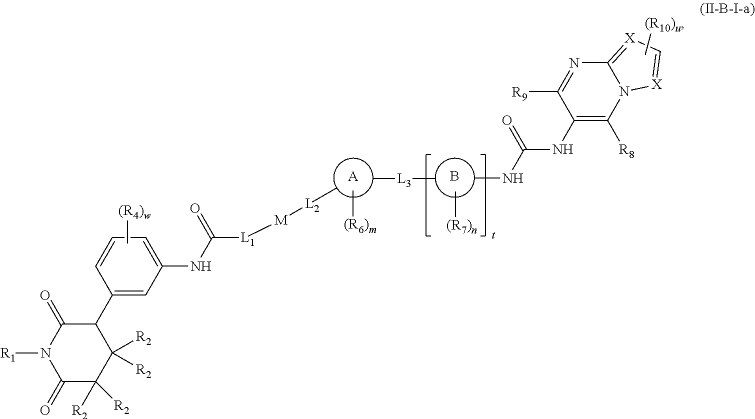

or a pharmaceutically acceptable salt, stereoisomer, solvate, prodrug, isotopic derivative, or tautomer thereof wherein all variables are as defined herein.

In some embodiments, the compound is of Formula (II-B-I-a-1):

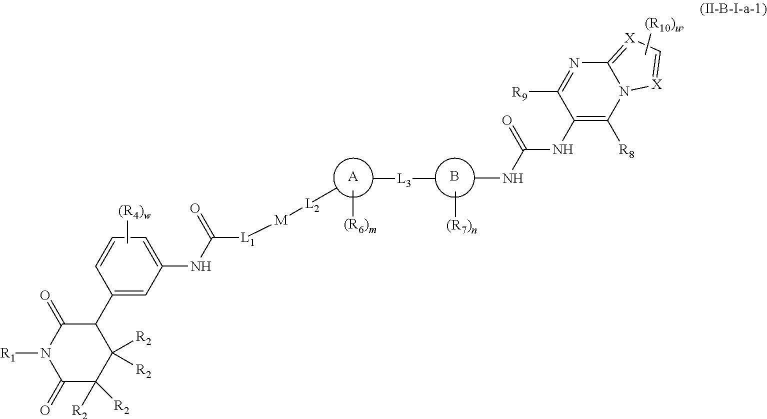

or a pharmaceutically acceptable salt, stereoisomer, solvate, prodrug, isotopic derivative, or tautomer thereof wherein all variables are as defined herein.

In some embodiments, the compound is of Formula (II-B-I-a-1-I):

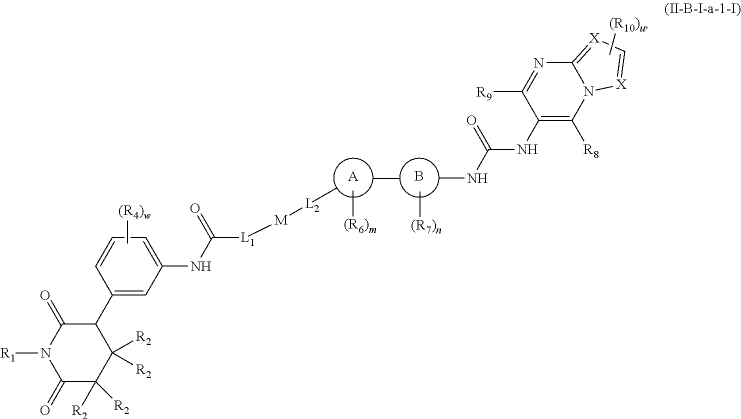

or a pharmaceutically acceptable salt, stereoisomer, solvate, prodrug, isotopic derivative, or tautomer thereof wherein all variables are as defined herein.

In some embodiments, the compound is of Formula (II-B-I-a-1-I-A):

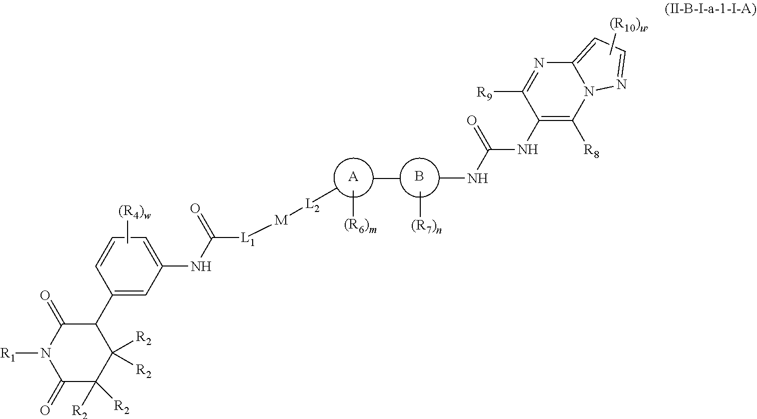

or a pharmaceutically acceptable salt, stereoisomer, solvate, prodrug, isotopic derivative, or tautomer thereof wherein all variables are as defined herein.

In some embodiments, the compound is of Formula (II-B-I-a-1-I-A-1):

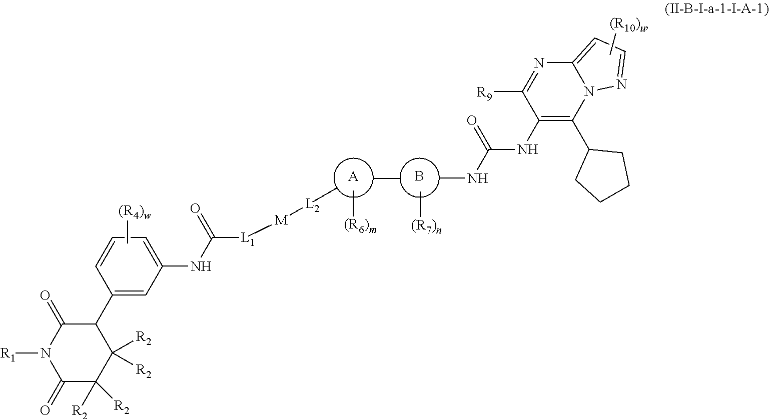

or a pharmaceutically acceptable salt, stereoisomer, solvate, prodrug, isotopic derivative, or tautomer thereof wherein all variables are as defined herein.

In some embodiments, the compound is of Formula (A-I):

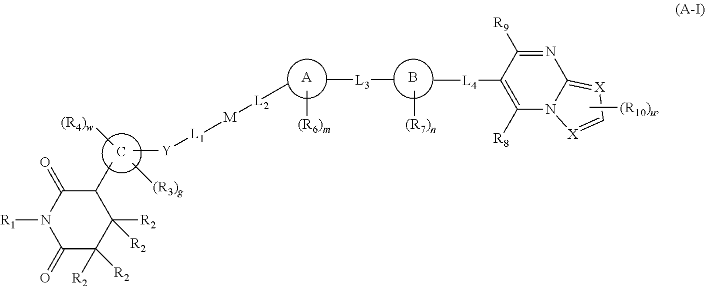

or a pharmaceutically acceptable salt, stereoisomer, solvate, prodrug, isotopic derivative, or tautomer thereof wherein all variables are as defined herein.

In some embodiments, the compound is of Formula (A-I-1):

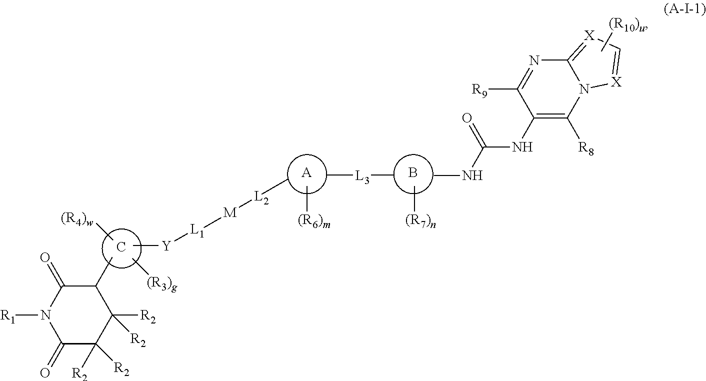

or a pharmaceutically acceptable salt, stereoisomer, solvate, prodrug, isotopic derivative, or tautomer thereof wherein all variables are as defined herein.

In some embodiments, the compound is of Formula (A-I-1-a):

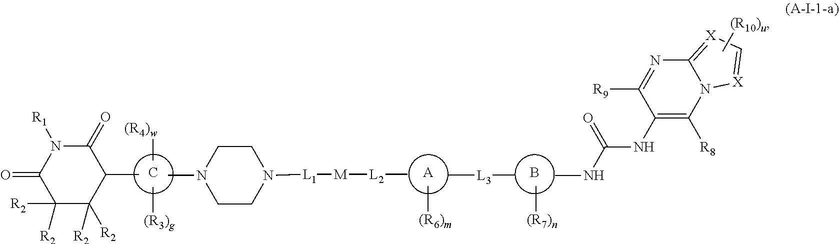

or a pharmaceutically acceptable salt, stereoisomer, solvate, prodrug, isotopic derivative, or tautomer thereof wherein all variables are as defined herein.

In some embodiments, the compound is of Formula (A-I-1-a-I):

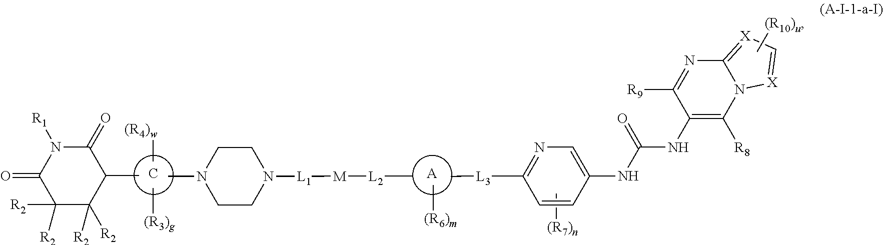

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A):

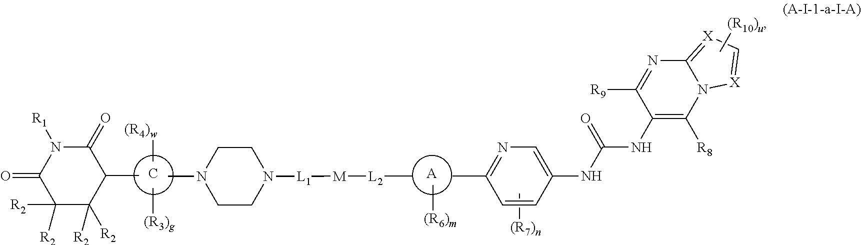

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1):

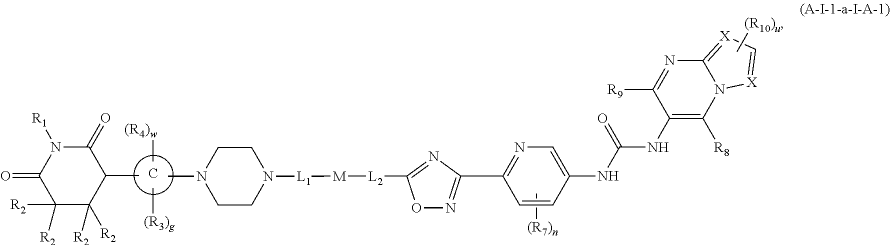

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-a):

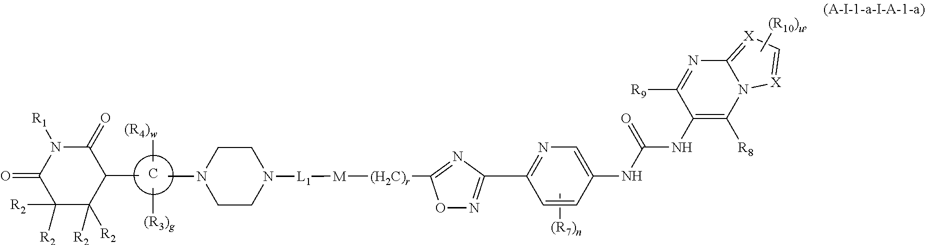

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1):

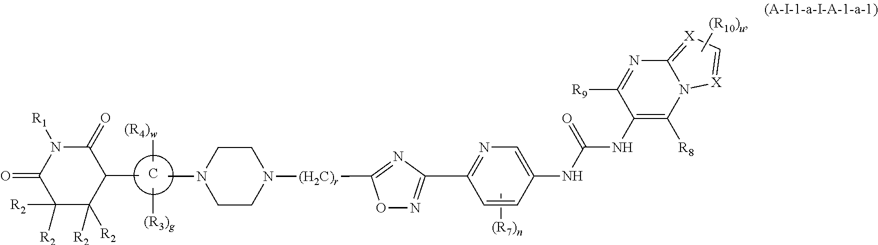

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1-a):

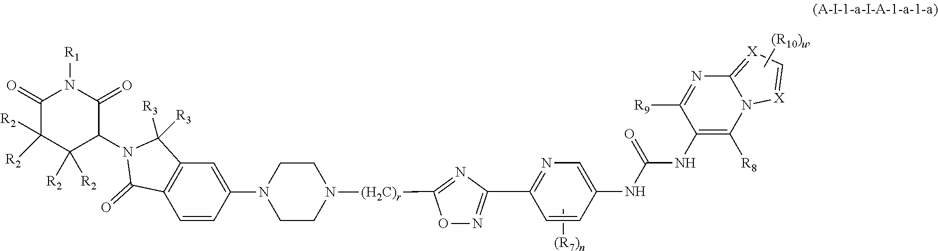

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1-a-1):

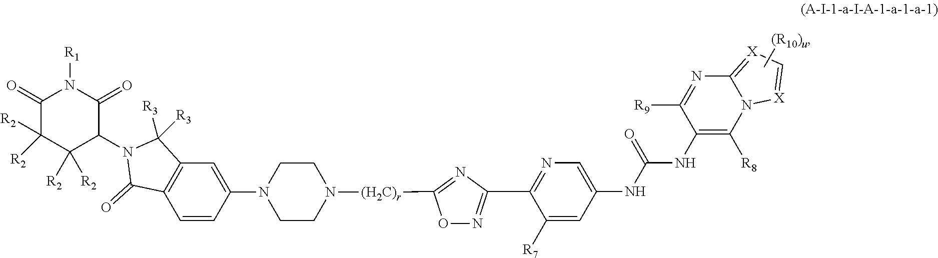

or a pharmaceutically acceptable salt, stereoisomer, solvate, prodrug, isotopic derivative, or tautomer thereof wherein r is an integer selected from 1, 2, 3, 4, 5 and 6; R₇ is selected from F, Cl, Br, I, CN, SO₂CH₃; and all other variables are as defined herein.

In some embodiments, the compound is of Formula (A-I-1-a-I-A-1-a-1-a-1-a):

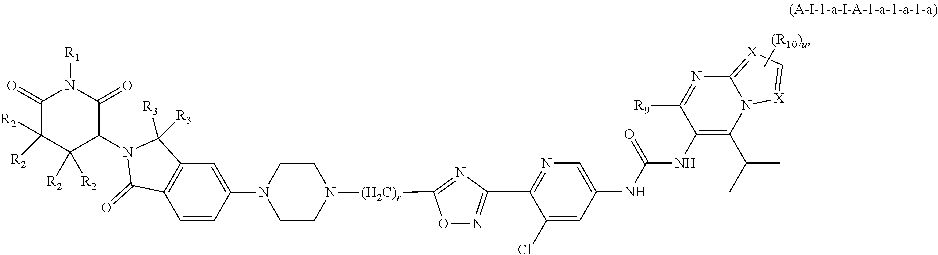

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1-a-1-a-1):

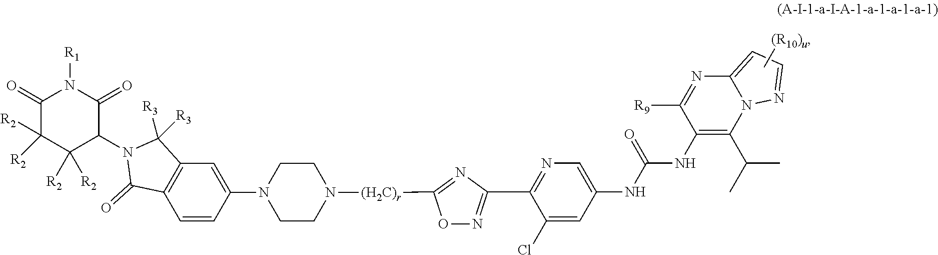

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1-a-1-a-2):

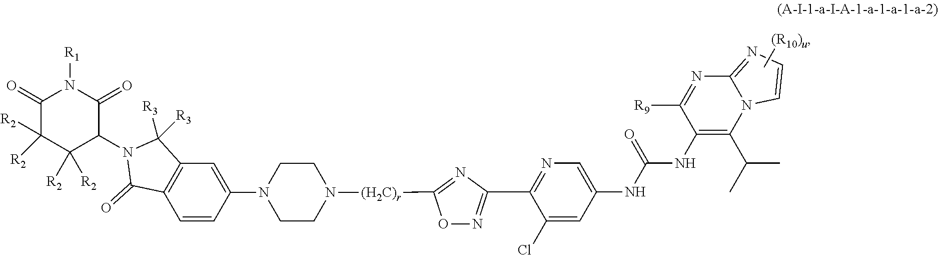

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1-a-1-a-3):

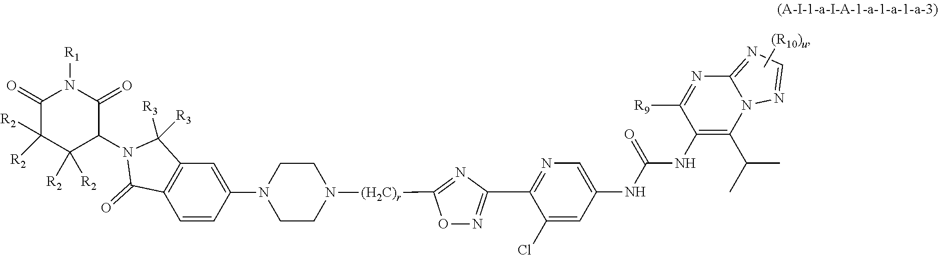

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1-a-1-b):

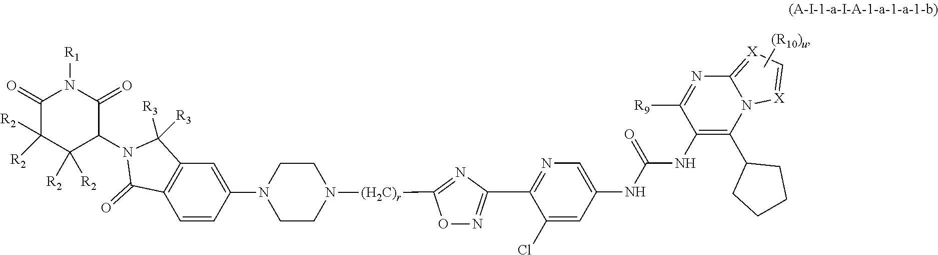

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1-a-1-b-1):

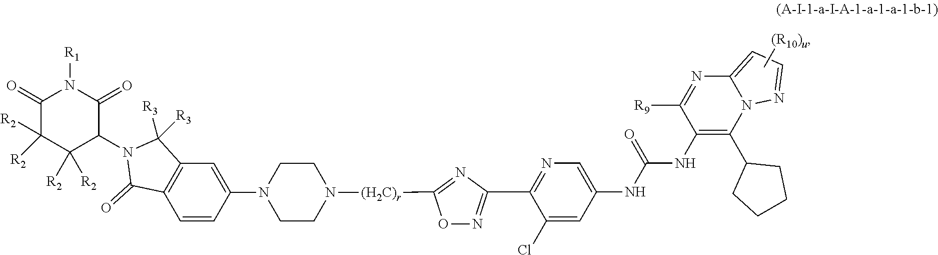

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1-a-1-b-2):

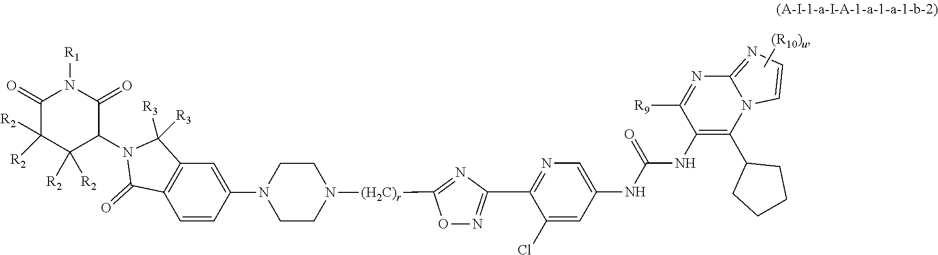

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-1-a-1-b-3):

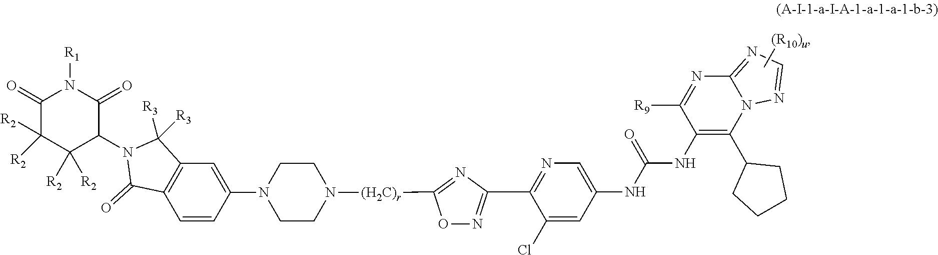

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2):

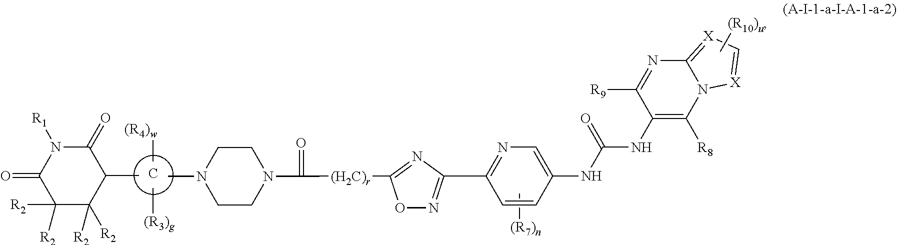

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2-a):

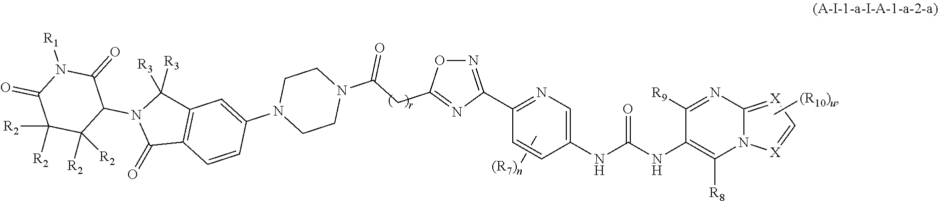

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2-a-1):

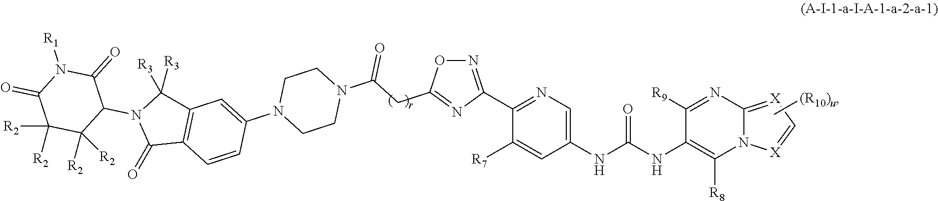

or a pharmaceutically acceptable salt, stereoisomer, solvate, prodrug, isotopic derivative, or tautomer thereof wherein r is an integer selected from 1, 2, 3, 4, 5 and 6; R₇ is selected from F, Cl, Br, I, CN, SO₂CH₃; and all other variables are as defined herein.

In some embodiments, the compound is of Formula (A-I-1-a-I-A-1-a-2-a-1-a):

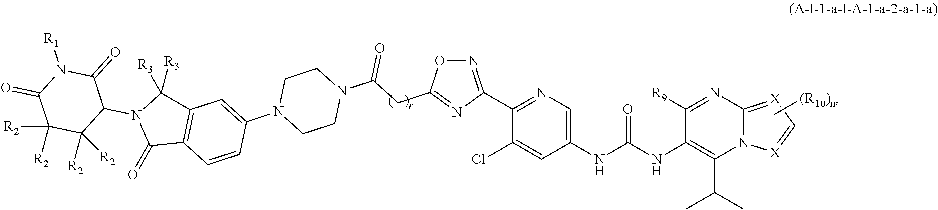

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2-a-1-a-1):

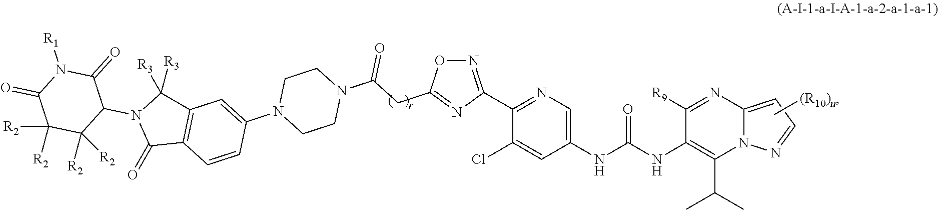

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2-a-1-a-2):

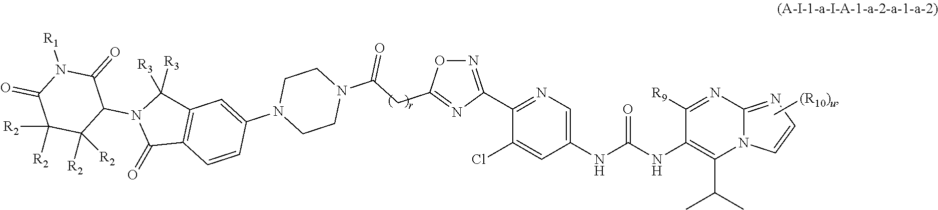

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2-a-1-a-3):

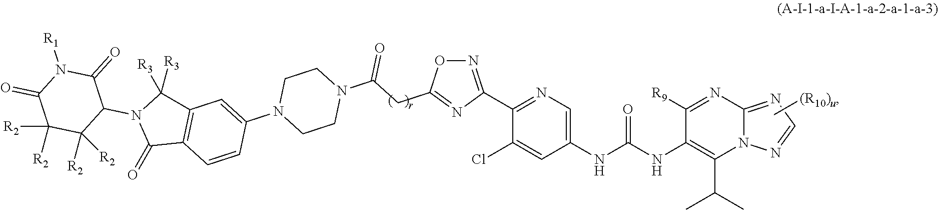

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2-a-1-b):

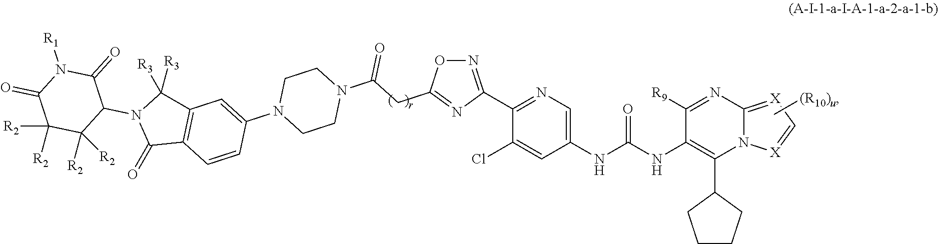

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2-a-1-b-1):

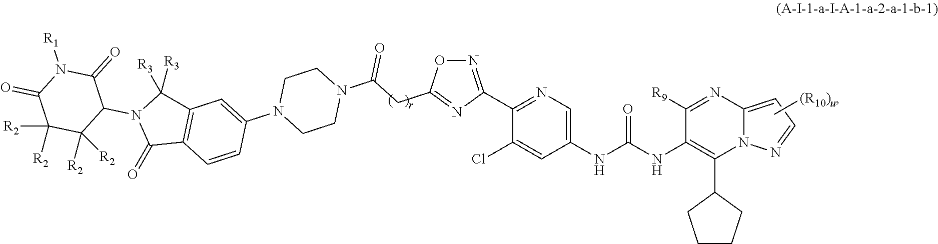

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2-a-1-b-2):

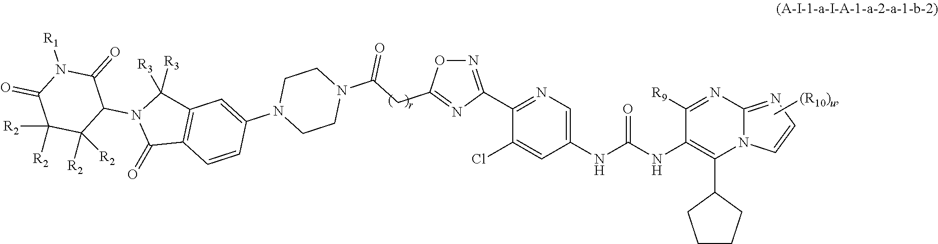

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-2-a-1-b-3):

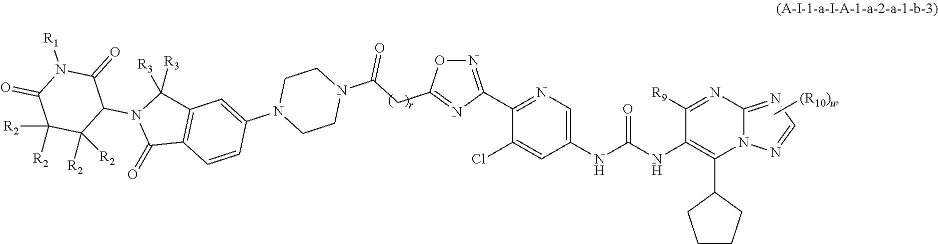

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-a-3):

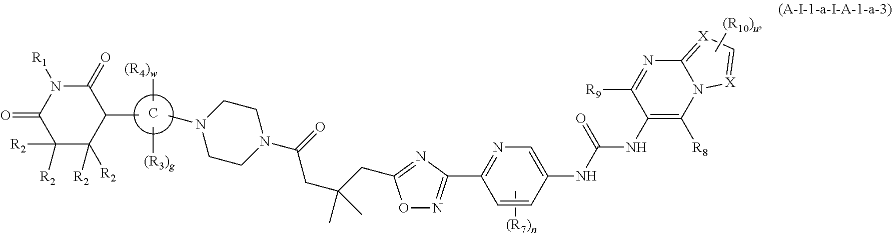

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-1-b):

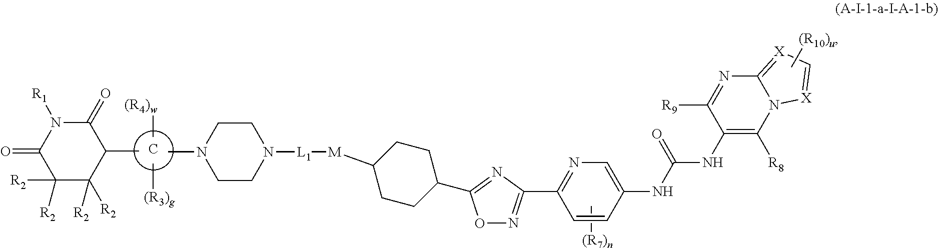

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-1):

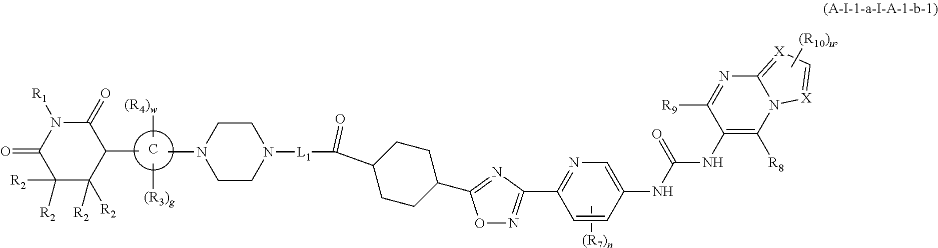

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-1-a):

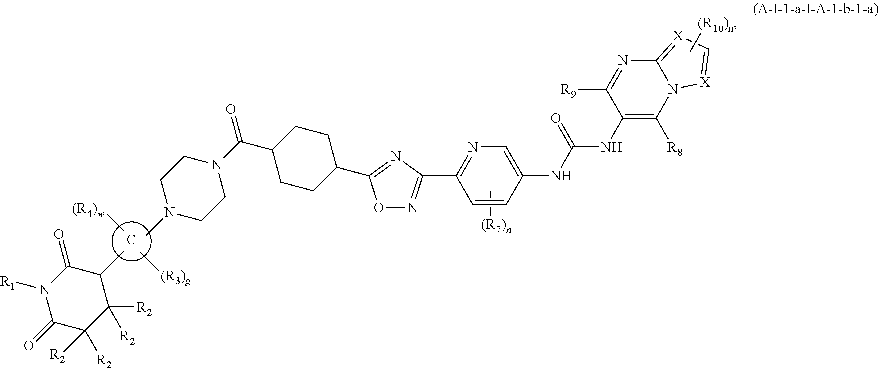

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-1-a*):

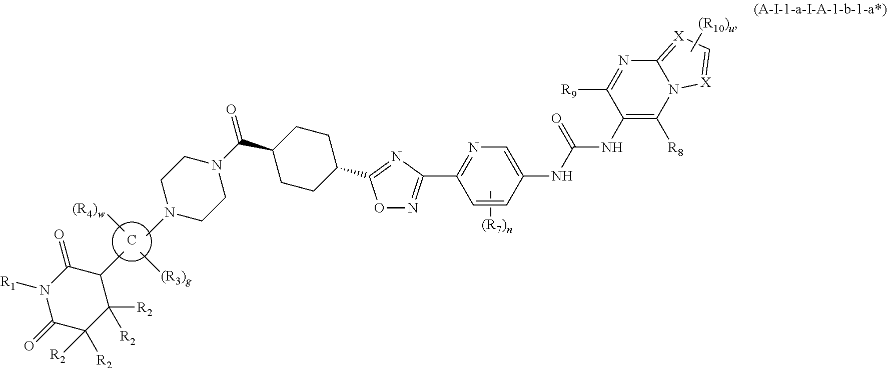

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-1-a**):

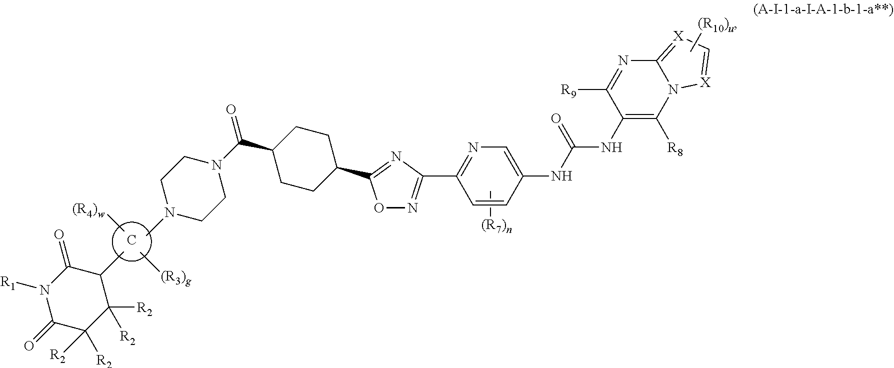

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-1-a***):

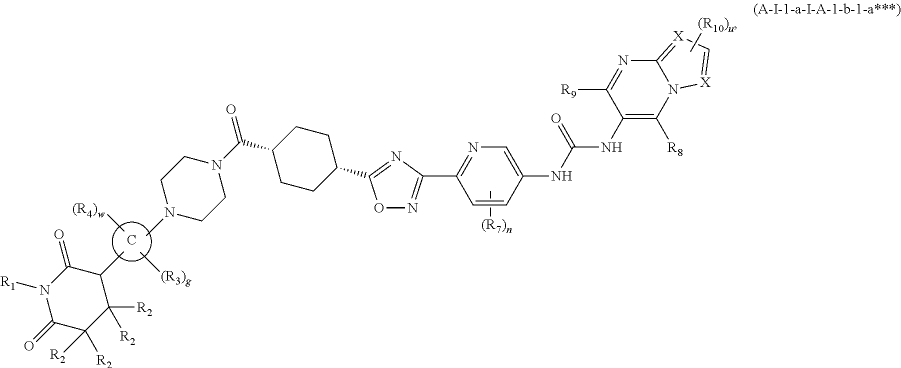

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-1-a****):

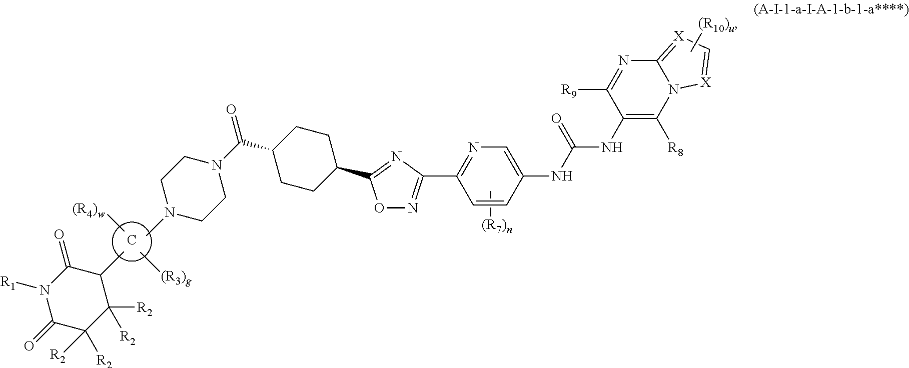

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2):

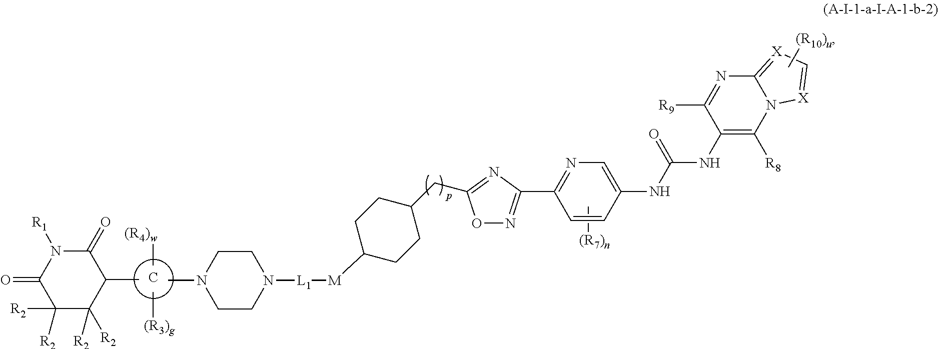

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a):

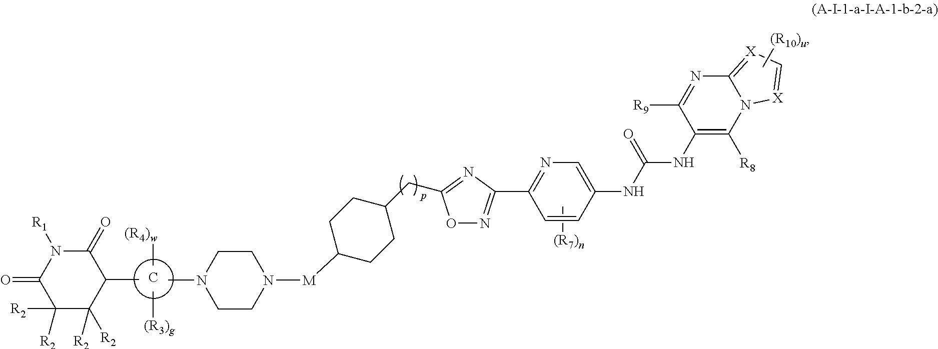

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-1):

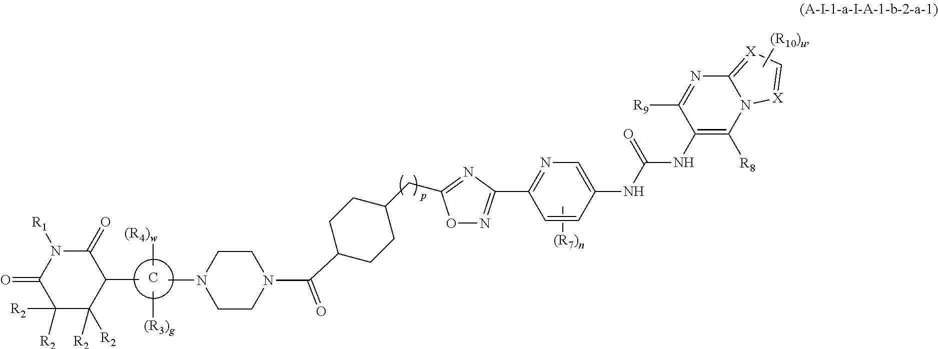

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-1*):

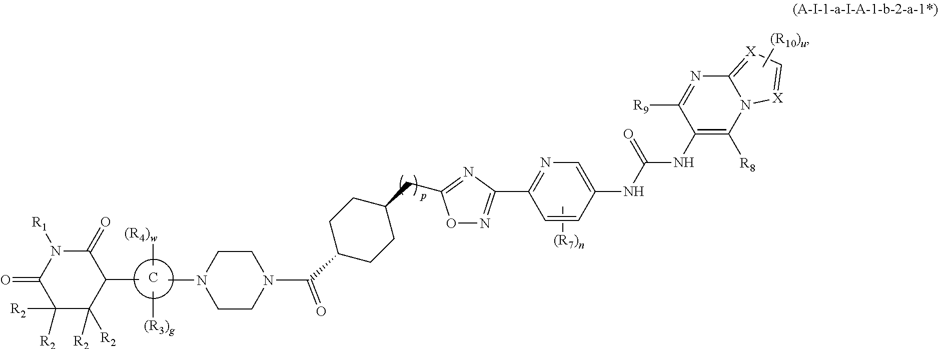

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-1**):

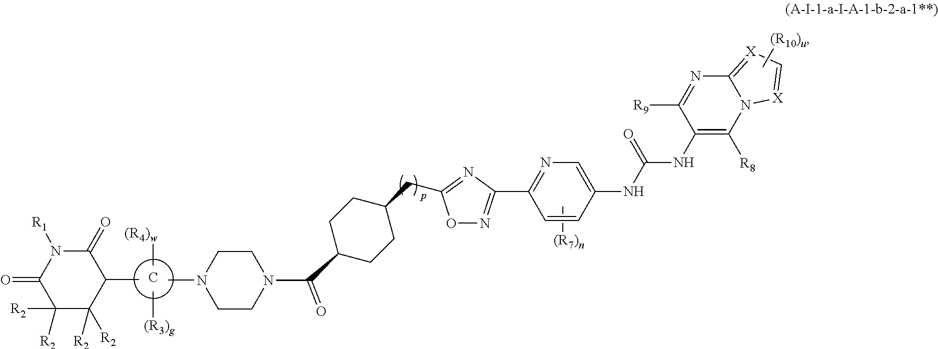

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-1

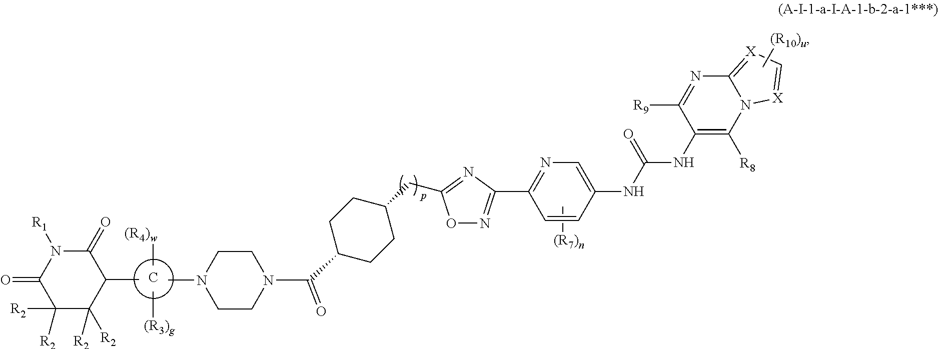

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-1****):

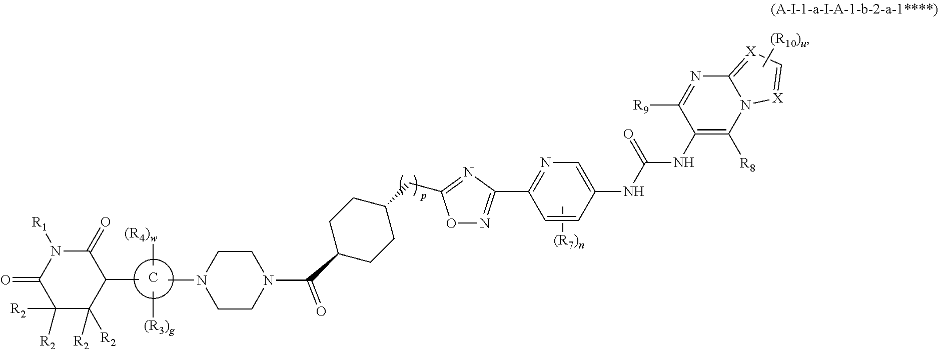

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-2):

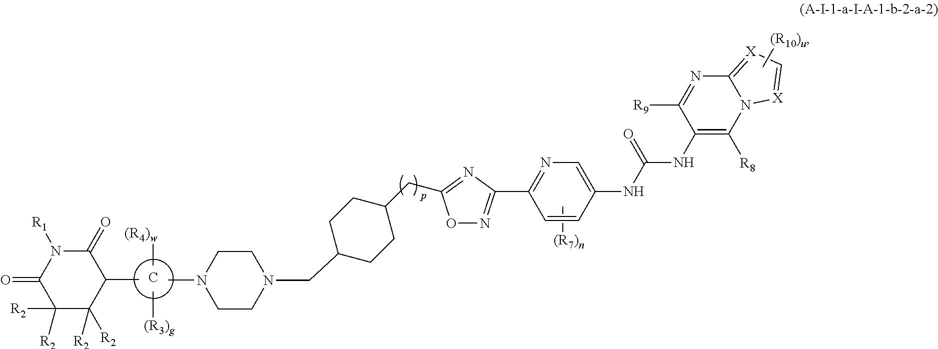

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-2*):

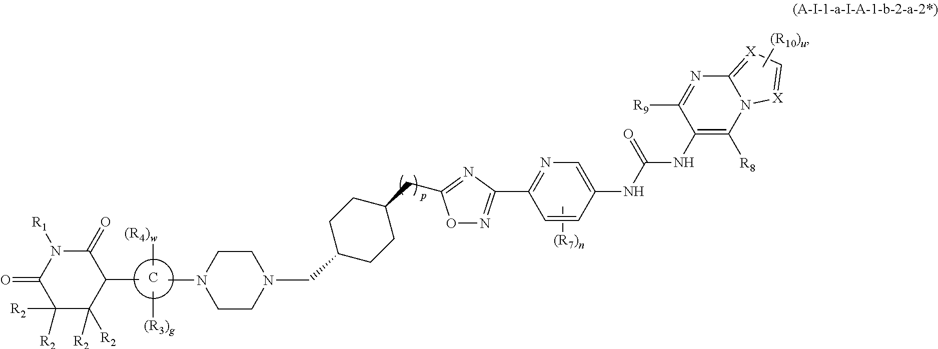

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-2**):

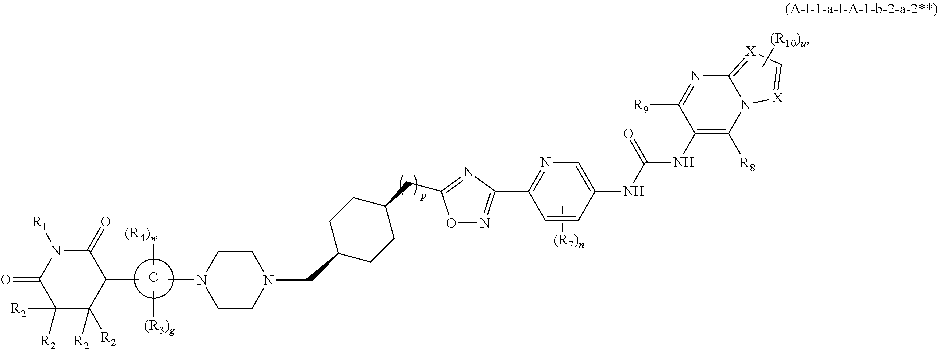

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-2***):

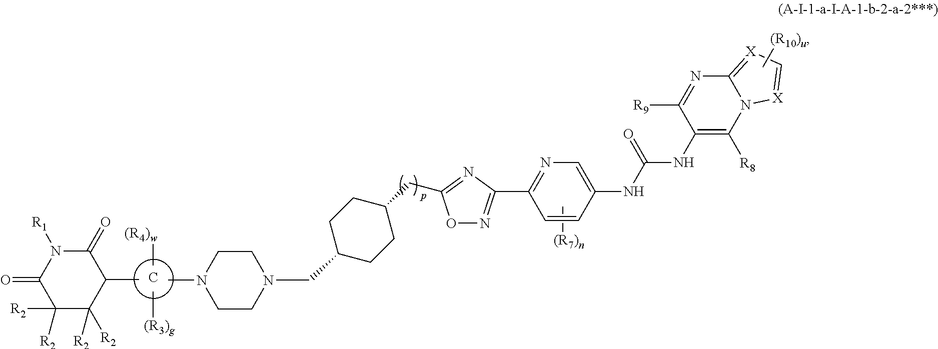

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-b-2-a-2****):

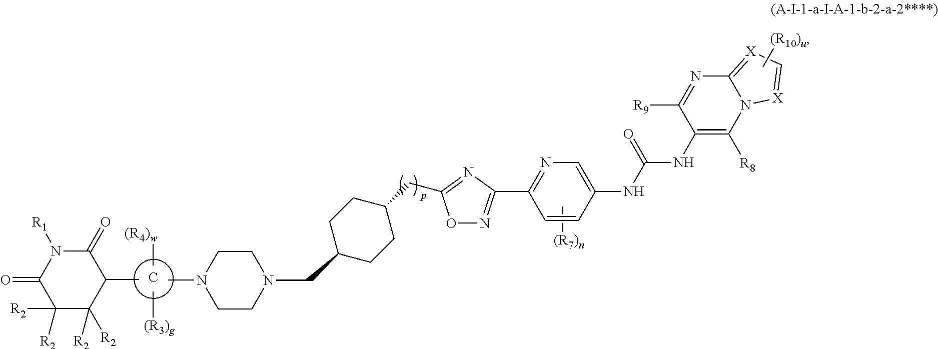

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-1-c):

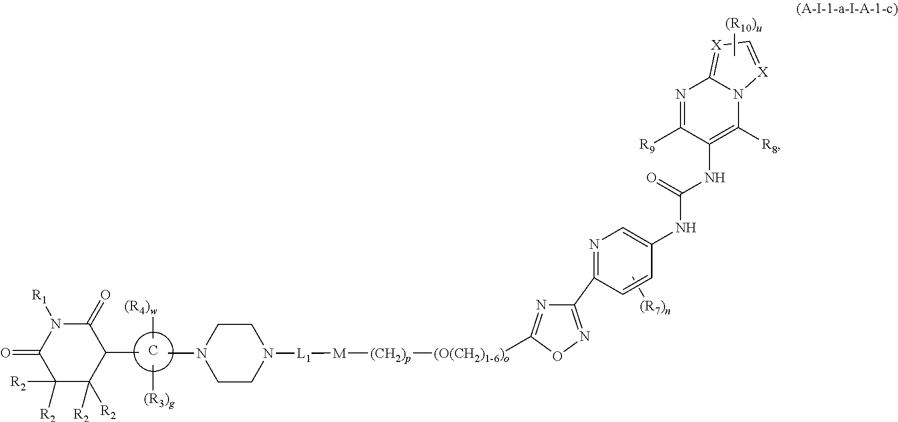

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-2):

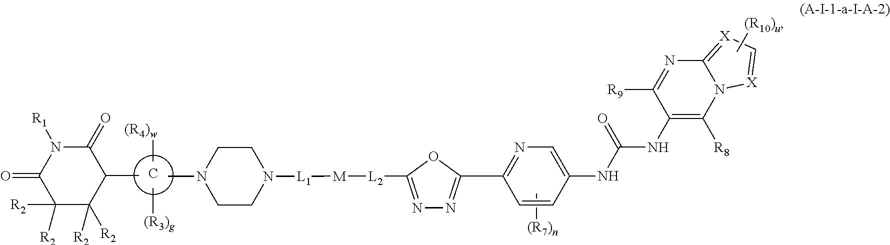

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-2-a):

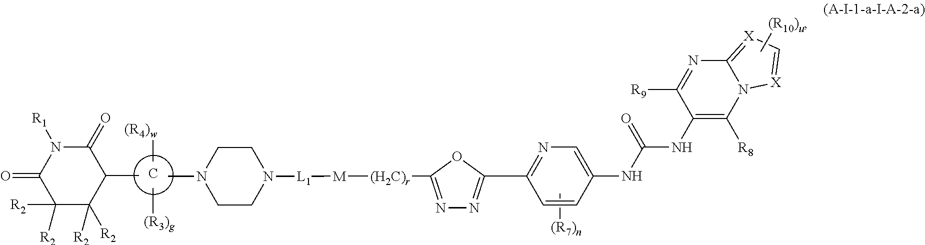

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-3):

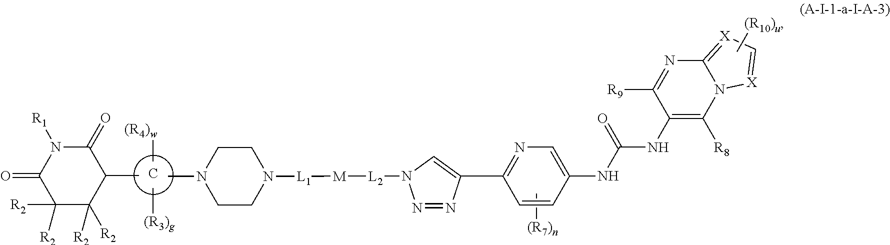

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-3-a):

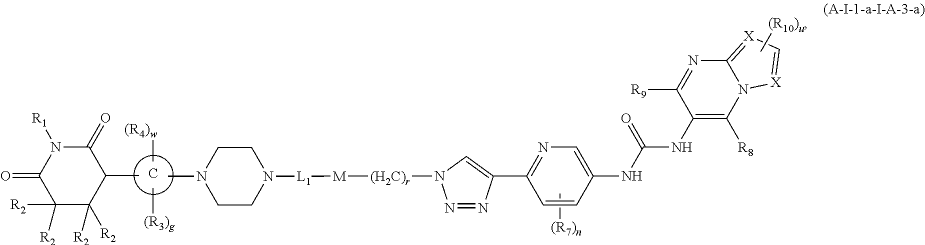

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-3-b):

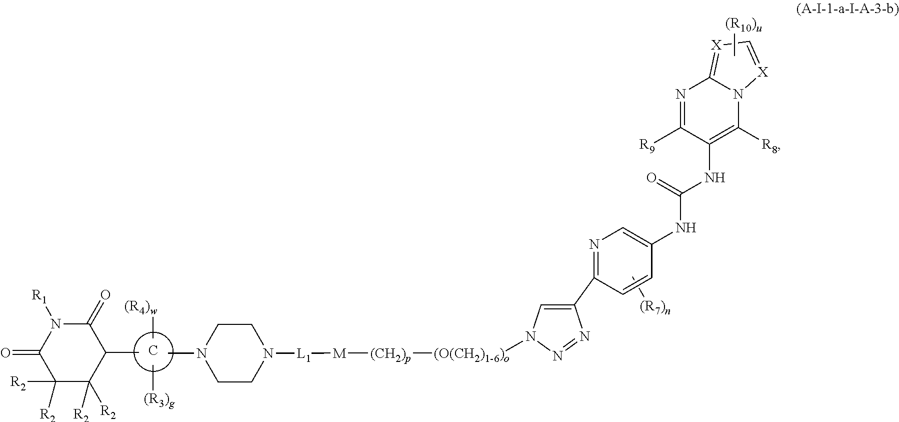

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-4):

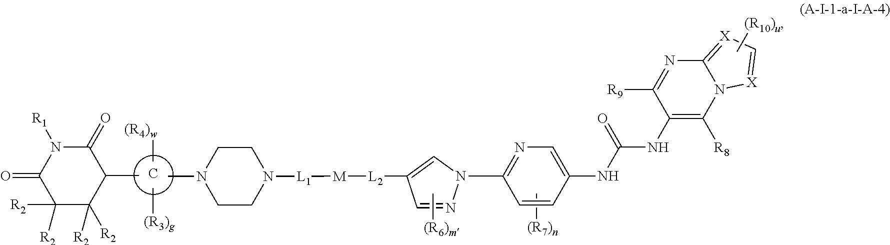

or a pharmaceutically acceptable salt, stereoisomer, solvate, prodrug, isotopic derivative, or tautomer thereof wherein m′ is an integer selected from 0, 1, and 2; and all other variables are as defined herein.

In some embodiments, the compound is of Formula (A-I-1-a-I-A-4-a):

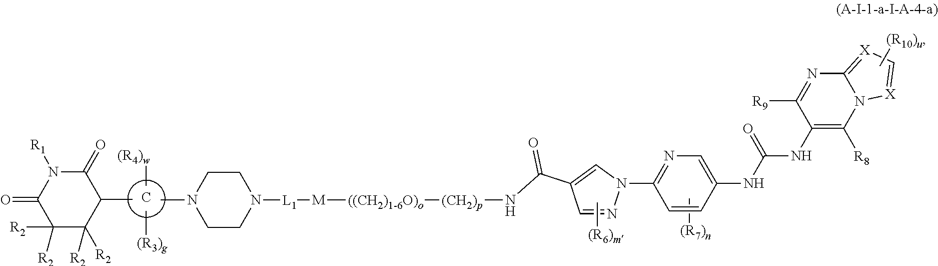

or a pharmaceutically acceptable salt, stereoisomer, solvate, prodrug, isotopic derivative, or tautomer thereof wherein m′ is an integer selected from 0, 1, and 2; and all other variables are as defined herein.

In some embodiments, the compound is of Formula (A-I-1-a-I-A-4-a-1):

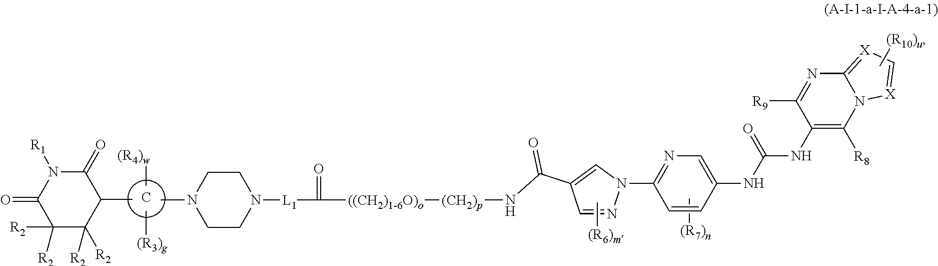

or a pharmaceutically acceptable salt, stereoisomer, solvate, prodrug, isotopic derivative, or tautomer thereof wherein m′ is an integer selected from 0, 1, and 2; and all other variables are as defined herein.

In some embodiments, the compound is of Formula (A-I-1-a-I-A-4-a-1-A):

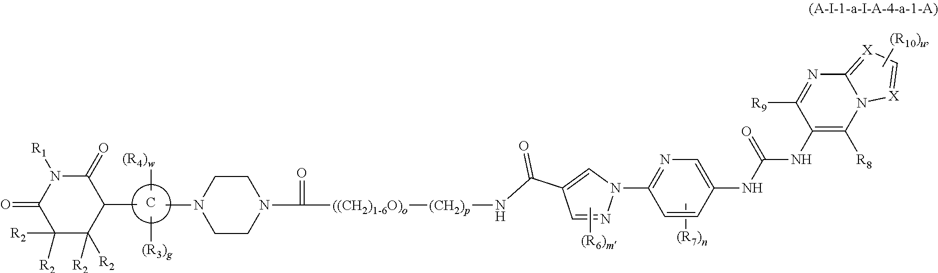

or a pharmaceutically acceptable salt, stereoisomer, solvate, prodrug, isotopic derivative, or tautomer thereof wherein m′ is an integer selected from 0, 1, and 2; and all other variables are as defined herein.

In some embodiments, the compound is of Formula (A-I-1-a-I-A-5):

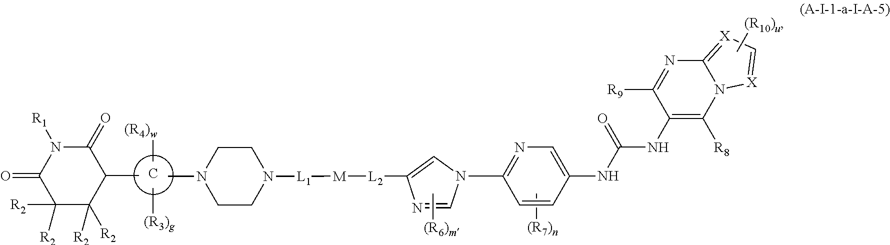

or a pharmaceutically acceptable salt, stereoisomer, solvate, prodrug, isotopic derivative, or tautomer thereof wherein m′ is an integer selected from 0, 1, and 2; and all other variables are as defined herein.

In some embodiments, the compound is of Formula (A-I-1-a-I-A-5-a):

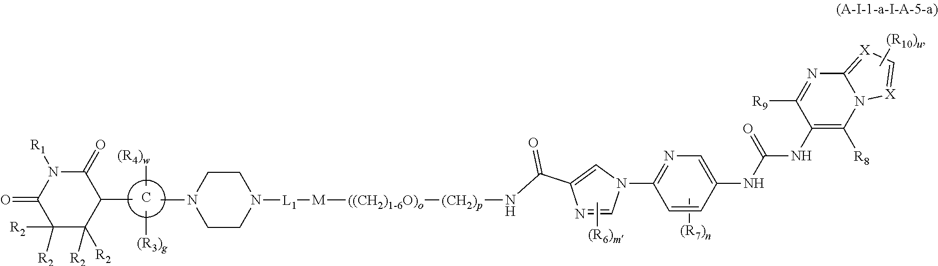

or a pharmaceutically acceptable salt, stereoisomer, solvate, prodrug, isotopic derivative, or tautomer thereof wherein m′ is an integer selected from 0, 1, and 2; and all other variables are as defined herein.

In some embodiments, the compound is of Formula (A-I-1-a-I-A-5-a-1):

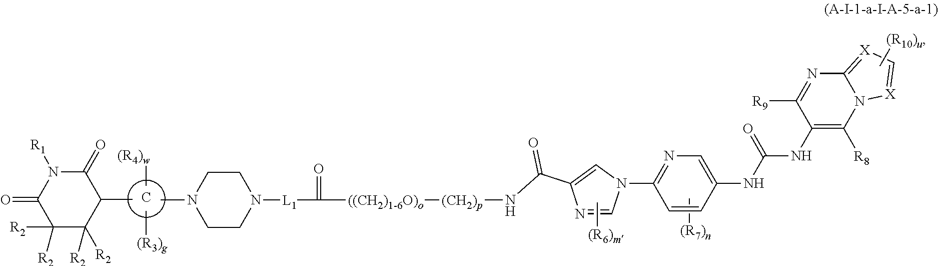

or a pharmaceutically acceptable salt, stereoisomer, solvate, prodrug, isotopic derivative, or tautomer thereof wherein m′ is an integer selected from 0, 1, and 2; and all other variables are as defined herein.

In some embodiments, the compound is of Formula (A-I-1-a-I-A-5-a-1-A):

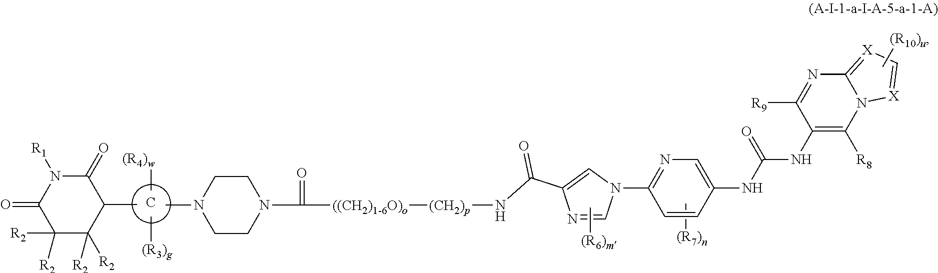

or a pharmaceutically acceptable salt, stereoisomer, solvate, prodrug, isotopic derivative, or tautomer thereof wherein m′ is an integer selected from 0, 1, and 2; and all other variables are as defined herein.

In some embodiments, the compound is of Formula (A-I-1-a-I-A-6):

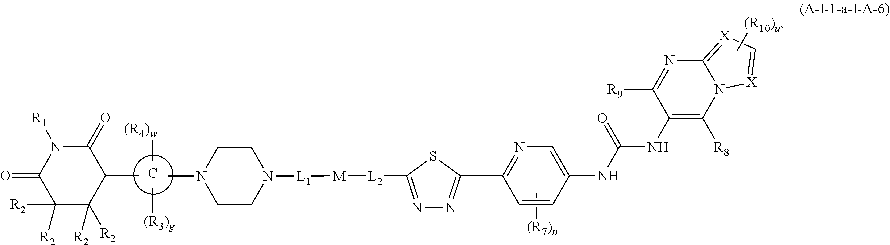

or a pharmaceutically acceptable salt, stereoisomer, solvate, prodrug, isotopic derivative, or tautomer thereof wherein m′ is an integer selected from 0, 1, and 2; and all other variables are as defined herein.

In some embodiments, the compound is of Formula (A-I-1-a-I-A-6-a):

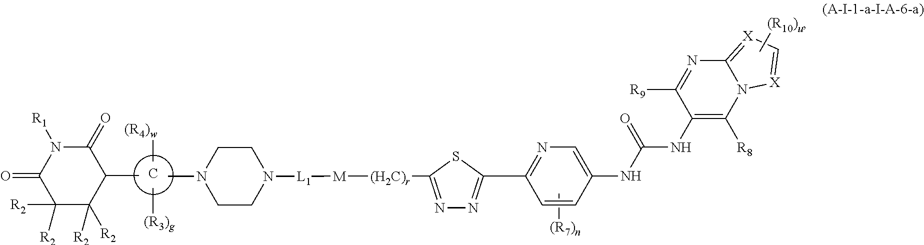

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-6-a-1):

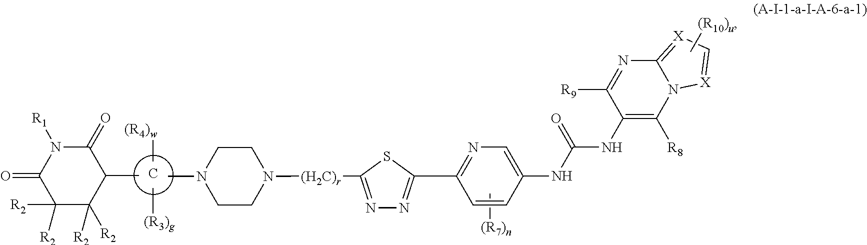

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-6-a-2):

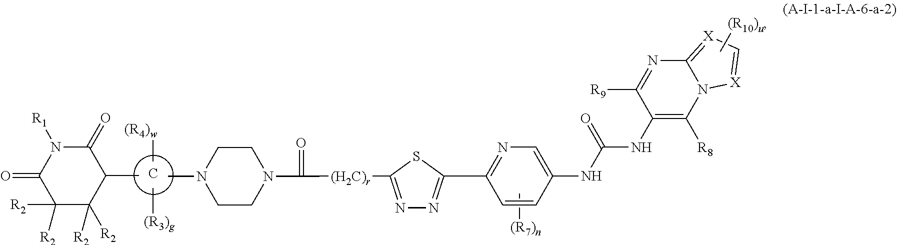

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-1-a-I-A-7):

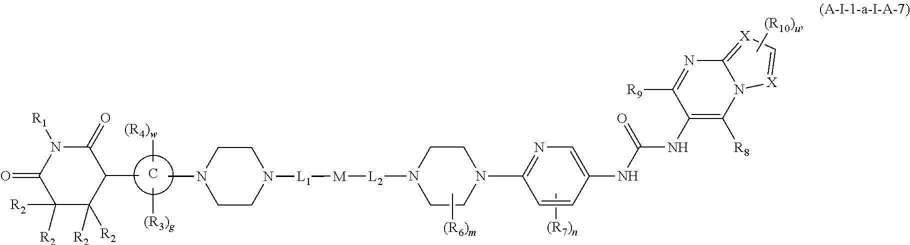

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-7-a):

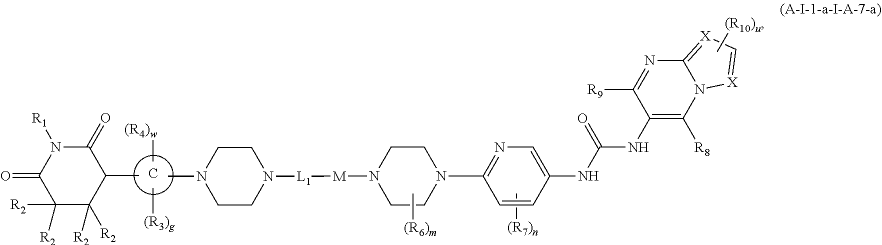

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-7-a-1):

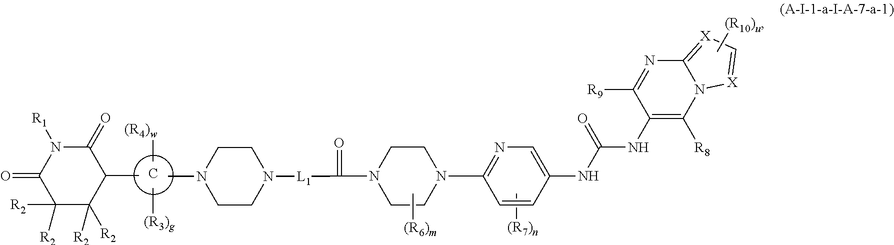

or a pharmaceutically acceptable salt, stereoisomer, solvate, prodrug, isotopic derivative, or tautomer thereof wherein all variables are as defined herein.

In some embodiments, the compound is of Formula (A-I-1-a-I-A-7-a-1-a):

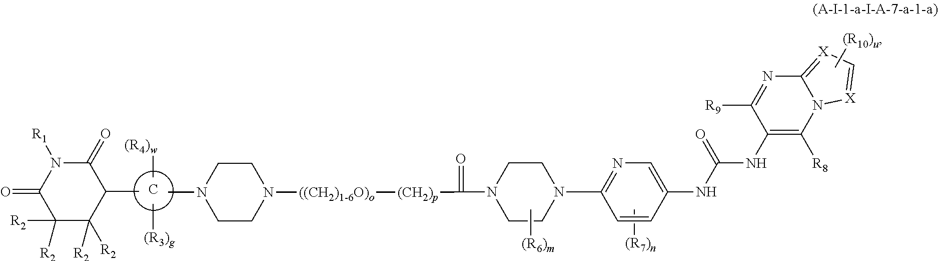

or a pharmaceutically acceptable salt, stereoisomer, solvate, prodrug, isotopic derivative, or tautomer thereof wherein all variables are as defined herein.

In some embodiments, the compound is of Formula (A-I-1-a-II):

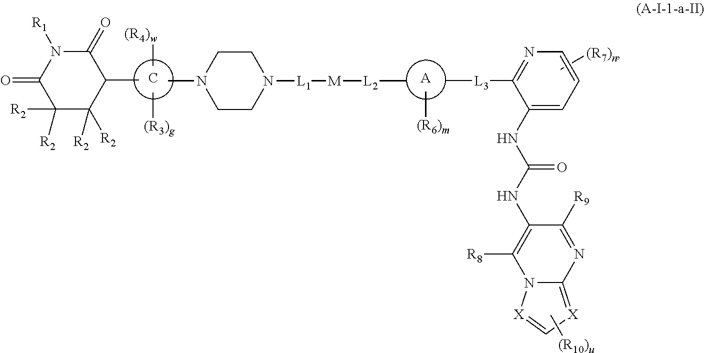

or a pharmaceutically acceptable salt, stereoisomer, solvate, prodrug, isotopic derivative, or tautomer thereof wherein all variables are as defined herein.

In some embodiments, the compound is of Formula (A-I-1-b):

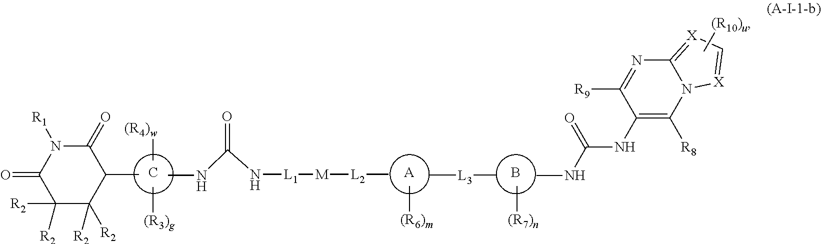

or a pharmaceutically acceptable salt, stereoisomer, solvate, prodrug, isotopic derivative, or tautomer thereof wherein all variables are as defined herein.

In some embodiments, the compound is of Formula (A-I-1-b-I):

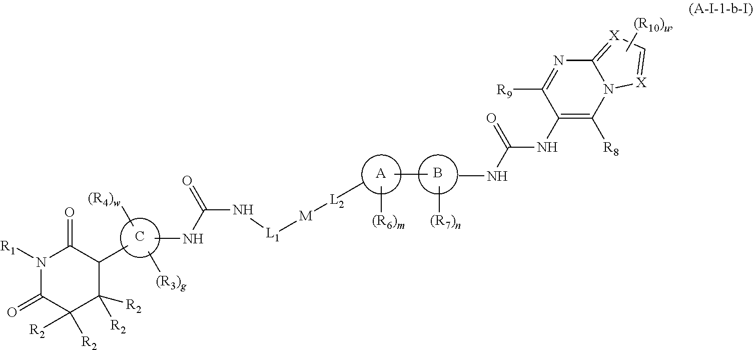

or a pharmaceutically acceptable salt, stereoisomer, solvate, prodrug, isotopic derivative, or tautomer thereof wherein all variables are as defined herein.

In some embodiments, the compound is of Formula (A-I-1-b-I-A):

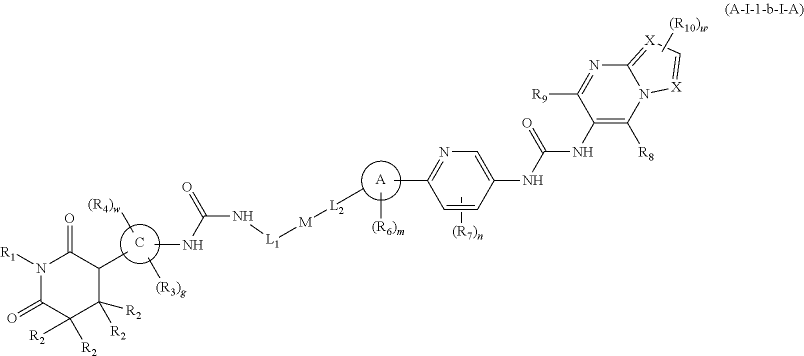

or a pharmaceutically acceptable salt, stereoisomer, solvate, prodrug, isotopic derivative, or tautomer thereof wherein all variables are as defined herein.

In some embodiments, the compound is of Formula (A-I-1-b-I-A-1):

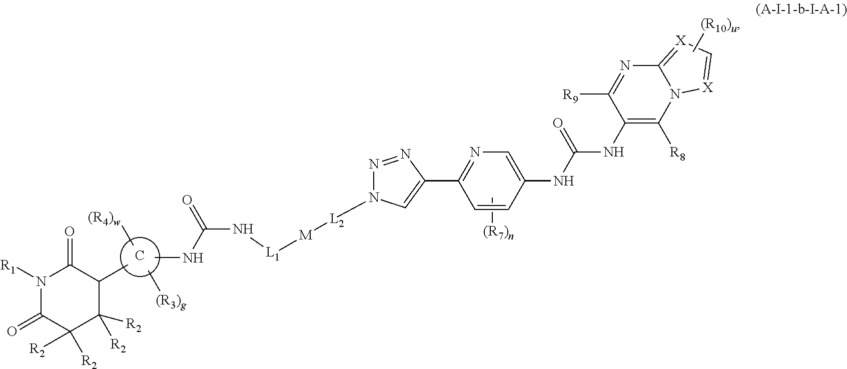

or a pharmaceutically acceptable salt, stereoisomer, solvate, prodrug, isotopic derivative, or tautomer thereof wherein all variables are as defined herein.

In some embodiments, the compound is of Formula (A-I-1-b-I-A-1-a):

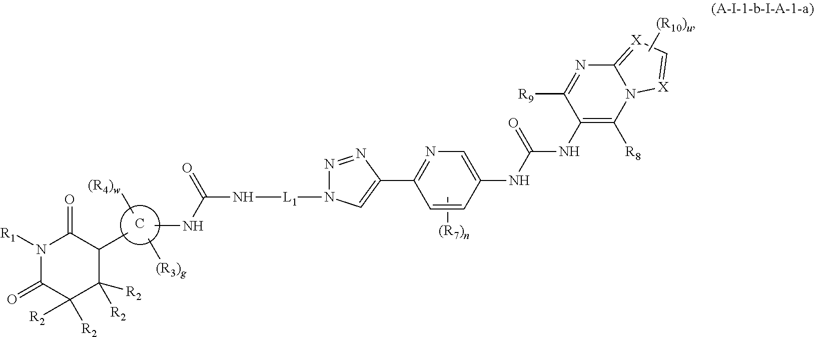

or a pharmaceutically acceptable salt, stereoisomer, solvate, prodrug, isotopic derivative, or tautomer thereof wherein all variables are as defined herein.

In some embodiments, the compound is of Formula (A-I-1-b-I-A-1-a-1):

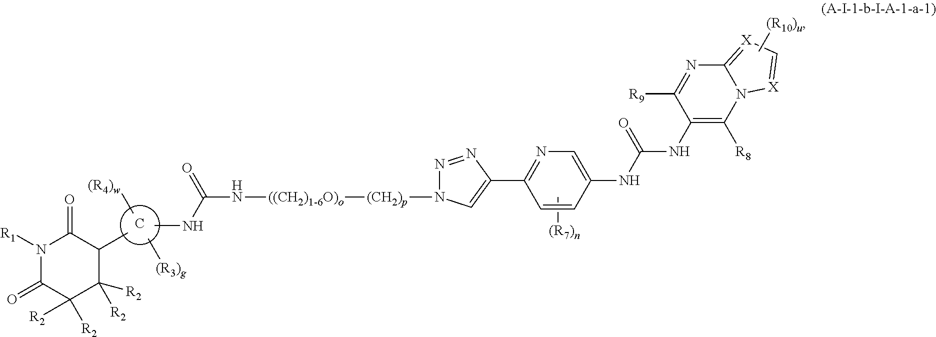

or a pharmaceutically acceptable salt, stereoisomer, solvate, prodrug, isotopic derivative, or tautomer thereof wherein all variables are as defined herein.

In some embodiments, the compound is of Formula (A-I-1-c):

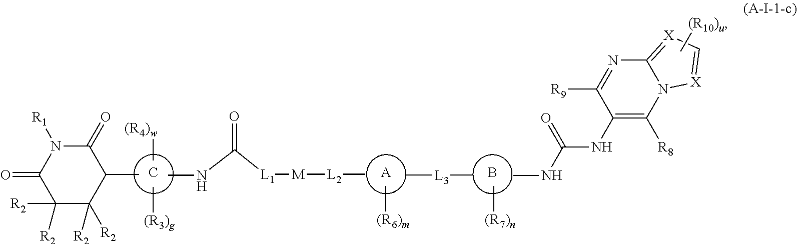

or a pharmaceutically acceptable salt, stereoisomer, solvate, prodrug, isotopic derivative, or tautomer thereof wherein all variables are as defined herein.

In some embodiments, the compound is of Formula (A-I-1-c-I):

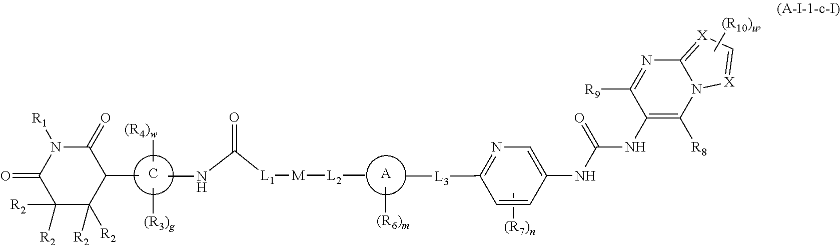

or a pharmaceutically acceptable salt, stereoisomer, solvate, prodrug, isotopic derivative, or tautomer thereof wherein all variables are as defined herein.

In some embodiments, the compound is of Formula (A-I-1-c-I-A):

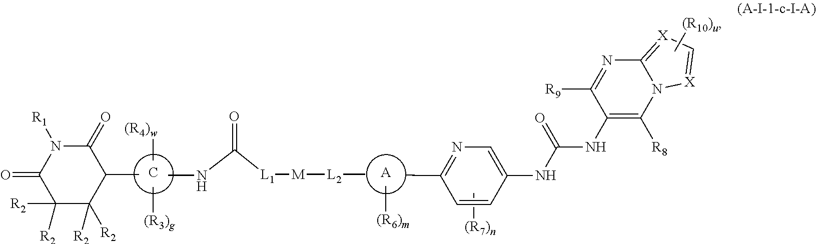

or a pharmaceutically acceptable salt, stereoisomer, solvate, prodrug, isotopic derivative, or tautomer thereof wherein all variables are as defined herein.

In some embodiments, the compound is of Formula (A-I-1-c-I-A-1):

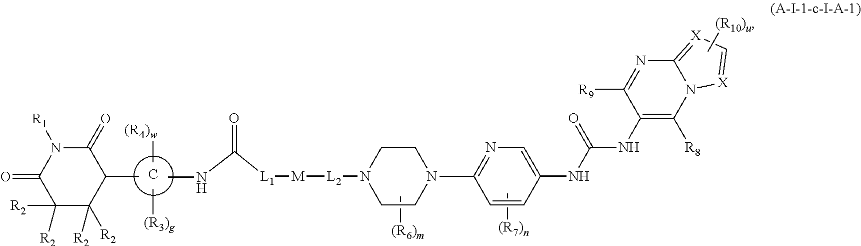

or a pharmaceutically acceptable salt, stereoisomer, solvate, prodrug, isotopic derivative, or tautomer thereof wherein all variables are as defined herein.

In some embodiments, the compound is of Formula (A-I-1-c-I-A-1-a):

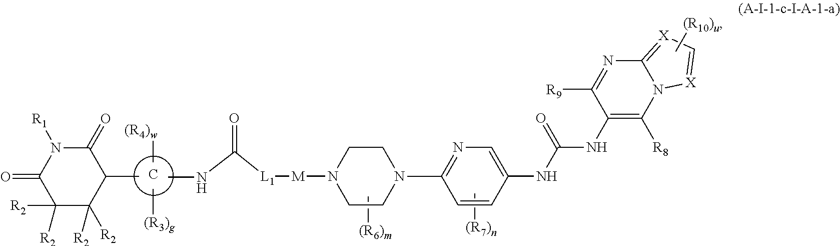

or a pharmaceutically acceptable salt, stereoisomer, solvate, prodrug, isotopic derivative, or tautomer thereof wherein all variables are as defined herein.

In some embodiments, the compound is of Formula (A-I-1-c-I-A-1-a-1):

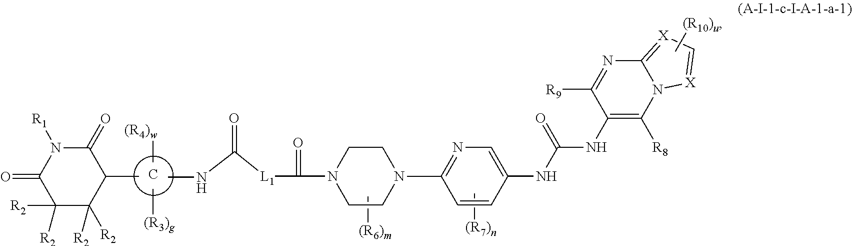

or a pharmaceutically acceptable salt, stereoisomer, solvate, prodrug, isotopic derivative, or tautomer thereof wherein all variables are as defined herein.

In some embodiments, the compound is of Formula (A-I-1-c-I-A-1-a-1-a):

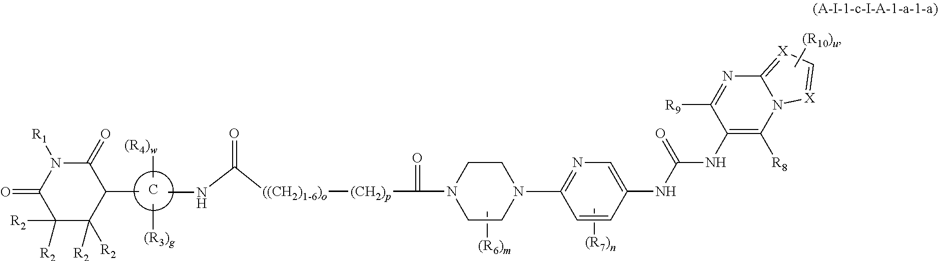

or a pharmaceutically acceptable salt, stereoisomer, solvate, prodrug, isotopic derivative, or tautomer thereof wherein all variables are as defined herein.

In some embodiments, the compound is of Formula (A-I-1-c-I-A-2):

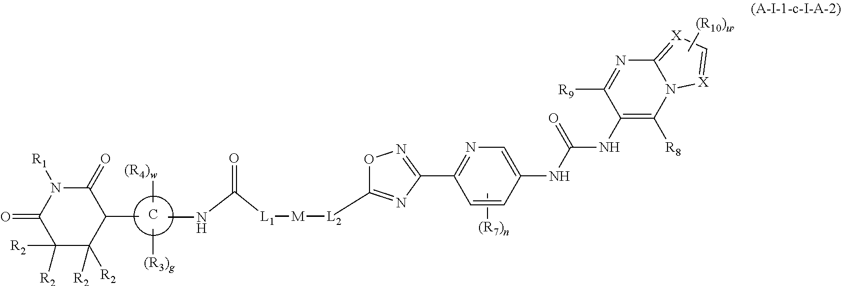

or a pharmaceutically acceptable salt, stereoisomer, solvate, prodrug, isotopic derivative, or tautomer thereof wherein all variables are as defined herein.

In some embodiments, the compound is of Formula (A-I-1-c-I-A-2-a):

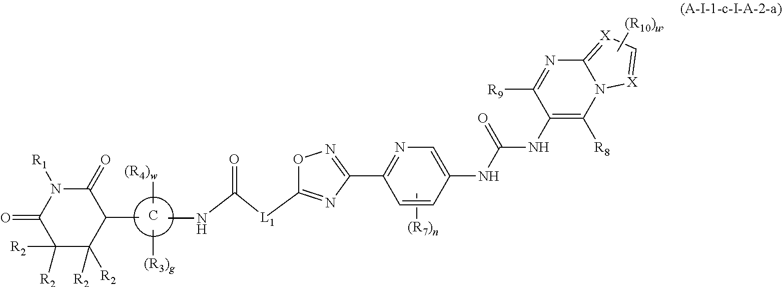

or a pharmaceutically acceptable salt, stereoisomer, solvate, prodrug, isotopic derivative, or tautomer thereof wherein all variables are as defined herein.

In some embodiments, the compound is of Formula (A-I-1-c-I-A-2-a-1):

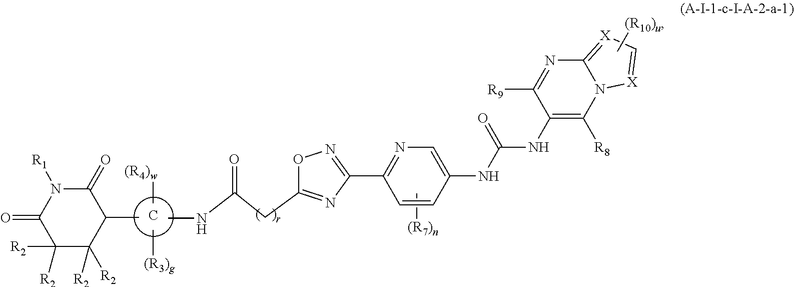

or a pharmaceutically acceptable salt, stereoisomer, solvate, prodrug, isotopic derivative, or tautomer thereof wherein r is an integer selected from 1, 2, 3, 4, 5 and 6; and all other variables are as defined herein.

In some embodiments, the compound is of Formula (A-I-2):

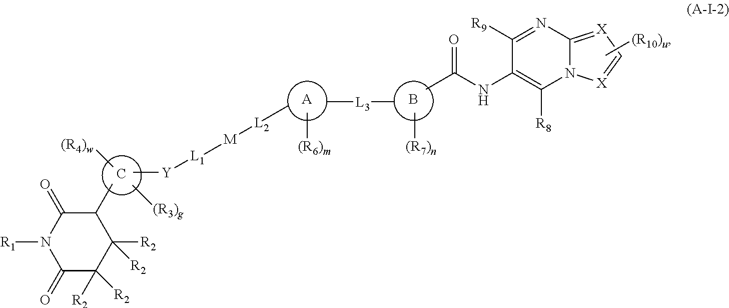

or a pharmaceutically acceptable salt, stereoisomer, solvate, prodrug, isotopic derivative, or tautomer thereof wherein all variables are as defined herein.

In some embodiments, the compound is of Formula (A-II):

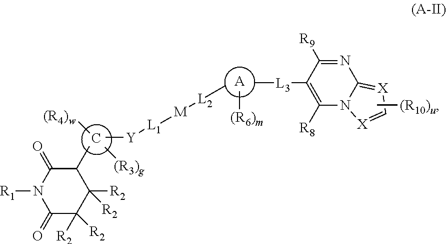

or a pharmaceutically acceptable salt, stereoisomer, solvate, prodrug, isotopic derivative, or tautomer thereof wherein all variables are as defined herein.

In some embodiments, the compound is of Formula (A-II-1):

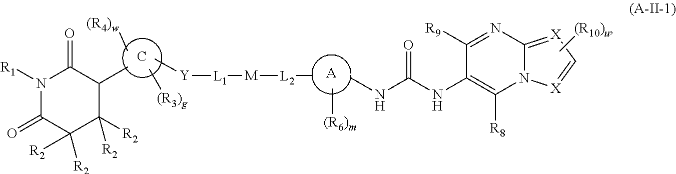

or a pharmaceutically acceptable salt, stereoisomer, solvate, prodrug, isotopic derivative, or tautomer thereof wherein all variables are as defined herein.

In some embodiments, the compound is of Formula (A-II-1-a):

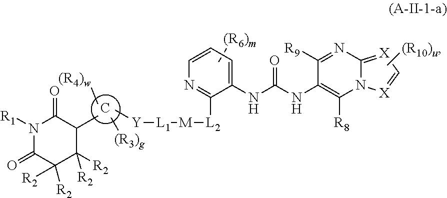

or a pharmaceutically acceptable salt, stereoisomer, solvate, prodrug, isotopic derivative, or tautomer thereof wherein all variables are as defined herein.

In some embodiments, the compound is of Formula (A-II-1-a-I):

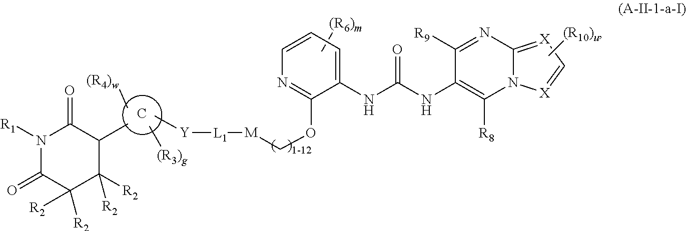

or a pharmaceutically acceptable salt, stereoisomer, solvate, prodrug, isotopic derivative, or tautomer thereof wherein all variables are as defined herein.

In some embodiments, the compound is of Formula (A-II-1-a-I-A):

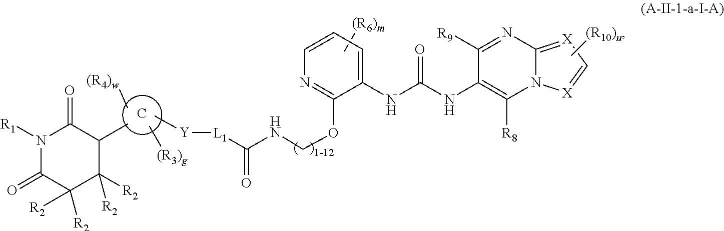

or a pharmaceutically acceptable salt, stereoisomer, solvate, prodrug, isotopic derivative, or tautomer thereof wherein all variables are as defined herein.

In some embodiments, the compound is of Formula (A-II-1-a-I-A-1):

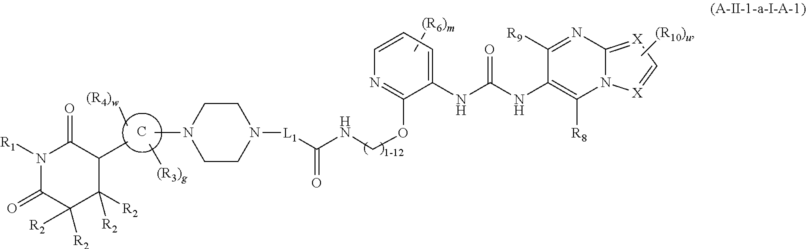

or a pharmaceutically acceptable salt, stereoisomer, solvate, prodrug, isotopic derivative, or tautomer thereof wherein all variables are as defined herein.

In some embodiments, the compound is of Formula (A-II-1-a-I-A-1-a):

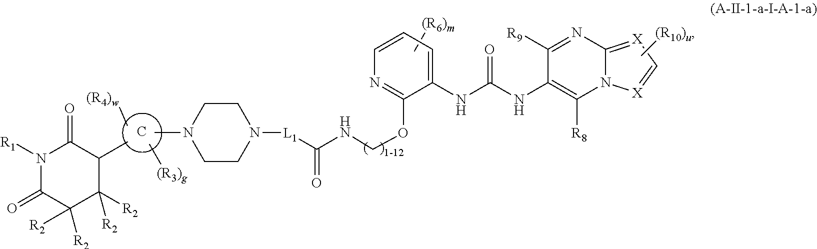

or a pharmaceutically acceptable salt, stereoisomer, solvate, prodrug, isotopic derivative, or tautomer thereof wherein all variables are as defined herein.

In some embodiments, the compound is of Formula (A-II-1-a-I-A-1-a-1):

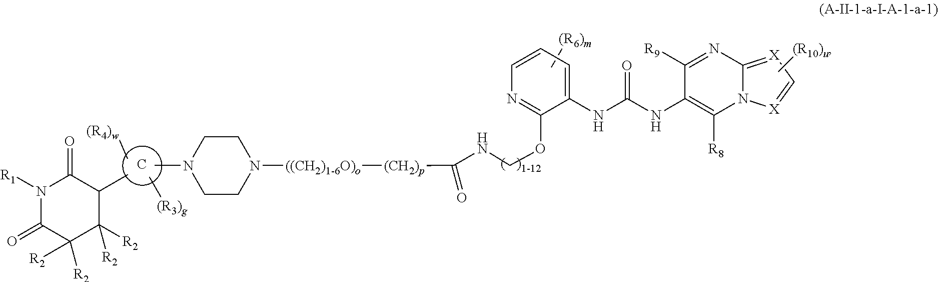

or a pharmaceutically acceptable salt, stereoisomer, solvate, prodrug, isotopic derivative, or tautomer thereof wherein all variables are as defined herein.

In some embodiments, the compound is of Formula (A-II-2):

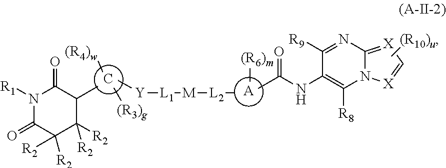

or a pharmaceutically acceptable salt, stereoisomer, solvate, prodrug, isotopic derivative, or tautomer thereof wherein all variables are as defined herein.

In some embodiments, the compound is of Formula (A-II-2-a):

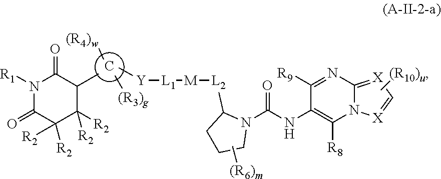

or a pharmaceutically acceptable salt, stereoisomer, solvate, prodrug, isotopic derivative, or tautomer thereof wherein all variables are as defined herein.

In some embodiments, the compound is of Formula (A-II-2-a-I):

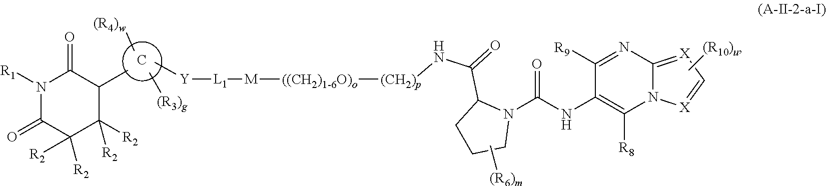

or a pharmaceutically acceptable salt, stereoisomer, solvate, prodrug, isotopic derivative, or tautomer thereof wherein all variables are as defined herein.

In some embodiments, the compound is of Formula (A-II-2-a-I-A):

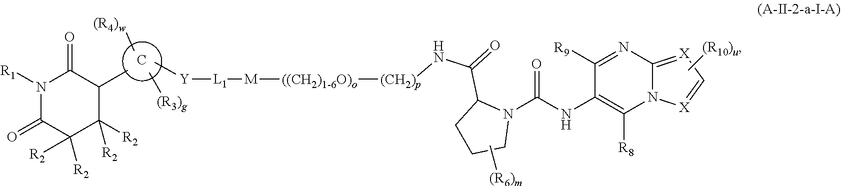

or a pharmaceutically acceptable salt, stereoisomer, solvate, prodrug, isotopic derivative, or tautomer thereof wherein all variables are as defined herein.

In some embodiments, the compound is of Formula (A-II-2-a-I-A):

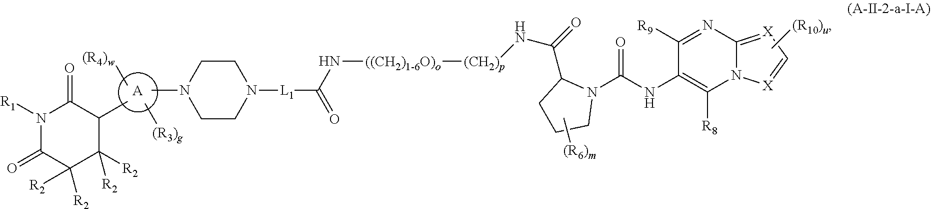

or a pharmaceutically acceptable salt, stereoisomer, solvate, prodrug, isotopic derivative, or tautomer thereof wherein all variables are as defined herein.

In some embodiments, the compound is of Formula (A-II-2-a-I-A-1):

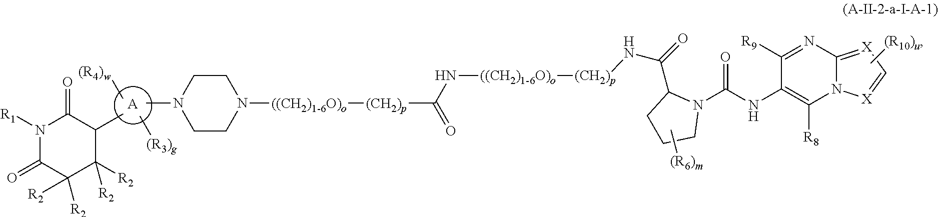

or a pharmaceutically acceptable salt, stereoisomer, solvate, prodrug, isotopic derivative, or tautomer thereof wherein all variables are as defined herein.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

In some embodiments, the compound is selected from the compounds described in Table 1 and pharmaceutically acceptable salts, stereoisomers, solvates, prodrugs, isotopic derivatives, or tautomers thereof.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of the compounds described in Table 1 and pharmaceutically acceptable salts thereof.

In some embodiments, the compound is selected from the compounds described in Table 1.

TABLE 1
Certain examples of the compound of Formula A

| | Structure |
|---|---|
| # | IUPAC Name |
| 1 | 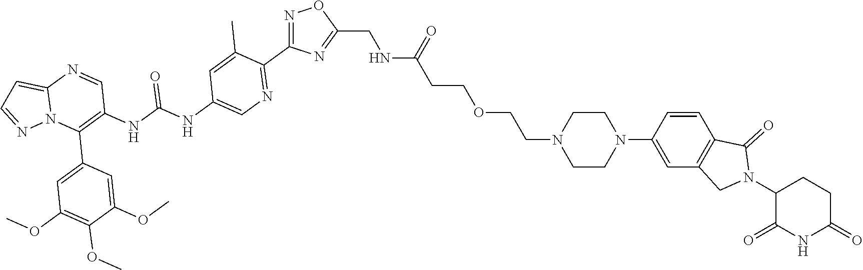 [SMILES: COc1cc(-c2c(NC(=O)Nc3cnc(-c4noc(CNC(=O)CCOCCN5CCN(c6ccc7c(c6)CN(C6CCC(=O)NC6=O)C7=O)CC5)n4)c(C)c3)cnc3ccnn23)cc(OC)c1OC] 3-[2-[4-[2-(2,6-dioxo-3-piperidyl)-1-oxo-isoindolin-5-yl]piperazin-1-yl]ethoxy]-N-[[3- [3-methyl-5-[[7-(3,4,5-trimethoxyphenyl)pyrazolo[1,5-a]pyrimidin-6- yl]carbamoylamino]-2-pyridyl]-1,2,4-oxadiazol-5-yl]methyl]propenamide |
| 2 | 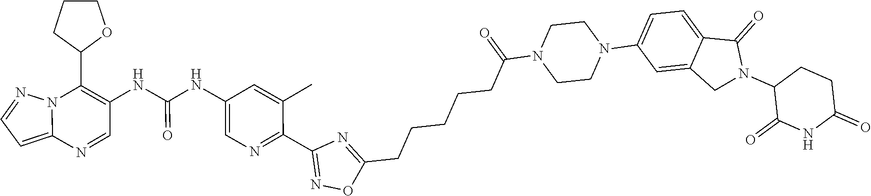 [SMILES: Cc1cc(NC(=O)Nc2cnc3ccnn3c2C2CCCO2)cnc1-c1noc(CCCCCC(=O)N2CCN(c3ccc4c(c3)CN(C3CCC(=O)NC3=O)C4=O)CC2)n1] 1-[6-[5-[6-[4-[2-(2,6-dioxo-3-piperidyl)-1-oxo-isoindolin-5-yl]piperazin-1-yl]-6-oxo- hexyl]-1,2,4-oxadiazol-3-yl]-5-methyl-3-pyridyl]-3-(7-tetrahydrofuran-2- ylpyrazolo[1,5-a]pyrimidin-6-yl)urea |
| 3 | 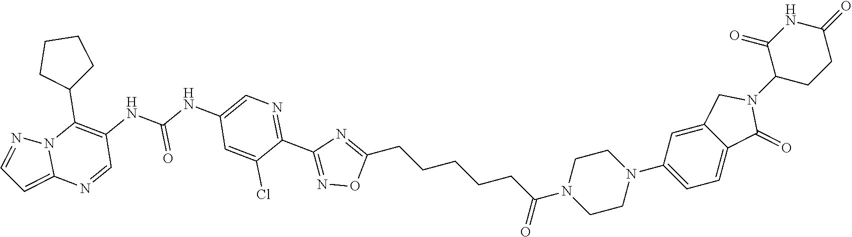 [SMILES: O=C1CCC(N2Cc3cc(N4CCN(C(=O)CCCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-cyclopentylpyrazolo[1,5- a]pyrimidin-6-yl)urea |
| 4 | 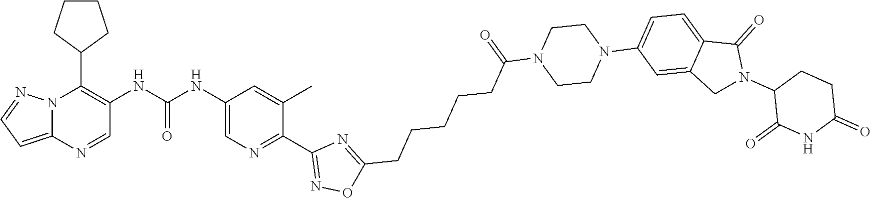 [SMILES: Cc1cc(NC(=O)Nc2cnc3ccnn3c2C2CCCC2)cnc1-c1noc(CCCCCC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 5 | 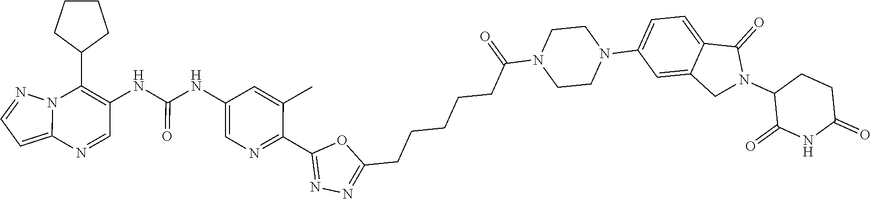 [SMILES: Cc1cc(NC(=O)Nc2cnc3ccnn3c2C2CCCC2)cnc1-c1nnc(CCCCCC(=O)N2CCN(c3ccc4c(c3)CN(C3CCC(=O)NC3=O)C4=O)CC2)o1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,3,4-oxadiazol-2-yl]-5- methyl-3-pyridyl]urea |
| 6 | 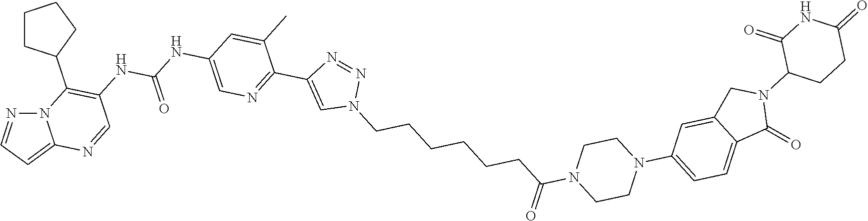 [SMILES: Cc1cc(NC(=O)Nc2cnc3ccnn3c2C2CCCC2)cnc1-c1cn(CCCCCCC(=O)N2CCN(c3ccc4c(c3)CN(C3CCC(=O)NC3=O)C4=O)CC2)nn1] 1-(7-cyclopentylpyrazolo[1,5-a]pyrimidin-6-yl)-3-[6-[1-[7-[4-[2-(2,6-dioxo-3- piperidyl)-1-oxo-isoindolin-5-yl]piperazin-1-yl]-7-oxo-heptyl]triazol-4-yl]-5-methyl-3- pyridyl]urea |
| 7 | 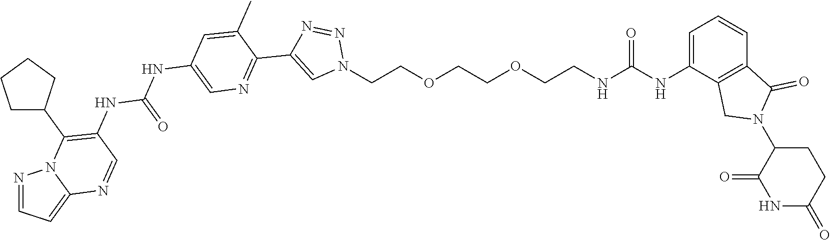 [SMILES: Cc1cc(NC(=O)Nc2cnc3ccnn3c2C2CCCC2)cnc1-c1cn(CCOCCOCCNC(=O)Nc2cccc3c2CN(C2CCC(=O)NC2=O)C3=O)nn1] 1-(7-cyclopentylpyrazolo[1,5-a]pyrimidin-6-yl)-3-[6-[1-[2-[2-[2-[[2-(2,6-dioxo-3- piperidyl)-1-oxo-isoindolin-4-yl]carbamoylamino]ethoxy]ethoxy]ethyl]triazol-4-yl]-5- methyl-3-pyridyl]urea |
| 8 | 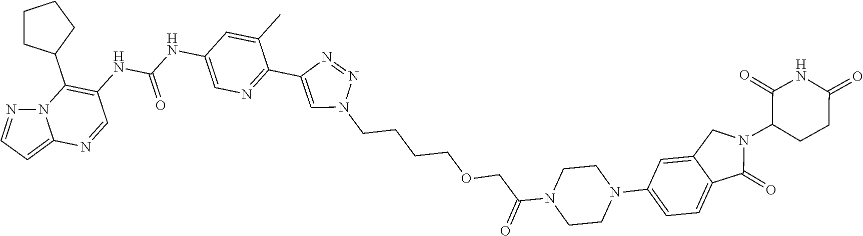 [SMILES: Cc1cc(NC(=O)Nc2cnc3ccnn3c2C2CCCC2)cnc1-c1cn(CCCCOCC(=O)N2CCN(c3ccc4c(c3)CN(C3CCC(=O)NC3=O)C4=O)CC2)nn1] 1-(7-cyclopentylpyrazolo[1,5-a]pyrimidin-6-yl)-3-[6-[1-[4-[2-[4-[2-(2,6-dioxo-3- piperidyl)-1-oxo-isoindolin-5-yl]piperazin-1-yl]-2-oxo-ethoxy]butyl]triazol-4-yl]-5- methyl-3-pyridyl]urea |
| 9 | 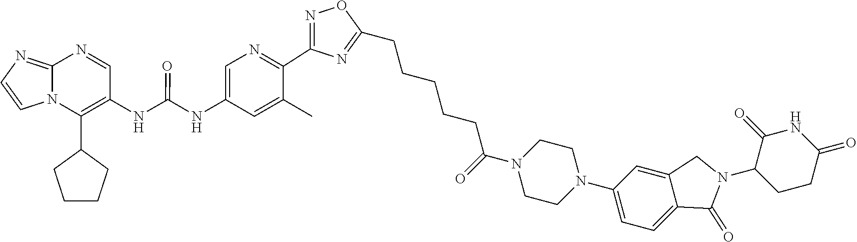 [SMILES: Cc1cc(NC(=O)Nc2cnc3nccn3c2C2CCCC2)cnc1-c1noc(CCCCCC(=O)N2CCN(c3ccc4c(c3)CN(C3CCC(=O)NC3=O)C4=O)CC2)n1] 1-(5-cyclopentylimidazo[1,2-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 10 | 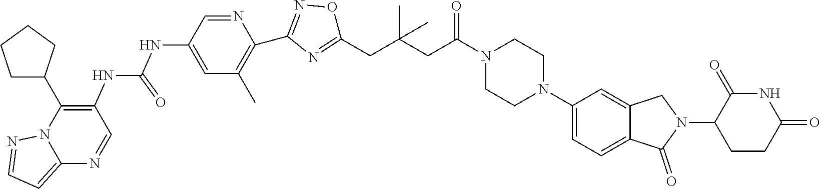 [SMILES: Cc1cc(NC(=O)Nc2cnc3ccnn3c2C2CCCC2)cnc1-c1noc(CC(C)(C)CC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-2,2-dimethyl-4-oxo-butyl]-1,2,4- oxadiazol-3-yl]-5-methyl-3-pyridyl]urea |
| 11 | 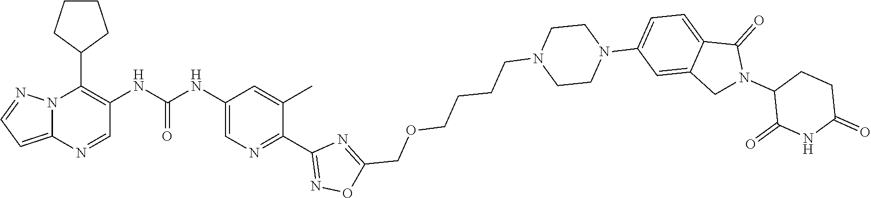 [SMILES: Cc1cc(NC(=O)Nc2cnc3ccnn3c2C2CCCC2)cnc1-c1noc(COCCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butoxymethyl]-1,2,4-oxadiazol-3-yl]- 5-methyl-3-pyridyl]urea |
| 12 | 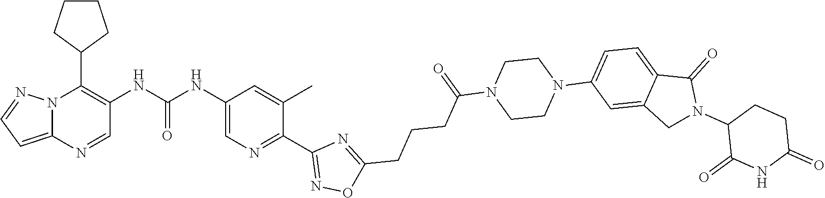 [SMILES: Cc1cc(NC(=O)Nc2cnc3ccnn3c2C2CCCC2)cnc1-c1noc(CCCC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- methyl-3-pyridyl]urea |
| 13 | 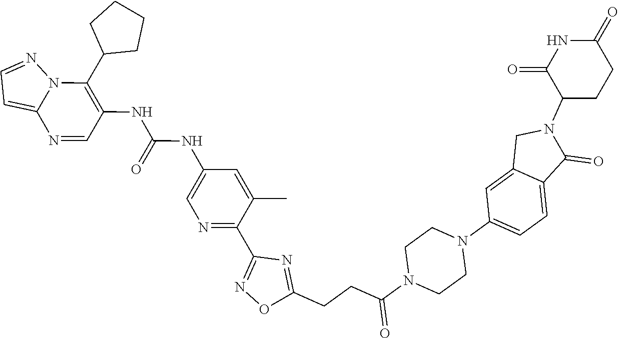 [SMILES: Cc1cc(NC(=O)Nc2cnc3ccnn3c2C2CCCC2)cnc1-c1noc(CCC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[3-[4-[2-(2,6-dioxo-3- piperidyl)-1-oxo-isoindolin-5-yl]piperazin-1-yl]-3-oxo-propyl]-1,2,4-oxadiazol-3-yl]- 5-methyl-3-pyridyl]urea |
| 14 | 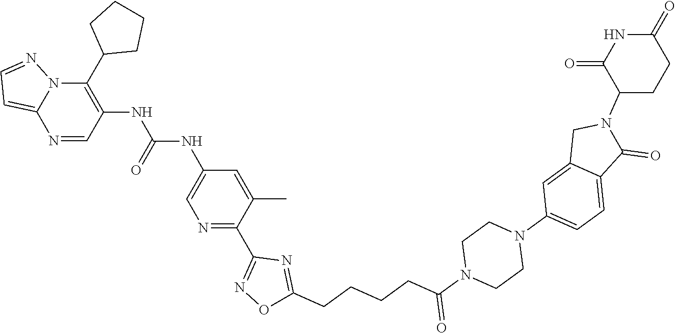 [SMILES: Cc1cc(NC(=O)Nc2cnc3ccnn3c2C2CCCC2)cnc1-c1noc(CCCCC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- methyl-3-pyridyl]urea |
| 15 | 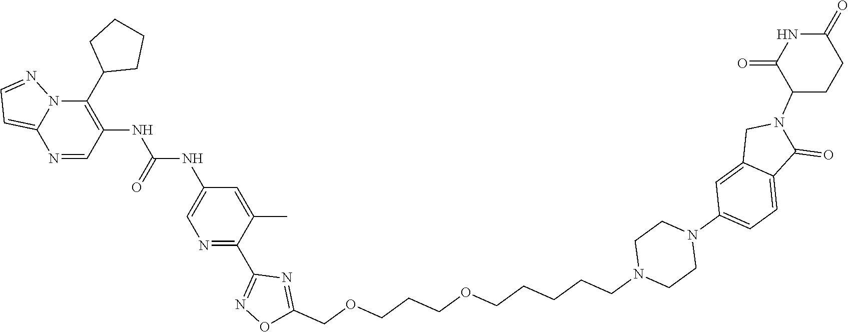 [SMILES: Cc1cc(NC(=O)Nc2cnc3ccnn3c2C2CCCC2)cnc1-c1noc(COCCCOCCCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[3-[5-[4-[2-(2,6-dioxo-3- piperidyl)-1-oxo-isoindolin-5-yl]piperazin-1-yl]pentoxy]propoxymethyl]-1,2,4- oxadiazol-3-yl]-5-methyl-3-pyridyl]urea |
| 16 | 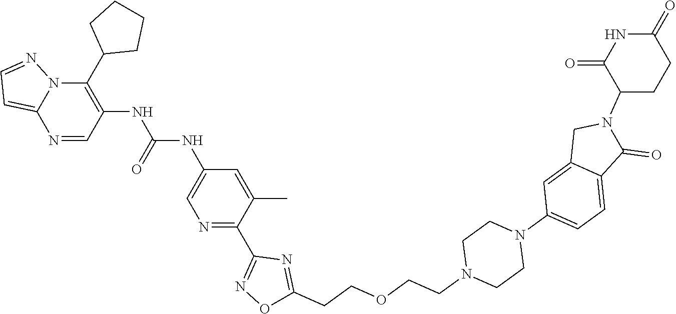 [SMILES: Cc1cc(NC(=O)Nc2cnc3ccnn3c2C2CCCC2)cnc1-c1noc(CCOCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[2-[2-[4-[2-(2,6-dioxo-3- piperidyl)-1-oxo-isoindolin-5-yl]piperazin-1-yl]ethoxy]ethyl]-1,2,4-oxadiazol-3-yl]-5- methyl-3-pyridyl]urea |
| 17 | 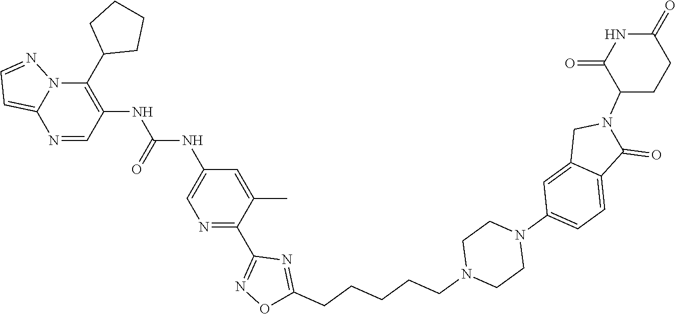 [SMILES: Cc1cc(NC(=O)Nc2cnc3ccnn3c2C2CCCC2)cnc1-c1noc(CCCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- methyl-3-pyridyl]urea |
| 18 | 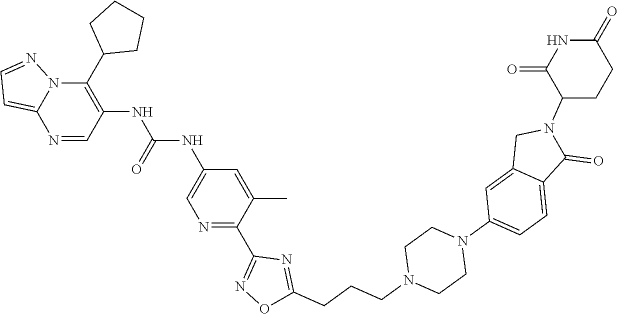 [SMILES: Cc1cc(NC(=O)Nc2cnc3ccnn3c2C2CCCC2)cnc1-c1noc(CCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[3-[4-[2-(2,6-dioxo-3- piperidyl)-1-oxo-isoindolin-5-yl]piperazin-1-yl]propyl]-1,2,4-oxadiazol-3-yl]-5- methyl-3-pyridyl]urea |
| 19 | 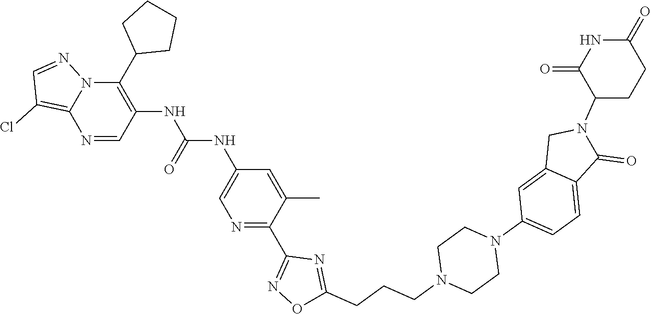 [SMILES: Cc1cc(NC(=O)Nc2cnc3c(Cl)cnn3c2C2CCCC2)cnc1-c1noc(CCCN2CCN(c3ccc4c(c3)CN(C3CCC(=O)NC3=O)C4=O)CC2)n1] 1-(3-chloro-7-cyclopentyl-pyrazolo[1,5-a]pyrimidin-6-yl)-3-[6-[5-[3-[4-[2-(2,6-dioxo- 3-piperidyl)-1-oxo-isoindolin-5-yl]piperazin-1-yl]propyl]-1,2,4-oxadiazol-3-yl]-5- methyl-3-pyridyl]urea |
| 20 | 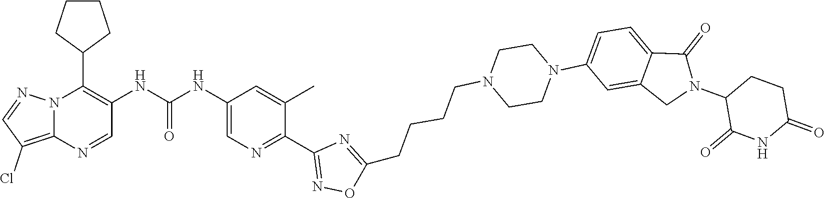 [SMILES: Cc1cc(NC(=O)Nc2cnc3c(Cl)cnn3c2C2CCCC2)cnc1-c1noc(CCCCN2CCN(c3ccc4c(c3)CN(C3CCC(=O)NC3=O)C4=O)CC2)n1] 1-(3-chloro-7-cyclopentyl-pyrazolo[1,5-a]pyrimidin-6-yl)-3-[6-[5-[4-[4-[2-(2,6-dioxo- 3-piperidyl)-1-oxo-isoindolin-5-yl]piperazin-1-yl]butyl]-1,2,4-oxadiazol-3-yl]-5- methyl-3-pyridyl]urea |
| 21 | 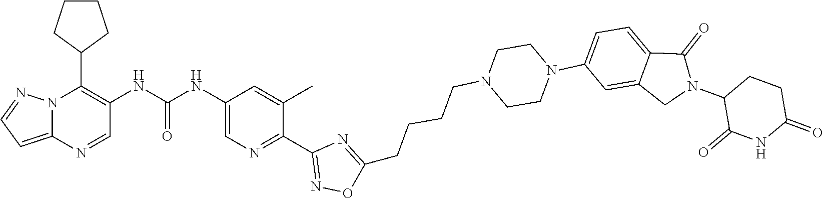 [SMILES: Cc1cc(NC(=O)Nc2cnc3ccnn3c2C2CCCC2)cnc1-c1noc(CCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butyl]-1,2,4-oxadiazol-3-yl]-5-methyl- 3-pyridyl]urea |
| 22 | 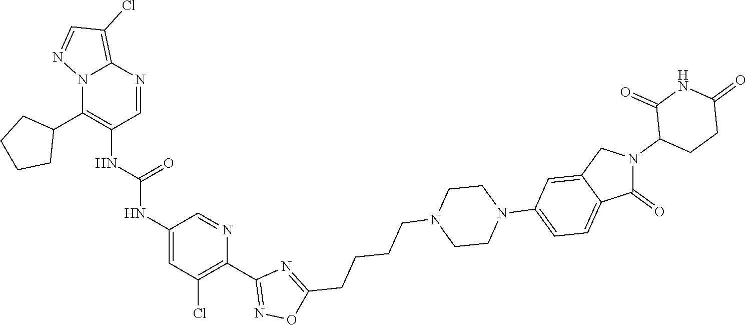 [SMILES: O=C1CCC(N2Cc3cc(N4CCN(CCCCc5nc(-c6ncc(NC(=O)Nc7cnc8c(Cl)cnn8c7C7CCCC7)cc6Cl)no5)CC4)ccc3C2=O)C(=O)N1] 1-(3-chloro-7-cyclopentyl-pyrazolo[1,5-a]pyrimidin-6-yl)-3-[5-chloro-6-[5-[4-[4-[2- (2,6-dioxo-3-piperidyl)-1-oxo-isoindolin-5-yl]piperazin-1-yl]butyl]-1,2,4-oxadiazol-3- yl]-3-pyridyl]urea |
| 23 | 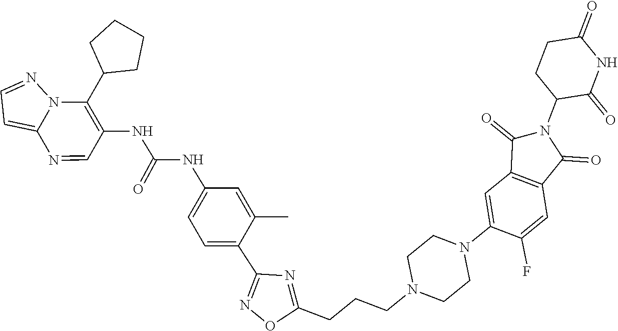 [SMILES: Cc1cc(NC(=O)Nc2cnc3ccnn3c2C2CCCC2)ccc1-c1noc(CCCN2CCN(c3cc4c(cc3F)C(=O)N(C3CCC(=O)NC3=O)C4=O)CC2)n1] 1-(7-cyclopentylpyrazolo[1,5-a]pyrimidin-6-yl)-3-[6-[5-[3-[4-[2-(2,6-dioxo-3- piperidyl)-6-fluoro-1,3-dioxo-isoindolin-5-yl]piperazin-1-yl]propyl]-1,2,4-oxadiazol-3- yl]-5-methyl-3-pyridyl]urea |
| 24 | 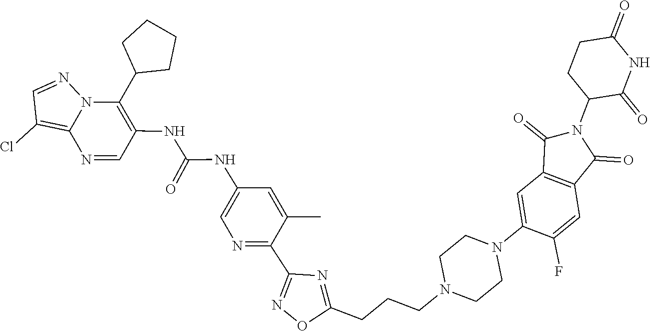 [SMILES: Cc1cc(NC(=O)Nc2cnc3c(Cl)cnn3c2C2CCCC2)cnc1-c1noc(CCCN2CCN(c3cc4c(cc3F)C(=O)N(C3CCC(=O)NC3=O)C4=O)CC2)n1] 1-(3-chloro-7-cyclopentyl-pyrazolo[1,5-a]pyrimidin-6-yl)-3-[6-[5-[3-[4-[2-(2,6-dioxo- 3-piperidyl)-6-fluoro-1,3-dioxo-isoindolin-5-yl]piperazin-1-yl]propyl]-1,2,4-oxadiazol- 3-yl]-5-methyl-3-pyridyl]urea |
| 25 | 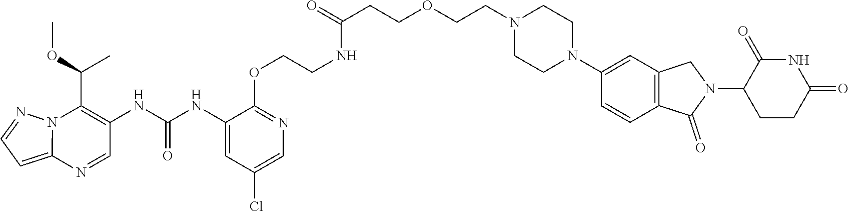 [SMILES: CO[C@@H](C)c1c(NC(=O)Nc2cc(Cl)cnc2OCCNC(=O)CCOCCN2CCN(c3ccc4c(c3)CN(C3CCC(=O)NC3=O)C4=O)CC2)cnc2ccnn12] N-[2-[[5-chloro-3-[[7-[(1S)-1-methoxyethyl]pyrazolo[1,5-a]pyrimidin-6- yl]carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 26 | 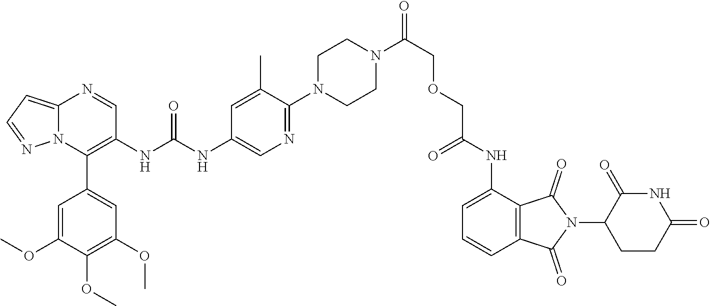 [SMILES: COc1cc(-c2c(NC(=O)Nc3cnc(N4CCN(C(=O)COCC(=O)Nc5cccc6c5C(=O)N(C5CCC(=O)NC5=O)C6=O)CC4)c(C)c3)cnc3ccnn23)cc(OC)c1OC] N-[2-(2,6-dioxo-3-piperidyl)-1,3-dioxo-isoindolin-4-yl]-2-[2-[4-[3-methyl-5-[[7- (3,4,5-trimethoxyphenyl)pyrazolo[1,5-a]pyrimidin-6-yl]carbamoylamino]-2- pyridyl]piperazin-1-yl]-2-oxo-ethoxy]acetamide |
| 27 | 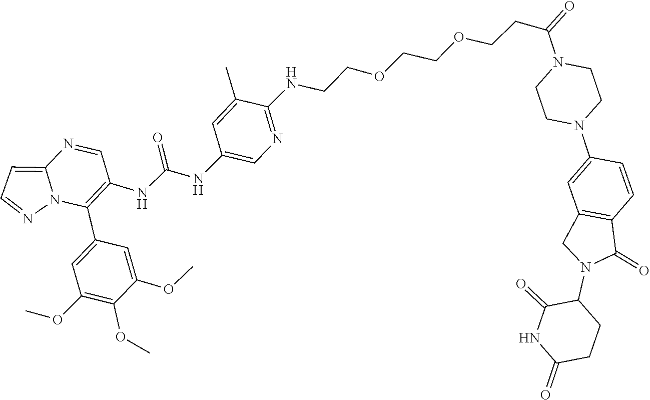 [SMILES: COc1cc(-c2c(NC(=O)Nc3cnc(NCCOCCOCCC(=O)N4CCN(c5ccc6c(c5)CN(C5CCC(=O)NC5=O)C6=O)CC4)c(C)c3)cnc3ccnn23)cc(OC)c1OC] 1-[6-[2-[2-[3-[4-[2-(2,6-dioxo-3-piperidyl)-1-oxo-isoindolin-5-yl]piperazin-1-yl]-3- oxo-propoxy]ethoxy]ethylamino]-5-methyl-3-pyridyl]-3-[7-(3,4,5- trimethoxyphenyl)pyrazolo[1,5-a]pyrimidin-6-yl]urea |
| 28 | 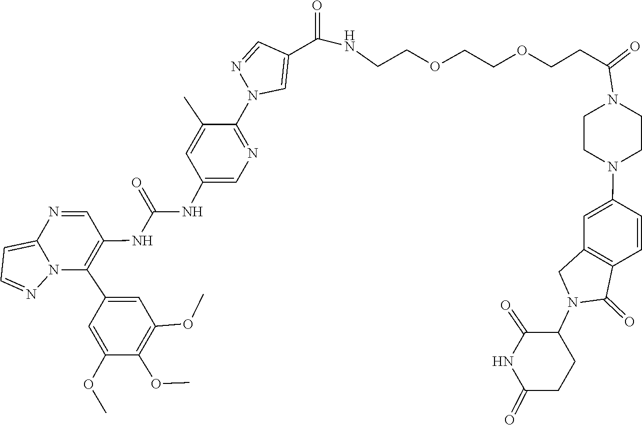 [SMILES: COc1cc(-c2c(NC(=O)Nc3cnc(-n4cc(C(=O)NCCOCCOCCC(=O)N5CCN(c6ccc7c(c6)CN(C6CCC(=O)NC6=O)C7=O)CC5)cn4)c(C)c3)cnc3ccnn23)cc(OC)c1OC] N-[2-[2-[3-[4-[2-(2,6-dioxo-3-piperidyl)-1-oxo-isoindolin-5-yl]piperazin-1-yl]-3-oxo- propoxy]ethoxy]ethyl]-1-[3-methyl-5-[[7-(3,4,5-trimethoxyphenyl)pyrazolo[1,5- a]pyrimidin-6-yl]carbamoylamino]-2-pyridyl]pyrazole-4-carboxamide |
| 29 | 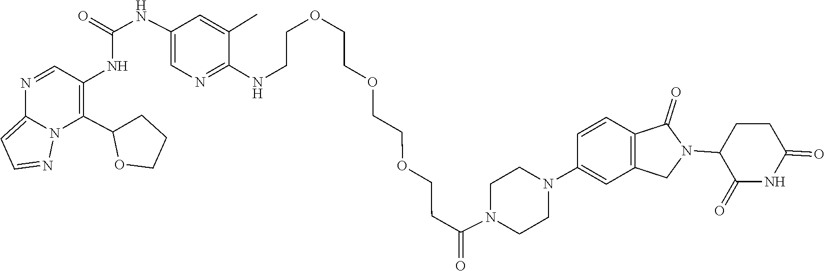 [SMILES: Cc1cc(NC(=O)Nc2cnc3ccnn3c2C2CCCO2)cnc1NCCOCCOCCOCCC(=O)N1CCN(c2ccc3c(c2)CN(C2CCC(=O)NC2=O)C3=O)CC1] 1-[6-[2-[2-[2-[3-[4-[2-(2,6-dioxo-3-piperidyl)-1-oxo-isoindolin-5-yl]piperazin-1-yl]-3- oxo-propoxy]ethoxy]ethoxy]ethylamino]-5-methyl-3-pyridyl]-3-(7-tetrahydrofuran-2- ylpyrazolo[1,5-a]pyrimidin-6-yl)urea |
| 30 | 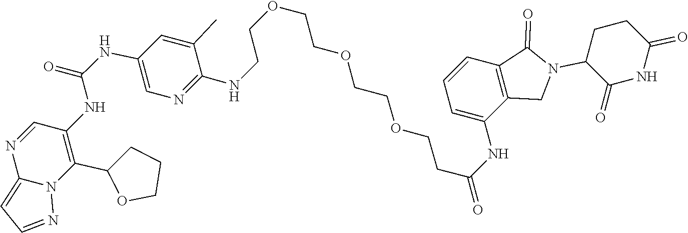 [SMILES: Cc1cc(NC(=O)Nc2cnc3ccnn3c2C2CCCO2)cnc1NCCOCCOCCOCCC(=O)Nc1cccc2c1CN(C1CCC(=O)NC1=O)C2=O] N-[2-(2,6-dioxo-3-piperidyl)-1-oxo-isoindolin-4-yl]-3-[2-[2-[2-[3-methyl-5-[(7- tetrahydrofuran-2-ylpyrazolo[1,5-a]pyrimidin-6-yl)carbamoylamino]-2- pyridyl]amino]ethoxy]ethoxy]ethoxy]propanamide |
| 31 | 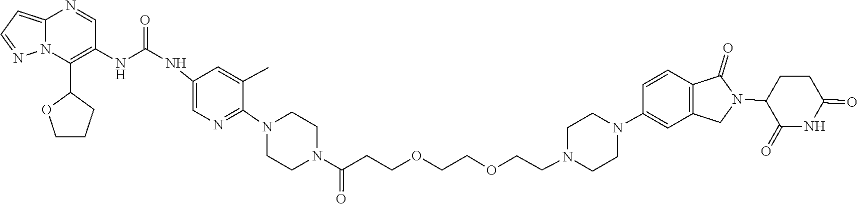 [SMILES: Cc1cc(NC(=O)Nc2cnc3ccnn3c2C2CCCO2)cnc1N1CCN(C(=O)CCOCCOCCN2CCN(c3ccc4c(c3)CN(C3CCC(=O)NC3=O)C4=O)CC2)CC1] 1-[6-[4-[3-[2-[2-[4-[2-(2,6-dioxo-3-piperidyl)-1-oxo-isoindolin-5-yl]piperazin-1- yl]ethoxy]ethoxy]propanoyl]piperazin-1-yl]-5-methyl-3-pyridyl]-3-(7-tetrahydrofuran- 2-ylpyrazolo[1,5-a]pyrimidin-6-yl)urea |
| 32 | 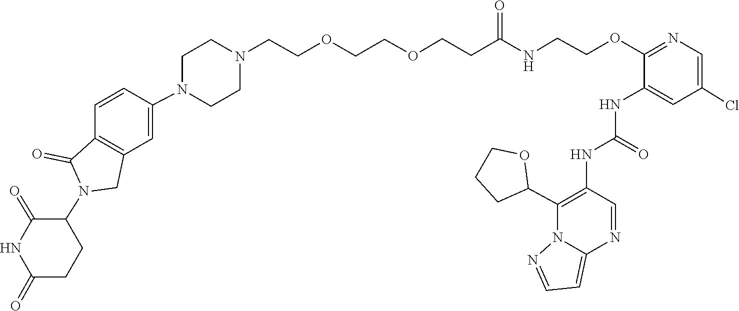 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCO1] N-[2-[[5-chloro-3-[(7-tetrahydrofuran-2-ylpyrazolo[1,5-a]pyrimidin-6- yl)carbamoylamino]-2-pyridyl]oxy]ethyl]-3-[2-[2-[4-[2-(2,6-dioxo-3-piperidyl)-1-oxo- isoindolin-5-yl]piperazin-1-yl]ethoxy]ethoxy]propanamide |
| 33 | 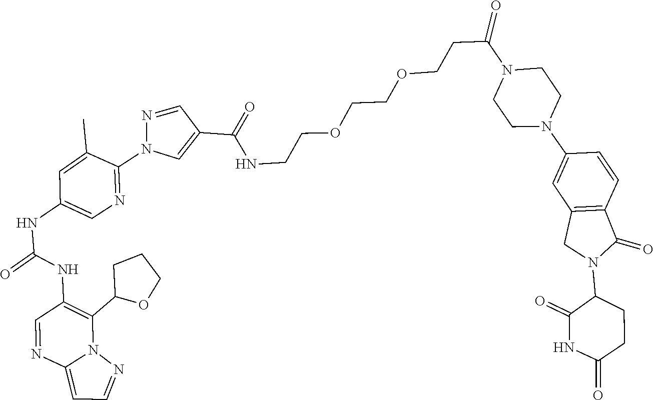 [SMILES: Cc1cc(NC(=O)Nc2cnc3ccnn3c2C2CCCO2)cnc1-n1cc(C(=O)NCCOCCOCCC(=O)N2CCN(c3ccc4c(c3)CN(C3CCC(=O)NC3=O)C4=O)CC2)cn1] N-[2-[2-[3-[4-[2-(2,6-dioxo-3-piperidyl)-1-oxo-isoindolin-5-yl]piperazin-1-yl]-3-oxo- propoxy]ethoxy]ethyl]-1-[3-methyl-5-[(7-tetrahydrofuran-2-ylpyrazolo[1,5- a]pyrimidin-6-yl)carbamoylamino]-2-pyridyl]pyrazole-4-carboxamide |
| 34 | 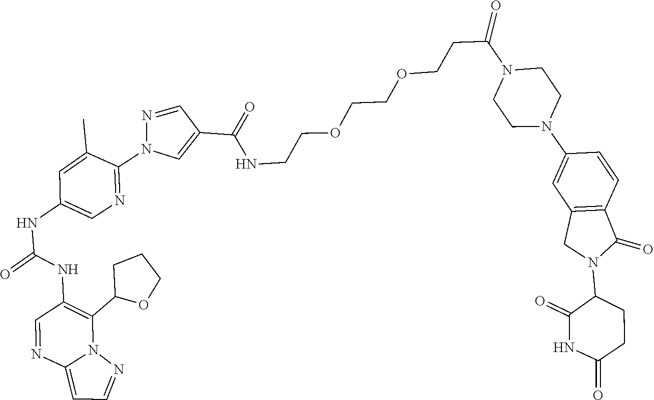 [SMILES: Cc1cc(NC(=O)Nc2cnc3ccnn3c2C2CCCO2)cnc1-n1cc(C(=O)NCCOCCOCCC(=O)N2CCN(c3ccc4c(c3)CN(C3CCC(=O)NC3=O)C4=O)CC2)cn1] N-[2-[2-[3-[4-[2-(2,6-dioxo-3-piperidyl)-1-oxo-isoindolin-5-yl]piperazin-1-yl]-3-oxo- propoxy]ethoxy]ethyl]-1-[3-methyl-5-[(7-tetrahydrofuran-2-ylpyrazolo[1,5- a]pyrimidin-6-yl)carbamoylamino]-2-pyridyl]imidazole-4-carboxamide |
| 35 | 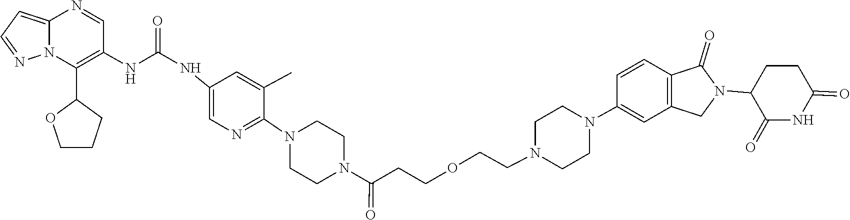 [SMILES: Cc1cc(NC(=O)Nc2cnc3ccnn3c2C2CCCO2)cnc1N1CCN(C(=O)CCOCCN2CCN(c3ccc4c(c3)CN(C3CCC(=O)NC3=O)C4=O)CC2)CC1] 1-[6-[4-[3-[2-[4-[2-(2,6-dioxo-3-piperidyl)-1-oxo-isoindolin-5-yl]piperazin-1- yl]ethoxy]propanoyl]piperazin-1-yl]-5-methyl-3-pyridyl]-3-(7-tetrahydrofuran-2- ylpyrazolo[1,5-a]pyrimidin-6-yl)urea |
| 36 | 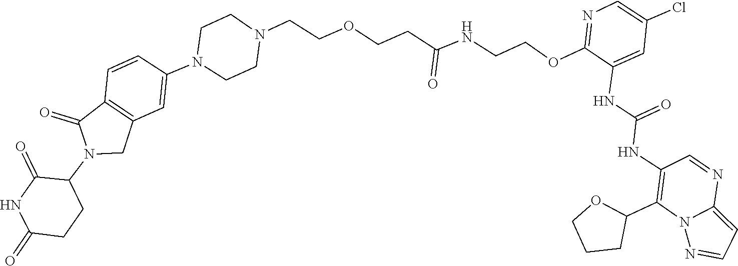 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCO1] N-[2-[[5-chloro-3-[(7-tetrahydrofuran-2-ylpyrazolo[1,5-a]pyrimidin-6- yl)carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 37 | 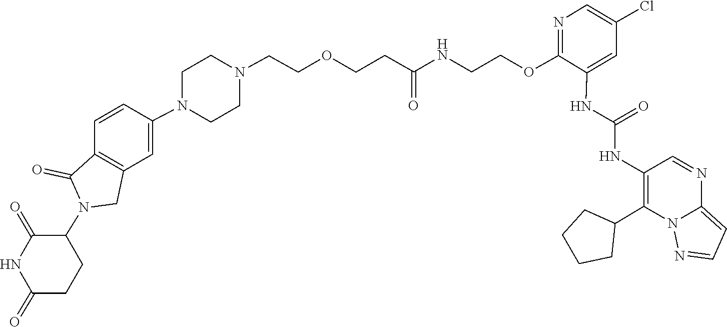 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCC1] N-[2-[[5-chloro-3-[(7-cyclopentylpyrazolo[1,5-a]pyrimidin-6-yl)carbamoylamino]-2- pyridyl]oxy]ethyl]-3-[2-[4-[2-(2,6-dioxo-3-piperidyl)-1-oxo-isoindolin-5-yl]piperazin- 1-yl]ethoxy]propanamide |
| 38 | 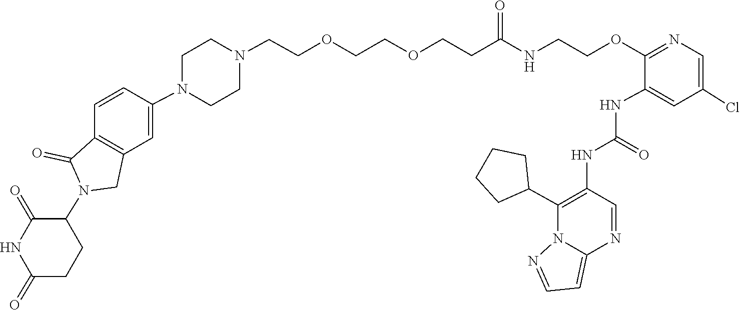 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCC1] N-[2-[[5-chloro-3-[(7-cyclopentylpyrazolo[1,5-a]pyrimidin-6-yl)carbamoylamino]-2- pyridyl]oxy]ethyl]-3-[2-[2-[4-[2-(2,6-dioxo-3-piperidyl)-1-oxo-isoindolin-5- yl]piperazin-1-yl]ethoxy]ethoxy]propanamide |
| 39 | 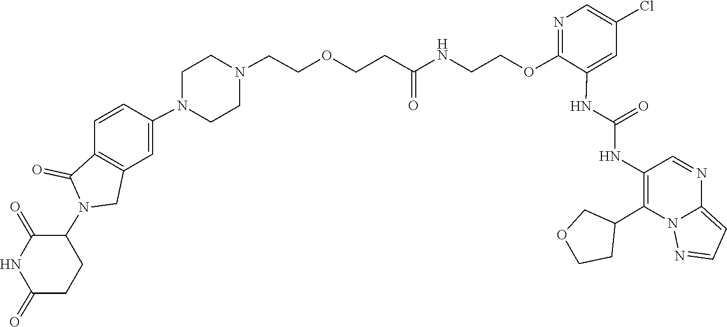 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCOC1] N-[2-[[5-chloro-3-[(7-tetrahydrofuran-3-ylpyrazolo[1,5-a]pyrimidin-6- yl)carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 40 | 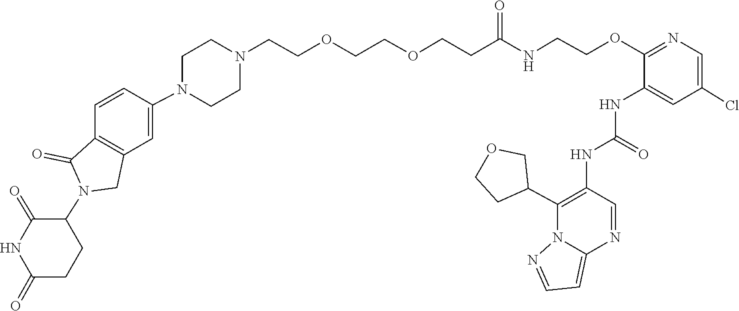 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCOC1] N-[2-[[5-chloro-3-[(7-tetrahydrofuran-3-ylpyrazolo[1,5-a]pyrimidin-6- yl)carbamoylamino]-2-pyridyl]oxy]ethyl]-3-[2-[2-[4-[2-(2,6-dioxo-3-piperidyl)-1-oxo- isoindolin-5-yl]piperazin-1-yl]ethoxy]ethoxy]propanamide |
| 41 | 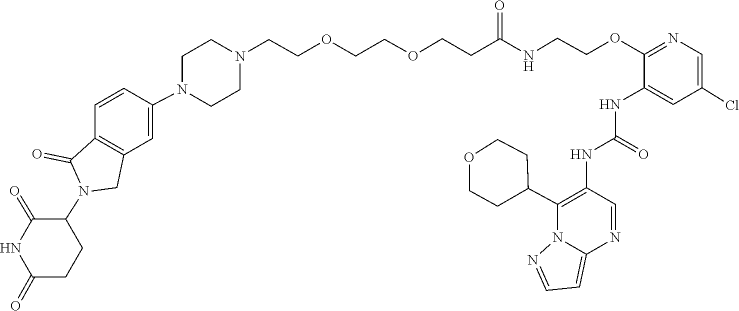 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCOCC1] N-[2-[[5-chloro-3-[(7-tetrahydropyran-4-ylpyrazolo[1,5-a]pyrimidin-6- yl)carbamoylamino]-2-pyridyl]oxy]ethyl]-3-[2-[2-[4-[2-(2,6-dioxo-3-piperidyl)-1-oxo- isoindolin-5-yl]piperazin-1-yl]ethoxy]ethoxy]propanamide |
| 42 | 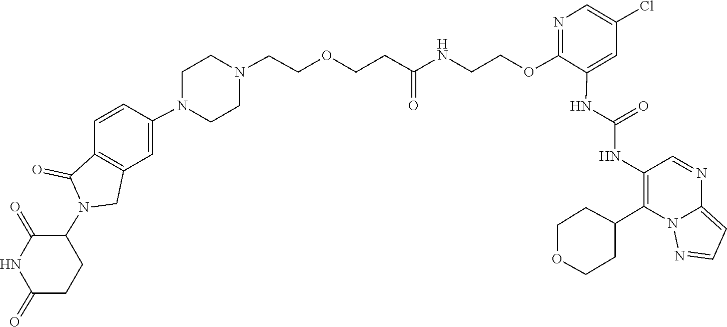 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCOCC1] N-[2-[[5-chloro-3-[(7-tetrahydropyran-4-ylpyrazolo[1,5-a]pyrimidin-6- yl)carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 43 | 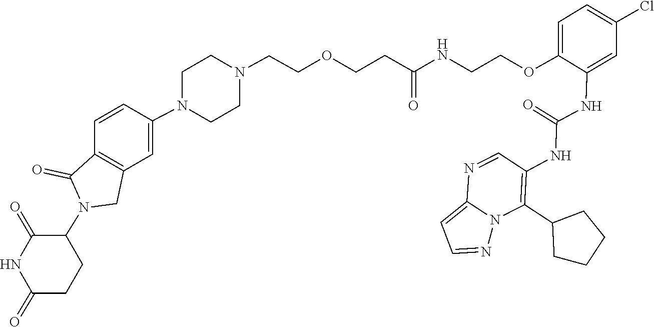 [SMILES: O=C(CCOCCN1CCN(c2ccc3c(c2)CN(C2CCC(=O)NC2=O)C3=O)CC1)NCCOc1ccc(Cl)cc1NC(=O)Nc1cnc2ccnn2c1C1CCCC1] N-[2-[4-chloro-2-[(7-cyclopentylpyrazolo[1,5-a]pyrimidin-6- yl)carbamoylamino]phenoxy]ethyl]-3-[2-[4-[2-(2,6-dioxo-3-piperidyl)-1-oxo- isoindolin-5-yl]piperazin-1-yl]ethoxy]propanamide |
| 44 | 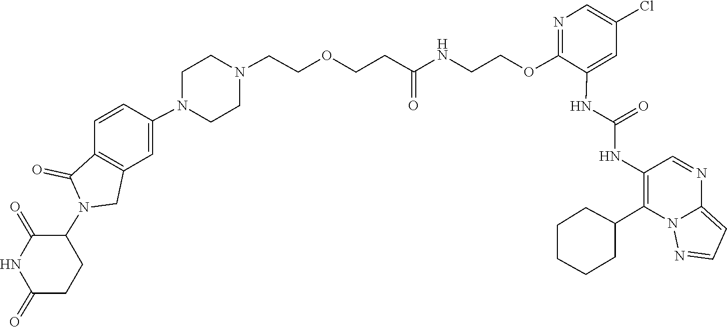 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCCC1] N-[2-[[5-chloro-3-[(7-cyclohexylpyrazolo[1,5-a]pyrimidin-6-yl)carbamoylamino]-2- pyridyl]oxylethyl]-3-[2-[4-[2-(2,6-dioxo-3-piperidyl)-1-oxo-isoindolin-5-yl]piperazin- 1-yl]ethoxy]propanamide |
| 45 | 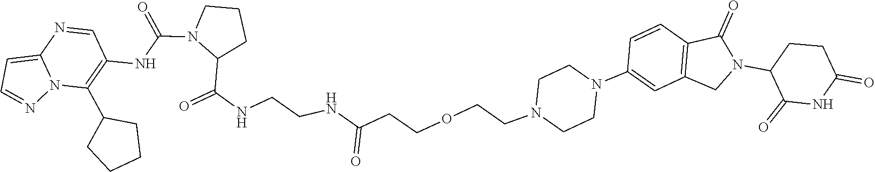 [SMILES: O=C(CCOCCN1CCN(c2ccc3c(c2)CN(C2CCC(=O)NC2=O)C3=O)CC1)NCCNC(=O)C1CCCN1C(=O)Nc1cnc2ccnn2c1C1CCCC1] N1-(7-cyclopentylpyrazolo[1,5-a]pyrimidin-6-yl)-N2-[2-[3-[2-[4-[2-(2,6-dioxo-3- piperidyl)-1-oxo-isoindolin-5-yl]piperazin-1- yl]ethoxy]propanoylamino]ethyl]pyrrolidine-1,2-dicarboxamide |
| 46 | 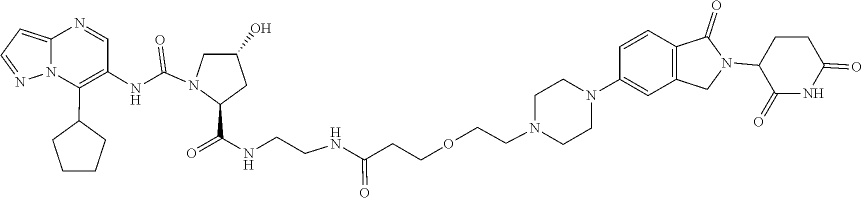 [SMILES: O=C(CCOCCN1CCN(c2ccc3c(c2)CN(C2CCC(=O)NC2=O)C3=O)CC1)NCCNC(=O)[C@@H]1C[C@@H](O)CN1C(=O)Nc1cnc2ccnn2c1C1CCCC1] (2S,4R)-N1-(7-cyclopentylpyrazolo[1,5-a]pyrimidin-6-yl)-N2-[2-[3-[2-[4-[2-(2,6- dioxo-3-piperidyl)-1-oxo-isoindolin-5-yl]piperazin-1- yl]ethoxy]propanoylamino]ethyl]-4-hydroxy-pyrrolidine-1,2-dicarboxamide |
| 47 | 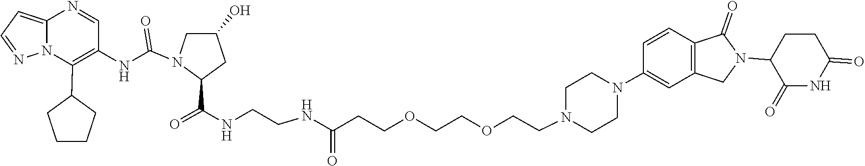 [SMILES: O=C(CCOCCOCCN1CCN(c2ccc3c(c2)CN(C2CCC(=O)NC2=O)C3=O)CC1)NCCNC(=O)[C@@H]1C[C@@H](O)CN1C(=O)Nc1cnc2ccnn2c1C1CCCC1] (2S,4R)-N1-(7-cyclopentylpyrazolo[1,5-a]pyrimidin-6-yl)-N2-[2-[3-[2-[2-[4-[2-(2,6- dioxo-3-piperidyl)-1-oxo-isoindolin-5-yl]piperazin-1- yl]ethoxy]ethoxy]propanoylamino]ethyl]-4-hydroxy-pyrrolidine-1,2-dicarboxamide |
| 48 | 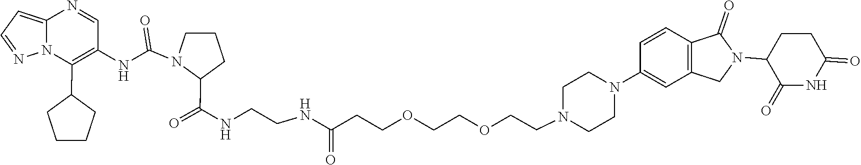 [SMILES: O=C(CCOCCOCCN1CCN(c2ccc3c(c2)CN(C2CCC(=O)NC2=O)C3=O)CC1)NCCNC(=O)C1CCCN1C(=O)Nc1cnc2ccnn2c1C1CCCC1] N1-(7-cyclopentylpyrazolo[1,5-a]pyrimidin-6-yl)-N2-[2-[3-[2-[2-[4-[2-(2,6-dioxo-3- piperidyl)-1-oxo-isoindolin-5-yl]piperazin-1- yl]ethoxy]ethoxy]propanoylamino]ethyl]pyrrolidine-1,2-dicarboxamide |
| 49 | 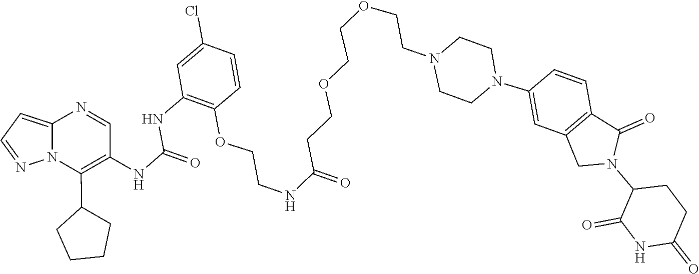 [SMILES: O=C(CCOCCOCCN1CCN(c2ccc3c(c2)CN(C2CCC(=O)NC2=O)C3=O)CC1)NCCOc1ccc(Cl)cc1NC(=O)Nc1cnc2ccnn2c1C1CCCC1] N-[2-[4-chloro-2-[(7-cyclopentylpyrazolo[1,5-a]pyrimidin-6- yl)carbamoylamino]phenoxy]ethyl]-3-[2-[2-[4-[2-(2,6-dioxo-3-piperidyl)-1-oxo- isoindolin-5-yl]piperazin-1-yl]ethoxy]ethoxy]propanamide |
| 50 | 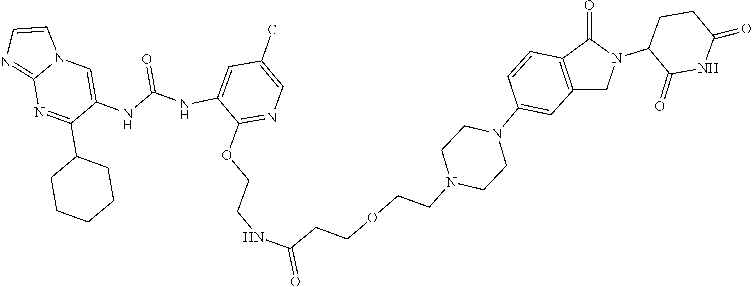 N-[2-[[5-chloro-3-[(7-cyclohexylimidazo[1,2-a]pyrimidin-6-yl)carbamoylamino]-2- pyridyl]oxy]ethyl]-3-[2-[4-[2-(2,6-dioxo-3-piperidyl)-1-oxo-isoindolin-5-yl]piperazin- 1-yl]ethoxy]propanamide |
| 51 | 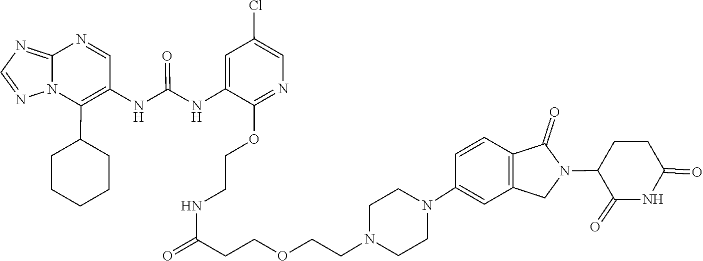 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ncnn2c1C1CCCCC1] N-[2-[[5-chloro-3-[(7-cyclohexyl-[1,2,4]triazolo[1,5-a]pyrimidin-6- yl)carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 52 | 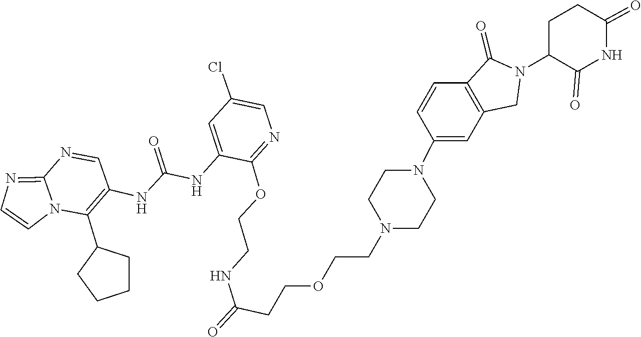 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2nccn2c1C1CCCC1] N-[2-[[5-chloro-3-[(5-cyclopentylimidazo[1,2-a]pyrimidin-6-yl)carbamoylamino]-2- pyridyl]oxy]ethyl]-3-[2-[4-[2-(2,6-dioxo-3-piperidyl)-1-oxo-isoindolin-5-yl]piperazin- 1-yl]ethoxy]propanamide |
| 53 | 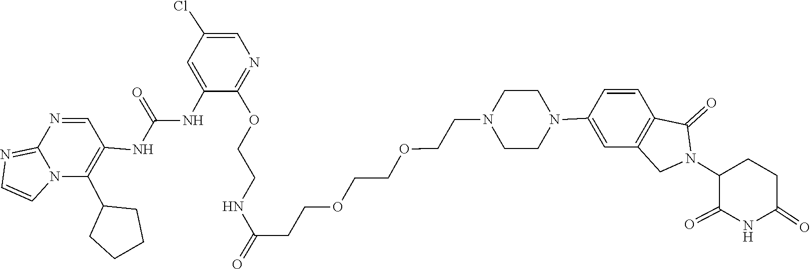 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2nccn2c1C1CCCC1] N-[2-[[5-chloro-3-[(5-cyclopentylimidazo[1,2-a]pyrimidin-6-yl)carbamoylamino]-2- pyridyl]oxy]ethyl]-3-[2-[2-[4-[2-(2,6-dioxo-3-piperidyl)-1-oxo-isoindolin-5- yl]piperazin-1-yl]ethoxy]ethoxy]propanamide |
| 54 | 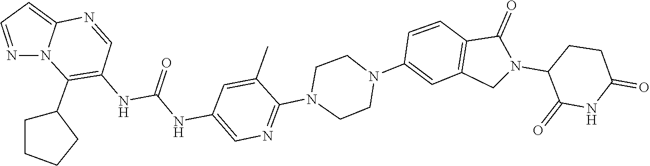 [SMILES: Cc1cc(NC(=O)Nc2cnc3ccnn3c2C2CCCC2)cnc1N1CCN(c2ccc3c(c2)CN(C2CCC(=O)NC2=O)C3=O)CC1] 1-(7-cyclopentylpyrazolo[1,5-a]pyrimidin-6-yl)-3-[6-[4-[2-(2,6-dioxo-3-piperidyl)-1- oxo-isoindolin-5-yl]piperazin-1-yl]-5-methyl-3-pyridyl]urea |
| 55 | 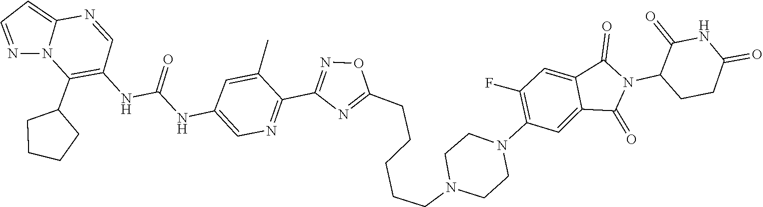 [SMILES: Cc1cc(NC(=O)Nc2cnc3ccnn3c2C2CCCC2)cnc1-c1noc(CCCCCN2CCN(c3cc4c(cc3F)C(=O)N(C3CCC(=O)NC3=O)C4=O)CC2)n1] 1-(7-cyclopentylpyrazolo[1,5-a]pyrimidin-6-yl)-3-[6-[5-[5-[4-[2-(2,6-dioxo-3- piperidyl)-6-fluoro-1,3-dioxo-isoindolin-5-yl]piperazin-1-yl]pentyl]-1,2,4-oxadiazol-3- yl]-5-methyl-3-pyridyl]urea |
| 56 | 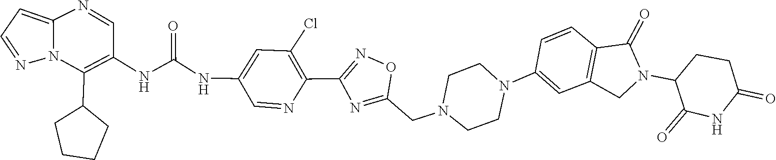 [SMILES: O=C1CCC(N2Cc3cc(N4CCN(Cc5nc(-c6ncc(NC(=O)Nc7cnc8ccnn8c7C7CCCC7)cc6Cl)no5)CC4)ccc3C2=O)C(=O)N1] 1-[5-chloro-6-[5-[[4-[2-(2,6-dioxo-3-piperidyl)-1-oxo-isoindolin-5-yl]piperazin-1- yl]methyl]-1,2,4-oxadiazol-3-yl]-3-pyridyl]-3-(7-cyclopentylpyrazolo[1,5-a]pyrimidin- 6-yl)urea |
| 57 | 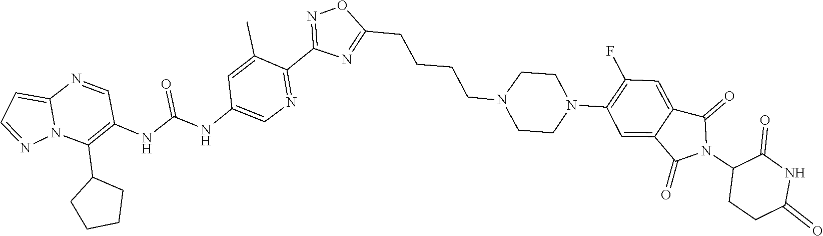 [SMILES: Cc1cc(NC(=O)Nc2cnc3ccnn3c2C2CCCC2)cnc1-c1noc(CCCCN2CCN(c3cc4c(cc3F)C(=O)N(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-6-fluoro-1,3-dioxo-isoindolin-5-yl]piperazin-1-yl]butyl]-1,2,4-oxadiazol-3- yl]-5-methyl-3-pyridyl]urea |
| 58 | 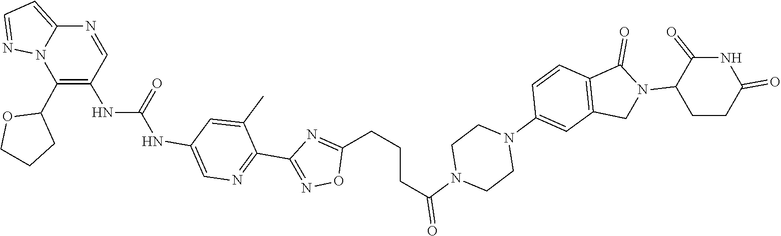 [SMILES: Cc1cc(NC(=O)Nc2cnc3ccnn3c2C2CCCO2)cnc1-c1noc(CCCC(=O)N2CCN(c3ccc4c(c3)CN(C3CCC(=O)NC3=O)C4=O)CC2)n1] 1-[6-[5-[4-[4-[2-(2,6-dioxo-3-piperidyl)-1-oxo-isoindolin-5-yl]piperazin-1-yl]-4-oxo- butyl]-1,2,4-oxadiazol-3-yl]-5-methyl-3-pyridyl]-3-(7-tetrahydrofuran-2- ylpyrazolo[1,5-a]pyrimidin-6-yl)urea |
| 59 | 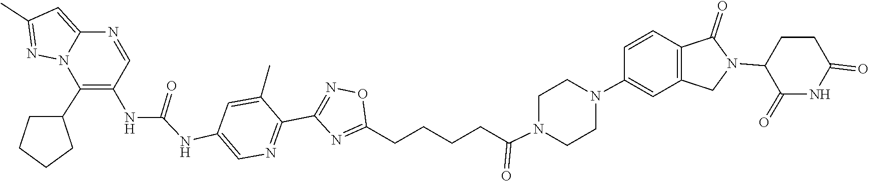 [SMILES: Cc1cc2ncc(NC(=O)Nc3cnc(-c4noc(CCCCC(=O)N5CCN(c6ccc7c(c6)CN(C6CCC(=O)NC6=O)C7=O)CC5)n4)c(C)c3)c(C3CCCC3)n2n1] 1-(7-cyclopentyl-2-methyl-pyrazolo[1,5-a]pyrimidin-6-yl)-3-[6-[5-[5-[4-[2-(2,6-dioxo- 3-piperidyl)-1-oxo-isoindolin-5-yl]piperazin-1-yl]-5-oxo-pentyl]-1,2,4-oxadiazol-3-yl]- 5-methyl-3-pyridyl]urea |
| 60 | 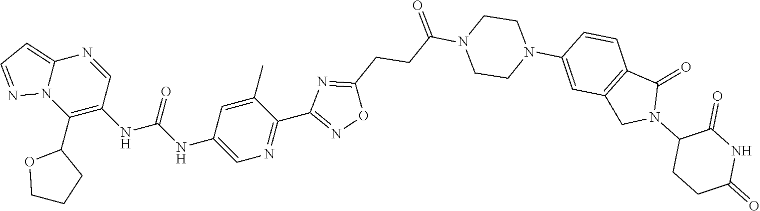 [SMILES: Cc1cc(NC(=O)Nc2cnc3ccnn3c2C2CCCO2)cnc1-c1noc(CCC(=O)N2CCN(c3ccc4c(c3)CN(C3CCC(=O)NC3=O)C4=O)CC2)n1] 1-[6-[5-[3-[4-[2-(2,6-dioxo-3-piperidyl)-1-oxo-isoindolin-5-yl]piperazin-1-yl]-3-oxo- propyl]-1,2,4-oxadiazol-3-yl]-5-methyl-3-pyridyl]-3-(7-tetrahydrofuran-2- ylpyrazolo[1,5-a]pyrimidin-6-yl)urea |
| 61 | 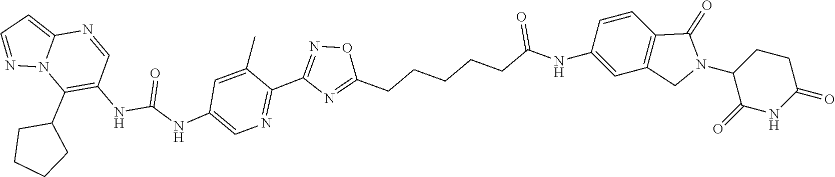 [SMILES: Cc1cc(NC(=O)Nc2cnc3ccnn3c2C2CCCC2)cnc1-c1noc(CCCCCC(=O)Nc2ccc3c(c2)CN(C2CCC(=O)NC2=O)C3=O)n1] 6-[3-[5-[(7-cyclopentylpyrazolo[1,5-a]pyrimidin-6-yl)carbamoylamino]-3-methyl-2- pyridyl]-1,2,4-oxadiazol-5-yl]-N-[2-(2,6-dioxo-3-piperidyl)-1-oxo-isoindolin-5- yl]hexanamide |
| 62 | 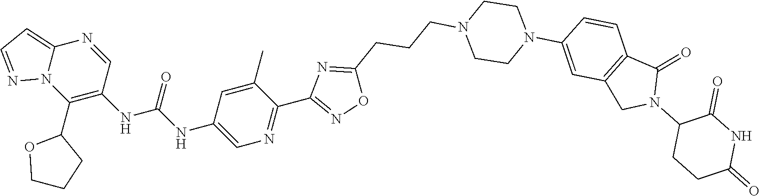 [SMILES: Cc1cc(NC(=O)Nc2cnc3ccnn3c2C2CCCO2)cnc1-c1noc(CCCN2CCN(c3ccc4c(c3)CN(C3CCC(=O)NC3=O)C4=O)CC2)n1] 1-[6-[5-[3-[4-[2-(2,6-dioxo-3-piperidyl)-1-oxo-isoindolin-5-yl]piperazin-1-yl]propyl]- 1,2,4-oxadiazol-3-yl]-5-methyl-3-pyridyl]-3-(7-tetrahydrofuran-2-ylpyrazolo[1,5- a]pyrimidin-6-yl)urea |
| 63 | 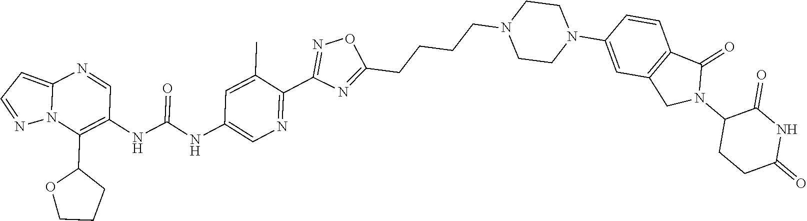 [SMILES: Cc1cc(NC(=O)Nc2cnc3ccnn3c2C2CCCO2)cnc1-c1noc(CCCCN2CCN(c3ccc4c(c3)CN(C3CCC(=O)NC3=O)C4=O)CC2)n1] 1-[6-[5-[4-[4-[2-(2,6-dioxo-3-piperidyl)-1-oxo-isoindolin-5-yl]piperazin-1-yl]butyl]- 1,2,4-oxadiazol-3-yl]-5-methyl-3-pyridyl]-3-(7-tetrahydrofuran-2-ylpyrazolo[1,5- a]pyrimidin-6-yl)urea |
| 64 | 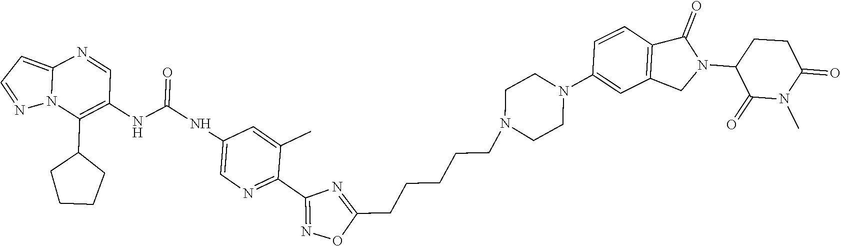 [SMILES: Cc1cc(NC(=O)Nc2cnc3ccnn3c2C2CCCC2)cnc1-c1noc(CCCCCN2CCN(c3ccc4c(c3)CN(C3CCC(=O)N(C)C3=O)C4=O)CC2)n1] 1-(7-cyclopentylpyrazolo[1,5-a]pyrimidin-6-yl)-3-[5-methyl-6-[5-[5-[4-[2-(1-methyl- 2,6-dioxo-3-piperidyl)-1-oxo-isoindolin-5-yl]piperazin-1-yl]pentyl]-1,2,4-oxadiazol-3- yl]-3-pyridyl]urea |
| 65 | 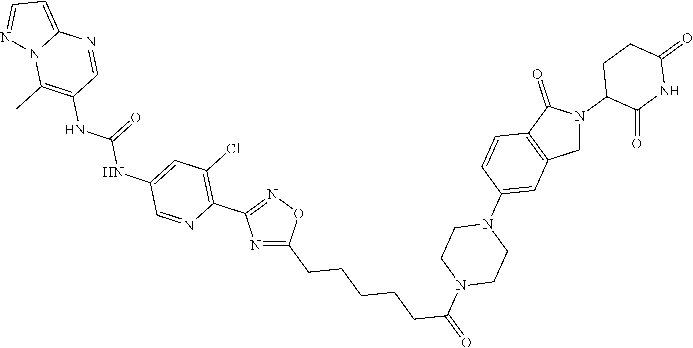 [SMILES: Cc1c(NC(=O)Nc2cnc(-c3noc(CCCCCC(=O)N4CCN(c5ccc6c(c5)CN(C5CCC(=O)NC5=O)C6=O)CC4)n3)c(Cl)c2)cnc2ccnn12] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-methylpyrazolo[1,5- a]pyrimidin-6-yl)urea |
| 66 | 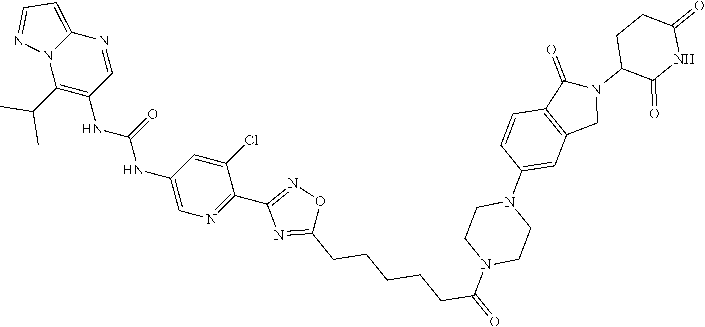 [SMILES: CC(C)c1c(NC(=O)Nc2cnc(-c3noc(CCCCCC(=O)N4CCN(c5ccc6c(c5)CN(C5CCC(=O)NC5=O)C6=O)CC4)n3)c(Cl)c2)cnc2ccnn12] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-isopropylpyrazolo[1,5- a]pyrimidin-6-yl)urea |
| 67 | 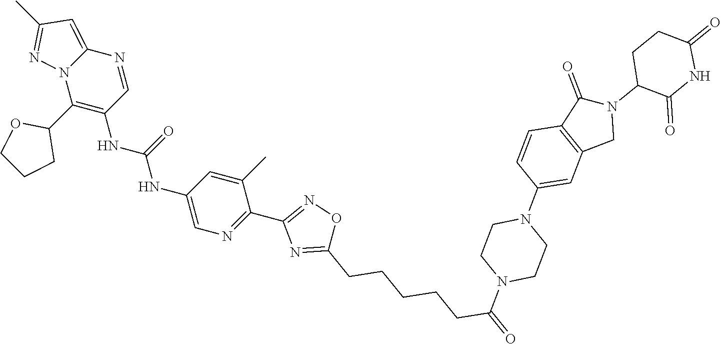 [SMILES: Cc1cc2ncc(NC(=O)Nc3cnc(-c4noc(CCCCCC(=O)N5CCN(c6ccc7c(c6)CN(C6CCC(=O)NC6=O)C7=O)CC5)n4)c(C)c3)c(C3CCCO3)n2n1] 1-[6-[5-[6-[4-[2-(2,6-dioxo-3-piperidyl)-1-oxo-isoindolin-5-yl]piperazin-1-yl]-6-oxo- hexyl]-1,2,4-oxadiazol-3-yl]-5-methyl-3-pyridyl]-3-(2-methyl-7-tetrahydrofuran-2-yl- pyrazolo[1,5-a]pyrimidin-6-yl)urea |
| 68 | 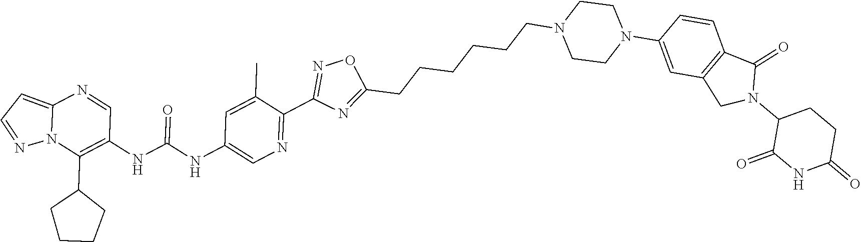 [SMILES: Cc1cc(NC(=O)Nc2cnc3ccnn3c2C2CCCC2)cnc1-c1noc(CCCCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-methyl- 3-pyridyl]urea |
| 69 | 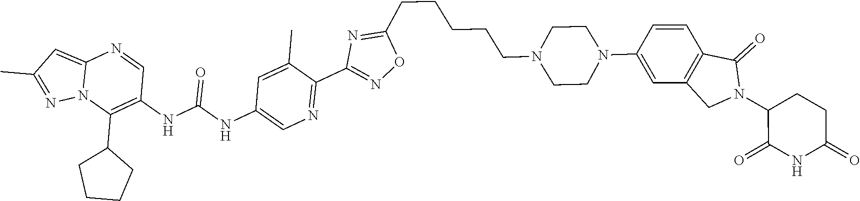 [SMILES: Cc1cc2ncc(NC(=O)Nc3cnc(-c4noc(CCCCCN5CCN(c6ccc7c(c6)CN(C6CCC(=O)NC6=O)C7=O)CC5)n4)c(C)c3)c(C3CCCC3)n2n1] 1-(7-cyclopentyl-2-methyl-pyrazolo[1,5-a]pyrimidin-6-yl)-3-[6-[5-[5-[4-[2-(2,6-dioxo- 3-piperidyl)-1-oxo-isoindolin-5-yl]piperazin-1-yl]pentyl]-1,2,4-oxadiazol-3-yl]-5- methyl-3-pyridyl]urea |
| 70 | 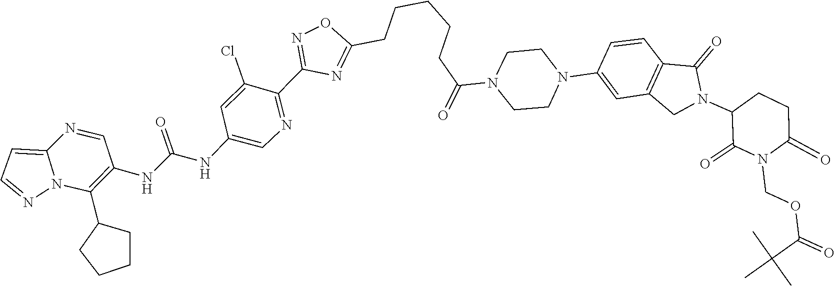 [SMILES: CC(C)(C)C(=O)OCN1C(=O)CCC(N2Cc3cc(N4CCN(C(=O)CCCCCc5nc(-c6ncc(NC(=O)Nc7cnc8ccnn8c7C7CCCC7)cc6Cl)no5)CC4)ccc3C2=O)C1=O] [3-[5-[4-[6-[3-[3-chloro-5-[(7-cyclopentylpyrazolo[1,5-a]pyrimidin-6- yl)carbamoylamino]-2-pyridyl]-1,2,4-oxadiazol-5-yl]hexanoyl]piperazin-1-yl]-1-oxo- isoindolin-2-yl]-2,6-dioxo-1-piperidyl]methyl 2,2-dimethylpropanoate |
| 71 | 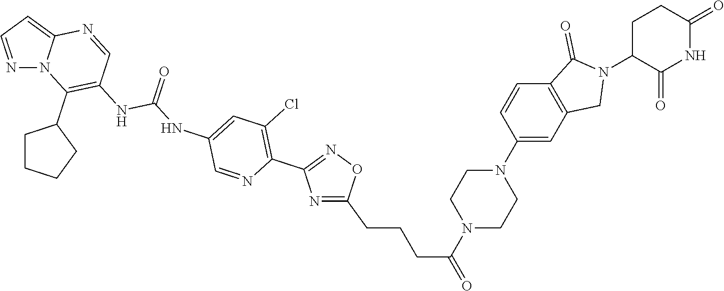 [SMILES: O=C1CCC(N2Cc3cc(N4CCN(C(=O)CCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]-4-oxo-butyl]-1,2,4-oxadiazol-3-yl]-3-pyridyl]-3-(7-cyclopentylpyrazolo[1,5- a]pyrimidin-6-yl)urea |
| 72 | 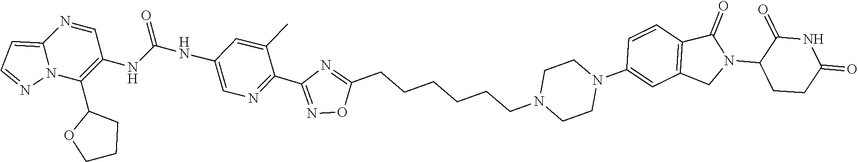 [SMILES: Cc1cc(NC(=O)Nc2cnc3ccnn3c2C2CCCO2)cnc1-c1noc(CCCCCCN2CCN(c3ccc4c(c3)CN(C3CCC(=O)NC3=O)C4=O)CC2)n1] 1-[6-[5-[6-[4-[2-(2,6-dioxo-3-piperidyl)-1-oxo-isoindolin-5-yl]piperazin-1-yl]hexyl]- 1,2,4-oxadiazol-3-yl]-5-methyl-3-pyridyl]-3-(7-tetrahydrofuran-2-ylpyrazolo[1,5- a]pyrimidin-6-yl)urea |
| 73 | 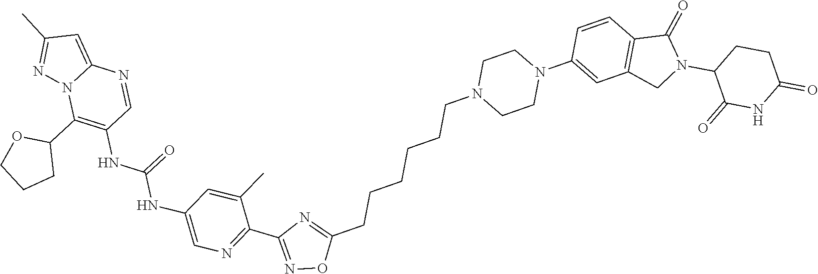 [SMILES: Cc1cc2ncc(NC(=O)Nc3cnc(-c4noc(CCCCCCN5CCN(c6ccc7c(c6)CN(C6CCC(=O)NC6=O)C7=O)CC5)n4)c(C)c3)c(C3CCCO3)n2n1] 1-[6-[5-[6-[4-[2-(2,6-dioxo-3-piperidyl)-1-oxo-isoindolin-5-yl]piperazin-1-yl]hexyl]- 1,2,4-oxadiazol-3-yl]-5-methyl-3-pyridyl]-3-(2-methyl-7-tetrahydrofuran-2-yl- pyrazolo[1,5-a]pyrimidin-6-yl)urea |
| 74 | 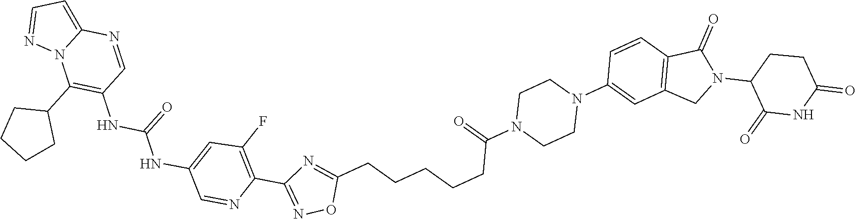 [SMILES: O=C1CCC(N2Cc3cc(N4CCN(C(=O)CCCCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- fluoro-3-pyridyl]urea |
| 75 | 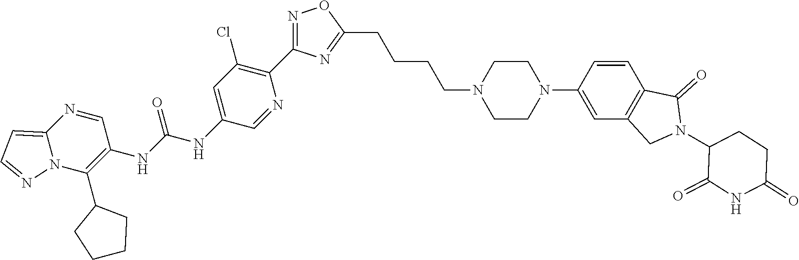 [SMILES: O=C1CCC(N2Cc3cc(N4CCN(CCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]butyl]-1,2,4-oxadiazol-3-yl]-3-pyridyl]-3-(7-cyclopentylpyrazolo[1,5-a]pyrimidin-6- yl)urea |
| 76 | 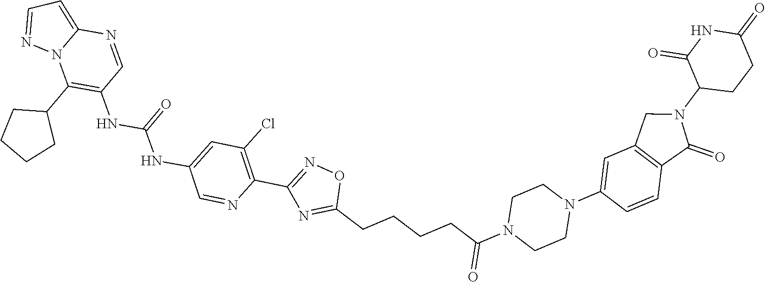 [SMILES: O=C1CCC(N2Cc3cc(N4CCN(C(=O)CCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]-5-oxo-pentyl]-1,2,4-oxadiazol-3-yl]-3-pyridyl]-3-(7-cyclopentylpyrazolo[1,5- a]pyrimidin-6-yl)urea |
| 77 | 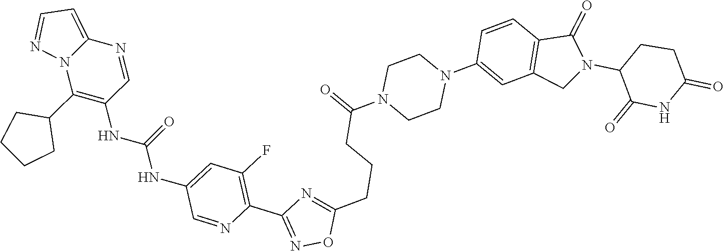 [SMILES: O=C1CCC(N2Cc3cc(N4CCN(C(=O)CCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- fluoro-3-pyridyl]urea |
| 78 | 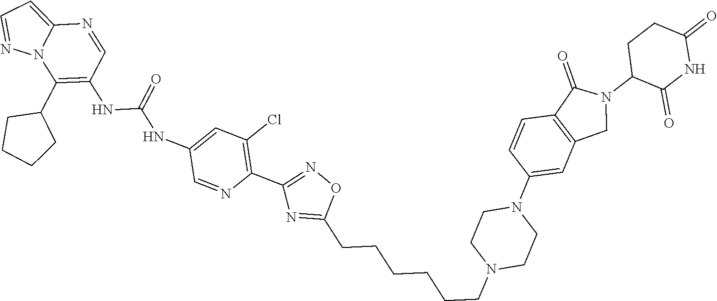 [SMILES: O=C1CCC(N2Cc3cc(N4CCN(CCCCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-cyclopentylpyrazolo[1,5-a]pyrimidin- 6-yl)urea |
| 79 | 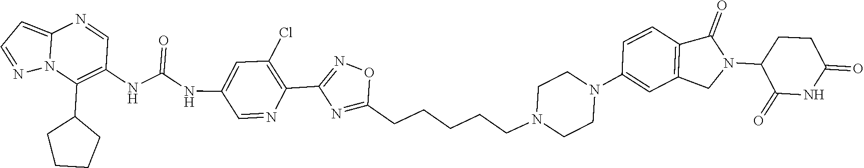 [SMILES: O=C1CCC(N2Cc3cc(N4CCN(CCCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]pentyl]-1,2,4-oxadiazol-3-yl]-3-pyridyl]-3-(7-cyclopentylpyrazolo[1,5-a]pyrimidin- 6-yl)urea |
| 80 | 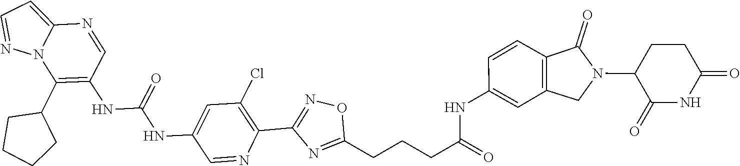 [SMILES: O=C1CCC(N2Cc3cc(NC(=O)CCCc4nc(-c5ncc(NC(=O)Nc6cnc7ccnn7c6C6CCCC6)cc5Cl)no4)ccc3C2=O)C(=O)N1] 4-[3-[3-chloro-5-[(7-cyclopentylpyrazolo[1,5-a]pyrimidin-6-yl)carbamoylamino]-2- pyridyl]-1,2,4-oxadiazol-5-yl]-N-[2-(2,6-dioxo-3-piperidyl)-1-oxo-isoindolin-5- yl]butanamide |
| 81 | 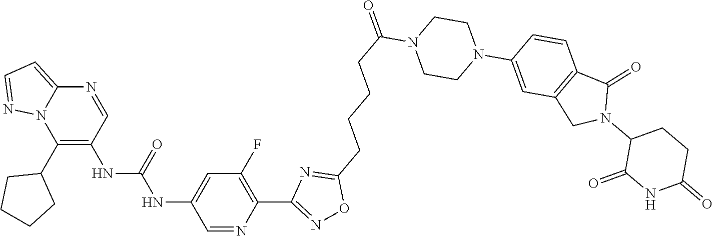 [SMILES: O=C1CCC(N2Cc3cc(N4CCN(C(=O)CCCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- fluoro-3-pyridyl]urea |
| 82 | 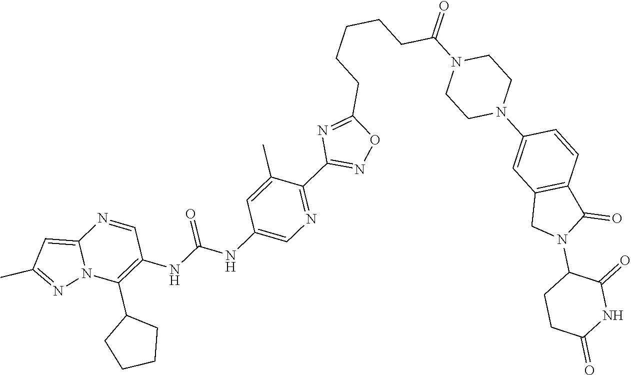 [SMILES: Cc1cc2ncc(NC(=O)Nc3cnc(-c4noc(CCCCCC(=O)N5CCN(c6ccc7c(c6)CN(C6CCC(=O)NC6=O)C7=O)CC5)n4)c(C)c3)c(C3CCCC3)n2n1] 1-(7-cyclopentyl-2-methyl-pyrazolo[1,5-a]pyrimidin-6-yl)-3-[6-[5-[6-[4-[2-(2,6-dioxo- 3-piperidyl)-1-oxo-isoindolin-5-yl]piperazin-1-yl]-6-oxo-hexyl]-1,2,4-oxadiazol-3-yl]- 5-methyl-3-pyridyl]urea |
| 83 | 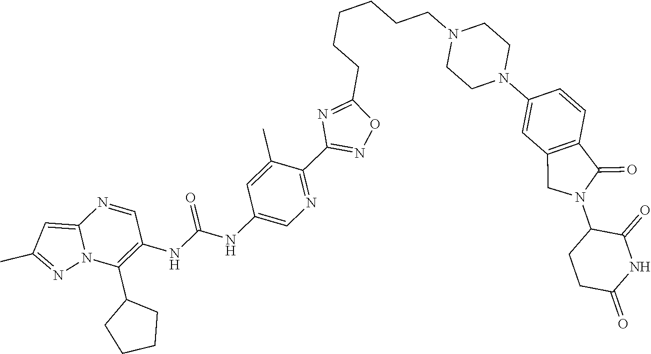 [SMILES: Cc1cc2ncc(NC(=O)Nc3cnc(-c4noc(CCCCCCN5CCN(c6ccc7c(c6)CN(C6CCC(=O)NC6=O)C7=O)CC5)n4)c(C)c3)c(C3CCCC3)n2n1] 1-(7-cyclopentyl-2-methyl-pyrazolo[1,5-a]pyrimidin-6-yl)-3-[6-[5-[6-[4-[2-(2,6-dioxo- 3-piperidyl)-1-oxo-isoindolin-5-yl]piperazin-1-yl]hexyl]-1,2,4-oxadiazol-3-yl]-5- methyl-3-pyridyl]urea |
| 84 | 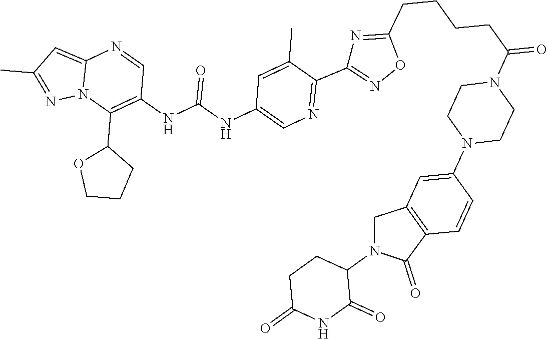 [SMILES: Cc1cc2ncc(NC(=O)Nc3cnc(-c4noc(CCCCC(=O)N5CCN(c6ccc7c(c6)CN(C6CCC(=O)NC6=O)C7=O)CC5)n4)c(C)c3)c(C3CCCO3)n2n1] 1-[6-[5-[5-[4-[2-(2,6-dioxo-3-piperidyl)-1-oxo-isoindolin-5-yl]piperazin-1-yl]-5-oxo- pentyl]-1,2,4-oxadiazol-3-yl]-5-methyl-3-pyridyl]-3-(2-methyl-7-tetrahydrofuran-2-yl- pyrazolo[1,5-a]pyrimidin-6-yl)urea |
| 85 | 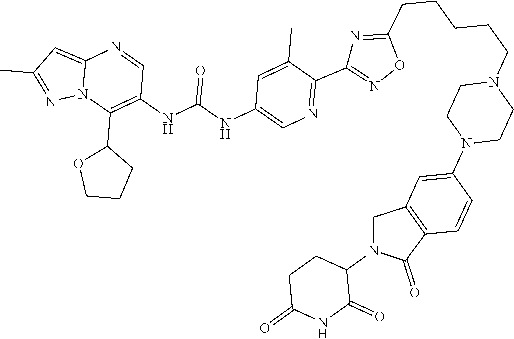 [SMILES: Cc1cc2ncc(NC(=O)Nc3cnc(-c4noc(CCCCCN5CCN(c6ccc7c(c6)CN(C6CCC(=O)NC6=O)C7=O)CC5)n4)c(C)c3)c(C3CCCO3)n2n1] 1-[6-[5-[5-[4-[2-(2,6-dioxo-3-piperidyl)-1-oxo-isoindolin-5-yl]piperazin-1-yl]pentyl]- 1,2,4-oxadiazol-3-yl]-5-methyl-3-pyridyl]-3-(2-methyl-7-tetrahydrofuran-2-yl- pyrazolo[1,5-a]pyrimidin-6-yl)urea |
| 86 | 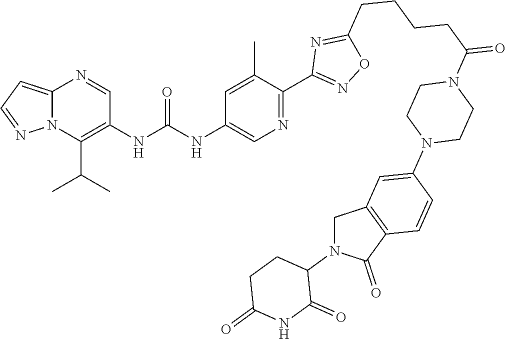 [SMILES: Cc1cc(NC(=O)Nc2cnc3ccnn3c2C(C)C)cnc1-c1noc(CCCCC(=O)N2CCN(c3ccc4c(c3)CN(C3CCC(=O)NC3=O)C4=O)CC2)n1] 1-[6-[5-[5-[4-[2-(2,6-dioxo-3-piperidyl)-1-oxo-isoindolin-5-yl]piperazin-1-yl]-5-oxo- pentyl]-1,2,4-oxadiazol-3-yl]-5-methyl-3-pyridyl]-3-(7-isopropylpyrazolo[1,5- a]pyrimidin-6-yl)urea |
| 87 | 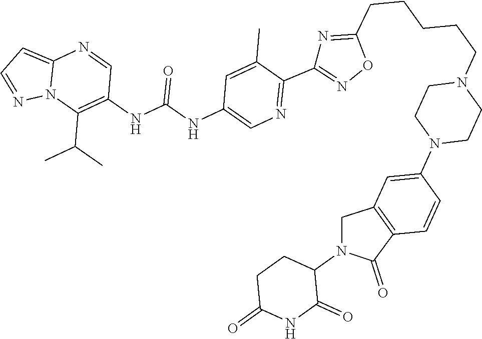 [SMILES: Cc1cc(NC(=O)Nc2cnc3ccnn3c2C(C)C)cnc1-c1noc(CCCCCN2CCN(c3ccc4c(c3)CN(C3CCC(=O)NC3=O)C4=O)CC2)n1] 1-[6-[5-[5-[4-[2-(2,6-dioxo-3-piperidyl)-1-oxo-isoindolin-5-yl]piperazin-1-yl]pentyl]- 1,2,4-oxadiazol-3-yl]-5-methyl-3-pyridyl]-3-(7-isopropylpyrazolo[1,5-a]pyrimidin-6- yl)urea |
| 88 | 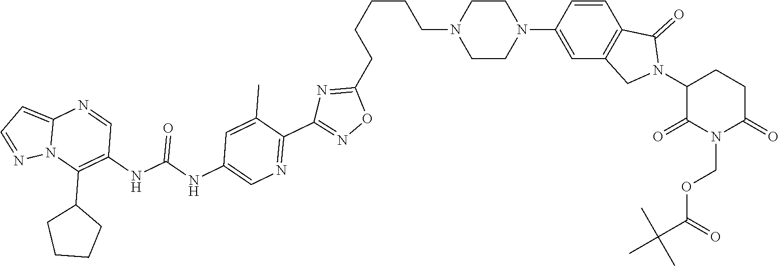 [SMILES: Cc1cc(NC(=O)Nc2cnc3ccnn3c2C2CCCC2)cnc1-c1noc(CCCCCN2CCN(c3ccc4c(c3)CN(C3CCC(=O)N(COC(=O)C(C)(C)C)C3=O)C4=O)CC2)n1] [3-[5-[4-[5-[3-[5-[(7-cyclopentylpyrazolo[1,5-a]pyrimidin-6-yl)carbamoylamino]-3- methyl-2-pyridyl]-1,2,4-oxadiazol-5-yl]pentyl]piperazin-1-yl]-1-oxo-isoindolin-2-yl]- 2,6-dioxo-1-piperidyl]methyl 2,2-dimethylpropanoate |
| 89 | 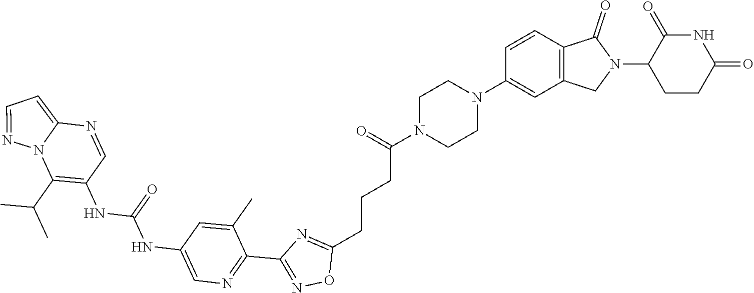 [SMILES: Cc1cc(NC(=O)Nc2cnc3ccnn3c2C(C)C)cnc1-c1noc(CCCC(=O)N2CCN(c3ccc4c(c3)CN(C3CCC(=O)NC3=O)C4=O)CC2)n1] 1-[6-[5-[4-[4-[2-(2,6-dioxo-3-piperidyl)-1-oxo-isoindolin-5-yl]piperazin-1-yl]-4-oxo- butyl]-1,2,4-oxadiazol-3-yl]-5-methyl-3-pyridyl]-3-(7-isopropylpyrazolo[1,5- a]pyrimidin-6-yl)urea |
| 90 | 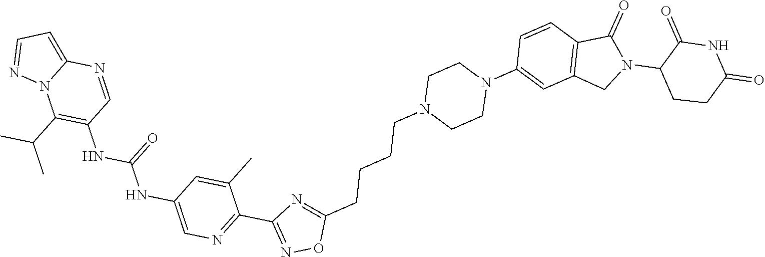 [SMILES: Cc1cc(NC(=O)Nc2cnc3ccnn3c2C(C)C)cnc1-c1noc(CCCCN2CCN(c3ccc4c(c3)CN(C3CCC(=O)NC3=O)C4=O)CC2)n1] 1-[6-[5-[4-[4-[2-(2,6-dioxo-3-piperidyl)-1-oxo-isoindolin-5-yl]piperazin-1-yl]butyl]- 1,2,4-oxadiazol-3-yl]-5-methyl-3-pyridyl]-3-(7-isopropylpyrazolo[1,5-a]pyrimidin-6- yl)urea |
| 91 | 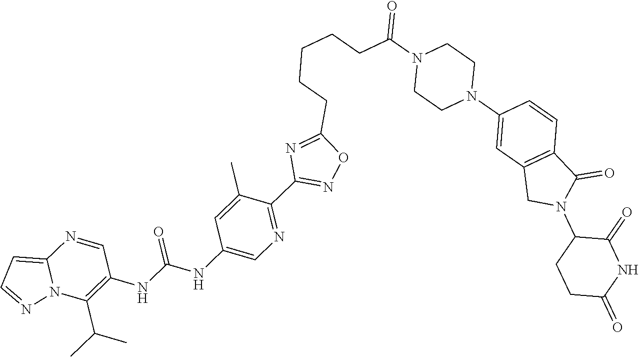 [SMILES: Cc1cc(NC(=O)Nc2cnc3ccnn3c2C(C)C)cnc1-c1noc(CCCCCC(=O)N2CCN(c3ccc4c(c3)CN(C3CCC(=O)NC3=O)C4=O)CC2)n1] 1-[6-[5-[6-[4-[2-(2,6-dioxo-3-piperidyl)-1-oxo-isoindolin-5-yl]piperazin-1-yl]-6-oxo- hexyl]-1,2,4-oxadiazol-3-yl]-5-methyl-3-pyridyl]-3-(7-isopropylpyrazolo[1,5- a]pyrimidin-6-yl)urea |
| 92 | 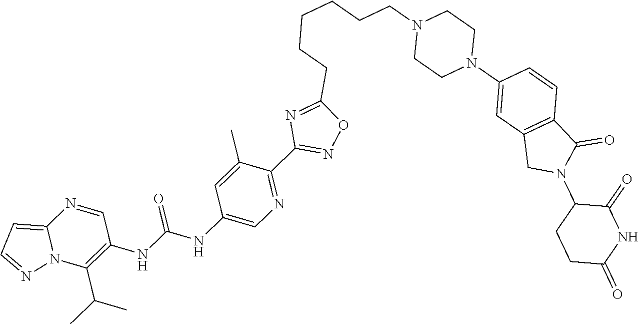 [SMILES: Cc1cc(NC(=O)Nc2cnc3ccnn3c2C(C)C)cnc1-c1noc(CCCCCCN2CCN(c3ccc4c(c3)CN(C3CCC(=O)NC3=O)C4=O)CC2)n1] 1-[6-[5-[6-[4-[2-(2,6-dioxo-3-piperidyl)-1-oxo-isoindolin-5-yl]piperazin-1-yl]hexyl]- 1,2,4-oxadiazol-3-yl]-5-methyl-3-pyridyl]-3-(7-isopropylpyrazolo[1,5-a]pyrimidin-6- yl)urea |
| 93 | 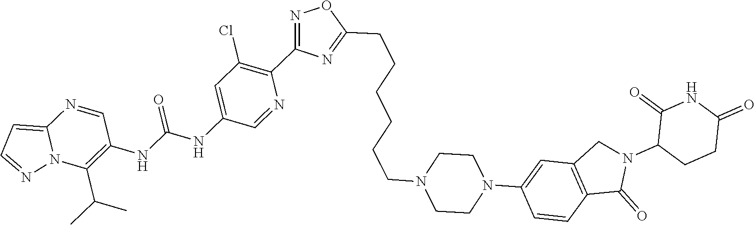 [SMILES: CC(C)c1c(NC(=O)Nc2cnc(-c3noc(CCCCCCN4CCN(c5ccc6c(c5)CN(C5CCC(=O)NC5=O)C6=O)CC4)n3)c(Cl)c2)cnc2ccnn12] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-isopropylpyrazolo[1,5-a]pyrimidin-6- yl)urea |
| 94 | 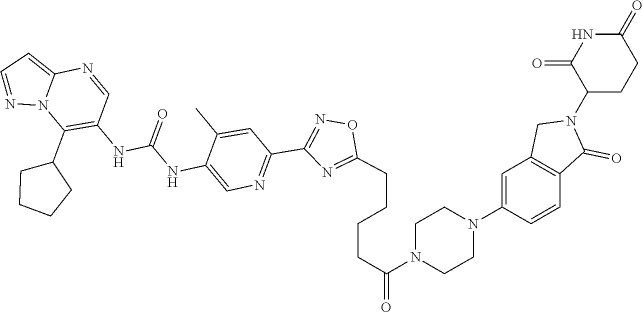 [SMILES: Cc1cc(-c2noc(CCCCC(=O)N3CCN(c4ccc5c(c4)CN(C4CCC(=O)NC4=O)C5=O)CC3)n2)ncc1NC(=O)Nc1cnc2ccnn2c1C1CCCC1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-4- methyl-3-pyridyl]urea |
| 95 | 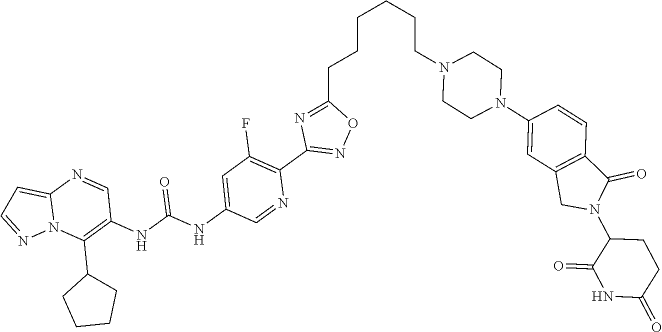 [SMILES: O=C1CCC(N2Cc3cc(N4CCN(CCCCCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-fluoro- 3-pyridyl]urea |
| 96 | 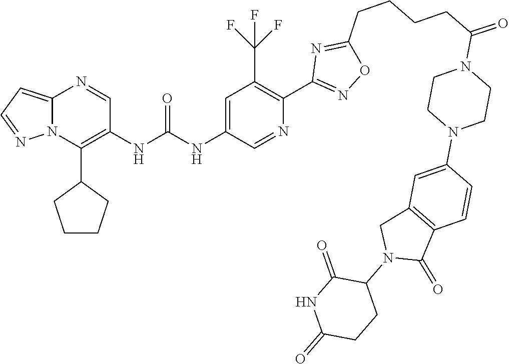 [SMILES: O=C1CCC(N2Cc3cc(N4CCN(C(=O)CCCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- (trifluoromethyl)-3-pyridyl]urea |
| 97 | 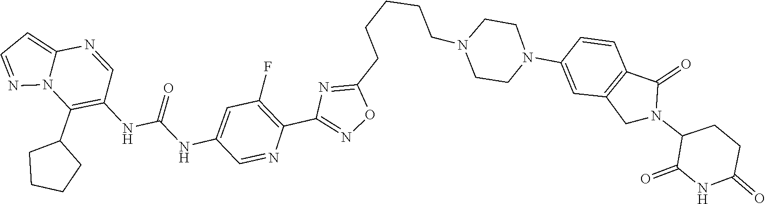 [SMILES: O=C1CCC(N2Cc3cc(N4CCN(CCCCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5-fluoro- 3-pyridyl]urea |
| 98 | 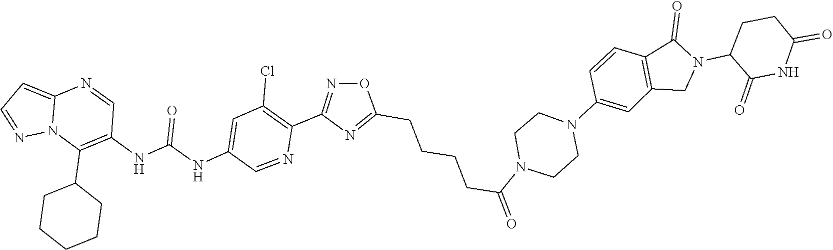 [SMILES: O=C1CCC(N2Cc3cc(N4CCN(C(=O)CCCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]-5-oxo-pentyl]-1,2,4-oxadiazol-3-yl]-3-pyridyl]-3-(7-cyclohexylpyrazolo[1,5- a]pyrimidin-6-yl)urea |
| 99 | 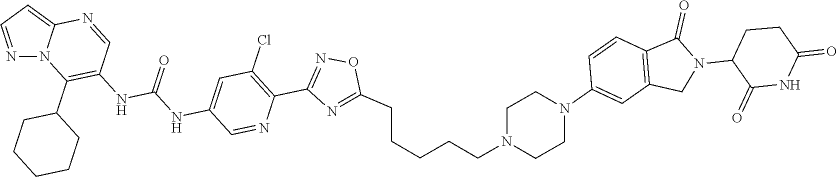 [SMILES: O=C1CCC(N2Cc3cc(N4CCN(CCCCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]pentyl]-1,2,4-oxadiazol-3-yl]-3-pyridyl]-3-(7-cyclohexylpyrazolo[1,5-a]pyrimidin- 6-yl)urea |
| 100 | 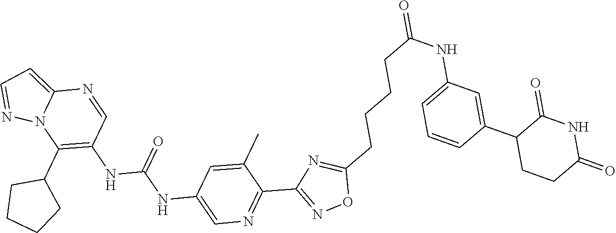 [SMILES: Cc1cc(NC(=O)Nc2cnc3ccnn3c2C2CCCC2)cnc1-c1noc(CCCCC(=O)Nc2cccc(C3CCC(=O)NC3=O)c2)n1] 5-[3-[5-[(7-cyclopentylpyrazolo[1,5-a]pyrimidin-6-yl)carbamoylamino]-3-methyl-2- pyridyl]-1,2,4-oxadiazol-5-yl]-N-[3-(2,6-dioxo-3-piperidyl)phenyl]pentanamide |
| 101 | 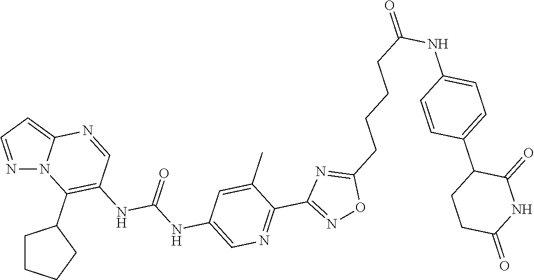 [SMILES: Cc1cc(NC(=O)Nc2cnc3ccnn3c2C2CCCC2)cnc1-c1noc(CCCCC(=O)Nc2ccc(C3CCC(=O)NC3=O)cc2)n1] 5-[3-[5-[(7-cyclopentylpyrazolo[1,5-a]pyrimidin-6-yl)carbamoylamino]-3-methyl-2- pyridyl]-1,2,4-oxadiazol-5-yl]-N-[4-(2,6-dioxo-3-piperidyl)phenyl]pentanamide |
| 102 | 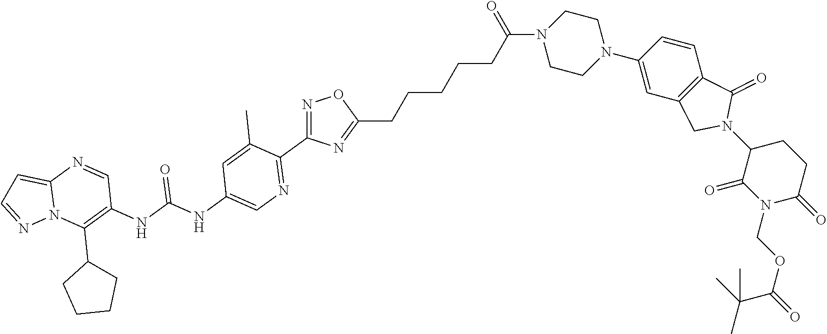 [SMILES: Cc1cc(NC(=O)Nc2cnc3ccnn3c2C2CCCC2)cnc1-c1noc(CCCCCC(=O)N2CCN(c3ccc4c(c3)CN(C3CCC(=O)N(COC(=O)C(C)(C)C)C3=O)C4=O)CC2)n1] [3-[5-[4-[6-[3-[5-[(7-cyclopentylpyrazolo[1,5-a]pyrimidin-6-yl)carbamoylamino]-3- methyl-2-pyridyl]-1,2,4-oxadiazol-5-yl]hexanoyl]piperazin-1-yl]-1-oxo-isoindolin-2- yl]-2,6-dioxo-1-piperidyl]methyl 2,2-dimethylpropanoate |
| 103 | 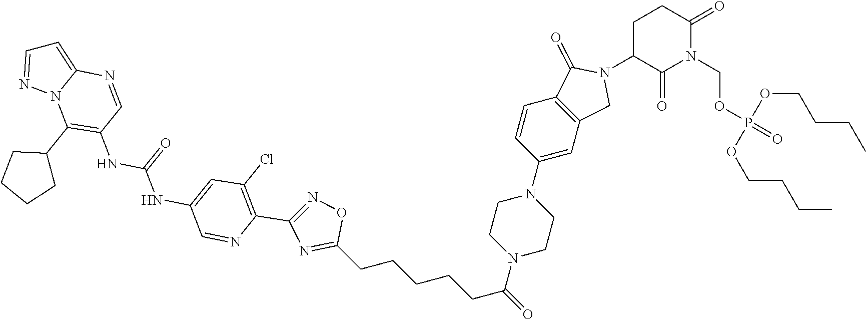 [SMILES: CCCCOP(=O)(OCCCC)OCN1C(=O)CCC(N2Cc3cc(N4CCN(C(=O)CCCCCc5nc(-c6ncc(NC(=O)Nc7cnc8ccnn8c7C7CCCC7)cc6Cl)no5)CC4)ccc3C2=O)C1=O] dibutyl [3-[5-[4-[6-[3-[3-chloro-5-[(7-cyclopentylpyrazolo[1,5-a]pyrimidin-6- yl)carbamoylamino]-2-pyridyl]-1,2,4-oxadiazol-5-yl]hexanoyl]piperazin-1-yl]-1-oxo- isoindolin-2-yl]-2,6-dioxo-1-piperidyl]methyl phosphate |
| 104 | 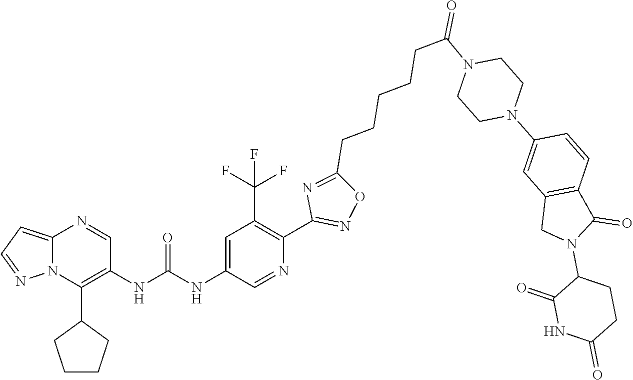 [SMILES: O=C1CCC(N2Cc3cc(N4CCN(C(=O)CCCCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- (trifluoromethyl)-3-pyridyl]urea |
| 105 | 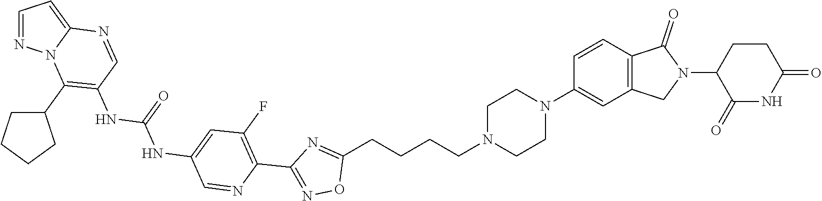 [SMILES: O=C1CCC(N2Cc3cc(N4CCN(CCCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butyl]-1,2,4-oxadiazol-3-yl]-5-fluoro- 3-pyridyl]urea |
| 106 | 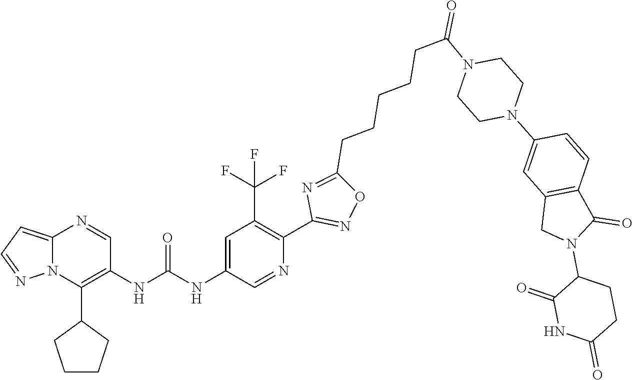 [SMILES: O=C1CCC(N2Cc3cc(N4CCN(C(=O)CCCCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- (trifluoromethyl)-3-pyridyl]urea |
| 107 | 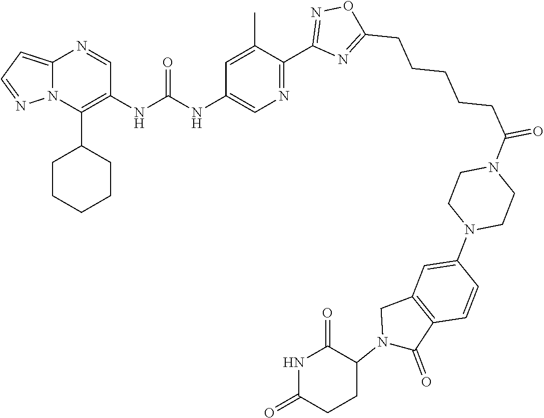 [SMILES: Cc1cc(NC(=O)Nc2cnc3ccnn3c2C2CCCCC2)cnc1-c1noc(CCCCCC(=O)N2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 108 | 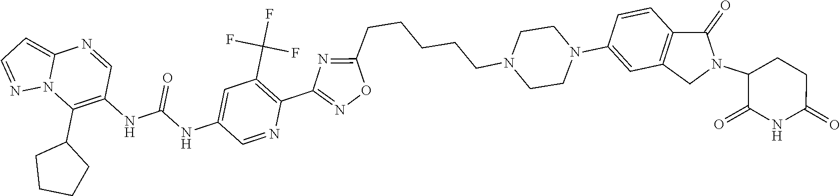 [SMILES: O=C1CCC(N2Cc3cc(N4CCN(CCCCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- (trifluoromethyl)-3-pyridyl]urea |
| 109 | 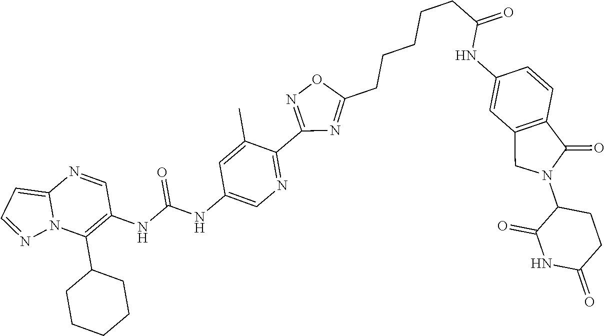 [SMILES: Cc1cc(NC(=O)Nc2cnc3ccnn3c2C2CCCCC2)cnc1-c1noc(CCCCCC(=O)Nc2ccc3c(c2)CN(C2CCC(=O)NC2=O)C3=O)n1] 6-[3-[5-[(7-cyclohexylpyrazolo[1,5-a]pyrimidin-6-yl)carbamoylamino]-3-methyl-2- pyridyl]-1,2,4-oxadiazol-5-yl]-N-[2-(2,6-dioxo-3-piperidyl)-1-oxo-isoindolin-5- yl]hexanamide |
| 110 | 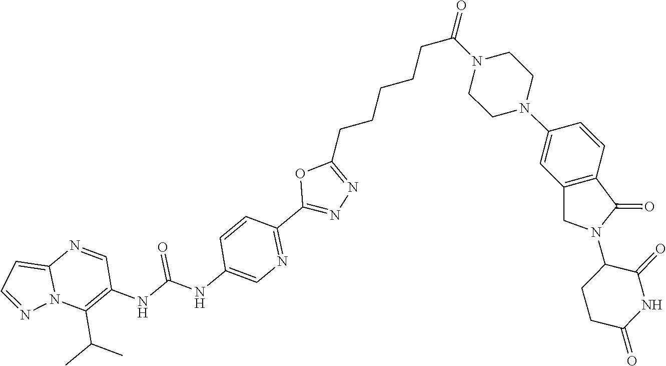 [SMILES: CC(C)c1c(NC(=O)Nc2ccc(-c3nnc(CCCCCC(=O)N4CCN(c5ccc6c(c5)CN(C5CCC(=O)NC5=O)C6=O)CC4)o3)nc2)cnc2ccnn12] 1-[6-[5-[6-[4-[2-(2,6-dioxo-3-piperidyl)-1-oxo-isoindolin-5-yl]piperazin-1-yl]-6-oxo- hexyl]-1,3,4-oxadiazol-2-yl]-3-pyridyl]-3-(7-isopropylpyrazolo[1,5-a]pyrimidin-6- yl)urea |
| 111 | 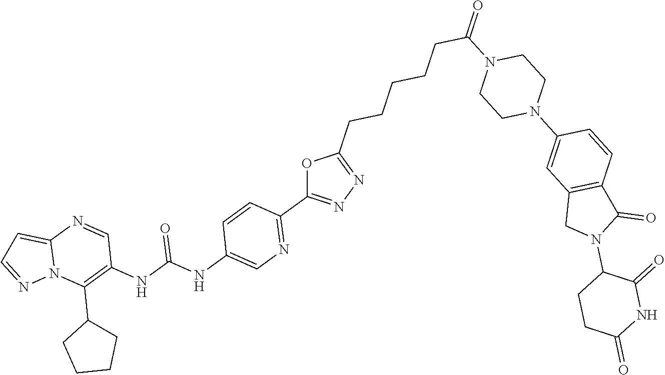 [SMILES: O=C1CCC(N2Cc3cc(N4CCN(C(=O)CCCCCc5nnc(-c6ccc(NC(=O)Nc7cnc8ccnn8c7C7CCCC7)cn6)o5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,3,4-oxadiazol-2-yl]-3- pyridyl]urea |
| 112 | 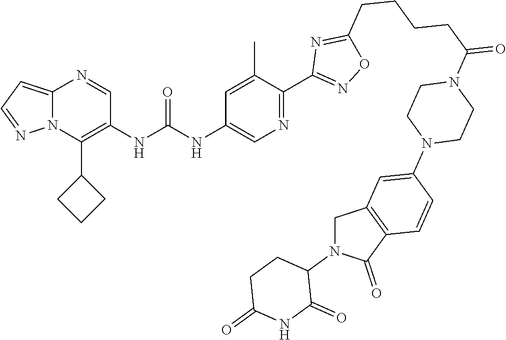 [SMILES: Cc1cc(NC(=O)Nc2cnc3ccnn3c2C2CCC2)cnc1-c1noc(CCCCC(=O)N2CCN(c3ccc4c(c3)CN(C3CCC(=O)NC3=O)C4=O)CC2)n1] 1-(7-cyclobutylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- methyl-3-pyridyl]urea |
| 113 | 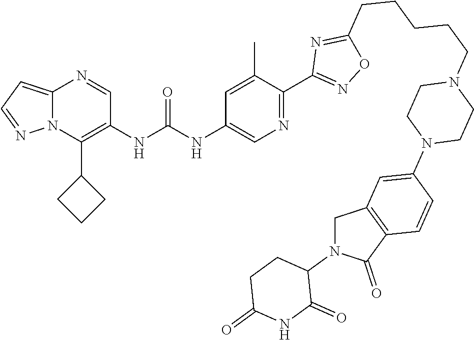 [SMILES: Cc1cc(NC(=O)Nc2cnc3ccnn3c2C2CCC2)cnc1-c1noc(CCCCCN2CCN(c3ccc4c(c3)CN(C3CCC(=O)NC3=O)C4=O)CC2)n1] 1-(7-cyclobutylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- methyl-3-pyridyl]urea |
| 114 | 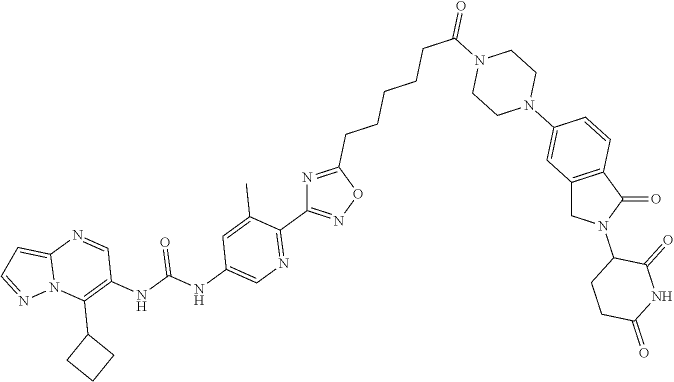 [SMILES: Cc1cc(NC(=O)Nc2cnc3ccnn3c2C2CCC2)cnc1-c1noc(CCCCCC(=O)N2CCN(c3ccc4c(c3)CN(C3CCC(=O)NC3=O)C4=O)CC2)n1] 1-(7-cyclobutylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 115 | 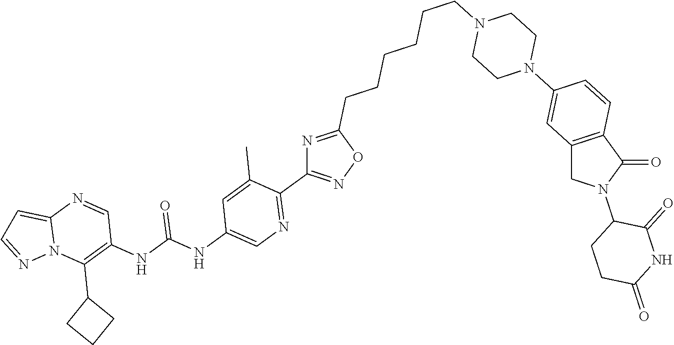 [SMILES: Cc1cc(NC(=O)Nc2cnc3ccnn3c2C2CCC2)cnc1-c1noc(CCCCCCN2CCN(c3ccc4c(c3)CN(C3CCC(=O)NC3=O)C4=O)CC2)n1] 1-(7-cyclobutylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-methyl- 3-pyridyl]urea |
| 116 | 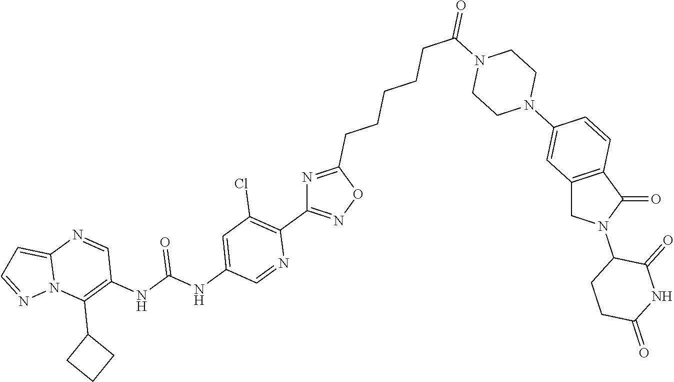 [SMILES: O=C1CCC(N2Cc3cc(N4CCN(C(=O)CCCCCc5nc(-c6ncc(NC(=O)Nc7cnc8ccnn8c7C7CCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-cyclobutylpyrazolo[1,5- a]pyrimidin-6-yl)urea |
| 117 | 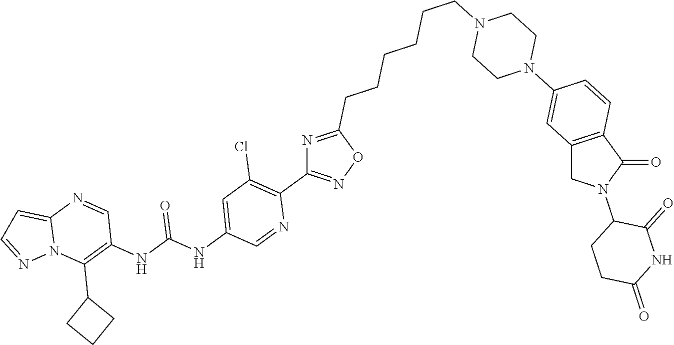 [SMILES: O=C1CCC(N2Cc3cc(N4CCN(CCCCCCc5nc(-c6ncc(NC(=O)Nc7cnc8ccnn8c7C7CCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-cyclobutylpyrazolo[1,5-a]pyrimidin-6- yl)urea |
| 118 | 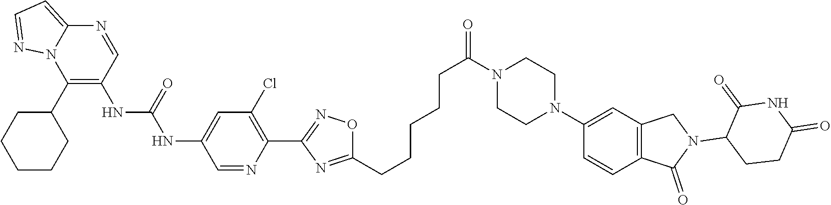 [SMILES: O=C1CCC(N2Cc3cc(N4CCN(C(=O)CCCCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-cyclohexylpyrazolo[1,5- a]pyrimidin-6-yl)urea |
| 119 | 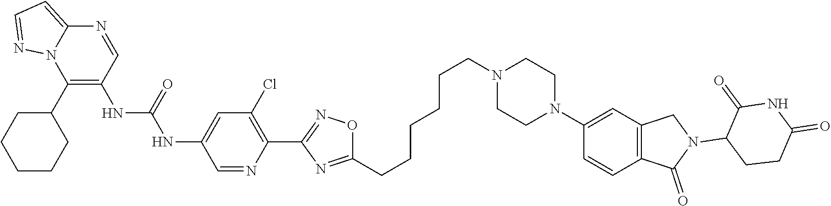 [SMILES: O=C1CCC(N2Cc3cc(N4CCN(CCCCCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-cyclohexylpyrazolo[1,5-a]pyrimidin-6- yl)urea |
| 120 | 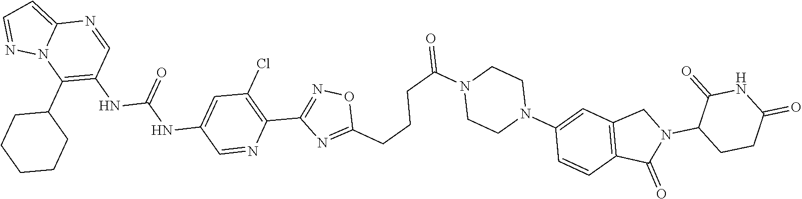 [SMILES: O=C1CCC(N2Cc3cc(N4CCN(C(=O)CCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]-4-oxo-butyl]-1,2,4-oxadiazol-3-yl]-3-pyridyl]-3-(7-cyclohexylpyrazolo[1,5- a]pyrimidin-6-yl)urea |
| 121 | 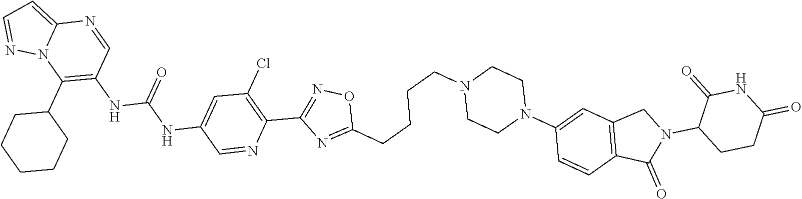 [SMILES: O=C1CCC(N2Cc3cc(N4CCN(CCCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]butyl]-1,2,4-oxadiazol-3-yl]-3-pyridyl]-3-(7-cyclohexylpyrazolo[1,5-a]pyrimidin-6- yl)urea |
| 122 | 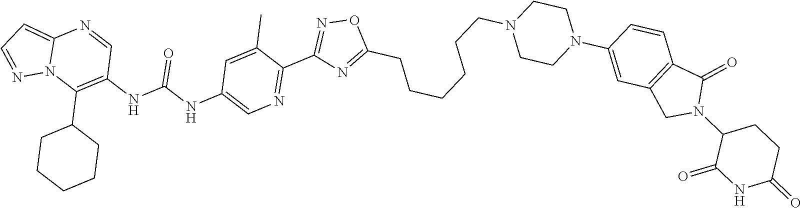 [SMILES: Cc1cc(NC(=O)Nc2cnc3ccnn3c2C2CCCCC2)cnc1-c1noc(CCCCCCN2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-methyl- 3-pyridyl]urea |
| 123 | 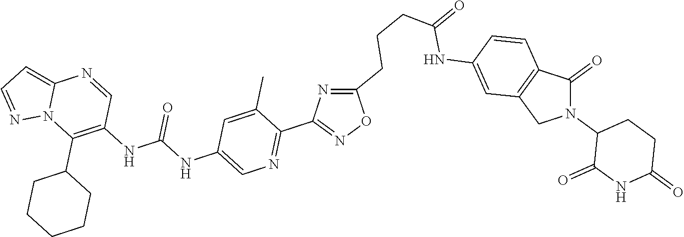 [SMILES: Cc1cc(NC(=O)Nc2cnc3ccnn3c2C2CCCCC2)cnc1-c1noc(CCCC(=O)Nc2ccc3c(c2)CN(C2CCC(=O)NC2=O)C3=O)n1] 4-[3-[5-[(7-cyclohexylpyrazolo[1,5-a]pyrimidin-6-yl)carbamoylamino]-3-methyl-2- pyridyl]-1,2,4-oxadiazol-5-yl]-N-[2-(2,6-dioxo-3-piperidyl)-1-oxo-isoindolin-5- yl]butanamide |
| 124 | 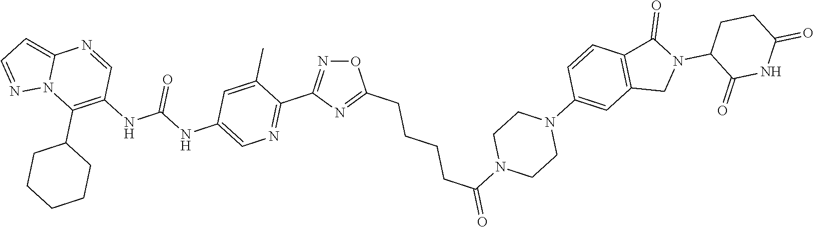 [SMILES: Cc1cc(NC(=O)Nc2cnc3ccnn3c2C2CCCCC2)cnc1-c1noc(CCCCC(=O)N2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- methyl-3-pyridyl]urea |
| 125 | 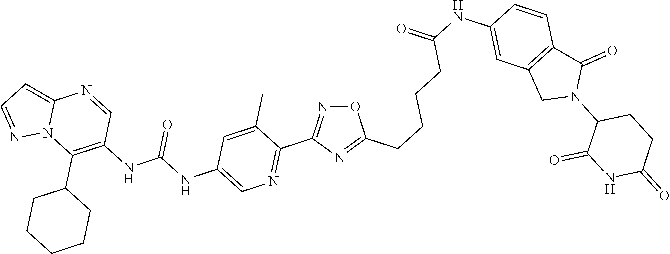 [SMILES: Cc1cc(NC(=O)Nc2cnc3ccnn3c2C2CCCCC2)cnc1-c1noc(CCCCC(=O)Nc2ccc3c(c2)CN(C2CCC(=O)NC2=O)C3=O)n1] 5-[3-[5-[(7-cyclohexylpyrazolo[1,5-a]pyrimidin-6-yl)carbamoylamino]-3-methyl-2- pyridyl]-1,2,4-oxadiazol-5-yl]-N-[2-(2,6-dioxo-3-piperidyl)-1-oxo-isoindolin-5- yl]pentanamide |
| 126 | 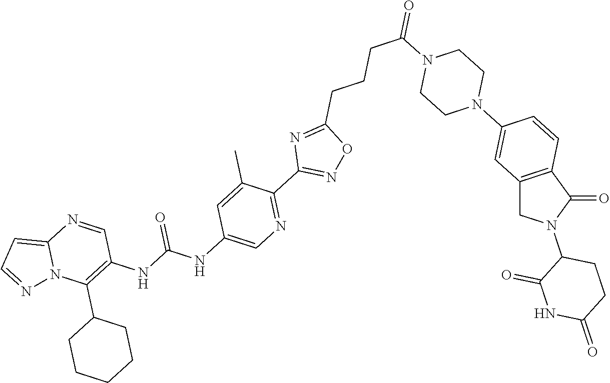 [SMILES: Cc1cc(NC(=O)Nc2cnc3ccnn3c2C2CCCCC2)cnc1-c1noc(CCCC(=O)N2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- methyl-3-pyridyl]urea |
| 127 | 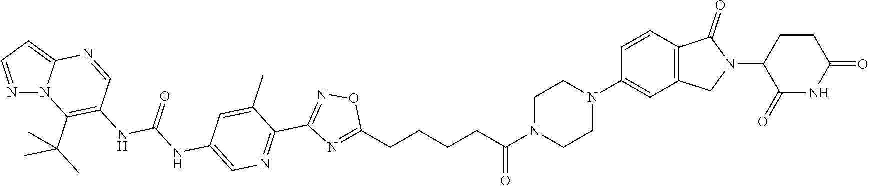 [SMILES: Cc1cc(NC(=O)Nc2cnc3ccnn3c2C(C)(C)C)cnc1-c1noc(CCCCC(=O)N2CCN(c3ccc4c(c3)CN(C3CCC(=O)NC3=O)C4=O)CC2)n1] 1-(7-tert-butylpyrazolo[1,5-a]pyrimidin-6-yl)-3-[6-[5-[5-[4-[2-(2,6-dioxo-3-piperidyl)- 1-oxo-isoindolin-5-yl]piperazin-1-yl]-5-oxo-pentyl]-1,2,4-oxadiazol-3-yl]-5-methyl-3- pyridyl]urea |
| 128 | 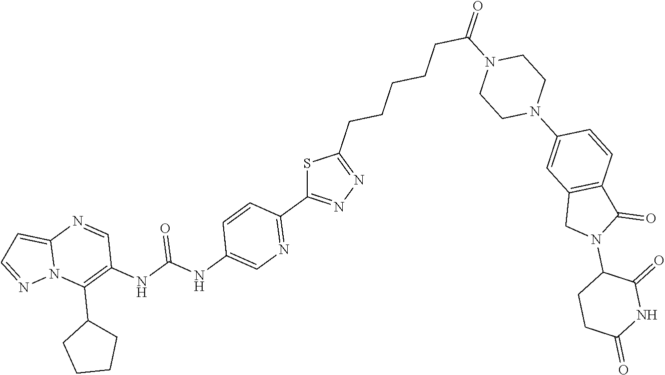 [SMILES: O=C1CCC(N2Cc3cc(N4CCN(C(=O)CCCCCc5nnc(-c6ccc(NC(=O)Nc7cnc8ccnn8c7C7CCCC7)cn6)s5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,3,4-thiadiazol-2-yl]-3- pyridyl]urea |
| 129 | 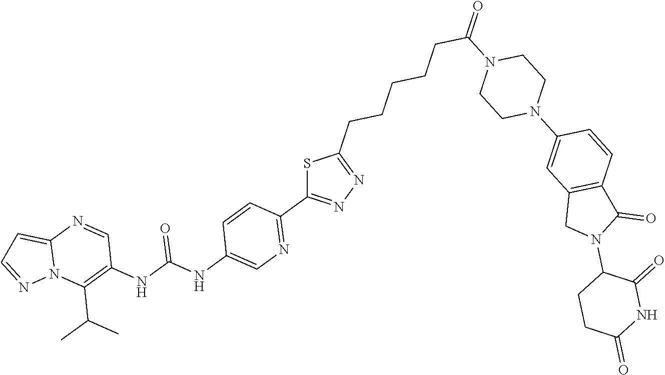 [SMILES: CC(C)c1c(NC(=O)Nc2ccc(-c3nnc(CCCCCC(=O)N4CCN(c5ccc6c(c5)CN(C5CCC(=O)NC5=O)C6=O)CC4)s3)nc2)cnc2ccnn12] 1-[6-[5-[6-[4-[2-(2,6-dioxo-3-piperidyl)-1-oxo-isoindolin-5-yl]piperazin-1-yl]-6-oxo- hexyl]-1,3,4-thiadiazol-2-yl]-3-pyridyl]-3-(7-isopropylpyrazolo[1,5-a]pyrimidin-6- yl)urea |
| 130 | 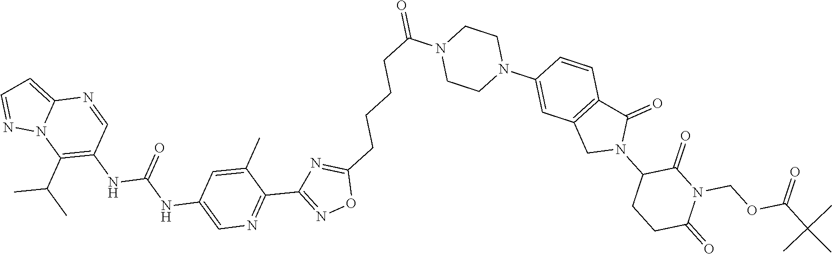 [SMILES: Cc1cc(NC(=O)Nc2cnc3ccnn3c2C(C)C)cnc1-c1noc(CCCCC(=O)N2CCN(c3ccc4c(c3)CN(C3CCC(=O)N(COC(=O)C(C)(C)C)C3=O)C4=O)CC2)n1] [3-[5-[4-[5-[3-[5-[(7-isopropylpyrazolo[1,5-a]pyrimidin-6-yl)carbamoylamino]-3- methyl-2-pyridyl]-1,2,4-oxadiazol-5-yl]pentanoyl]piperazin-1-yl]-1-oxo-isoindolin-2- yl]-2,6-dioxo-1-piperidyl]methyl 2,2-dimethylpropanoate |
| 131 | 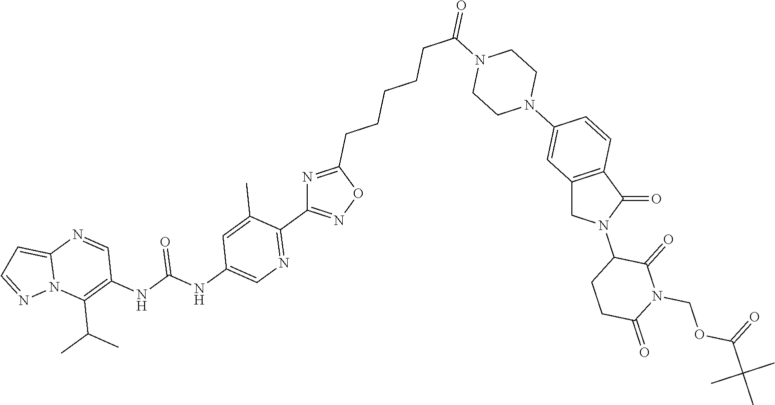 [SMILES: Cc1cc(NC(=O)Nc2cnc3ccnn3c2C(C)C)cnc1-c1noc(CCCCCC(=O)N2CCN(c3ccc4c(c3)CN(C3CCC(=O)N(COC(=O)C(C)(C)C)C3=O)C4=O)CC2)n1] [3-[5-[4-[6-[3-[5-[(7-isopropylpyrazolo[1,5-a]pyrimidin-6-yl)carbamoylamino]-3- methyl-2-pyridyl]-1,2,4-oxadiazol-5-yl]hexanoyl]piperazin-1-yl]-1-oxo-isoindolin-2- yl]-2,6-dioxo-1-piperidyl]methyl 2,2-dimethylpropanoate |
| 132 | 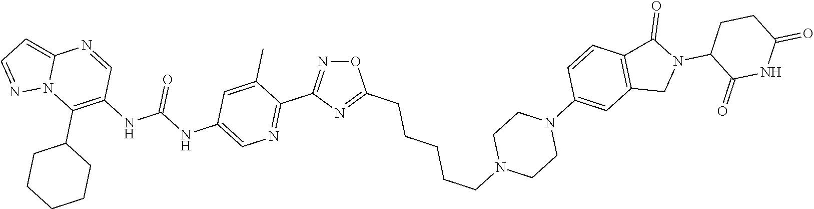 [SMILES: Cc1cc(NC(=O)Nc2cnc3ccnn3c2C2CCCCC2)cnc1-c1noc(CCCCCN2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- methyl-3-pyridyl]urea |
| 133 | 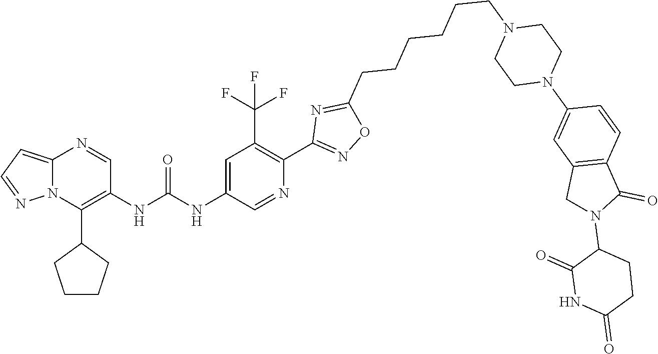 [SMILES: O=C1CCC(N2Cc3cc(N4CCN(CCCCCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5- (trifluoromethyl)-3-pyridyl]urea |
| 134 | 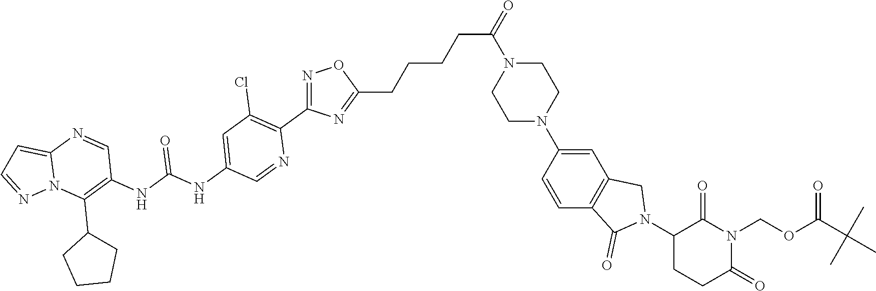 [SMILES: CC(C)(C)C(=O)OCN1C(=O)CCC(N2Cc3cc(N4CCN(C(=O)CCCCc5nc(-c6ncc(NC(=O)Nc7cnc8ccnn8c7C7CCCC7)cc6Cl)no5)CC4)ccc3C2=O)C1=O] [3-[5-[4-[5-[3-[3-chloro-5-[(7-cyclopentylpyrazolo[1,5-a]pyrimidin-6- yl)carbamoylamino]-2-pyridyl]-1,2,4-oxadiazol-5-yl]pentanoyl]piperazin-1-yl]-1-oxo- isoindolin-2-yl]-2,6-dioxo-1-piperidyl]methyl 2,2-dimethylpropanoate |
| 135 | 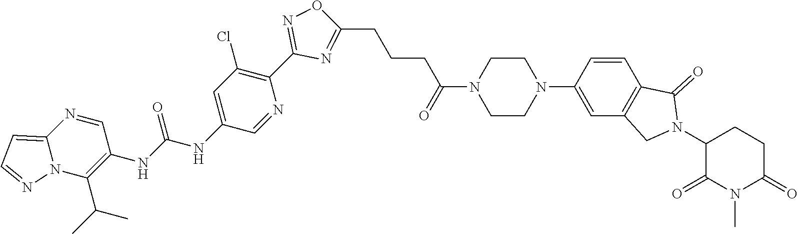 [SMILES: CC(C)c1c(NC(=O)Nc2cnc(-c3noc(CCCC(=O)N4CCN(c5ccc6c(c5)CN(C5CCC(=O)N(C)C5=O)C6=O)CC4)n3)c(Cl)c2)cnc2ccnn12] 1-[5-chloro-6-[5-[4-[4-[2-(1-methyl-2,6-dioxo-3-piperidyl)-1-oxo-isoindolin-5- yl]piperazin-1-yl]-4-oxo-butyl]-1,2,4-oxadiazol-3-yl]-3-pyridyl]-3-(7- isopropylpyrazolo[1,5-a]pyrimidin-6-yl)urea |
| 136 | 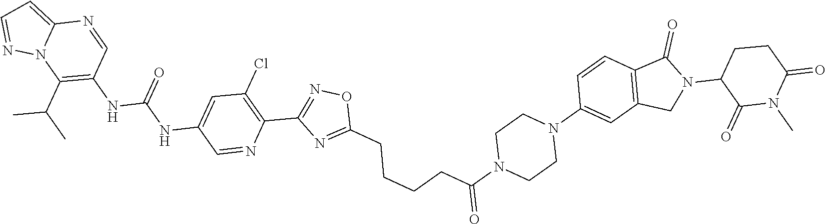 [SMILES: CC(C)c1c(NC(=O)Nc2cnc(-c3noc(CCCCC(=O)N4CCN(c5ccc6c(c5)CN(C5CCC(=O)N(C)C5=O)C6=O)CC4)n3)c(Cl)c2)cnc2ccnn12] 1-[5-chloro-6-[5-[5-[4-[2-(1-methyl-2,6-dioxo-3-piperidyl)-1-oxo-isoindolin-5- yl]piperazin-1-yl]-5-oxo-pentyl]-1,2,4-oxadiazol-3-yl]-3-pyridyl]-3-(7- isopropylpyrazolo[1,5-a]pyrimidin-6-yl)urea |
| 137 | 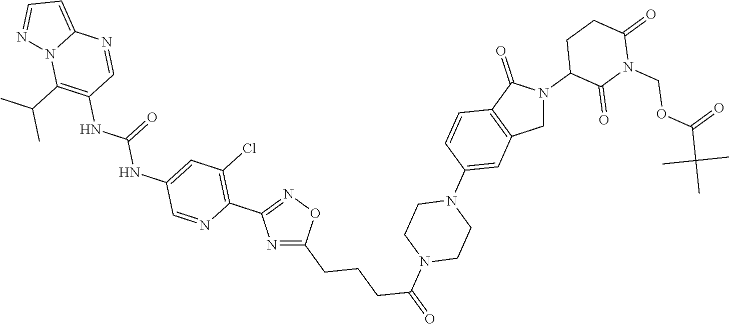 [SMILES: CC(C)c1c(NC(=O)Nc2cnc(-c3noc(CCCC(=O)N4CCN(c5ccc6c(c5)CN(C5CCC(=O)N(COC(=O)C(C)(C)C)C5=O)C6=O)CC4)n3)c(Cl)c2)cnc2ccnn12] [3-[5-[4-[4-[3-[3-chloro-5-[(7-isopropylpyrazolo[1,5-a]pyrimidin-6- yl)carbamoylamino]-2-pyridyl]-1,2,4-oxadiazol-5-yl]butanoyl]piperazin-1-yl]-1-oxo- isoindolin-2-yl]-2,6-dioxo-1-piperidyl]methyl 2,2-dimethylpropanoate |
| 138 | 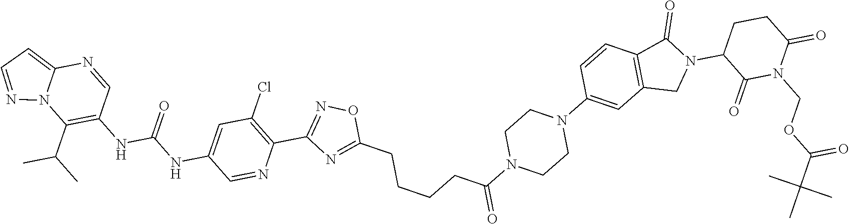 [SMILES: CC(C)c1c(NC(=O)Nc2cnc(-c3noc(CCCCC(=O)N4CCN(c5ccc6c(c5)CN(C5CCC(=O)N(COC(=O)C(C)(C)C)C5=O)C6=O)CC4)n3)c(Cl)c2)cnc2ccnn12] [3-[5-[4-[5-[3-[3-chloro-5-[(7-isopropylpyrazolo[1,5-a]pyrimidin-6- yl)carbamoylamino]-2-pyridyl]-1,2,4-oxadiazol-5-yl]pentanoyl]piperazin-1-yl]-1-oxo- isoindolin-2-yl]-2,6-dioxo-1-piperidyl]methyl 2,2-dimethylpropanoate |
| 139 | 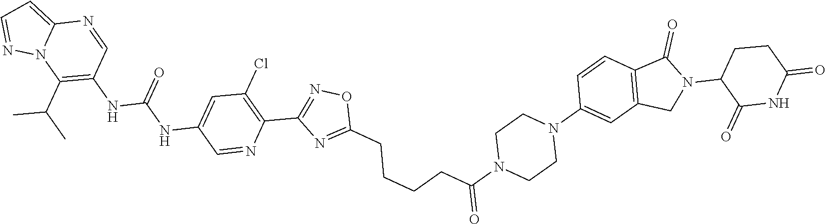 [SMILES: CC(C)c1c(NC(=O)Nc2cnc(-c3noc(CCCCC(=O)N4CCN(c5ccc6c(c5)CN(C5CCC(=O)NC5=O)C6=O)CC4)n3)c(Cl)c2)cnc2ccnn12] 1-[5-chloro-6-[5-[5-[4-[2-(2,6-dioxo-3-piperidyl)-1-oxo-isoindolin-5-yl]piperazin-1- yl]-5-oxo-pentyl]-1,2,4-oxadiazol-3-yl]-3-pyridyl]-3-(7-isopropylpyrazolo[1,5- a]pyrimidin-6-yl)urea |
| 140 | 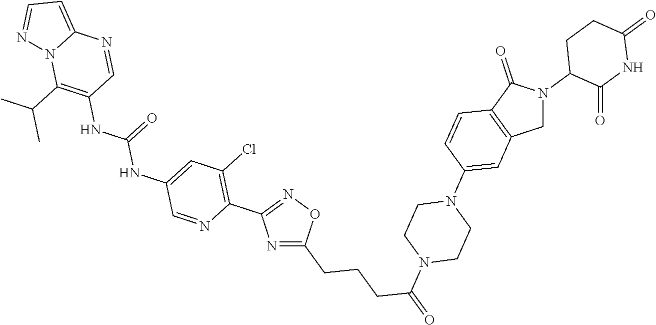 [SMILES: CC(C)c1c(NC(=O)Nc2cnc(-c3noc(CCCC(=O)N4CCN(c5ccc6c(c5)CN(C5CCC(=O)NC5=O)C6=O)CC4)n3)c(Cl)c2)cnc2ccnn12] 1-[5-chloro-6-[5-[4-[4-[2-(2,6-dioxo-3-piperidyl)-1-oxo-isoindolin-5-yl]piperazin-1- yl]-4-oxo-butyl]-1,2,4-oxadiazol-3-yl]-3-pyridyl]-3-(7-isopropylpyrazolo[1,5- a]pyrimidin-6-yl)urea |
| 141 | 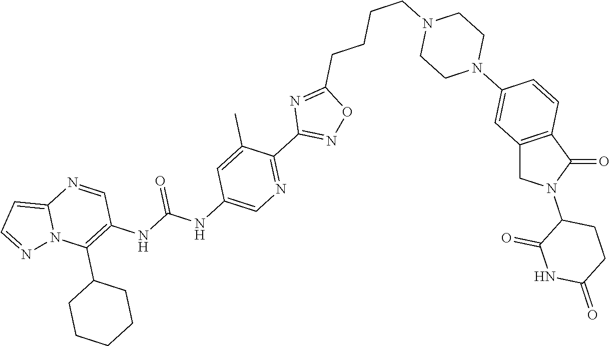 [SMILES: Cc1cc(NC(=O)Nc2cnc3ccnn3c2C2CCCCC2)cnc1-c1noc(CCCCN2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butyl]-1,2,4-oxadiazol-3-yl]-5-methyl- 3-pyridyl]urea |
| 142 | 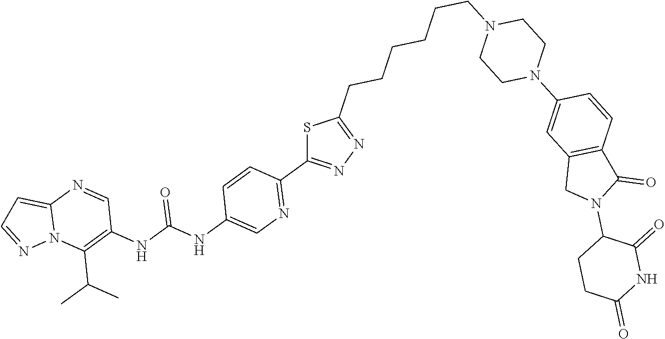 [SMILES: CC(C)c1c(NC(=O)Nc2ccc(-c3nnc(CCCCCCN4CCN(c5ccc6c(c5)CN(C5CCC(=O)NC5=O)C6=O)CC4)s3)nc2)cnc2ccnn12] 1-[6-[5-[6-[4-[2-(2,6-dioxo-3-piperidyl)-1-oxo-isoindolin-5-yl]piperazin-1-yl]hexyl]- 1,3,4-thiadiazol-2-yl]-3-pyridyl]-3-(7-isopropylpyrazolo[1,5-a]pyrimidin-6-yl)urea |
| 143 | 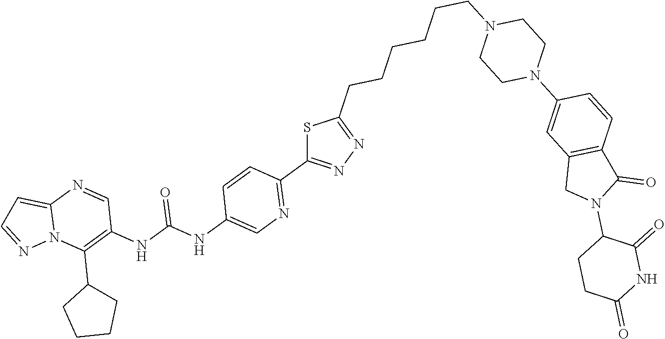 [SMILES: O=C1CCC(N2Cc3cc(N4CCN(CCCCCCc5nnc(-c6ccc(NC(=O)Nc7cnc8ccnn8c7C7CCCC7)cn6)s5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,3,4-thiadiazol-2-yl]-3- pyridyl]urea |
| 144 | 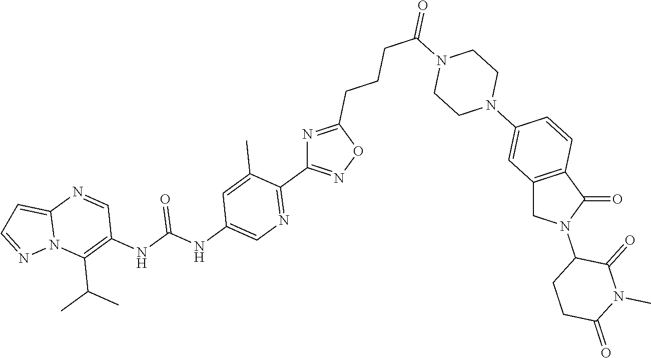 [SMILES: Cc1cc(NC(=O)Nc2cnc3ccnn3c2C(C)C)cnc1-c1noc(CCCC(=O)N2CCN(c3ccc4c(c3)CN(C3CCC(=O)N(C)C3=O)C4=O)CC2)n1] 1-(7-isopropylpyrazolo[1,5-a]pyrimidin-6-yl)-3-[5-methyl-6-[5-[4-[4-[2-(1-methyl-2,6- dioxo-3-piperidyl)-1-oxo-isoindolin-5-yl]piperazin-1-yl]-4-oxo-butyl]-1,2,4-oxadiazol- 3-yl]-3-pyridyl]urea |
| 145 | 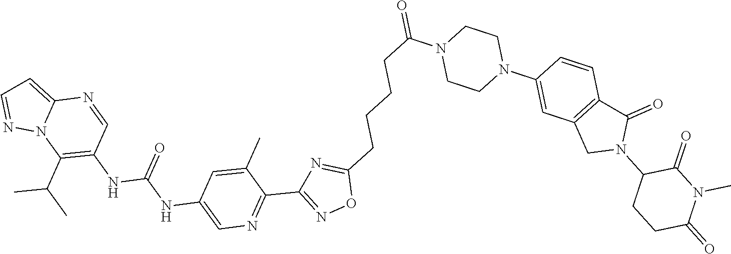 [SMILES: Cc1cc(NC(=O)Nc2cnc3ccnn3c2C(C)C)cnc1-c1noc(CCCCC(=O)N2CCN(c3ccc4c(c3)CN(C3CCC(=O)N(C)C3=O)C4=O)CC2)n1] 1-(7-isopropylpyrazolo[1,5-a]pyrimidin-6-yl)-3-[5-methyl-6-[5-[5-[4-[2-(1-methyl-2,6- dioxo-3-piperidyl)-1-oxo-isoindolin-5-yl]piperazin-1-yl]-5-oxo-pentyl]-1,2,4- oxadiazol-3-yl]-3-pyridyl]urea |
| 146 | 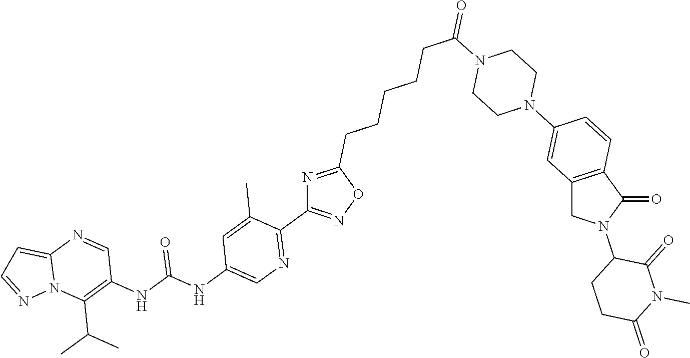 [SMILES: Cc1cc(NC(=O)Nc2cnc3ccnn3c2C(C)C)cnc1-c1noc(CCCCCC(=O)N2CCN(c3ccc4c(c3)CN(C3CCC(=O)N(C)C3=O)C4=O)CC2)n1] 1-(7-isopropylpyrazolo[1,5-a]pyrimidin-6-yl)-3-[5-methyl-6-[5-[6-[4-[2-(1-methyl-2,6- dioxo-3-piperidyl)-1-oxo-isoindolin-5-yl]piperazin-1-yl]-6-oxo-hexyl]-1,2,4- oxadiazol-3-yl]-3-pyridyl]urea |
| 147 | 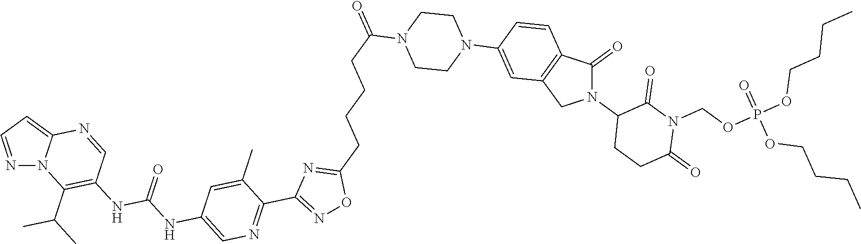 [SMILES: CCCCOP(=O)(OCCCC)OCN1C(=O)CCC(N2Cc3cc(N4CCN(C(=O)CCCCc5nc(-c6ncc(NC(=O)Nc7cnc8ccnn8c7C(C)C)cc6C)no5)CC4)ccc3C2=O)C1=O] dibutyl [3-[5-[4-[5-[3-[5-[(7-isopropylpyrazolo[1,5-a]pyrimidin-6- yl)carbamoylamino]-3-methyl-2-pyridyl]-1,2,4-oxadiazol-5-yl]pentanoyl]piperazin-1- yl]-1-oxo-isoindolin-2-yl]-2,6-dioxo-1-piperidyl]methyl phosphate |
| 148 | 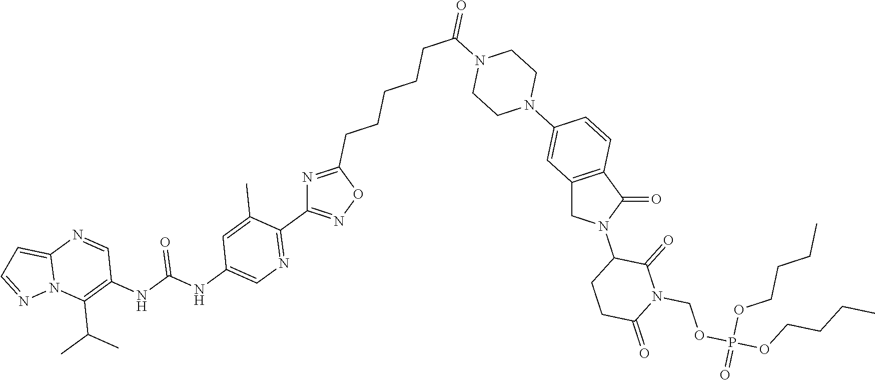 [SMILES: CCCCOP(=O)(OCCCC)OCN1C(=O)CCC(N2Cc3cc(N4CCN(C(=O)CCCCCc5nc(-c6ncc(NC(=O)Nc7cnc8ccnn8c7C(C)C)cc6C)no5)CC4)ccc3C2=O)C1=O] dibutyl [3-[5-[4-[6-[3-[5-[(7-isopropylpyrazolo[1,5-a]pyrimidin-6- yl)carbamoylamino]-3-methyl-2-pyridyl]-1,2,4-oxadiazol-5-yl]hexanoyl]piperazin-1- yl]-1-oxo-isoindolin-2-yl]-2,6-dioxo-1-piperidyl]methyl phosphate |
| 149 | 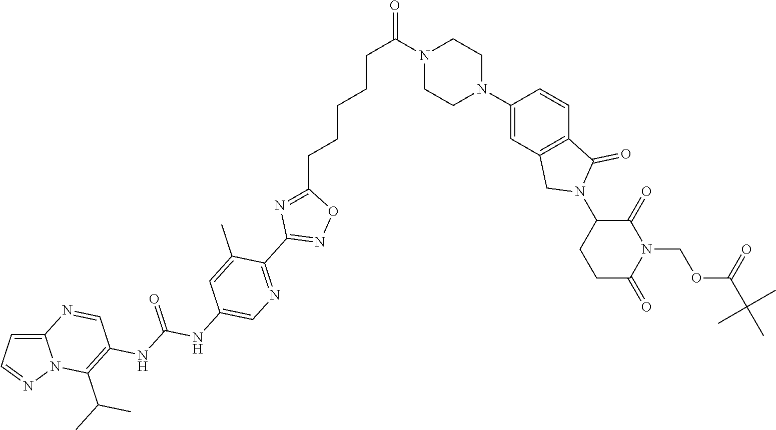 [SMILES: Cc1cc(NC(=O)Nc2cnc3ccnn3c2C(C)C)cnc1-c1noc(CCCCCC(=O)N2CCN(c3ccc4c(c3)CN(C3CCC(=O)N(COC(=O)C(C)(C)C)C3=O)C4=O)CC2)n1] [3-[5-[4-[4-[3-[5-[(7-isopropylpyrazolo[1,5-a]pyrimidin-6-yl)carbamoylamino]-3- methyl-2-pyridyl]-1,2,4-oxadiazol-5-yl]butanoyl]piperazin-1-yl]-1-oxo-isoindolin-2- yl]-2,6-dioxo-1-piperidyl]methyl 2,2-dimethylpropanoate |
| 150 | 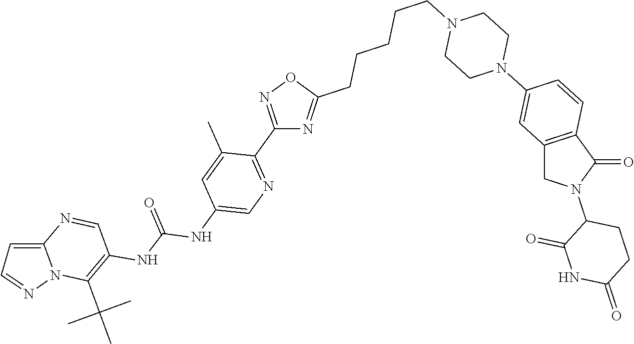 [SMILES: Cc1cc(NC(=O)Nc2cnc3ccnn3c2C(C)(C)C)cnc1-c1noc(CCCCCN2CCN(c3ccc4c(c3)CN(C3CCC(=O)NC3=O)C4=O)CC2)n1] 1-(7-tert-butylpyrazolo[1,5-a]pyrimidin-6-yl)-3-[6-[5-[5-[4-[2-(2,6-dioxo-3-piperidyl)- 1-oxo-isoindolin-5-yl]piperazin-1-yl]pentyl]-1,2,4-oxadiazol-3-yl]-5-methyl-3- pyridyl]urea |
| 151 | 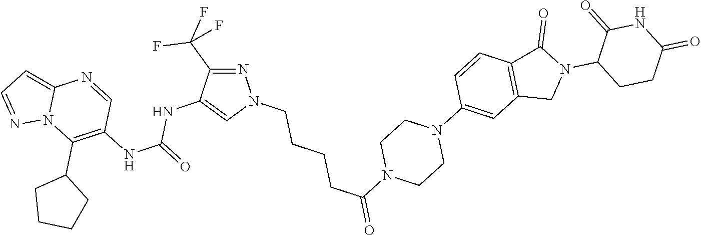 [SMILES: O=C1CCC(N2Cc3cc(N4CCN(C(=O)CCCCn5cc(NC(=O)Nc6cnc7ccnn7c6C6CCCC6)c(C(F)(F)F)n5)CC4)ccc3C2=O)C(=O)N1] 1-(7-cyclopentylpyrazolo[1,5-a]pyrimidin-6-yl)-3-[1-[5-[4-[2-(2,6-dioxo-3-piperidyl)- 1-oxo-isoindolin-5-yl]piperazin-1-yl]-5-oxo-pentyl]-3-(trifluoromethyl)pyrazol-4- yl]urea |
| 152 | 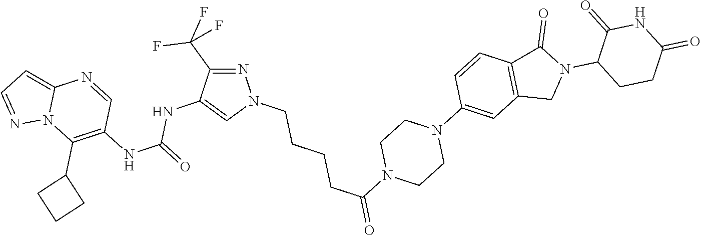 [SMILES: O=C1CCC(N2Cc3cc(N4CCN(C(=O)CCCCn5cc(NC(=O)Nc6cnc7ccnn7c6C6CCC6)c(C(F)(F)F)n5)CC4)ccc3C2=O)C(=O)N1] 1-(7-cyclobutylpyrazolo[1,5-a]pyrimidin-6-yl)-3-[1-[5-[4-[2-(2,6-dioxo-3-piperidyl)-1- oxo-isoindolin-5-yl]piperazin-1-yl]-5-oxo-pentyl]-3-(trifluoromethyl)pyrazol-4-yl]urea |
| 153 | 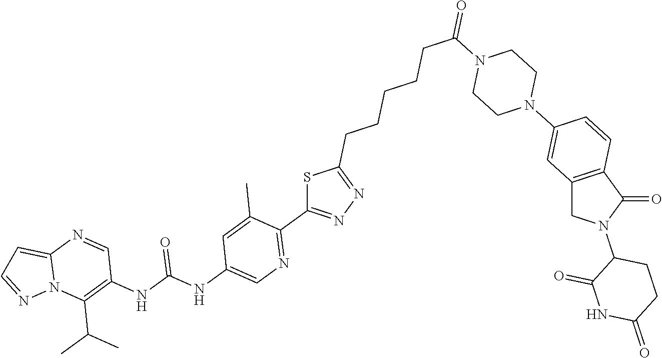 [SMILES: Cc1cc(NC(=O)Nc2cnc3ccnn3c2C(C)C)cnc1-c1nnc(CCCCCC(=O)N2CCN(c3ccc4c(c3)CN(C3CCC(=O)NC3=O)C4=O)CC2)s1] 1-[6-[5-[6-[4-[2-(2,6-dioxo-3-piperidyl)-1-oxo-isoindolin-5-yl]piperazin-1-yl]-6-oxo- hexyl]-1,3,4-thiadiazol-2-yl]-5-methyl-3-pyridyl]-3-(7-isopropylpyrazolo[1,5- a]pyrimidin-6-yl)urea |
| 154 | 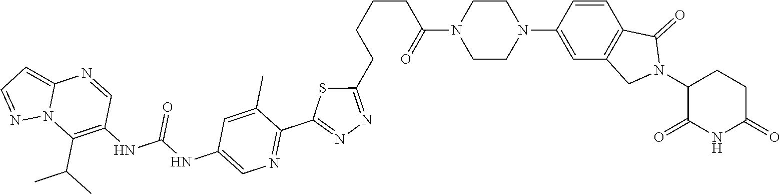 [SMILES: Cc1cc(NC(=O)Nc2cnc3ccnn3c2C(C)C)cnc1-c1nnc(CCCCC(=O)N2CCN(c3ccc4c(c3)CN(C3CCC(=O)NC3=O)C4=O)CC2)s1] 1-[6-[5-[5-[4-[2-(2,6-dioxo-3-piperidyl)-1-oxo-isoindolin-5-yl]piperazin-1-yl]-5-oxo- pentyl]-1,3,4-thiadiazol-2-yl]-5-methyl-3-pyridyl]-3-(7-isopropylpyrazolo[1,5- a]pyrimidin-6-yl)urea |
| 155 | 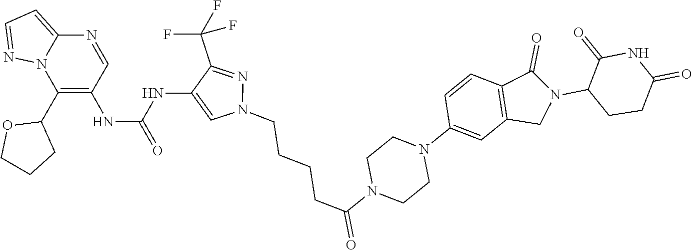 [SMILES: O=C1CCC(N2Cc3cc(N4CCN(C(=O)CCCCn5cc(NC(=O)Nc6cnc7ccnn7c6C6CCCO6)c(C(F)(F)F)n5)CC4)ccc3C2=O)C(=O)N1] 1-[1-[5-[4-[2-(2,6-dioxo-3-piperidyl)-1-oxo-isoindolin-5-yl]piperazin-1-yl]-5-oxo- pentyl]-3-(trifluoromethyl)pyrazol-4-yl]-3-(7-tetrahydrofuran-2-ylpyrazolo[1,5- a]pyrimidin-6-yl)urea |
| 156 | 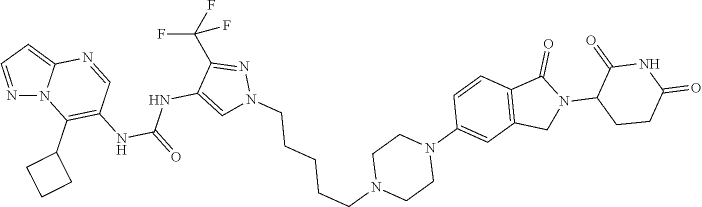 [SMILES: O=C1CCC(N2Cc3cc(N4CCN(CCCCCn5cc(NC(=O)Nc6cnc7ccnn7c6C6CCC6)c(C(F)(F)F)n5)CC4)ccc3C2=O)C(=O)N1] 1-(7-cyclobutylpyrazolo[1,5-a]pyrimidin-6-yl)-3-[1-[5-[4-[2-(2,6-dioxo-3-piperidyl)-1- oxo-isoindolin-5-yl]piperazin-1-yl]pentyl]-3-(trifluoromethyl)pyrazol-4-yl]urea |
| 157 | 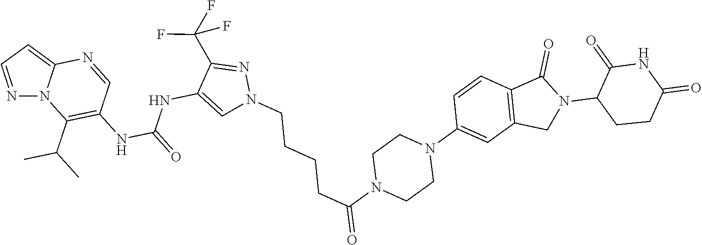 [SMILES: CC(C)c1c(NC(=O)Nc2cn(CCCCC(=O)N3CCN(c4ccc5c(c4)CN(C4CCC(=O)NC4=O)C5=O)CC3)nc2C(F)(F)F)cnc2ccnn12] 1-[1-[5-[4-[2-(2,6-dioxo-3-piperidyl)-1-oxo-isoindolin-5-yl]piperazin-1-yl]-5-oxo- pentyl]-3-(trifluoromethyl)pyrazol-4-yl]-3-(7-isopropylpyrazolo[1,5-a]pyrimidin-6- yl)urea |
| 158 | 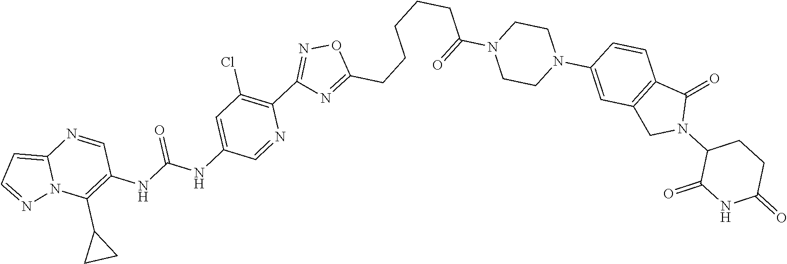 [SMILES: O=C1CCC(N2Cc3cc(N4CCN(C(=O)CCCCCc5nc(-c6ncc(NC(=O)Nc7cnc8ccnn8c7C7CC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-cyclopropylpyrazolo[1,5- a]pyrimidin-6-yl)urea |
| 159 | 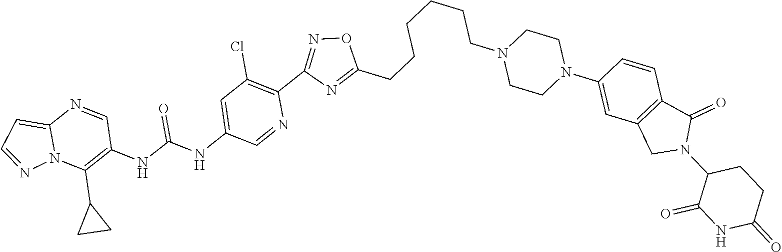 [SMILES: O=C1CCC(N2Cc3cc(N4CCN(CCCCCCc5nc(-c6ncc(NC(=O)Nc7cnc8ccnn8c7C7CC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-cyclopropylpyrazolo[1,5-a]pyrimidin- 6-yl)urea |
| 160 | 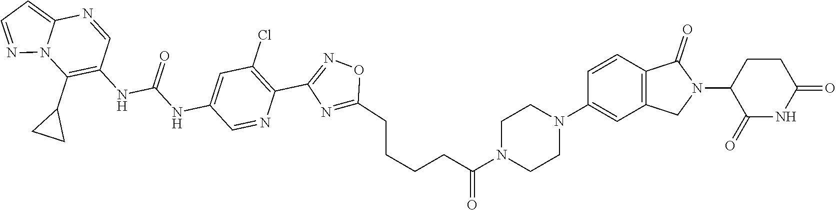 [SMILES: O=C1CCC(N2Cc3cc(N4CCN(C(=O)CCCCc5nc(-c6ncc(NC(=O)Nc7cnc8ccnn8c7C7CC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]-5-oxo-pentyl]-1,2,4-oxadiazol-3-yl]-3-pyridyl]-3-(7-cyclopropylpyrazolo[1,5- a]pyrimidin-6-yl)urea |
| 161 | 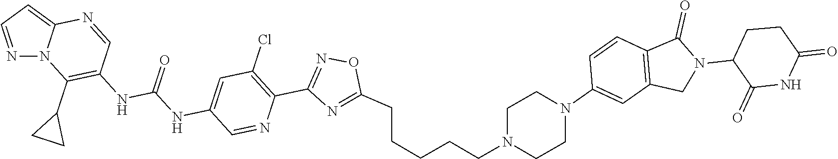 [SMILES: O=C1CCC(N2Cc3cc(N4CCN(CCCCCc5nc(-c6ncc(NC(=O)Nc7cnc8ccnn8c7C7CC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]pentyl]-1,2,4-oxadiazol-3-yl]-3-pyridyl]-3-(7-cyclopropylpyrazolo[1,5-a]pyrimidin- 6-yl)urea |
| 162 | 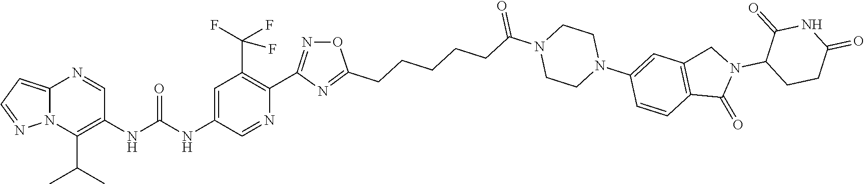 [SMILES: CC(C)c1c(NC(=O)Nc2cnc(-c3noc(CCCCCC(=O)N4CCN(c5ccc6c(c5)CN(C5CCC(=O)NC5=O)C6=O)CC4)n3)c(C(F)(F)F)c2)cnc2ccnn12] 1-[6-[5-[6-[4-[2-(2,6-dioxo-3-piperidyl)-1-oxo-isoindolin-5-yl]piperazin-1-yl]-6-oxo- hexyl]-1,2,4-oxadiazol-3-yl]-5-(trifluoromethyl)-3-pyridyl]-3-(7- isopropylpyrazolo[1,5-a]pyrimidin-6-yl)urea |
| 163 | 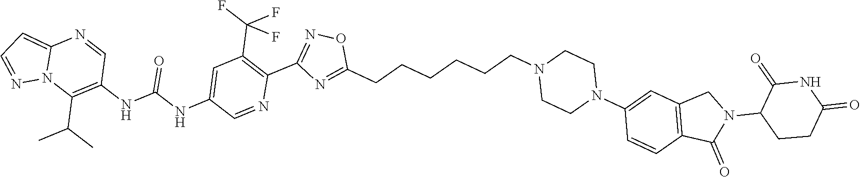 [SMILES: CC(C)c1c(NC(=O)Nc2cnc(-c3noc(CCCCCCN4CCN(c5ccc6c(c5)CN(C5CCC(=O)NC5=O)C6=O)CC4)n3)c(C(F)(F)F)c2)cnc2ccnn12] 1-[6-[5-[6-[4-[2-(2,6-dioxo-3-piperidyl)-1-oxo-isoindolin-5-yl]piperazin-1-yl]hexyl]- 1,2,4-oxadiazol-3-yl]-5-(trifluoromethyl)-3-pyridyl]-3-(7-isopropylpyrazolo[1,5- a]pyrimidin-6-yl)urea |
| 164 | 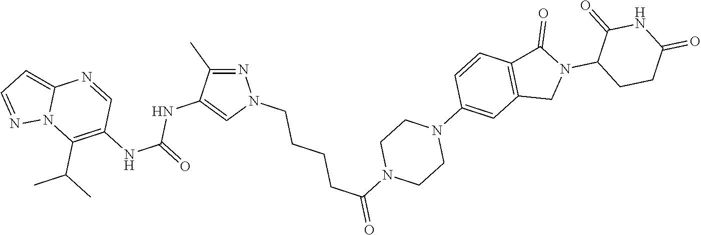 [SMILES: Cc1nn(CCCCC(=O)N2CCN(c3ccc4c(c3)CN(C3CCC(=O)NC3=O)C4=O)CC2)cc1NC(=O)Nc1cnc2ccnn2c1C(C)C] 1-[1-[5-[4-[2-(2,6-dioxo-3-piperidyl)-1-oxo-isoindolin-5-yl]piperazin-1-yl]-5-oxo- pentyl]-3-methyl-pyrazol-4-yl]-3-(7-isopropylpyrazolo[1,5-a]pyrimidin-6-yl)urea |
| 165 | 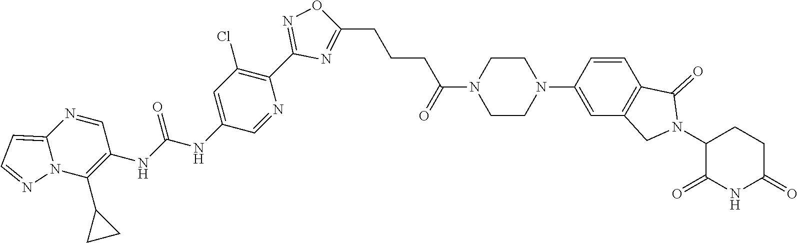 [SMILES: O=C1CCC(N2Cc3cc(N4CCN(C(=O)CCCc5nc(-c6ncc(NC(=O)Nc7cnc8ccnn8c7C7CC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]-4-oxo-butyl]-1,2,4-oxadiazol-3-yl]-3-pyridyl]-3-(7-cyclopropylpyrazolo[1,5- a]pyrimidin-6-yl)urea |
| 166 | 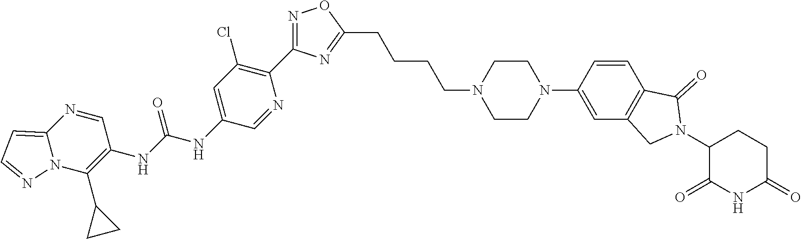 [SMILES: O=C1CCC(N2Cc3cc(N4CCN(CCCCc5nc(-c6ncc(NC(=O)Nc7cnc8ccnn8c7C7CC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]butyl]-1,2,4-oxadiazol-3-yl]-3-pyridyl]-3-(7-cyclopropylpyrazolo[1,5-a]pyrimidin- 6-yl)urea |
| 167 | 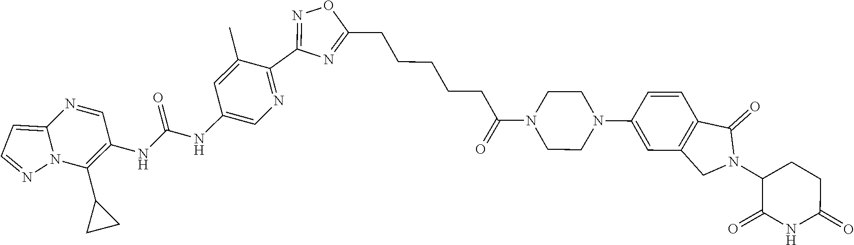 [SMILES: Cc1cc(NC(=O)Nc2cnc3ccnn3c2C2CC2)cnc1-c1noc(CCCCCC(=O)N2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 168 | 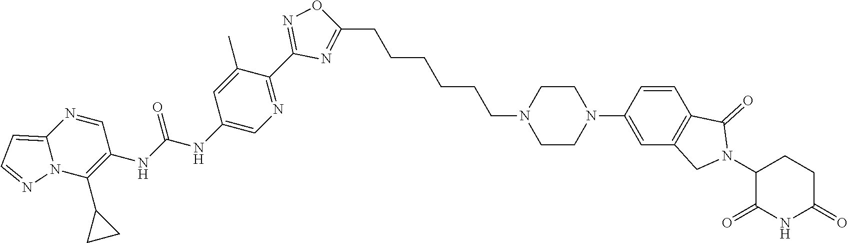 [SMILES: Cc1cc(NC(=O)Nc2cnc3ccnn3c2C2CC2)cnc1-c1noc(CCCCCCN2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-methyl- 3-pyridyl]urea |
| 169 | 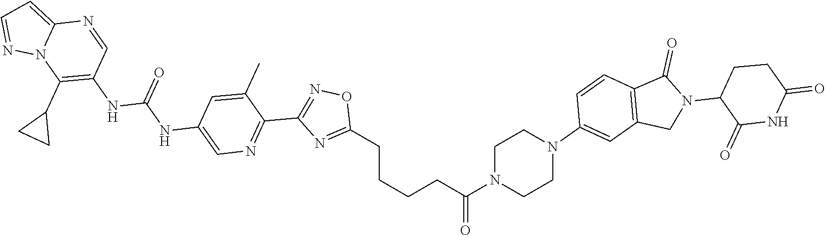 [SMILES: Cc1cc(NC(=O)Nc2cnc3ccnn3c2C2CC2)cnc1-c1noc(CCCCC(=O)N2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- methyl-3-pyridyl]urea |
| 170 | 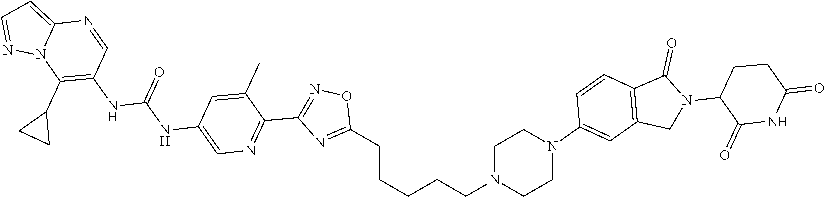 [SMILES: Cc1cc(NC(=O)Nc2cnc3ccnn3c2C2CC2)cnc1-c1noc(CCCCCN2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- methyl-3-pyridyl]urea |
| 171 | 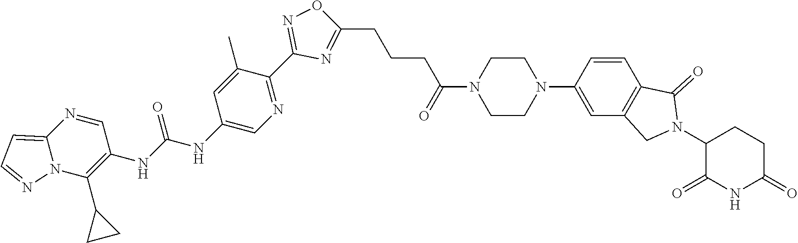 [SMILES: Cc1cc(NC(=O)Nc2cnc3ccnn3c2C2CC2)cnc1-c1noc(CCCC(=O)N2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- methyl-3-pyridyl]urea |
| 172 | 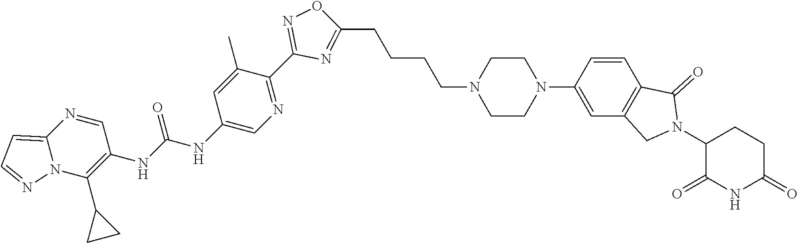 [SMILES: Cc1cc(NC(=O)Nc2cnc3ccnn3c2C2CC2)cnc1-c1noc(CCCCN2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butyl]-1,2,4-oxadiazol-3-yl]-5-methyl- 3-pyridyl]urea |
| 173 | 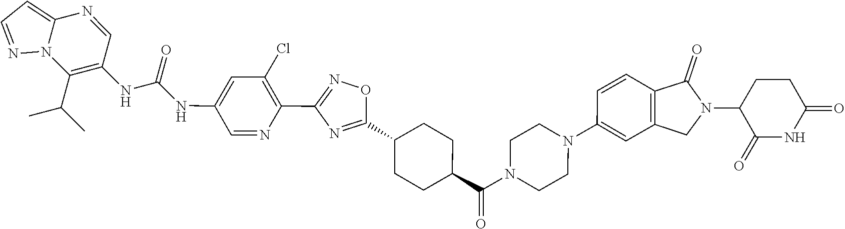 [SMILES: CC(C)c1c(NC(=O)Nc2cnc(-c3noc([C@H]4CC[C@H](C(=O)N5CCN(c6ccc7c(c6)CN(C6CCC(=O)NC6=O)C7=O)CC5)CC4)n3)c(Cl)c2)cnc2ccnn12] 1-[5-chloro-6-[5-[4-[4-[2-(2,6-dioxo-3-piperidyl)-1-oxo-isoindolin-5-yl]piperazine-1- carbonyl]cyclohexyl]-1,2,4-oxadiazol-3-yl]-3-pyridyl]-3-(7-isopropylpyrazolo[1,5- a]pyrimidin-6-yl)urea |
| 174 | 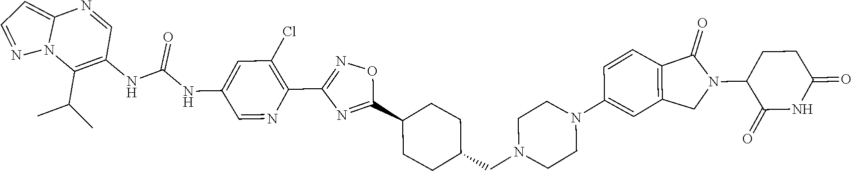 [SMILES: CC(C)c1c(NC(=O)Nc2cnc(-c3noc([C@H]4CC[C@H](CN5CCN(c6ccc7c(c6)CN(C6CCC(=O)NC6=O)C7=O)CC5)CC4)n3)c(Cl)c2)cnc2ccnn12] 1-[5-chloro-6-[5-[4-[[4-[2-(2,6-dioxo-3-piperidyl)-1-oxo-isoindolin-5-yl]piperazin-1- yl]methyl]cyclohexyl]-1,2,4-oxadiazol-3-yl]-3-pyridyl]-3-(7-isopropylpyrazolo[1,5- a]pyrimidin-6-yl)urea |
| 175 | 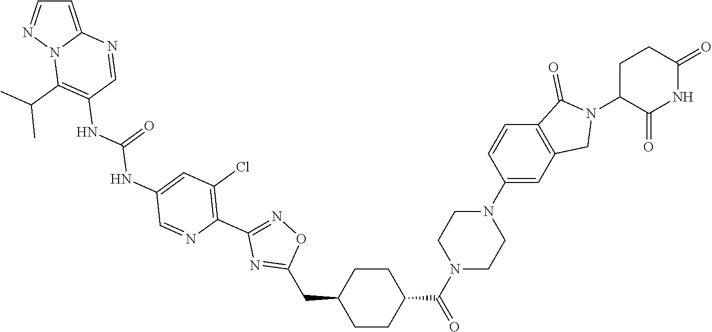 [SMILES: CC(C)c1c(NC(=O)Nc2cnc(-c3noc(C[C@H]4CC[C@H](C(=O)N5CCN(c6ccc7c(c6)CN(C6CCC(=O)NC6=O)C7=O)CC5)CC4)n3)c(Cl)c2)cnc2ccnn12] 1-[5-chloro-6-[5-[[4-[4-[2-(2,6-dioxo-3-piperidyl)-1-oxo-isoindolin-5-yl]piperazine-1- carbonyl]cyclohexyl]methyl]-1,2,4-oxadiazol-3-yl]-3-pyridyl]-3-(7- isopropylpyrazolo[1,5-a]pyrimidin-6-yl)urea |
| 176 | 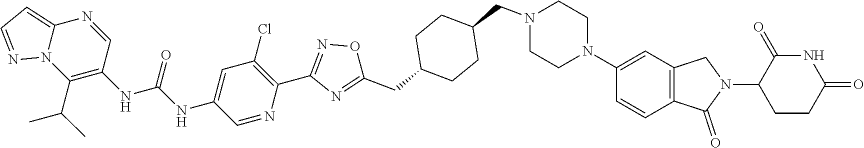 [SMILES: CC(C)c1c(NC(=O)Nc2cnc(-c3noc(C[C@H]4CC[C@H](CN5CCN(c6ccc7c(c6)CN(C6CCC(=O)NC6=O)C7=O)CC5)CC4)n3)c(Cl)c2)cnc2ccnn12] 1-[5-chloro-6-[5-[[4-[[4-[2-(2,6-dioxo-3-piperidyl)-1-oxo-isoindolin-5-yl]piperazin-1- yl]methyl]cyclohexyl]methyl]-1,2,4-oxadiazol-3-yl]-3-pyridyl]-3-(7- isopropylpyrazolo[1,5-a]pyrimidin-6-yl)urea |

In some embodiments, the compound is a pharmaceutically acceptable salt ofany one of the compounds described in Table 1.

In some embodiments, the compound is a lithium salt, sodium salt, potassium salt, calcium salt, or magnesium salt ofany one of the compounds described in Table 1.

In some embodiments, the compound is a sodium salt or potassium salt ofany one of the compounds described in Table 1.

In some embodiments, the compound is a salt of any acid described in the Table 2 and any one of the compounds described in Table 1.

TABLE 2
Pharmaceutical acceptable acid forming
salts with the Compound of Formula (A).

1-hydroxy-2-naphthoic acid
2,2-dichloroacetic acid
2-hydroxyethanesulfonic acid
2-oxoglutaric acid
4-acetamidobenzoic acid
4-aminosalicylic acid
acetic acid
adipic acid
ascorbic acid (L)
aspartic acid (L)
benzenesulfonic acid
benzoic acid
camphoric acid (+)
camphor-10-sulfonic acid (+)
capric acid (decanoic acid)
caproic acid (hexanoic acid)
caprylic acid (octanoic acid)
carbonic acid
cinnamic acid
citric acid
cyclamic acid
dodecylsulfuric acid
ethane-1,2-disulfonic acid
ethanesulfonic acid
formic acid
fumaric acid
galactaric acid
gentisic acid
glucoheptonic acid (D)
gluconic acid (D)
glucuronic acid (D)
glutamic acid
glutaric acid
glycerophosphoric acid
glycolic acid
hippuric acid
hydrobromic acid
hydrochloric acid
isobutyric acid
lactic acid (DL)
lactobionic acid
lauric acid
maleic acid
malic acid (−L)
malonic acid
mandelic acid (DL)
methanesulfonic acid
naphthalene-1,5-disulfonic acid
naphthalene-2-sulfonic acid
nicotinic acid
nitric acid
oleic acid
oxalic acid
palmitic acid
pamoic acid
phosphoric acid
proprionic acid
pyroglutamic acid (−L)
salicylic acid
sebacic acid
stearic acid
succinic acid
sulfuric acid
tartaric acid (+L)
thiocyanic acid
toluenesulfonic acid (p)
undecylenic acid

In some embodiments, the compound is a salt of acetic acid and any one of the compounds described in Table 1.

In some embodiments, the compound is a salt of adipic acid and any one of the compounds described in Table 1.

In some embodiments, the compound is a salt of ascorbic acid (L) and any one of the compounds described in Table 1.

In some embodiments, the compound is a salt of hydrobromic acid and any one of the compounds described in Table 1.

In some embodiments, the compound is a salt of hydrochloric acid and any one of the compounds described in Table 1.

In some embodiments, the compound is a salt of citric acid and any one of the compounds described in Table 1.

In some embodiments, the compound is a salt of glutamic acid and any one of the compounds described in Table 1.

In some embodiments, the compound is a salt of oxalic acid and any one of the compounds described in Table 1.

In some embodiments, the compound is a salt of formic acid and any one of the compounds described in Table 1.

In some embodiments, the compound is a salt of sulfuric acid and any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (A) is isotopically enriched with regard to, or labelled with, one or more isotopes as compared to the corresponding compound of Formula (A). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof).

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the prodrugs of the compounds described in Table 1 and pharmaceutically acceptable salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

In some embodiments, the compound is a ¹⁸F labeled compound.

In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof.

In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the disclosure or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³S, ³⁴S, ³⁵S, and ³⁶S atom(s) is within the scope of the disclosure. Further, substitution with isotope (e.g., ¹⁸F, ¹²³I, ¹²⁴I, ¹²⁵I, ¹²⁹I, ¹³¹I, ¹³⁵I, ³S, ³⁴S, ³S, and/or ³⁶S) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1100 Daltons. More usually, the molecular weight of the compound will be less than 1000, for example less than 900, or less than 800.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) or (II) include all such forms. For the avoidance of doubt, where a compound can exist in one of several tautomeric forms, and only one is specifically described or shown, all others are nevertheless embraced by Formula (I) or (II). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

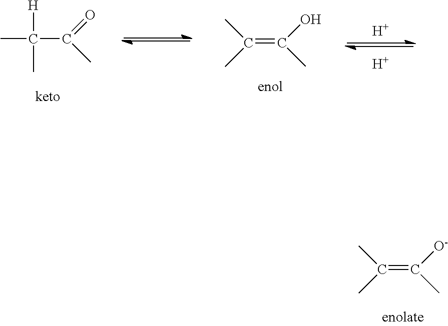

Compounds of any one of the Formulae disclosed herein containing an amine function may also form N-oxides. A reference herein to a compound of Formula (I) or (II) that contains an amine function also includes the N-oxide. Where a compound contains several amine functions, one or more than one nitrogen atom may be oxidized to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄ alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Method of Synthesizing the Compounds

The compounds of the present invention may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (I). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedures A or B which comprise different sequences of assembling intermediates or compounds. Starting materials are either commercially available or made by known procedures in the reported literature or as illustrated below.

### General Procedure

In general, the compound of the Formula (I) can be prepared using sequences of reaction well known to those skilled in the art of organic synthesis. General scheme showed a few ways for the compound of the Formula (I) preparation presented below:

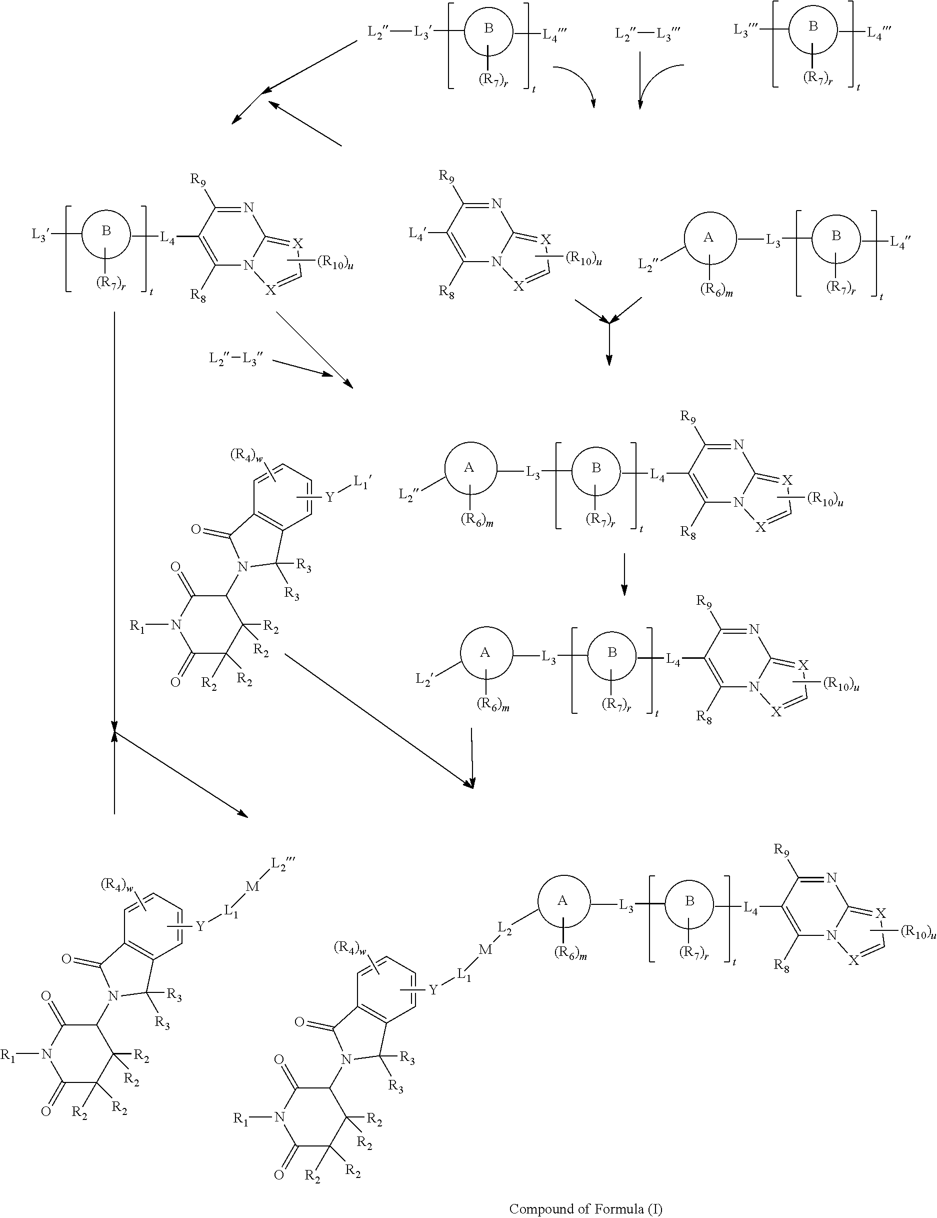

All reagents may be commercially available compounds itself or products of synthesis from commercially available reagents. For each compound in preparation may be used one step or multistep synthetic procedures, including but not limited procedures described herein in preparative part.

As a specific non-limiting example of the sequence of the reaction leading to the compound of Formula (I) can be presented by the preparation of the compound 4:

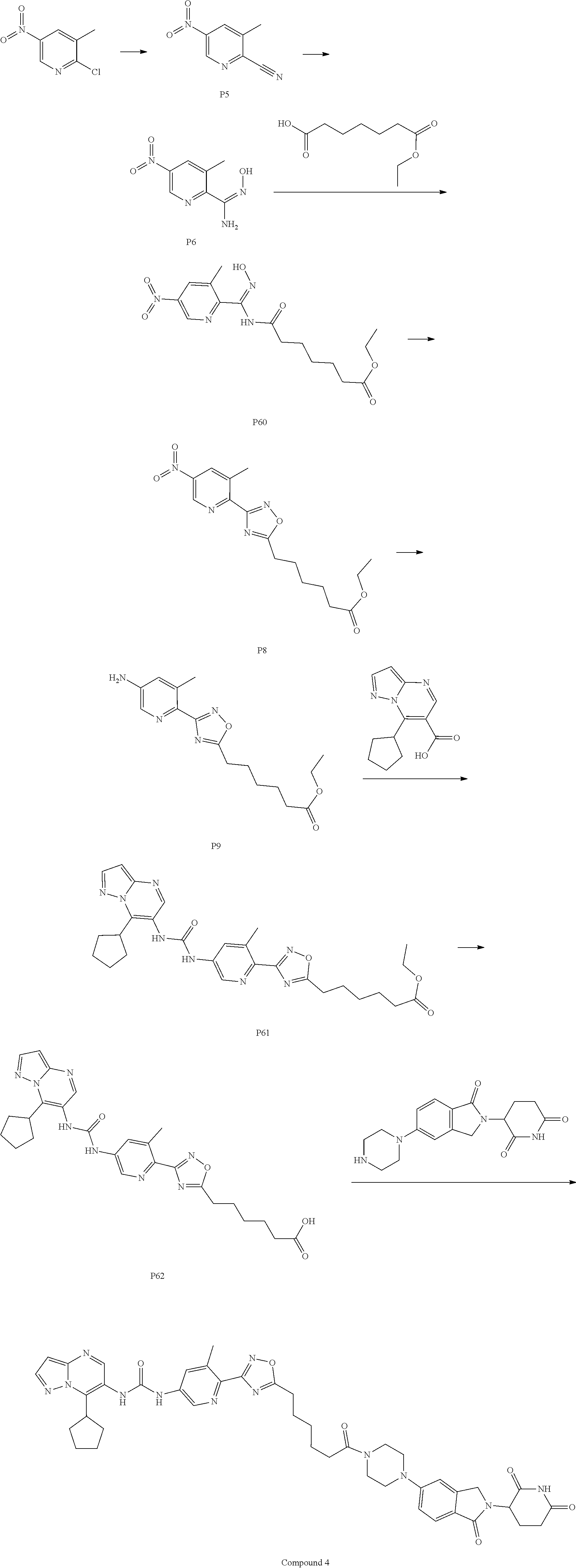

As a specific non-limiting example of the sequence of the reaction leading to the compound of Formula (I) can be presented by the preparation of the compound 15:

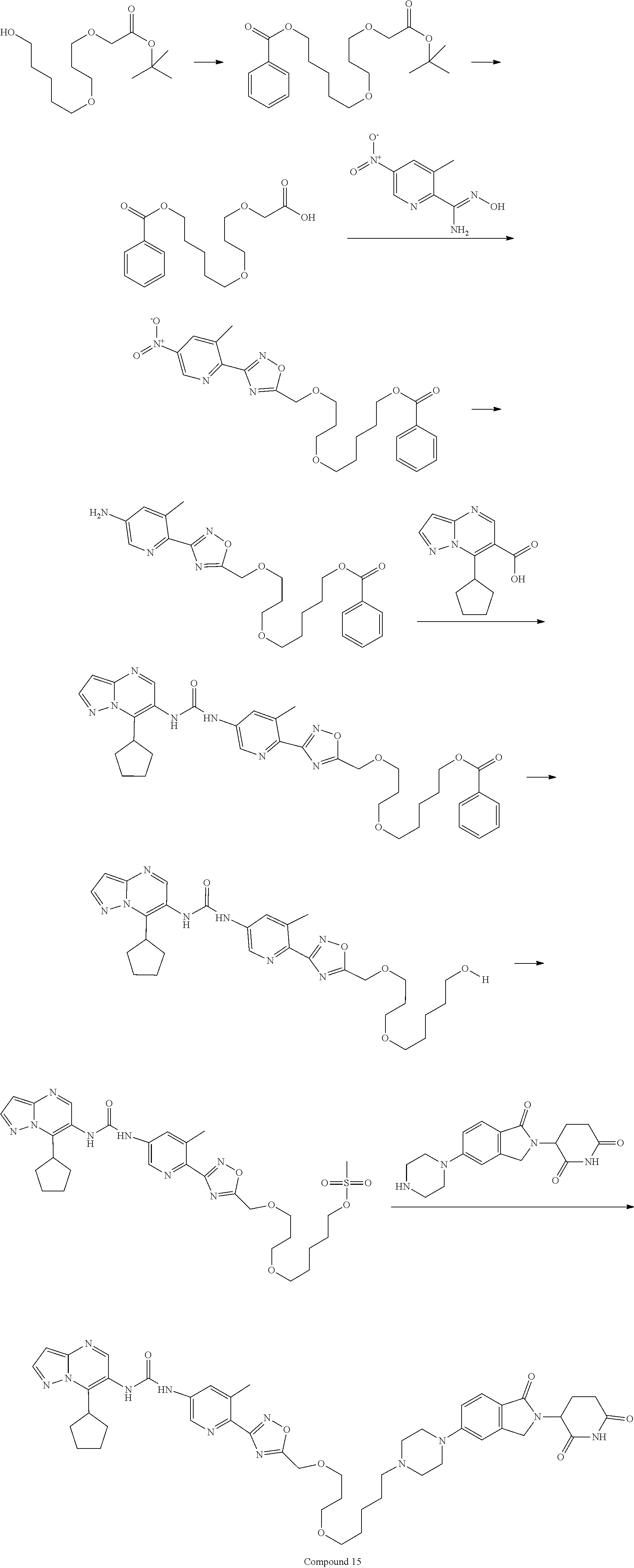

As a specific non-limiting example of the sequence of the reaction leading to the compound of Formula (I) can be presented by the preparation of the compound 16:

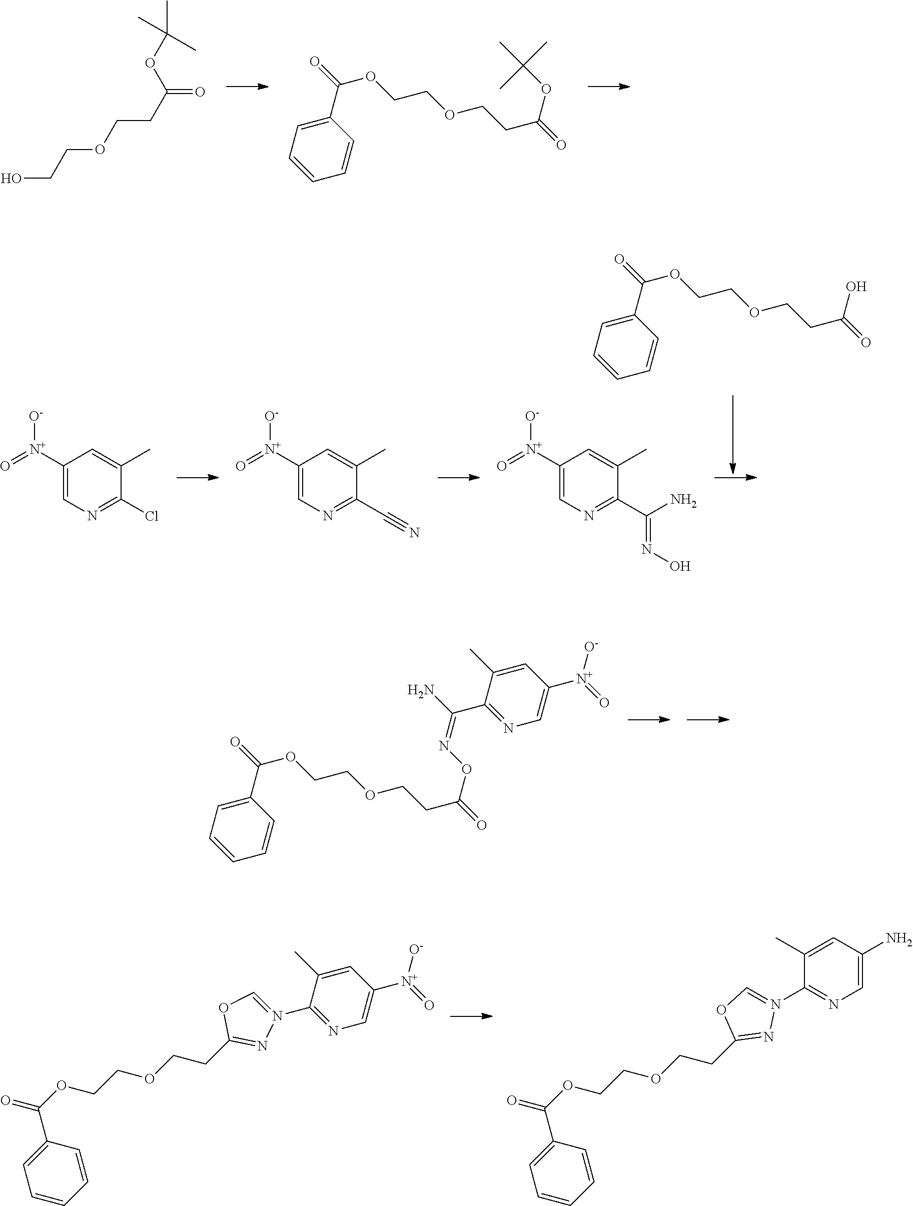

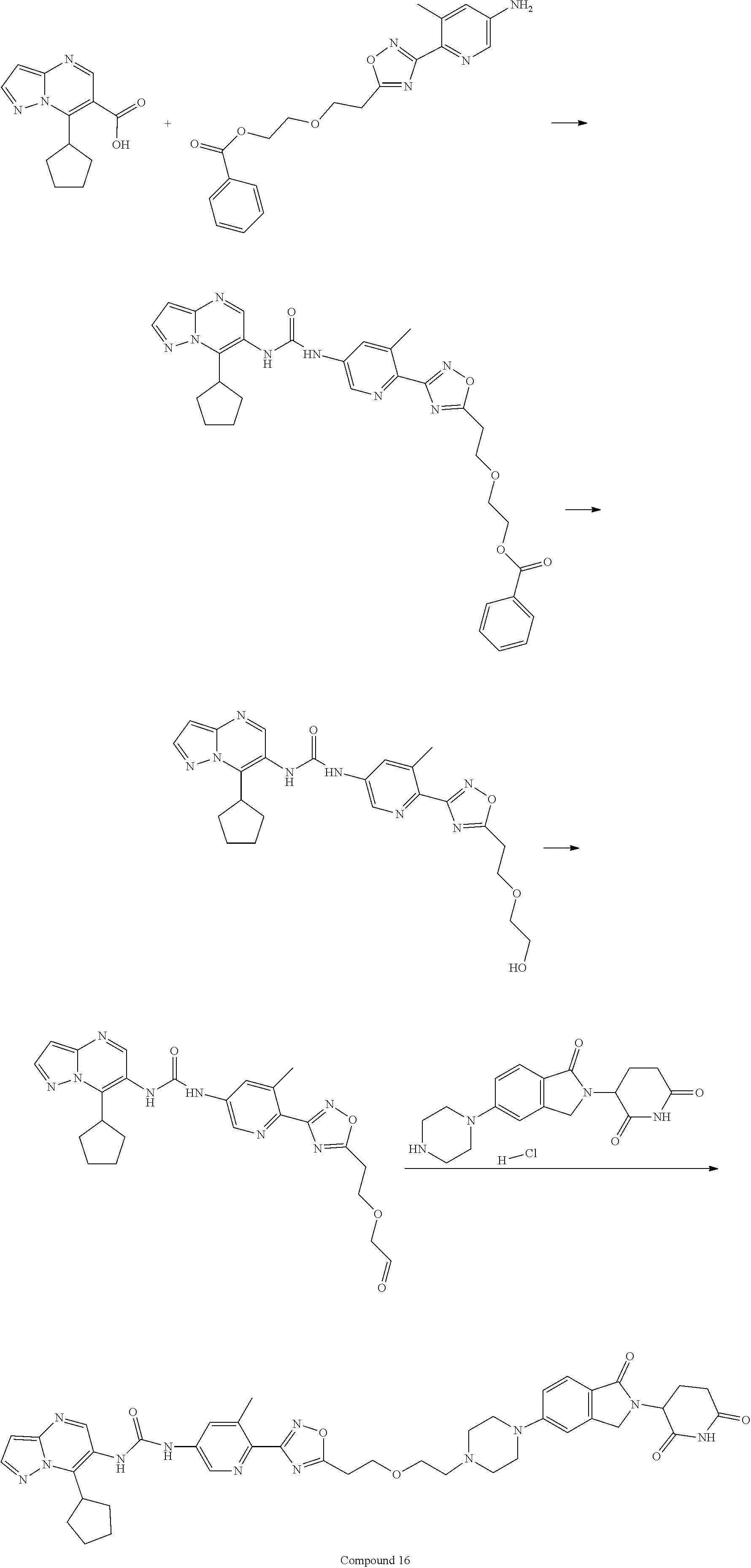

As a specific non-limiting example of the sequence of the reaction leading to the compound of Formula (I) can be presented by the preparation of the compound 6:

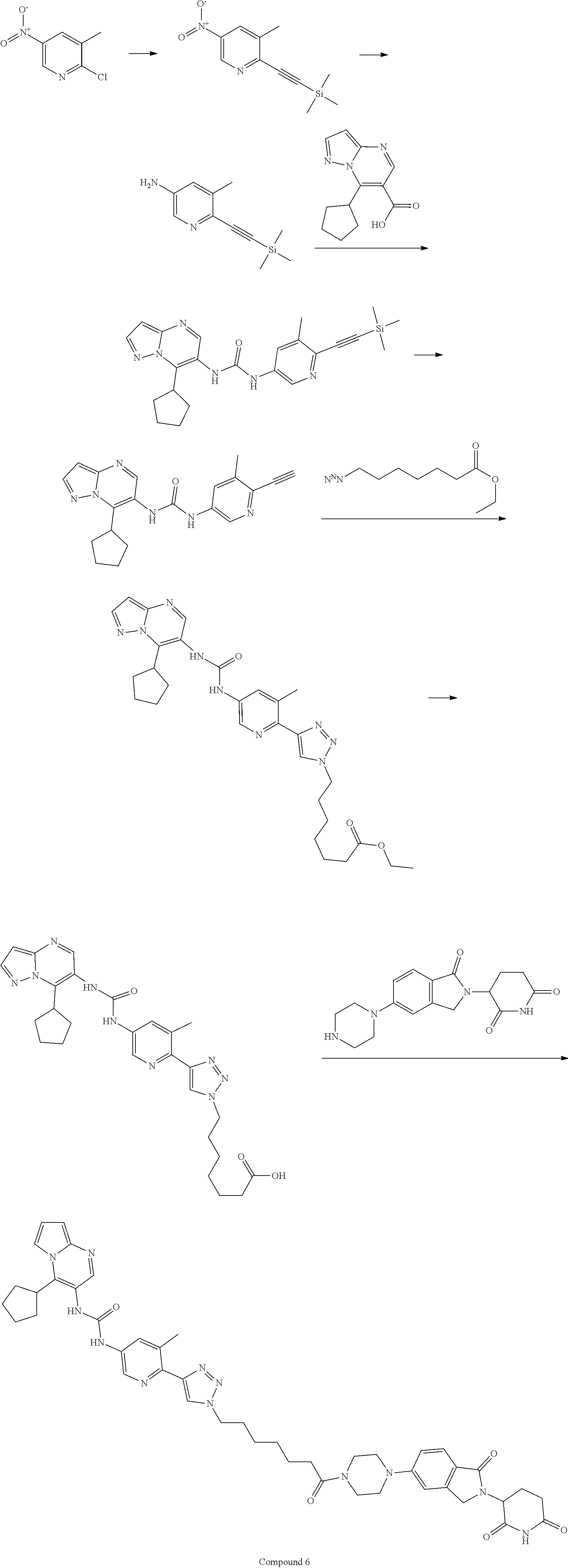

As a specific non-limiting example of the sequence of the reaction leading to the compound of Formula (I) can be presented by the preparation of the compound 19:

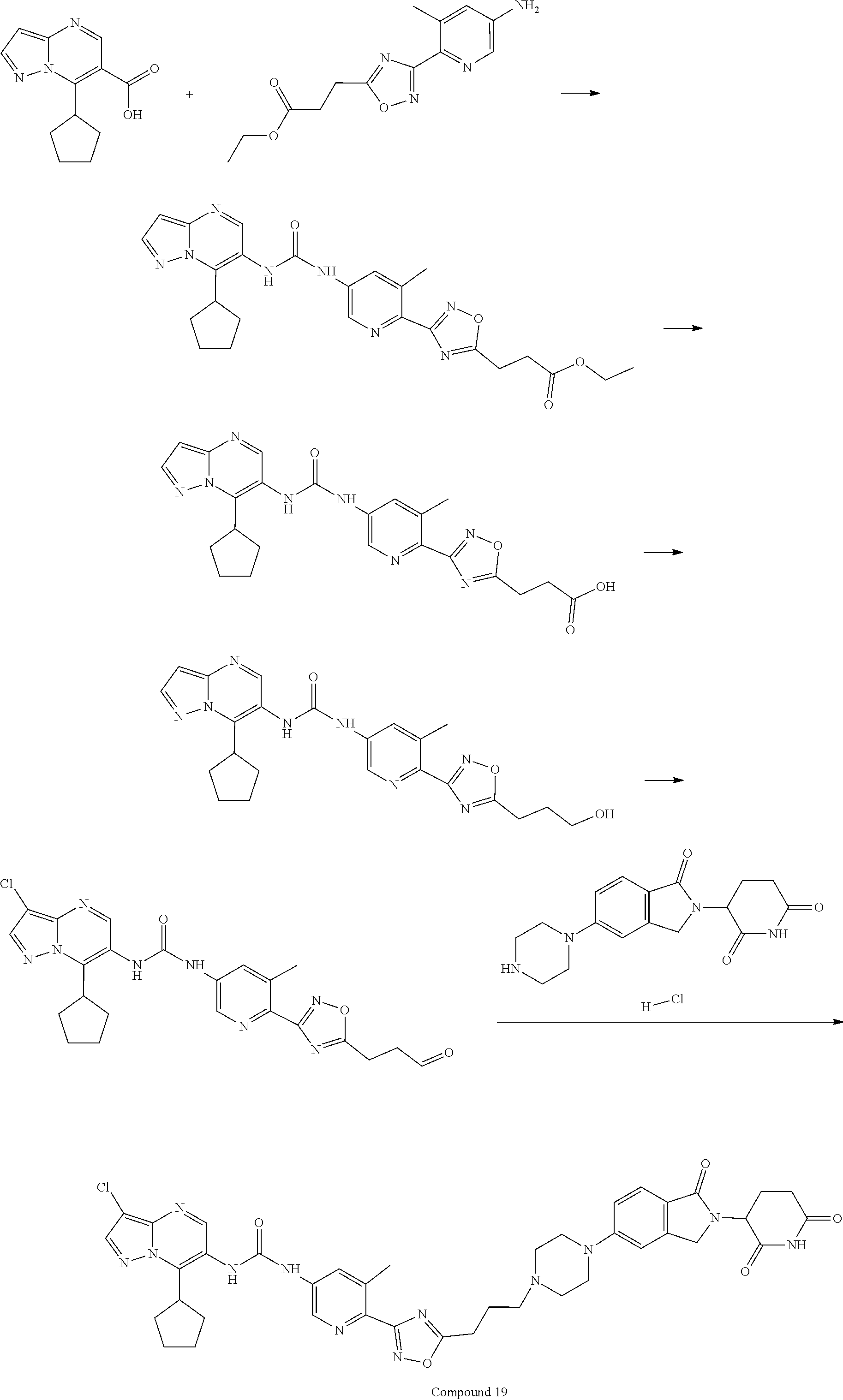

Persons of skill in the art will readily comprehend that the compounds of Formula (I) obtained according to the procedures described above may be a subject for further transformation and modification to obtain other compounds of Formula (I).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a MALT1 related condition referred to herein, slow its progression and/or reduce the symptoms associated with the condition.

A therapeutically effective amount of a compound of the present disclosure for use in therapy is an amount sufficient to treat an MALT1 related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or subject and the route of administration, according to well-known principles of medicine.

### Methods of Use

In some aspects, the present disclosure provides a method of degradation of MALT1 (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with MALT1. In some embodiments, the disease or disorder is a disease or disorder in which MALT1 is implicated.

The compounds of the invention are PROTACs of MALT1.

The compounds of the invention are also useful in treating diseases associated with the MALT1. For example, diseases and conditions treatable according to the methods of the invention include Immunodeficiency 12; Lymphoma, Mucosa-Associated Lymphoid Type (MALTOMA); Combined Immunodeficiency; Combined T and B Cell Immunodeficiency; or Lymphoma.

In some embodiments, the disease or disorder is Lymphoma.

In some embodiments, the disease or disorder is Lymphoma, Mucosa-Associated Lymphoid Type (MALTOMA).

In some embodiments, the disease or disorder is Immunodeficiency 12.

In some embodiments, the disease or disorder is Combined Immunodeficiency.

In some embodiments, the diseases or disorder is Combined T and B Cell Immunodeficiency.

In some aspects, the present disclosure provides a method of treating or preventing a Lymphoma in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Immunodeficiency 12 in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Lymphoma, Mucosa-Associated Lymphoid Type (MALTOMA) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Combined Immunodeficiency in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Combined T and B Cell Immunodeficiency in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Lymphoma in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Immunodeficiency 12 in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Lymphoma, Mucosa-Associated Lymphoid Type (MALTOMA) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Combined Immunodeficiency in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Combined T and B Cell Immunodeficiency in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in degrading of MALT1 (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a Lymphoma in a subject in need thereof.

In some aspects, the present disclosure provides a compound of the present disclosure or a pharmaceutically acceptable salt thereof for use in treating or preventing a Immunodeficiency 12 in a subject in need thereof.

In some aspects, the present disclosure provides a compound of the present disclosure or a pharmaceutically acceptable salt thereof for use in treating or preventing a Lymphoma, Mucosa-Associated Lymphoid Type (MALTOMA) in a subject in need thereof.

In some aspects, the present disclosure provides a compound of the present disclosure or a pharmaceutically acceptable salt thereof for use in treating or preventing a Combined Immunodeficiency in a subject in need thereof.

In some aspects, the present disclosure provides a compound of the present disclosure or a pharmaceutically acceptable salt thereof for use in treating or preventing a Combined T and B Cell Immunodeficiency in a subject in need thereof.

In some aspects, the present disclosure provides a compound of the present disclosure or a pharmaceutically acceptable salt thereof for use in treating a Lymphoma in a subject in need thereof.

In some aspects, the present disclosure provides a compound of the present disclosure or a pharmaceutically acceptable salt thereof for use in treating a Immunodeficiency 12 in a subject in need thereof.

In some aspects, the present disclosure provides a compound of the present disclosure or a pharmaceutically acceptable salt thereof for use in treating a Lymphoma, Mucosa-Associated Lymphoid Type (MALTOMA) in a subject in need thereof.

In some aspects, the present disclosure provides a compound of the present disclosure or a pharmaceutically acceptable salt thereof for use in treating a Combined Immunodeficiency in a subject in need thereof.

In some aspects, the present disclosure provides a compound of the present disclosure or a pharmaceutically acceptable salt thereof for use in treating a Combined T and B Cell Immunodeficiency in a subject in need thereof.

In some aspects, the present disclosure provides use of a compound of the present disclosure or a pharmaceutically acceptable salt thereof in the manufacture of a medicament for degrading of MALT1 (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing a Lymphoma in a subject in need thereof.

In some aspects, the present disclosure provides use of a compound of the present disclosure or a pharmaceutically acceptable salt thereof in the manufacture of a medicament for treating or preventing a Lymphoma, Mucosa-Associated Lymphoid Type (MALTOMA) in a subject in need thereof.

In some aspects, the present disclosure provides use of a compound of the present disclosure or a pharmaceutically acceptable salt thereof in the manufacture of a medicament for treating or preventing a Immunodeficiency 12 in a subject in need thereof.

In some aspects, the present disclosure provides use of a compound of the present disclosure or a pharmaceutically acceptable salt thereof in the manufacture of a medicament for treating or preventing a Combined Immunodeficiency in a subject in need thereof.

In some aspects, the present disclosure provides use of a compound of the present disclosure or a pharmaceutically acceptable salt thereof in the manufacture of a medicament for treating or preventing a Combined T and B Cell Immunodeficiency in a subject in need thereof.

In some aspects, the present disclosure provides use of a compound of the present disclosure or a pharmaceutically acceptable salt thereof in the manufacture of a medicament for treating a Lymphoma in a subject in need thereof.

In some aspects, the present disclosure provides use of a compound of the present disclosure or a pharmaceutically acceptable salt thereof in the manufacture of a medicament for treating a Lymphoma, Mucosa-Associated Lymphoid Type (MALTOMA) in a subject in need thereof.

In some aspects, the present disclosure provides use of a compound of the present disclosure or a pharmaceutically acceptable salt thereof in the manufacture of a medicament for treating a Immunodeficiency 12 in a subject in need thereof.

In some aspects, the present disclosure provides use of a compound of the present disclosure or a pharmaceutically acceptable salt thereof in the manufacture of a medicament for treating a Combined Immunodeficiency in a subject in need thereof.

In some aspects, the present disclosure provides use of a compound of the present disclosure or a pharmaceutically acceptable salt thereof in the manufacture of a medicament for treating a Combined T and B Cell Immunodeficiency in a subject in need thereof.

The present disclosure provides compounds that function as PROTAC of the MALT1 (e.g., in vitro or in vivo). The present disclosure therefore provides a method of degrading of MALT1 in vitro or in vivo, said method comprising contacting a cell with a therapeutically effective amount of a compound, or a pharmaceutically acceptable salt thereof, as defined herein.

In some embodiments, the PROTACs of MALT1 is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which MALT1 is implicated in a subject in need of such treatment, said method comprising administering to said subject a therapeutically effective amount of a compound, or a pharmaceutically acceptable salt thereof, or a pharmaceutical composition as defined herein.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Abbreviations used in the following examples and elsewhere herein are:
AcOH acetic acid
aq. aqueous
Boc₂O di-tert-butyl dicarbonate
br. broad
CDI 1,1′-Carbonyldiimidazole
d duplet
DCM dichloromethane
DIPEA N,N-diisopropylethylamine
DMAA 1,3-dimethylamylamine
DMF N,N-dimethyl formamide
DMSO dimethyl sulfoxide
DPPA diphenylphosphoryl azide
DTT dithiothreitol
ESI electrospray ionization
FBS fetal bovine serum
h hour(s)
HATU (1-[bis(dimethylamino)methylene]-1H-1,2,3-triazolo[4,5-b]pyridinium 3-oxide hexafluorophosphate
HEPES 2-[4-(2-Hydroxyethyl)piperazin-1-yl]ethane-1-sulfonic acid
HPLC high pressure (or performance) liquid chromatography
LCMS liquid chromatography mass spectrometry
m multiplet
M molar
MHz megahertz
min minutes
MsCl methanesulfonyl chloride
NBS N-bromosuccinimide
NMR nuclear magnetic resonance
q quadruplet
rt room temperature
s singlet
STAB sodium triacetoxyborohydride
t temperature, triplet
TBTU 2-(1H-Benzotriazole-1-yl)-1,1,3,3-tetramethylaminium tetrafluoroborate
TEA Triethyl amine
TFA trifluoroacetic acid
THF tetrahydrofuran
TLC thin layer chromatography

### EXAMPLES

### General Synthetical Procedures and Examples of the Compound's Preparation.

### Synthesis of Building Blocks

### Synthesis of 7-(tetrahydrofuran-2-yl)pyrazolo[1,5-a]pyrimidine-6-carboxylic acid (P4)

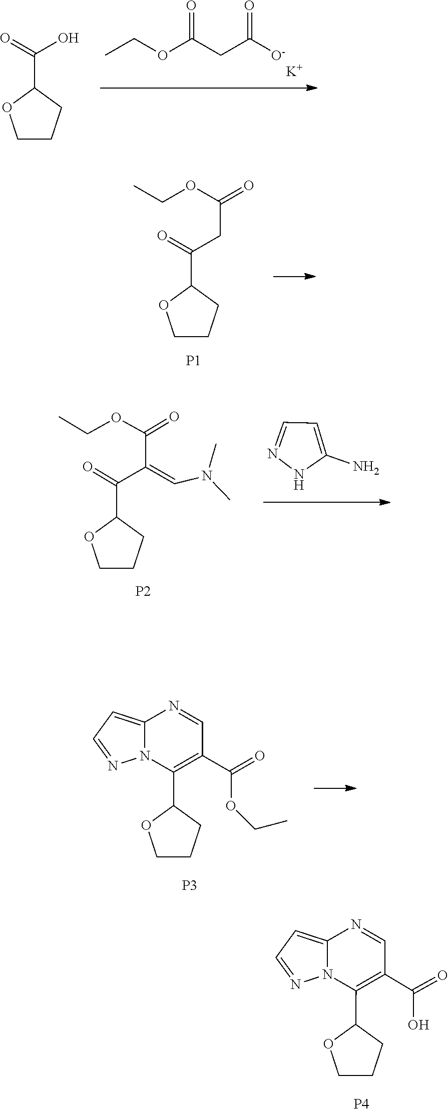

### Preparation 1. Ethyl 3-cyclopentyl-3-oxopropanoate (P1)

1) Carbonyl diimidazole (10.91 g, 67.25 mmol) was added in portions to a mixture of tetrahydrofuran-2-carboxylic acid (7.1 g, 61.14 mmol) and anhydrous EtOAc (100 mL). The mixture was stirred for 3 h at 50° C.

2) MgCl₂ (23.3 g, 122.28 mmol) was added in portions to a mixture of potassium 3-ethoxy-3-oxo-propanoate (20.81 g, 122.28 mmol) and anhydrous EtOAc (300 mL). The mixture was stirred for 3 h at 50° C. The solution from step (1) was added dropwise to the suspension from step (2) and the mixture stirred at reflux overnight. The mixture was cooled in an ice-bath and acidified with 4M HCl (140 mL) to pH=3. The mixture was allowed to warm to ambient temperature and the layers were separated. The aqueous layer was extracted with EtOAc and the combined organic layers were washed with water, brine, and dried (Na₂SO₄). Concentration and purification by chromatography on silica gel gave compound P1 (8.5 g, 75%). ¹H NMR (400 MHz, CDCl₃), δ: 4.40 (dd, J=8.3, 6.5 Hz, 1H), 4.21 (q, J=7.1 Hz, 2H), 3.93 (dd, J=9.6, 3.9 Hz, 2H), 3.59 (s, 2H), 2.21 (ddd, J=15.4, 12.6, 7.1 Hz, 1H), 2.13-2.00 (m, 1H), 1.98-1.85 (m, 2H), 1.26 (t, J=7.1 Hz, 3H).

### Preparation 2. Ethyl (2Z)-3-(dimethylamino)-2-(tetrahydrofuran-2-ylcarbonyl)acrylate (P2)

A solution of compound P1 (10.6 g, 56.9 mmol) and N,N-dimethylformamide dimethyl acetal (75 mL, 569.0 mmol) was stirred at reflux overnight. The mixture was evaporated to dryness and the residue was purified by column chromatography on silica gel. Yield of compound P2 (11.2 g, 82%). ¹H NMR (400 MHz, DMSO-d₆), δ: 7.59 (d, J=8.7 Hz, 1H), 4.80 (dd, J=8.0, 5.4 Hz, 1H), 4.08 (q, J=7.1 Hz, 2H), 3.71 (t, J=6.8 Hz, 2H), 3.31 (s, 6H), 1.99 (dq, J=12.2, 7.7 Hz, 1H), 1.87 (dt, J=12.1, 6.4 Hz, 1H), 1.81-1.66 (m, 2H), 1.21 (t, J=7.1 Hz, 3H).

### Preparation 3. Ethyl 7-(tetrahydrofuran-2-yl)pyrazolo[1,5-a]pyrimidine-6-carboxylate (P3)

To a solution of compound P2 (9.5 g, 39.3 mmol) in 200 mL EtOH, 1H-pyrazol-5-amine (4.0 g, 47.2 mmol) was added. The mixture was stirred at reflux overnight. The mixture was evaporated, and the residue was purified by column chromatography on silica gel. Yield of compound P3 (9.0 g, 88%). ¹H NMR (400 MHz, DMSO-d₆), δ: 8.60 (s, 1H), 8.36 (d, J=2.3 Hz, 1H), 6.86 (t, J=3.9 Hz, 1H), 5.70 (t, J=7.2 Hz, 1H), 4.41-4.32 (m, 1H), 4.31-4.22 (m, 1H), 3.97 (dd, J=14.3, 7.1 Hz, 1H), 3.93-3.83 (m, 1H), 2.67-2.54 (m, 1H), 2.24-1.94 (m, 3H), 1.32 (t, J=7.1 Hz, 3H).

### Preparation 4. 7-(Tetrahydrofuran-2-yl)pyrazolo[1,5-a]pyrimidine-6-carboxylic acid (P4)

A mixture of compound P3 (9.0 g, 34.4 mmol) and NaOH (2.1 g, 51.6 mmol) in 200 mL EtOH and 50 mL H₂O was stirred at 60° C. for 15 h and evaporated. The residue was diluted with water and acidified with 6M HCl to pH=3-4. The formed solid was filtered off and dried. Yield of compound P4 (6.9 g, 86%). ¹H NMR (400 MHz, DMSO-d₆), δ: 13.37 (s, 1H), 8.57 (d, J=10.1 Hz, 1H), 7.96 (t, J=9.5 Hz, 1H), 6.34 (t, J=8.3 Hz, 1H), 5.49 (dd, J=9.0, 4.4 Hz, 1H), 4.06-3.59 (m, 2H), 2.42-2.22 (m, 1H), 1.93-1.78 (m, 2H), 1.77-1.68 (m, 1H).

### Synthesis of Ethyl 6-[3-(5-amino-3-methylpyridin-2-yl)-1,2,4-oxadiazol-5-yl]hexanoate (P9)

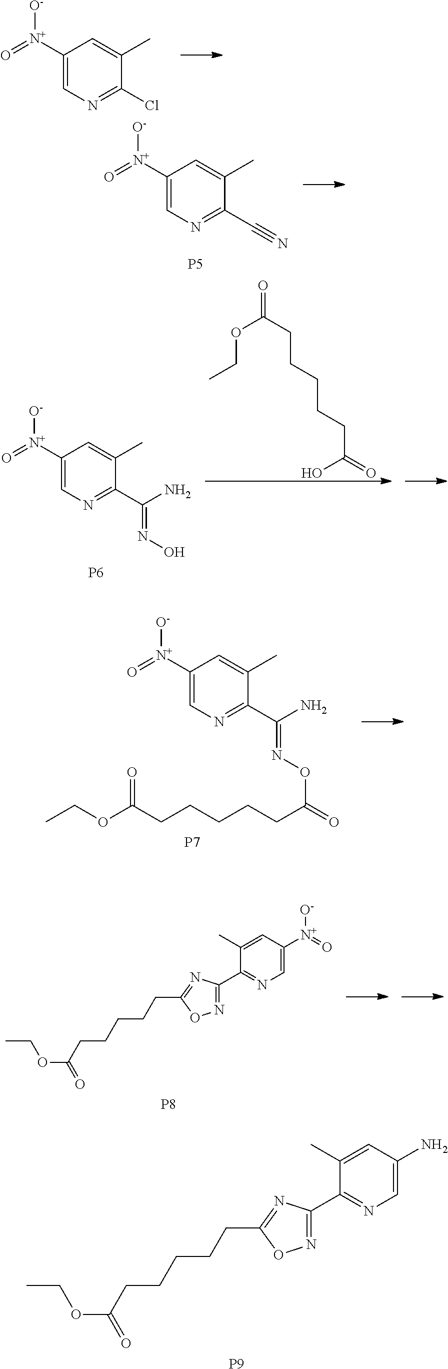

### Preparation 5. 3-Methyl-5-nitropyridine-2-carbonitrile (P5)

A mixture of 2-chloro-3-methyl-5-nitro-pyridine (5.0 g, 29.0 mmol), zinc cyanide (2.7 g, 23.2 mmol), tris (dibenzylideneacetone) dipalladium(0) (1.3 g, 1.45 mmol), and (9,9-dimethyl-9H-xanthene-4,5-diyl)bis(diphenylphosphine) in 1-methyl-2-pyrrolidinone (30 mL) was heated to 130-135° C. for 1 h. The reaction mixture was cooled to ambient temperature, diluted with EtOAc (100 mL) and filtered through Celite. The filtrate was diluted with water and extracted with ethyl acetate. Combined organic extracts were dried with Na₂SO₄ and evaporated. The residue was purified by column chromatography on silica gel. Yield of compound P5 (4.0 g, 85%). ¹H NMR (400 MHz, DMSO-d₆) δ 9.32 (s, 1H), 8.83 (s, 1H), 2.64 (s, 3H).

### Preparation 6. N-Hydroxy-3-methyl-5-nitropyridine-2-carboximidamide (P9)

To a solution of hydroxylamine hydrochloride (8.6 g, 122.5 mmol) in 150 mL H₂O, Na₂CO₃ (6.5 g, 61.3 mmol) was added. The mixture was stirred at ambient temperature for 0.5 h. Compound P5 (4.0 g, 24.5 mmol) was dissolved in EtOH and added to the mixture, and the reaction mixture was stirred at ambient temperature for 14 h. EtOH was evaporated. The formed solid was filtered off and dried. Yield of compound P6 (4.0 g, 83%). ¹H NMR (400 MHz, DMSO-d₆) δ 10.30 (d, J=1.7 Hz, 1H), 9.20 (s, 1H), 8.52 (s, 1H), 5.95 (s, 2H), 2.61 (s, 3H).

### Preparation 7. Ethyl 7-({[(1Z)-amino(3-methyl-5-nitropyridin-2-yl)methylene]amino}oxy)-7-oxoheptanoate (P7)

To a solution of 7-ethoxy-7-oxo-heptanoic acid (0.50 g, 2.7 mmol) in 20 mL of DCM, TEA (1.1 mL, 8.1 mmol) was added. The mixture was stirred at rt for 10 min. After this, TBTU (10.8 g, 3.3 mmol) and Compound P6 (0.52 g, 2.7 mmol) were added, and the mixture was stirred for 15 h at ambient temperature. The mixture was washed with 20% K₂CO₃ (aq.), dried with Na₂SO₄ and evaporated. The residue (P7) was used on the next step without additional purification. LCMS ESI (m/z): 367.4 ([M+1]⁺), 197.3, 179.3.

### Preparation 8. Ethyl 6-[3-(3-methyl-5-nitropyridin-2-yl)-1,2,4-oxadiazol-5-yl]hexanoate (P8)

A solution of compound P7 in 50 mL of dioxane was stirred at reflux for 96 h. The mixture was evaporated and purified by column chromatography on silica gel. Yield of compound P8 on 2 steps (Preparation 7 and Preparation 8) 0.56 g (61%). LCMS ESI (m/z): 349.5 ([M+1]⁺), 303.5.

### Preparation 9. Ethyl 6-[3-(5-amino-3-methylpyridin-2-yl)-1,2,4-oxadiazol-5-yl]hexanoate (P9)

To a solution of compound P8 (0.56 g, 1.6 mmol) in 20 mL acetic acid, Fe powder (0.9 g, 16 mmol) was added. The suspension was stirred at 70° C. for 1 h. The reaction mixture was cooled to ambient temperature, diluted with EtOAc (100 mL) and filtered through Celite. The filtrate was evaporated. The residue was diluted with saturated NaHCO₃ (aq.) and extracted with ethyl acetate. Combined organic extracts were dried with Na₂SO₄ and evaporated. The residue was purified by column chromatography on silica gel. Yield of compound P9 (0.44 g, 86%). LCMS ESI (m/z): 319.4 ([M+1]⁺), 149.4.

### Synthesis of 6-(3-{3-Methyl-5-[({[7-(Tetrahydrofuran-2-yl)pyrazolo[1,5-a]pyrimidin-6-yl]amino}carbonyl)amino]pyridin-2-yl}-1,2,4-oxadiazol-5-yl)hexanoic acid (P11)

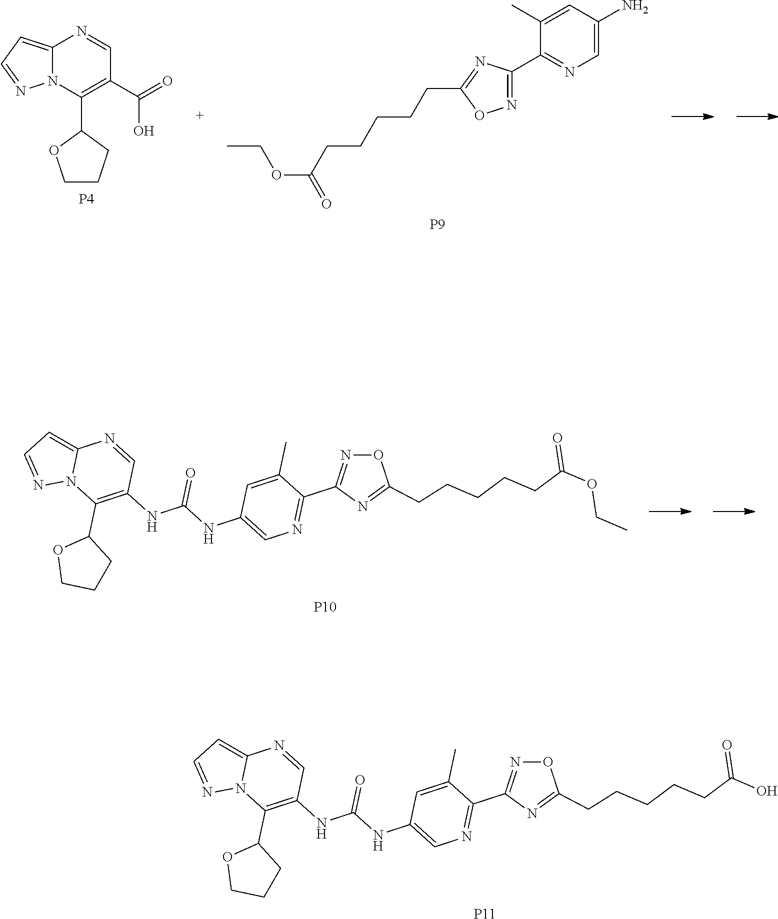

### Preparation 10. Ethyl 6-(3-{3-methyl-5-[({[7-(tetrahydrofuran-2-yl)pyrazolo[1,5-a]pyrimidin-6-yl]amino}carbonyl)amino]pyridin-2-yl}-1,2,4-oxadiazol-5-yl)hexanoate (P10)

To a solution of Compound P4 (0.30 g, 1.3 mmol) in 20 mL of toluene, TEA (0.7 mL, 5.2 mmol) was added. The mixture was stirred at rt for 10 min. Then DPPA (0.34 mL, 1.56 mmol) was added. The mixture was stirred at rt for 1 h, and Compound P9 (0.40 g, 1.3 mmol) was added. The resulting mixture was stirred for 14 h at 90° C. The mixture was evaporated and purified by column chromatography on silica gel. Yield of compound P10 (0.38 g, 54%). LCMS ESI (m/z): 549.7 ([M+1]⁺), 345.1.

### Preparation 11. 6-(3-{3-methyl-5-[({[7-(Tetrahydrofuran-2-yl)pyrazolo[1,5-a]pyrimidin-6-yl]amino}carbonyl)amino]pyridin-2-yl}-1,2,4-oxadiazol-5-yl)hexanoic acid (P11)

A mixture of compound P10 (0.38 g, 0.7 mmol), NaOH (0.05 g, 1.05 mmol) in 10 mL EtOH and 2 mL H₂O was stirred at 60° C. for 15 h and evaporated. The residue was diluted with water and acidified with 6M HCl to pH=3-4. The formed solid was filtered off and dried. Yield of compound P11 (0.36 g, 99%). LCMS ESI (m/z): 521.7 ([M+1]), 317.1.

### Synthesis of 6-{3-[3-Chloro-5-({[(7-cyclopentylpyrazolo[1,5-a]pyrimidin-6-yl)amino]carbonyl}amino)pyridin-2-yl]-1,2,4-oxadiazol-5-yl}hexanoic acid (P18)

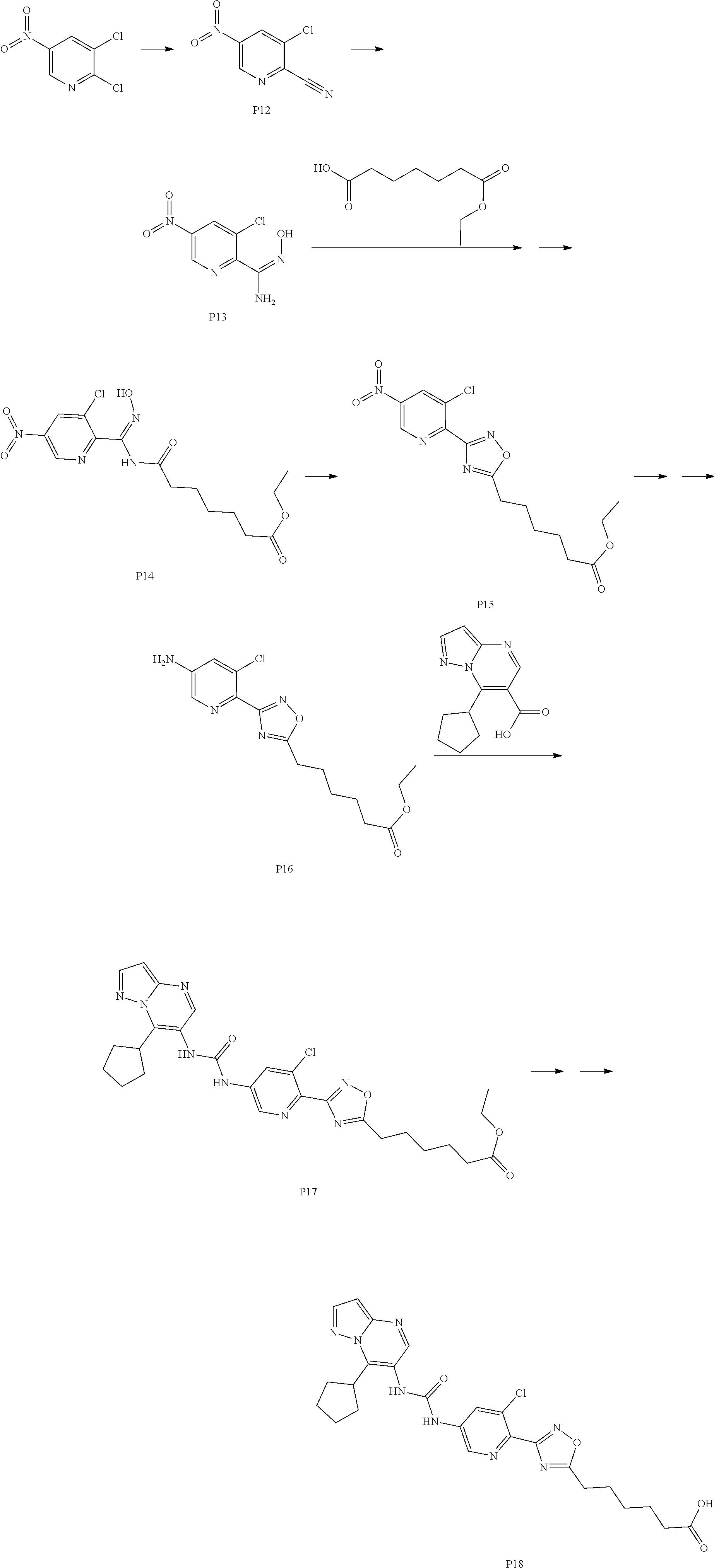

### Preparation 12. 3-Chloro-5-nitropyridine-2-carbonitrile (P12)

Zinc cyanide (1.83 g, 15.55 mmol) was added to a solution of 2,3-dichloro-5-nitropyridine (5.0 g, 25.91 mmol) in 20 mL of N-methyl-2-pyrrolidone. The flask was purged with argon and heated to 135° C. In a separate flask, Tris(dibenzylideneacetone)dipalladium(0) (1.19 g, 1.30 mmol) and 4,5-bis(diphenylphosphino)-9,9-dimethylxanthene (0.75 g, 1.30 mmol) were suspended in 15 mL N-methyl-2-pyrrolidone and the mixture was purged with argon, then heated to 80° C. The suspension was added to the first flask under argon. The reaction mixture was stirred at 135° C. for 1 h. When reaction was completed (TLC monitoring), the reaction mixture was cooled to rt, diluted with ethyl acetate and filtered through Celite. The solution in ethyl acetate was washed with water, dried with Na₂SO₄, and evaporated. The product was purified by column chromatography on silica gel. Eluent—hexane/ethyl acetate=40/1. Product P12 was obtained (3.92 g, 82%) as a solid. ¹H NMR (400 MHz, DMSO-d₆), δ: 9.46 (d, J=2.2 Hz, 2H), 9.17 (d, J=2.2 Hz, 2H).

### Preparation 13. 3-Chloro-N-hydroxy-5-nitropyridine-2-carboximidamide (P13)

A solution of 3-chloro-5-nitropyridine-2-carbonitrile P12 (3.92 g, 21.4 mmol) in 40 mL of ethanol was added dropwise to a solution of hydroxylamine hydrochloride (5.94 g, 85.4 mmol) and Na₂CO₃ (4.53 g, 42.7 mmol) in 110 mL of water. The mixture was stirred at 90° C. for 12 h. The reaction mass was cooled to rt and filtered, giving 3-chloro-N′-hydroxy-5-nitropyridine-2-carboximidamide P13 (4.41 g, 90%). The precipitate was used for further reaction without additional purification. ¹H NMR (400 MHz, DMSO-d₆), δ: 10.22 (s, 1H), 9.31 (d, J=2.2 Hz, 1H), 8.77 (d, J=2.2 Hz, 1H), 6.00 (s, 2H).

### Preparation 14. Ethyl 7-{[(E)-(3-chloro-5-nitropyridin-2-yl)(hydroxyimino)methyl]amino}-7-oxoheptanoate (P14)

To a solution of 7-ethoxy-7-oxoheptanoic acid (0.37 g, 1.94 mmol) in 40 mL of dioxane, N-ethyl-N-isopropylpropan-2-amine (0.30 g, 2.33 mmol) was added. The solution was stirred for 5 min, then P13 (0.42 g, 1.94 mmol) was added, and the mixture was stirred for 10 min. 2,4,6-Tripropyl-1,3,5,2,4,6-trioxatriphosphinane 2,4,6-trioxide (0.93 g, 2.91 mmol, 50 wt % solution in EtOAc) was poured into the reaction flask and the mixture was stirred for 12 h at rt. After completion of the reaction as indicated by TLC, the reaction mixture was concentrated under vacuum, dissolved in ethyl acetate, and washed with water and evaporated. The residue (P14) was used for the next step without additional purification. LCMS ESI (m/z): 387.4 ([M+1]⁺), 217.4.

### Preparation 15. Ethyl 6-[3-(3-chloro-5-nitropyridin-2-yl)-1,2,4-oxadiazol-5-yl]hexanoate (P15)

Ethyl 7-{[(E)-(3-chloro-5-nitropyridin-2-yl)(hydroxyimino)methyl]amino}-7-oxoheptanoate P14 (1.79 g, 4.61 mmol) was dissolved in 50 mL of 1,4-dioxane. The solution was refluxed for 96 h. After completion of the reaction as indicated by TLC, the reaction mixture was concentrated under vacuum. The residue was purified on silica gel eluting with EtOAc/hexane 1:10, giving ethyl 6-[3-(3-chloro-5-nitropyridin-2-yl)-1,2,4-oxadiazol-5-yl]hexanoate P15 (0.62 g). The yield was 37% for two steps (Preparations 14 and 15). LCMS ESI (m/z): 369.1 ([M+1]⁺), 323.5.

### Preparation 16. Ethyl 6-[3-(5-amino-3-chloropyridin-2-yl)-1,2,4-oxadiazol-5-yl]hexanoate (P16)

Ethyl 6-[3-(3-chloro-5-nitropyridin-2-yl)-1,2,4-oxadiazol-5-yl]hexanoate P15 (0.62 g, 1.69 mmol) was dissolved in 50 mL of a solvent consisting of ethanol and acetic acid in equal amounts. The mixture was heated to 40° C. and five equivalents of Fe powder (0.47 g, 8.45 mmol) were added. The stirring was continued at this temperature until the starting substance was completely converted as indicated by TLC. The mixture was cooled down to ambient temperature and passed through Celite. The filtrate was evaporated, and the residue was dissolved in ethyl acetate and washed with water and concentrated. Then the mixture was dissolved in methylene chloride, washed with an aqueous solution of sodium bicarbonate, dried over sodium sulfate and concentrated, giving ethyl 6-[3-(5-amino-3-chloropyridin-2-yl)-1,2,4-oxadiazol-5-yl]hexanoate P16 (0.46 g, 80%). ¹H NMR (400 MHz, DMSO-d₆), δ: 7.99 (d, J=2.3 Hz, 1H), 7.09 (d, J=2.3 Hz, 1H), 6.19 (s, 2H), 4.03 (q, J=7.1 Hz, 2H), 2.97 (t, J=7.4 Hz, 2H), 2.28 (t, J=7.3 Hz, 2H), 1.83-1.69 (m, 2H), 1.67-1.49 (m, 2H), 1.37 (m, 2H), 1.26 (m, 2H), 1.10 (t, J=7.1 Hz, 3H).

### Preparation 17. Ethyl 6-{3-[3-chloro-5-({[(7-cyclopentylpyrazolo[1,5-a]pyrimidin-6-yl)amino]carbonyl}amino)pyridin-2-yl]-1,2,4-oxadiazol-5-yl}hexanoate (P17)

Triethylamine (0.548 g, 5.4 mmol) was added to a suspension of 7-cyclopentylpyrazolo[1,5-a]pyrimidine-6-carboxylic acid (0.31 g, 1.35 mmol) in 40 mL of toluene. The mixture was stirred for 5 min. Then DPPA (0.48 g, 1.62 mmol) was added and stirred for 60 min at rt. After this ethyl 6-[3-(5-amino-3-chloropyridin-2-yl)-1,2,4-oxadiazol-5-yl]hexanoate P16 (0.458 g, 1.35 mmol) was added, and the mixture was refluxed for 12 h. Reaction was monitored by LCMS. The mixture was quenched with water, separated toluene was dried with Na₂SO₄ and evaporated. The residue was purified on silica gel eluting with hexane/ethyl acetate 1:1, giving ethyl 6-{3-[3-chloro-5-({[(7-cyclopentylpyrazolo[1,5-a]pyrimidin-6-yl)amino]carbonyl}amino)pyridin-2-yl]-1,2,4-oxadiazol-5-yl}hexanoate P17 (0.55 g, 72%). LCMS ESI (m/z): 567.5 ([M+1]⁺).

### Preparation 18. 6-{3-[3-Chloro-5-({[(7-cyclopentylpyrazolo[1,5-a]pyrimidin-6-yl)amino]carbonyl}amino)pyridin-2-yl]-1,2,4-oxadiazol-5-yl}hexanoic acid (P18)

Aqueous solution of NaOH (0.08 g, 1.94 mmol) was added to a solution of ethyl 6-{3-[3-chloro-5-({[(7-cyclopentylpyrazolo[1,5-a]pyrimidin-6-yl)amino]carbonyl}amino)pyridin-2-yl]-1,2,4-oxadiazol-5-yl}hexanoate P17 (0.55 g, 0.97 mmol) in 10 ml of ethanol. The mixture was stirred for 12 h at 50° C. After the full hydrolysis of the ester, the mixture was cooled down to ambient temperature. Ethanol was evaporated and the reaction mixture was acidified with hydrochloric acid to pH=7, the product was then extracted with ethyl acetate. Solution of the product in ethyl acetate was dried with Na₂SO₄ and evaporated. 6-{3-[3-chloro-5-({[(7-cyclopentylpyrazolo[1,5-a]pyrimidin-6-yl)amino]carbonyl}amino)pyridin-2-yl]-1,2,4-oxadiazol-5-yl}hexanoic acid P18 (0.378 g, 72%) was obtained as a solid. LCMS ESI (m/z): 539.5 ([M+1]⁺), 431.5.

### Synthesis of 6-{5-[5-({[(7-Cyclopentylpyrazolo[1,5-a]pyrimidin-6-yl)amino]carbonyl}amino)-3-methylpyridin-2-yl]-1,3,4-oxadiazol-2-yl}hexanoic acid (P26)

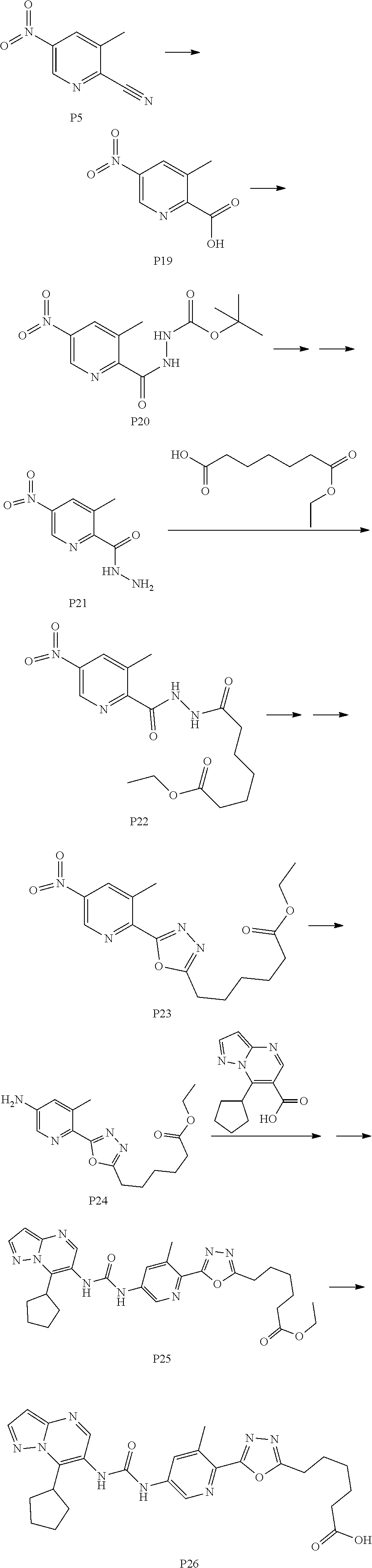

### Preparation 19. 3-Methyl-5-nitropyridine-2-carboxylic acid (P19)

In a 25 mL microwave vial, 3-methyl-5-nitropyridine-2-carbonitrile P5 (1.75 g, 10.7 mmol) was dissolved in a mixture of EtOH (5 mL) and conc. H₂SO₄ (15 mL). The vial was sealed and heated to 120° C. in a microwave oven for 60 min. After completion, the reaction mixture was cooled to rt and concentrated to dryness. The obtained residue was dissolved in THF and 1N NaOH was added slowly up to pH=9. The resulting mixture was stirred at 50° C. for 1 h. The mixture was then diluted with water and acidified to pH 4-5 using 1 N HCl and then extracted with EtOAc. The EtOAc layer was washed with brine (10 mL), dried (Na₂SO₄), and concentrated to provide the crude product which was used in the next step without purification. Yield of 3-methyl-5-nitropyridine-2-carboxylic acid P19 was 1.05 g (53%). ¹H NMR (400 MHz, DMSO-d₆), δ: 11.90 (br. s, 1H), 9.46-8.99 (m, 1H), 8.60 (d, J=17.4 Hz, 1H), 2.54 (s, 3H).

### Preparation 20. tert-Butyl 2-[(3-methyl-5-nitropyridin-2-yl)carbonyl]hydrazinecarboxylate (P20)

To a solution of the 3-methyl-5-nitropyridine-2-carboxylic acid P19 (1.56 g, 8.57 mmol) in ml of N,N-dimethylformamide was added N-[(dimethylamino)(3-oxido-1H-[1,2,3]triazolo[4,5-b]pyridin-1-yl)methylene]-N-methylmethanaminium hexafluorophosphate (4.89 g, 12.85 mmol), and the solution was allowed to mix for 5 min. Then tert-butyl carbazate (1.36 g, 10.28 mmol) and triethylamine (1.73 g, 17.14 mmol) were added at rt. The reaction mixture was stirred at that temperature for 2 hours. Then the mixture was diluted with water and then extracted with EtOAc. The EtOAc layer was washed with brine (10 mL), dried (Na₂SO₄), and concentrated, giving tert-butyl 2-[(3-methyl-5-nitropyridin-2-yl)carbonyl]hydrazinecarboxylate (1.78 g, 70%). ¹H NMR (400 MHz, CDCl₃), δ: 9.46 (s, 1H), 9.20 (s, 1H), 8.42 (s, 1H), 6.69 (s, 1H), 2.90-2.80 (m, 3H), 1.52 (s, 9H).

### Preparation 21. 3-Methyl-5-nitropyridine-2-carbohydrazide (P21)

tert-Butyl 2-[(3-methyl-5-nitropyridin-2-yl)carbonyl]hydrazinecarboxylate P20 (1.76 g, 5.94 mmol) was dissolved in 20 mL of 1,4-dioxane. Hydrogen chloride, 3N solution in 1,4-dioxane (20 ml) was added. The solution was stirred at rt for 12 h. After completion of the reaction as indicated by LCMS, the reaction mixture was concentrated under vacuum, and residue was dissolved in methanol. Potassium carbonate was added, the mixture was stirred for 5 min, filtered out and concentrated, giving 3-methyl-5-nitropyridine-2-carbohydrazide P21 (460 mg, 40%). LCMS ESI (m/z): 197.2 ([M+1]⁺).

### Preparation 22. Ethyl 7-{2-[(3-methyl-5-nitropyridin-2-yl)carbonyl]hydrazino}-7-oxoheptanoate (P22)

7-Ethoxy-7-oxoheptanoic acid (0.44 g, 2.35 mmol) and N,N-carbonyldiimidazole (0.456 g, 2.82 mmol) were stirred in 20 mL of 1,4-dioxane at 50° C. for 3 hours. Then 3-methyl-5-nitropyridine-2-carbohydrazide P21 (0.46 g, 2.35 mmol) was added and stirred at this temperature for another 12 h. After the reaction was completed, the mixture was diluted with water and extracted with ethyl acetate. Organic extract was dried with Na₂SO₄ and evaporated. The residue was purified by column chromatography on silica gel (eluent—EtOAc/hexane, 5:1), giving ethyl 7-{2-[(3-methyl-5-nitropyridin-2-yl)carbonyl]hydrazino}-7-oxoheptanoate P22 (0.23 g, 27%). LCMS ESI (m/z): 367.5 ([M+1]⁺).

### Preparation 23. Ethyl 6-[5-(3-methyl-5-nitropyridin-2-yl)-1,3,4-oxadiazol-2-yl]hexanoate (P23)

To an iodine (40.9 mg, 0.16 mmol) solution in DCM at 0-5° C., triphenylphosphine was added portionwise (42.2 mg, 0.16 mmol). Then Et₃N was added (32.6 mg, 0.32 mmol) following by addition of ethyl 7-{2-[(3-methyl-5-nitropyridin-2-yl)carbonyl]hydrazino}-7-oxoheptanoate P22 (118 mg, 0.32 mmol). The reaction mixture was stirred overnight at r temperature. The mixture was washed with a 10% sodium thiosulfate solution, dried with Na₂SO₄, and evaporated. The residue was purified by column chromatography on silica gel, giving ethyl 6-[5-(3-methyl-5-nitropyridin-2-yl)-1,3,4-oxadiazol-2-yl]hexanoate P23 (51 mg, 45%). LCMS ESI (m/z): 349.5 ([M+1]⁺).

### Preparation 24. Ethyl 6-[5-(5-amino-3-methylpyridin-2-yl)-1,3,4-oxadiazol-2-yl]hexanoate (P24)

Ethyl 6-[5-(3-methyl-5-nitropyridin-2-yl)-1,3,4-oxadiazol-2-yl]hexanoate P23 (0.2 g, 0.57 mmol) was dissolved in 20 mL of a solvent consisting of ethanol and acetic acid in equal amounts. The mixture was heated to 40° C. and Fe powder (0.16 g, 2.85 mmol) were added. The stirring was continued at this temperature until the starting material was completely converted as indicated by TLC. The mixture was cooled down to ambient temperature and passed through Celite. The filtrate was evaporated, and the residue was dissolved in ethyl acetate and washed with water and concentrated. Then the mixture was dissolved in methylene chloride, washed with an aqueous solution of sodium bicarbonate, dried over sodium sulfate and concentrated, giving ethyl 6-[5-(5-amino-3-methylpyridin-2-yl)-1,3,4-oxadiazol-2-yl]hexanoate P24 (0.15 g, 82%). LCMS ESI (m/z): 319.5 ([M+1]⁺).

### Preparation 25. Ethyl 6-{5-[5-({[(7-cyclopentylpyrazolo[1,5-a]pyrimidin-6-yl)amino]carbonyl}amino)-3-methylpyridin-2-yl]-1,3,4-oxadiazol-2-yl}hexanoate (P25)

Triethylamine (0.189 g, 1.87 mmol) was added to a suspension of 7-cyclopentylpyrazolo[1,5-a]pyrimidine-6-carboxylic acid (0.108 g, 0.46 mmol) in 20 ml of toluene. The mixture was stirred for 5 min. Then DPPA (0.15 g, 0.56 mmol) was added and stirred for 60 min at rt. After this, ethyl 6-[5-(5-amino-3-methylpyridin-2-yl)-1,3,4-oxadiazol-2-yl]hexanoate P24 (0.189 g, 0.56 mmol) was added, and the mixture was refluxed for 12 h. Reaction was monitored by LCMS. The mixture was quenched with water, separated toluene layer was dried with Na₂SO₄ and evaporated. The residue was purified on silica gel eluting with hexane/ethyl acetate, 1:1, giving ethyl 6-{5-[5-({[(7-cyclopentylpyrazolo[1,5-a]pyrimidin-6-yl)amino]carbonyl}amino)-3-methylpyridin-2-yl]-1,3,4-oxadiazol-2-yl}hexanoate P25 (0.165 g, 62%). LCMS ESI (m/z): 547.6 ([M+1]⁺).

### Preparation 26. 6-{5-[5-({[(7-Cyclopentylpyrazolo[1,5-a]pyrimidin-6-yl)amino]carbonyl}amino)-3-methylpyridin-2-yl]-1,3,4-oxadiazol-2-yl}hexanoic acid (P26)

Aqueous solution of NaOH (8.2 mg, 0.2 mmol) was added to a solution ethyl 6-{5-[5-({[(7-cyclopentylpyrazolo[1,5-a]pyrimidin-6-yl)amino]carbonyl}amino)-3-methylpyridin-2-yl]-1,3,4-oxadiazol-2-yl}hexanoate P25 (56 mg, 0.1 mmol) in 10 ml of ethanol. The mixture was stirred for 12 h at 50° C. After hydrolysis of the ester was completed, the mixture was cooled down to ambient temperature. Ethanol was evaporated and the reaction mixture was acidified with hydrochloric acid to pH=7, the product was then extracted with ethyl acetate. Solution of the product in ethyl acetate was dried with Na₂SO₄ and evaporated. 6-{5-[5-({[(7-cyclopentylpyrazolo[1,5-a]pyrimidin-6-yl)amino]carbonyl}amino)-3-methylpyridin-2-yl]-1,3,4-oxadiazol-2-yl}hexanoic acid P26 (47.6 mg, 90%) was obtained as a solid. LCMS ESI (m/z): 519.6 ([M+1]⁺).

### Synthesis of 7-{4-[5-({[(7-Cyclopentylpyrazolo[1,5-a]pyrimidin-6-yl)amino]carbonyl}amino)-3-methylpyridin-2-yl]-1H-1,2,3-triazol-1-yl}heptanoic acid (P32)

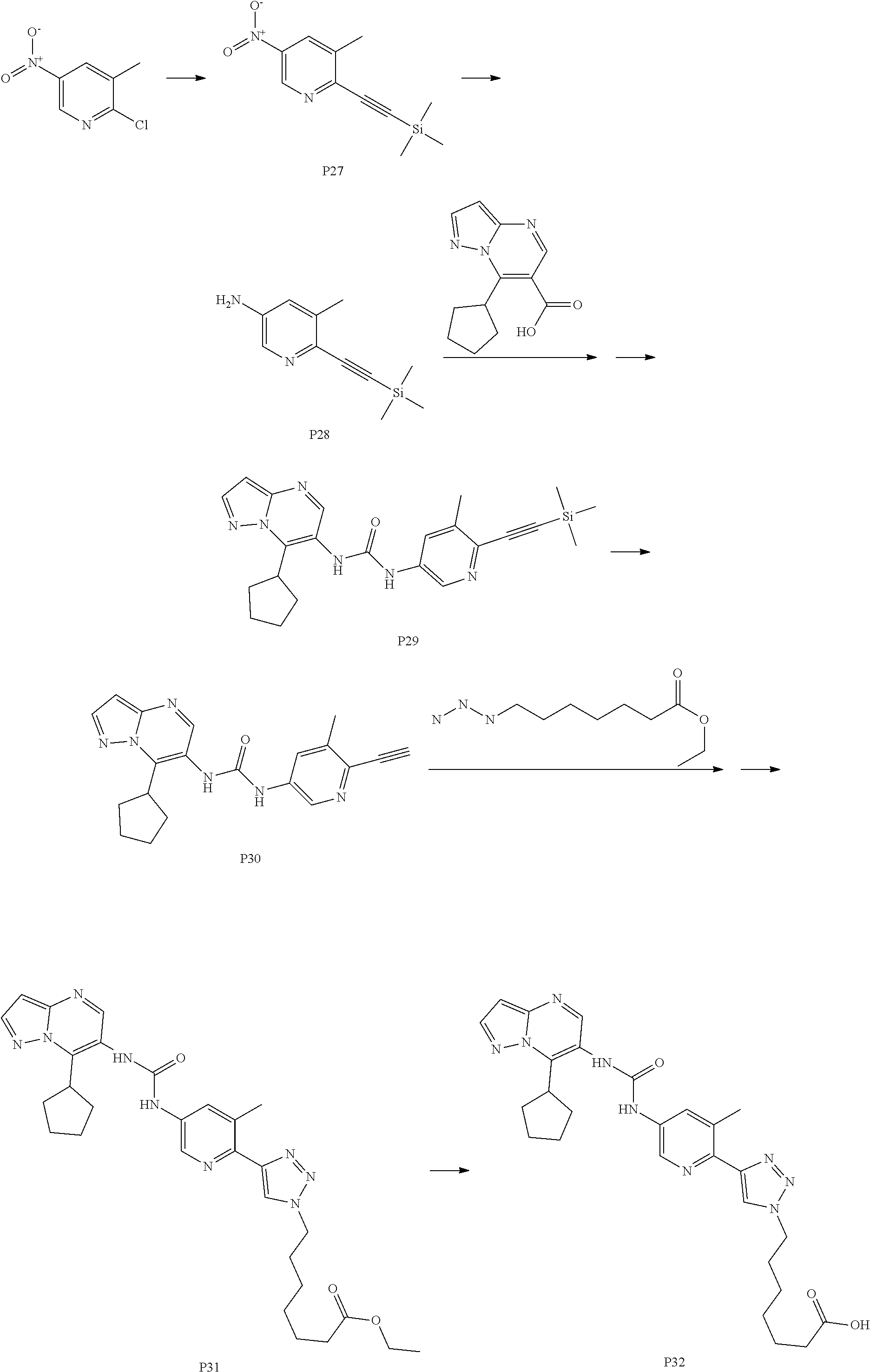

### Preparation 27. 3-Methyl-5-nitro-2-[(trimethylsilyl)ethynyl]pyridine (P27)

Ethynyltrimethylsilane (2.56 g, 26.08 mmol) was added to suspension of 2-chloro-3-methyl-5-nitropyridine (3.0 g, 13.78 mmol), tetrakis(triphenylphosphine)palladium(0) (2.01 g, 1.74 mmol), and CuI powder (0.66 g, 3.48 mmol) in triethylamine (100 mL). The flask was purged with argon and heated at 80° C. for 12 h. When reaction was completed (TLC monitoring), the reaction mixture was cooled to rt, diluted with ethyl acetate and filtered through Celite. The filtrate was washed with saturated aqueous solutions of NaHCO₃ and NaCl, dried with Na₂SO₄ and concentrated. The product was purified by column chromatography on silica gel. Eluent—hexane/ethyl acetate, 80:1. 3-Methyl-5-nitro-2-[(trimethylsilyl)ethynyl]pyridine P27 (1.14 g, 28%) was obtained as a solid. ¹H NMR (400 MHz, CDCl₃) δ 9.23 (d, J=2.2 Hz, 1H), 8.33 (d, J=2.0 Hz, 1H), 2.57 (s, 3H), 0.33 (s, 9H).

### Preparation 28. 5-Methyl-6-[(trimethylsilyl)ethynyl]pyridin-3-amine (P28)

3-Methyl-5-nitro-2-[(trimethylsilyl)ethynyl]pyridine P27 (1.13 g, 4.82 mmol) was dissolved in 50 mL of a solvent consisting of ethanol and acetic acid in equal amounts. The mixture was heated to 40° C. and five equivalents of Fe powder (1.35 g, 24.1 mmol) were added. The stirring was continued at this temperature until the starting substance was completely converted as indicated by TLC. The mixture was cooled down to ambient temperature and passed through Celite. The filtrate was evaporated, and the residue was dissolved in ethyl acetate and washed with water and concentrated. Then the mixture was dissolved in methylene chloride, washed with an aqueous solution of sodium bicarbonate, dried over sodium sulfate and concentrated, giving 5-methyl-6-[(trimethylsilyl)ethynyl]pyridin-3-amine P28 (0.87 g, 88%). ¹H NMR (400 MHz, CDCl₃), δ: 7.92 (d, J=2.3 Hz, 1H), 7.00 (br.s, 2H), 6.80 (d, J=2.0 Hz, 1H), 2.35 (s, 3H), 0.26 (s, 9H).

### Preparation 29. N-(7-Cyclopentylpyrazolo[1,5-a]pyrimidin-6-yl)-N′-{5-methyl-6-[(trimethylsilyl)ethynyl]pyridin-3-yl}urea (P29)

Triethylamine (1.44 g, 14.2 mmol) was added to a suspension of 7-cyclopentylpyrazolo[1,5-a]pyrimidine-6-carboxylic acid (0.82 g, 3.55 mmol) in 40 mL of toluene. The mixture was stirred for 5 min. Then DPPA (1.17 g, 4.26 mmol) was added and stirred for 60 min at rt. After this, 5-methyl-6-[(trimethylsilyl)ethynyl]pyridin-3-amine P28 (0.87 g, 4.26 mmol) was added, and the mixture was refluxed for 12 h. Reaction was monitored by LCMS. The mixture was quenched with water, separated toluene was dried with Na₂SO₄ and evaporated. The residue was purified on silica gel eluting with hexane/ethyl acetate, 1:1, giving N-(7-cyclopentylpyrazolo[1,5-a]pyrimidin-6-yl)-N′-{5-methyl-6-[(trimethylsilyl)ethynyl]pyridin-3-yl}urea P29 (0.47 g, 30%). ¹H NMR (400 MHz, DMSO-d₆), δ: 9.22 (s, 1H), 8.45 (br. s, 3H), 8.22 (d, J=2.4 Hz, 1H), 7.87 (s, 1H), 6.74 (d, J=2.2 Hz, 1H), 3.97-3.74 (m, 1H), 2.34 (s, 3H), 2.36-2.32 (m, 2H), 1.99 (s, 2H), 1.85 (s, 2H), 1.70 (s, 2H), 0.25 (s, 9H).

### Preparation 30. N-(7-Cyclopentylpyrazolo[1,5-a]pyrimidin-6-yl)-N-(6-ethynyl-5-methylpyridin-3-yl)urea (P30)

N-(7-cyclopentylpyrazolo[1,5-a]pyrimidin-6-yl)-N′-{5-methyl-6-[(trimethylsilyl)ethynyl]pyridin-3-yl}urea P29 (0.24 g, 0.56 mmol) was dissolved in 20 mL of a solvent consisting of methanol and dichloromethane in equal amounts. Potassium fluoride (0.097 g, 1.67 mmol) was added in the flask, and it was stirred for 2 h at rt. After completion of the reaction as indicated by TLC, the reaction mass was diluted with water and extracted with ethyl acetate. N-(7-Cyclopentylpyrazolo[1,5-a]pyrimidin-6-yl)-N-(6-ethynyl-5-methylpyridin-3-yl)urea P30 (0.186 g, 93%) was concentrated and used in the next stage without purification. LCMS ESI (m/z): 361.5 ([M+H]⁺).

### Preparation 31. Ethyl 7-{4-[5-({[(7-cyclopentylpyrazolo[1,5-a]pyrimidin-6-yl)amino]carbonyl}amino)-3-methylpyridin-2-yl]-1H-1,2,3-triazol-1-yl}heptanoate (P31)

N-(7-Cyclopentylpyrazolo[1,5-a]pyrimidin-6-yl)-N-(6-ethynyl-5-methylpyridin-3-yl)urea P30 (0.186 g, 0.52 mmol), ethyl 7-azidoheptanoate (0.103 g, 0.52 mmol) were dissolved in 20 mL of a solvent consisting of tetrahydrofuran and water in equal amounts. Copper(II) Acetate (0.009 g, 0.05 mmol), and sodium ascorbate (0.01 g, 0.05 mmol) were added into the flask and the mixture was stirred for 12 h at rt. After completion of the reaction as indicated by LCMS, the product was extracted with ethyl acetate and concentrated. The residue was purified on silica gel eluting with ethyl acetate, giving ethyl 7-{4-[5-({[(7-cyclopentylpyrazolo[1,5-a]pyrimidin-6-yl)amino]carbonyl}amino)-3-methylpyridin-2-yl]-1H-1,2,3-triazol-1-yl}heptanoate P31 (0.149 g, 51%). ¹H NMR (400 MHz, DMSO-d₆), δ: 9.12 (s, 1H), 8.53 (s, 1H), 8.47 (s, 1H), 8.46 (s, 1H), 8.42 (s, 1H), 8.23 (d, J=2.4 Hz, 1H), 4.40 (t, J=7.2 Hz, 2H), 4.03 (q, J=7.1 Hz, 2H), 3.87 (s, 1H), 2.60 (s, 3H), 2.33 (s, 2H), 2.26 (t, J=7.3 Hz, 2H), 1.99 (s, 2H), 1.86 (s, 4H), 1.70 (s, 2H), 1.50 (d, J=7.8 Hz, 2H), 1.28 (s, 4H), 1.16 (t, J=7.1 Hz, 3H).

### Preparation 32. 7-{4-[5-({[(7-Cyclopentylpyrazolo[1,5-a]pyrimidin-6-yl)amino]carbonyl}amino)-3-methylpyridin-2-yl]-1H-1,2,3-triazol-1-yl}heptanoic acid (P32)

Aqueous solution of NaOH (0.021 g, 0.53 mmol) was added to a solution of ethyl 7-{4-[5-({[(7-cyclopentylpyrazolo[1,5-a]pyrimidin-6-yl)amino]carbonyl}amino)-3-methylpyridin-2-yl]-1H-1,2,3-triazol-1-yl}heptanoate P31 (0.149 g, 0.27 mmol) in 10 ml of ethanol. The mixture was stirred for 12 h at 50° C. After hydrolysis of the ester was completed, the mixture was cooled down to ambient temperature. Ethanol was evaporated and the reaction mixture was acidified with hydrochloric acid to pH=7, the product was then extracted with ethyl acetate. Solution of the product in ethyl acetate was dried with Na₂SO₄ and evaporated. 7-{4-[5-({[(7-Cyclopentylpyrazolo[1,5-a]pyrimidin-6-yl)amino]carbonyl}amino)-3-methylpyridin-2-yl]-1H-1,2,3-triazol-1-yl}heptanoic acid P32 (0.138 g, 97%) was obtained as a solid. LCMS ESI (m/z): 532.3 ([M+H]⁺).

### Synthesis of 4-(2,7-diazaspiro[3.5]non-2-yl)-6-(2,2,2-trifluoroethyl)pyrido[3,2-d]pyrimidine (P36)

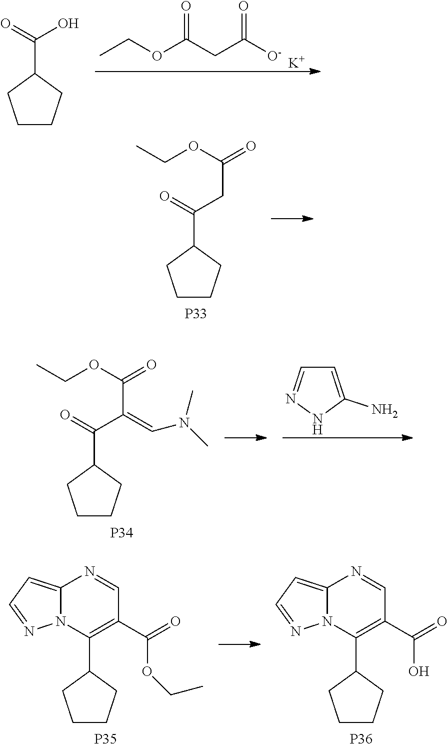

### Preparation 33. Ethyl 3-cyclopentyl-3-oxopropanoate (P33)

1) Carbonyldiimidazole (12.5 g, 77.11 mmol) was added in portions to a mixture of cyclopentanecarboxylic acid (8.0 g, 70.10 mmol) and anhydrous EtOAc (100 mL). The mixture was stirred for 3 h at 50° C.

2) MgCl₂ (26.7 g, 280.4 mmol) was added in portions to a mixture of potassium 3-ethoxy-3-oxo-propanoate (23.86 g, 140.2 mmol) and anhydrous EtOAc (300 mL). The mixture was stirred for 3 h at 50° C.

The solution from step (1) was added dropwise to the suspension from step (2) and the mixture stirred at reflux overnight. The mixture was cooled in an ice-bath and acidified with 4M HCl (140 mL) to pH=3. The mixture was allowed to warm to ambient temperature and the layers were separated. The aqueous layer was extracted with EtOAc, and the combined organic layers were washed with water, brine, and dried (Na₂SO₄). Concentration and purification by chromatography on silica gel gave compound P33 (10.1 g, 78%). ¹H NMR (400 MHz, CDCl₃), δ: 4.18 (q, J=7.1 Hz, 2H), 3.48 (s, 2H), 3.13-2.90 (m, 1H), 2.00-1.70 (m, 4H), 1.72-1.50 (m, 4H), 1.27 (t, J=7.1 Hz, 3H).

### Preparation 34. Ethyl (2Z)-2-(cyclopentylcarbonyl)-3-(dimethylamino)acrylate (P34)

A solution of compound P33 (10.1 g, 54.8 mmol) and N,N-dimethylformamide dimethyl acetal (73 mL, 548.0 mmol) was stirred at reflux overnight. The mixture was evaporated to dryness and the residue was purified by column chromatography on silica gel. Yield of compound P34 (11.0 g, 84%). ¹H NMR (400 MHz, DMSO-d₆), δ: 1H NMR (400 MHz, DMSO-d₆), δ: 7.55 (s, 1H), 4.09 (q, J=7.1 Hz, 2H), 2.83 (br.s, 6H), 1.87-1.37 (m, 9H), 1.21 (t, J=7.1 Hz, 3H).

### Preparation 35. Ethyl 7-cyclopentylpyrazolo[1,5-a]pyrimidine-6-carboxylate (P35)

To a solution of P34 (11.0 g, 46.0 mmol) in 200 mL of EtOH, 1H-pyrazol-5-amine (4.6 g, 55.2 mmol) was added. The mixture was stirred at reflux overnight. The mixture was evaporated, and the residue was purified by column chromatography on silica gel. Yield of compound P35 (11.0 g, 84%). ¹H NMR (400 MHz, CDCl₃), δ: 8.81 (d, J=7.0 Hz, 1H), 8.17 (d, J=2.1 Hz, 1H), 6.70 (d, J=2.0 Hz, 1H), 4.76-4.56 (m, 1H), 4.43 (q, J=7.1 Hz, 2H), 2.61-2.39 (m, 2H), 2.25-2.06 (m, 2H), 1.94 (dt, J=11.4, 8.3 Hz, 2H), 1.80 (dt, J=10.0, 4.5 Hz, 2H), 1.44 (t, J=7.1 Hz, 3H).

### Preparation 36. 7-Cyclopentylpyrazolo[1,5-a]pyrimidine-6-carboxylic acid (P36)

A mixture of compound P35 (9.2 g, 35.5 mmol) and NaOH (2.2 g, 53.2 mmol) in 200 mL EtOH and 50 mL H₂O was stirred at 60° C. for 14 h and evaporated. The residue was diluted with water and acidified with 6M HCl to pH=3-4. The formed solid was filtered off and dried. Yield of compound P36 (8.1 g, 98%). ¹H NMR (400 MHz, DMSO-d₆), δ: 13.62 (s, 1H), 8.78 (d, J=1.1 Hz, 1H), 8.35 (d, J=2.1 Hz, 1H), 6.80 (s, 1H), 4.63 (dd, J=17.8, 9.0 Hz, 1H), 2.39 (br. s, 2H), 2.03 (br. s, 2H), 1.85 (br. s, 2H), 1.72 (br. s, 2H).

### Synthesis of Ethyl 4-[3-(5-amino-3-methylpyridin-2-yl)-1,2,4-oxadiazol-5-yl]butanoate (P39)

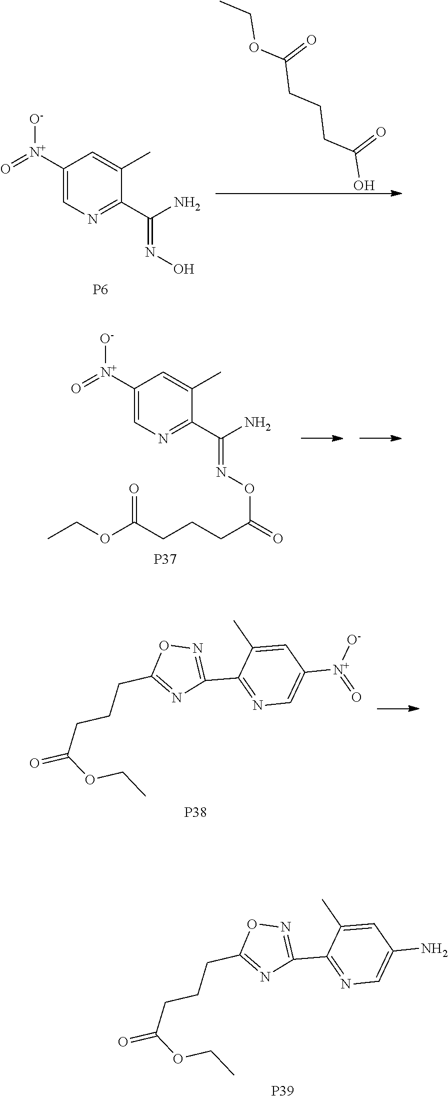

### Preparation 37. Ethyl 5-({[(1Z)-amino(3-methyl-5-nitropyridin-2-yl)methylene]amino}oxy)-5-oxopentanoate (P37)

To a solution of 5-ethoxy-5-oxo-pentanoic acid (0.50 g, 3.1 mmol) in 20 mL of DCM, TEA (1.3 mL, 9.3 mmol) was added. The mixture was stirred at rt for 10 min. Then TBTU (0.94 g, 3.7 mmol) and Compound P6 (0.61 g, 3.1 mmol) were added, and the mixture was stirred for 14 h at ambient temperature. The mixture was washed with 20% K₂CO₃ (aq.), dried with Na₂SO₄ and evaporated. The residue (P37) was used on the next step without additional purification. LCMS ESI (m/z): 339.4 ([M+1]⁺), 197.4, 179.4.

### Preparation 38. Ethyl 4-[3-(3-methyl-5-nitropyridin-2-yl)-1,2,4-oxadiazol-5-yl]butanoate (P38)

A solution of compound P37 in 50 mL of dioxane was stirred at reflux for 96 h. The mixture was evaporated and purified by column chromatography on silica gel. Yield of compound P38 on 2 steps (Preparations 37 and 38) 0.37 g (37%). LCMS ESI (m/z): 321.5 ([M+1]⁺), 275.4, 179.3.

### Preparation 39. Ethyl 4-[3-(5-amino-3-methylpyridin-2-yl)-1,2,4-oxadiazol-5-yl]butanoate (P39)

To a solution of compound P38 (0.37 g, 1.15 mmol) in 20 mL acetic acid, Fe powder (0.65 g, 11.5 mmol) was added. The suspension was stirred at 70° C. for 1 h. The reaction mixture was cooled to ambient temperature, diluted with EtOAc (100 mL), and filtered through Celite. The filtrate was evaporated. The residue was diluted with saturated NaHCO₃ (aq.) and extracted with ethyl acetate. Combined organic extracts were dried with Na₂SO₄ and evaporated. The residue was purified by column chromatography on silica gel. Yield of compound P39 (0.30 g, 89%). LCMS ESI (m/z): 291.4 ([M+1]⁺), 149.6.

### Synthesis of 4-{3-[5-({[(7-cyclopentylpyrazolo[1,5-a]pyrimidin-6-yl)amino]carbonyl}amino)-3-methylpyridin-2-yl]-1,2,4-oxadiazol-5-yl}butanoic acid (P41)

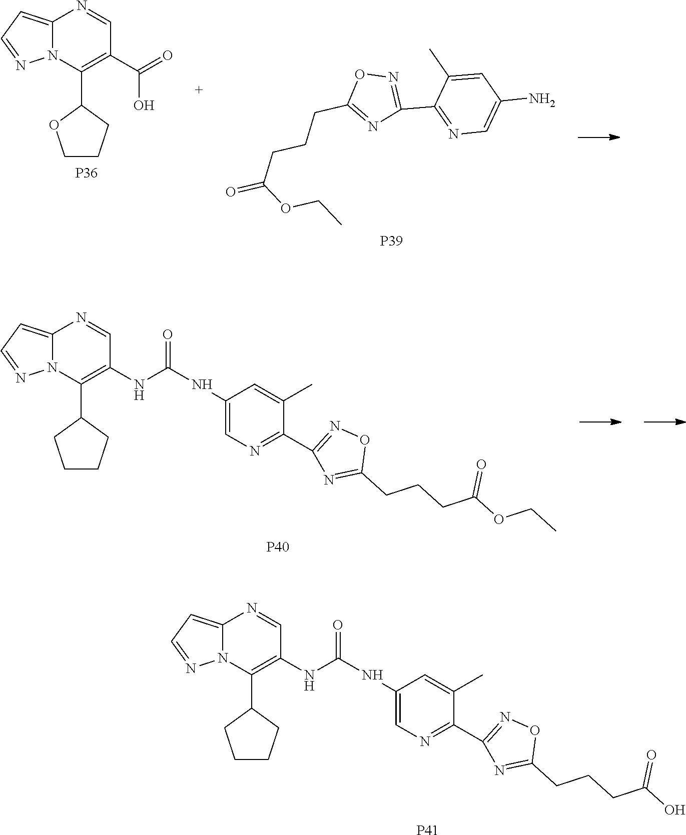

### Preparation 40. Ethyl 4-{3-[5-({[(7-cyclopentylpyrazolo[1,5-a]pyrimidin-6-yl)amino]carbonyl}amino)-3-methylpyridin-2-yl]-1,2,4-oxadiazol-5-yl}butanoate (P40)

To a solution of Compound P36 (0.24 g, 1.0 mmol) in 20 mL of toluene, Et₃N (0.6 mL, 4.0 mmol) was added. The mixture was stirred at rt for 10 min. After this, DPPA (0.27 mL, 1.2 mmol) was added. The mixture was stirred at rt for 1 h, and Compound P39 (0.30 g, 1.0 mmol) was added. The resulting mixture was stirred for 14 h at 90° C. The mixture was evaporated and purified by column chromatography on silica gel to yield the compound P40 (0.35 g, 65%). LCMS ESI (m/z): 519.5 ([M+1]⁺), 431.6.

### Preparation 41. 4-{3-[5-({[(7-cyclopentylpyrazolo[1,5-a]pyrimidin-6-yl)amino]carbonyl}amino)-3-methylpyridin-2-yl]-1,2,4-oxadiazol-5-yl}butanoic acid (P41)

A mixture of compound P40 (0.35 g, 0.7 mmol) and NaOH (0.04 g, 1.05 mmol) in 10 mL EtOH and 2 mL H₂O was stirred at 60° C. for 14 h and evaporated. The residue was diluted with water and acidified with 6M HCl to pH=3-4. The formed solid was filtered off and dried. Yield of compound P41 (0.27 g, 82%). ¹H NMR (400 MHz, DMSO-d₆), δ: 9.42 (s, 1H), 8.63 (s, 1H), 8.56 (s, 1H), 8.48 (s, 1H), 8.23 (t, J=3.7 Hz, 1H), 8.00 (s, 1H), 6.74 (dd, J=6.8, 2.3 Hz, 1H), 4.01-3.76 (m, 1H), 3.04 (t, J=7.4 Hz, 2H), 2.48 (s, 3H), 2.40 (t, J=7.2 Hz, 2H), 2.35 (br.s, 3H), 2.09-1.94 (m, 4H), 1.87 (s, 2H), 1.71 (s, 2H). LCMS ESI (m/z): 491.5 ([M+1]⁺).

### Synthesis of Ethyl 3-[3-(5-amino-3-methylpyridin-2-yl)-1,2,4-oxadiazol-5-yl]propanoate (P44)

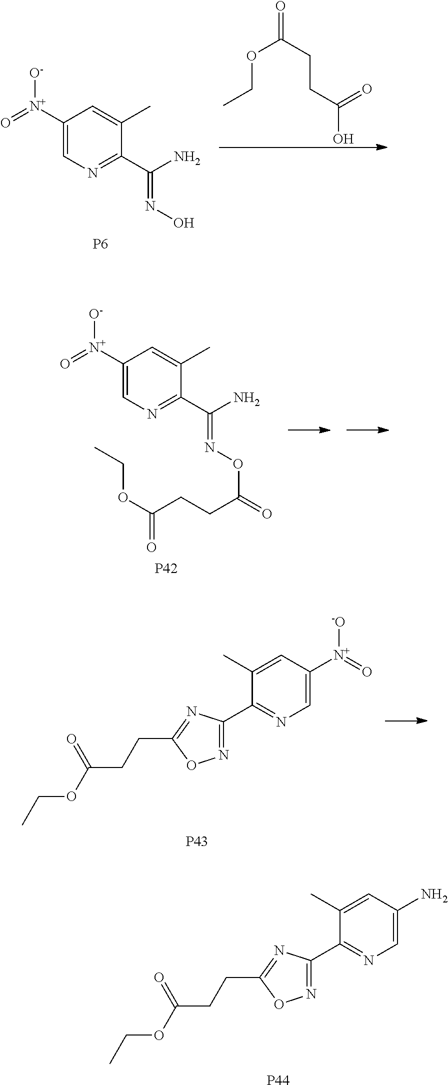

### Preparation 42. Ethyl 4-({[(1Z)-amino(3-methyl-5-nitropyridin-2-yl)methylene]amino}oxy)-4-oxobutanoate (P42)

To a solution of 4-ethoxy-4-oxo-butanoic acid (0.50 g, 3.4 mmol) in 20 mL DCM, Et₃N (1.4 mL, 10.2 mmol) was added. The mixture was stirred at rt for 10 min. After this, TBTU (1.0 g, 4.1 mmol) and Compound P6 (0.67 g, 3.4 mmol) were added, and the mixture was stirred for 14 h at ambient temperature. The mixture was washed with 20% K₂CO₃ (aq.), dried with Na₂SO₄ and evaporated. The residue (P42) was used on the next step without additional purification. LCMS ESI (m/z): 325.5 ([M+1]⁺), 197.4, 179.4.

### Preparation 43. Ethyl 3-[3-(3-methyl-5-nitropyridin-2-yl)-1,2,4-oxadiazol-5-yl]propanoate (P43)

A solution of compound P42 in 50 mL dioxane was stirred at reflux for 96 h. The mixture was evaporated and purified by column chromatography on silica gel. Yield of compound P43 on 2 steps (P42 and P43) 0.56 g (53%). LCMS ESI (m/z): 307.5 ([M+1]⁺), 261.4, 233.1.

### Preparation 44. Ethyl 3-[3-(5-amino-3-methylpyridin-2-yl)-1,2,4-oxadiazol-5-yl]propanoate (P44)

To a solution of compound P43 (0.56 g, 1.8 mmol) in 20 mL of acetic acid, Fe powder (1.0 g, 18.0 mmol) was added. The suspension was stirred at 70° C. for 1 h. The reaction mixture was cooled to ambient temperature, diluted with EtOAc (100 mL) and filtered through Celite. The filtrate was evaporated. The residue was diluted with saturated NaHCO₃ (aq.) and extracted with ethyl acetate. Combined organic extracts were dried with Na₂SO₄ and evaporated. The residue was purified by column chromatography on silica gel. Yield of compound P44 (0.46 g, 91%). LCMS (m/z): 277.4 ([M+1]⁺), 149.4.

### Synthesis of 3-{3-[5-({[(7-Cyclopentylpyrazolo[1,5-a]pyrimidin-6-yl)amino]carbonyl}amino)-3-methylpyridin-2-yl]-1,2,4-oxadiazol-5-yl}propanoic acid

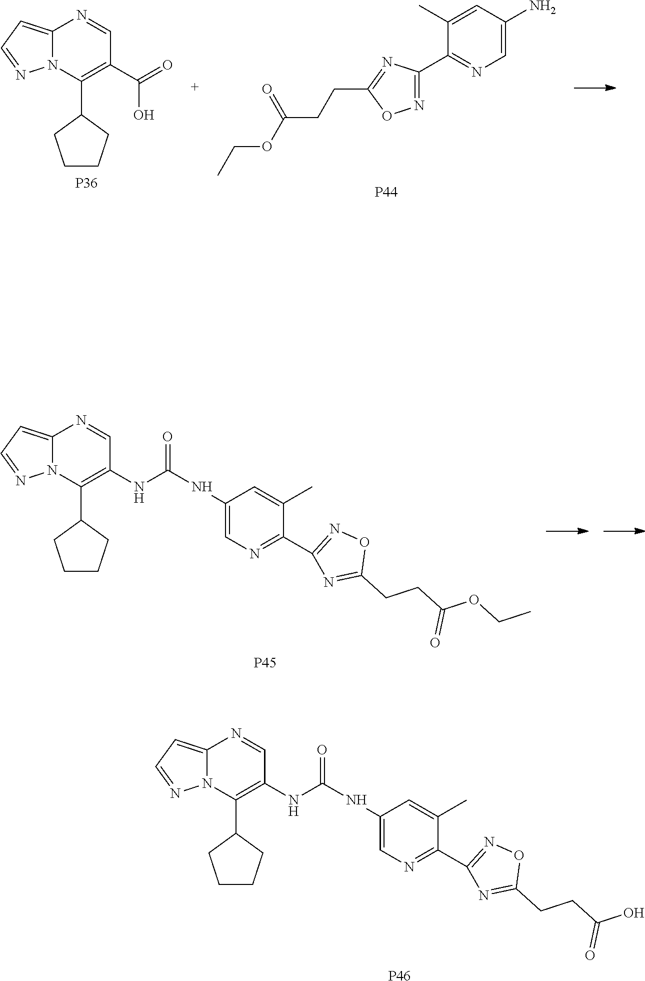

### Preparation 45. Ethyl 3-{3-[5-({[(7-cyclopentylpyrazolo[1,5-a]pyrimidin-6-yl)amino]carbonyl}amino)-3-methylpyridin-2-yl]-1,2,4-oxadiazol-5-yl}propanoate (P45)

To a solution of Compound P36 (0.39 g, 1.7 mmol) in 20 mL of toluene, Et₃N (1.0 mL, 6.8 mmol) was added. The mixture was stirred at rt for 10 min. After this, DPPA (0.44 mL, 2.0 mmol) was added. The mixture was stirred at rt for 1 h, and Compound P44 (0.46 g, 1.7 mmol) was added. The resulting mixture was stirred for 14 h at 90° C. The mixture was evaporated and purified by column chromatography on silica gel. Yield of compound P45 (0.41 g, 48%). LCMS ESI (m/z): 505.5 ([M+1]⁺), 431.6.

### Preparation 46. 3-{3-[5-({[(7-Cyclopentylpyrazolo[1,5-a]pyrimidin-6-yl)amino]carbonyl}amino)-3-methylpyridin-2-yl]-1,2,4-oxadiazol-5-yl}propanoic acid (P46)

A mixture of compound P45 (0.41 g, 0.8 mmol), NaOH (0.05 g, 1.2 mmol) in 10 mL of EtOH and 2 mL of H₂O was stirred at 60° C. for 14 h and evaporated. The residue was diluted with water and acidified with 6M HCl to pH=3-4. The formed solid was filtered off and dried. Yield of compound P46 (0.32 g, 83%). ¹H NMR (400 MHz, DMSO-d₆), δ:12.30 (br. s, 1H), 9.41 (s, 1H), 8.62 (s, 1H), 8.55 (s, 1H), 8.48 (s, 1H), 8.23 (d, J=2.3 Hz, 1H), 8.00 (s, 1H), 6.75 (d, J=2.3 Hz, 1H), 3.98-3.81 (m, 1H), 3.19 (t, J=6.8 Hz, 2H), 2.84 (t, J=6.8 Hz, 2H), 2.46 (s, 3H), 2.35 (s, 2H), 1.99 (s, 2H), 1.86 (s, 2H), 1.71 (s, 2H). LCMS ESI (m/z): 477.3 ([M+1]⁺).

### Synthesis of Ethyl 5-[3-(5-amino-3-methylpyridin-2-yl)-1,2,4-oxadiazol-5-yl]pentanoate (P49)

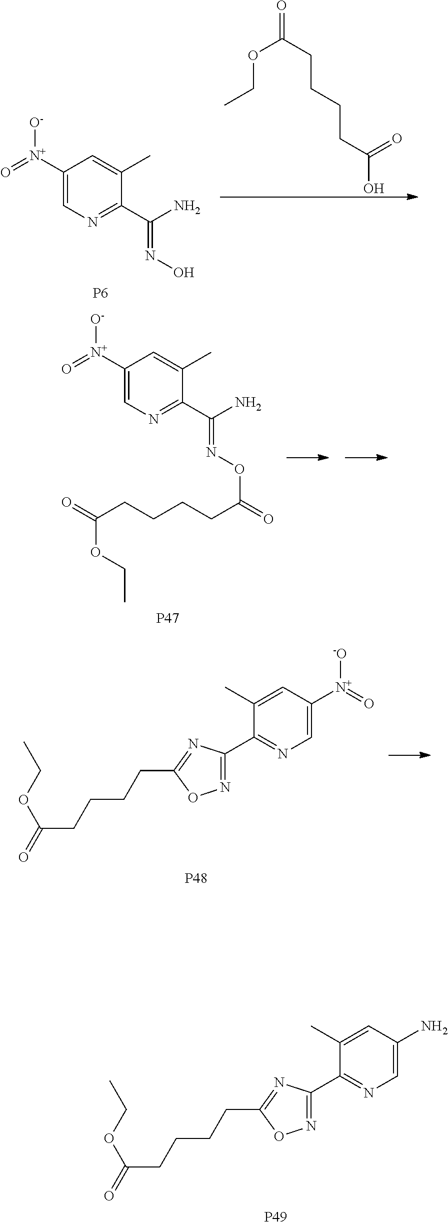

### Preparation 47. Ethyl 6-({[(1Z)-amino(3-methyl-5-nitropyridin-2-yl)methylene]amino}oxy)-6-oxohexanoate (P47)

To a solution of 6-ethoxy-6-oxo-hexanoic acid (0.60 g, 3.4 mmol) in 20 mL of DCM, Et₃N (1.5 mL, 10.2 mmol) was added. The mixture was stirred at rt for 10 min. Then TBTU (1.03 g, 4.1 mmol) and P6 (0.67 g, 3.4 mmol) were added, and the mixture was stirred for 14 h at ambient temperature. The mixture was washed with 20% K₂CO₃ (aq.), dried with Na₂SO₄ and evaporated. The residue was used on the next step without additional purification. LCMS (m/z): 353.4 ([M+1]⁺), 197.3, 179.3.

### Preparation 48. Ethyl 5-[3-(3-methyl-5-nitropyridin-2-yl)-1,2,4-oxadiazol-5-yl]pentanoate (P48)

A solution of compound P47 in 50 mL of dioxane was stirred at reflux for 96 h. The mixture was evaporated and purified by column chromatography on silica gel. Yield of compound P48 on 2 steps (P47 and P48) 0.58 g (51%). LCMS ESI (m/z): 335.5 ([M+1]⁺), 289.3.

### Preparation 49. Ethyl 5-[3-(5-amino-3-methylpyridin-2-yl)-1,2,4-oxadiazol-5-yl]pentanoate (P49)

To a solution of compound P48 (0.58 g, 1.7 mmol) in 20 mL of acetic acid, Fe powder (1.0 g, 17 mmol) was added. The suspension was stirred at 70° C. for 1 h. The reaction mixture was cooled to ambient temperature, diluted with EtOAc (100 mL) and filtered through Celite. The filtrate was evaporated. The residue was diluted with saturated NaHCO₃ (aq.) and extracted with ethyl acetate. Combined organic extracts were dried with Na₂SO₄ and evaporated. The residue was purified by column chromatography on silica gel. Yield of compound P49 (0.47 g, 84%). LCMS ESI (m/z): 305.5 ([M+1]⁺), 149.4.

### Synthesis of 5-Formyl-4-methyl-1H-indole-2-carbonitril N-(7-Cyclopentylpyrazolo[1,5-a]pyrimidin-6-yl)-N′-{5-methyl-6-[5-(5-oxopentyl)-1,2,4-oxadiazol-3-yl]pyridin-3-yl}urea (P53)

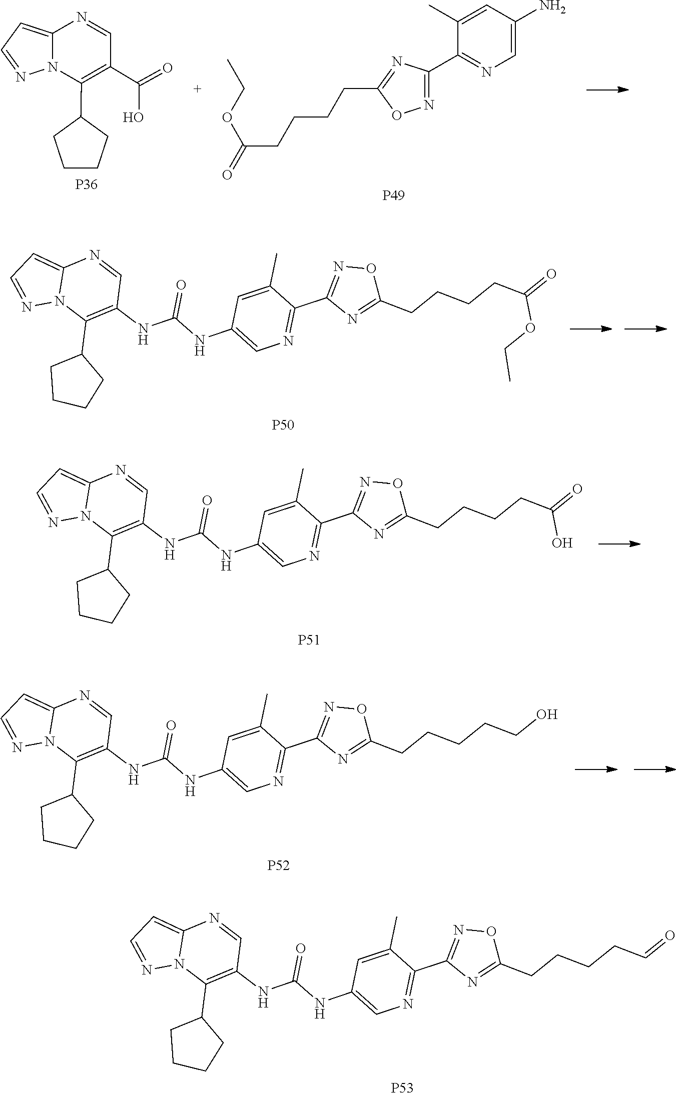

### Preparation 50. Ethyl 5-{3-[5-({[(7-cyclopentylpyrazolo[1,5-a]pyrimidin-6-yl)amino]carbonyl}amino)-3-methylpyridin-2-yl]-1,2,4-oxadiazol-5-yl}pentanoate (P50)

To a solution of Compound P36 (0.36 g, 1.6 mmol) in 20 mL of toluene, Et₃N (0.9 mL, 6.4 mmol) was added. The mixture was stirred at rt for 10 min. Then DPPA (0.4 mL, 2.0 mmol) was added. The mixture was stirred at rt for 1 h, and Compound P49 (0.47 g, 1.6 mmol) was added. The resulting mixture was stirred for 14 h at 90° C. The mixture was evaporated and purified by column chromatography on silica gel. Yield of compound P50 (0.52 g, 63%). LCMS ESI (m/z): 533.5 ([M+1]⁺), 377.5.

### Preparation 51. 5-{3-[5-({[(7-Cyclopentylpyrazolo[1,5-a]pyrimidin-6-yl)amino]carbonyl}amino)-3-methylpyridin-2-yl]-1,2,4-oxadiazol-5-yl}pentanoic acid (P51)

A mixture of compound P50 (0.52 g, 1.0 mmol), NaOH (0.06 g, 1.5 mmol) in 10 mL EtOH and 2 mL H₂O was stirred at 60° C. for 14 h and evaporated. The residue was diluted with water and acidified with 6M HCl to pH=3-4. The formed solid was filtered off and dried. Yield of compound P51 (0.45 g, 92%). LCMS (m/z): 505.5 ([M+1]⁺).

### Preparation 52. N-(7-Cyclopentylpyrazolo[1,5-a]pyrimidin-6-yl)-N′-{6-[5-(5-hydroxypentyl)-1,2,4-oxadiazol-3-yl]-5-methylpyridin-3-yl}urea (P52)

To a solution of Compound P51 (0.45 g, 0.9 mmol) in 10 mL of THF, Et₃N (0.16 mL, 1.17 mmol) was added. The mixture was stirred at rt for 10 min. After this, ethyl chloroformate (0.11 mL, 1.17 mmol) was added at 0-5° C. The mixture was stirred at rt for 1 h. The formed solid was filtered off and the filtrate was added dropwise to a solution of sodium borohydride (0.17 g, 4.5 mmol) in water (10 mL) at 0-5° C. The resulting mixture was stirred for 24 h at ambient temperature. The mixture was diluted with water and extracted with ethyl acetate. Combined organic extracts were dried with Na₂SO₄ and evaporated. The residue purified by column chromatography on silica gel. Yield of compound P52 (0.34 g, 78%). LCMS (m/z): 491.4 ([M+1]⁺).

### Preparation 53. N-(7-Cyclopentylpyrazolo[1,5-a]pyrimidin-6-yl)-N′-{5-methyl-6-[5-(5-oxopentyl)-1,2,4-oxadiazol-3-yl]pyridin-3-yl}urea (P53)

A solution of dimethyl sulfoxide (0.14 mL, 1.88 mmol) in DCM (0.1 mL) was added to a stirred solution of trifluoroacetic anhydride (0.2 mL, 1.37 mmol) in DCM (10 mL) at −70° C. A solution of Compound P52 (0.28 g, 0.57 mmol) in DCM (1.0 mL) was added dropwise to the above prepared solution over a period of 15 min and the mixture was stirred for an additional 3.5 h. Triethylamine (0.5 mL, 3.42 mmol) was added dropwise with stirring for 15 min, and the whole was allowed to warm to rt. The mixture was diluted with DCM, washed saturated NaHCO₃ (aq.), dried with Na₂SO₄ and evaporated. The residue (P53) was used on the next step without additional purification. LCMS ESI (m/z): 489.4 ([M+1]⁺).

### Synthesis of N-(7-Cyclopentylpyrazolo[1,5-a]pyrimidin-6-yl)-N′-{5-methyl-6-[5-(3-oxopropyl)-1,2,4-oxadiazol-3-yl]pyridin-3-yl}urea (P55)

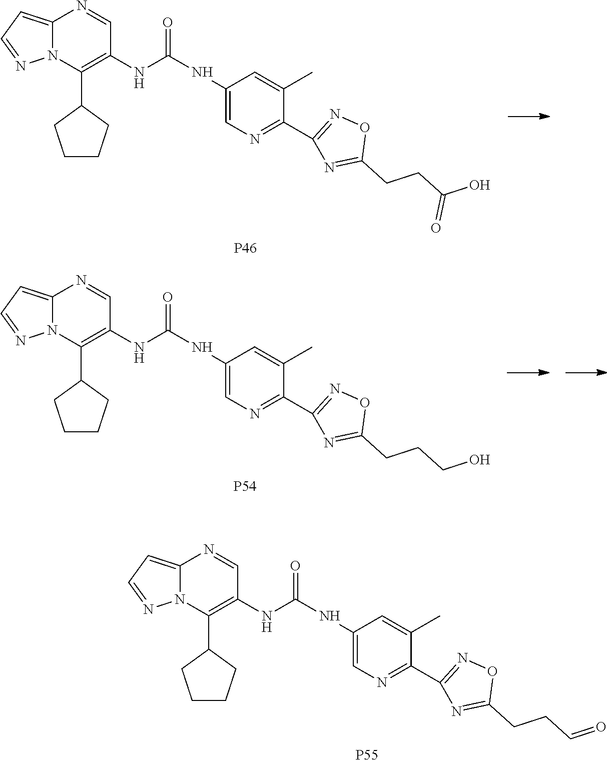

### Preparation 54. N-(7-Cyclopentylpyrazolo[1,5-a]pyrimidin-6-yl)-N′-{6-[5-(3-hydroxypropyl)-1,2,4-oxadiazol-3-yl]-5-methylpyridin-3-yl}urea (P54)

To a solution of Compound P46 (0.4 g, 0.8 mmol) in 10 mL of THF, Et₃N (0.16 mL, 1.04 mmol) was added. The mixture was stirred at rt for 10 min. After this, ethyl chloroformate (0.1 mL, 1.04 mmol) was added at 0-5° C. The mixture was stirred at rt for 1 h. The formed solid was filtered off and the filtrate was added dropwise to a solution of sodium borohydride (0.16 g, 4.0 mmol) in water (10 mL) at 0-5° C. The resulting mixture was stirred for 24 h at ambient temperature. The mixture was diluted with water and extracted with ethyl acetate. Combined organic extracts were dried with Na₂SO₄ and evaporated. The residue purified by column chromatography on silica gel to yield the compound P54 (0.22 g, 57%). ¹H NMR (400 MHz, DMSO-d₆), δ: 9.39 (s, 1H), 8.62 (d, J=2.1 Hz, 1H), 8.54 (s, 1H), 8.48 (s, 1H), 8.23 (d, J=2.2 Hz, 1H), 7.99 (s, 1H), 6.75 (d, J=2.2 Hz, 1H), 4.63 (br. s, 1H), 3.86 (dd, J=18.0, 9.1 Hz, 1H), 3.51 (t, J=6.1 Hz, 2H), 3.03 (t, J=7.5 Hz, 2H), 2.47 (s, 3H), 2.35 (s, 2H), 2.11-1.78 (m, 6H), 1.70 (d, J=5.1 Hz, 2H). LCMS ESI (m/z): 463.7 ([M+1]⁺).

### Preparation 55. N-(7-Cyclopentylpyrazolo[1,5-a]pyrimidin-6-yl)-N′-{5-methyl-6-[5-(3-oxopropyl)-1,2,4-oxadiazol-3-yl]pyridin-3-yl}urea (P55)

A solution of DMSO (0.03 mL, 0.43 mmol) in DCM (0.1 mL) was added to a stirred solution of trifluoroacetic anhydride (0.045 mL, 0.31 mmol) in DCM (10 mL) at −70° C. A solution of Compound P54 (0.06 g, 0.13 mmol) in DCM (1.0 mL) was added dropwise to the above prepared solution over a period of 15 min and the mixture was stirred for an additional 3.5 h. Triethylamine (0.11 mL, 0.78 mmol) was added dropwise with stirring for 15 min, and the whole was allowed to warm to room temperature. The mixture was diluted with DCM, washed saturated NaHCO₃ (aq.), dried with Na₂SO₄ and evaporated. The residue (P55) was used on the next step without additional purification. LCMS ESI (m/z): 461.4 ([M+1]⁺).

### Synthesis of N-(3-Chloro-7-cyclopentylpyrazolo[1,5-a]pyrimidin-6-yl)-N′-{5-methyl-6-[5-(3-oxopropyl)-1,2,4-oxadiazol-3-yl]pyridin-3-yl}urea (P56)

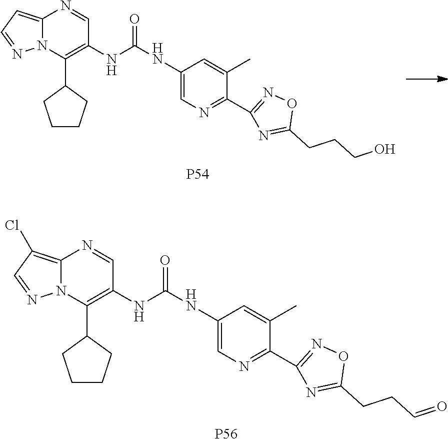

### Preparation 56. N-(3-Chloro-7-cyclopentylpyrazolo[1,5-a]pyrimidin-6-yl)-N′-{5-methyl-6-[5-(3-oxopropyl)-1,2,4-oxadiazol-3-yl]pyridin-3-yl}urea (P56)

A solution of DMSO (0.06 mL, 0.80 mmol) in DCM (0.1 mL) was added to a stirred solution of oxalyl chloride (0.05 mL, 0.53 mmol) in DCM (10 mL) at −70° C. A solution of Compound P54 (0.1 g, 0.24 mmol) in DCM (1.0 mL) was added dropwise to the above prepared solution over a period of 15 min and the mixture was stirred for an additional 3.5 h. Triethylamine (0.20 mL, 1.44 mmol) was added dropwise with stirring for 15 min, and the whole was allowed to warm to room temperature. The mixture was diluted with CH₂Cl₂, washed saturated NaHCO₃ (aq.), dried with Na₂SO₄ and evaporated. The residue was used on the next step without additional purification. LCMS ESI (m/z): 495.4 ([M+1]⁺).

### Synthesis of N-(3-chloro-7-cyclopentylpyrazolo[1,5-a]pyrimidin-6-yl)-N′-{5-methyl-6-[5-(4-oxobutyl)-1,2,4-oxadiazol-3-yl]pyridin-3-yl}urea (P58)

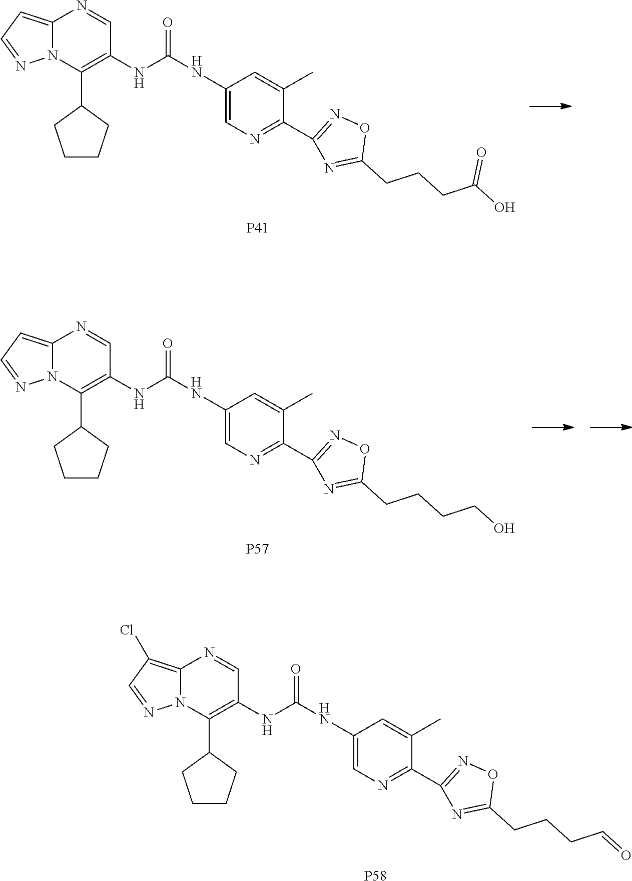

### Preparation 57. N-(7-cyclopentylpyrazolo[1,5-a]pyrimidin-6-yl)-N′-{6-[5-(4-hydroxybutyl)-1,2,4-oxadiazol-3-yl]-5-methylpyridin-3-yl}urea (P57)

To a solution of Compound P41 (0.27 g, 0.55 mmol) in 10 mL of THF, Et₃N (0.1 mL, 0.72 mmol) was added. The mixture was stirred at rt for 10 min. Then, ethyl chloroformate (0.07 mL, 0.72 mmol) was added at 0-5° C. The mixture was stirred at rt for 1 h. Formed solid was filtered off and the filtrate was added dropwise to a solution of sodium borohydride (0.1 g, 2.75 mmol) in water (10 mL) at 0-5° C. The resulting mixture was stirred for 24 h at ambient temperature. The mixture was diluted with water and extracted with ethyl acetate. Combined organic extracts were dried with Na₂SO₄ and evaporated. The residue purified by column chromatography on silica gel. Yield of compound P57: 0.20 g, 76%. LCMS ESI (m/z): 477.6 ([M+1]⁺), 377.4.

### Preparation 58. N-(3-chloro-7-cyclopentylpyrazolo[1,5-a]pyrimidin-6-yl)-N′-{5-methyl-6-[5-(4-oxobutyl)-1,2,4-oxadiazol-3-yl]pyridin-3-yl}urea (P58)

A solution of DMSO (0.05 mL, 0.66 mmol) in DCM (0.1 mL) was added to a stirred solution of oxalyl chloride (0.045 mL, 0.48 mmol) in DCM (10 mL) at −70° C. A solution of Compound P57 (0.1 g, 0.2 mmol) in DCM (1.0 mL) was added dropwise to the above prepared solution over a period of 15 min and the mixture was stirred for an additional 3.5 h. Triethylamine (0.2 mL, 1.20 mmol) was added dropwise with stirring for 15 min, and the whole was allowed to warm to rt. The mixture was diluted with DCM, washed saturated NaHCO₃ (aq.), dried with Na₂SO₄ and evaporated. The residue (P58) was used on the next step without additional purification. LCMS ESI (m/z): 509.4 ([M+1]⁺).

### Synthesis of N-(7-Cyclopentylpyrazolo[1,5-a]pyrimidin-6-yl)-N′-{5-methyl-6-[5-(4-oxobutyl)-1,2,4-oxadiazol-3-yl]pyridin-3-yl}urea (P59)

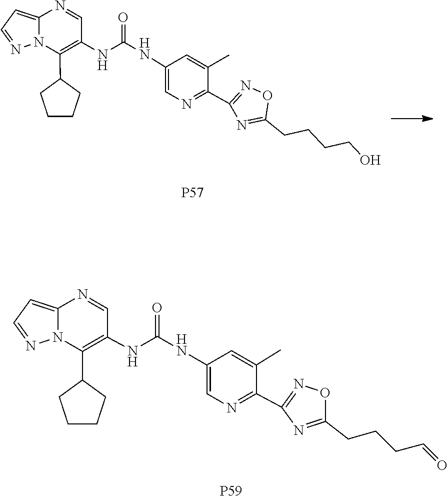

### Preparation 59. N-(7-Cyclopentylpyrazolo[1,5-a]pyrimidin-6-yl)-N′-{5-methyl-6-[5-(4-oxobutyl)-1,2,4-oxadiazol-3-yl]pyridin-3-yl}urea (P59)

A solution of DMSO (0.025 mL, 0.33 mmol) in DCM (0.1 mL) was added to a stirred solution of trifluoroacetic anhydride (0.036 mL, 0.24 mmol) in DCM (10 mL) at −70° C. A solution of Compound P57 (0.05 g, 0.1 mmol) in DCM (1.0 mL) was added dropwise to the above prepared solution over a period of 15 min and the mixture was stirred for an additional 3.5 h. Triethylamine (0.09 mL, 0.60 mmol) was added dropwise with stirring for 15 min, and the whole was allowed to warm to rt. The mixture was diluted with DCM, washed saturated NaHCO₃ (aq.), dried with Na₂SO₄ and evaporated. The residue (P59) was used on the next step without additional purification. LCMS ESI (m/z): 475.4 ([M+1]⁺).

### Synthesis of Ethyl 6-[3-(3-methyl-5-nitropyridin-2-yl)-1,2,4-oxadiazol-5-yl]hexanoate (P8)

### The Second Option

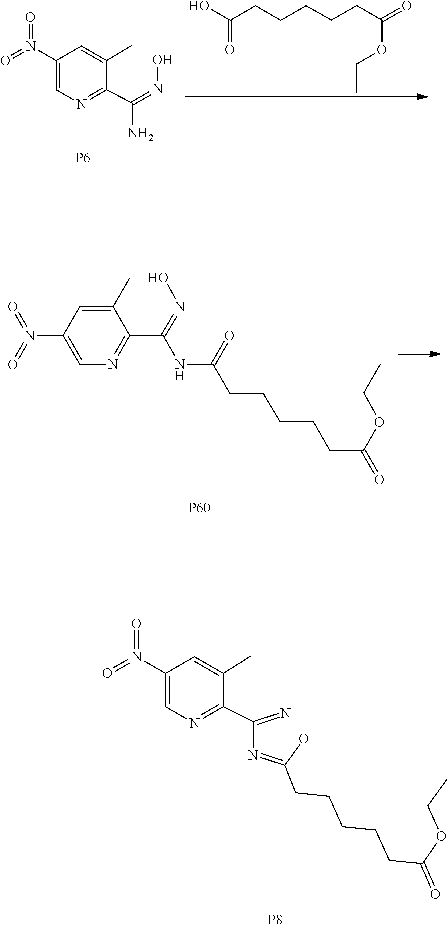

### Preparation 60. Ethyl 7-{[(E)-(hydroxyimino)(3-methyl-5-nitropyridin-2-yl)methyl]amino}-7-oxoheptanoate (P60)

To a solution of 7-ethoxy-7-oxoheptanoic acid (0.78 g, 4.2 mmol) in 40 mL of 1,4-dioxane, N-ethyl-N-isopropylpropan-2-amine (0.645 g, 4.98 mmol) was added. The solution was stirred for 5 min, then N-hydroxy-3-methyl-5-nitropyridine-2-carboximidamide P6 (0.816 g, 4.2 mmol) was added, and the mixture was stirred for 10 min. 2,4,6-Tripropyl-1,3,5,2,4,6-trioxatriphosphinane 2,4,6-trioxide (1.98 g, 6.3 mmol, 50 wt % solution in EtOAc) was poured into the reaction flask and the mixture was stirred for 12 h at rt. After completion of the reaction as indicated by TLC, the reaction mixture was concentrated under vacuum, dissolved in ethyl acetate, and washed with water and evaporated. The residue (P60) was used for the next step without additional purification. LCMS (m/z): 366.3 ([M+1]⁺).

### Preparation 8a. Ethyl 6-[3-(3-methyl-5-nitropyridin-2-yl)-1,2,4-oxadiazol-5-yl]hexanoate (P8)

Ethyl 7-{[(E)-(hydroxyimino)(3-methyl-5-nitropyridin-2-yl)methyl]amino}-7-oxoheptanoate P60 (0.815 g, 4.16 mmol) was dissolved in 50 mL of 1,4-dioxane. The solution was refluxed for 96 h. After completion of the reaction as indicated by TLC, the reaction mixture was concentrated under vacuum. The residue was purified on silica gel eluting with EtOAc/hexane, 1:10, giving ethyl 6-[3-(3-methyl-5-nitropyridin-2-yl)-1,2,4-oxadiazol-5-yl]hexanoate P8 (0.55 g). The yield was 38% for two steps (Preparation 60 and Preparation 8a). ¹H NMR (400 MHz, DMSO-d₆), δ: 9.34 (d, J=2.4 Hz, 1H), 8.72 (d, J=2.5 Hz, 1H), 4.04 (q, J=7.1 Hz, 2H), 3.06 (t, J=7.5 Hz, 2H), 2.64 (s, 3H), 2.30 (t, J=7.3 Hz, 2H), 1.90-1.71 (m, 2H), 1.67-1.52 (m, 2H), 1.50-1.29 (m, 2H), 1.16 (t, J=7.1 Hz, 3H).

### Synthesis of 6-{3-[5-({[(7-Cyclopentylpyrazolo[1,5-a]pyrimidin-6-yl)amino]carbonyl}amino)-3-methylpyridin-2-yl]-1,2,4-oxadiazol-5-yl}hexanoic acid (P62)

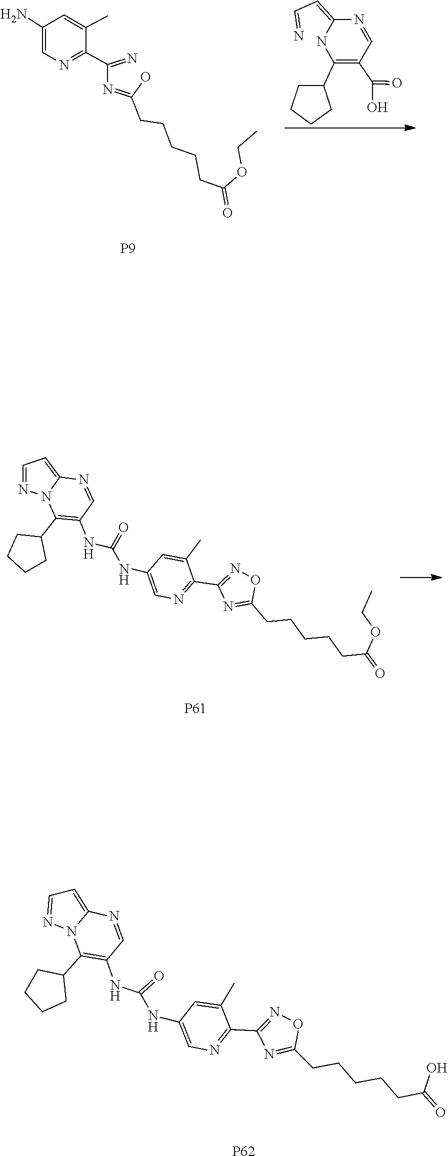

### Preparation 61. Ethyl 6-{3-[5-({[(7-cyclopentylpyrazolo[1,5-a]pyrimidin-6-yl)amino]carbonyl}amino)-3-methylpyridin-2-yl]-1,2,4-oxadiazol-5-yl}hexanoate (P61)

Triethylamine (0.166 g, 1.64 mmol) was added to a suspension of 7-cyclopentylpyrazolo[1,5-a]pyrimidine-6-carboxylic acid (0.095 g, 0.41 mmol) in 40 mL of toluene. The mixture was stirred for 5 min. Then DPPA (1.136 g, 0.49 mmol) was added and stirred for 60 min at rt. After this ethyl 6-[3-(5-amino-3-methylpyridin-2-yl)-1,2,4-oxadiazol-5-yl]hexanoate P9 (0.13 g, 0.41 mmol) was added, and the mixture was refluxed for 12 h. Reaction was monitored by LCMS. The mixture was quenched with water, separated toluene was dried with Na₂SO₄ and evaporated. The residue was purified on silica gel eluting with hexane/ethyl acetate, 1:1, giving ethyl 6-{3-[5-({[(7-cyclopentylpyrazolo[1,5-a]pyrimidin-6-yl)amino]carbonyl}amino)-3-methylpyridin-2-yl]-1,2,4-oxadiazol-5-yl}hexanoate P61 (0.129 g, 57%). ¹H NMR (400 MHz, DMSO-d₆), δ: 9.35 (s, 1H), 8.62 (d, J=2.2 Hz, 1H), 8.51 (s, 1H), 8.48 (s, 1H), 8.23 (d, J=2.3 Hz, 1H), 8.00 (s, 1H), 6.75 (d, J=2.3 Hz, 1H), 4.04 (q, J=7.1 Hz, 2H), 3.94-3.78 (m, 1H), 3.00 (t, J=7.5 Hz, 2H), 2.47 (s, 3H), 2.34 (d, J=11.0 Hz, 2H), 2.30 (t, J=7.3 Hz, 2H), 1.99 (s, 2H), 1.86 (s, 2H), 1.78 (dd, J=15.4, 7.5 Hz, 2H), 1.71 (s, 2H), 1.57 (dd, J=15.3, 7.3 Hz, 2H), 1.44-1.33 (m, 2H), 1.16 (t, J=7.1 Hz, 3H).

### Preparation 62. 6-{3-[5-({[(7-Cyclopentylpyrazolo[1,5-a]pyrimidin-6-yl)amino]carbonyl}amino)-3-methylpyridin-2-yl]-1,2,4-oxadiazol-5-yl}hexanoic acid (P62)

An aqueous solution of NaOH (0.026 g, 0.46 mmol) was added to a solution of ethyl 6-{3-[5-({[(7-cyclopentylpyrazolo[1,5-a]pyrimidin-6-yl)amino]carbonyl}amino)-3-methylpyridin-2-yl]-1,2,4-oxadiazol-5-yl}hexanoate P61 (0.127 g, 0.23 mmol) in 10 ml of ethanol. The mixture was stirred for 12 h at 50° C. After the full hydrolysis of the ester, the mixture was cooled down to ambient temperature. Ethanol was evaporated and the reaction mixture was acidified with hydrochloric acid to pH=7, the product was then extracted with ethyl acetate. Solution of the product in ethyl acetate was dried with Na₂SO₄ and evaporated. 6-{3-[5-({[(7-Cyclopentylpyrazolo[1,5-a]pyrimidin-6-yl)amino]carbonyl}amino)-3-methylpyridin-2-yl]-1,2,4-oxadiazol-5-yl}hexanoic acid P62 (0.102 g, 84%) was obtained as a solid. ¹H NMR (400 MHz, DMSO-d₆), δ: 11.99 (s, 1H), 9.36 (s, 1H), 8.62 (d, J=2.2 Hz, 1H), 8.51 (s, 1H), 8.48 (s, 1H), 8.23 (d, J=2.3 Hz, 1H), 7.99 (s, 1H), 6.75 (d, J=2.3 Hz, 1H), 4.03 (q, J=7.1 Hz, 1H), 3.94-3.77 (m, 1H), 3.00 (t, J=7.4 Hz, 2H), 2.47 (s, 3H), 2.34 (d, J=8.5 Hz, 2H), 2.22 (t, J=7.3 Hz, 2H), 1.99 (s, 2H), 1.92-1.83 (m, 2H), 1.78 (dd, J=15.1, 7.5 Hz, 2H), 1.71 (s, 2H), 1.54 (dd, J=15.1, 7.4 Hz, 2H), 1.39 (dd, J=14.8, 8.0 Hz, 2H).

### Synthesis of tert-Butyl (4-azidobutoxy)acetate (P64)

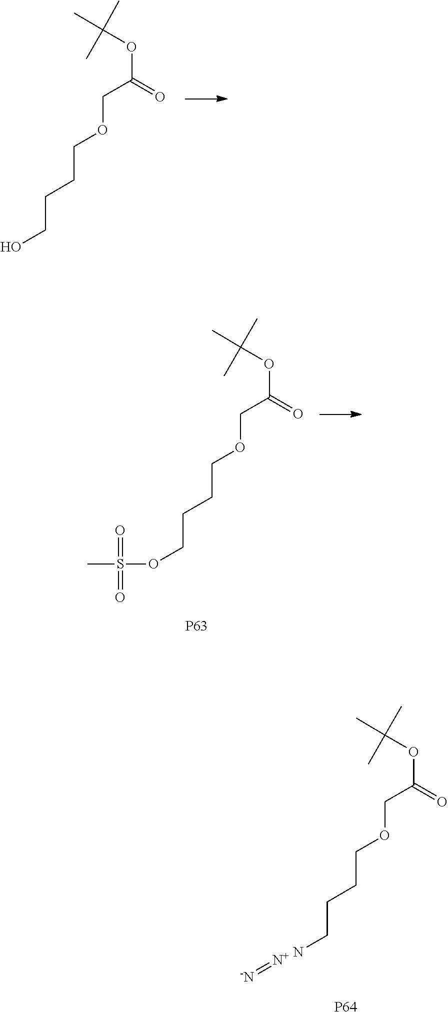

### Preparation 63. tert-Butyl {4-[(methylsulfonyl)oxy]butoxy}acetate (P63)

Methanesulfonyl chloride (0.673 g, 5.87 mmol) was added to a solution of tert-butyl (4-hydroxybutoxy)acetate (1.0 g, 4.9 mmol) and triethylamine (0.743 g, 7.4 mmol) in dry DCM (30 mL) at 0° C. The mixture was stirred overnight, then washed with water, 1 M hydrochloric acid, 5% aqueous solution of sodium bicarbonate, and brine. The organic phase was dried over anhydrous sodium sulfate, filtered, and evaporated, giving tert-butyl {4-[(methylsulfonyl)oxy]butoxy}acetate P63 (1.0 g, 72%). ¹H NMR (400 MHz, DMSO-d₆), δ: 4.22 (t, J=6.4 Hz, 2H), 3.93 (d, J=9.5 Hz, 2H), 3.46 (t, J=6.2 Hz, 2H), 3.16 (d, J=7.3 Hz, 3H), 1.82-1.67 (m, 2H), 1.59 (dt, J=12.7, 6.3 Hz, 2H), 1.42 (s, 9H).

### Preparation 64. tert-Butyl (4-azidobutoxy)acetate (P64)

Sodium azide (1.15 g, 17.5 mmol) was added to the solution of the tert-butyl {4-[(methylsulfonyl)oxy]butoxy}acetate in DMF (30 ml). The mixture was stirred with heating 80° C. for 18 hours. When reaction was completed (TLC monitoring), the reaction mixture was cooled to room temperature, diluted with ethyl acetate, and washed with water. The product was purified by column chromatography on silica gel. Eluent—hexane/ethyl acetate, 40:1. tert-Butyl (4-azidobutoxy)acetate (0.395 g) was obtained with a yield of 48%. ¹H NMR (400 MHz, DMSO-d₆), δ: 3.94 (s, 2H), 3.45 (t, J=5.9 Hz, 2H), 3.35 (t, J=6.6 Hz, 2H), 1.68-1.49 (m, 4H), 1.42 (s, 9H).

### Synthesis of (4-{4-[5-({[(7-Cyclopentylpyrazolo[1,5-a]pyrimidin-6-yl)amino]carbonyl}amino)-3-methylpyridin-2-yl]-1H-1,2,3-triazol-1-yl}butoxy)acetic acid (P66)

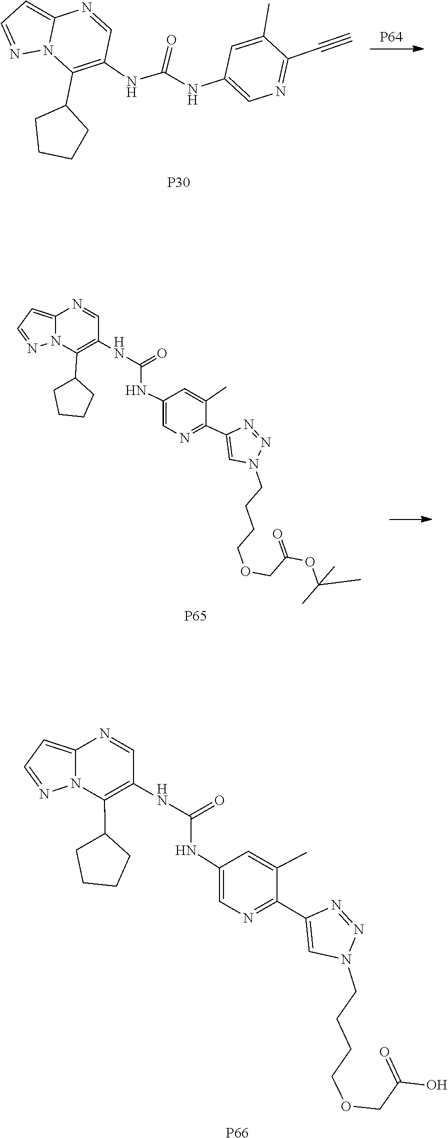

### Preparation 65. tert-Butyl (4-{4-[5-({[(7-cyclopentylpyrazolo[1,5-a]pyrimidin-6-yl)amino]carbonyl}amino)-3-methylpyridin-2-yl]-1H-1,2,3-triazol-1-yl}butoxy)acetate (P65)

N-(7-Cyclopentylpyrazolo[1,5-a]pyrimidin-6-yl)-N′-(6-ethynyl-5-methylpyridin-3-yl)urea P30 (0.232 g, 0.64 mmol), tert-butyl (4-azidobutoxy)acetate (0.147 g, 0.64 mmol) were dissolved in 20 mL of a solvent consisting of tetrahydrofuran and water in equal amounts. Copper(II) Acetate (0.012 g, 0.06 mmol) and sodium ascorbate (0.013 g, 0.06 mmol) were added into the flask and the mixture was stirred for 12 h at rt. After completion of the reaction as indicated by LCMS, the product was extracted with ethyl acetate and concentrated. The residue was purified on silica gel eluting with EtOAc/MeOH, 40:1, giving tert-butyl (4-{4-[5-({[(7-cyclopentylpyrazolo[1,5-a]pyrimidin-6-yl)amino]carbonyl}amino)-3-methylpyridin-2-yl]-1H-1,2,3-triazol-1-yl}butoxy)acetate P65 (0.157 g, 41%). LCMS ESI (m/z): 590.5 ([M+1]⁺), 534.3.

### Preparation 66. (4-{4-[5-({[(7-Cyclopentylpyrazolo[1,5-a]pyrimidin-6-yl)amino]carbonyl}amino)-3-methylpyridin-2-yl]-1H-1,2,3-triazol-1-yl}butoxy)acetic acid (P66)

tert-Butyl (4-{4-[5-({[(7-cyclopentylpyrazolo[1,5-a]pyrimidin-6-yl)amino]carbonyl}amino)-3-methylpyridin-2-yl]-1H-1,2,3-triazol-1-yl}butoxy)acetate P65 (0.157 g, 0.27 mmol) was dissolved in 10 ml DCM with TFA (0.303 g, 2.66 mmol). The solution was stirred at rt for 15 h. TLC control. The product was concentrated using a vacuum and was evaporated with dioxane saturated with HCl. (4-{4-[5-({[(7-cyclopentylpyrazolo[1,5-a]pyrimidin-6-yl)amino]carbonyl}amino)-3-methylpyridin-2-yl]-1H-1,2,3-triazol-1-yl}butoxy)acetic acid hydrochloride P66 (0.15 g) was obtained with a yield of 98%. LCMS ESI (m/z): 534.5 ([M+1]⁺).

### Synthesis of [4-(Benzoyloxy)butoxy]acetic acid (P68)

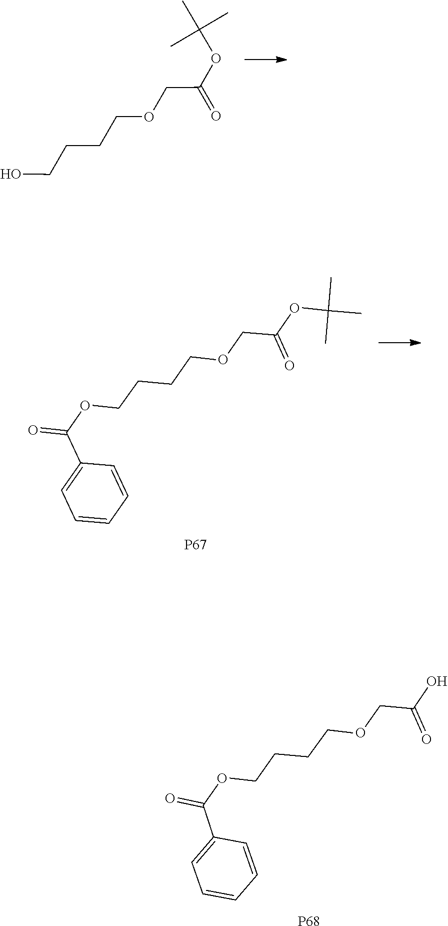 [SMILES: CC(C)(C)OC(=O)COCCCCO ; "P67" ; CC(C)(C)OC(=O)COCCCCOC(=O)c1ccccc1 ; "P68" ; O=C(O)COCCCCOC(=O)c1ccccc1]

### Preparation 67. 4-(2-Tert-butoxy-2-oxoethoxy)butyl benzoate (P67)

To a solution of tert-butyl 2-(4-hydroxybutoxy)acetate (1.0 g, 4.9 mmol) in 20 mL of DCM, Et₃N (1.7 mL, 12.3 mmol) was added. Then, benzoyl chloride (0.7 mL, 5.9 mmol) was added at 0-5° C. The mixture was stirred at rt for 14 h. The mixture was diluted with DCM, washed saturated NaHCO₃ (aq.), dried with Na₂SO₄ and evaporated. The residue purified by column chromatography on silica gel. Yield of compound P67: 1.0 g, 68%. ¹H NMR (400 MHz, DMSO-d₆), δ: 8.03-7.88 (m, 2H), 7.66 (t, J=7.4 Hz, 1H), 7.53 (t, J=7.7 Hz, 2H), 4.30 (t, J=6.5 Hz, 2H), 3.95 (s, 2H), 3.49 (t, J=6.3 Hz, 2H), 1.86-1.71 (m, 2H), 1.71-1.54 (m, 2H), 1.41 (s, 9H).

### Preparation 68. [4-(Benzoyloxy)butoxy]acetic acid (P68)

To a solution of Compound P67 (1.0 g, 3.3 mmol) in 15 mL of DCM, TFA (2.6 mL, 33.0 mmol) was added. The mixture was stirred at rt for 14 h. The mixture was evaporated. Yield of compound P68: 0.84 g, 99%. ¹H NMR (400 MHz, DMSO-d₆), δ: 12.56 (s, 1H), 7.97 (d, J=7.2 Hz, 2H), 7.66 (t, J=7.4 Hz, 1H), 7.53 (t, J=7.7 Hz, 2H), 4.30 (t, J=6.5 Hz, 2H), 3.98 (s, 2H), 3.50 (t, J=6.3 Hz, 2H), 1.87-1.71 (m, 2H), 1.65 (dt, J=13.2, 6.5 Hz, 2H).

### Synthesis of 4-{[3-(5-Amino-3-methylpyridin-2-yl)-1,2,4-oxadiazol-5-yl]methoxy}butyl benzoate (P70)

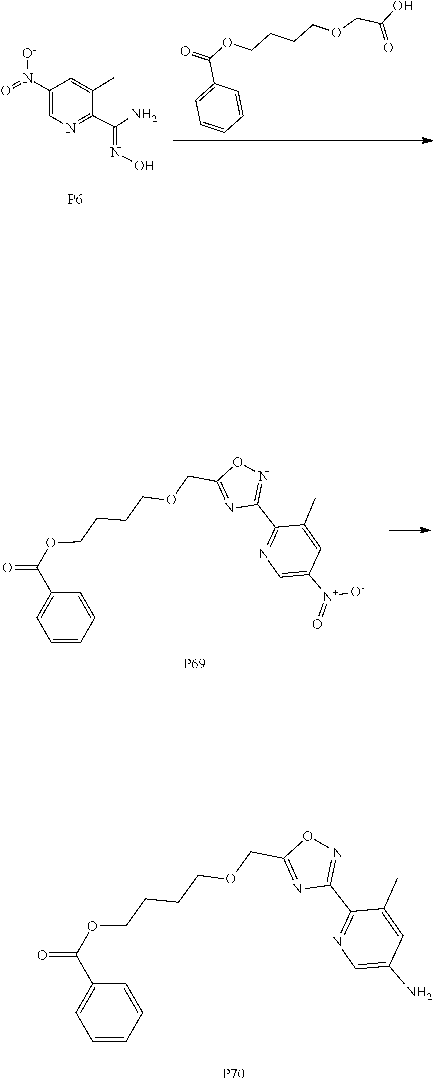

### Preparation 69. 4-{[3-(3-Methyl-5-nitropyridin-2-yl)-1,2,4-oxadiazol-5-yl]methoxy}butyl benzoate (P69)

To a solution of Compound P68 (0.84 g, 3.3 mmol) in 20 mL of dioxane, CDI (0.6 g, 3.7 mmol) was added. The mixture was stirred at 60° C. for 4 h. Then Compound P6 (0.65 g, 3.3 mmol) was added, and the mixture was stirred for 72 h at reflux. The mixture was evaporated. The residue was diluted with water and extracted with ethyl acetate. Combined organic extracts were dried with Na₂SO₄ and evaporated. The residue was purified by column chromatography on silica gel. Yield of compound P69: 0.25 g, 18%. LCMS ESI (m/z): 413.5 ([M+1]⁺), 291.5.

### Preparation 70. 4-{[3-(5-Amino-3-methylpyridin-2-yl)-1,2,4-oxadiazol-5-yl]methoxy}butyl benzoate (P70)

To a solution of compound P69 (0.25 g, 0.6 mmol) in 10 mL of acetic acid, Fe powder (0.3 g, 6.0 mmol) was added. The suspension was stirred at 70° C. for 1 h. The reaction mixture was cooled to ambient temperature, diluted with EtOAc (100 mL) and filtered through Celite. The filtrate was evaporated. The residue was diluted with saturated NaHCO₃ (aq.) and extracted with ethyl acetate. Combined organic extracts were dried with Na₂SO₄ and evaporated. The residue was purified by column chromatography on silica gel. Yield of compound P70: 0.14 g, 60%. LCMS ESI (m/z): 383.5 ([M+1]⁺).

### Synthesis of N-(7-cyclopentylpyrazolo[1,5-a]pyrimidin-6-yl)-N-(5-methyl-6-{5-[(4-oxobutoxy)methyl]-1,2,4-oxadiazol-3-yl}pyridin-3-yl)urea (P73)

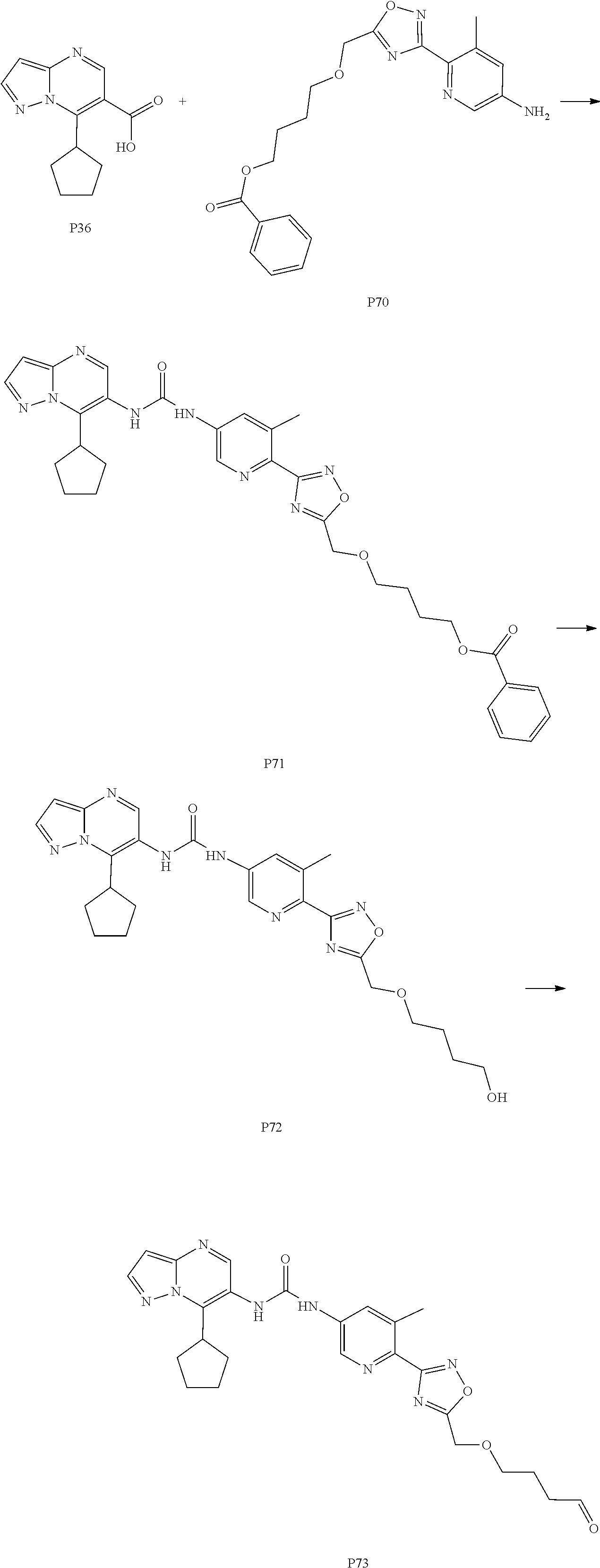

### Preparation 71. 4-({3-[5-({[(7-Cyclopentylpyrazolo[1,5-a]pyrimidin-6-yl)amino]carbonyl}amino)-3-methylpyridin-2-yl]-1,2,4-oxadiazol-5-yl}methoxy)butyl benzoate (P71)

To a solution of Compound P36 (0.08 g, 0.035 mmol) in 10 mL of toluene, Et₃N (0.2 mL, 0.140 mmol) was added. The mixture was stirred at rt for 10 min. Then, DPPA (0.09 mL, 0.042 mmol) was added. The mixture was stirred at rt for additional 1 h, and Compound P70 (0.13 g, 0.035 mmol) was added. The resulting mixture was stirred for 14 h at 90° C. The mixture was evaporated and purified by column chromatography on silica gel. Yield of compound P71: 0.13 g, 58%. LCMS ESI (m/z): 611.5 ([M+1]⁺).

### Preparation 72. N-(7-cyclopentylpyrazolo[1,5-a]pyrimidin-6-yl)-N-(6-{5-[(4-hydroxybutoxy)methyl]-1,2,4-oxadiazol-3-yl}-5-methylpyridin-3-yl)urea (P72)

To a solution of Compound P71 (0.13 g, 0.21 mmol) in 5 mL of MeOH, K₂CO₃ (0.12 g, 0.84 mmol) was added. The mixture was stirred at 60° C. for 4 h. The mixture was evaporated. The residue was diluted with water and extracted with ethyl acetate. Combined organic extracts were dried with Na₂SO₄ and evaporated. Yield of compound P72: 0.085 g, 80%. LCMS ESI (m/z): 507.5 ([M+1]⁺).

### Preparation 73. N-(7-cyclopentylpyrazolo[1,5-a]pyrimidin-6-yl)-N′-(5-methyl-6-{5-[(4-oxobutoxy)methyl]-1,2,4-oxadiazol-3-yl}pyridin-3-yl)urea (P73)

A solution of dimethyl sulfoxide (0.04 mL, 0.5 mmol) in DCM (0.1 mL) was added to a stirred solution of oxalyl chloride (0.032 mL, 0.36 mmol) in DCM (10 mL) at −70° C. A solution of Compound P72 (0.078 g, 0.15 mmol) in DCM (1.0 mL) was added dropwise to the above prepared solution over a period of 15 min and the mixture was stirred for an additional 3.5 h. Triethylamine (0.13 mL, 0.90 mmol) was added dropwise with stirring for 15 min, and the whole was allowed to warm to rt. The mixture was diluted with DCM, washed saturated NaHCO₃ (aq.), dried with Na₂SO₄ and evaporated. The residue (P73) was used on the next step without additional purification. LCMS ESI (m/z): 505.4 ([M+1]⁺).

### Synthesis of 3-[2-(Benzoyloxy)ethoxy]propanoic acid (P75)

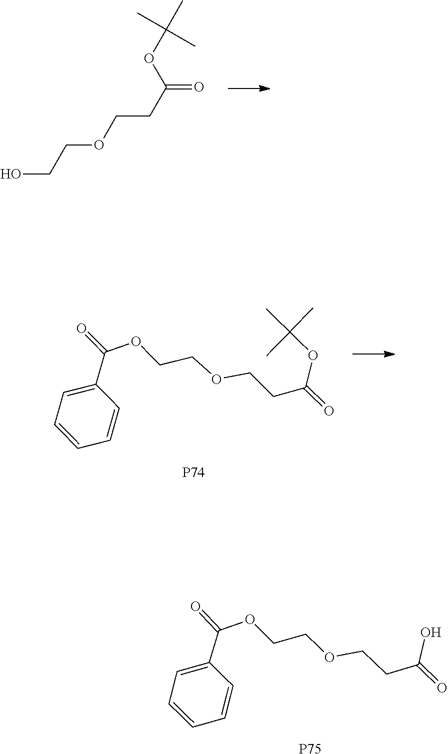 [SMILES: CC(C)(C)OC(=O)CCOCCO ; "P74" ; CC(C)(C)OC(=O)CCOCCOC(=O)c1ccccc1 ; "P75" ; O=C(O)CCOCCOC(=O)c1ccccc1]

### Preparation 74. 2-(3-Tert-butoxy-3-oxopropoxy)ethyl benzoate (P74)

To a solution of tert-butyl 3-(2-hydroxyethoxy)propanoate (1.0 g, 5.3 mmol) in 20 mL of DCM, Et₃N (1.9 mL, 13.3 mmol) was added. After this, benzoyl chloride (0.7 mL, 5.9 mmol) was added at 0-5° C. The mixture was stirred at rt for 14 h. The mixture was diluted with DCM, washed with saturated NaHCO₃ (aq.), dried with Na₂SO₄, and evaporated. The residue was purified by column chromatography on silica gel. Yield of compound P74: 1.2 g, 80%. ¹H NMR (400 MHz, DMSO-d₆), δ: 7.96 (d, J=7.8 Hz, 2H), 7.66 (t, J=7.4 Hz, 1H), 7.53 (t, J=7.6 Hz, 2H), 4.38 (d, J=4.5 Hz, 2H), 3.73 (d, J=4.5 Hz, 2H), 3.66 (t, J=6.1 Hz, 2H), 2.43 (t, J=6.1 Hz, 2H), 1.37 (d, J=13.7 Hz, 9H).

### Preparation 75. 3-[2-(Benzoyloxy)ethoxy]propanoic acid (P75)

To a solution of Compound P74 (1.2 g, 4.1 mmol) in 15 mL of DCM, TFA (3.2 mL, 41.0 mmol) was added. The mixture was stirred at rt for 14 h. The mixture was evaporated. Yield of compound P75: 0.96 g, 99%. ¹H NMR (400 MHz, DMSO-d₆), δ: 12.87-11.46 (m, 1H), 8.08-7.91 (m, 2H), 7.66 (t, J=7.4 Hz, 1H), 7.53 (t, J=7.7 Hz, 2H), 4.47-4.29 (m, 2H), 3.76-3.71 (m, 2H), 3.68 (t, J=6.3 Hz, 2H), 2.50 (d, J=1.7 Hz, 4H), 2.46 (t, J=6.3 Hz, 2H).

### Synthesis of 4-(2,7-Diazaspiro[3.5]non-2-yl)-6-(2-phenylethyl)quinazoline (P78)

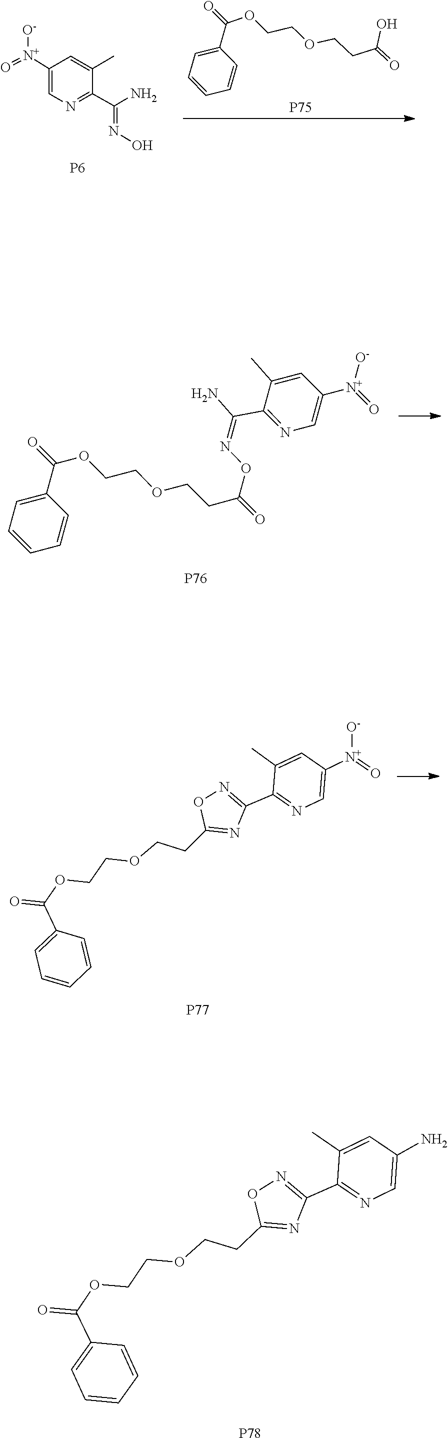

### Preparation 76. 2-[3-({[(1E)-Amino(3-methyl-5-nitropyridin-2-yl)methylene]amino}oxy)-3-oxopropoxy]ethyl benzoate (P76)

To a solution of Compound P75 (0.48 g, 2.0 mmol) in 20 mL of DCM, Et₃N (0.9 mL, 6.0 mmol) was added. The mixture was stirred at rt for 10 min. Then, TBTU (0.6 g, 2.4 mmol) and Compound P6 (0.40 g, 2.0 mmol) were added, and the mixture was stirred for 14 h at ambient temperature. The mixture was washed with 20% K₂CO₃ (aq.), dried with Na₂SO₄ and evaporated. The residue (P76) was used on the next step without additional purification. LCMS ESI (m/z): 417.6 ([M+1]⁺), 179.3, 149.4.

### Preparation 77. 2-{2-[3-(3-Methyl-5-nitropyridin-2-yl)-1,2,4-oxadiazol-5-yl]ethoxy}ethyl benzoate (P77)

A solution of compound P76 in 50 mL of dioxane was stirred at reflux for 96 h. The mixture was evaporated and purified by column chromatography on silica gel. Yield of compound P77: 0.5 g, 62% on 2 steps (Preparation 76 and Preparation 77). ¹H NMR (400 MHz, DMSO-d₆), δ: 9.31 (d, J=2.4 Hz, 1H), 8.69 (d, J=1.9 Hz, 1H), 7.92-7.82 (m, 2H), 7.58 (t, J=7.4 Hz, 1H), 7.43 (t, J=7.8 Hz, 2H), 4.40-4.34 (m, 2H), 3.98 (t, J=6.1 Hz, 2H), 3.83-3.75 (m, 2H), 3.35 (t, J=6.0 Hz, 2H), 2.58 (s, 3H).

### Preparation 78. 2-{2-[3-(5-Amino-3-methylpyridin-2-yl)-1,2,4-oxadiazol-5-yl]ethoxy}ethyl benzoate (P78)

To a solution of compound P77 (0.5 g, 1.3 mmol) in 10 mL of acetic acid, Fe powder (0.7 g, 13.0 mmol) was added. The suspension was stirred at 70° C. for 1 h. The reaction mixture was cooled to ambient temperature, diluted with EtOAc (100 mL), and filtered through Celite. The filtrate was evaporated. The residue was diluted with saturated NaHCO₃ (aq.) and extracted with ethyl acetate. Combined organic extracts were dried with Na₂SO₄ and evaporated. The residue was purified by column chromatography on silica gel. Yield of compound P78: 0.42 g, 91%. LCMS ESI (m/z): 369.1 ([M+1]⁺), 149.4.

### Synthesis of N-(7-cyclopentylpyrazolo[1,5-a]pyrimidin-6-yl)-N-(5-methyl-6-{5-[2-(2-oxoethoxy)ethyl]-1,2,4-oxadiazol-3-yl}pyridin-3-yl)urea (P81)

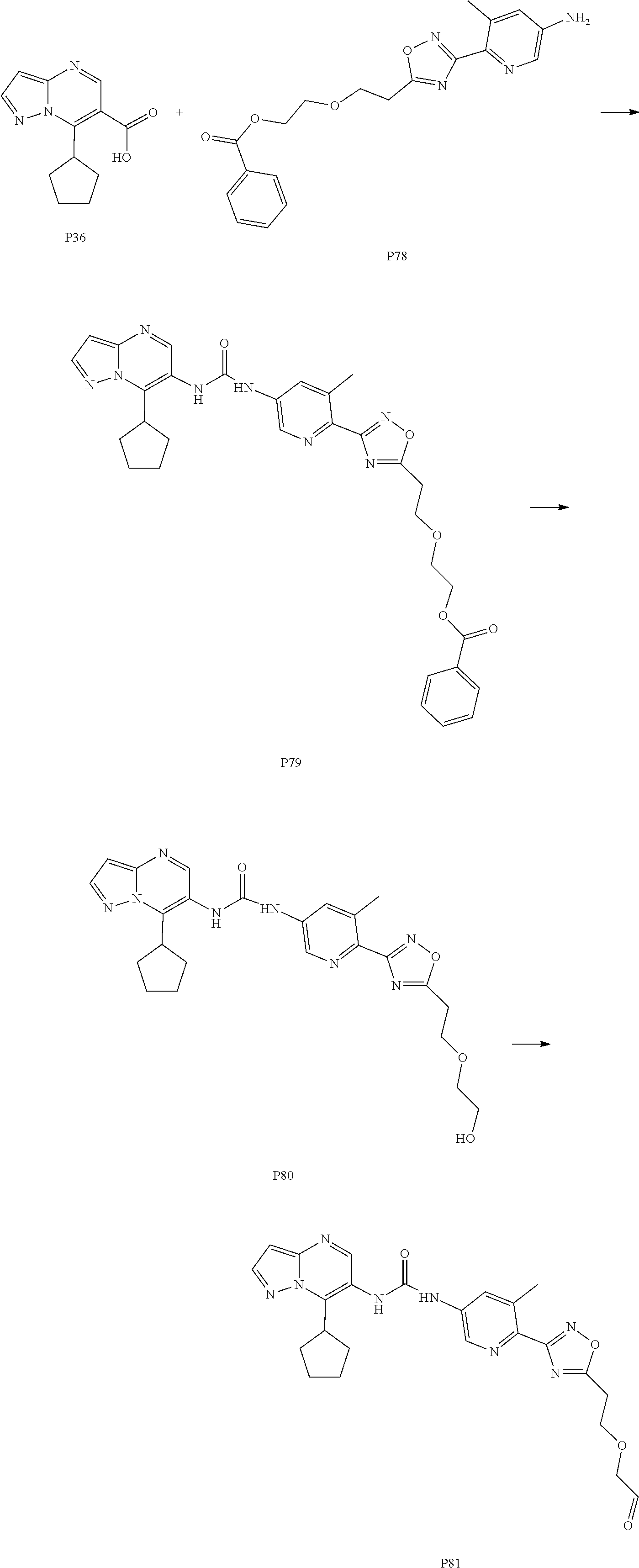

### Preparation 79. 2-(2-{3-[5-({[(7-Cyclopentylpyrazolo[1,5-a]pyrimidin-6-yl)amino]carbonyl}amino)-3-methylpyridin-2-yl]-1,2,4-oxadiazol-5-yl}ethoxy)ethyl benzoate (P79)

To a solution of Compound P36 (0.29 g, 1.3 mmol) in 20 mL of toluene, Et₃N (0.7 mL, 5.2 mmol) was added. The mixture was stirred at rt for 10 min. Then, DPPA (0.32 mL, 1.6 mmol) was added. The mixture was stirred at rt for 1 h, and Compound P78 (0.42 g, 1.3 mmol) was added. The resulting mixture was stirred at 90° C. for 14 h. The mixture was evaporated and purified by column chromatography on silica gel. Yield of compound P79: 0.5 g, 66%. LCMS ESI (m/z): 597.3 ([M+1]⁺).

### Preparation 80. N-(7-cyclopentylpyrazolo[1,5-a]pyrimidin-6-yl)-N′-(6-{5-[2-(2-hydroxyethoxy)ethyl]-1,2,4-oxadiazol-3-yl}-5-methylpyridin-3-yl)urea (P80)

To a solution of Compound P79 (0.23 g, 0.4 mmol) in 5 mL of MeOH, K₂CO₃ (0.16 g, 1.2 mmol) was added. The mixture was stirred at ambient temperature for 4 h. The mixture was evaporated. The residue was diluted with water and extracted with ethyl acetate. Combined organic extracts were dried with Na₂SO₄ and evaporated. Yield of compound P80: 0.1 g, 54%. LCMS ESI (m/z): 493.6 ([M+1]⁺).

### Preparation 81. N-(7-cyclopentylpyrazolo[1,5-a]pyrimidin-6-yl)-N-(5-methyl-6-{5-[2-(2-oxoethoxy)ethyl]-1,2,4-oxadiazol-3-yl}pyridin-3-yl)urea (P82)

A solution of DMSO (0.08 mL, 1.2 mmol) in DCM (0.1 mL) was added to a stirred solution of oxalyl chloride (0.08 mL, 0.86 mmol) in DCM (10 mL) at −70° C. A solution of Compound P81 (0.18 g, 0.36 mmol) in DCM (1.0 mL) was added dropwise to the above prepared solution over a period of 15 min and the mixture was stirred for an additional 3.5 h. Triethylamine (0.3 mL, 2.16 mmol) was added dropwise with stirring for 15 min, and the whole was allowed to warm to rt. The mixture was diluted with DCM, washed with saturated NaHCO₃ (aq.), dried with Na₂SO₄, and evaporated. The residue (P82) was used on the next step without additional purification. LCMS ESI (m/z): 491.4 ([M+1]⁺).

### Synthesis of 6-{3-[5-({[(5-Cyclopentylimidazo[1,2-a]pyrimidin-6-yl)amino]carbonyl}amino)-3-methylpyridin-2-yl]-1,2,4-oxadiazol-5-yl}hexanoic acid (P82)

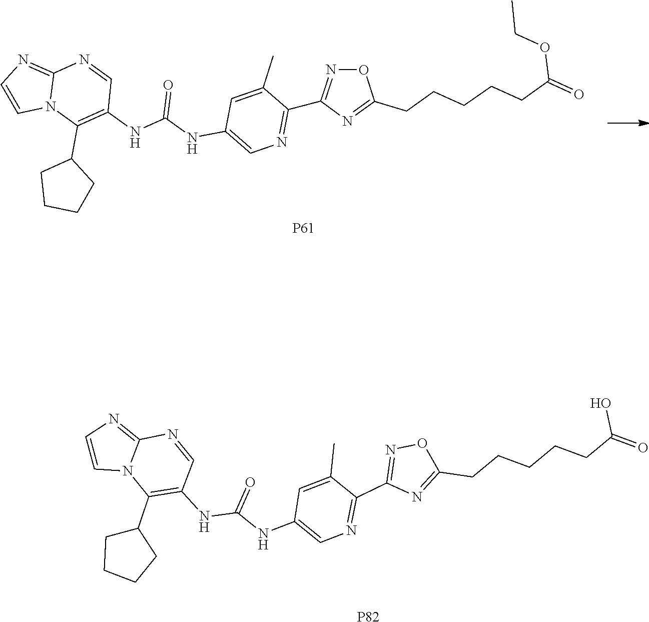

### Preparation 82. 6-{3-[5-({[(5-Cyclopentylimidazo[1,2-a]pyrimidin-6-yl)amino]carbonyl}amino)-3-methylpyridin-2-yl]-1,2,4-oxadiazol-5-yl}hexanoic acid (P82)

Aqueous solution of NaOH (0.015 g, 0.37 mmol) was added into a solution of Compound P61 (0.101 g, 0.23 mmol) in 10 ml of ethanol. The mixture was stirred for 12 h at 50° C. After the full hydrolysis of the ester, the mixture was cooled down to ambient temperature. Ethanol was evaporated and the reaction mixture was acidified with hydrochloric acid to pH=7, the product was then extracted with ethyl acetate. Solution of the product in ethyl acetate was dried with Na₂SO₄ and evaporated. Compound P82 (0.086 g, 90%) was obtained as a solid. LCMS ESI (m/z): 519.3 ([M+1]⁺).

### Synthesis of 5-Methoxy-3,3-dimethyl-5-oxopentanoic acid (P84)

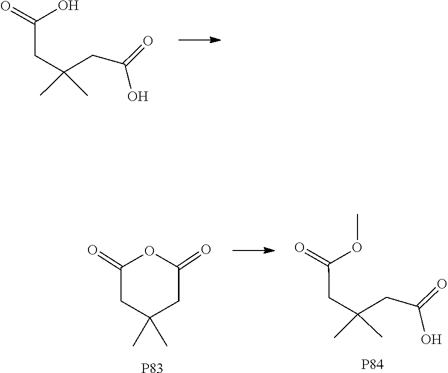

### Preparation 83. 4,4-Dimethyldihydro-2H-pyran-2,6(3H)-dione (P83)

To 3,3-Dimethylpentanedioic acid (1.0 g, 6.20 mmol) was added acetyl chloride (1.96 g, 24.8 mmol). This mixture was then heated at 60° C. for 3 h and was observed to go from a suspension to a homogeneous solution. The volatiles were then removed at 80° C. under reduced pressure leaving behind a solid, giving Compound P83. This was used immediately in the next reaction without further purification.

### Preparation 84. tert-Butyl 2-[6-(phenylethynyl)quinazolin-4-yl]-2,7-diazaspiro[3.5]nonane-7-carboxylate (P84)

The anhydride (Compound P83) from the previous stage was dissolved in dry MeOH (50 ml), metallic sodium (0.144 g, 6.2 mmol) was added, and the reaction mixture was stirred at reflux for 12 h. The mixture was then acidified to pH 4 with AcOH, and the methanol was removed under reduced pressure. The residue was dissolved in water (100 ml) and extracted with EtOAc. The organic layer was washed with brine, dried (Na₂SO₄), and concentrated in vacuo to yield a colorless, viscous oil of Compound P84 (0.537 g, 49%). ¹H NMR (400 MHz, DMSO-d₆), δ: 3.57 (s, 3H), 2.39 (s, 2H), 2.27 (s, 2H), 1.04 (s, 6H).

### Synthesis of 4-{3-[5-({[(7-Cyclopentylpyrazolo[1,5-a]pyrimidin-6-yl)amino]carbonyl}amino)-3-methylpyridin-2-yl]-1,2,4-oxadiazol-5-yl}-3,3-dimethylbutanoic acid (P88)

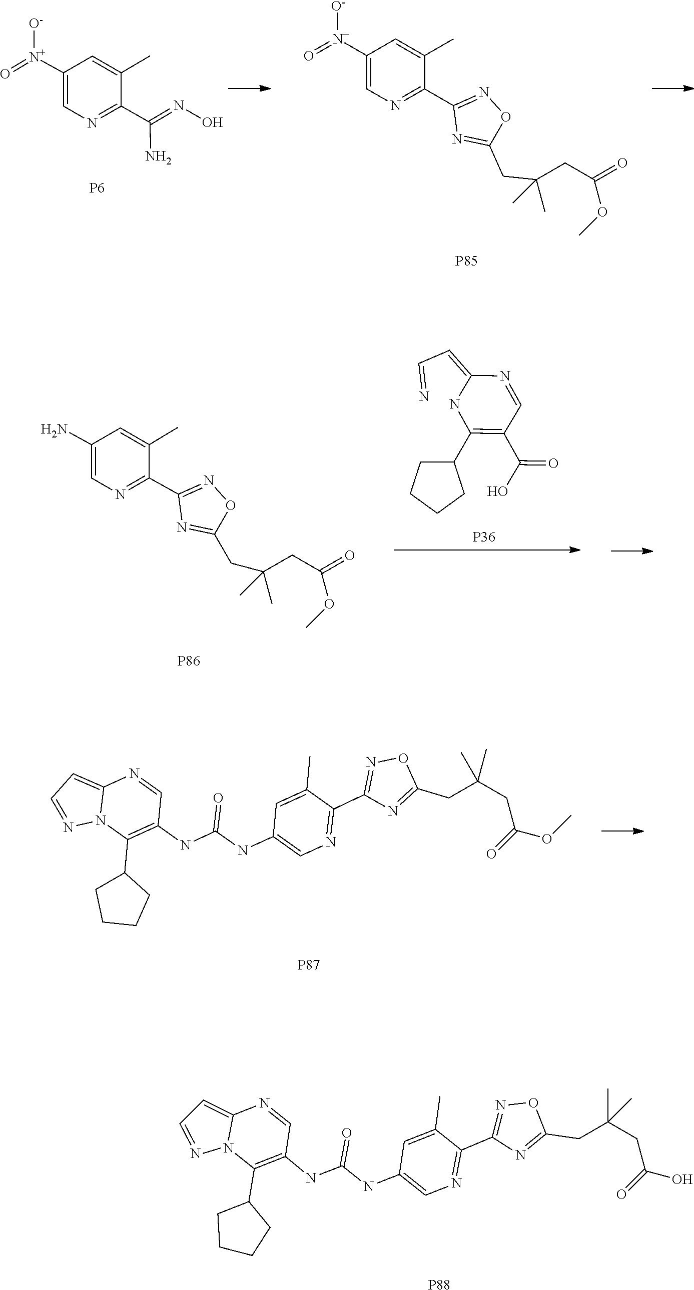

### Preparation 85. Methyl 3,3-dimethyl-4-[3-(3-methyl-5-nitropyridin-2-yl)-1,2,4-oxadiazol-5-yl]butanoate (P85)

To a solution of Compound P84 (0.444 g, 2.5 mmol) in 20 ml of 1,4-dioxane, 1,1′-carbonylbis-1H-imidazole (0.645 g, 4.98 mmol) was added. The solution was stirred at 50° C. for 3 h, then Compound P6 (0.5 g, 2.5 mmol) was added, and the mixture was refluxed for 200 h. After completion of the reaction as indicated by TLC, the reaction mixture was concentrated under vacuum, dissolved in ethyl acetate, and washed with water and evaporated. The residue was purified on silica gel eluting with EtOAc/hexane, 1:5, giving Compound P85 (0.1 g, 11%). ¹H NMR (400 MHz, CDCl₃), δ: 9.44 (s, 1H), 8.49 (s, 1H), 3.71 (s, 3H), 3.21 (s, 2H), 2.81 (s, 3H), 2.47 (s, 2H), 1.21 (s, 6H).

### Preparation 86. Methyl 4-[3-(5-amino-3-methylpyridin-2-yl)-1,2,4-oxadiazol-5-yl]-3,3-dimethylbutanoate (P86)

Compound P85 (0.1 g, 0.3 mmol) was dissolved in 30 ml of a solvent consisting of ethanol and acetic acid in equal amounts. The mixture was heated to 40° C. and five equivalents of Fe powder (0.0835 g, 1.5 mmol) were added. The stirring was continued at this temperature until the starting substance was completely converted as indicated by TLC. The mixture was cooled down to ambient temperature and passed through Celite. The filtrate was evaporated, and the residue was dissolved in ethyl acetate and washed with water and concentrated. Then the mixture was dissolved in DCM, washed with an aqueous solution of sodium bicarbonate, dried over sodium sulfate and concentrated, giving Compound P86 (0.06 g, 66%). LCMS ESI (m/z): 305.5 ([M+1]⁺).

### Preparation 87. Methyl 4-{3-[5-({[(7-cyclopentylpyrazolo[1,5-a]pyrimidin-6-yl)amino]carbonyl}amino)-3-methylpyridin-2-yl]-1,2,4-oxadiazol-5-yl}-3,3-dimethylbutanoate (P87)

Triethylamine (80.6 mg, 0.8 mmol) was added to a suspension of 7-cyclopentylpyrazolo[1,5-a]pyrimidine-6-carboxylic acid P36 (46.0 mg, 0. 2 mmol) in 10 ml of toluene. The mixture was stirred for 5 min. Then DPPA (65.7 mg, 0.24 mmol) was added and stirred for 60 min at rt. Then Compound P86 (60.5 mg, 0.2 mmol) was added, and the mixture was refluxed for 12 h. Reaction was monitored by LCMS. The mixture was quenched with water, separated toluene was dried with Na₂SO₄ and evaporated. The residue was purified on silica gel eluting with hexane/ethyl acetate, 1:1, giving 4-{3-[5-({[(7-cyclopentylpyrazolo[1,5-a]pyrimidin-6-yl)amino]carbonyl}amino)-3-methylpyridin-2-yl]-1,2,4-oxadiazol-5-yl}-3,3-dimethylbutanoate P87 (41 mg, 39%). LCMS (m/z): 533.3 ([M+1]⁺).

### Preparation 88. 4-{3-[5-({[(7-Cyclopentylpyrazolo[1,5-a]pyrimidin-6-yl)amino]carbonyl}amino)-3-methylpyridin-2-yl]-1,2,4-oxadiazol-5-yl}-3,3-dimethylbutanoic acid (P88)

An aqueous solution of NaOH (6.2 mg, 0.154 mmol) was added into a solution of Compound P87 (41 mg, 0.23 mmol) in 10 ml of ethanol. The mixture was stirred for 12 h at 50° C. After the full hydrolysis of the ester, the mixture was cooled down to ambient temperature. Ethanol was evaporated and the reaction mixture was acidified with hydrochloric acid to pH=7, the product was then extracted with ethyl acetate. Solution of the product in ethyl acetate was dried with Na₂SO₄ and evaporated. Compound P88 (35.9 g, 90%) was obtained as a solid. ¹H NMR (400 MHz, DMSO-d₆), δ: 11.99 (s, 1H), 9.36 (s, 1H), 8.62 (d, J=2.2 Hz, 1H), 8.51 (s, 1H), 8.48 (s, 1H), 8.23 (d, J=2.3 Hz, 1H), 7.99 (s, 1H), 6.75 (d, J=2.3 Hz, 1H), 3.95-3.74 (m, 1H), 3.00 (t, J=7.4 Hz, 2H), 2.47 (s, 3H), 2.34 (d, J=8.5 Hz, 2H), 2.22 (t, J=7.3 Hz, 2H), 1.89 (d, J=9.4 Hz, 2H), 1.78 (dd, J=14.9, 7.2 Hz, 2H), 1.71 (s, 2H), 1.54 (dd, J=15.1, 7.4 Hz, 2H), 1.39 (dd, J=14.8, 8.0 Hz, 2H).

### Synthesis of Ethyl 5-[3-(3-methyl-5-nitropyridin-2-yl)-1,2,4-oxadiazol-5-yl]pentanoate (P48)

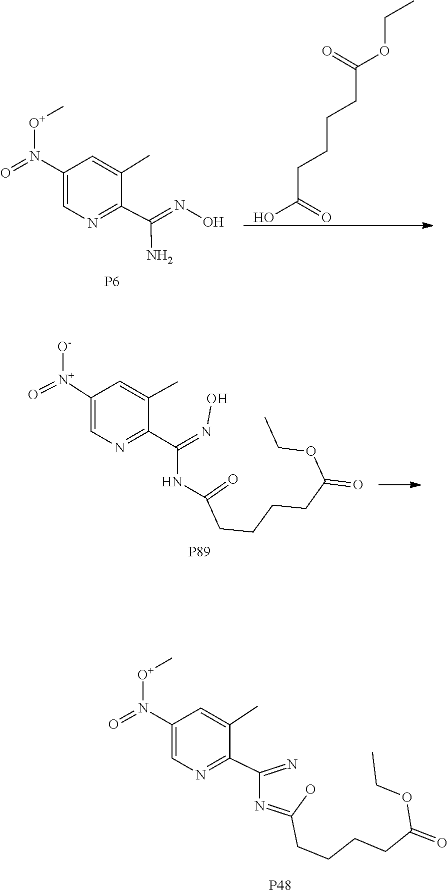

### Preparation 89. Ethyl 6-{[(E)-(hydroxyimino)(3-methyl-5-nitropyridin-2-yl)methyl]amino}-6-oxohexanoate (P89)

To a solution of 6-ethoxy-6-oxohexanoic acid (0.355 g, 2.04 mmol) in 40 ml of DCM, TEA (0.825 g, 8.16 mmol) was added. The solution was stirred for 5 min, then O-(Benzotriazol-1-yl)-N,N,N′,N′-tetramethyluronium tetrafluoroborate (0.786 g, 2.45 mmol) was added, and the mixture was stirred for 10 min. Compound P6 (0.4 g, 2.04 mmol) was added into the reaction flask and the mixture was stirred for 12 h at rt. After completion of the reaction as indicated by TLC, the reaction mixture was concentrated under vacuum, dissolved in ethyl acetate, and washed with water and evaporated. The residue was used for the next step without additional purification. LCMS ESI (m/z): 353.5 ([M+1]⁺).

### Preparation 48a. Ethyl 5-[3-(3-methyl-5-nitropyridin-2-yl)-1,2,4-oxadiazol-5-yl]pentanoate (P48)

Compound P89 (0.718 g, 2.04 mmol) was dissolved in 40 ml of 1,4-dioxane. The solution was refluxed for 192 h. After completion of the reaction as indicated by LCMS, the reaction mixture was concentrated under vacuum. The residue was purified on silica gel eluting with EtOAc/hexane, 1:10, to yield the Compound P48 (0.134 g). The yield was 20% for two steps. LCMS ESI (m/z): 335.5 ([M+1]⁺).

### Synthesis of N-{5-Chloro-6-[5-(4-oxobutyl)-1,2,4-oxadiazol-3-yl]pyridin-3-yl}-N-(7-cyclopentylpyrazolo[1,5-a]pyrimidin-6-yl)urea (P96)

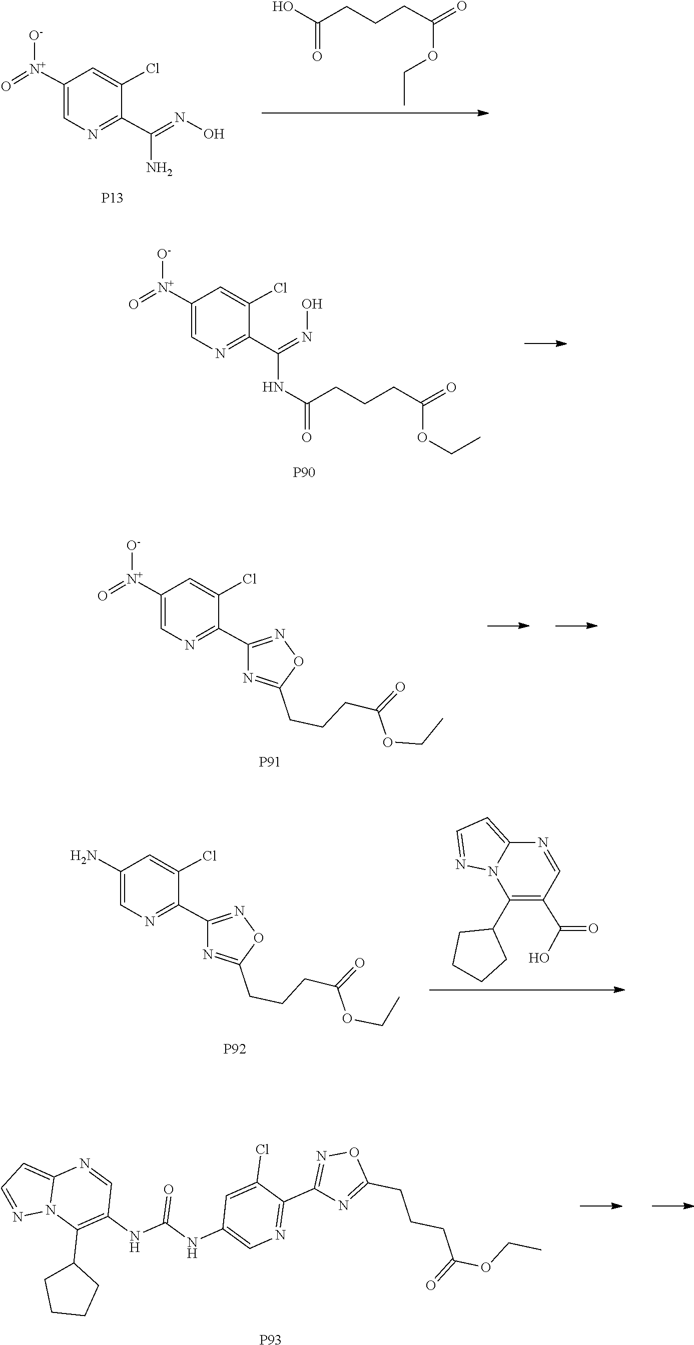

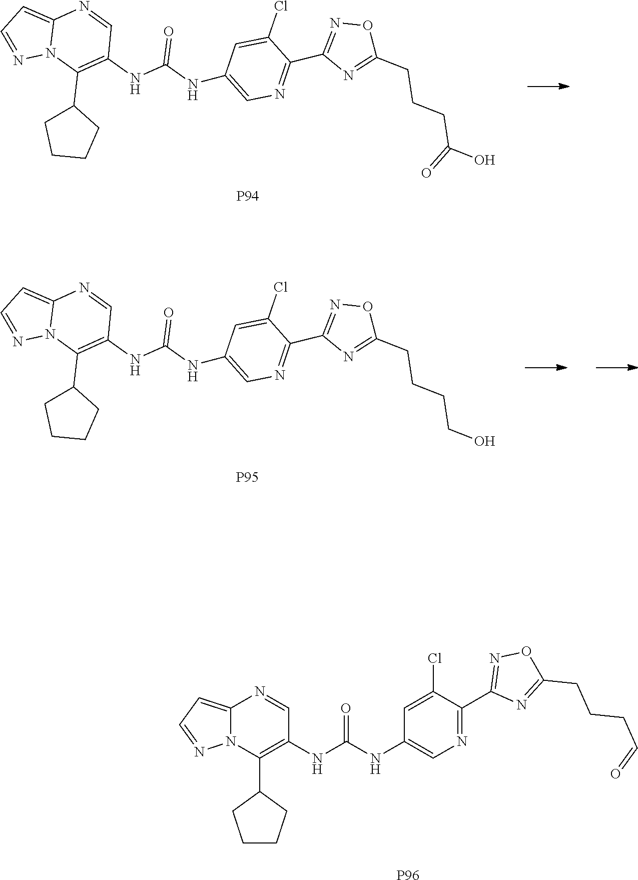

### Preparation 90. Ethyl 5-{[(E)-(3-chloro-5-nitropyridin-2-yl)(hydroxyimino)methyl]amino}-5-oxopentanoate (P90)

To a solution of 5-ethoxy-5-oxopentanoic acid (0.74 g, 4.62 mmol) in 50 ml of DCM, TEA (1.87 g, 18.48 mmol) was added. The solution was stirred for 5 min, then O-(Benzotriazol-1-yl)-N,N,N′,N′-tetramethyluronium tetrafluoroborate (1.78 g, 5.54 mmol) was added, and the mixture was stirred for 10 min. Compound P13 (1.0 g, 4.62 mmol) was added into the reaction flask and the mixture was stirred for 12 h at rt. After completion of the reaction as indicated by TLC, the reaction mixture was concentrated under vacuum, dissolved in ethyl acetate, and washed with water and evaporated. The residue was used for the next step without additional purification.

### Preparation 91. Ethyl 4-[3-(3-chloro-5-nitropyridin-2-yl)-1,2,4-oxadiazol-5-yl]butanoate (P91)

Compound P90 (1.66 g, 4.62 mmol) was dissolved in 50 ml of 1,4-dioxane. The solution was refluxed for 200 h. After completion of the reaction as indicated by LCMS, the reaction mixture was concentrated under vacuum. The residue was purified on silica gel eluting with EtOAc/hexane, 1:10, giving the Compound P91 (0.398 g). The yield was 25% for two steps. LCMS ESI (m/z): 341.1 ([M+1]⁺), 295.3.

### Preparation 92. Ethyl 4-[3-(5-amino-3-chloropyridin-2-yl)-1,2,4-oxadiazol-5-yl]butanoate (P92)

Compound P91 (0.398 g, 1.17 mmol) was dissolved in 20 ml of a solvent consisting of ethanol and acetic acid in equal amounts. The mixture was heated to 40° C. and five equivalents of Fe powder (0.326 g, 5.85 mmol) were added. The stirring was continued at this temperature until the starting substance was completely converted as indicated by TLC. The mixture was cooled down to ambient temperature and passed through Celite. The filtrate was evaporated, and the residue was dissolved in ethyl acetate and washed with water and concentrated. Then the mixture was dissolved in DCM, washed with an aqueous solution of NaHCO₃, dried over Na₂SO₄ and concentrated, giving the Compound P92 (0.254 g, 70%). LCMS ESI (m/z): 311.4 ([M+1]⁺), 265.0.

### Preparation 93. Ethyl 4-{3-[3-chloro-5-({[(7-cyclopentylpyrazolo[1,5-a]pyrimidin-6-yl)amino]carbonyl}amino)pyridin-2-yl]-1,2,4-oxadiazol-5-yl}butanoate (P93)

Triethylamine (0.331 g, 3.27 mmol) was added to a suspension of 7-cyclopentylpyrazolo[1,5-a]pyrimidine-6-carboxylic acid (0.189 g, 0.817 mmol) in 20 ml of toluene. The mixture was stirred for 5 min. Then DPPA (0.27 g, 0.98 mmol) was added and stirred for 60 min at rt. After this Compound P92 (0.254 g, 0.817 mmol) was added and the mixture was refluxed for 12 h. Reaction was monitored by LCMS. The mixture was quenched with water, separated toluene was dried with Na₂SO₄ and evaporated. The residue was purified on silica gel eluting with hexane/ethyl acetate, 1:1, giving the Compound P93 (0.284 g, 64%). LCMS ESI (m/z): 539.3 ([M+1]⁺).

### Preparation 94. 4-{3-[3-Chloro-5-({[(7-cyclopentylpyrazolo[1,5-a]pyrimidin-6-yl)amino]carbonyl}amino)pyridin-2-yl]-1,2,4-oxadiazol-5-yl}butanoic acid (P94)

Aqueous solution of LiOH (0.0252 g, 1.054 mmol) was added into a solution of Compound P93 (0.284 g, 0.527 mmol) in 20 ml of THF. The mixture was stirred for 12 h at 50° C. After the full hydrolysis of the ester, the mixture was cooled down to ambient temperature. THF was evaporated and the reaction mixture was acidified with hydrochloric acid to pH=7, the product was then extracted with ethyl acetate. Solution of the product in ethyl acetate was dried with Na₂SO₄ and evaporated. Compound P94 (0.269 g, 99%) was obtained as a solid. LCMS ESI (m/z): 511.6 ([M+1]⁺), 379.5.

### Preparation 95. N-{5-chloro-6-[5-(4-hydroxybutyl)-1,2,4-oxadiazol-3-yl]pyridin-3-yl}-N-(7-cyclopentylpyrazolo[1,5-a]pyrimidin-6-yl)urea (P95)

To a solution of Compound P94 (0.047 g, 0.09 mmol) in 10 mL of THF, Et₃N (0.012 g, 1.12 mmol) was added. The mixture was stirred at rt for 10 min. Then, ethyl chloroformate (0.013 g, 1.12 mmol) was added at 0-5° C. The mixture was stirred at rt for 1 h. The formed solid was filtered off and the filtrate was added dropwise to a solution of sodium borohydride (0.017 g, 0.45 mmol) in water (10 mL) at 0-5° C. The resulting mixture was stirred for 24 h at ambient temperature. The mixture was diluted with water and extracted with ethyl acetate. Combined organic extracts were dried with Na₂SO₄ and evaporated. The residue purified by column chromatography on silica gel. Yield of compound P95: 0.35 g, 76%. LCMS ESI (m/z): 497.4 ([M+1]⁺).

### Preparation 96. N-{5-Chloro-6-[5-(4-oxobutyl)-1,2,4-oxadiazol-3-yl]pyridin-3-yl}-N′-(7-cyclopentylpyrazolo[1,5-a]pyrimidin-6-yl)urea (P96)

A solution of dimethyl sulfoxide (12.4 mg, 0.158 mmol) in DCM (0.1 mL) was added to a stirred solution of oxalyl chloride (14.6 mg, 0.115 mmol) in DCM (10 mL) at −70° C. A solution of Compound P95 (24 mg, 0.048 mmol) in DCM (1.0 mL) was added dropwise to the above prepared solution over a period of 15 min and the mixture was stirred for an additional 3.5 h. Triethylamine (29 mg, 0.288 mmol) was added dropwise with stirring for 15 min, and the whole was allowed to warm to rt. The mixture was diluted with DCM, washed saturated NaHCO₃ (aq.), dried with Na₂SO₄ and evaporated. The residue P96 was used on the next step without additional purification. LCMS ESI (m/z): 495.6 ([M+1]⁺).

### Synthesis of 7-(3,4,5-Trimethoxyphenyl)pyrazolo[1,5-a]pyrimidine-6-carboxylic acid (P100)

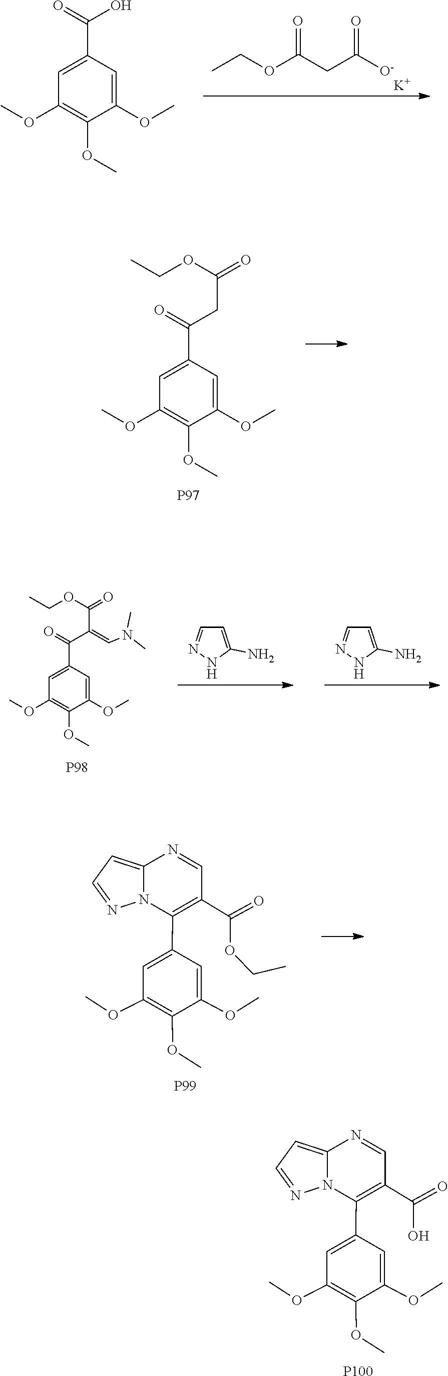

### Preparation 97. Ethyl 3-oxo-3-(3,4,5-trimethoxyphenyl)propanoate (P97)

1) Carbonyldiimidazole (4.2 g, 26.0 mmol) was added in portions to a mixture of 3,4,5-trimethoxybenzoic acid (5.0 g, 23.60 mmol) and anhydrous EtOAc (100 mL). The mixture was stirred for 3 h at 50° C.

2) MgCl₂ (9.0 g, 94.4 mmol) was added in portions to a mixture of potassium 3-ethoxy-3-oxo-propanoate (8.02 g, 47.2 mmol) and anhydrous EtOAc (300 mL). The mixture was stirred for 3 h at 50° C.

The solution from step (1) was added dropwise to the suspension from step (2) and the mixture stirred at reflux overnight. The mixture was cooled in an ice-bath and acidified with 4M HCl (140 mL) to pH=3. The mixture was allowed to warm to ambient temperature and the layers were separated. The aqueous layer was extracted with EtOAc, and the combined organic layers were washed with water, brine, and dried (Na₂SO₄). Concentration and purification by chromatography on silica gel gave compound P97 (4.3 g, 65%). ¹H NMR (400 MHz, DMSO-d₆), δ: 7.22 (br. s, 2H), 4.20 (d, J=2.3 Hz, 2H), 4.18-4.07 (m, 2H), 3.85 (br. s, 6H), 3.78 (br. s, 3H), 1.19 (t, J=9.4 Hz, 3H).

### Preparation 98. Ethyl (2Z)-3-(dimethylamino)-2-(3,4,5-trimethoxybenzoyl)acrylate (P98)

A solution of compound P97 (4.3 g, 15.2 mmol) and N,N-dimethylformamide dimethyl acetal (20.2 mL, 152.0 mmol) was stirred at reflux overnight. The mixture was evaporated to dryness and the residue was purified by column chromatography on silica gel. Yield of compound P98: 5.1 g, 99%. ¹H NMR (400 MHz, DMSO-d₆), δ: 7.64 (s, 1H), 6.94 (s, 2H), 3.90 (q, J=7.1 Hz, 2H), 3.78 (s, 6H), 3.71 (s, 3H), 2.79 (br. s, 6H), 0.91 (t, J=7.1 Hz, 3H).

### Preparation 99. Ethyl 7-(3,4,5-trimethoxyphenyl)pyrazolo[1,5-a]pyrimidine-6-carboxylate (P99)

To a solution of compound P98 (5.1 g, 15.0 mmol) in 100 mL of EtOH, 1H-pyrazol-5-amine (1.5 g, 18.0 mmol) was added. The mixture was stirred at reflux overnight. The mixture was evaporated, and the residue was purified by column chromatography on silica gel. Yield of compound P99: 3.85 g, 71%. ¹H NMR (400 MHz, DMSO-d₆), δ: 8.92 (s, 1H), 8.32 (d, J=2.2 Hz, 1H), 7.14-6.61 (m, 3H), 4.33-3.95 (m, 2H), 3.75 (d, J=3.2 Hz, 9H), 1.28-0.71 (m, 3H).

### Preparation 100. 7-(3,4,5-Trimethoxyphenyl)pyrazolo[1,5-a]pyrimidine-6-carboxylic acid (P100)

A mixture of compound P98 (3.85 g, 10.0 mmol) and KOH (1.2 g, 21.0 mmol) in 100 mL of EtOH and 20 mL of H₂O was stirred at 50° C. for 3 h and evaporated. The residue was diluted with water and acidified with 6 M HCl to pH=3-4. The formed solid was filtered off and dried. Yield of compound P100: 3.4 g, 96%. LCMS ESI (m/z): 330.5 ([M+1]⁺).

### Synthesis of 3-(2-{4-[2-(2,6-Dioxopiperidin-3-yl)-1-oxo-2,3-dihydro-1H-isoindol-5-yl]piperazin-1-yl}ethoxy)propanoic acid (P103)

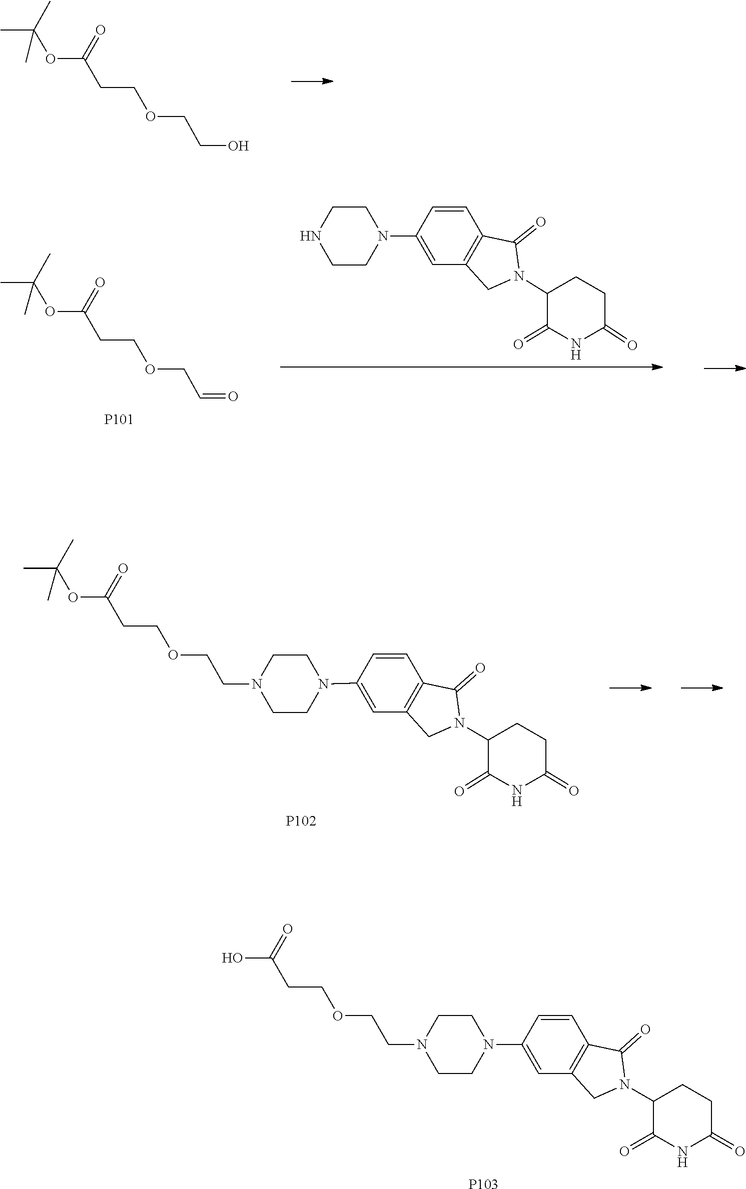

### Preparation 101. tert-Butyl 3-(2-oxoethoxy)propanoate (P101)

A solution of dimethyl sulfoxide (5.3 g, 67.8 mmol) in DCM (50 mL) was added to a stirred solution of oxalyl chloride (4.3 g, 33.9 mmol) in DCM (20 mL) at −70° C. A solution of tert-butyl 3-(2-hydroxyethoxy)propanoate (5.9 g, 30.8 mmol) in DCM (10 mL) was added dropwise to the above prepared solution over a period of 15 min and the mixture was stirred for an additional 3 h. Triethylamine (15.6 g, 154 mmol) was added dropwise with stirring for 15 min, and the whole was allowed to warm to rt. The mixture was diluted with DCM, washed saturated NaHCO₃ (aq.), dried with Na₂SO₄ and evaporated. The residue was used on the next step without additional purification.

### Preparation 102. tert-Butyl 3-(2-{4-[2-(2,6-dioxopiperidin-3-yl)-1-oxo-2,3-dihydro-1H-isoindol-5-yl]piperazin-1-yl}ethoxy)propanoate (P102)

A solution of tert-butyl 3-(2-oxoethoxy)propanoate P101 (0.5 g, 2.7 mmol) and 3-(1-oxo-5-piperazin-1-yl-1,3-dihydro-2H-isoindol-2-yl)piperidine-2,6-dione (0.85 g, 2.7 mmol) in 20 mL od DCM was stirred for 1 h at ambient temperature. STAB (1.7 g, 8.1 mmol) was added, and the mixture was stirred for 15 h at ambient temperature. The mixture was quenched with sat aq. NaHCO₃, extracted with DCM, the combined organic extracts were dried with Na₂SO₄ and evaporated. The residue was purified by column chromatography on silica gel eluting with EtOAc-Hx, 30 →100% giving tert-butyl 3-(2-{4-[2-(2,6-dioxopiperidin-3-yl)-1-oxo-2,3-dihydro-1H-isoindol-5-yl]piperazin-1-yl}ethoxy)propanoate P102 (0.99 g, 75%). LCMS ESI (m/z): 501.5 ([M+1]⁺).

### Preparation 103. 3-(2-{4-[2-(2,6-Dioxopiperidin-3-yl)-1-oxo-2,3-dihydro-1H-isoindol-5-yl]piperazin-1-yl}ethoxy)propanoic acid (P103)

To a solution of tert-butyl 3-(2-{4-[2-(2,6-dioxopiperidin-3-yl)-1-oxo-2,3-dihydro-1H-isoindol-5-yl]piperazin-1-yl}ethoxy)propanoate P102 (0.99 g, 2.0 mmol) in 20 mL of dioxane, 10 mL of 3M HCl in dioxane was added and the mixture was stirred at ambient temperature for 15 h. The solvents were evaporated, the residue was washed with Et₂O and dried giving 3-(2-{4-[2-(2,6-dioxopiperidin-3-yl)-1-oxo-2,3-dihydro-1H-isoindol-5-yl]piperazin-1-yl}ethoxy)propanoic acid P103 as hydrochloride (0.84 g, 95%). LCMS ESI (m/z): 445.5 ([M+1]⁺).

### Synthesis of N-{6-[5-(Aminomethyl)-1,2,4-oxadiazol-3-yl]-5-methylpyridin-3-yl}-N′-[7-(3,4,5-trimethoxyphenyl)pyrazolo[1,5-a]pyrimidin-6-yl]urea (P107)

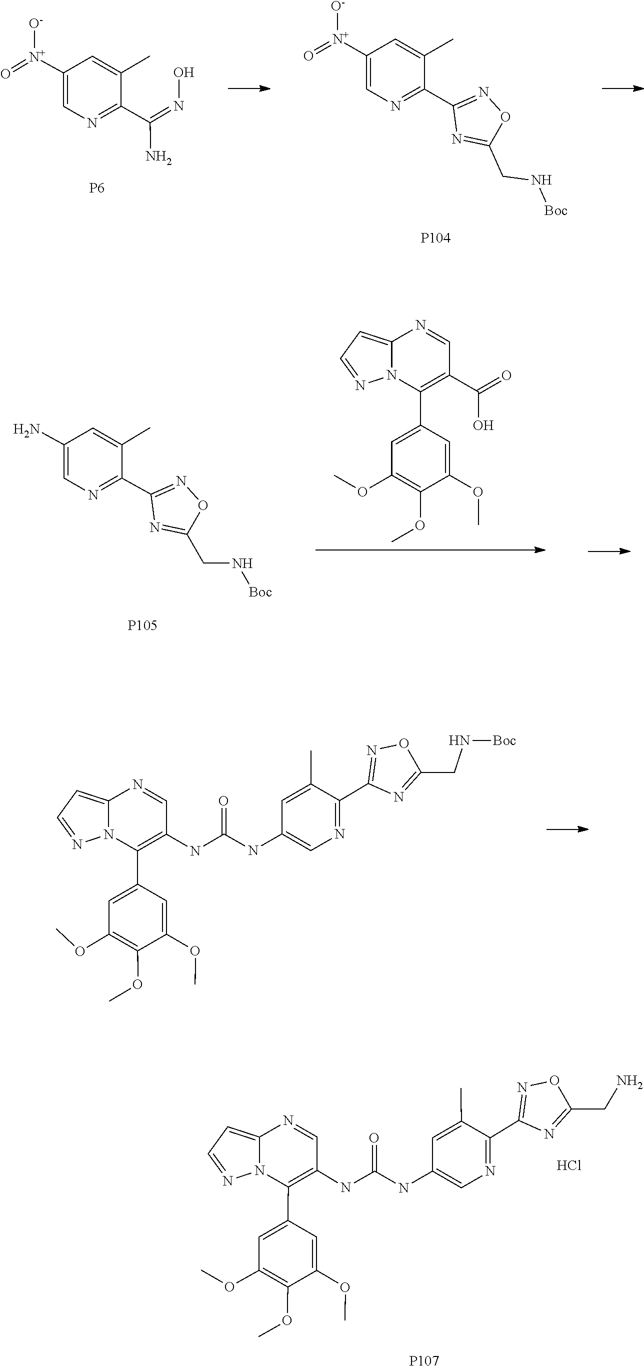

### Preparation 104. tert-Butyl {[3-(3-methyl-5-nitropyridin-2-yl)-1,2,4-oxadiazol-5-yl]methyl}carbamate (P104)

To a solution of N-Boc-glycine (1.93 g, 11 mmol) in DMAA (20 ml) was added CDI (2.14 g, 13.2 mmol), and solution was stirred at 50° C. for 30 min, then N-hydroxy-3-methyl-5-nitropyridine-2-carboximidamide P6 (2.14 g, 11 mmol) was added. The mixture was stirred at 50° C. for 2 h, and at 90° C. overnight, washed with water (200 ml), extracted with DCM (2×50 ml), organic phase was concentrated, and residue was purified by column chromatography on silica gel eluting with ethyl acetate/DCM to yield the compound P104: 1.1 g, 43%. LCMS ESI (m/z): 336.5 ([M+1]⁺).

### Preparation 105. tert-Butyl {[3-(3-methyl-5-aminopyridin-2-yl)-1,2,4-oxadiazol-5-yl]methyl}carbamate (P105)

To solution of tert-butyl {[3-(3-methyl-5-nitropyridin-2-yl)-1,2,4-oxadiazol-5-yl]methyl}carbamate P104 (1.1 g, 4.7 mmol) in MeOH (30 ml) Raney Nickel was added. The reaction mixture was stirred at hydrogen atmosphere for 2 h (TLC monitoring). After reaction was complete, the mixture was filtered, methanol was evaporated to dryness to yield Compound P105 (0.96 g, 100%). ¹H NMR (400 MHz, DMSO-d₆), δ: 7.89 (d, J=2.3 Hz, 1H), 7.69 (s, 1H), 6.82 (t, J=2.0 Hz, 1H), 5.82 (s, 2H), 4.45 (d, J=6.0 Hz, 2H), 2.36 (s, 3H), 2.07 (s, 2H), 1.40 (s, 9H).

### Preparation 106. tert-Butyl [(3-{3-methyl-5-[({[7-(3,4,5-trimethoxyphenyl)pyrazolo[1,5-a]pyrimidin-6-yl]amino}carbonyl)amino]pyridin-2-yl}-1,2,4-oxadiazol-5-yl)methyl]carbamate (P106)

To a solution of 7-(3,4,5-trimethoxyphenyl)pyrazolo[1,5-a]pyrimidine-6-carboxylic acid (329 mg, 1 mmol) in DMAA (8 ml) were added TEA (404 mg, 4 mmol) and the mixture was stirred at ambient temperature. DPPA (413 mg, 1.5 mmol) was added and the mixture was stirred at ambient temperature for 6 h, then tert-butyl {[3-(3-methyl-5-aminopyridin-2-yl)-1,2,4-oxadiazol-5-yl]methyl}carbamate P105 (204 mg, 1 mmol) was added and mixture was stirred at ambient temperature for 18 h. DMAA was evaporated to dryness, and residue was crystallized from EtOH giving Compound P106 (169 mg, 32%) as beige solid.

### Preparation 107. N-{6-[5-(Aminomethyl)-1,2,4-oxadiazol-3-yl]-5-methylpyridin-3-yl}-N-[7-(3,4,5-trimethoxyphenyl)pyrazolo[1,5-a]pyrimidin-6-yl]urea (P107)

N-{6-[5-(Aminomethyl)-1,2,4-oxadiazol-3-yl]-5-methylpyridin-3-yl}-N-[7-(3,4,5-trimethoxyphenyl)pyrazolo[1,5-a]pyrimidin-6-yl]urea P106 (169 mg, 0.32 mmol) was added to 3 M solution of HCl in dioxane (10 ml). The mixture was stirred at ambient temperature for 15 h, then evaporated to dryness, giving hydrochloride of N-{6-[5-(aminomethyl)-1,2,4-oxadiazol-3-yl]-5-methylpyridin-3-yl}-N-[7-(3,4,5-trimethoxyphenyl)pyrazolo[1,5-a]pyrimidin-6-yl]urea P107 (149 mg, 100%). LCMS ESI (m/z): 532.4 ([M+1]⁺).

### Synthesis of 1-[2-[2-(2-azidoethoxy)ethoxy]ethyl]-3-[2-(2,6-dioxo-3-piperidyl)-1-oxo-isoindolin-4-yl]urea (P111)

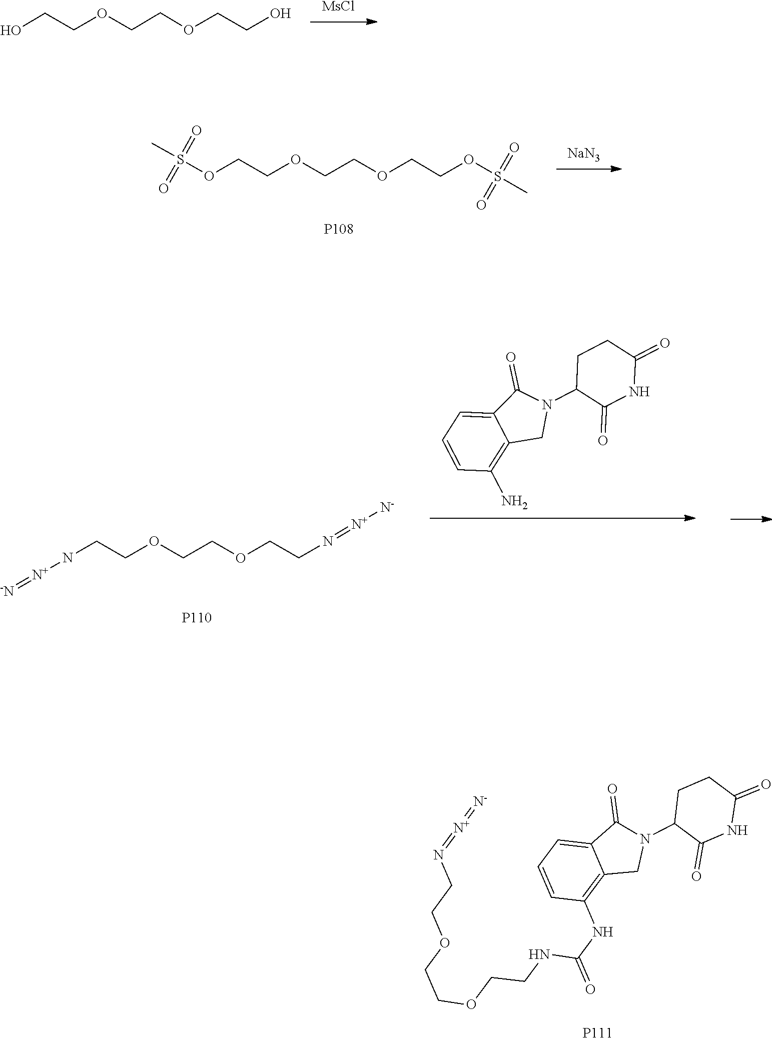

### Preparation 108. 1-Methanesulfonyloxy-2-[2-(2-methanesulfonylethoxy)ethoxy]ethane (P108)

MsCl (76.6 ml, 465 mmol) was added dropwise to a solution of 2,2′-[ethane-1,2-diylbis(oxy)]diethanol (20.0 g, 133 mmol) and Et₃N (21.6 ml, 279 mmol) in DCM (300 ml) at 0° C. Reaction mixture stirred at ambient temperature for 1 h. After reaction completed, the organic phase was washed with water and brine, dried over Na₂SO₄, and evaporated under reduced pressure. The residue was pure compound P108 (29.0 g, 71%). ¹H NMR (400 MHz, CDCl₃), δ: 4.55-4.24 (m, 4H), 3.85-3.73 (m, 4H), 3.68 (s, 4H), 3.07 (s, 6H).

### Preparation 109. 1-Azido-2-[2-(2-azidoethoxy)ethoxy]ethane (P109)

To a solution of compound P108 (29.0 g, 95 mmol) in DMF (200 ml) was added sodium azide (18.5 g, 285 mmol). The reaction mixture was stirred at 70° C. for 24 h. The reaction mixture was diluted with water and extracted with Et₂O; the organic phase was dried over Na₂SO₄ and evaporated under reduced pressure. The crude product P109 was used in the further steps without any additional purification. Yield of P109: 18.0 g (95%). ¹H NMR (400 MHz, CDCl₃), δ: 3.70-3.58 (m, 8H), 3.33 (t, J=4.9 Hz, 4H).

### Preparation 110. {2-[2-(2-Azidoethoxy)ethoxy]ethyl}amine (P110)

To a solution of compound P109 (18.0 g, 95 mmol) in Et₂O (225 ml) and 1M HCl (225 ml) was added triphenylphosphine (12.3 g, 48 mmol). Reaction mixture was stirred at ambient temperature for 18 h. The aqueous layer was separated and washed with ether. The aqueous one was alkalized with 1N NaOH (pH˜11) and extracted with DCM. The organic layer was washed with brine, dried over sodium sulfate, and evaporated. The crude product P110 used to next step without purification. The yield of P110 was 17.0 g (99%). ¹H NMR (400 MHz, CDCl₃), δ: 3.73-3.59 (m, 8H), 3.54 (dd, J=13.6, 8.4 Hz, 2H), 3.46-3.35 (m, 2H), 1.90 (br. s, 2H).

### Preparation 111. 1-[2-[2-(2-azidoethoxy)ethoxy]ethyl]-3-[2-(2,6-dioxo-3-piperidyl)-1-oxo-isoindolin-4-yl]urea (P111)

Triphosgene (1.14 g, 3.84 mmol) was dissolved in DCM (20 ml). After cooling to −10° C., {2-[2-(2-azidoethoxy)ethoxy]ethyl}amine (4) (2.0 g, 11.5 mmol) was slowly added dropwise, and stirred at −10° C. for 0.5 h. Then, Et₃N (2.34 g, 23.0 mmol) was added and reacted for an additional 1 h. The reaction solution was slowly added dropwise to a solution of 3-(4-amino-1-oxo-1,3-dihydro-2H-isoindol-2-yl)piperidine-2,6-dione (2.0 g, 7.7 mmol) in N,N-dimethylacetamide (20 ml), and then reacted at 65° C. for 1 h. The reaction solution was poured into iced water, the aqueous phase was extracted with EtOAc, and the organic phase was washed with water and brine, dried and concentrated. The crude product was separated by column chromatography eluting with EtOAc-hexane to afford Compound P111 (2.48 g, 70%). LCMS ESI (m/z): 460.4 ([M+1]⁺), 329.4, 260.3.

### Synthesis of N-(7-Cyclopentylpyrazolo[1,5-a]pyrimidin-6-yl)-N′-{5-methyl-6-[5-({3-[(5-oxopentyl)oxy]propoxy}methyl)-1,2,4-oxadiazol-3-yl]pyridin-3-yl}urea (P118)

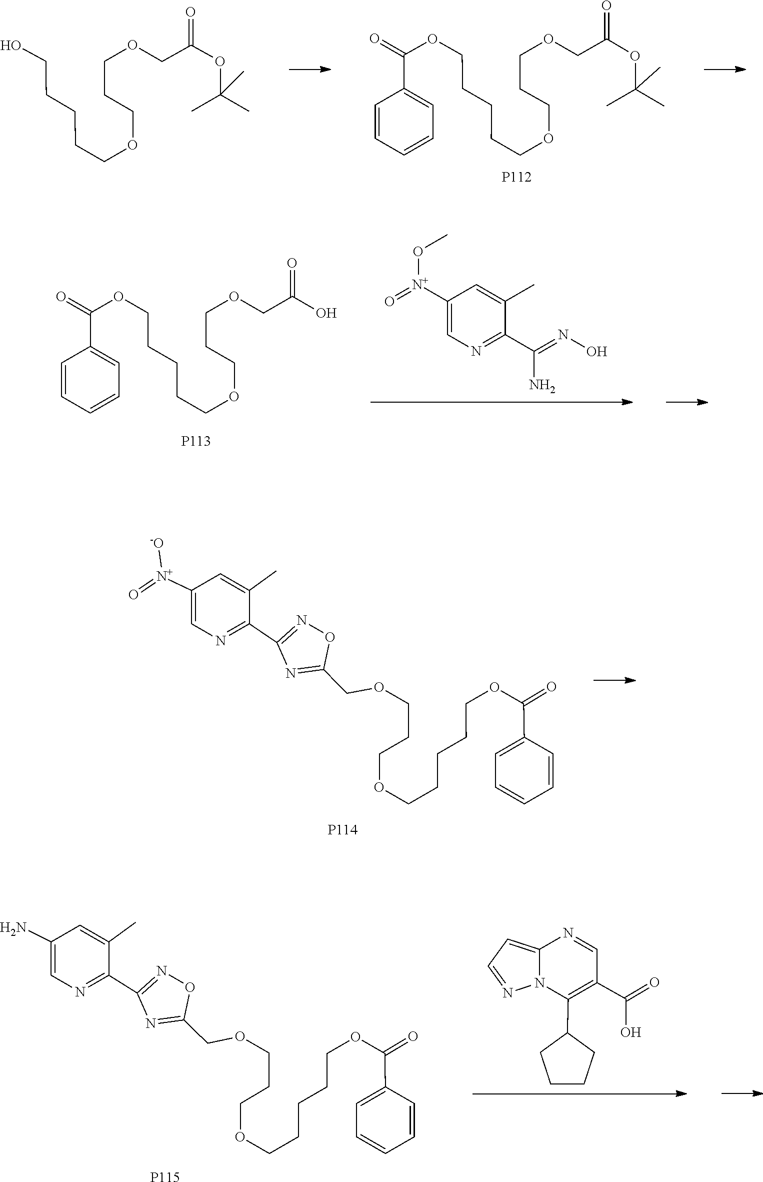

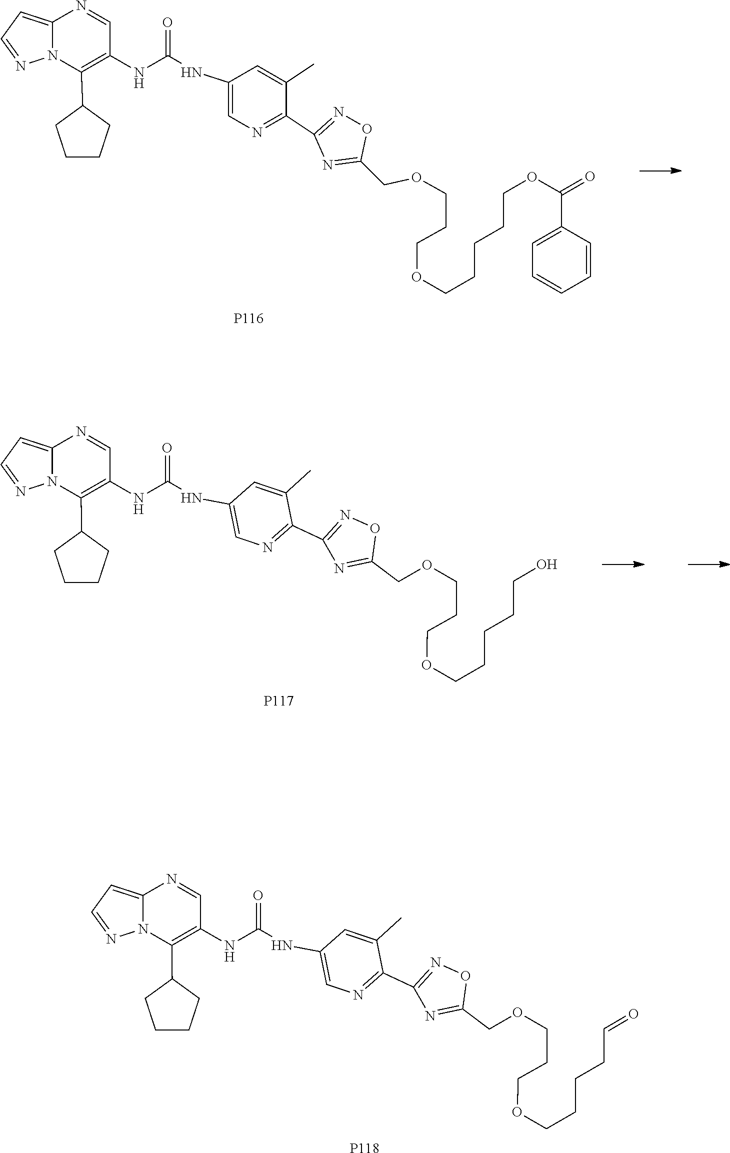

### Preparation 112. 5-[3-(2-tert-Butoxy-2-oxoethoxy)propoxy]pentyl benzoate (P112)

Et₃N (0.547 g, 5.4 mmol) was added to a solution of tert-butyl {3-[(5-hydroxypentyl)oxy]propoxy}acetate (0.498 g, 1.8 mmol) in DCM. The mixture was cooled to 0° C. and benzoyl chloride (0.547 g, 5.4 mmol) was added by dropwise at this temperature. Then the reaction mixture was stirred for 12 hours at rt. When reaction was completed (TLC monitoring), the reaction mixture was washed with water and evaporated. The product was purified by column chromatography on silica gel. Eluent—hexane/ethyl acetate, 20:1. 5-[3 (2-tert-Butoxy-2-oxoethoxy)propoxy]pentyl benzoate P112 was obtained with a yield of 0.426 g, 62%. ¹H NMR (400 MHz, CDCl₃), δ: 8.09-8.01 (m, 2H), 7.57 (t, J=7.4 Hz, 1H), 7.49-7.39 (m, 2H), 4.34 (t, J=6.6 Hz, 2H), 3.95 (s, 2H), 3.61 (t, J=6.4 Hz, 2H), 3.54 (t, J=6.4 Hz, 2H), 3.46 (t, J=6.4 Hz, 2H), 1.90 (p, J=6.3 Hz, 2H), 1.80 (dd, J=14.6, 7.0 Hz, 2H), 1.73-1.62 (m, 2H), 1.60-1.51 (m, 2H), 1.50 (d, J=6.1 Hz, 9H).

### Preparation 113. (3-{[5-(Benzoyloxy)pentyl]oxy}propoxy)acetic acid (P113)

5-[3-(2-tert-Butoxy-2-oxoethoxy)propoxy]pentyl benzoate P112 (0.818 g, 2.15 mmol) was dissolved in a mixture of 10 ml DCM and TFA (2.45 g, 21.5 mmol). The solution was stirred at rt for 15 h. The reaction mixture was washed with water and evaporated. (3-{[5-(Benzoyloxy)pentyl]oxy}propoxy)acetic acid P113 (0.694 g) was obtained with a yield of 99%. ¹H NMR (400 MHz, CDCl₃), δ: 8.05 (d, J=7.0 Hz, 2H), 7.56 (d, J=7.4 Hz, 1H), 7.45 (t, J=6.8 Hz, 2H), 4.34 (d, J=4.8 Hz, 2H), 4.09 (s, 2H), 3.68 (s, 2H), 3.61 (s, 2H), 3.49 (d, J=4.7 Hz, 2H), 1.90 (br. s, 2H), 1.80 (d, J=6.3 Hz, 2H), 1.68 (d, J=6.0 Hz, 2H), 1.52 (d, J=7.3 Hz, 2H).

### Preparation 114. 5-(3-{[3-(3-Methyl-5-nitropyridin-2-yl)-1,2,4-oxadiazol-5-yl]methoxy}propoxy)pentyl benzoate (P114)

To a solution of (3-{[5-(benzoyloxy)pentyl]oxy}propoxy)acetic acid (0.694 g, 2.14 mmol) in 30 ml of DCM, TEA (0.866 g, 8.56 mmol) was added. The solution was stirred for 5 min, then TBTU (0.825 g, 2.57 mmol) was added, and the mixture was stirred for 10 min. Compound P6 (0.42 g, 2.14 mmol) was added into the reaction flask and the mixture was stirred for 12 h at rt. After completion of the reaction as indicated by TLC, the reaction mixture was concentrated under vacuum, dissolved in ethyl acetate, and washed with water and evaporated. The residue was used for the next step without additional purification. It was dissolved in 50 ml of 1,4-dioxane. The solution was refluxed for 200 h. After completion of the reaction as indicated by LCMS, the reaction mixture was concentrated under vacuum. The residue was purified on silica gel eluting with EtOAc/hexane=1/4, giving Compound P114 (0.358 g). The yield was 35% for two steps. LCMS ESI (m/z): 485.8 ([M+H]⁺).

### Preparation 115. 5-(3-{[3-(5-Amino-3-methylpyridin-2-yl)-1,2,4-oxadiazol-5-yl]methoxy}propoxy)pentyl benzoate (P115)

Compound P114 (0.358 g, 0.739 mmol) was dissolved in 30 mL of a solvent consisting of ethanol and acetic acid in equal amounts. The mixture was heated to 40° C. and five equivalents of Fe powder (0.206 g, 3.7 mmol) were added. The stirring was continued at this temperature until the starting substance was completely converted as indicated by TLC. The mixture was cooled down to ambient temperature and passed through Celite. The filtrate was evaporated, and the residue was dissolved in ethyl acetate and washed with water and concentrated. Then the mixture was dissolved in methylene chloride, washed with an aqueous solution of sodium bicarbonate, dried over sodium sulfate and concentrated, giving compound P115 (0.317 g, 94%). LCMS ESI (m/z): 455.4 ([M+H]⁺).

### Preparation 116. 5-[3-({3-[5-({[(7-Cyclopentylpyrazolo[1,5-a]pyrimidin-6-yl)amino]carbonyl}amino)-3-methylpyridin-2-yl]-1,2,4-oxadiazol-5-yl}methoxy)propoxy]pentyl benzoate (P116)

Et₃N (0.282 g, 2.79 mmol) was added to a suspension of 7-cyclopentylpyrazolo[1,5-a]pyrimidine-6-carboxylic acid P36 (0.161 g, 0.697 mmol) in 30 mL of toluene. The mixture was stirred for 5 min. Then DPPA (0.23 g, 0.836 mmol) was added, and the reaction mixture was stirred for 60 min at rt. After this Compound P115 (0.317 g, 0.697 mmol) was added and the mixture was refluxed for 12 h. Reaction was monitored by LCMS. The mixture was quenched with water, separated toluene was dried with Na₂SO₄ and evaporated. The residue was purified on silica gel eluting with hexane/ethyl acetate, 1:2, giving Compound P116 (0.237 g, 50%). LCMS ESI (m/z): 683.5 ([M+H]⁺).

### Preparation 117. N-(7-Cyclopentylpyrazolo[1,5-a]pyrimidin-6-yl)-N′-{6-[5-({3-[(5-hydroxypentyl)oxy]propoxy}methyl)-1,2,4-oxadiazol-3-yl]-5-methylpyridin-3-yl}urea (P117)

Potassium carbonate (83.8 mg, 0.606 mmol) was added to a solution of Compound P116 (207 mg, 0.303 mmol) in MeOH (20 ml). The mixture was stirred for 12 h at 50° C. Reaction was monitored by LCMS. The mixture was quenched with water and product was extracted with ethyl acetate, organic layer was dried with Na₂SO₄ and concentrated. The yield of P117: 170 mg, 97%. LCMS ESI (m/z): 579.5 ([M+H]⁺).

### Preparation 118. N-(7-Cyclopentylpyrazolo[1,5-a]pyrimidin-6-yl)-N′-{5-methyl-6-[5-({3-[(5-oxopentyl)oxy]propoxy}methyl)-1,2,4-oxadiazol-3-yl]pyridin-3-yl}urea (P118)

Dess-Martin periodinane (0.167 g, 0.395 mmol) was added to a solution of Compound P118 (0.109 mg, 0.188 mmol) in DCM (5 ml). The mixture was stirred for 3 h at rt. Reaction was monitored by LCMS. After completion of the reaction, the reaction mass was washed with an aqueous solution of sodium bicarbonate. The organic layer was concentrated, and residue was used immediately for the next step without additional purification. LCMS ESI (m/z): 577.8 ([M+H]⁺).

In the Table 3 presented certain examples of key intermediates.

TABLE 3
Certain examples of key intermediates.

| | | [MH]⁺ | [MH]⁺ |
|---|---|---|---|
| # | IUPAC Name | Calc. | Found |
| P4 | 7-tetrahydrofuran-2-ylpyrazolo[1,5-a]pyrimidine-6- | 234.088 | 234 |
| | carboxylic acid | | |
| P36 | 7-cyclopentylpyrazolo[1,5-a]pyrimidine-6- | 232.109 | 232 |
| | carboxylic acid | | |
| P100 | 7-(3,4,5-trimethoxyphenyl)pyrazolo[1,5- | 330.109 | 330 |
| | a]pyrimidine-6-carboxylic acid | | |
| P64 | tert-butyl 2-(4-azidobutoxy)acetate | — | — |
| P68 | 2-(4-benzoyloxybutoxy)acetic acid | — | — |
| P75 | 3-(2-benzoyloxyethoxy)propanoic acid | — | — |
| P84 | 5-methoxy-3,3-dimethyl-5-oxo-pentanoic acid | — | — |
| P9 | ethyl 6-[3-(5-amino-3-methyl-2-pyridyl)-1,2,4- | 319.177 | 319 |
| | oxadiazol-5-yl]hexanoate | | |
| P39 | ethyl 4-[3-(5-amino-3-methyl-2-pyridyl)-1,2,4- | 291.146 | 291 |
| | oxadiazol-5-yl]butanoate | | |
| P44 | ethyl 3-[3-(5-amino-3-methyl-2-pyridyl)-1,2,4- | 277.13 | 277 |
| | oxadiazol-5-yl]propanoate | | |
| P49 | ethyl 5-[3-(5-amino-3-methyl-2-pyridyl)-1,2,4- | 305.161 | 305 |
| | oxadiazol-5-yl]pentanoate | | |
| P70 | 4-[[3-(5-amino-3-methyl-2-pyridyl)-1,2,4- | 383.172 | 383 |
| | oxadiazol-5-yl]methoxy]butyl benzoate | | |
| P78 | 2-[2-[3-(5-amino-3-methyl-2-pyridyl)-1,2,4- | 369.156 | 369 |
| | oxadiazol-5-yl]ethoxy]ethyl benzoate | | |
| P11 | 6-[3-[3-methyl-5-[(7-tetrahydrofuran-2- | 521.226 | 521 |
| | ylpyrazolo[1,5-a]pyrimidin-6-yl)carbamoylamino]- | | |
| | 2-pyridyl]-1,2,4-oxadiazol-5-yl]hexanoic acid | | |
| P18 | 6-[3-[3-chloro-5-[(7-cyclopentylpyrazolo[1,5- | 539.192 | 539 |
| | a]pyrimidin-6-yl)carbamoylamino]-2-pyridyl]- | | |
| | 1,2,4-oxadiazol-5-yl]hexanoic acid | | |
| P26 | 6-[5-[5-[(7-cyclopentylpyrazolo[1,5-a]pyrimidin-6- | 519.247 | 519 |
| | yl)carbamoylamino]-3-methyl-2-pyridyl]-1,3,4- | | |
| | oxadiazol-2-yl]hexanoic acid | | |
| P32 | 7-[4-[5-[(7-cyclopentylpyrazolo[1,5-a]pyrimidin-6- | 532.278 | 532 |
| | yl)carbamoylamino]-3-methyl-2-pyridyl]triazol-1- | | |
| | yl]heptanoic acid | | |
| P41 | 4-[3-[5-[(7-cyclopentylpyrazolo[1,5-a]pyrimidin-6- | 491.216 | 491 |
| | yl)carbamoylamino]-3-methyl-2-pyridyl]-1,2,4- | | |
| | oxadiazol-5-yl]butanoic acid | | |
| P46 | 3-[3-[5-[(7-cyclopentylpyrazolo[1,5-a]pyrimidin-6- | 477.2 | 477 |
| | yl)carbamoylamino]-3-methyl-2-pyridyl]-1,2,4- | | |
| | oxadiazol-5-yl]propanoic acid | | |
| P53 | 1-(7-cyclopentylpyrazolo[1,5-a]pyrimidin-6-yl)-3- | 489.236 | 489 |
| | [5-methyl-6-[5-(5-oxopentyl)-1,2,4-oxadiazol-3-yl]- | | |
| | 3-pyridyl]urea | | |
| P55 | 1-(7-cyclopentylpyrazolo[1,5-a]pyrimidin-6-yl)-3- | 461.205 | 461 |
| | [5-methyl-6-[5-(3-oxopropyl)-1,2,4-oxadiazol-3-yl]- | | |
| | 3-pyridyl]urea | | |
| P56 | 1-(3-chloro-7-cyclopentyl-pyrazolo[1,5- | 495.166 | 495 |
| | a]pyrimidin-6-yl)-3-[5-methyl-6-[5-(3-oxopropyl)- | | |
| | 1,2,4-oxadiazol-3-yl]-3-pyridyl]urea | | |
| P58 | 1-(3-chloro-7-cyclopentyl-pyrazolo[1,5- | 509.182 | 509 |
| | a]pyrimidin-6-yl)-3-[5-methyl-6-[5-(4-oxobutyl)- | | |
| | 1,2,4-oxadiazol-3-yl]-3-pyridyl]urea | | |
| P59 | 1-(7-cyclopentylpyrazolo[1,5-a]pyrimidin-6-yl)-3- | 475.221 | 475 |
| | [5-methyl-6-[5-(4-oxobutyl)-1,2,4-oxadiazol-3-yl]- | | |
| | 3-pyridyl]urea | | |
| P62 | 6-[3-[5-[(7-cyclopentylpyrazolo[1,5-a]pyrimidin-6- | 519.247 | 519 |
| | yl)carbamoylamino]-3-methyl-2-pyridyl]-1,2,4- | | |
| | oxadiazol-5-yl]hexanoic acid | | |
| P66 | 2-[4-[4-[5-[(7-cyclopentylpyrazolo[1,5-a]pyrimidin- | 534.258 | 534 |
| | 6-yl)carbamoylamino]-3-methyl-2-pyridyl]triazol-1- | | |
| | yl]butoxy]acetic acid | | |
| P73 | 1-(7-cyclopentylpyrazolo[1,5-a]pyrimidin-6-yl)-3- | 505.231 | 505 |
| | [5-methyl-6-[5-(4-oxobutoxymethyl)-1,2,4- | | |
| | oxadiazol-3-yl]-3-pyridyl]urea | | |
| P81 | 1-(7-cyclopentylpyrazolo[1,5-a]pyrimidin-6-yl)-3- | 491.216 | 491 |
| | [5-methyl-6-[5-[2-(2-oxoethoxy)ethyl]-1,2,4- | | |
| | oxadiazol-3-yl]-3-pyridyl]urea | | |
| P82 | 6-[3-[5-[(5-cyclopentylimidazo[1,2-a]pyrimidin-6- | 519.247 | 519 |
| | yl)carbamoylamino]-3-methyl-2-pyridyl]-1,2,4- | | |
| | oxadiazol-5-yl]hexanoic acid | | |
| P88 | 4-[3-[5-[(7-cyclopentylpyrazolo[1,5-a]pyrimidin-6- | 519.247 | 519 |
| | yl)carbamoylamino]-3-methyl-2-pyridyl]-1,2,4- | | |
| | oxadiazol-5-yl]-3,3-dimethyl-butanoic acid | | |
| P96 | 1-[5-chloro-6-[5-(4-oxobutyl)-1,2,4-oxadiazol-3- | 495.166 | 495 |
| | yl]-3-pyridyl]-3-(7-cyclopentylpyrazolo[1,5- | | |
| | a]pyrimidin-6-yl)urea | | |
| P118 | 1-(7-cyclopentylpyrazolo[1,5-a]pyrimidin-6-yl)-3- | 577.289 | 578 |
| | [5-methyl-6-[5-[3-(5-oxopentoxy)propoxymethyl]- | | |
| | 1,2,4-oxadiazol-3-yl]-3-pyridyl]urea | | |
| P107 | 1-[6-[5-(aminomethyl)-1,2,4-oxadiazol-3-yl]-5- | 532.206 | 532 |
| | methyl-3-pyridyl]-3-[7-(3,4,5- | | |
| | trimethoxyphenyl)pyrazolo[1,5-a]pyrimidin-6- | | |
| | yl]urea | | |
| P103 | 3-[2-[4-[2-(2,6-dioxo-3-piperidyl)-1-oxo-isoindolin- | 445.209 | 445 |
| | 5-yl]piperazin-1-yl]ethoxy]propanoic acid | | |
| P111 | 1-[2-[2-(2-azidoethoxy)ethoxy]ethyl]-3-[2-(2,6- | 460.194 | 460 |
| | dioxo-3-piperidyl)-1-oxo-isoindolin-4-yl]urea | | |

### Examples of the Final Compound

In the Table 4 presented certain non-limiting examples of the compound of Formula (A).

TABLE 4
Selected examples of the compound of Formula (A)

| | | [MH]⁺ | [MH]⁺ |
|---|---|---|---|
| # | IUPAC Name | Calc. | Found |
| 1 | 3-[2-[4-[2-(2,6-dioxo-3-piperidyl)-1-oxo-isoindolin-5- | 958.40 | 958 |
| | yl]piperazin-1-yl]ethoxy]-N-[[3-[3-methyl-5-[[7-(3,4,5- | | |
| | trimethoxyphenyl)pyrazolo[1,5-a]pyrimidin-6- | | |
| | yl]carbamoylamino]-2-pyridyl]-1,2,4-oxadiazol-5- | | |
| | yl]methyl]propenamide | | |
| 2 | 1-[6-[5-[6-[4-[2-(2,6-dioxo-3-piperidyl)-1-oxo-isoindolin-5- | 831.37 | 831 |
| | yl]piperazin-1-yl]-6-oxo-hexyl]-1,2,4-oxadiazol-3-yl]-5- | | |
| | methyl-3-pyridyl]-3-(7-tetrahydrofuran-2-ylpyrazolo[1,5- | | |
| | a]pyrimidin-6-yl)urea | | |
| 3 | 1-[5-chloro-6-[5-[6-[4-[2-(2,6-dioxo-3-piperidyl)-1-oxo- | 849.34 | 849 |
| | isoindolin-5-yl]piperazin-1-yl]-6-oxo-hexyl]-1,2,4-oxadiazol-3- | | |
| | yl]-3-pyridyl]-3-(7-cyclopentylpyrazolo[1,5-a]pyrimidin-6- | | |
| | yl)urea | | |
| 4 | 1-(7-cyclopentylpyrazolo[1,5-a]pyrimidin-6-yl)-3-[6-[5-[6-[4- | 829.39 | 829 |
| | [2-(2,6-dioxo-3-piperidyl)-1-oxo-isoindolin-5-yl]piperazin-1- | | |
| | yl]-6-oxo-hexyl]-1,2,4-oxadiazol-3-yl]-5-methyl-3-pyridyl]urea | | |
| 5 | 1-(7-cyclopentylpyrazolo[1,5-a]pyrimidin-6-yl)-3-[6-[5-[6-[4- | 829.39 | 829 |
| | [2-(2,6-dioxo-3-piperidyl)-1-oxo-isoindolin-5-yl]piperazin-1- | | |
| | yl]-6-oxo-hexyl]-1,3,4-oxadiazol-2-yl]-5-methyl-3-pyridyl]urea | | |
| 6 | 1-(7-cyclopentylpyrazolo[1,5-a]pyrimidin-6-yl)-3-[6-[1-[7-[4- | 842.42 | 842 |
| | [2-(2,6-dioxo-3-piperidyl)-1-oxo-isoindolin-5-yl]piperazin-1- | | |
| | yl]-7-oxo-heptyl]triazol-4-yl]-5-methyl-3-pyridyl]urea | | |
| 7 | 1-(7-cyclopentylpyrazolo[1,5-a]pyrimidin-6-yl)-3-[6-[1-[2-[2- | 820.36 | 820 |
| | [2-[[2-(2,6-dioxo-3-piperidyl)-1-oxo-isoindolin-4- | | |
| | yl]carbamoylamino]ethoxy]ethoxy]ethyl]triazol-4-yl]-5- | | |
| | methyl-3-pyridyl]urea | | |
| 8 | 1-(7-cyclopentylpyrazolo[1,5-a]pyrimidin-6-yl)-3-[6-[1-[4-[2- | 844.40 | 844 |
| | [4-[2-(2,6-dioxo-3-piperidyl)-1-oxo-isoindolin-5-yl]piperazin- | | |
| | 1-yl]-2-oxo-ethoxy]butyl]triazol-4-yl]-5-methyl-3-pyridyl]urea | | |
| 9 | 1-(5-cyclopentylimidazo[1,2-a]pyrimidin-6-yl)-3-[6-[5-[6-[4- | 829.39 | 829 |
| | [2-(2,6-dioxo-3-piperidyl)-1-oxo-isoindolin-5-yl]piperazin-1- | | |
| | yl]-6-oxo-hexyl]-1,2,4-oxadiazol-3-yl]-5-methyl-3-pyridyl]urea | | |
| 10 | 1-(7-cyclopentylpyrazolo[1,5-a]pyrimidin-6-yl)-3-[6-[5-[4-[4- | 829.39 | 829 |
| | [2-(2,6-dioxo-3-piperidyl)-1-oxo-isoindolin-5-yl]piperazin-1- | | |
| | yl]-2,2-dimethyl-4-oxo-butyl]-1,2,4-oxadiazol-3-yl]-5-methyl- | | |
| | 3-pyridyl]urea | | |
| 11 | 1-(7-cyclopentylpyrazolo[1,5-a]pyrimidin-6-yl)-3-[6-[5-[4-[4- | 817.39 | 817 |
| | [2-(2,6-dioxo-3-piperidyl)-1-oxo-isoindolin-5-yl]piperazin-1- | | |
| | yl]butoxymethyl]-1,2,4-oxadiazol-3-yl]-5-methyl-3- | | |
| | pyridyl]urea | | |
| 12 | 1-(7-cyclopentylpyrazolo[1,5-a]pyrimidin-6-yl)-3-[6-[5-[4-[4- | 801.36 | 801 |
| | [2-(2,6-dioxo-3-piperidyl)-1-oxo-isoindolin-5-yl]piperazin-1- | | |
| | yl]-4-oxo-butyl]-1,2,4-oxadiazol-3-yl]-5-methyl-3-pyridyl]urea | | |
| 13 | 1-(7-cyclopentylpyrazolo[1,5-a]pyrimidin-6-yl)-3-[6-[5-[3-[4- | 787.34 | 787 |
| | [2-(2,6-dioxo-3-piperidyl)-1-oxo-isoindolin-5-yl]piperazin-1- | | |
| | yl]-3-oxo-propyl]-1,2,4-oxadiazol-3-yl]-5-methyl-3- | | |
| | pyridyl]urea | | |
| 14 | 1-(7-cyclopentylpyrazolo[1,5-a]pyrimidin-6-yl)-3-[6-[5-[5-[4- | 815.37 | 815 |
| | [2-(2,6-dioxo-3-piperidyl)-1-oxo-isoindolin-5-yl]piperazin-1- | | |
| | yl]-5-oxo-pentyl]-1,2,4-oxadiazol-3-yl]-5-methyl-3- | | |
| | pyridyl]urea | | |
| 15 | 1-(7-cyclopentylpyrazolo[1,5-a]pyrimidin-6-yl)-3-[6-[5-[3-[5- | 889.45 | 889 |
| | [4-[2-(2,6-dioxo-3-piperidyl)-1-oxo-isoindolin-5-yl]piperazin- | | |
| | 1-yl]pentoxy]propoxymethyl]-1,2,4-oxadiazol-3-yl]-5-methyl- | | |
| | 3-pyridyl]urea | | |
| 16 | 1-(7-cyclopentylpyrazolo[1,5-a]pyrimidin-6-yl)-3-[6-[5-[2-[2- | 803.37 | 803 |
| | [4-[2-(2,6-dioxo-3-piperidyl)-1-oxo-isoindolin-5-yl]piperazin- | | |
| | 1-yl]ethoxy]ethyl]-1,2,4-oxadiazol-3-yl]-5-methyl-3- | | |
| | pyridyl]urea | | |
| 17 | 1-(7-cyclopentylpyrazolo[1,5-a]pyrimidin-6-yl)-3-[6-[5-[5-[4- | 801.40 | 801 |
| | [2-(2,6-dioxo-3-piperidyl)-1-oxo-isoindolin-5-yl]piperazin-1- | | |
| | yl]pentyl]-1,2,4-oxadiazol-3-yl]-5-methyl-3-pyridyl]urea | | |
| 18 | 1-(7-cyclopentylpyrazolo[1,5-a]pyrimidin-6-yl)-3-[6-[5-[3-[4- | 773.36 | 773 |
| | [2-(2,6-dioxo-3-piperidyl)-1-oxo-isoindolin-5-yl]piperazin-1- | | |
| | yl]propyl]-1,2,4-oxadiazol-3-yl]-5-methyl-3-pyridyl]urea | | |
| 19 | 1-(3-chloro-7-cyclopentyl-pyrazolo[1,5-a]pyrimidin-6-yl)-3-[6- | 807.33 | 807 |
| | [5-[3-[4-[2-(2,6-dioxo-3-piperidyl)-1-oxo-isoindolin-5- | | |
| | yl]piperazin-1-yl]propyl]-1,2,4-oxadiazol-3-yl]-5-methyl-3- | | |
| | pyridyl]urea | | |
| 20 | 1-(3-chloro-7-cyclopentyl-pyrazolo[1,5-a]pyrimidin-6-yl)-3-[6- | 821.34 | 821 |
| | [5-[4-[4-[2-(2,6-dioxo-3-piperidyl)-1-oxo-isoindolin-5- | | |
| | yl]piperazin-1-yl]butyl]-1,2,4-oxadiazol-3-yl]-5-methyl-3- | | |
| | pyridyl]urea | | |
| 21 | 1-(7-cyclopentylpyrazolo[1,5-a]pyrimidin-6-yl)-3-[6-[5-[4-[4- | 787.38 | 787 |
| | [2-(2,6-dioxo-3-piperidyl)-1-oxo-isoindolin-5-yl]piperazin-1- | | |
| | yl]butyl]-1,2,4-oxadiazol-3-yl]-5-methyl-3-pyridyl]urea | | |
| 22 | 1-(3-chloro-7-cyclopentyl-pyrazolo[1,5-a]pyrimidin-6-yl)-3-[5- | 841.29 | 841 |
| | chloro-6-[5-[4-[4-[2-(2,6-dioxo-3-piperidyl)-1-oxo-isoindolin- | | |
| | 5-yl]piperazin-1-yl]butyl]-1,2,4-oxadiazol-3-yl]-3-pyridyl]urea | | |
| 23 | 1-(7-cyclopentylpyrazolo[1,5-a]pyrimidin-6-yl)-3-[6-[5-[3-[4- | 805.33 | 805 |
| | [2-(2,6-dioxo-3-piperidyl)-6-fluoro-1,3-dioxo-isoindolin-5- | | |
| | yl]piperazin-1-yl]propyl]-1,2,4-oxadiazol-3-yl]-5-methyl-3- | | |
| | pyridyl]urea | | |
| 24 | 1-(3-chloro-7-cyclopentyl-pyrazolo[1,5-a]pyrimidin-6-yl)-3-[6- | 839.29 | 839 |
| | [5-[3-[4-[2-(2,6-dioxo-3-piperidyl)-6-fluoro-1,3-dioxo- | | |
| | isoindolin-5-yl]piperazin-1-yl]propyl]-1,2,4-oxadiazol-3-yl]-5- | | |
| | methyl-3-pyridyl]urea | | |
| 25 | N-[2-[[5-chloro-3-[[7-[(1S)-1-methoxyethyl]pyrazolo[1,5- | 832.33 | 832 |
| | a]pyrimidin-6-yl]carbamoylamino]-2-pyridyl]oxy]ethyl]-3-[2- | | |
| | [4-[2-(2,6-dioxo-3-piperidyl)-1-oxo-isoindolin-5-yl]piperazin- | | |
| | 1-yl]ethoxy]propanamide | | |
| 26 | N-[2-(2,6-dioxo-3-piperidyl)-1,3-dioxo-isoindolin-4-yl]-2-[2- | 890.32 | 890 |
| | [4-[3-methyl-5-[[7-(3,4,5-trimethoxyphenyl)pyrazolo[1,5- | | |
| | a]pyrimidin-6-yl]carbamoylamino]-2-pyridyl]piperazin-1-yl]-2- | | |
| | oxo-ethoxy]acetamide | | |
| 27 | 1-[6-[2-[2-[3-[4-[2-(2,6-dioxo-3-piperidyl)-1-oxo-isoindolin-5- | 920.41 | 920 |
| | yl]piperazin-1-yl]-3-oxo-propoxy]ethoxy]ethylamino]-5- | | |
| | methyl-3-pyridyl]-3-[7-(3,4,5-trimethoxyphenyl)pyrazolo[1,5- | | |
| | a]pyrimidin-6-ylurea | | |
| 28 | N-[2-[2-[3-[4-[2-(2,6-dioxo-3-piperidyl)-1-oxo-isoindolin-5- | 1014.42 | 1014 |
| | yl]piperazin-1-yl]-3-oxo-propoxy]ethoxy]ethyl]-1-[3-methyl-5- | | |
| | [[7-(3,4,5-trimethoxyphenyl)pyrazolo[1,5-a]pyrimidin-6- | | |
| | yl]carbamoylamino]-2-pyridyl]pyrazole-4-carboxamide | | |
| 29 | 1-[6-[2-[2-[2-[3-[4-[2-(2,6-dioxo-3-piperidyl)-1-oxo- | 868.41 | 868 |
| | isoindolin-5-yl]piperazin-1-yl]-3-oxo- | | |
| | propoxy]ethoxy]ethoxy]ethylamino]-5-methyl-3-pyridyl]-3-(7- | | |
| | tetrahydrofuran-2-ylpyrazolo[1,5-a]pyrimidin-6-yl)urea | | |
| 30 | N-[2-(2,6-dioxo-3-piperidyl)-1-oxo-isoindolin-4-yl]-3-[2-[2-[2- | 799.35 | 799 |
| | [[3-methyl-5-[(7-tetrahydrofuran-2-ylpyrazolo[1,5-a]pyrimidin- | | |
| | 6-yl)carbamoylamino]-2- | | |
| | pyridyl]amino]ethoxy]ethoxy]ethoxy]propanamide | | |
| 31 | 1-[6-[4-[3-[2-[2-[4-[2-(2,6-dioxo-3-piperidyl)-1-oxo- | 893.44 | 893 |
| | isoindolin-5-yl]piperazin-1- | | |
| | yl]ethoxy]ethoxy]propanoyl]piperazin-1-yl]-5-methyl-3- | | |
| | pyridyl]-3-(7-tetrahydrofuran-2-ylpyrazolo[1,5-a]pyrimidin-6- | | |
| | yl)urea | | |
| 32 | N-[2-[[5-chloro-3-[(7-tetrahydrofuran-2-ylpyrazolo[1,5- | 888.36 | 888 |
| | a]pyrimidin-6-yl)carbamoylamino]-2-pyridyl]oxy]ethyl]-3-[2- | | |
| | [2-[4-[2-(2,6-dioxo-3-piperidyl)-1-oxo-isoindolin-5- | | |
| | yl]piperazin-1-yl]ethoxy]ethoxy]propanamide | | |
| 33 | N-[2-[2-[3-[4-[2-(2,6-dioxo-3-piperidyl)-1-oxo-isoindolin-5- | 918.40 | 918 |
| | yl]piperazin-1-yl]-3-oxo-propoxy]ethoxy]ethyl]-1-[3-methyl-5- | | |
| | [(7-tetrahydrofuran-2-ylpyrazolo[1,5-a]pyrimidin-6- | | |
| | yl)carbamoylamino]-2-pyridyl]pyrazole-4-carboxamide | | |
| 34 | N-[2-[2-[3-[4-[2-(2,6-dioxo-3-piperidyl)-1-oxo-isoindolin-5- | 918.40 | 918 |
| | yl]piperazin-1-yl]-3-oxo-propoxy]ethoxy]ethyl]-1-[3-methyl-5- | | |
| | [(7-tetrahydrofuran-2-ylpyrazolo[1,5-a]pyrimidin-6- | | |
| | yl)carbamoylamino]-2-pyridyl]imidazole-4-carboxamide | | |
| 35 | 1-[6-[4-[3-[2-[4-[2-(2,6-dioxo-3-piperidyl)-1-oxo-isoindolin-5- | 849.42 | 849 |
| | yl]piperazin-1-yl]ethoxy]propanoyl]piperazin-1-yl]-5-methyl- | | |
| | 3-pyridyl]-3-(7-tetrahydrofuran-2-ylpyrazolo[1,5-a]pyrimidin- | | |
| | 6-yl)urea | | |
| 36 | N-[2-[5-chloro-3-[(7-tetrahydrofuran-2-ylpyrazolo[1,5- | 844.33 | 844 |
| | a]pyrimidin-6-yl)carbamoylamino]-2-pyridyl]oxy]ethyl]-3-[2- | | |
| | [4-[2-(2,6-dioxo-3-piperidyl)-1-oxo-isoindolin-5-yl]piperazin- | | |
| | 1-yl]ethoxy]propanamide | | |
| 37 | N-[2-[5-chloro-3-[(7-cyclopentylpyrazolo[1,5-a]pyrimidin-6- | 842.35 | 842 |
| | yl)carbamoylamino]-2-pyridyl]oxy]ethyl]-3-[2-[4-[2-(2,6- | | |
| | dioxo-3-piperidyl)-1-oxo-isoindolin-5-yl]piperazin-1- | | |
| | yl]ethoxy]propanamide | | |
| 38 | N-[2-[[5-chloro-3-[(7-cyclopentylpyrazolo[1,5-a]pyrimidin-6- | 886.38 | 886 |
| | yl)carbamoylamino]-2-pyridyl]oxy]ethyl]-3-[2-[2-[4-[2-(2,6- | | |
| | dioxo-3-piperidyl)-1-oxo-isoindolin-5-yl]piperazin-1- | | |
| | yl]ethoxy]ethoxy]propanamide | | |
| 39 | N-[2-[[5-chloro-3-[(7-tetrahydrofuran-3-ylpyrazolo[1,5- | 844.33 | 844 |
| | a]pyrimidin-6-yl)carbamoylamino]-2-pyridyl]oxy]ethyl]-3-[2- | | |
| | [4-[2-(2,6-dioxo-3-piperidyl)-1-oxo-isoindolin-5-yl]piperazin- | | |
| | 1-yl]ethoxy]propanamide | | |
| 40 | N-[2-[[5-chloro-3-[(7-tetrahydrofuran-3-ylpyrazolo[1,5- | 888.36 | 888 |
| | a]pyrimidin-6-yl)carbamoylamino]-2-pyridyl]oxy]ethyl]-3-[2- | | |
| | [2-[4-[2-(2,6-dioxo-3-piperidyl)-1-oxo-isoindolin-5- | | |
| | yl]piperazin-1-yl]ethoxy]ethoxy]propanamide | | |
| 41 | N-[2-[5-chloro-3-[(7-tetrahydropyran-4-ylpyrazolo[1,5- | 902.37 | 902 |
| | a]pyrimidin-6-yl)carbamoylamino]-2-pyridyl]oxy]ethyl]-3-[2- | | |
| | [2-[4-[2-(2,6-dioxo-3-piperidyl)-1-oxo-isoindolin-5- | | |
| | yl]piperazin-1-yl]ethoxy]ethoxy]propanamide | | |
| 42 | N-[2-[[5-chloro-3-[(7-tetrahydropyran-4-ylpyrazolo[1,5- | 858.35 | 858 |
| | a]pyrimidin-6-yl)carbamoylamino]-2-pyridyl]oxy]ethyl]-3-[2- | | |
| | [4-[2-(2,6-dioxo-3-piperidyl)-1-oxo-isoindolin-5-yl]piperazin- | | |
| | 1-yl]ethoxy]propanamide | | |
| 43 | N-[2-[4-chloro-2-[(7-cyclopentylpyrazolo[1,5-a]pyrimidin-6- | 841.36 | 841 |
| | yl)carbamoylamino]phenoxy]ethyl]-3-[2-[4-[2-(2,6-dioxo-3- | | |
| | piperidyl)-1-oxo-isoindolin-5-yl]piperazin-1- | | |
| | yl]ethoxy]propanamide | | |
| 44 | N-[2-[[5-chloro-3-[(7-cyclohexylpyrazolo[1,5-a]pyrimidin-6- | 856.3 | 856 |
| | yl)carbamoylamino]-2-pyridyl]oxy]ethyl]-3-[2-[4-[2-(2,6- | | |
| | dioxo-3-piperidyl)-1-oxo-isoindolin-5-yl]piperazin-1- | | |
| | yl]ethoxy]propanamide | | |
| 45 | N1-(7-cyclopentylpyrazolo[1,5-a]pyrimidin-6-yl)-N2-[2-[3-[2- | 812.42 | 812 |
| | [4-[2-(2,6-dioxo-3-piperidyl)-1-oxo-isoindolin-5-yl]piperazin- | | |
| | 1-yl]ethoxy]propanoylamino]ethyl]pyrrolidine-1,2- | | |
| | dicarboxamide | | |
| 46 | (2S,4R)-N1-(7-cyclopentylpyrazolo[1,5-a]pyrimidin-6-yl)-N2- | 828.42 | 828 |
| | [2-[3-[2-[4-[2-(2,6-dioxo-3-piperidyl)-1-oxo-isoindolin-5- | | |
| | yl]piperazin-1-yl]ethoxy]propanoylamino]ethyl]-4-hydroxy- | | |
| | pyrrolidine-1,2-dicarboxamide | | |
| 47 | (2S,4R)-N1-(7-cyclopentylpyrazolo[1,5-a]pyrimidin-6-yl)-N2- | 872.44 | 872 |
| | [2-[3-[2-[2-[4-[2-(2,6-dioxo-3-piperidyl)-1-oxo-isoindolin-5- | | |
| | yl]piperazin-1-yl]ethoxy]ethoxy]propanoylamino]ethyl]-4- | | |
| | hydroxy-pyrrolidine-1,2-dicarboxamide | | |
| 48 | N1-(7-cyclopentylpyrazolo[1,5-a]pyrimidin-6-yl)-N2-[2-[3-[2- | 856.45 | 856 |
| | [2-[4-[2-(2,6-dioxo-3-piperidyl)-1-oxo-isoindolin-5- | | |
| | yl]piperazin-1- | | |
| | yl]ethoxy]ethoxy]propanoylamino]ethyl]pyrrolidine-1,2- | | |
| | dicarboxamide | | |
| 49 | N-[2-[4-chloro-2-[(7-cyclopentylpyrazolo[1,5-a]pyrimidin-6- | 885.38 | 885 |
| | yl)carbamoylamino]phenoxy]ethyl]-3-[2-[2-[4-[2-(2,6-dioxo-3- | | |
| | piperidyl)-1-oxo-isoindolin-5-yl]piperazin-1- | | |
| | yl]ethoxy]ethoxy]propanamide | | |
| 50 | N-[2-[[5-chloro-3-[(7-cyclohexylimidazo[1,2-a]pyrimidin-6- | 856.37 | 856 |
| | yl)carbamoylamino]-2-pyridyl]oxy]ethyl]-3-[2-[4-[2-(2,6- | | |
| | dioxo-3-piperidyl)-1-oxo-isoindolin-5-yl]piperazin-1- | | |
| | yl]ethoxy]propanamide | | |
| 51 | N-[2-[[5-chloro-3-[(7-cyclohexyl-[1,2,4]triazolo[1,5- | 857.36 | 857 |
| | a]pyrimidin-6-yl)carbamoylamino]-2-pyridyl]oxy]ethyl]-3-[2- | | |
| | [4-[2-(2,6-dioxo-3-piperidyl)-1-oxo-isoindolin-5-yl]piperazin- | | |
| | 1-yl]ethoxy]propanamide | | |
| 52 | N-[2-[[5-chloro-3-[(5-cyclopentylimidazo[1,2-a]pyrimidin-6- | 842.35 | 842 |
| | yl)carbamoylamino]-2-pyridyl]oxy]ethyl]-3-[2-[4-[2-(2,6- | | |
| | dioxo-3-piperidyl)-1-oxo-isoindolin-5-yl]piperazin-1- | | |
| | yl]ethoxy]propanamide | | |
| 53 | N-[2-[[5-chloro-3-[(5-cyclopentylimidazo[1,2-a]pyrimidin-6- | 886.38 | 886 |
| | yl)carbamoylamino]-2-pyridyl]oxy]ethyl]-3-[2-[2-[4-[2-(2,6- | | |
| | dioxo-3-piperidyl)-1-oxo-isoindolin-5-yl]piperazin-1- | | |
| | yl]ethoxy]ethoxy]propanamide | | |
| 54 | 1-(7-cyclopentylpyrazolo[1,5-a]pyrimidin-6-yl)-3-[6-[4-[2- | 663.32 | 663 |
| | (2,6-dioxo-3-piperidyl)-1-oxo-isoindolin-5-yl]piperazin-1-yl]- | | |
| | 5-methyl-3-pyridyl]urea | | |
| 55 | 1-(7-cyclopentylpyrazolo[1,5-a]pyrimidin-6-yl)-3-[6-[5-[5-[4- | 833.36 | 833 |
| | [2-(2,6-dioxo-3-piperidyl)-6-fluoro-1,3-dioxo-isoindolin-5- | | |
| | yl]piperazin-1-yl]pentyl]-1,2,4-oxadiazol-3-yl]-5-methyl-3- | | |
| | pyridyl]urea | | |
| 56 | 1-[5-chloro-6-[5-[[4-[2-(2,6-dioxo-3-piperidyl)-1-oxo- | 765.28 | 765 |
| | isoindolin-5-yl]piperazin-1-yl]methyl]-1,2,4-oxadiazol-3-yl]-3- | | |
| | pyridyl]-3-(7-cyclopentylpyrazolo[1,5-a]pyrimidin-6-yl)urea | | |
| 57 | 1-(7-cyclopentylpyrazolo[1,5-a]pyrimidin-6-yl)-3-[6-[5-[4-[4- | 819.35 | 819 |
| | [2-(2,6-dioxo-3-piperidyl)-6-fluoro-1,3-dioxo-isoindolin-5- | | |
| | yl]piperazin-1-yl]butyl]-1,2,4-oxadiazol-3-yl]-5-methyl-3- | | |
| | pyridyl]urea | | |
| 58 | 1-[6-[5-[4-[4-[2-(2,6-dioxo-3-piperidyl)-1-oxo-isoindolin-5- | 803.34 | 803 |
| | yl]piperazin-1-yl]-4-oxo-butyl]-1,2,4-oxadiazol-3-yl]-5-methyl- | | |
| | 3-pyridyl]-3-(7-tetrahydrofuran-2-ylpyrazolo[1,5-a]pyrimidin- | | |
| | 6-yl)urea | | |
| 59 | 1-(7-cyclopentyl-2-methyl-pyrazolo[1,5-a]pyrimidin-6-yl)-3- | 829.39 | 829 |
| | [6-[5-[5-[4-[2-(2,6-dioxo-3-piperidyl)-1-oxo-isoindolin-5- | | |
| | yl]piperazin-1-yl]-5-oxo-pentyl]-1,2,4-oxadiazol-3-yl]-5- | | |
| | methyl-3-pyridyl]urea | | |
| 60 | 1-[6-[5-[3-[4-[2-(2,6-dioxo-3-piperidyl)-1-oxo-isoindolin-5- | 789.32 | 789 |
| | yl]piperazin-1-yl]-3-oxo-propyl]-1,2,4-oxadiazol-3-yl]-5- | | |
| | methyl-3-pyridyl]-3-(7-tetrahydrofuran-2-ylpyrazolo[1,5- | | |
| | a]pyrimidin-6-yl)urea | | |
| 61 | 6-[3-[5-[(7-cyclopentylpyrazolo[1,5-a]pyrimidin-6- | 760.33 | 760 |
| | yl)carbamoylamino]-3-methyl-2-pyridyl]-1,2,4-oxadiazol-5- | | |
| | yl]-N-[2-(2,6-dioxo-3-piperidyl)-1-oxo-isoindolin-5- | | |
| | yl]hexanamide | | |
| 62 | 1-[6-[5-[3-[4-[2-(2,6-dioxo-3-piperidyl)-1-oxo-isoindolin-5- | 775.34 | 775 |
| | yl]piperazin-1-yl]propyl]-1,2,4-oxadiazol-3-yl]-5-methyl-3- | | |
| | pyridyl]-3-(7-tetrahydrofuran-2-ylpyrazolo[1,5-a]pyrimidin-6- | | |
| | yl)urea | | |
| 63 | 1-[6-[5-[4-[4-[2-(2,6-dioxo-3-piperidyl)-1-oxo-isoindolin-5- | 789.36 | 789 |
| | yl]piperazin-1-yl]butyl]-1,2,4-oxadiazol-3-yl]-5-methyl-3- | | |
| | pyridyl]-3-(7-tetrahydrofuran-2-ylpyrazolo[1,5-a]pyrimidin-6- | | |
| | yl)urea | | |
| 64 | 1-(7-cyclopentylpyrazolo[1,5-a]pyrimidin-6-yl)-3-[5-methyl-6- | 815.41 | 815 |
| | [5-[5-[4-[2-(1-methyl-2,6-dioxo-3-piperidyl)-1-oxo-isoindolin- | | |
| | 5-yl]piperazin-1-yl]pentyl]-1,2,4-oxadiazol-3-yl]-3- | | |
| | pyridyl]urea | | |
| 65 | 1-[5-chloro-6-[5-[6-[4-[2-(2,6-dioxo-3-piperidyl)-1-oxo- | 795.29 | 795 |
| | isoindolin-5-yl]piperazin-1-yl]-6-oxo-hexyl]-1,2,4-oxadiazol-3- | | |
| | yl]-3-pyridyl]-3-(7-methylpyrazolo[1,5-a]pyrimidin-6-yl)urea | | |
| 66 | 1-[5-chloro-6-[5-[6-[4-[2-(2,6-dioxo-3-piperidyl)-1-oxo- | 823.32 | 823 |
| | isoindolin-5-yl]piperazin-1-yl]-6-oxo-hexyl]-1,2,4-oxadiazol-3- | | |
| | yl]-3-pyridyl]-3-(7-isopropylpyrazolo[1,5-a]pyrimidin-6- | | |
| | yl)urea | | |
| 67 | 1-[6-[5-[6-[4-[2-(2,6-dioxo-3-piperidyl)-1-oxo-isoindolin-5- | 845.38 | 845 |
| | yl]piperazin-1-yl]-6-oxo-hexyl]-1,2,4-oxadiazol-3-yl]-5- | | |
| | methyl-3-pyridyl]-3-(2-methyl-7-tetrahydrofuran-2-yl- | | |
| | pyrazolo[1,5-a]pyrimidin-6-yl)urea | | |
| 68 | 1-(7-cyclopentylpyrazolo[1,5-a]pyrimidin-6-yl)-3-[6-[5-[6-[4- | 815.41 | 815 |
| | [2-(2,6-dioxo-3-piperidyl)-1-oxo-isoindolin-5-yl]piperazin-1- | | |
| | yl]hexyl]-1,2,4-oxadiazol-3-yl]-5-methyl-3-pyridyl]urea | | |
| 69 | 1-(7-cyclopentyl-2-methyl-pyrazolo[1,5-a]pyrimidin-6-yl)-3- | 815.41 | 815 |
| | [6-[5-[5-[4-[2-(2,6-dioxo-3-piperidyl)-1-oxo-isoindolin-5- | | |
| | yl]piperazin-1-yl]pentyl]-1,2,4-oxadiazol-3-yl]-5-methyl-3- | | |
| | pyridyl]urea | | |
| 70 | [3-[5-[4-[6-[3-[3-chloro-5-[(7-cyclopentylpyrazolo[1,5- | 963.40 | 963 |
| | a]pyrimidin-6-yl)carbamoylamino]-2-pyridyl]-1,2,4-oxadiazol- | | |
| | 5-yl]hexanoyl]piperazin-1-yl]-1-oxo-isoindolin-2-yl]-2,6- | | |
| | dioxo-1-piperidyl]methyl 2,2-dimethylpropanoate | | |
| 71 | 1-[5-chloro-6-[5-[4-[4-[2-(2,6-dioxo-3-piperidyl)-1-oxo- | 821.30 | 821 |
| | isoindolin-5-yl]piperazin-1-yl]-4-oxo-butyl]-1,2,4-oxadiazol-3- | | |
| | yl]-3-pyridyl]-3-(7-cyclopentylpyrazolo[1,5-a]pyrimidin-6- | | |
| | yl)urea | | |
| 72 | 1-[6-[5-[6-[4-[2-(2,6-dioxo-3-piperidyl)-1-oxo-isoindolin-5- | 817.39 | 817 |
| | yl]piperazin-1-yl]hexyl]-1,2,4-oxadiazol-3-yl]-5-methyl-3- | | |
| | pyridyl]-3-(7-tetrahydrofuran-2-ylpyrazolo[1,5-a]pyrimidin-6- | | |
| | yl)urea | | |
| 73 | 1-[6-[5-[6-[4-[2-(2,6-dioxo-3-piperidyl)-1-oxo-isoindolin-5- | 831.41 | 831 |
| | yl]piperazin-1-yl]hexyl]-1,2,4-oxadiazol-3-yl]-5-methyl-3- | | |
| | pyridyl]-3-(2-methyl-7-tetrahydrofuran-2-yl-pyrazolo[1,5- | | |
| | a]pyrimidin-6-yl)urea | | |
| 74 | 1-(7-cyclopentylpyrazolo[1,5-a]pyrimidin-6-yl)-3-[6-[5-[6-[4- | 833.36 | 833 |
| | [2-(2,6-dioxo-3-piperidyl)-1-oxo-isoindolin-5-yl]piperazin-1- | | |
| | yl]-6-oxo-hexyl]-1,2,4-oxadiazol-3-yl]-5-fluoro-3-pyridyl]urea | | |
| 75 | 1-[5-chloro-6-[5-[4-[4-[2-(2,6-dioxo-3-piperidyl)-1-oxo- | 807.32 | 807 |
| | isoindolin-5-yl]piperazin-1-yl]butyl]-1,2,4-oxadiazol-3-yl]-3- | | |
| | pyridyl]-3-(7-cyclopentylpyrazolo[1,5-a]pyrimidin-6-yl)urea | | |
| 76 | 1-[5-chloro-6-[5-[5-[4-[2-(2,6-dioxo-3-piperidyl)-1-oxo- | 835.32 | 835 |
| | isoindolin-5-yl]piperazin-1-yl]-5-oxo-pentyl]-1,2,4-oxadiazol- | | |
| | 3-yl]-3-pyridyl]-3-(7-cyclopentylpyrazolo[1,5-a]pyrimidin-6- | | |
| | yl)urea | | |
| 77 | 1-(7-cyclopentylpyrazolo[1,5-a]pyrimidin-6-yl)-3-[6-[5-[4-[4- | 805.33 | 805 |
| | [2-(2,6-dioxo-3-piperidyl)-1-oxo-isoindolin-5-yl]piperazin-1- | | |
| | yl]-4-oxo-butyl]-1,2,4-oxadiazol-3-yl]-5-fluoro-3-pyridyl]urea | | |
| 78 | 1-[5-chloro-6-[5-[6-[4-[2-(2,6-dioxo-3-piperidyl)-1-oxo- | 835.36 | 835 |
| | isoindolin-5-yl]piperazin-1-yl]hexyl]-1,2,4-oxadiazol-3-yl]-3- | | |
| | pyridyl]-3-(7-cyclopentylpyrazolo[1,5-a]pyrimidin-6-yl)urea | | |
| 79 | 1-[5-chloro-6-[5-[5-[4-[2-(2,6-dioxo-3-piperidyl)-1-oxo- | 821.34 | 821 |
| | isoindolin-5-yl]piperazin-1-yl]pentyl]-1,2,4-oxadiazol-3-yl]-3- | | |
| | pyridyl]-3-(7-cyclopentylpyrazolo[1,5-a]pyrimidin-6-yl)urea | | |
| 80 | 4-[3-[3-chloro-5-[(7-cyclopentylpyrazolo[1,5-a]pyrimidin-6- | 752.25 | 752 |
| | yl)carbamoylamino]-2-pyridyl]-1,2,4-oxadiazol-5-yl]-N-[2- | | |
| | (2,6-dioxo-3-piperidyl)-1-oxo-isoindolin-5-yl]butanamide | | |
| 81 | 1-(7-cyclopentylpyrazolo[1,5-a]pyrimidin-6-yl)-3-[6-[5-[5-[4- | 819.35 | 819 |
| | [2-(2,6-dioxo-3-piperidyl)-1-oxo-isoindolin-5-yl]piperazin-1- | | |
| | yl]-5-oxo-pentyl]-1,2,4-oxadiazol-3-yl]-5-fluoro-3-pyridyl]urea | | |
| 82 | 1-(7-cyclopentyl-2-methyl-pyrazolo[1,5-a]pyrimidin-6-yl)-3- | 843.41 | 843 |
| | [6-[5-[6-[4-[2-(2,6-dioxo-3-piperidyl)-1-oxo-isoindolin-5- | | |
| | yl]piperazin-1-yl]-6-oxo-hexyl]-1,2,4-oxadiazol-3-yl]-5- | | |
| | methyl-3-pyridyl]urea | | |
| 83 | 1-(7-cyclopentyl-2-methyl-pyrazolo[1,5-a]pyrimidin-6-yl)-3- | 829.43 | 829 |
| | [6-[5-[6-[4-[2-(2,6-dioxo-3-piperidyl)-1-oxo-isoindolin-5- | | |
| | yl]piperazin-1-yl]hexyl]-1,2,4-oxadiazol-3-yl]-5-methyl-3- | | |
| | pyridyl]urea | | |
| 84 | 1-[6-[5-[5-[4-[2-(2,6-dioxo-3-piperidyl)-1-oxo-isoindolin-5- | 831.37 | 831 |
| | yl]piperazin-1-yl]-5-oxo-pentyl]-1,2,4-oxadiazol-3-yl]-5- | | |
| | methyl-3-pyridyl]-3-(2-methyl-7-tetrahydrofuran-2-yl- | | |
| | pyrazolo[1,5-a]pyrimidin-6-yl)urea | | |
| 85 | 1-[6-[5-[5-[4-[2-(2,6-dioxo-3-piperidyl)-1-oxo-isoindolin-5- | 817.39 | 817 |
| | yl]piperazin-1-yl]pentyl]-1,2,4-oxadiazol-3-yl]-5-methyl-3- | | |
| | pyridyl]-3-(2-methyl-7-tetrahydrofuran-2-yl-pyrazolo[1,5- | | |
| | a]pyrimidin-6-yl)urea | | |
| 86 | 1-[6-[5-[5-[4-[2-(2,6-dioxo-3-piperidyl)-1-oxo-isoindolin-5- | 789.36 | 789 |
| | yl]piperazin-1-yl]-5-oxo-pentyl]-1,2,4-oxadiazol-3-yl]-5- | | |
| | methyl-3-pyridyl]-3-(7-isopropylpyrazolo[1,5-a]pyrimidin-6- | | |
| | yl)urea | | |
| 87 | 1-[6-[5-[5-[4-[2-(2,6-dioxo-3-piperidyl)-1-oxo-isoindolin-5- | 775.38 | 775 |
| | yl]piperazin-1-yl]pentyl]-1,2,4-oxadiazol-3-yl]-5-methyl-3- | | |
| | pyridyl]-3-(7-isopropylpyrazolo[1,5-a]pyrimidin-6-yl)urea | | |
| 88 | [3-[5-[4-[5-[3-[5-[(7-cyclopentylpyrazolo[1,5-a]pyrimidin-6- | 915.46 | 915 |
| | yl)carbamoylamino]-3-methyl-2-pyridyl]-1,2,4-oxadiazol-5- | | |
| | yl]pentyl]piperazin-1-yl]-1-oxo-isoindolin-2-yl]-2,6-dioxo-1- | | |
| | piperidyl]methyl 2,2-dimethylpropanoate | | |
| 89 | 1-[6-[5-[4-[4-[2-(2,6-dioxo-3-piperidyl)-1-oxo-isoindolin-5- | 775.34 | 775 |
| | yl]piperazin-1-yl]-4-oxo-butyl]-1,2,4-oxadiazol-3-yl]-5-methyl- | | |
| | 3-pyridyl]-3-(7-isopropylpyrazolo[1,5-a]pyrimidin-6-yl)urea | | |
| 90 | 1-[6-[5-[4-[4-[2-(2,6-dioxo-3-piperidyl)-1-oxo-isoindolin-5- | 761.36 | 761 |
| | yl]piperazin-1-yl]butyl]-1,2,4-oxadiazol-3-yl]-5-methyl-3- | | |
| | pyridyl]-3-(7-isopropylpyrazolo[1,5-a]pyrimidin-6-yl)urea | | |
| 91 | 1-[6-[5-[6-[4-[2-(2,6-dioxo-3-piperidyl)-1-oxo-isoindolin-5- | 803.37 | 803 |
| | yl]piperazin-1-yl]-6-oxo-hexyl]-1,2,4-oxadiazol-3-yl]-5- | | |
| | methyl-3-pyridyl]-3-(7-isopropylpyrazolo[1,5-a]pyrimidin-6- | | |
| | yl)urea | | |
| 92 | 1-[6-[5-[6-[4-[2-(2,6-dioxo-3-piperidyl)-1-oxo-isoindolin-5- | 789.39 | 789 |
| | yl]piperazin-1-yl]hexyl]-1,2,4-oxadiazol-3-yl]-5-methyl-3- | | |
| | pyridyl]-3-(7-isopropylpyrazolo[1,5-a]pyrimidin-6-yl)urea | | |
| 93 | 1-[5-chloro-6-[5-[6-[4-[2-(2,6-dioxo-3-piperidyl)-1-oxo- | 809.34 | 809 |
| | isoindolin-5-yl]piperazin-1-yl]hexyl]-1,2,4-oxadiazol-3-yl]-3- | | |
| | pyridyl]-3-(7-isopropylpyrazolo[1,5-a]pyrimidin-6-yl)urea | | |
| 94 | 1-(7-cyclopentylpyrazolo[1,5-a]pyrimidin-6-yl)-3-[6-[5-[5-[4- | 815.37 | 815 |
| | [2-(2,6-dioxo-3-piperidyl)-1-oxo-isoindolin-5-yl]piperazin-1- | | |
| | yl]-5-oxo-pentyl]-1,2,4-oxadiazol-3-yl]-4-methyl-3- | | |
| | pyridyl]urea | | |
| 95 | 1-(7-cyclopentylpyrazolo[1,5-a]pyrimidin-6-yl)-3-[6-[5-[6-[4- | 819.39 | 819 |
| | [2-(2,6-dioxo-3-piperidyl)-1-oxo-isoindolin-5-yl]piperazin-1- | | |
| | yl]hexyl]-1,2,4-oxadiazol-3-yl]-5-fluoro-3-pyridyl]urea | | |
| 96 | 1-(7-cyclopentylpyrazolo[1,5-a]pyrimidin-6-yl)-3-[6-[5-[5-[4- | 869.35 | 869 |
| | [2-(2,6-dioxo-3-piperidyl)-1-oxo-isoindolin-5-yl]piperazin-1- | | |
| | yl]-5-oxo-pentyl]-1,2,4-oxadiazol-3-yl]-5-(trifluoromethyl)-3- | | |
| | pyridyl]urea | | |
| 97 | 1-(7-cyclopentylpyrazolo[1,5-a]pyrimidin-6-yl)-3-[6-[5-[5-[4- | 805.37 | 805 |
| | [2-(2,6-dioxo-3-piperidyl)-1-oxo-isoindolin-5-yl]piperazin-1- | | |
| | yl]pentyl]-1,2,4-oxadiazol-3-yl]-5-fluoro-3-pyridyl]urea | | |
| 98 | 1-[5-chloro-6-[5-[5-[4-[2-(2,6-dioxo-3-piperidyl)-1-oxo- | 849.34 | 849 |
| | isoindolin-5-yl]piperazin-1-yl]-5-oxo-pentyl]-1,2,4-oxadiazol- | | |
| | 3-yl]-3-pyridyl]-3-(7-cyclohexylpyrazolo[1,5-a]pyrimidin-6- | | |
| | yl)urea | | |
| 99 | 1-[5-chloro-6-[5-[5-[4-[2-(2,6-dioxo-3-piperidyl)-1-oxo- | 835.36 | 835 |
| | isoindolin-5-yl]piperazin-1-yl]pentyl]-1,2,4-oxadiazol-3-yl]-3- | | |
| | pyridyl]-3-(7-cyclohexylpyrazolo[1,5-a]pyrimidin-6-yl)urea | | |
| 100 | 5-[3-[5-[(7-cyclopentylpyrazolo[1,5-a]pyrimidin-6- | 691.31 | 691 |
| | yl)carbamoylamino]-3-methyl-2-pyridyl]-1,2,4-oxadiazol-5- | | |
| | yl]-N-[3-(2,6-dioxo-3-piperidyl)phenyl]pentanamide | | |
| 101 | 5-[3-[5-[(7-cyclopentylpyrazolo[1,5-a]pyrimidin-6- | 691.31 | 691 |
| | yl)carbamoylamino]-3-methyl-2-pyridyl]-1,2,4-oxadiazol-5- | | |
| | yl]-N-[4-(2,6-dioxo-3-piperidyl)phenyl]pentanamide | | |
| 102 | [3-[5-[4-[6-[3-[5-[(7-cyclopentylpyrazolo[1,5-a]pyrimidin-6- | 943.46 | 943 |
| | yl)carbamoylamino]-3-methyl-2-pyridyl]-1,2,4-oxadiazol-5- | | |
| | yl]hexanoyl]piperazin-1-yl]-1-oxo-isoindolin-2-yl]-2,6-dioxo- | | |
| | 1-piperidyl]methyl 2,2-dimethylpropanoate | | |
| 103 | dibutyl [3-[5-[4-[6-[3-[3-chloro-5-[(7-cyclopentylpyrazolo[1,5- | 1071.43 | 1071 |
| | a]pyrimidin-6-yl)carbamoylamino]-2-pyridyl]-1,2,4-oxadiazol- | | |
| | 5-yl]hexanoyl]piperazin-1-yl]-1-oxo-isoindolin-2-yl]-2,6- | | |
| | dioxo-1-piperidyl]methyl phosphate | | |
| 104 | 1-(7-cyclopentylpyrazolo[1,5-a]pyrimidin-6-yl)-3-[6-[5-[6-[4- | 883.36 | 883 |
| | [2-(2,6-dioxo-3-piperidyl)-1-oxo-isoindolin-5-yl]piperazin-1- | | |
| | yl]-6-oxo-hexyl]-1,2,4-oxadiazol-3-yl]-5-(trifluoromethyl)-3- | | |
| | pyridyl]urea | | |
| 105 | 1-(7-cyclopentylpyrazolo[1,5-a]pyrimidin-6-yl)-3-[6-[5-[4-[4- | 791.35 | 791 |
| | [2-(2,6-dioxo-3-piperidyl)-1-oxo-isoindolin-5-yl]piperazin-1- | | |
| | yl]butyl]-1,2,4-oxadiazol-3-yl]-5-fluoro-3-pyridyl]urea | | |
| 106 | 1-(7-cyclopentylpyrazolo[1,5-a]pyrimidin-6-yl)-3-[6-[5-[4-[4- | 855.33 | 855 |
| | [2-(2,6-dioxo-3-piperidyl)-1-oxo-isoindolin-5-yl]piperazin-1- | | |
| | yl]-4-oxo-butyl]-1,2,4-oxadiazol-3-yl]-5-(trifluoromethyl)-3- | | |
| | pyridyl]urea | | |
| 107 | 1-(7-cyclohexylpyrazolo[1,5-a]pyrimidin-6-yl)-3-[6-[5-[6-[4- | 843.41 | 843 |
| | [2-(2,6-dioxo-3-piperidyl)-1-oxo-isoindolin-5-yl]piperazin-1- | | |
| | yl]-6-oxo-hexyl]-1,2,4-oxadiazol-3-yl]-5-methyl-3-pyridyl]urea | | |
| 108 | 1-(7-cyclopentylpyrazolo[1,5-a]pyrimidin-6-yl)-3-[6-[5-[5-[4- | 855.37 | 855 |
| | [2-(2,6-dioxo-3-piperidyl)-1-oxo-isoindolin-5-yl]piperazin-1- | | |
| | yl]pentyl]-1,2,4-oxadiazol-3-yl]-5-(trifluoromethyl)-3- | | |
| | pyridyl]urea | | |
| 109 | 6-[3-[5-[(7-cyclohexylpyrazolo[1,5-a]pyrimidin-6- | 774.35 | 774 |
| | yl)carbamoylamino]-3-methyl-2-pyridyl]-1,2,4-oxadiazol-5- | | |
| | yl]-N-[2-(2,6-dioxo-3-piperidyl)-1-oxo-isoindolin-5- | | |
| | yl]hexanamide | | |
| 110 | 1-[6-[5-[6-[4-[2-(2,6-dioxo-3-piperidyl)-1-oxo-isoindolin-5- | 789.36 | 789 |
| | yl]piperazin-1-yl]-6-oxo-hexyl]-1,3,4-oxadiazol-2-yl]-3- | | |
| | pyridyl]-3-(7-isopropylpyrazolo[1,5-a]pyrimidin-6-yl)urea | | |
| 111 | 1-(7-cyclopentylpyrazolo[1,5-a]pyrimidin-6-yl)-3-[6-[5-[6-[4- | 815.37 | 815 |
| | [2-(2,6-dioxo-3-piperidyl)-1-oxo-isoindolin-5-yl]piperazin-1- | | |
| | yl]-6-oxo-hexyl]-1,3,4-oxadiazol-2-yl]-3-pyridyl]urea | | |
| 112 | 1-(7-cyclobutylpyrazolo[1,5-a]pyrimidin-6-yl)-3-[6-[5-[5-[4- | 801.36 | 801 |
| | [2-(2,6-dioxo-3-piperidyl)-1-oxo-isoindolin-5-yl]piperazin-1- | | |
| | yl]-5-oxo-pentyl]-1,2,4-oxadiazol-3-yl]-5-methyl-3- | | |
| | pyridyl]urea | | |
| 113 | 1-(7-cyclobutylpyrazolo[1,5-a]pyrimidin-6-yl)-3-[6-[5-[5-[4- | 787.38 | 787 |
| | [2-(2,6-dioxo-3-piperidyl)-1-oxo-isoindolin-5-yl]piperazin-1- | | |
| | yl]pentyl]-1,2,4-oxadiazol-3-yl]-5-methyl-3-pyridyl]urea | | |
| 114 | 1-(7-cyclobutylpyrazolo[1,5-a]pyrimidin-6-yl)-3-[6-[5-[6-[4- | 815.37 | 815 |
| | [2-(2,6-dioxo-3-piperidyl)-1-oxo-isoindolin-5-yl]piperazin-1- | | |
| | yl]-6-oxo-hexyl]-1,2,4-oxadiazol-3-yl]-5-methyl-3-pyridyl]urea | | |
| 115 | 1-(7-cyclobutylpyrazolo[1,5-a]pyrimidin-6-yl)-3-[6-[5-[6-[4- | 801.39 | 801 |
| | [2-(2,6-dioxo-3-piperidyl)-1-oxo-isoindolin-5-yl]piperazin-1- | | |
| | yl]hexyl]-1,2,4-oxadiazol-3-yl]-5-methyl-3-pyridyl]urea | | |
| 116 | 1-[5-chloro-6-[5-[6-[4-[2-(2,6-dioxo-3-piperidyl)-1-oxo- | 835.32 | 835 |
| | isoindolin-5-yl]piperazin-1-yl]-6-oxo-hexyl]-1,2,4-oxadiazol-3- | | |
| | yl]-3-pyridyl]-3-(7-cyclobutylpyrazolo[1,5-a]pyrimidin-6- | | |
| | yl)urea | | |
| 117 | 1-[5-chloro-6-[5-[6-[4-[2-(2,6-dioxo-3-piperidyl)-1-oxo- | 821.34 | 821 |
| | isoindolin-5-yl]piperazin-1-yl]hexyl]-1,2,4-oxadiazol-3-yl]-3- | | |
| | pyridyl]-3-(7-cyclobutylpyrazolo[1,5-a]pyrimidin-6-yl)urea | | |
| 118 | 1-[5-chloro-6-[5-[6-[4-[2-(2,6-dioxo-3-piperidyl)-1-oxo- | 863.35 | 863 |
| | isoindolin-5-yl]piperazin-1-yl]-6-oxo-hexyl]-1,2,4-oxadiazol-3- | | |
| | yl]-3-pyridyl]-3-(7-cyclohexylpyrazolo[1,5-a]pyrimidin-6- | | |
| | yl)urea | | |
| 119 | 1-[5-chloro-6-[5-[6-[4-[2-(2,6-dioxo-3-piperidyl)-1-oxo- | 849.37 | 849 |
| | isoindolin-5-yl]piperazin-1-yl]hexyl]-1,2,4-oxadiazol-3-yl]-3- | | |
| | pyridyl]-3-(7-cyclohexylpyrazolo[1,5-a]pyrimidin-6-yl)urea | | |
| 120 | 1-[5-chloro-6-[5-[4-[4-[2-(2,6-dioxo-3-piperidyl)-1-oxo- | 835.32 | 835 |
| | isoindolin-5-yl]piperazin-1-yl]-4-oxo-butyl]-1,2,4-oxadiazol-3- | | |
| | yl]-3-pyridyl]-3-(7-cyclohexylpyrazolo[1,5-a]pyrimidin-6- | | |
| | yl)urea | | |
| 121 | 1-[5-chloro-6-[5-[4-[4-[2-(2,6-dioxo-3-piperidyl)-1-oxo- | 821.34 | 821 |
| | isoindolin-5-yl]piperazin-1-yl]butyl]-1,2,4-oxadiazol-3-yl]-3- | | |
| | pyridyl]-3-(7-cyclohexylpyrazolo[1,5-a]pyrimidin-6-yl)urea | | |
| 122 | 1-(7-cyclohexylpyrazolo[1,5-a]pyrimidin-6-yl)-3-[6-[5-[6-[4- | 829.43 | 829 |
| | [2-(2,6-dioxo-3-piperidyl)-1-oxo-isoindolin-5-yl]piperazin-1- | | |
| | yl]hexyl]-1,2,4-oxadiazol-3-yl]-5-methyl-3-pyridyl]urea | | |
| 123 | 4-[3-[5-[(7-cyclohexylpyrazolo[1,5-a]pyrimidin-6- | 746.32 | 746 |
| | yl)carbamoylamino]-3-methyl-2-pyridyl]-1,2,4-oxadiazol-5- | | |
| | yl]-N-[2-(2,6-dioxo-3-piperidyl)-1-oxo-isoindolin-5- | | |
| | yl]butanamide | | |
| 124 | 1-(7-cyclohexylpyrazolo[1,5-a]pyrimidin-6-yl)-3-[6-[5-[5-[4- | 829.39 | 829 |
| | [2-(2,6-dioxo-3-piperidyl)-1-oxo-isoindolin-5-yl]piperazin-1- | | |
| | yl]-5-oxo-pentyl]-1,2,4-oxadiazol-3-yl]-5-methyl-3- | | |
| | pyridyl]urea | | |
| 125 | 5-[3-[5-[(7-cyclohexylpyrazolo[1,5-a]pyrimidin-6- | 760.33 | 760 |
| | yl)carbamoylamino]-3-methyl-2-pyridyl]-1,2,4-oxadiazol-5- | | |
| | yl]-N-[2-(2,6-dioxo-3-piperidyl)-1-oxo-isoindolin-5- | | |
| | yl]pentanamide | | |
| 126 | 1-(7-cyclohexylpyrazolo[1,5-a]pyrimidin-6-yl)-3-[6-[5-[4-[4- | 815.37 | 815 |
| | [2-(2,6-dioxo-3-piperidyl)-1-oxo-isoindolin-5-yl]piperazin-1- | | |
| | yl]-4-oxo-butyl]-1,2,4-oxadiazol-3-yl]-5-methyl-3-pyridyl]urea | | |
| 127 | 1-(7-tert-butylpyrazolo[1,5-a]pyrimidin-6-yl)-3-[6-[5-[5-[4-[2- | 803.37 | 803 |
| | (2,6-dioxo-3-piperidyl)-1-oxo-isoindolin-5-yl]piperazin-1-yl]- | | |
| | 5-oxo-pentyl]-1,2,4-oxadiazol-3-yl]-5-methyl-3-pyridyl]urea | | |
| 128 | 1-(7-cyclopentylpyrazolo[1,5-a]pyrimidin-6-yl)-3-[6-[5-[6-[4- | 831.35 | 831 |
| | [2-(2,6-dioxo-3-piperidyl)-1-oxo-isoindolin-5-yl]piperazin-1- | | |
| | yl]-6-oxo-hexyl]-1,3,4-thiadiazol-2-yl]-3-pyridyl]urea | | |
| 129 | 1-[6-[5-[6-[4-[2-(2,6-dioxo-3-piperidyl)-1-oxo-isoindolin-5- | 805.34 | 805 |
| | yl]piperazin-1-yl]-6-oxo-hexyl]-1,3,4-thiadiazol-2-yl]-3- | | |
| | pyridyl]-3-(7-isopropylpyrazolo[1,5-a]pyrimidin-6-yl)urea | | |
| 130 | [3-[5-[4-[5-[3-[5-[(7-isopropylpyrazolo[1,5-a]pyrimidin-6- | 903.43 | 903 |
| | yl)carbamoylamino]-3-methyl-2-pyridyl]-1,2,4-oxadiazol-5- | | |
| | yl]pentanoyl]piperazin-1-yl]-1-oxo-isoindolin-2-yl]-2,6-dioxo- | | |
| | 1-piperidyl]methyl 2,2-dimethylpropanoate | | |
| 131 | [3-[5-[4-[6-[3-[5-[(7-isopropylpyrazolo[1,5-a]pyrimidin-6- | 917.44 | 917 |
| | yl)carbamoylamino]-3-methyl-2-pyridyl]-1,2,4-oxadiazol-5- | | |
| | yl]hexanoyl]piperazin-1-yl]-1-oxo-isoindolin-2-yl]-2,6-dioxo- | | |
| | 1-piperidyl]methyl 2,2-dimethylpropanoate | | |
| 132 | 1-(7-cyclohexylpyrazolo[1,5-a]pyrimidin-6-yl)-3-[6-[5-[5-[4- | 815.41 | 815 |
| | [2-(2,6-dioxo-3-piperidyl)-1-oxo-isoindolin-5-yl]piperazin-1- | | |
| | yl]pentyl]-1,2,4-oxadiazol-3-yl]-5-methyl-3-pyridyl]urea | | |
| 133 | 1-(7-cyclopentylpyrazolo[1,5-a]pyrimidin-6-yl)-3-[6-[5-[6-[4- | 869.38 | 869 |
| | [2-(2,6-dioxo-3-piperidyl)-1-oxo-isoindolin-5-yl]piperazin-1- | | |
| | yl]hexyl]-1,2,4-oxadiazol-3-yl]-5-(trifluoromethyl)-3- | | |
| | pyridyl]urea | | |
| 134 | [3-[5-[4-[5-[3-[3-chloro-5-[(7-cyclopentylpyrazolo[1,5- | 949.39 | 949 |
| | a]pyrimidin-6-yl)carbamoylamino]-2-pyridyl]-1,2,4-oxadiazol- | | |
| | 5-yl]pentanoyl]piperazin-1-yl]-1-oxo-isoindolin-2-yl]-2,6- | | |
| | dioxo-1-piperidyl]methyl 2,2-dimethylpropanoate | | |
| 135 | 1-[5-chloro-6-[5-[4-[4-[2-(1-methyl-2,6-dioxo-3-piperidyl)-1- | 809.30 | 809 |
| | oxo-isoindolin-5-yl]piperazin-1-yl]-4-oxo-butyl]-1,2,4- | | |
| | oxadiazol-3-yl]-3-pyridyl]-3-(7-isopropylpyrazolo[1,5- | | |
| | a]pyrimidin-6-yl)urea | | |
| 136 | 1-[5-chloro-6-[5-[5-[4-[2-(1-methyl-2,6-dioxo-3-piperidyl)-1- | 823.32 | 823 |
| | oxo-isoindolin-5-yl]piperazin-1-yl]-5-oxo-pentyl]-1,2,4- | | |
| | oxadiazol-3-yl]-3-pyridyl]-3-(7-isopropylpyrazolo[1,5- | | |
| | a]pyrimidin-6-yl)urea | | |
| 137 | [3-[5-[4-[4-[3-[3-chloro-5-[(7-isopropylpyrazolo[1,5- | 909.36 | 909 |
| | a]pyrimidin-6-yl)carbamoylamino]-2-pyridyl]-1,2,4-oxadiazol- | | |
| | 5-yl]butanoyl]piperazin-1-yl]-1-oxo-isoindolin-2-yl]-2,6-dioxo- | | |
| | 1-piperidyl]methyl 2,2-dimethylpropanoate | | |
| 138 | [3-[5-[4-[5-[3-[3-chloro-5-[(7-isopropylpyrazolo[1,5- | 923.37 | 923 |
| | a]pyrimidin-6-yl)carbamoylamino]-2-pyridyl]-1,2,4-oxadiazol- | | |
| | 5-yl]pentanoyl]piperazin-1-yl]-1-oxo-isoindolin-2-yl]-2,6- | | |
| | dioxo-1-piperidyl]methyl 2,2-dimethylpropanoate | | |
| 139 | 1-[5-chloro-6-[5-[5-[4-[2-(2,6-dioxo-3-piperidyl)-1-oxo- | 809.30 | 809 |
| | isoindolin-5-yl]piperazin-1-yl]-5-oxo-pentyl]-1,2,4-oxadiazol- | | |
| | 3-yl]-3-pyridyl]-3-(7-isopropylpyrazolo[1,5-a]pyrimidin-6- | | |
| | yl)urea | | |
| 140 | 1-[5-chloro-6-[5-[4-[4-[2-(2,6-dioxo-3-piperidyl)-1-oxo- | 795.29 | 795 |
| | isoindolin-5-yl]piperazin-1-yl]-4-oxo-butyl]-1,2,4-oxadiazol-3- | | |
| | yl]-3-pyridyl]-3-(7-isopropylpyrazolo[1,5-a]pyrimidin-6- | | |
| | yl)urea | | |
| 141 | 1-(7-cyclohexylpyrazolo[1,5-a]pyrimidin-6-yl)-3-[6-[5-[4-[4- | 801.39 | 801 |
| | [2-(2,6-dioxo-3-piperidyl)-1-oxo-isoindolin-5-yl]piperazin-1- | | |
| | yl]butyl]-1,2,4-oxadiazol-3-yl]-5-methyl-3-pyridyl]urea | | |
| 142 | 1-[6-[5-[6-[4-[2-(2,6-dioxo-3-piperidyl)-1-oxo-isoindolin-5- | 791.36 | 791 |
| | yl]piperazin-1-yl]hexyl]-1,3,4-thiadiazol-2-yl]-3-pyridyl]-3-(7- | | |
| | isopropylpyrazolo[1,5-a]pyrimidin-6-yl)urea | | |
| 143 | 1-(7-cyclopentylpyrazolo[1,5-a]pyrimidin-6-yl)-3-[6-[5-[6-[4- | 817.37 | 817 |
| | [2-(2,6-dioxo-3-piperidyl)-1-oxo-isoindolin-5-yl]piperazin-1- | | |
| | yl]hexyl]-1,3,4-thiadiazol-2-yl]-3-pyridyl]urea | | |
| 144 | 1-(7-isopropylpyrazolo[1,5-a]pyrimidin-6-yl)-3-[5-methyl-6- | 789.36 | 789 |
| | [5-[4-[4-[2-(1-methyl-2,6-dioxo-3-piperidyl)-1-oxo-isoindolin- | | |
| | 5-yl]piperazin-1-yl]-4-oxo-butyl]-1,2,4-oxadiazol-3-yl]-3- | | |
| | pyridyl]urea | | |
| 145 | 1-(7-isopropylpyrazolo[1,5-a]pyrimidin-6-yl)-3-[5-methyl-6- | 803.37 | 803 |
| | [5-[5-[4-[2-(1-methyl-2,6-dioxo-3-piperidyl)-1-oxo-isoindolin- | | |
| | 5-yl]piperazin-1-yl]-5-oxo-pentyl]-1,2,4-oxadiazol-3-yl]-3- | | |
| | pyridyl]urea | | |
| 146 | 1-(7-isopropylpyrazolo[1,5-a]pyrimidin-6-yl)-3-[5-methyl-6- | 817.39 | 817 |
| | [5-[6-[4-[2-(1-methyl-2,6-dioxo-3-piperidyl)-1-oxo-isoindolin- | | |
| | 5-yl]piperazin-1-yl]-6-oxo-hexyl]-1,2,4-oxadiazol-3-yl]-3- | | |
| | pyridyl]urea | | |
| 147 | dibutyl [3-[5-[4-[5-[3-[5-[(7-isopropylpyrazolo[1,5- | 1011.46 | 1011 |
| | a]pyrimidin-6-yl)carbamoylamino]-3-methyl-2-pyridyl]-1,2,4- | | |
| | oxadiazol-5-yl]pentanoyl]piperazin-1-yl]-1-oxo-isoindolin-2- | | |
| | yl]-2,6-dioxo-1-piperidyl]methyl phosphate | | |
| 148 | dibutyl [3-[5-[4-[6-[3-[5-[(7-isopropylpyrazolo[1,5- | 1025.48 | 1025 |
| | a]pyrimidin-6-yl)carbamoylamino]-3-methyl-2-pyridyl]-1,2,4- | | |
| | oxadiazol-5-yl]hexanoyl]piperazin-1-yl]-1-oxo-isoindolin-2- | | |
| | yl]-2,6-dioxo-1-piperidyl]methyl phosphate | | |
| 149 | [3-[5-[4-[4-[3-[5-[(7-isopropylpyrazolo[1,5-a]pyrimidin-6- | 889.41 | 889 |
| | yl)carbamoylamino]-3-methyl-2-pyridyl]-1,2,4-oxadiazol-5- | | |
| | yl]butanoyl]piperazin-1-yl]-1-oxo-isoindolin-2-yl]-2,6-dioxo-1- | | |
| | piperidyl]methyl 2,2-dimethylpropanoate | | |
| 150 | 1-(7-tert-butylpyrazolo[1,5-a]pyrimidin-6-yl)-3-[6-[5-[5-[4-[2- | 789.39 | 789 |
| | (2,6-dioxo-3-piperidyl)-1-oxo-isoindolin-5-yl]piperazin-1- | | |
| | yl]pentyl]-1,2,4-oxadiazol-3-yl]-5-methyl-3-pyridyl]urea | | |
| 151 | 1-(7-cyclopentylpyrazolo[1,5-a]pyrimidin-6-yl)-3-[1-[5-[4-[2- | 790.34 | 790 |
| | (2,6-dioxo-3-piperidyl)-1-oxo-isoindolin-5-yl]piperazin-1-yl]- | | |
| | 5-oxo-pentyl]-3-(trifluoromethyl)pyrazol-4-yl]urea | | |
| 152 | 1-(7-cyclobutylpyrazolo[1,5-a]pyrimidin-6-yl)-3-[1-[5-[4-[2- | 776.32 | 776 |
| | (2,6-dioxo-3-piperidyl)-1-oxo-isoindolin-5-yl]piperazin-1-yl]- | | |
| | 5-oxo-pentyl]-3-(trifluoromethyl)pyrazol-4-yl]urea | | |
| 153 | 1-[6-[5-[6-[4-[2-(2,6-dioxo-3-piperidyl)-1-oxo-isoindolin-5- | 819.35 | 819 |
| | yl]piperazin-1-yl]-6-oxo-hexyl]-1,3,4-thiadiazol-2-yl]-5- | | |
| | methyl-3-pyridyl]-3-(7-isopropylpyrazolo[1,5-a]pyrimidin-6- | | |
| | yl)urea | | |
| 154 | 1-[6-[5-[5-[4-[2-(2,6-dioxo-3-piperidyl)-1-oxo-isoindolin-5- | 805.34 | 805 |
| | yl]piperazin-1-yl]-5-oxo-pentyl]-1,3,4-thiadiazol-2-yl]-5- | | |
| | methyl-3-pyridyl]-3-(7-isopropylpyrazolo[1,5-a]pyrimidin-6- | | |
| | yl)urea | | |
| 155 | 1-[1-[5-[4-[2-(2,6-dioxo-3-piperidyl)-1-oxo-isoindolin-5- | 792.32 | 792 |
| | yl]piperazin-1-yl]-5-oxo-pentyl]-3-(trifluoromethyl)pyrazol-4- | | |
| | yl]-3-(7-tetrahydrofuran-2-ylpyrazolo[1,5-a]pyrimidin-6- | | |
| | yl)urea | | |
| 156 | 1-(7-cyclobutylpyrazolo[1,5-a]pyrimidin-6-yl)-3-[1-[5-[4-[2- | 762.35 | 762 |
| | (2,6-dioxo-3-piperidyl)-1-oxo-isoindolin-5-yl]piperazin-1- | | |
| | yl]pentyl]-3-(trifluoromethyl)pyrazol-4-yl]urea | | |
| 157 | 1-[1-[5-[4-[2-(2,6-dioxo-3-piperidyl)-1-oxo-isoindolin-5- | 764.32 | 764 |
| | yl]piperazin-1-yl]-5-oxo-pentyl]-3-(trifluoromethyl)pyrazol-4- | | |
| | yl]-3-(7-isopropylpyrazolo[1,5-a]pyrimidin-6-yl)urea | | |
| 158 | 1-[5-chloro-6-[5-[6-[4-[2-(2,6-dioxo-3-piperidyl)-1-oxo- | 821.30 | 821 |
| | isoindolin-5-yl]piperazin-1-yl]-6-oxo-hexyl]-1,2,4-oxadiazol-3- | | |
| | yl]-3-pyridyl]-3-(7-cyclopropylpyrazolo[1,5-a]pyrimidin-6- | | |
| | yl)urea | | |
| 159 | 1-[5-chloro-6-[5-[6-[4-[2-(2,6-dioxo-3-piperidyl)-1-oxo- | 807.32 | 807 |
| | isoindolin-5-yl]piperazin-1-yl]hexyl]-1,2,4-oxadiazol-3-yl]-3- | | |
| | pyridyl]-3-(7-cyclopropylpyrazolo[1,5-a]pyrimidin-6-yl)urea | | |
| 160 | 1-[5-chloro-6-[5-[5-[4-[2-(2,6-dioxo-3-piperidyl)-1-oxo- | 807.29 | 807 |
| | isoindolin-5-yl]piperazin-1-yl]-5-oxo-pentyl]-1,2,4-oxadiazol- | | |
| | 3-yl]-3-pyridyl]-3-(7-cyclopropylpyrazolo[1,5-a]pyrimidin-6- | | |
| | yl)urea | | |
| 161 | 1-[5-chloro-6-[5-[5-[4-[2-(2,6-dioxo-3-piperidyl)-1-oxo- | 793.31 | 793 |
| | isoindolin-5-yl]piperazin-1-yl]pentyl]-1,2,4-oxadiazol-3-yl]-3- | | |
| | pyridyl]-3-(7-cyclopropylpyrazolo[1,5-a]pyrimidin-6-yl)urea | | |
| 162 | 1-[6-[5-[6-[4-[2-(2,6-dioxo-3-piperidyl)-1-oxo-isoindolin-5- | 857.35 | 857 |
| | yl]piperazin-1-yl]-6-oxo-hexyl]-1,2,4-oxadiazol-3-yl]-5- | | |
| | (trifluoromethyl)-3-pyridyl]-3-(7-isopropylpyrazolo[1,5- | | |
| | a]pyrimidin-6-yl)urea | | |
| 163 | 1-[6-[5-[6-[4-[2-(2,6-dioxo-3-piperidyl)-1-oxo-isoindolin-5- | 843.37 | 843 |
| | yl]piperazin-1-yl]hexyl]-1,2,4-oxadiazol-3-yl]-5- | | |
| | (trifluoromethyl)-3-pyridyl]-3-(7-isopropylpyrazolo[1,5- | | |
| | a]pyrimidin-6-yl)urea | | |
| 164 | 1-[1-[5-[4-[2-(2,6-dioxo-3-piperidyl)-1-oxo-isoindolin-5- | 710.35 | 710 |
| | yl]piperazin-1-yl]-5-oxo-pentyl]-3-methyl-pyrazol-4-yl]-3-(7- | | |
| | isopropylpyrazolo[1,5-a]pyrimidin-6-yl)urea | | |
| 165 | 1-[5-chloro-6-[5-[4-[4-[2-(2,6-dioxo-3-piperidyl)-1-oxo- | 793.27 | 793 |
| | isoindolin-5-yl]piperazin-1-yl]-4-oxo-butyl]-1,2,4-oxadiazol-3- | | |
| | yl]-3-pyridyl]-3-(7-cyclopropylpyrazolo[1,5-a]pyrimidin-6- | | |
| | yl)urea | | |
| 166 | 1-[5-chloro-6-[5-[4-[4-[2-(2,6-dioxo-3-piperidyl)-1-oxo- | 779.29 | 779 |
| | isoindolin-5-yl]piperazin-1-yl]butyl]-1,2,4-oxadiazol-3-yl]-3- | | |
| | pyridyl]-3-(7-cyclopropylpyrazolo[1,5-a]pyrimidin-6-yl)urea | | |
| 167 | 1-(7-cyclopropylpyrazolo[1,5-a]pyrimidin-6-yl)-3-[6-[5-[6-[4- | 801.36 | 801 |
| | [2-(2,6-dioxo-3-piperidyl)-1-oxo-isoindolin-5-yl]piperazin-1- | | |
| | yl]-6-oxo-hexyl]-1,2,4-oxadiazol-3-yl]-5-methyl-3-pyridyl]urea | | |
| 168 | 1-(7-cyclopropylpyrazolo[1,5-a]pyrimidin-6-yl)-3-[6-[5-[6-[4- | 787.38 | 787 |
| | [2-(2,6-dioxo-3-piperidyl)-1-oxo-isoindolin-5-yl]piperazin-1- | | |
| | yl]hexyl]-1,2,4-oxadiazol-3-yl]-5-methyl-3-pyridyl]urea | | |
| 169 | 1-(7-cyclopropylpyrazolo[1,5-a]pyrimidin-6-yl)-3-[6-[5-[5-[4- | 787.34 | 787 |
| | [2-(2,6-dioxo-3-piperidyl)-1-oxo-isoindolin-5-yl]piperazin-1- | | |
| | yl]-5-oxo-pentyl]-1,2,4-oxadiazol-3-yl]-5-methyl-3- | | |
| | pyridyl]urea | | |
| 170 | 1-(7-cyclopropylpyrazolo[1,5-a]pyrimidin-6-yl)-3-[6-[5-[5-[4- | 773.36 | 773 |
| | [2-(2,6-dioxo-3-piperidyl)-1-oxo-isoindolin-5-yl]piperazin-1- | | |
| | yl]pentyl]-1,2,4-oxadiazol-3-yl]-5-methyl-3-pyridyl]urea | | |
| 171 | 1-(7-cyclopropylpyrazolo[1,5-a]pyrimidin-6-yl)-3-[6-[5-[4-[4- | 773.33 | 773 |
| | [2-(2,6-dioxo-3-piperidyl)-1-oxo-isoindolin-5-yl]piperazin-1- | | |
| | yl]-4-oxo-butyl]-1,2,4-oxadiazol-3-yl]-5-methyl-3-pyridyl]urea | | |
| 172 | 1-(7-cyclopropylpyrazolo[1,5-a]pyrimidin-6-yl)-3-[6-[5-[4-[4- | 759.35 | 759 |
| | [2-(2,6-dioxo-3-piperidyl)-1-oxo-isoindolin-5-yl]piperazin-1- | | |
| | yl]butyl]-1,2,4-oxadiazol-3-yl]-5-methyl-3-pyridyl]urea | | |
| 173 | 1-[5-chloro-6-[5-[4-[4-[2-(2,6-dioxo-3-piperidyl)-1-oxo- | 835.32 | 835 |
| | isoindolin-5-yl]piperazine-1-carbonyl]cyclohexyl]-1,2,4- | | |
| | oxadiazol-3-yl]-3-pyridyl]-3-(7-isopropylpyrazolo[1,5- | | |
| | a]pyrimidin-6-yl)urea | | |
| 174 | 1-[5-chloro-6-[5-[4-[[4-[2-(2,6-dioxo-3-piperidyl)-1-oxo- | 821.34 | 821 |
| | isoindolin-5-yl]piperazin-1-yl]methyl]cyclohexyl]-1,2,4- | | |
| | oxadiazol-3-yl]-3-pyridyl]-3-(7-isopropylpyrazolo[1,5- | | |
| | a]pyrimidin-6-yl)urea | | |
| 175 | 1-[5-chloro-6-[5-[4-[4-[2-(2,6-dioxo-3-piperidyl)-1-oxo- | 849.34 | 849 |
| | isoindolin-5-yl]piperazine-1-carbonyl]cyclohexyl]methyl]- | | |
| | 1,2,4-oxadiazol-3-yl]-3-pyridyl]-3-(7-isopropylpyrazolo[1,5- | | |
| | a]pyrimidin-6-yl)urea | | |
| 176 | 1-[5-chloro-6-[5-[[4-[[4-[2-(2,6-dioxo-3-piperidyl)-1-oxo- | 835.36 | 835 |
| | isoindolin-5-yl]piperazin-1-yl]methyl]cyclohexyl]methyl]- | | |
| | 1,2,4-oxadiazol-3-yl]-3-pyridyl]-3-(7-isopropylpyrazolo[1,5- | | |
| | a]pyrimidin-6-yl)urea | | |

### Synthesis of the Representative Examples of the Compound

### Example 1. N-{6-[5-(6-{4-[2-(2,6-Dioxopiperidin-3-yl)-l-oxo-2,3-dihydro-1H-isoindol-5-yl]piperazin-1-yl}-6-oxohexyl)-1,2,4-oxadiazol-3-yl]-5-methylpyridin-3-yl}-N′-[7-(tetrahydrofuran-2-yl)pyrazolo[1,5-a]pyrimidin-6-yl]urea (Compound 2)

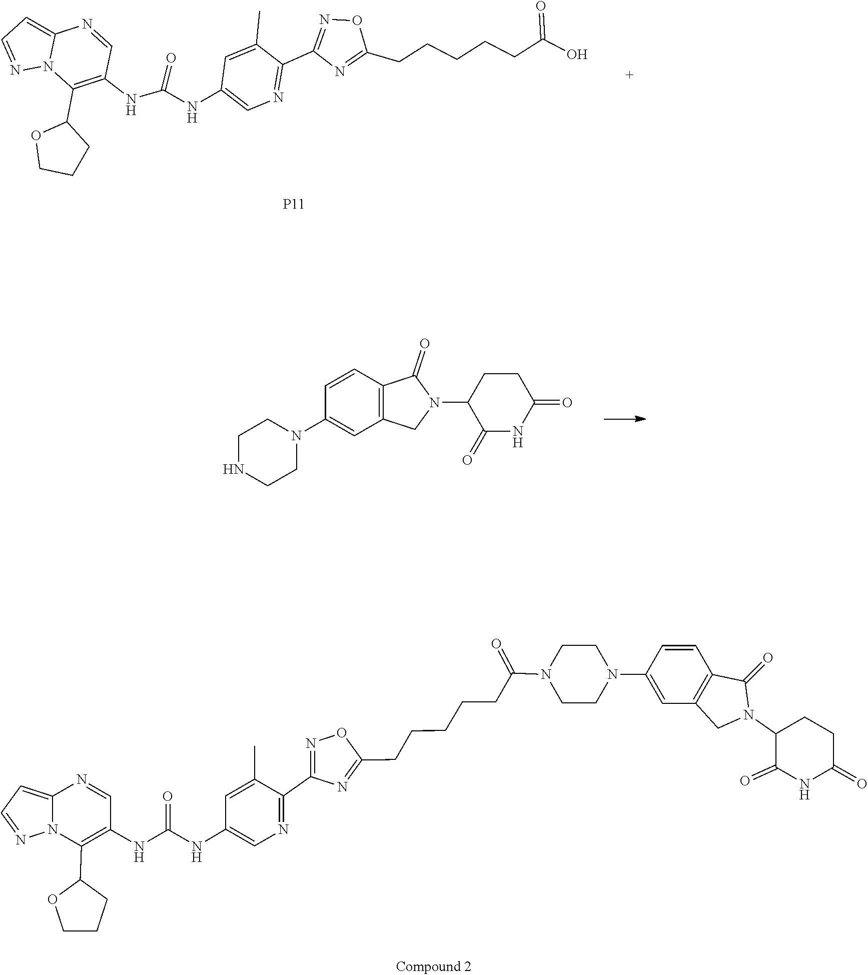 [SMILES: Cc1cc(NC(=O)Nc2cnc3ccnn3c2C2CCCO2)cnc1-c1noc(CCCCCC(=O)O)n1 ; "P11" ; O=C1CCC(N2Cc3cc(N4CCNCC4)ccc3C2=O)C(=O)N1 ; Cc1cc(NC(=O)Nc2cnc3ccnn3c2C2CCCO2)cnc1-c1noc(CCCCCC(=O)N2CCN(c3ccc4c(c3)CN(C3CCC(=O)NC3=O)C4=O)CC2)n1]

To a solution of Compound P11 (0.36 g, 0.7 mmol) in 5 mL of DMF, DIPEA (0.6 mL, 3.50 mmol) was added. The mixture was stirred at rt for 10 min. After this, HATU (0.29 g, 0.84 mmol) and 3-(1-oxo-5-piperazin-1-yl-isoindolin-2-yl)piperidine-2,6-dione hydrochloride (0.25 g, 0.7 mmol) were added. The mixture was stirred at rt for 15 h and was evaporated. The residue was purified by prep RP-HPLC on a C₁₈ column eluting with a gradient MeCN-H₂O+0.1% TFA. Yield of Compound 2 (0.135 g, 24%). ¹H NMR (400 MHz, DMSO-d₆), δ: 10.94 (s, 1H), 9.82 (s, 1H), 8.70 (s, 1H), 8.62 (d, J=2.1 Hz, 1H), 8.51 (s, 1H), 8.21 (d, J=2.2 Hz, 1H), 8.00 (s, 1H), 7.53 (d, J=8.4 Hz, 1H), 7.06 (d, J=9.0 Hz, 2H), 6.77 (d, J=2.3 Hz, 1H), 5.75 (t, J=7.2 Hz, 1H), 5.04 (dd, J=13.2, 5.0 Hz, 1H), 4.46-4.10 (m, 4H), 4.04-3.84 (m, 1H), 3.60 (s, 4H), 3.26 (s, 2H), 3.01 (t, J=7.4 Hz, 2H), 2.97-2.81 (m, 1H), 2.64-2.52 (m, 4H), 2.38 (t, J=7.3 Hz, 4H), 2.06 (s, 4H), 2.00-1.90 (m, 1H), 1.81 (dd, J=14.9, 7.5 Hz, 2H), 1.65-1.52 (m, 2H), 1.42 (d, J=7.1 Hz, 2H).

### Example 2. N-{5-Chloro-6-[5-(6-{4-[2-(2,6-dioxopiperidin-3-yl)-1-oxo-2,3-dihydro-1H-isoindol-5-yl]piperazin-1-yl}-6-oxohexyl)-1,2,4-oxadiazol-3-yl]pyridin-3-yl}-N′-(7-cyclopentylpyrazolo[1,5-a]pyrimidin-6-yl)urea (Compound 3)

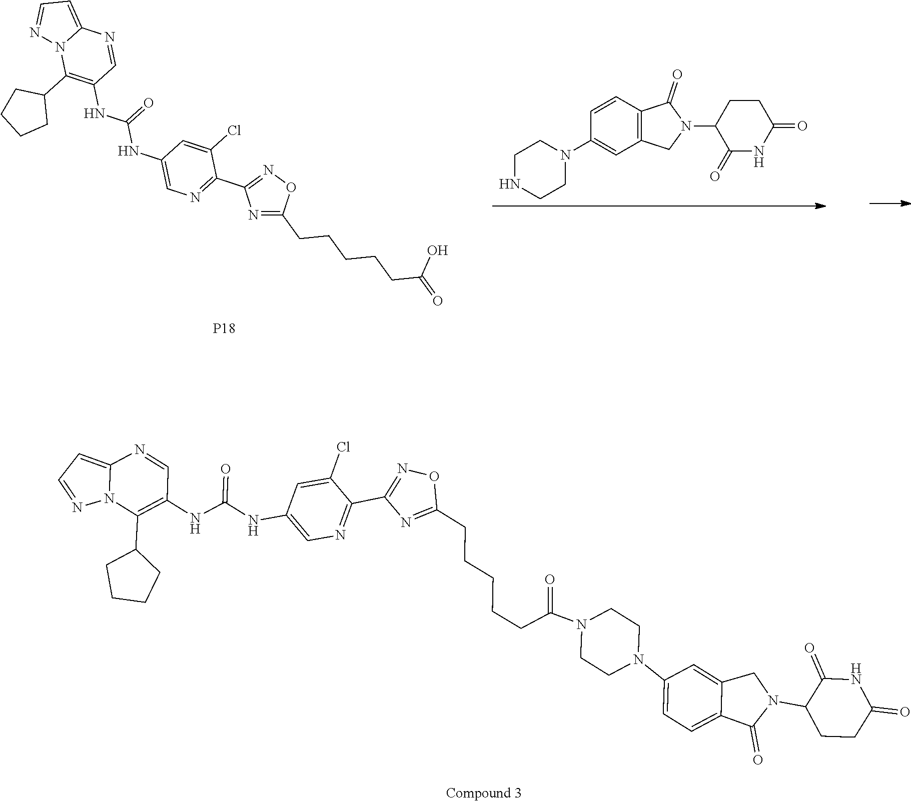 [SMILES: O=C(O)CCCCCc1nc(-c2ncc(NC(=O)Nc3cnc4ccnn4c3C3CCCC3)cc2Cl)no1 ; "P18" ; O=C1CCC(N2Cc3cc(N4CCNCC4)ccc3C2=O)C(=O)N1 ; O=C1CCC(N2Cc3cc(N4CCN(C(=O)CCCCCc5nc(-c6ncc(NC(=O)Nc7cnc8ccnn8c7C7CCCC7)cc6Cl)no5)CC4)ccc3C2=O)C(=O)N1]

N-ethyl-N-isopropylpropan-2-amine (0.042 g, 0.325 mmol) was added to solution of 6-{3-[3-chloro-5-({[(7-cyclopentylpyrazolo[1,5-a]pyrimidin-6-yl)amino]carbonyl}amino)pyridin-2-yl]-1,2,4-oxadiazol-5-yl}hexanoic acid P18 (0.035 g, 0.065 mmol) in DMF and the mixture was stirred for 5 min. 1-[Bis(dimethylamino)methylene]-1H-1,2,3-triazolo[4,5-b]pyridinium 3-oxide hexafluorophosphate (0.027 g, 0.072 mmol) was added and the mixture was stirred for 5 min. 3-(1-oxo-5-piperazin-1-yl-1,3-dihydro-2H-isoindol-2-yl)piperidine-2,6-dione hydrochloride (0.024 g, 0.065 mmol) was added into the flask and stirred overnight at ambient temperature. The obtained crude residue was purified by RP-HPLC on a C18 column eluting with a gradient MeCN-H₂O+0.1% TFA to provide N-{5-chloro-6-[5-(6-{4-[2-(2,6-dioxopiperidin-3-yl)-1-oxo-2,3-dihydro-1H-isoindol-5-yl]piperazin-1-yl}-6-oxohexyl)-1,2,4-oxadiazol-3-yl]pyridin-3-yl}-N′-(7-cyclopentylpyrazolo[1,5-a]pyrimidin-6-yl)urea (Compound 3, 0.0078 g, 14%). ¹H NMR (400 MHz, DMSO-d₆), δ: 10.94 (s, 1H), 9.66 (s, 1H), 8.70 (d, J=2.1 Hz, 2H), 8.48 (s, 1H), 8.35 (d, J=2.2 Hz, 1H), 8.24 (d, J=2.3 Hz, 1H), 7.53 (d, J=8.4 Hz, 1H), 7.06 (d, J=8.7 Hz, 2H), 6.76 (d, J=2.4 Hz, 1H), 5.05 (dd, J=13.3, 5.1 Hz, 2H), 4.27 (dd, J=49.3, 17.0 Hz, 4H), 3.85 (p, J=9.1 Hz, 2H), 3.60 (s, 3H), 3.27 (s, 2H), 3.03 (t, J=7.4 Hz, 2H), 2.97-2.81 (m, 2H), 2.57 (d, J=24.3 Hz, 1H), 2.38 (t, J=7.4 Hz, 4H), 1.98 (s, 2H), 1.81 (dt, J=15.2, 7.7 Hz, 4H), 1.70 (d, J=5.3 Hz, 2H), 1.57 (dd, J=14.7, 7.4 Hz, 2H), 1.48-1.34 (m, 2H).

### Example 3. N-(7-Cyclopentylpyrazolo[1,5-a]pyrimidin-6-yl)-N′-{6-[5-(6-{4-[2-(2,6-dioxopiperidin-3-yl)-1-oxo-2,3-dihydro-1H-isoindol-5-yl]piperazin-1-yl}-6-oxohexyl)-1,3,4-oxadiazol-2-yl]-5-methylpyridin-3-yl}urea (Compound 5)

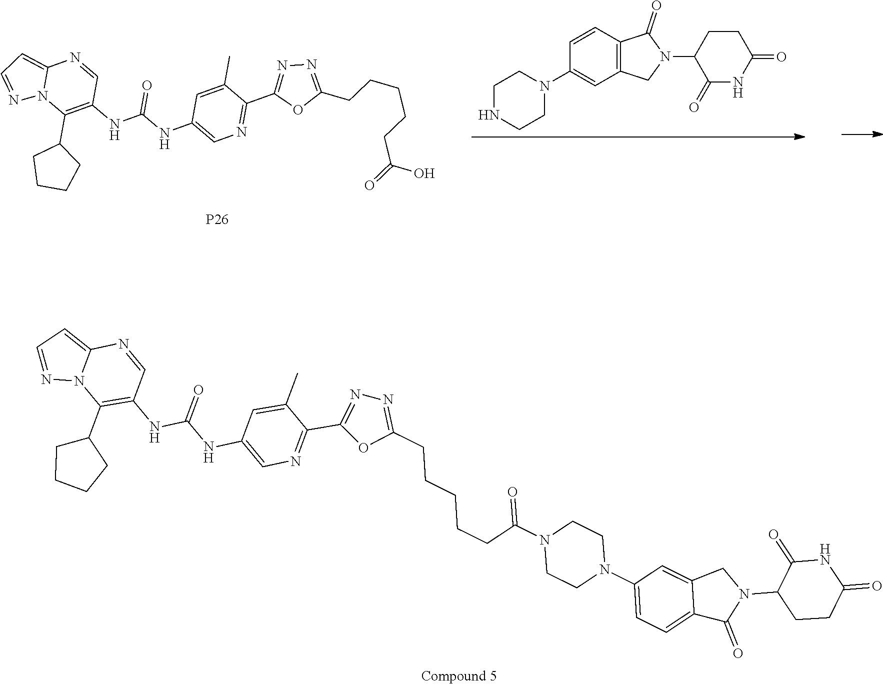

N-Ethyl-N-isopropylpropan-2-amine (0.066 g, 0.5 mmol) was added to solution of 6-{5-[5-({[(7-cyclopentylpyrazolo[1,5-a]pyrimidin-6-yl)amino]carbonyl}amino)-3-methylpyridin-2-yl]-1,3,4-oxadiazol-2-yl}hexanoic acid P26 (0.053 g, 0.1 mmol) in DMF and the mixture was stirred for 5 min. 1-[bis(dimethylamino)methylene]-1H-1,2,3-triazolo[4,5-b]pyridinium 3-oxide hexafluorophosphate (0.043 g, 0.11 mmol) was added and the mixture was stirred for 5 min. 3-(1-oxo-5-piperazin-1-yl-1,3-dihydro-2H-isoindol-2-yl)piperidine-2,6-dione hydrochloride (0.037 g, 0.1 mmol) was added into the flask and stirred overnight at ambient temperature. The obtained crude residue was purified by RP-HPLC on a C18 column eluting with a gradient MeCN-H₂O+0.1% TFA to provide N-(7-cyclopentylpyrazolo[1,5-a]pyrimidin-6-yl)-N′-{6-[5-(6-{4-[2-(2,6-dioxopiperidin-3-yl)-1-oxo-2,3-dihydro-1H-isoindol-5-yl]piperazin-1-yl}-6-oxohexyl)-1,3,4-oxadiazol-2-yl]-5-methylpyridin-3-yl}urea—Compound 5 (0.031 g, 36%). ¹H NMR (400 MHz, DMSO-d₆), δ: 10.92 (s, 1H), 9.47 (s, 1H), 8.66 (s, 1H), 8.58 (s, 1H), 8.48 (s, 1H), 8.23 (s, 1H), 8.03 (s, 1H), 7.54 (d, J=8.3 Hz, 1H), 7.06 (d, J=9.4 Hz, 2H), 6.75 (d, J=1.9 Hz, 1H), 5.04 (dd, J=13.1, 5.1 Hz, 1H), 4.27 (dd, J=48.0, 16.8 Hz, 2H), 3.94-3.82 (m, 2H), 3.61 (s, 6H), 3.29 (d, J=20.5 Hz, 2H), 2.95 (t, J=7.3 Hz, 2H), 2.69-2.56 (m, 4H), 2.37 (d, J=7.4 Hz, 4H), 1.98 (s, 4H), 1.86 (s, 2H), 1.81-1.74 (m, 2H), 1.71 (s, 2H), 1.57 (d, J=7.2 Hz, 2H), 1.41 (s, 2H).

### Example 4. N-(7-Cyclopentylpyrazolo[1,5-a]pyrimidin-6-yl)-N′-{6-[1-(7-{4-[2-(2,6-dioxopiperidin-3-yl)-1-oxo-2,3-dihydro-1H-isoindol-5-yl]piperazin-1-yl}-7-oxoheptyl)-1H-1,2,3-triazol-4-yl]-5-methylpyridin-3-yl}urea (Compound 6)

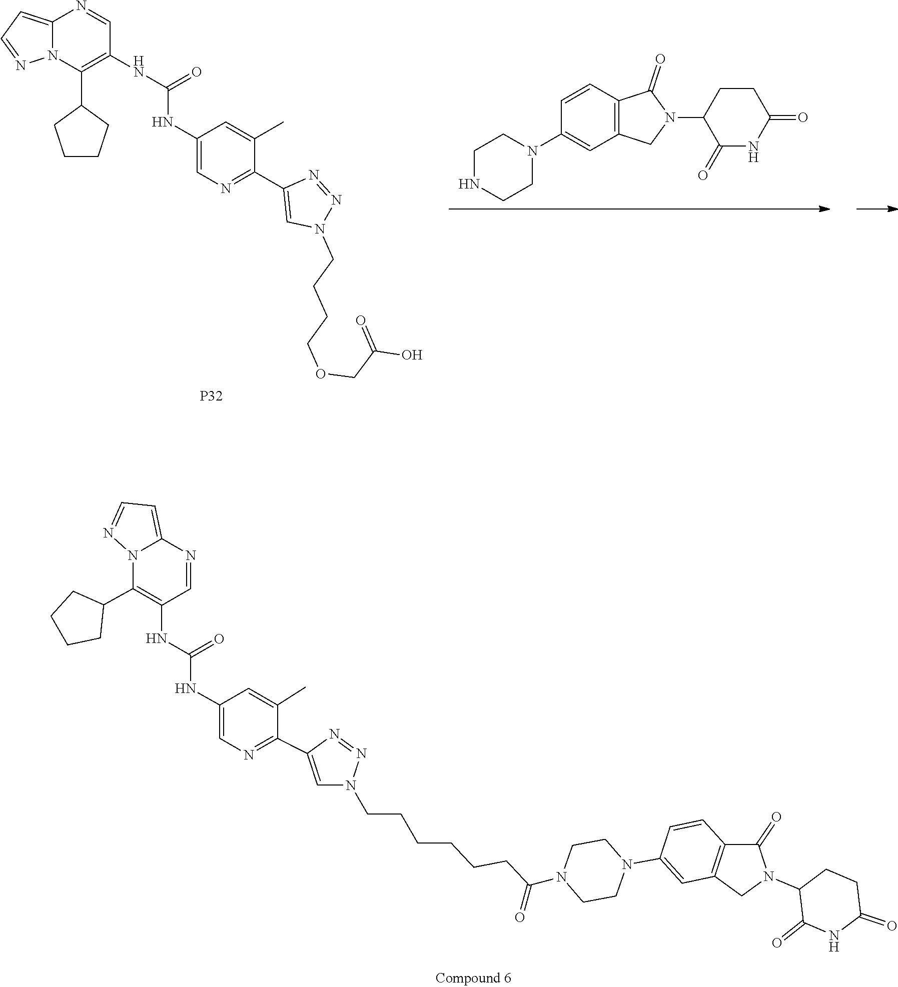 [SMILES: Cc1cc(NC(=O)Nc2cnc3ccnn3c2C2CCCC2)cnc1-c1cn(CCCCOCC(=O)O)nn1 ; "P32" ; O=C1CCC(N2Cc3cc(N4CCNCC4)ccc3C2=O)C(=O)N1 ; Cc1cc(NC(=O)Nc2cnc3ccnn3c2C2CCCC2)cnc1-c1cn(CCCCCCC(=O)N2CCN(c3ccc4c(c3)CN(C3CCC(=O)NC3=O)C4=O)CC2)nn1]

N-Ethyl-N-isopropylpropan-2-amine (0.168 g, 1.3 mmol) was added to solution of 7-{4-[5-({[(7-cyclopentylpyrazolo[1,5-a]pyrimidin-6-yl)amino]carbonyl}amino)-3-methylpyridin-2-yl]-1H-1,2,3-triazol-1-yl}heptanoic acid P32 (0.138 g, 0.26 mmol) in DMF and the mixture was stirred for 5 min. 1-[Bis(dimethylamino)methylene]-1H-1,2,3-triazolo[4,5-b]pyridinium 3-oxide hexafluorophosphate (0.109 g, 0.29 mmol) was added and the mixture was stirred for 5 min. 3-(1-oxo-5-piperazin-1-yl-1,3-dihydro-2H-isoindol-2-yl)piperidine-2,6-dione hydrochloride (0.095 g, 0.26 mmol) was added into the flask and stirred overnight at ambient temperature. The obtained crude residue was purified by RP-HPLC on a C18 column eluting with a gradient MeCN-H₂O+0.1% TFA to provide N-(7-cyclopentylpyrazolo[1,5-a]pyrimidin-6-yl)-N′-{6-[1-(7-{4-[2-(2,6-dioxopiperidin-3-yl)-1-oxo-2,3-dihydro-1H-isoindol-5-yl]piperazin-1-yl}-7-oxoheptyl)-1H-1,2,3-triazol-4-yl]-5-methylpyridin-3-yl}urea—Compound 6 (0.0248 g, 11%). ¹H NMR (400 MHz, DMSO-d₆), δ: 10.92 (s, 1H), 9.32 (s, 1H), 8.65 (s, 1H), 8.58 (s, 1H), 8.56 (s, 1H), 8.47 (s, 1H), 8.23 (d, J=2.4 Hz, 1H), 7.97 (s, 1H), 7.54 (d, J=8.4 Hz, 1H), 7.06 (d, J=9.4 Hz, 2H), 6.75 (d, J=2.3 Hz, 1H), 5.04 (dd, J=13.3, 5.1 Hz, 1H), 4.44 (t, J=6.9 Hz, 2H), 4.28 (dd, J=47.7, 17.0 Hz, 2H), 3.86 (d, J=8.8 Hz, 4H), 3.59 (s, 4H), 3.29 (d, J=18.5 Hz, 4H), 3.00-2.76 (m, 1H), 2.70-2.56 (m, 5H), 2.34 (t, J=7.1 Hz, 4H), 1.99 (s, 2H), 1.93-1.80 (m, 4H), 1.71 (s, 2H), 1.51 (d, J=7.7 Hz, 2H), 1.31 (s, 2H).

### Example 5. N-(7-cyclopentylpyrazolo[1,5-a]pyrimidin-6-yl)-N′-{6-[5-(4-{4-[2-(2,6-dioxopiperidin-3-yl)-1-oxo-2,3-dihydro-1H-isoindol-5-yl]piperazin-1-yl}-4-oxobutyl)-1,2,4-oxadiazol-3-yl]-5-methylpyridin-3-yl}urea (Compound 12)

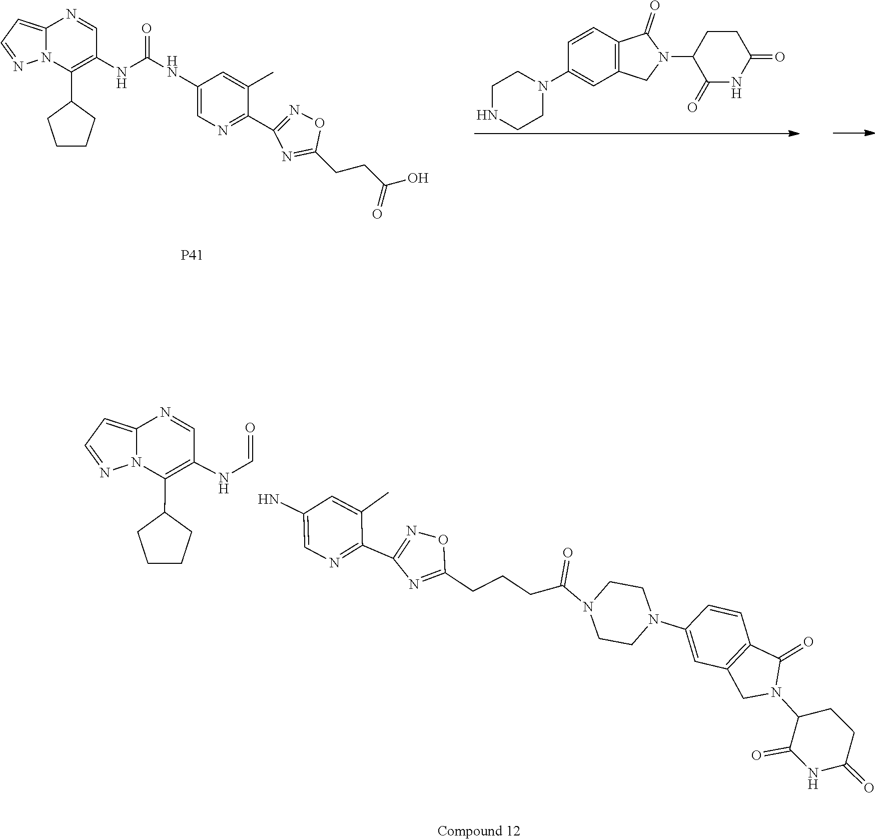

To a solution of Compound P41 (0.24 g, 0.5 mmol) in 5 mL DMF, DIPEA (0.6 mL, 3.50 mmol) was added. The mixture was stirred at rt for 10 min. Then, HATU (0.22 g, 0.6 mmol) and 3-(1-oxo-5-piperazin-1-yl-isoindolin-2-yl)piperidine-2,6-dione hydrochloride (0.18 g, 0.5 mmol) were added. The mixture was stirred at rt for 15 h and was evaporated. The residue was purified by prep RP-HPLC on a C18 column eluting with a gradient MeCN-H₂O+0.1% TFA. Yield of compound 12 (0.04 g, 11%). ¹H NMR (400 MHz, DMSO-d₆), δ: 10.94 (s, 1H), 9.37 (s, 1H), 8.62 (d, J=2.2 Hz, 1H), 8.53 (s, 1H), 8.48 (s, 1H), 8.23 (d, J=2.4 Hz, 1H), 7.99 (d, J=1.9 Hz, 1H), 7.54 (d, J=8.4 Hz, 1H), 7.07 (d, J=9.2 Hz, 3H), 6.75 (d, J=2.3 Hz, 1H), 5.05 (dd, J=13.3, 5.1 Hz, 2H), 4.28 (dd, J=49.7, 16.9 Hz, 2H), 3.85 (dd, J=18.1, 9.0 Hz, 2H), 3.62 (s, 6H), 3.34 (m, 1H), 3.32 (s, 3H), 3.06 (t, J=7.4 Hz, 2H), 2.88 (dd, J=21.3, 9.2 Hz, 2H), 2.63-2.53 (m, 2H), 2.32 (t, J=27.0 Hz, 2H), 2.04 (d, J=7.4 Hz, 2H), 1.98 (s, 2H), 1.86 (s, 2H), 1.70 (d, J=4.9 Hz, 2H).

### Example 6. N-(7-Cyclopentylpyrazolo[1,5-a]pyrimidin-6-yl)-N′-{6-[5-(3-{4-[2-(2,6-dioxopiperidin-3-yl)-1-oxo-2,3-dihydro-1H-isoindol-5-yl]piperazin-1-yl}-3-oxopropyl)-1,2,4-oxadiazol-3-yl]-5-methylpyridin-3-yl}urea (Compound 13)

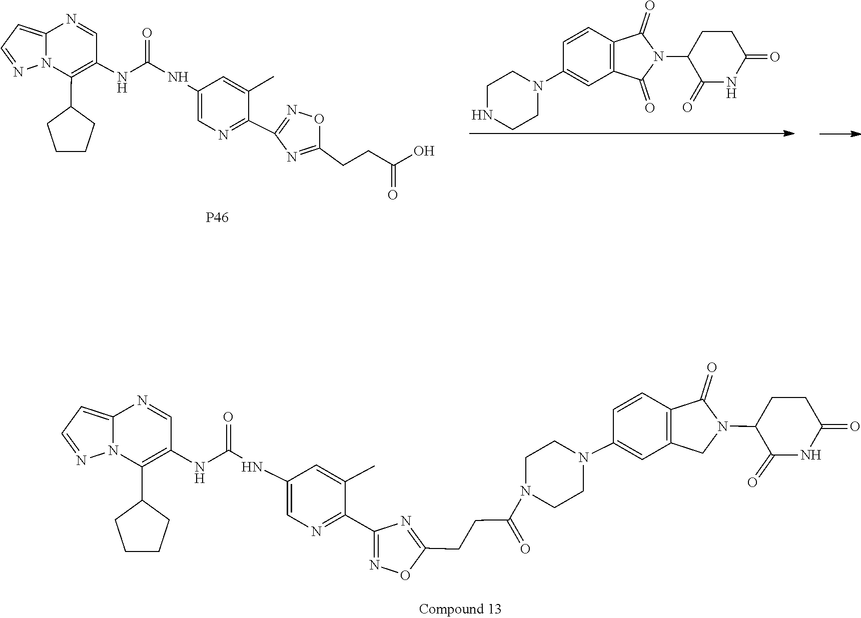

To a solution of Compound P46 (0.32 g, 0.67 mmol) in 5 mL DMF, DIPEA (0.82 mL, 2.68 mmol) was added. The mixture was stirred at rt for 10 min. After this, HATU (0.31 g, 0.80 mmol) and 3-(1-oxo-5-piperazin-1-yl-isoindolin-2-yl)piperidine-2,6-dione hydrochloride (0.25 g, 0.67 mmol) were added. The mixture was stirred at rt for 15 h and was evaporated. The residue was purified by prep RP-HPLC on a C₁₈ column eluting with a gradient MeCN-H₂O+0.1% TFA. Yield of compound 13 (0.025 g, 5%). ¹H NMR (400 MHz, DMSO-d₆), δ: 10.94 (s, 1H), 9.39 (s, 1H), 8.62 (s, 1H), 8.55 (s, 1H), 8.48 (s, 1H), 8.23 (d, J=2.2 Hz, 1H), 7.99 (s, 1H), 7.54 (d, J=8.5 Hz, 1H), 7.08 (d, J=9.3 Hz, 2H), 6.75 (d, J=2.2 Hz, 1H), 5.05 (dd, J=12.9, 4.8 Hz, 1H), 4.24 (s, 6H), 3.91-3.82 (m, 1H), 3.64 (d, J=24.6 Hz, 2H), 3.38 (s, 2H), 3.29 (s, 2H), 3.21 (d, J=6.8 Hz, 2H), 3.03 (d, J=6.5 Hz, 2H), 2.91 (d, J=12.7 Hz, 1H), 2.58 (d, J=12.1 Hz, 1H), 2.54 (s, 3H), 2.35 (s, 2H), 1.98 (s, 2H), 1.86 (s, 2H), 1.71 (s, 2H).

### Example 7. N-(7-Cyclopentylpyrazolo[1,5-a]pyrimidin-6-yl)-N′-{6-[5-(5-{4-[2-(2,6-dioxopiperidin-3-yl)-1-oxo-2,3-dihydro-1H-isoindol-5-yl]piperazin-1-yl}pentyl)-1,2,4-oxadiazol-3-yl]-5-methylpyridin-3-yl}urea (Compound 17)

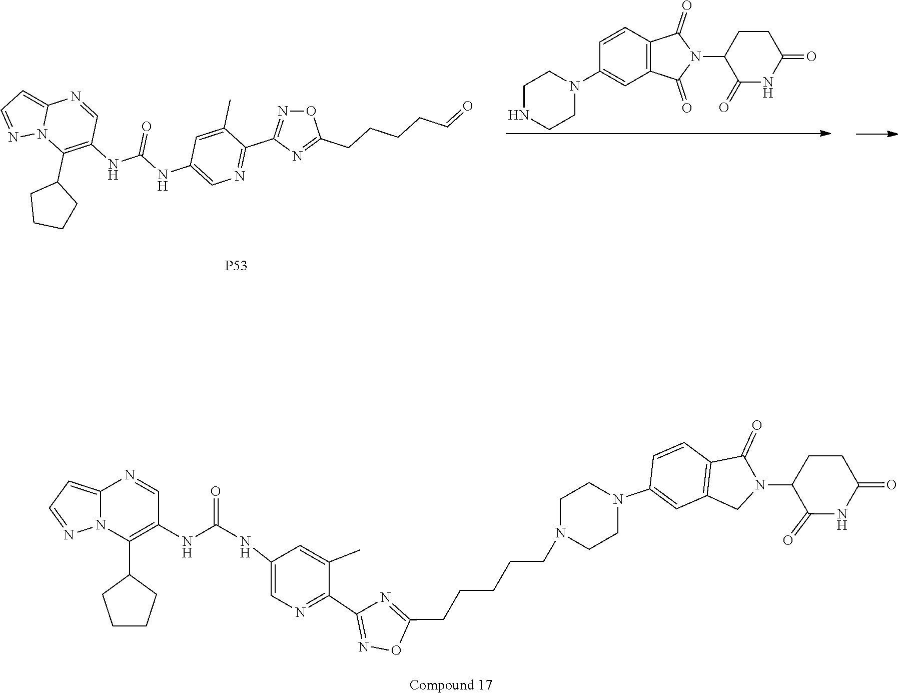

A solution of Compound P53 (0.18 g, 0.37 mmol), 3-(1-oxo-5-piperazin-1-yl-isoindolin-2-yl)piperidine-2,6-dione hydrochloride (0.13 g, 0.37 mmol) and acetic acid, potassium salt (0.07 g, 0.74 mmol) in THF (6.0 mL) was stirred at ambient temperature for 1 h. Then, sodium triacetoxyborohydride (0.16 g, 0.74 mmol) was added and the mixture was stirred at ambient temperature for 15 h. The mixture was filtered through Celite and evaporated. The residue was purified by prep RP-HPLC on a C₁₈ column eluting with a gradient MeCN-H₂O+0.1% TFA. Yield of compound 17 (0.09 g, 31%). ¹H NMR (400 MHz, DMSO-d₆), δ: 10.95 (s, 1H), 9.51 (s, 1H), 8.68 (s, 1H), 8.65 (d, J=2.1 Hz, 1H), 8.47 (s, 1H), 8.23 (d, J=2.3 Hz, 1H), 7.99 (d, J=1.9 Hz, 1H), 7.59 (d, J=8.4 Hz, 1H), 7.14 (dd, J=19.3, 10.8 Hz, 2H), 6.75 (d, J=2.3 Hz, 1H), 5.06 (dd, J=13.2, 5.1 Hz, 1H), 4.29 (dd, J=48.3, 17.0 Hz, 2H), 4.02 (d, J=10.4 Hz, 2H), 3.93-3.80 (m, 1H), 3.61 (d, J=9.7 Hz, 2H), 3.13 (t, J=12.7 Hz, 6H), 3.06 (t, J=7.4 Hz, 2H), 2.91 (t, J=12.7 Hz, 2H), 2.71-2.57 (m, 2H), 2.54 (s, 3H), 2.43-2.27 (m, 2H), 1.98 (s, 2H), 1.93-1.81 (m, 4H), 1.79-1.60 (m, 4H), 1.45 (d, J=7.5 Hz, 2H).

### Example 8. N-(7-Cyclopentylpyrazolo[1,5-a]pyrimidin-6-yl)-N′-{6-[5-(3-{4-[2-(2,6-dioxopiperidin-3-yl)-1-oxo-2,3-dihydro-1H-isoindol-5-yl]piperazin-1-yl}propyl)-1,2,4-oxadiazol-3-yl]-5-methylpyridin-3-yl}urea (Compound 18)

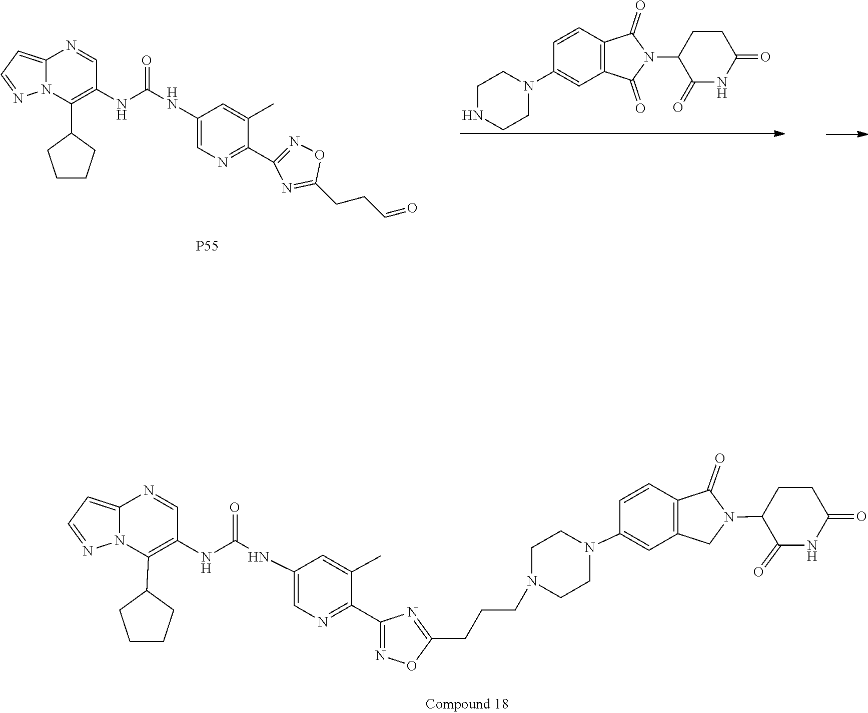

A solution of Compound P55 (0.10 g, 0.22 mmol), 3-(1-oxo-5-piperazin-1-yl-isoindolin-2-yl)piperidine-2,6-dione hydrochloride (0.08 g, 0.22 mmol) and acetic acid, potassium salt (0.05 g, 0.44 mmol) in THF (6.0 mL) was stirred at ambient temperature for 1 h. Then, sodium triacetoxyborohydride (0.10 g, 0.44 mmol) was added and the mixture was stirred at ambient temperature for 15 h. The mixture was filtered through Celite and evaporated. The residue was purified by prep RP-HPLC on a C18 column eluting with a gradient MeCN-H₂O+0.1% TFA. Yield of compound 18 on 2 steps (P55-Compound 18): 0.027 g, 16%. ¹H NMR (400 MHz, DMSO-d₆), δ: 10.93 (s, 1H), 9.36 (s, 1H), 8.62 (d, J=2.1 Hz, 1H), 8.52 (s, 1H), 8.48 (s, 1H), 8.23 (d, J=2.3 Hz, 1H), 7.98 (d, J=1.9 Hz, 1H), 7.50 (d, J=8.4 Hz, 1H), 7.03 (d, J=8.5 Hz, 2H), 6.75 (d, J=2.3 Hz, 1H), 5.04 (dd, J=13.1, 5.1 Hz, 1H), 4.26 (dd, J=50.1, 16.9 Hz, 2H), 3.98-3.74 (m, 6H), 3.22 (m, 4H), 3.05 (t, J=7.0 Hz, 2H), 2.88 (dd, J=21.7, 9.2 Hz, 1H), 2.63 (d, J=33.6 Hz, 2H), 2.45 (s, 3H), 2.41-2.24 (m, 4H), 1.98 (dd, J=26.6, 19.5 Hz, 4H), 1.88 (d, J=18.7 Hz, 2H), 1.70 (d, J=4.8 Hz, 2H).

### Example 9. N-(3-Chloro-7-cyclopentylpyrazolo[1,5-a]pyrimidin-6-yl)-N′-{6-[5-(3-{4-[2-(2,6-dioxopiperidin-3-yl)-1-oxo-2,3-dihydro-1H-isoindol-5-yl]piperazin-1-yl}propyl)-1,2,4-oxadiazol-3-yl]-5-methylpyridin-3-yl}urea (Compound 19)

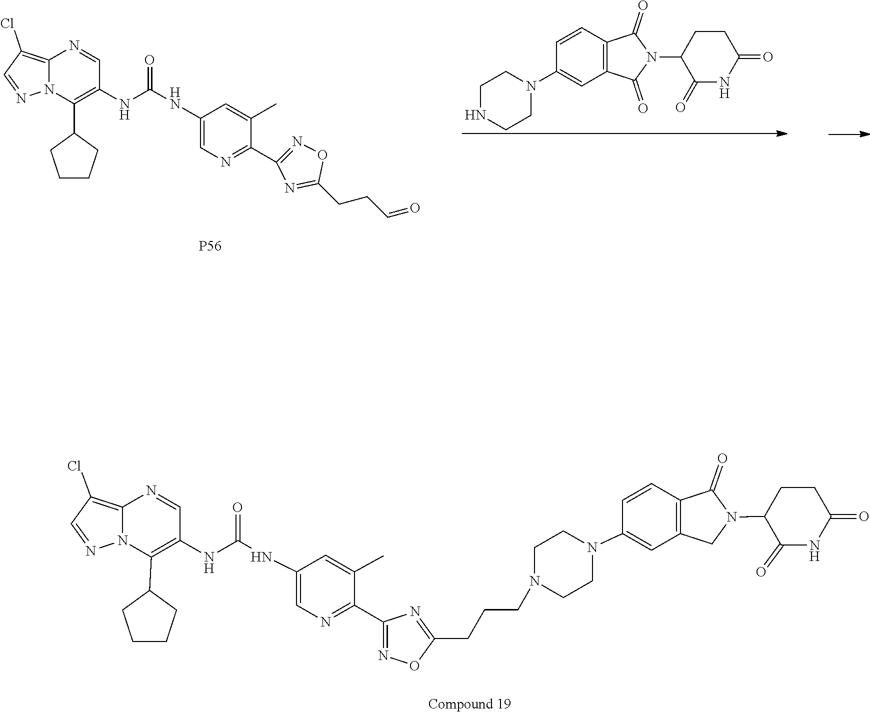

A solution of P56 (0.10 g, 0.22 mmol), 3-(1-oxo-5-piperazin-1-yl-isoindolin-2-yl)piperidine-2,6-dione hydrochloride (0.08 g, 0.22 mmol) and acetic acid, potassium salt (0.05 g, 0.44 mmol) in THF (6.0 mL) was stirred at ambient temperature for 1 h. After this, sodium triacetoxyborohydride (0.10 g, 0.44 mmol) was added and the mixture was stirred at ambient temperature for 15 h. The mixture was filtered through Celite and evaporated. The residue was purified by prep RP-HPLC on a C18 column eluting with a gradient MeCN-H₂O+0.1% TFA to yield 0.027 g of compound 19, 16% on 2 steps (P56 and Compound 19). ¹H NMR (400 MHz, DMSO-d₆), δ: 10.93 (s, 1H), 9.34 (s, 1H), 8.61 (s, 2H), 8.59 (s, 1H), 8.40 (s, 1H), 7.98 (s, 1H), 7.50 (d, J=8.4 Hz, 1H), 7.02 (d, J=8.7 Hz, 2H), 5.03 (dd, J=13.3, 4.9 Hz, 1H), 4.26 (dd, J=49.5, 16.8 Hz, 2H), 3.92-3.66 (m, 1H), 3.22 (m, 4H), 3.05 (t, J=7.0 Hz, 2H), 2.89 (t, J=12.9 Hz, 2H), 2.63 (d, J=32.1 Hz, 2H), 2.46 (s, 3H), 2.41-2.21 (m, 6H), 2.00 (dd, J=13.4, 6.9 Hz, 6H), 1.89 (d, J=10.7 Hz, 2H), 1.71 (s, 2H).

### Example 10. N-(3-chloro-7-cyclopentylpyrazolo[1,5-a]pyrimidin-6-yl)-N′-{6-[5-(4-{4-[2-(2,6-dioxopiperidin-3-yl)-1-oxo-2,3-dihydro-1H-isoindol-5-yl]piperazin-1-yl}butyl)-1,2,4-oxadiazol-3-yl]-5-methylpyridin-3-yl}urea (Compound 20)

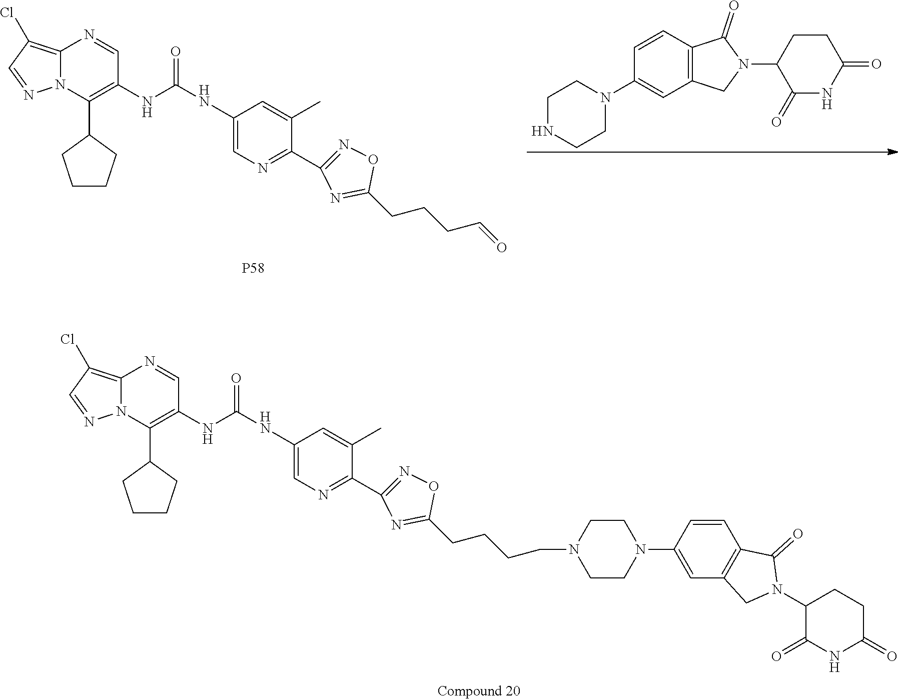

A solution of Compound P58 (0.1 g, 0.2 mmol), 3-(1-oxo-5-piperazin-1-yl-isoindolin-2-yl)piperidine-2,6-dione hydrochloride (0.08 g, 0.2 mmol) and potassium acetate (0.04 g, 0.4 mmol) in THF (6.0 mL) was stirred at ambient temperature for 1 h. Then, sodium triacetoxyborohydride (0.09 g, 0.4 mmol) was added and the mixture was stirred at ambient temperature for 15 h. The mixture was filtered through Celite and evaporated. The residue was purified by prep RP-HPLC on a C₁₈ column eluting with a gradient MeCN-H₂O+0.1% TFA. Yield of compound 20: 0.017 g, 11% on 2 steps (P58 and Compound 20). ¹H NMR (400 MHz, DMSO-d₆), δ: 10.93 (s, 1H), 9.36 (s, 1H), 8.62 (d, J=2.3 Hz, 2H), 8.59 (s, 1H), 8.40 (s, 1H), 8.00 (s, 1H), 7.51 (d, J=8.5 Hz, 1H), 7.05 (d, J=8.4 Hz, 2H), 5.04 (dd, J=13.4, 4.9 Hz, 1H), 4.26 (dd, J=49.6, 17.0 Hz, 2H), 3.87 (dd, J=18.0, 9.0 Hz, 1H), 3.28 (s, 4H), 3.05 (t, J=7.4 Hz, 2H), 2.88 (dd, J=21.6, 9.7 Hz, 2H), 2.58 (d, J=15.4 Hz, 2H), 2.49 (d, J=8.3 Hz, 3H), 2.37 (dd, J=12.9, 7.2 Hz, 2H), 2.32 (d, J=11.9 Hz, 2H), 1.96 (d, J=12.9 Hz, 4H), 1.92-1.79 (m, 6H), 1.70 (d, J=4.5 Hz, 2H), 1.64-1.55 (m, 2H).

### Example 11. N-(7-Cyclopentylpyrazolo[1,5-a]pyrimidin-6-yl)-N′-{6-[5-(4-{4-[2-(2,6-dioxopiperidin-3-yl)-1-oxo-2,3-dihydro-1H-isoindol-5-yl]piperazin-1-yl}butyl)-1,2,4-oxadiazol-3-yl]-5-methylpyridin-3-yl}urea (Compound 21)

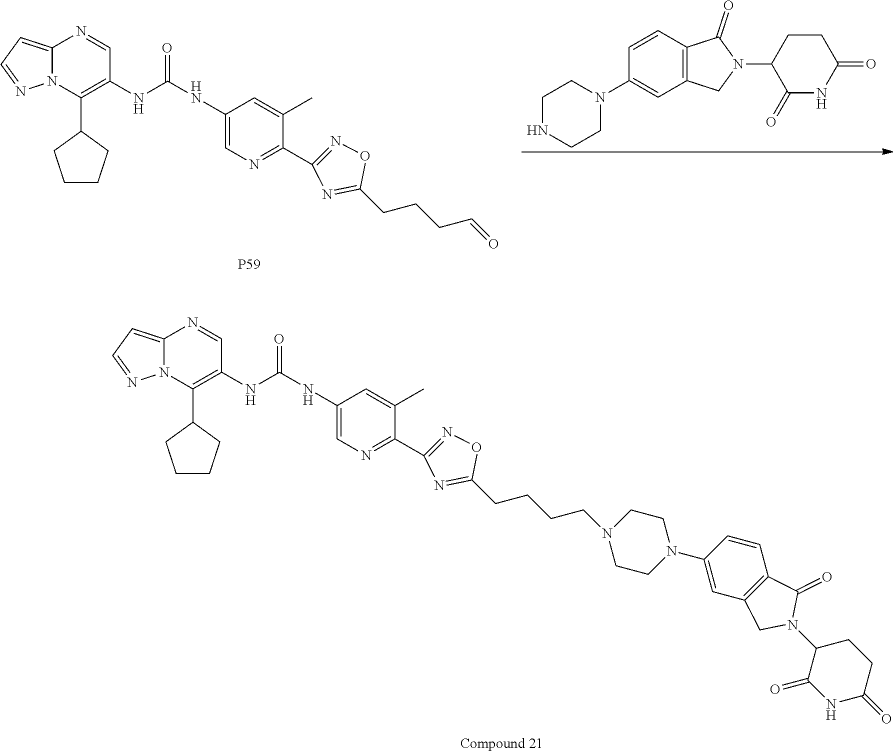

A solution of Compound P59 (0.1 g, 0.2 mmol), 3-(1-oxo-5-piperazin-1-yl-isoindolin-2-yl)piperidine-2,6-dione hydrochloride (0.08 g, 0.2 mmol) and potassium acetate (0.04 g, 0.4 mmol) in THF (6.0 mL) was stirred at ambient temperature for 1 h. Then, sodium triacetoxyborohydride (0.09 g, 0.4 mmol) was added and the mixture was stirred at ambient temperature for 15 h. The mixture was filtered through Celite and evaporated. The residue was purified by prep RP-HPLC on a C₁₈ column eluting with a gradient MeCN-H₂O+0.1% TFA. Yield of compound 21: 0.005 g, 3% on 2 steps (P59 and Compound 21). LCMS ESI (m/z): 787.8 ([M+1]⁺), 394.4, 339.0.

### Example 12. N-(7-Cyclopentylpyrazolo[1,5-a]pyrimidin-6-yl)-N′-{6-[5-(6-{4-[2-(2,6-dioxopiperidin-3-yl)-1-oxo-2,3-dihydro-1H-isoindol-5-yl]piperazin-1-yl}-6-oxohexyl)-1,2,4-oxadiazol-3-yl]-5-methylpyridin-3-yl}urea (Compound 4)

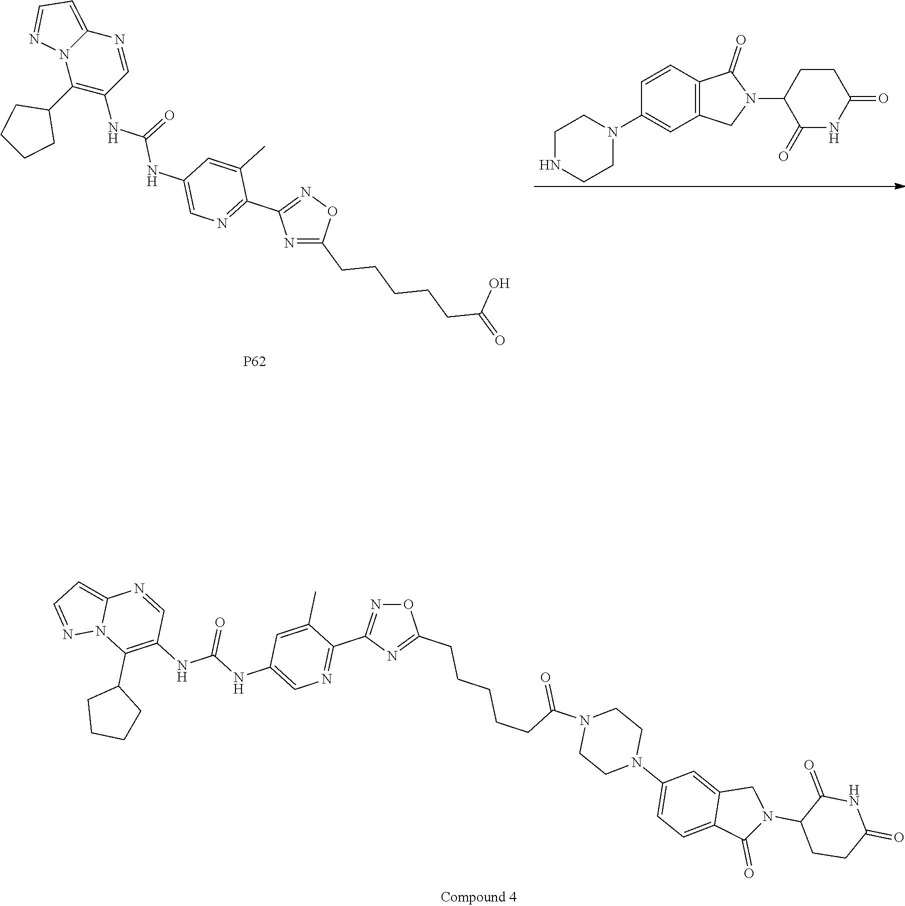 [SMILES: Cc1cc(NC(=O)Nc2cnc3ccnn3c2C2CCCC2)cnc1-c1noc(CCCCCC(=O)O)n1 ; "P62" ; O=C1CCC(N2Cc3cc(N4CCNCC4)ccc3C2=O)C(=O)N1 ; Cc1cc(NC(=O)Nc2cnc3ccnn3c2C2CCCC2)cnc1-c1noc(CCCCCC(=O)N2CCN(c3ccc4c(c3)CN(C3CCC(=O)NC3=O)C4=O)CC2)n1]

N,N-diisopropylpropan-2-amine (0.133 g, 0.93 mmol) was added to solution of 6-{3-[5-({[(7-cyclopentylpyrazolo[1,5-a]pyrimidin-6-yl)amino]carbonyl}amino)-3-methylpyridin-2-yl]-1,2,4-oxadiazol-5-yl}hexanoic acid P62 (0.096 g, 0.185 mmol) in DMF and the mixture was stirred for 5 min. 1-[Bis(dimethylamino)methylene]-1H-1,2,3-triazolo[4,5-b]pyridinium 3-oxide hexafluorophosphate (0.027 g, 0.072 mmol) was added and the mixture was stirred for 5 min. 3-(1-Oxo-5-piperazin-1-yl-1,3-dihydro-2H-isoindol-2-yl)piperidine-2,6-dione hydrochloride (0.067 g, 0.185 mmol) was added into the flask and stirred overnight at ambient temperature. The obtained crude residue was purified by RP-HPLC on a C18 column eluting with a gradient MeCN-H₂O+0.1% TFA to provide N-(7-cyclopentylpyrazolo[1,5-a]pyrimidin-6-yl)-N′-{6-[5-(6-{4-[2-(2,6-dioxopiperidin-3-yl)-1-oxo-2,3-dihydro-1H-isoindol-5-yl]piperazin-1-yl}-6-oxohexyl)-1,2,4-oxadiazol-3-yl]-5-methylpyridin-3-yl}urea—Compound 4 (0.05 g, 32%). ¹H NMR (400 MHz, DMSO-d₆), δ: 10.92 (s, 1H), 9.34 (s, 1H), 8.62 (s, 1H), 8.50 (s, 1H), 8.48 (s, 1H), 8.23 (d, J=2.1 Hz, 1H), 7.99 (s, 1H), 7.54 (d, J=8.3 Hz, 1H), 7.06 (d, J=8.9 Hz, 2H), 6.75 (d, J=2.1 Hz, 1H), 5.75 (s, 3H), 5.04 (dd, J=13.1, 5.1 Hz, 1H), 4.27 (dd, J=48.3, 16.8 Hz, 2H), 3.96-3.79 (m, 1H), 3.60 (s, 5H), 3.28-3.25 (m, 2H), 3.01 (t, J=7.4 Hz, 2H), 2.95-2.82 (m, 1H), 2.63-2.52 (m, 4H), 2.47 (s, 2H), 2.38 (t, J=7.2 Hz, 4H), 1.99 (s, 2H), 1.92-1.78 (m, 2H), 1.71 (s, 2H), 1.62-1.53 (m, 2H), 1.42 (d, J=7.5 Hz, 2H).

### Example 13. N-(7-Cyclopentylpyrazolo[1,5-a]pyrimidin-6-yl)-N′-(6-{1-[4-(2-{4-[2-(2,6-dioxopiperidin-3-yl)-1-oxo-2,3-dihydro-1H-isoindol-5-yl]piperazin-1-yl}-2-oxoethoxy)butyl]-1H-1,2,3-triazol-4-yl}-5-methylpyridin-3-yl)urea (Compound 8)

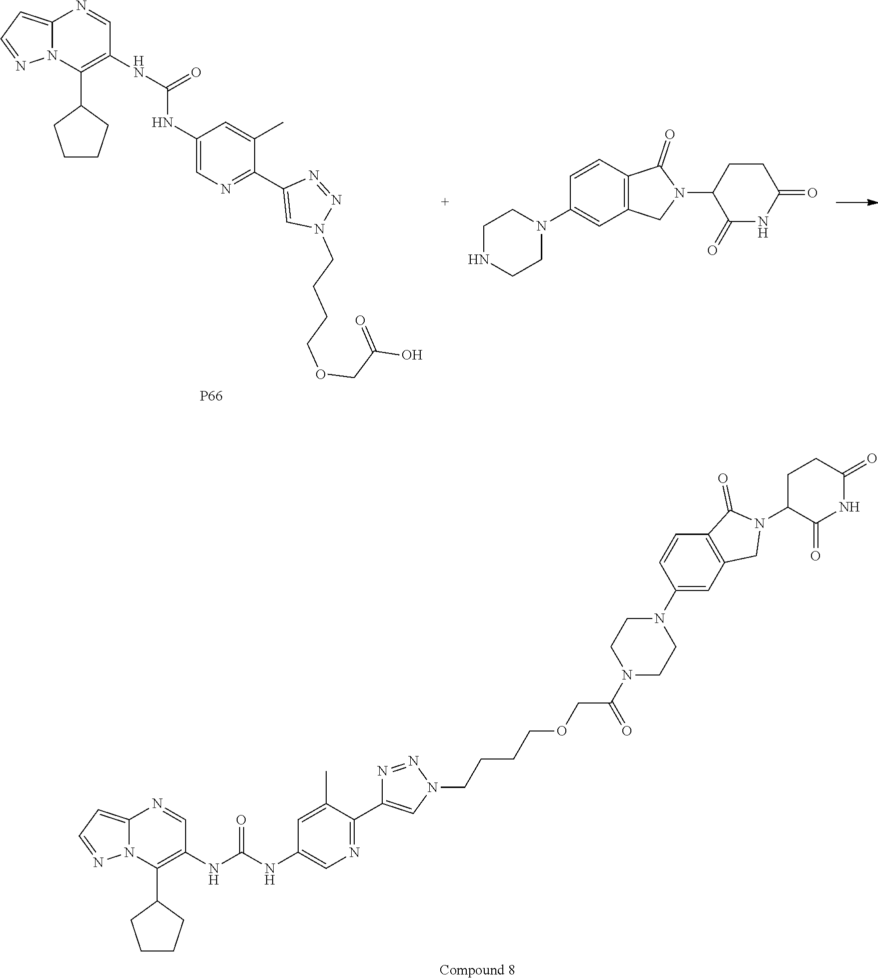 [SMILES: Cc1cc(NC(=O)Nc2cnc3ccnn3c2C2CCCC2)cnc1-c1cn(CCCCOCC(=O)O)nn1 ; "P66" ; Cc1cc(NC(=O)Nc2cnc3ccnn3c2C2CCCC2)cnc1-c1cn(CCCCOCC(=O)N2CCN(c3ccc4c(c3)CN(C3CCC(=O)NC3=O)C4=O)CC2)nn1 ; O=C1CCC(N2Cc3cc(N4CCNCC4)ccc3C2=O)C(=O)N1]

N-ethyl-N-isopropylpropan-2-amine (0.206 g, 1.56 mmol) was added to solution of (4-{4-[5-({[(7-cyclopentylpyrazolo[1,5-a]pyrimidin-6-yl)amino]carbonyl}amino)-3-methylpyridin-2-yl]-1H-1,2,3-triazol-l-yl}butoxy)acetic acid hydrochloride P66 (0.151 g, 0.27 mmol) in DMF and the mixture was stirred for 5 min. 1-[Bis(dimethylamino)methylene]-1H-1,2,3-triazolo[4,5-b]pyridinium 3-oxide hexafluorophosphate (0.111 g, 0.29 mmol) was added and the mixture was stirred for 5 min. 3-(1-oxo-5-piperazin-1-yl-1,3-dihydro-2H-isoindol-2-yl)piperidine-2,6-dione hydrochloride (0.097 g, 0.27 mmol) was added into the flask and stirred overnight at ambient temperature. The obtained crude residue was purified by RP-HPLC on a C18 column eluting with a gradient MeCN-H₂O+0.1% TFA to provide N-(7-cyclopentylpyrazolo[1,5-a]pyrimidin-6-yl)-N′-(6-{1-[4-(2-{4-[2-(2,6-dioxopiperidin-3-yl)-1-oxo-2,3-dihydro-1H-isoindol-5-yl]piperazin-1-yl}-2-oxoethoxy)butyl]-1H-1,2,3-triazol-4-yl}-5-methylpyridin-3-yl)urea—Compound 8 (0.016 g, 7%). ¹H NMR (400 MHz, DMSO-d₆), δ: 10.93 (s, 1H), 9.48 (s, 1H), 8.83-8.56 (m, 2H), 8.47 (s, 1H), 8.23 (d, J=2.3 Hz, 1H), 8.02 (s, 1H), 7.53 (d, J=8.4 Hz, 1H), 7.22 (s, 1H), 7.07 (t, J=8.9 Hz, 2H), 6.75 (d, J=2.3 Hz, 1H), 5.04 (dd, J=13.2, 5.0 Hz, 1H), 4.51 (t, J=6.9 Hz, 1H), 4.33 (d, J=17.0 Hz, 2H), 4.20 (d, J=19.4 Hz, 4H), 3.88 (d, J=9.0 Hz, 2H), 3.57 (s, 4H), 3.49 (t, J=6.2 Hz, 3H), 3.30 (m, 4H), 2.87 (d, J=12.7 Hz, 2H), 2.75-2.56 (m, 2H), 2.36 (d, J=8.6 Hz, 2H), 2.02 (d, J=37.3 Hz, 4H), 1.88 (d, J=18.1 Hz, 2H), 1.70 (s, 2H), 1.55 (d, J=7.1 Hz, 2H).

### Example 14. N-(7-cyclopentylpyrazolo[1,5-a]pyrimidin-6-yl)-N′-(6-{5-[(4-{4-[2-(2,6-dioxopiperidin-3-yl)-1-oxo-2,3-dihydro-1H-isoindol-5-yl]piperazin-1-yl}butoxy)methyl]-1,2,4-oxadiazol-3-yl}-5-methylpyridin-3-yl)urea—Compound 11

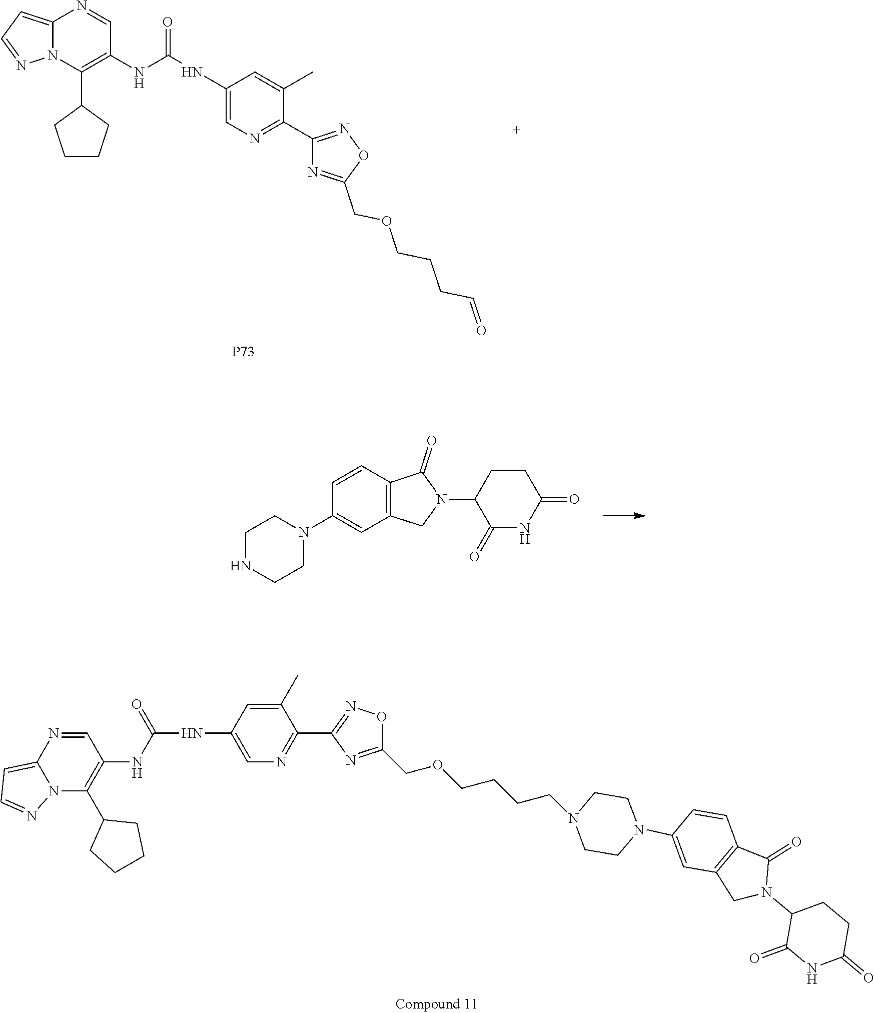 [SMILES: Cc1cc(NC(=O)Nc2cnc3ccnn3c2C2CCCC2)cnc1-c1noc(COCCCC=O)n1 ; "P73" ; O=C1CCC(N2Cc3cc(N4CCNCC4)ccc3C2=O)C(=O)N1 ; Cc1cc(NC(=O)Nc2cnc3ccnn3c2C2CCCC2)cnc1-c1noc(COCCCCN2CCN(c3ccc4c(c3)CN(C3CCC(=O)NC3=O)C4=O)CC2)n1]

A solution of Compound P73 (0.077 g, 0.15 mmol), 3-(1-oxo-5-piperazin-1-yl-1,3-dihydro-2H-isoindol-2-yl)piperidine-2,6-dione hydrochloride (0.056 g, 0.15 mmol) and potassium acetate (0.03 g, 0.30 mmol) in THF (6.0 mL) was stirred at ambient temperature for 1 h. Then, sodium triacetoxyborohydride (0.07 g, 0.30 mmol) was added and the mixture was stirred at ambient temperature for 15 h. The mixture was filtered through Celite and evaporated. The residue was purified by prep RP-HPLC on a C18 column eluting with a gradient MeCN-H₂O+0.1% TFA. Yield of compound 11: 0.015 g, 12% (on 2 steps: P73, Compound 11). LCMS ESI (m/z): 817.5 ([M+1]⁺), 409.5.

### Example 15. N-(7-cyclopentylpyrazolo[1,5-a]pyrimidin-6-yl)-N′-(6-{5-[2-(2-{4-[2-(2,6-dioxopiperidin-3-yl)-1-oxo-2,3-dihydro-1H-isoindol-5-yl]piperazin-1-yl}ethoxy)ethyl]-1,2,4-oxadiazol-3-yl}-5-methylpyridin-3-yl)urea (Compound 16)

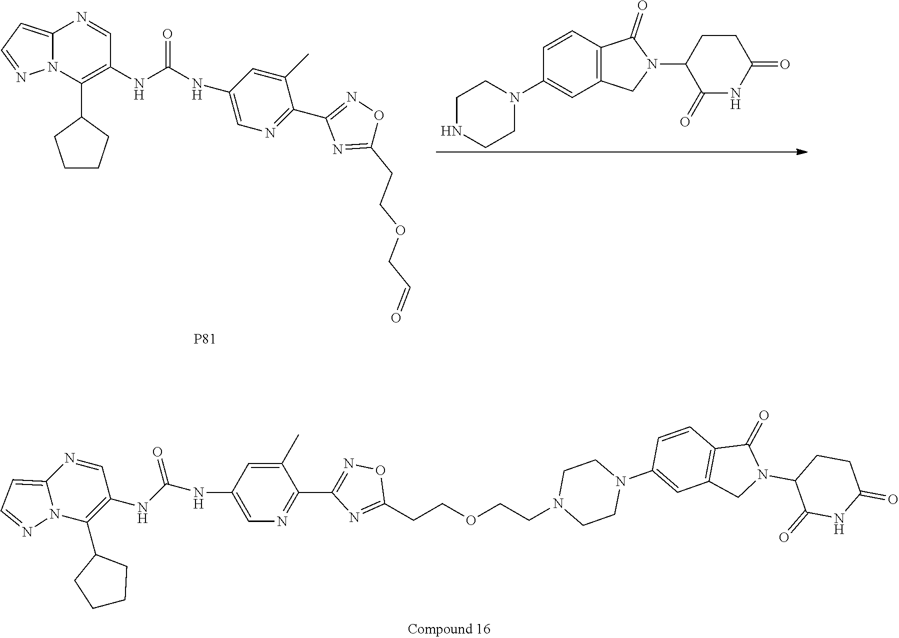 [SMILES: "P81" ; Cc1cc(NC(=O)Nc2cnc3ccnn3c2C2CCCC2)cnc1-c1noc(CCOCCN2CCN(c3ccc4c(c3)CN(C3CCC(=O)NC3=O)C4=O)CC2)n1 ; Cc1cc(NC(=O)Nc2cnc3ccnn3c2C2CCCC2)cnc1-c1noc(CCOCC=O)n1 ; O=C1CCC(N2Cc3cc(N4CCNCC4)ccc3C2=O)C(=O)N1]

A solution of Compound P81 (0.18 g, 0.36 mmol), 3-(1-oxo-5-piperazin-1-yl-1,3-dihydro-2H-isoindol-2-yl)piperidine-2,6-dione hydrochloride (0.13 g, 0.36 mmol) and potassium acetate (0.07 g, 0.72 mmol) in THF (6.0 mL) was stirred at ambient temperature for 1 h. Then, sodium triacetoxyborohydride (0.16 g, 0.72 mmol) was added and the mixture was stirred at ambient temperature for 15 h. The mixture was filtered through Celite and evaporated. The residue was purified by prep RP-HPLC on a C18 column eluting with a gradient MeCN-H₂O+0.1% TFA. Yield of compound 16: 0.01 g, 4% on 2 steps (P81 and Compound 16).

### Example 16. N-(7-cyclopentylpyrazolo[1,5-a]pyrimidin-6-yl)-N′-{6-[5-(3-{4-[2-(2,6-dioxopiperidin-3-yl)-6-fluoro-1,3-dioxo-2,3-dihydro-1H-isoindol-5-yl]piperazin-1-yl}propyl)-1,2,4-oxadiazol-3-yl]-5-methylpyridin-3-yl}urea (Compound 23)

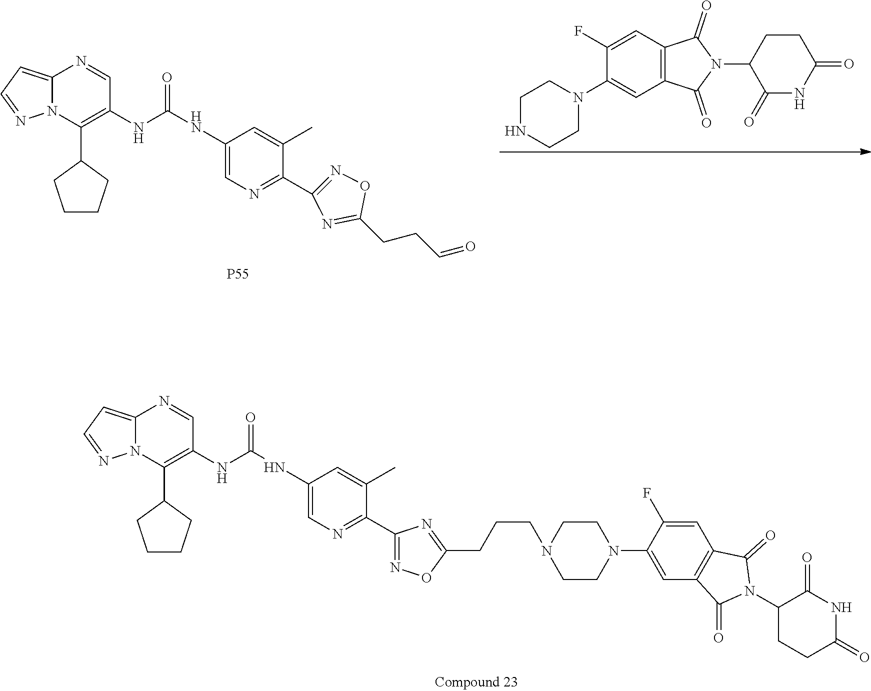

A solution of Compound P55 (0.10 g, 0.22 mmol), 2-(2,6-dioxo-3-piperidyl)-5-fluoro-6-piperazin-1-yl-isoindoline-1,3-dione hydrochloride (0.09 g, 0.22 mmol) and potassium acetate (0.04 g, 0.44 mmol) in THF (6.0 mL) was stirred at ambient temperature for 1 h. Then, sodium triacetoxyborohydride (0.10 g, 0.44 mmol) was added and the mixture was stirred at ambient temperature for additional 15 h. The mixture was filtered through Celite and evaporated. The residue was purified by prep RP-HPLC on a C18 column eluting with a gradient MeCN-H₂O+0.1% TFA. Yield of compound 23: 0.04 g, 23% on 2 steps (P55 and Compound 23). ¹H NMR (400 MHz, DMSO-d₆), δ: 11.11 (s, 1H), 9.74 (s, 1H), 9.50 (s, 1H), 8.76-8.55 (m, 2H), 8.47 (s, 1H), 8.23 (d, J=2.2 Hz, 1H), 7.99 (s, 1H), 7.82 (d, J=11.1 Hz, 1H), 7.61 (d, J=7.4 Hz, 1H), 6.75 (d, J=2.2 Hz, 1H), 5.12 (dd, J=12.9, 5.3 Hz, 1H), 3.85 (dd, J=18.3, 9.0 Hz, 4H), 3.65 (s, 2H), 3.31 (d, J=22.9 Hz, 6H), 3.16 (t, J=7.1 Hz, 2H), 2.89 (t, J=12.9 Hz, 1H), 2.70-2.55 (m, 2H), 2.33 (s, 2H), 2.27 (dd, J=15.5, 7.8 Hz, 2H), 2.07-1.94 (m, 4H), 1.88 (d, J=17.7 Hz, 2H), 1.71 (s, 2H).

### Example 17. N-(3-chloro-7-cyclopentylpyrazolo[1,5-a]pyrimidin-6-yl)-N′-{6-[5-(3-{4-[2-(2,6-dioxopiperidin-3-yl)-6-fluoro-1,3-dioxo-2,3-dihydro-1H-isoindol-5-yl]piperazin-1-yl}propyl)-1,2,4-oxadiazol-3-yl]-5-methylpyridin-3-yl}urea (Compound 24)

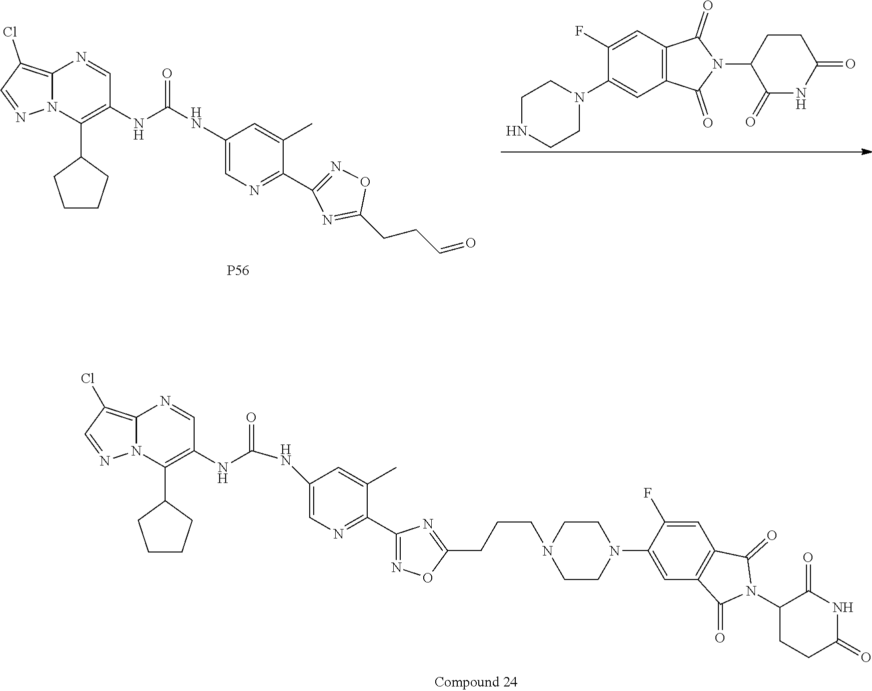

A solution of Compound P56 (0.10 g, 0.22 mmol), 2-(2,6-dioxo-3-piperidyl)-5-fluoro-6-piperazin-1-yl-isoindoline-1,3-dione hydrochloride (0.09 g, 0.22 mmol) and potassium acetate (0.04 g, 0.44 mmol) in THF (6.0 mL) was stirred at ambient temperature for 1 h. Then, sodium triacetoxyborohydride (0.10 g, 0.44 mmol) was added and the mixture was stirred at ambient temperature for 15 h. The mixture was filtered through Celite and evaporated. The residue was purified by prep RP-HPLC on a C18 column eluting with a gradient MeCN-H₂O+0.1% TFA. Yield of compound 24: 0.044 g, 25% on 2 steps (P56 and Compound 24). ¹H NMR (400 MHz, DMSO-d₆), δ: 11.11 (s, 1H), 9.75 (s, 1H), 9.51 (s, 1H), 8.78 (s, 1H), 8.65 (s, 1H), 8.59 (s, 1H), 8.40 (s, 1H), 7.99 (s, 1H), 7.82 (d, J=11.1 Hz, 1H), 7.61 (d, J=7.3 Hz, 1H), 5.12 (dd, J=12.9, 5.3 Hz, 1H), 3.85 (dd, J=18.4, 8.9 Hz, 4H), 3.65 (s, 2H), 3.31 (d, J=21.8 Hz, 6H), 3.16 (t, J=7.3 Hz, 2H), 2.89 (t, J=12.9 Hz, 1H), 2.60 (dd, J=32.7, 14.9 Hz, 2H), 2.37-2.16 (m, 4H), 2.02 (d, J=37.4 Hz, 4H), 1.88 (s, 2H), 1.70 (s, 2H).

### Example 18. N-(5-Cyclopentylimidazo[1,2-a]pyrimidin-6-yl)-N′-{6-[5-(6-{4-[2-(2,6-dioxopiperidin-3-yl)-1-oxo-2,3-dihydro-1H-isoindol-5-yl]piperazin-1-yl}-6-oxohexyl)-1,2,4-oxadiazol-3-yl]-5-methylpyridin-3-yl}urea (Compound 9)

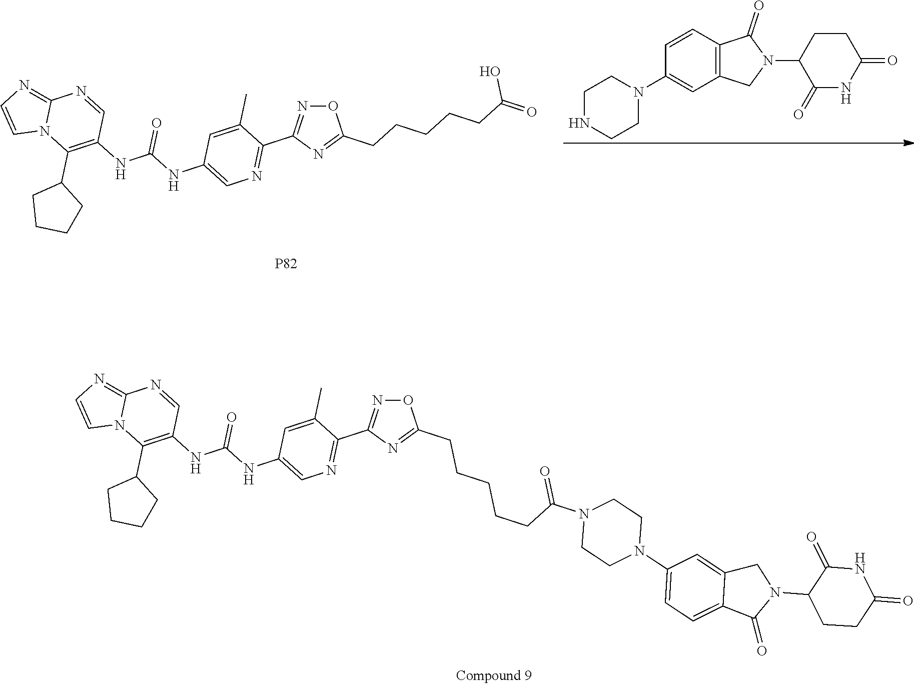 [SMILES: Cc1cc(NC(=O)Nc2cnc3nccn3c2C2CCCC2)cnc1-c1noc(CCCCCC(=O)O)n1 ; "P82" ; Cc1cc(NC(=O)Nc2cnc3nccn3c2C2CCCC2)cnc1-c1noc(CCCCCC(=O)N2CCN(c3ccc4c(c3)CN(C3CCC(=O)NC3=O)C4=O)CC2)n1 ; O=C1CCC(N2Cc3cc(N4CCNCC4)ccc3C2=O)C(=O)N1]

N,N-Diisopropylpropan-2-amine (0.142 g, 1.09 mmol) was added to solution of Compound P82 (0.095 g, 0.183 mmol) in DMF and the mixture stirred for 5 min. 1-[bis(Dimethylamino)methylene]-1H-1,2,3-triazolo[4,5-b]pyridinium 3-oxide hexafluorophosphate (0.076 g, 0.201 mmol) was added and the mixture was stirred for 5 min. 3-(1-Oxo-5-piperazin-1-yl-1,3-dihydro-2H-isoindol-2-yl)piperidine-2,6-dione hydrochloride (0.067 g, 0.185 mmol) was added into the flask and stirred overnight at ambient temperature. The obtained crude residue was purified by RP-HPLC on a C18 column eluting with a gradient MeCN-H₂O+0.1% TFA to provide Compound 9 (0.04 g, 26%). ¹H NMR (400 MHz, DMSO-d₆), δ: 10.92 (s, 1H), 9.75 (s, 1H), 9.11 (s, 1H), 8.97 (s, 1H), 8.66 (s, 1H), 8.27 (d, J=12.4 Hz, 2H), 7.97 (s, 1H), 7.53 (d, J=8.6 Hz, 1H), 7.34 (t, J=8.0 Hz, 1H), 7.17 (d, J=8.4 Hz, 1H), 7.06 (d, J=9.0 Hz, 3H), 5.04 (dd, J=13.2, 5.0 Hz, 2H), 4.27 (dd, J=48.5, 17.0 Hz, 2H), 3.95-3.86 (m, 2H), 3.60 (s, 4H), 3.29 (d, J=21.9 Hz, 4H), 3.01 (t, J=7.2 Hz, 2H), 2.97-2.83 (m, 2H), 2.57 (d, J=25.3 Hz, 4H), 2.38 (t, J=7.4 Hz, 2H), 2.23-1.91 (m, 4H), 1.80 (dd, J=20.5, 12.6 Hz, 2H), 1.67-1.50 (m, 2H), 1.42 (d, J=7.1 Hz, 2H).

### Example 19. N-(7-Cyclopentylpyrazolo[1,5-a]pyrimidin-6-yl)-N′-{6-[5-(4-{4-[2-(2,6-dioxopiperidin-3-yl)-1-oxo-2,3-dihydro-1H-isoindol-5-yl]piperazin-1-yl}-2,2-dimethyl-4-oxobutyl)-1,2,4-oxadiazol-3-yl]-5-methylpyridin-3-yl}urea (Compound 10)

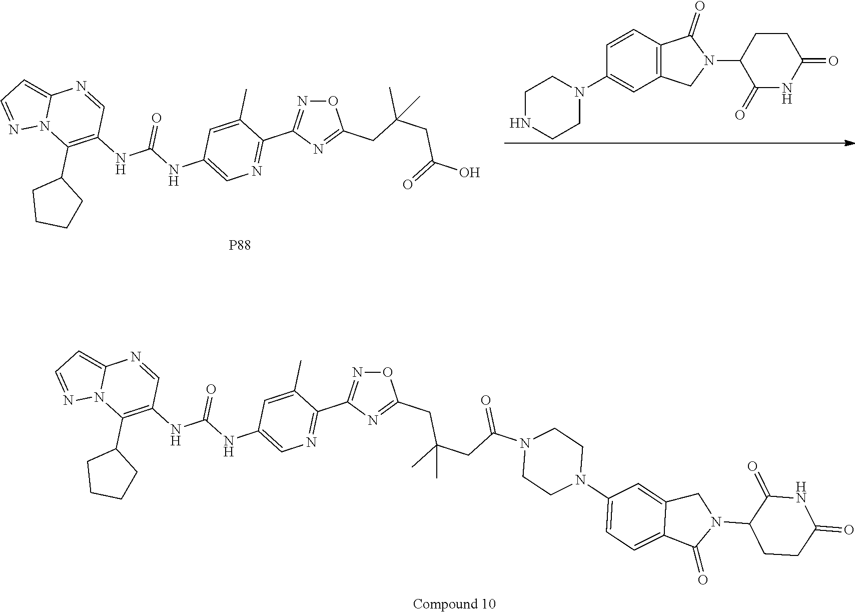 [SMILES: "P88" ; Cc1cc(NC(=O)Nc2cnc3ccnn3c2C2CCCC2)cnc1-c1noc(CC(C)(C)CC(=O)O)n1 ; Cc1cc(NC(=O)Nc2cnc3ccnn3c2C2CCCC2)cnc1-c1noc(CC(C)(C)CC(=O)N2CCN(c3ccc4c(c3)CN(C3CCC(=O)NC3=O)C4=O)CC2)n1 ; O=C1CCC(N2Cc3cc(N4CCNCC4)ccc3C2=O)C(=O)N1]

N-Ethyl-N-isopropylpropan-2-amine (58.2 mg, 0.45 mmol) was added to solution of Compound P88 (39 mg, 0.075 mmol) in DMF and the mixture stirred for 5 min. 1-[bis(Dimethylamino)methylene]-1H-1,2,3-triazolo[4,5-b]pyridinium 3-oxide hexafluorophosphate (31.4 mg, 0.083 mmol) was added and the mixture was stirred for 5 min. 3-(1-Oxo-5-piperazin-1-yl-1,3-dihydro-2H-isoindol-2-yl)piperidine-2,6-dione hydrochloride (0.067 g, 0.185 mmol) was added into the flask and stirred overnight at ambient temperature. The obtained crude residue was purified by RP-HPLC on a C18 column eluting with a gradient MeCN-H₂O+0.1% TFA to provide Compound 10 (8.7 mg, 14%). H NMR (400 MHz, DMSO-d₆), δ: 10.92 (s, 1H), 9.35 (s, 1H), 8.61 (s, 1H), 8.49 (d, J=11.3 Hz, 2H), 8.22 (s, 1H), 7.99 (s, 1H), 7.54 (d, J=8.4 Hz, 1H), 7.06 (d, J=9.1 Hz, 2H), 6.74 (s, 1H), 5.04 (d, J=8.8 Hz, 1H), 4.28 (dd, J=48.1, 16.9 Hz, 2H), 3.98-3.80 (m, 1H), 3.68 (m, 2H), 3.66 (s, 3H), 2.91 (t, J=22.8 Hz, 2H), 2.55 (t, J=15.2 Hz, 8H), 2.35 (s, 4H), 1.98 (s, 2H), 1.86 (s, 2H), 1.71 (s, 2H), 1.02 (m, 2H), 1.01 (s, 6H).

### Example 20. N-(7-Cyclopentylpyrazolo[1,5-a]pyrimidin-6-yl)-N′-{6-[5-(5-{4-[2-(2,6-dioxopiperidin-3-yl)-1-oxo-2,3-dihydro-1H-isoindol-5-yl]piperazin-1-yl}-5-oxopentyl)-1,2,4-oxadiazol-3-yl]-5-methylpyridin-3-yl}urea (Compound 14)

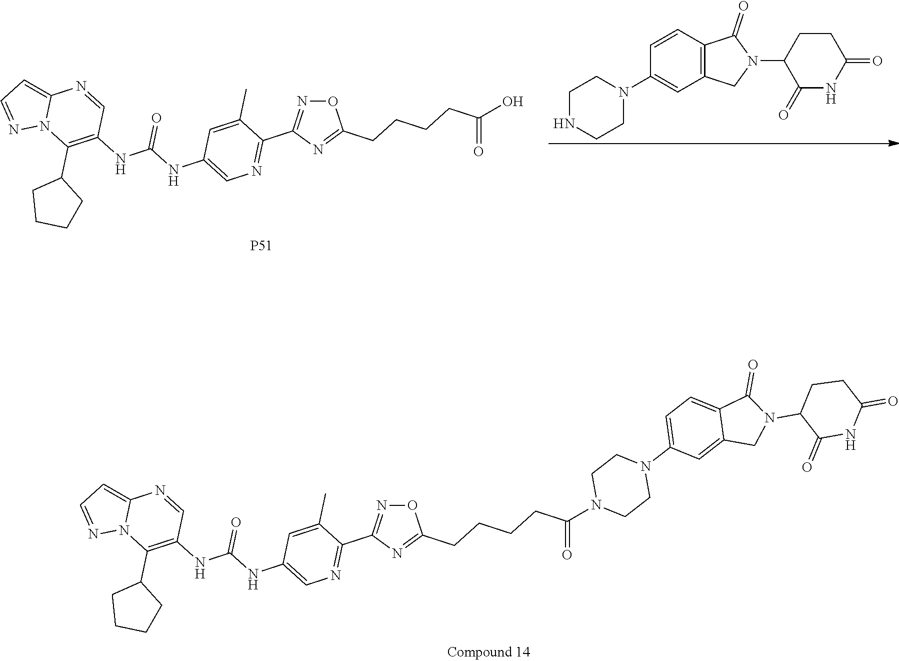

DIPEA (0.216 g, 1.67 mmol) was added to a solution of Compound P51 (0.141 g, 0.278 mmol) in DMF and the mixture stirred for 5 min. N-[(dimethylamino)(3-oxido-1H-[1,2,3]triazolo[4,5-b]pyridin-1-yl)methylene]-N-methylmethanaminium hexafluorophosphate (0.117 g, 0.306 mmol) was added and the mixture was stirred for 5 min. 3-(1-Oxo-5-piperazin-1-yl-1,3-dihydro-2H-isoindol-2-yl)piperidine-2,6-dione hydrochloride (0.102 g, 0.279 mmol) was added into the flask and stirred overnight at ambient temperature. The obtained crude residue was purified by RP-HPLC on a C18 column eluting with a gradient MeCN-H₂O+0.1% TFA to provide Compound 14 (0.077 g, 33%). ¹H NMR (400 MHz, DMSO-d₆), δ: 10.94 (s, 1H), 9.36 (s, 1H), 8.62 (d, J=2.0 Hz, 1H), 8.52 (s, 1H), 8.48 (s, 1H), 8.23 (d, J=2.3 Hz, 1H), 7.99 (d, J=1.9 Hz, 1H), 7.54 (d, J=8.4 Hz, 1H), 7.06 (d, J=9.7 Hz, 2H), 6.75 (d, J=2.3 Hz, 1H), 5.04 (dd, J=13.2, 5.0 Hz, 1H), 4.27 (dd, J=49.1, 17.1 Hz, 2H), 3.86 (t, J=9.0 Hz, 1H), 3.61 (s, 4H), 3.28 (s, 2H), 3.04 (t, J=7.3 Hz, 2H), 2.89 (t, J=12.8 Hz, 1H), 2.63 (d, J=28.5 Hz, 1H), 2.54 (s, 3H), 2.47-2.42 (m, 2H), 2.36 (d, J=8.5 Hz, 2H), 1.98 (s, 2H), 1.92-1.79 (m, 4H), 1.78-1.56 (m, 4H).

### Example 21. N-(3-Chloro-7-cyclopentylpyrazolo[1,5-a]pyrimidin-6-yl)-N′-{5-chloro-6-[5-(4-{4-[2-(2,6-dioxopiperidin-3-yl)-1-oxo-2,3-dihydro-1H-isoindol-5-yl]piperazin-1-yl}butyl)-1,2,4-oxadiazol-3-yl]pyridin-3-yl}urea (Compound 22)

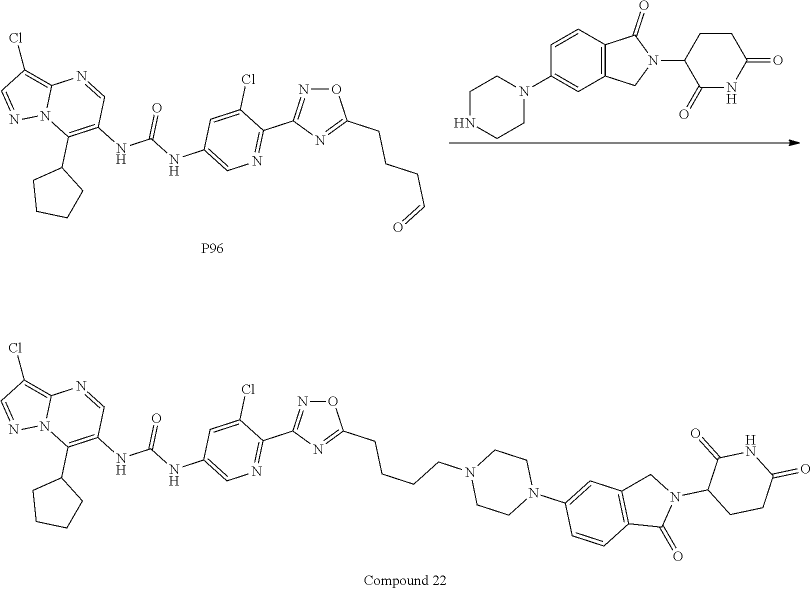

A solution of Compound P96 (23.8 mg, 0.048 mmol), 3-(1-oxo-5-piperazin-1-yl-1,3-dihydro-2H-isoindol-2-yl)piperidine-2,6-dione hydrochloride (17.5 mg, 0.048 mmol) and potassium acetate (9.4 mg, 0.096 mmol) in THF (6.0 ml) was stirred at ambient temperature for 1 h. After this, sodium triacetoxyborohydride (20.4 mg, 0.096 mmol) was added and the mixture was stirred at ambient temperature for 15 h. The mixture was filtered through Celite and evaporated. The residue was purified by prep RP-HPLC on a C18 column eluting with a gradient MeCN-H₂O+0.1% TFA. Yield of the Compound 22 on 2 steps: 0.0117 g, 29%. ¹H NMR (400 MHz, DMSO-d₆), δ: 10.96 (s, 1H), 9.61 (s, 1H), 9.04 (s, 1H), 8.73 (d, J=2.1 Hz, 1H), 8.59 (s, 1H), 8.41 (s, 1H), 8.35 (d, J=2.1 Hz, 1H), 7.59 (d, J=8.4 Hz, 1H), 7.24 (s, 1H), 7.21-7.12 (m, 2H), 7.11 (s, 1H), 6.98 (s, 1H), 5.06 (dd, J=13.0, 4.9 Hz, 1H), 4.29 (dd, J=49.0, 17.0 Hz, 2H), 3.86 (d, J=9.5 Hz, 2H), 3.60 (t, J=6.3 Hz, 6H), 3.24 (s, 2H), 3.12 (d, J=7.1 Hz, 2H), 2.70 (d, J=17.8 Hz, 2H), 2.34 (d, J=13.3 Hz, 2H), 1.97 (s, 2H), 1.88 (d, J=18.8 Hz, 2H), 1.80-1.73 (m, 4H), 1.70 (s, 2H).

### Example 22. 3-(2-{4-[2-(2,6-Dioxopiperidin-3-yl)-1-oxo-2,3-dihydro-1H-isoindol-5-yl]piperazin-1-yl}ethoxy)-N-[(3-{3-methyl-5-[({[7-(3,4,5-trimethoxyphenyl)pyrazolo[1,5-a]pyrimidin-6-yl]amino}carbonyl)amino]pyridin-2-yl}-1,2,4-oxadiazol-5-yl)methyl]propenamide—Compound 1

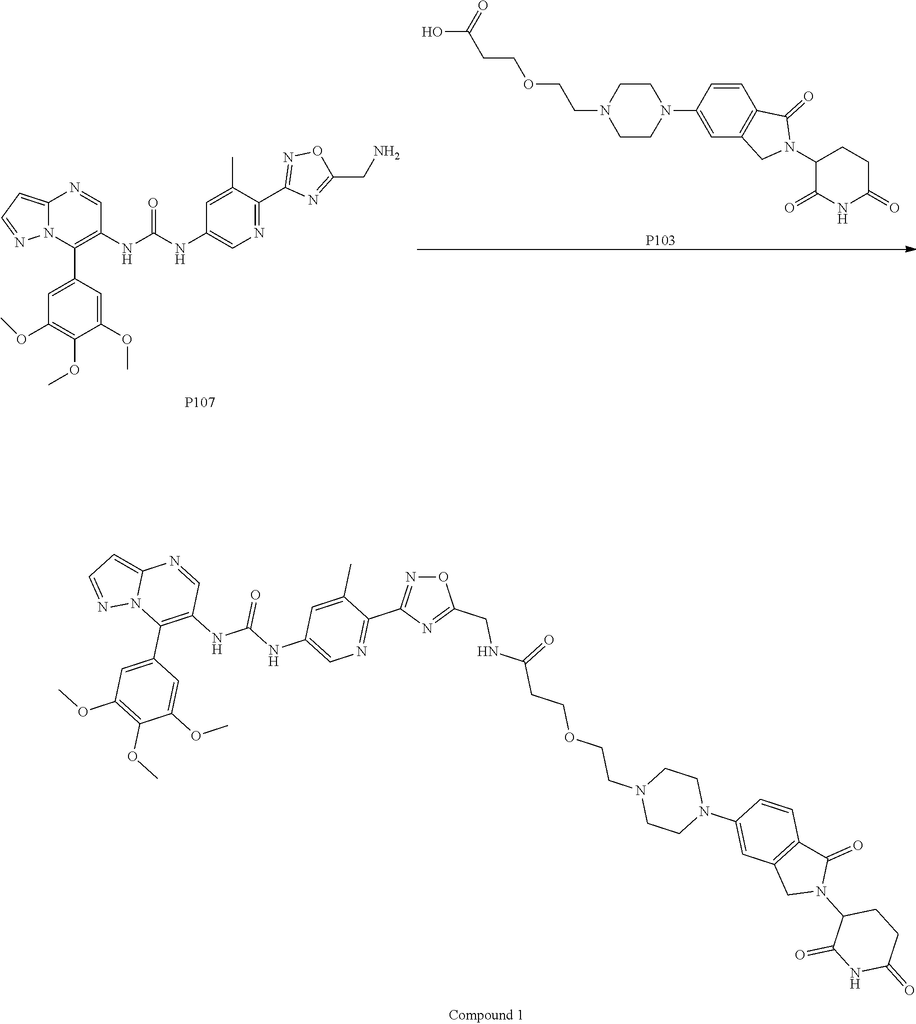 [SMILES: "P107" ; COc1cc(-c2c(NC(=O)Nc3cnc(-c4noc(CN)n4)c(C)c3)cnc3ccnn23)cc(OC)c1OC ; COc1cc(-c2c(NC(=O)Nc3cnc(-c4noc(CNC(=O)CCOCCN5CCN(c6ccc7c(c6)CN(C6CCC(=O)NC6=O)C7=O)CC5)n4)c(C)c3)cnc3ccnn23)cc(OC)c1OC ; "P103" ; O=C(O)CCOCCN1CCN(c2ccc3c(c2)CN(C2CCC(=O)NC2=O)C3=O)CC1]

To a solution of Compound P107 (53.0 mg, 0.10 mmol), Compound P103 (44 mg, 0.10 mmol) and HATU (57.0 mg, 0.15 mmol) in DMF (1 ml) a DIPEA (68 mg, 0.53 mmol) was added, and mixture was stirred at ambient temperature for 15 h. Then solution was diluted with water (5 ml), the solid was filtered off and dried. The residue was purified by prep RP-HPLC on a C18 column eluting with a gradient MeCN-H₂O+0.1% TFA giving Compound 1: 20 mg, 24%. ¹H NMR (400 MHz, DMSO-d₆), δ: 10.93 (s, 1H), 9.40 (s, 1H), 8.83 (s, 2H), 8.58 (s, 1H), 8.19 (d, J=22.6 Hz, 2H), 7.90 (s, 1H), 7.49 (s, 1H), 7.02 (d, J=18.5 Hz, 4H), 6.82 (s, 1H), 5.03 (s, 1H), 4.64 (s, 2H), 4.25 (d, J=30.7 Hz, 2H), 3.74 (s, 9H), 3.61 (d, J=32.8 Hz, 6H), 3.28-3.12 (m, 6H), 2.89 (s, 2H), 2.30 (s, 3H), 2.39-2.26 (m, 2H), 1.95 (s, 2H), 1.24 (s, 2H).

### Example 23. N-(7-Cyclopentylpyrazolo[1,5-a]pyrimidin-6-yl)-N′-(6-{1-[2-(2-{2-[({[2-(2,6-dioxopiperidin-3-yl)-1-oxo-2,3-dihydro-1H-isoindol-4-yl]amino}carbonyl)amino]ethoxy}ethoxy)ethyl]-1H-1,2,3-triazol-4-yl}-5-methylpyridin-3-yl)urea (Compound 7)

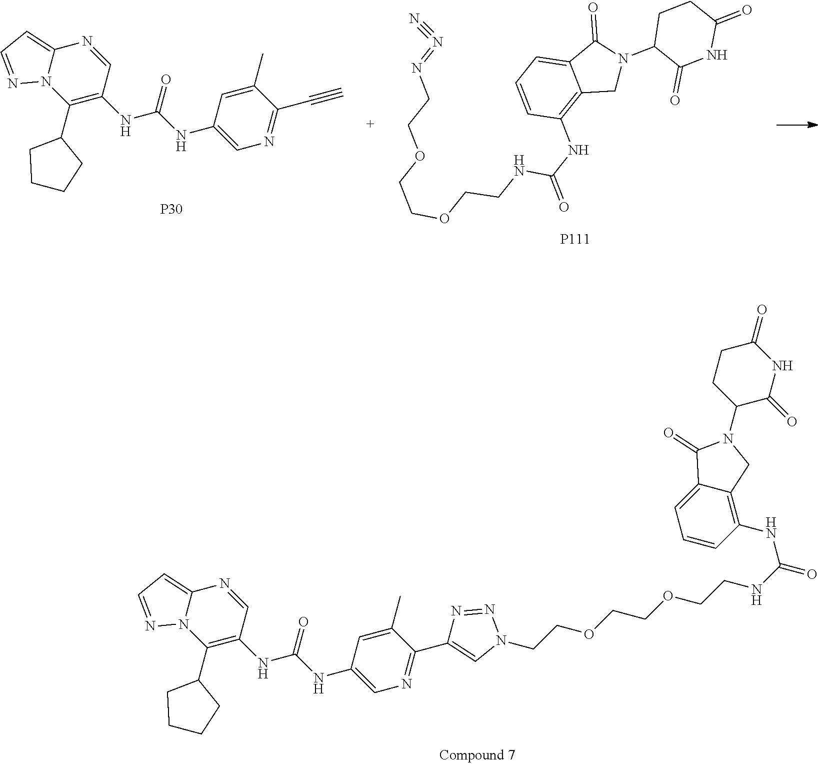

N-(7-Cyclopentylpyrazolo[1,5-a]pyrimidin-6-yl)-N-(6-ethynyl-5-methylpyridin-3-yl)urea P30 (0.18 g, 0.50 mmol), and N-[2-(2,6-dioxopiperidin-3-yl)-1-oxo-2,3-dihydro-1H-isoindol-4-yl]-N′-(2-{2-[2-(2λ⁵-triaz-1-en-2-yn-1-yl)ethoxy]ethoxy}ethyl)urea P111 (0.23 g, 0.50 mmol) were dissolved in 20 mL of a solvent consisting of tetrahydrofuran and water in equal amounts. Copper(II) acetate (0.009 g, 0.05 mmol), and sodium ascorbate (0.010 g, 0.05 mmol) were added into the flask and the mixture was stirred for 12 h at rt. After completion of the reaction as indicated by LCMS, the product was extracted with ethyl acetate and concentrated. The obtained crude residue was purified by RP-HPLC on a C18 column eluting with a gradient MeCN-H₂O+0.1% TFA to provide N-(7-Cyclopentylpyrazolo[1,5-a]pyrimidin-6-yl)-N′-(6-{1-[2-(2-{2-[({[2-(2,6-dioxopiperidin-3-yl)-1-oxo-2,3-dihydro-1H-isoindol-4-yl]amino}carbonyl)amino]ethoxy}ethoxy)ethyl]-1H-1,2,3-triazol-4-yl}-5-methylpyridin-3-yl)urea—Compound 7 (0.0032 g, 8%). LCMS ESI (m/z): 820.3 ([M+1]⁺), 535.8, 429.4.

### Example 24. N-(7-Cyclopentylpyrazolo[1,5-a]pyrimidin-6-yl)-N′-{6-[5-({3-[(5-{4-[2-(2,6-dioxopiperidin-3-yl)-1-oxo-2,3-dihydro-1H-isoindol-5-yl]piperazin-1-yl}pentyl)oxy]propoxy}methyl)-1,2,4-oxadiazol-3-yl]-5-methylpyridin-3-yl}urea (Compound 15)

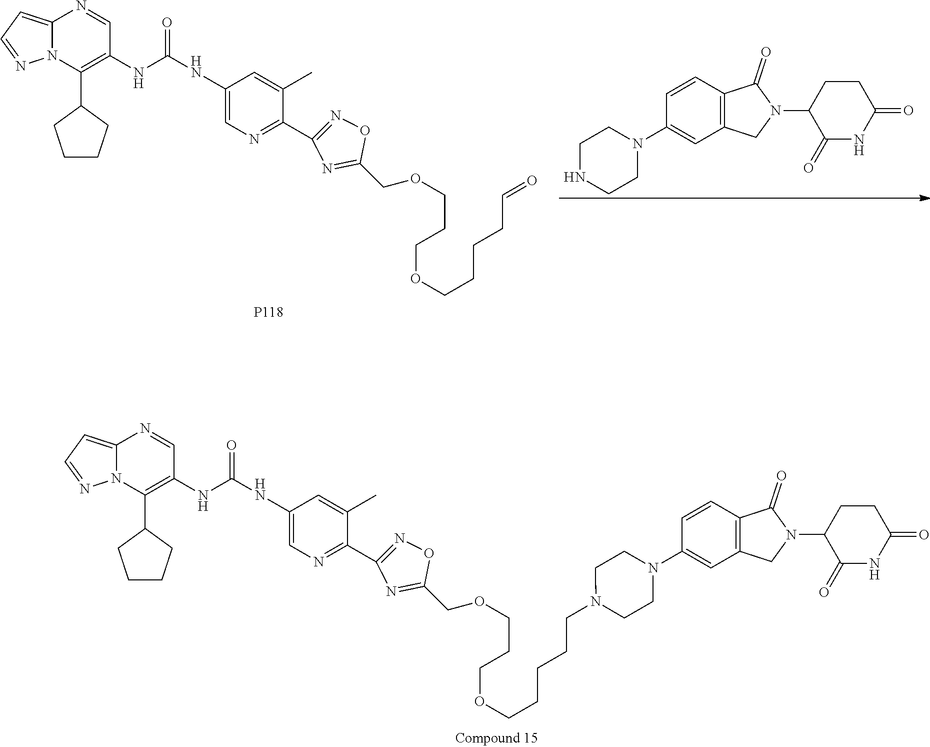

A solution of Compound P118 (0.108 g, 0.188 mmol), 3-(1-oxo-5-piperazin-1-yl-1,3-dihydro-2H-isoindol-2-yl)piperidine-2,6-dione hydrochloride (0.069 g, 0.188 mmol) and potassium acetate (0.037 g, 0.376 mmol) in THF (6.0 ml) was stirred at ambient temperature for 1 h. Then, sodium triacetoxyborohydride (0.08 g, 0.376 mmol) was added and the mixture was stirred at ambient temperature for 15 h. The mixture was filtered through Celite and evaporated. The residue was purified by prep RP-HPLC on a C18 column eluting with a gradient MeCN-H₂O+0.1% TFA. The yield of compound 15 on 2 steps (0.011 g) was 6%. ¹H NMR (400 MHz, DMSO-d₆), δ: 10.95 (s, 1H), 9.54 (s, 1H), 8.70 (s, 1H), 8.66 (s, 1H), 8.48 (s, 1H), 8.23 (d, J=2.3 Hz, 1H), 8.00 (s, 1H), 7.58 (d, J=8.4 Hz, 1H), 7.15 (d, J=5.4 Hz, 1H), 6.75 (d, J=2.3 Hz, 1H), 5.06 (dd, J=13.1, 4.9 Hz, 1H), 4.88 (s, 1H), 4.28 (dd, J=47.5, 17.2 Hz, 2H), 4.01 (d, J=10.0 Hz, 1H), 3.92-3.81 (m, 1H), 3.65 (t, J=6.4 Hz, 2H), 3.57 (s, 1H), 3.44 (t, J=6.3 Hz, 2H), 3.37 (t, J=6.3 Hz, 4H), 3.12 (s, 4H), 2.90 (t, J=12.8 Hz, 2H), 2.69-2.56 (m, 2H), 2.42-2.25 (m, 4H), 2.30 (s, 3H), 1.98 (s, 2H), 1.85 (d, J=11.2 Hz, 4H), 1.83-1.77 (m, 4H), 1.69 (s, 2H), 1.52 (d, J=7.6 Hz, 2H), 1.34 (d, J=6.7 Hz, 2H).

### Biological Assays

### Example A. Primary Assay Used to Determine Potency of MALT1 Enzymatic Activity Inhibition

Compound activity was determined using recombinant MALT1 protein (Creative Biomart, Cat #MALT1-28H) and Ac-LRSR-AMC Substrate in an in vitro enzymatic reaction. The enzymatic assay used to determine activity was a Fluorescence assay using a Microplate Reader ClarioStar Plus. The enzymatic reaction was carried out in 1× Assay Buffer (50 mM HEPES pH 7.2-7.4, 100 mM NaCl, 900 mM Sodium citrate, 10 mM DTT). The compounds were dispensed on a 384-well Diamond Well Plate (Axygen, Cat #P-384-120SQ-C—S) using the Biomek FX liquid handling system at 100× solutions of compounds in DMSO. 2.5×MALT1 mix (final concentration 1.5 ng/μl of MALT1) was prepared in 1× Assay Buffer and 8 μl of mixture per well were added into 384w white Reaction Plates with NBS (Corning, Cat #4513). 8 μl of 1× Assay Buffer were used for negative control. Plates were centrifuged for 1 min at 200 g. Next, the compounds were added to Reaction plates using Biomek station via following steps: 1 μl of 100× compounds (in DMSO) was mixed thoroughly with 19 μl of 1× Assay Buffer, then 4 μl of this mixture were added to Reaction plates with 8 μl of MALT1 mix. Plates were centrifuged for 1 min at 200 g and incubated for 10 minutes at rt. Next, 2.5× Ac-LRSR-AMC mix (final concentration 1 μM of Ac-LRSR-AMC) was prepared in 1× Assay Buffer and 8 μl of mixture per well were added to Reaction Plates using Biomek station. Plates were centrifuged for 1 min at 200 g, then the fluorescence was measured immediately using Microplate Reader. Plates were incubated for 30 minutes at 37° C., then for 30 minutes at rt. The fluorescence was measured after the whole incubation using Microplate Reader. The reaction signal was calculated as the subtraction of the first data set values from the second. The % inhibition was then used to calculate the IC50 values. The results of this assay are shown in the Table A. The values of EC₅₀ shown as a letters A-E, where: A≤0.2 μM; 0.2 μM<B≤0.5 μM; 0.5 μM<C≤1 μM; 1<D≤5 μM; E >5.

TABLE A
MALT1 enzymatic activity Inhibition

| Compound | EC₅₀, |
|---|---|
| Number | μM* |
| 1 | E |
| 2 | A |
| 3 | A |
| 4 | B |
| 5 | A |
| 6 | C |
| 8 | D |
| 9 | E |
| 10 | B |
| 11 | B |
| 12 | A |
| 13 | A |
| 14 | A |
| 16 | B |
| 17 | A |
| 18 | A |
| 19 | D |
| 20 | D |
| 21 | A |
| 22 | D |
| 23 | B |
| 24 | D |
| 25 | B |
| 26 | E |
| 27 | E |
| 28 | E |
| 29 | E |
| 31 | E |
| 32 | D |
| 33 | D |
| 34 | E |
| 35 | E |
| 36 | C |
| 37 | C |
| 38 | B |
| 39 | C |
| 40 | C |
| 41 | C |
| 42 | C |
| 43 | D |
| 44 | D |
| 45 | E |
| 46 | E |
| 47 | E |
| 48 | E |
| 49 | D |
| 50 | D |
| 51 | E |
| 52 | E |
| 54 | D |
| 55 | A |
| 56 | A |
| 57 | A |
| 58 | A |
| 59 | A |
| 60 | A |
| 61 | A |
| 62 | A |
| 64 | A |
| 65 | B |
| 66 | A |
| 67 | A |
| 68 | A |
| 70 | A |
| 71 | A |
| 73 | A |
| 74 | B |
| 75 | A |
| 76 | A |
| 77 | A |
| 78 | A |
| 79 | A |
| 80 | A |
| 81 | B |
| 82 | A |
| 83 | A |
| 84 | A |
| 85 | A |
| 86 | A |
| 87 | A |
| 88 | A |
| 89 | A |
| 91 | A |
| 92 | A |
| 93 | A |
| 94 | E |
| 95 | A |
| 96 | A |
| 97 | A |
| 98 | A |
| 99 | A |
| 100 | B |
| 101 | B |
| 102 | A |
| 103 | E |
| 104 | A |
| 105 | B |
| 106 | A |
| 107 | A |
| 108 | A |
| 109 | B |
| 110 | D |
| 111 | D |
| 112 | A |
| 114 | A |
| 115 | A |
| 116 | A |
| 118 | A |
| 119 | A |
| 120 | A |
| 122 | A |
| 123 | A |
| 124 | A |
| 125 | C |
| 126 | C |
| 127 | D |
| 128 | D |
| 129 | D |
| 130 | A |
| 131 | A |
| 132 | B |
| 133 | A |
| 134 | A |
| 135 | A |
| 136 | A |
| 137 | A |
| 138 | A |
| 139 | A |
| 140 | A |
| 141 | B |
| 142 | D |
| 143 | D |
| 144 | A |
| 145 | A |
| 146 | A |
| 147 | A |
| 148 | C |
| 149 | A |
| 150 | D |
| 151 | D |
| 152 | D |
| 153 | B |
| 154 | B |
| 155 | E |
| 156 | E |
| 157 | E |
| 158 | A |
| 159 | A |
| 160 | A |
| 161 | A |
| 162 | A |
| 164 | E |
| 165 | A |
| 166 | A |
| 169 | A |

*EC₅₀: Half maximal effective concentration: the concentration of a compound which induces a response halfway between the baseline and maximum after a specific exposure time; EC₅₀: A ≤ 0.2 μM; 0.2 μM < B ≤ 0.5 μM; 0.5 μM < C ≤ 1 μM; 1 < D ≤ 5 μM; E > 5

### Example B. IL-2 MALT1-Dependent Release Inhibition Assay in Jurkat Cells

Jurkat cells (ATCC, USA) (100,000 cells/well in a 96 well flat-bottom plate (Greiner, #655061) were cultured in RPMI 1640 supplemented with 10% FBS at 37° C., 5% CO₂ in a humidified cell culture incubator for 0.5-1 h. Then 10 μl/well 15× compound (or DMSO) was added in duplicates. Cells were incubated with compound in a humidified cell culture incubator for 0.5-1 h. After the incubation, 10 μl/well of 15× human antiCD3 HIT3a antibody (BioLegend) together with 10 μl/well of 15× human antiCD28 antibody (Invitrogen) were added to stimulate Jurkat cells for 24 h. After 24 h of stimulation, supernatants were collected and IL-2 levels in supernatants were assessed using IL-2 ELISA kit (Vector-Best, Russia).

The results of these assays are shown in the Table B.

TABLE B
IL-2 MALT1-dependent release inhibition assay in Jurkat cells

| Compound | EC₅₀,* | Compound | EC₅₀,* | Compound | EC₅₀,* | Compound | CC₅₀,* |
|---|---|---|---|---|---|---|---|
| Number | μM | Number | μM | Number | μM | Number | μM |
| 2 | B | 38 | C | 87 | A | 109 | A |
| 3 | A | 54 | D | 88 | C | 110 | D |
| 10 | B | 55 | B | 89 | A | 111 | D |
| 12 | C | 56 | A | 91 | B | 112 | D |
| 13 | D | 57 | C | 92 | A | 116 | A |
| 14 | C | 76 | B | 93 | A | 118 | A |
| 16 | C | 78 | A | 96 | A | 119 | A |
| 17 | A | 79 | A | 104 | A | 120 | A |
| 18 | A | 82 | C | 106 | A | 122 | A |
| 23 | A | 84 | C | 107 | C | 123 | A |
| 37 | C | 86 | C | 108 | A | 124 | C |

*EC₅₀: Half maximal effective concentration: the concentration of a compound which induces a response halfway between the baseline and maximum after a specific exposure time; EC₅₀: A ≤ 0.5 μM; 0.5 μM < B ≤ 1.0 μM; 1.0 μM < C ≤ 5.0 μM; 5.0 < D

### Example C. MALT1 Degradation

JeKo-1 cells (ATCC, USA) were cultured in RPMI 1640 supplemented with 10% FBS at 37° C., 5% CO₂ in a humidified cell culture incubator. 1 ml of cells (1*10⁶ cells per well) was transferred to each well of a 24-well plate (Greiner, #662160). Then 40 μl/well 25× compound (or DMSO) were added. Cells were incubated with compound in a humidified cell culture incubator for 48 h. At the end of incubation, cell suspension was transferred from wells into 2 ml tubes (Greiner, #623201), centrifuged at 300 g for 5 min and the supernatant was discarded. Then cell pellets were washed with cooled PBS. Cell lysates were prepared by adding 40 μl of RIPA lysis buffer (ProteinSimple, #040-483) to the cell pellet. After that, cell lysates were frozen and thawed twice at −80° C. and centrifuged at 20000 g for 10 minutes at +4° C. The supernatant was carefully aspirated and stored at −80° C. for further manipulations. Next, protein concentration in cell lysates was determined by Pierce™ Coomassie (Bradford) Protein Assay Kit (Thermo Scientific, #23200) according to manufacturer's instructions.

Levels of MALT1, Bcl10 and CYLD proteins in prepared cell lysates were assessed in capillary Western blotting system Jess (JS-3030, ProteinSimple) using 12-230 kDa Separation Module (ProteinSimple #SM-W004), Anti-Mouse Detection Module (ProteinSimple #DM-002) and Anti-Rabbit Detection Module (ProteinSimple #DM-001) according to manufacturer's instructions. For MALT1 detection protein concentration was adjusted to 0.1 mg/ml and anti-MALT1 primary antibodies (RnD Systems, #MAB4850) were used at 1:40 dilution. Bcl10 and CYLD were detected at 2 mg/ml protein concentration using anti-Bcl10 primary antibodies at 1:100 dilution (Abcam, #ab33905) and anti-CYLD primary antibodies at 1:40 dilution (Cell Signaling, #8462). Beta-actin was used as a loading control detected by anti-beta-actin primary antibodies (RnD Systems, #MAB8929) at 1:100 dilution.

The compound 2 showed EC₅₀ for MALT1 degradation as 0.085 μM.

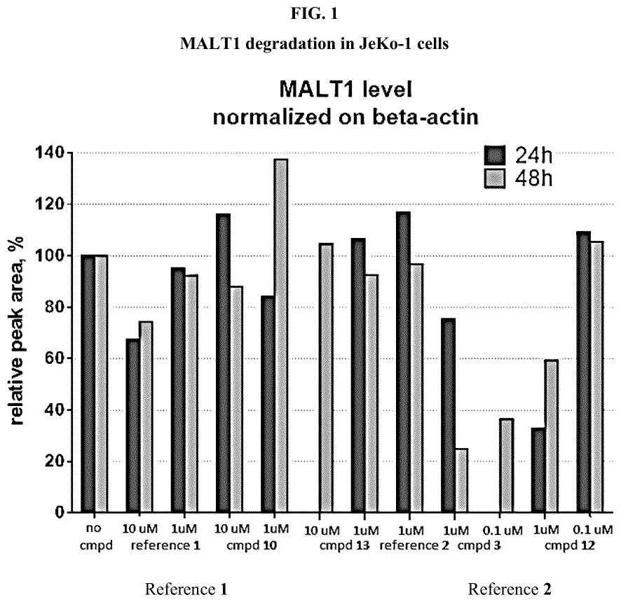

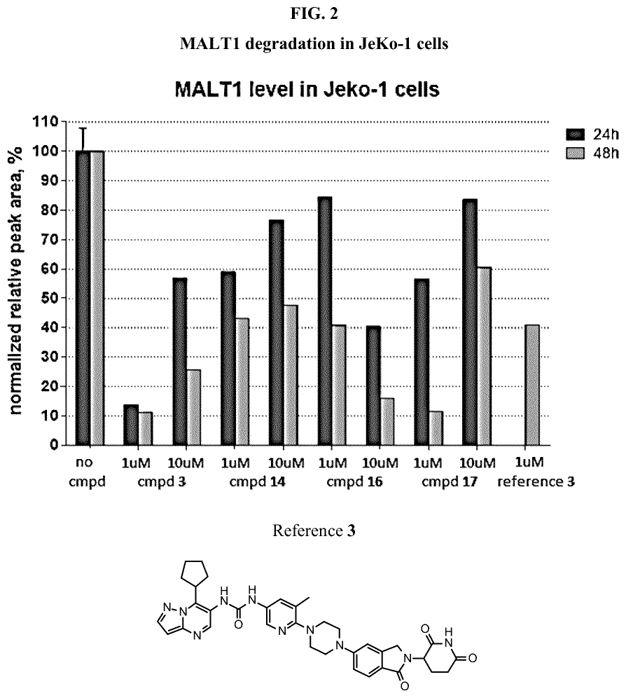

Additional experimental data for MALT1 degradation in JeKo-1 cells are shown by the bar charts of FIGS. 1 and 2.

In FIG. 1, the reference compounds were “Reference 1” and “Reference 2,” having the following structures:

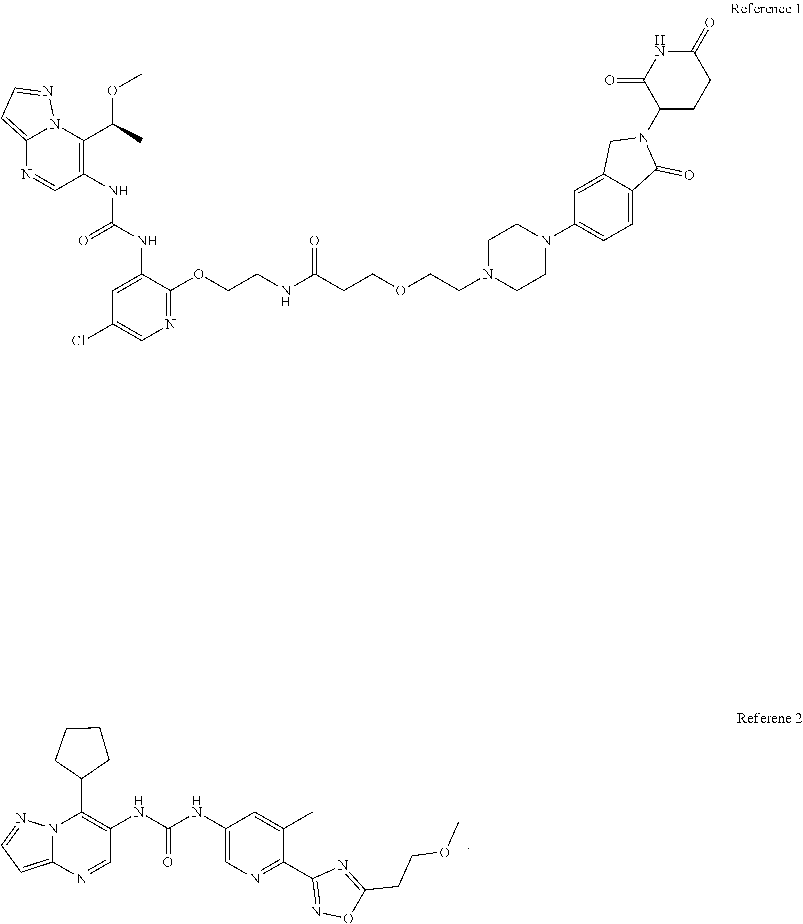 [SMILES: CO[C@@H](C)c1c(NC(=O)Nc2cc(Cl)cnc2OCCNC(=O)CCOCCN2CCN(c3ccc4c(c3)CN(C3CCC(=O)NC3=O)C4=O)CC2)cnc2ccnn12 ; COCCc1nc(-c2ncc(NC(=O)Nc3cnc4ccnn4c3C3CCCC3)cc2C)no1]

In FIG. 2, the reference compound was “Reference 3,” having the following structure:

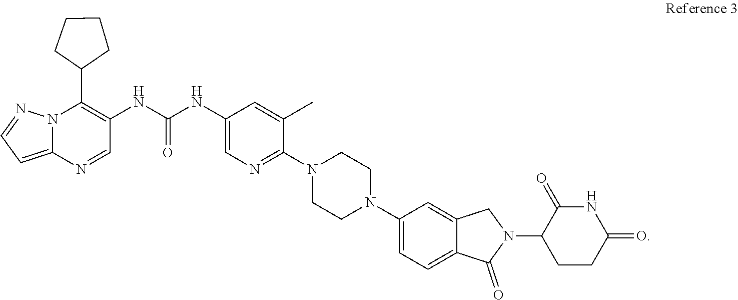 [SMILES: Cc1cc(NC(=O)Nc2cnc3ccnn3c2C2CCCC2)cnc1N1CCN(c2ccc3c(c2)CN(C2CCC(=O)NC2=O)C3=O)CC1]

FIG. 2 shows a decreasing concentration of MALT1 in Jeko-1 cells from 24 hours to 48 hours.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (A): 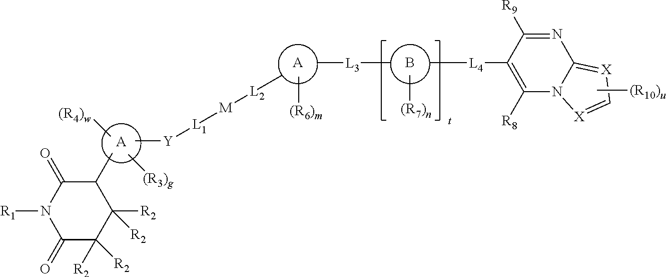
or a pharmaceutically acceptable salt, stereoisomer, solvate, or tautomer thereof,
wherein:
each X is independently selected from N and CH;
Y is selected from 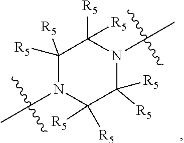
—NR₅C(O)NR₅—, and —NR₅C(O)—;
Ring A is selected from cycloalkyl, aryl, heterocyclyl, and heteroaryl;
Ring B is selected from cycloalkyl, aryl, heterocyclyl, and heteroaryl;
Ring C is selected from cycloalkyl, aryl, heterocyclyl, and heteroaryl;
M is selected from —CH₂—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
L₁ and L₂ are each independently selected from bond, C₁-C₁₂ alkanediyl, C₂-C₁₂ alkenediyl, C₂-C₁₂ alkynediyl, C₃-C₈ cycloalkanediyl, C₃-C₈ cycloalkanediyl-(CH₂)ₚ—, C₁-C₁₂ alkoxylenyl, and —((CH₂)₁₋₆O)ₒ—(CH₂)ₚ—; or
M and L₂ together is a bond; or
L₁-M-L₂ is a bond;
L₃ and L₄ are each independently selected from bond, C₁-C₁₂ alkylenyl, C₂-C₁₂ alkendiyl, C₂-C₁₂ alkyndiyl, C₁-C₁₂ alkoxylenyl, —((CH₂)₁₋₆O)ₒ—(CH₂)ₚ—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
R₁ is selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, —CH₂—OC(O)C₁-C₆ alkyl, —CH₂—O—P(O)(OC₁-C₆ alkyl)₂, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R₁₁;
each R₂, R₃, and R₉ is independently selected from hydrogen, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁; or
two R₃ are ═O;
each R₄, R₆, R₇, and R₁₀ is independently selected from deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁;
each R₅ is independently selected from hydrogen, deuterium, C₁-C₆ alkyl;
R₈ is selected from H, C₁-C₆ alkyl, cycloalkyl, aryl, heterocyclyl, and heteroaryl, wherein the alkyl, cycloalkyl, aryl, heterocycloalkyl, or heteroaryl is optionally substituted with one or more R₁₁;
each R₁₁ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl;
each R_{L} is independently selected from hydrogen, deuterium, C₁-C₆ alkyl, C₂-C₆ alkenyl, and C₂-C₆ alkynyl;
R₁₂ and R₁₃ are each independently selected from hydrogen, C₁-C₆ alkyl, C₂-C₆ alkenyl, C₂-C₆ alkynyl, C₁-C₆ haloalkyl, C₁-C₆ hydroxyalkyl, cycloalkyl, aryl, heterocyclyl, and heteroaryl; and
g is an integer selected from 0, 1, and 2;
m and n are integers each independently selected from 0, 1, 2, and 3;
o is an integer selected from 0, 1, 2, 3, 4, 5, 6, 7, 8, 9, and 10;
each p is an integer independently selected from 0, 1, 2, 3, 4, 5, 6, 7, 8, 9, and 10;
t is an integer selected from 0 and 1;
u is an integer selected from 0, 1 and 2;
w is an integer selected from 0, 1 and 2;
provided that if t is 0, then L₄ is a bond;
provided that at least one of L₃ and L₄ is not a bond,
wherein,
cycloalkyl is a saturated or partially unsaturated C₃-C₁₀ monocyclic, C₅-C₁₈ bicyclic (fused, bridged or spiro) or C₆-C₁₈ tricyclic (fused, bridged or spiro) ring system;
aryl is a cyclic, aromatic hydrocarbon ring system comprising 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) comprising 1-7 heteroatoms selected from O, N, S, P, Se, or B;
heteroaryl is a monovalent monocyclic or a polycyclic aromatic ring system comprising 5 to 24 ring atoms, wherein 1-7 ring atoms are heteroatoms selected from N, O, S, P, or B, the remaining ring atoms being C.

2. The compound of claim 1, wherein the compound is of Formula (I): 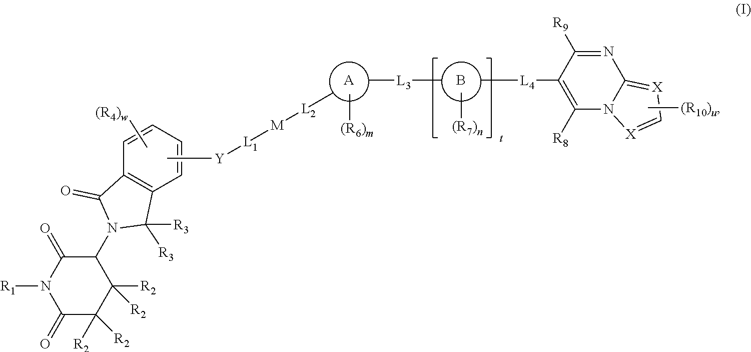
or a pharmaceutically acceptable salt, stereoisomer, solvate, or tautomer thereof,
wherein:
each X is independently selected from N and CH;
Y is selected from 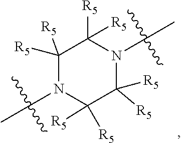
—NR₅C(O)NR₅—, and —NR₅C(O)—;
Ring A is selected from cycloalkyl, aryl, heterocyclyl, and heteroaryl;
Ring B is selected from cycloalkyl, aryl, heterocyclyl, and heteroaryl;
M is selected from —CH₂—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
L₁ and L₂ are each independently selected from bond, C₁-C₁₂ alkanediyl, C₂-C₁₂ alkenediyl, C₂-C₁₂ alkynediyl, C₃-C₈ cycloalkanediyl, C₃-C₈ cycloalkanediyl-(CH₂)ₚ—, C₁-C₁₂ alkoxylenyl, and —((CH₂)₁₋₆O)ₒ—(CH₂)ₚ—; or
M and L₂ together is a bond; or
L₁-M-L₂ is a bond;
L₃ and L₄ are each independently selected from bond, C₁-C₁₂ alkylenyl, C₂-C₁₂ alkenediyl, C₂-C₁₂ alkynediyl, C₃-C₈ cycloalkanediyl, C₁-C₁₂ alkoxylenyl, —((CH₂)₁₋₆O)ₒ—(CH₂)ₚ—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
R₁ is selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, —CH₂—OC(O)C₁-C₆ alkyl, —CH₂—O—P(O)(OC₁-C₆ alkyl)₂, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R₁₁;
each R₂, R₃, and R₉ is independently selected from hydrogen, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁; or
two R₃ are ═O;
each R₄, R₆, R₇, and R₁₀ is independently selected from deuterium, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁;
each R₅ is independently selected from hydrogen, deuterium, C₁-C₆ alkyl;
R₈ is selected from H, C₁-C₆ alkyl, cycloalkyl, aryl, heterocyclyl, and heteroaryl, wherein the alkyl, cycloalkyl, aryl, heterocycloalkyl, or heteroaryl is optionally substituted with one or more R₁₁;
each R₁₁ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl;
each R_{L} is independently selected from hydrogen, deuterium, C₁-C₆ alkyl, C₂-C₆ alkenyl, and C₂-C₆ alkynyl;
R₁₂ and R₁₃ are each independently selected from hydrogen, C₁-C₆ alkyl, C₂-C₆ alkenyl, C₂-C₆ alkynyl, C₁-C₆ haloalkyl, C₁-C₆ hydroxyalkyl, cycloalkyl, aryl, heterocyclyl, and heteroaryl; and
m and n are integers each independently selected from 0, 1, 2, and 3;
o is an integer selected from 0, 1, 2, 3, 4, 5, 6, 7, 8, 9, and 10;
each p is an integer independently selected from 0, 1, 2, 3, 4, 5, 6, 7, 8, 9, and 10;
t is an integer selected from 0 and 1;
u is an integer selected from 0, 1 and 2;
w is an integer selected from 0, 1 and 2;
provided that if t is 0, then L₄ is bond;
provided that at least one of L₃ and L₄ is not bond.

3. The compound of claim 2, wherein the compound is of Formula (I-I): 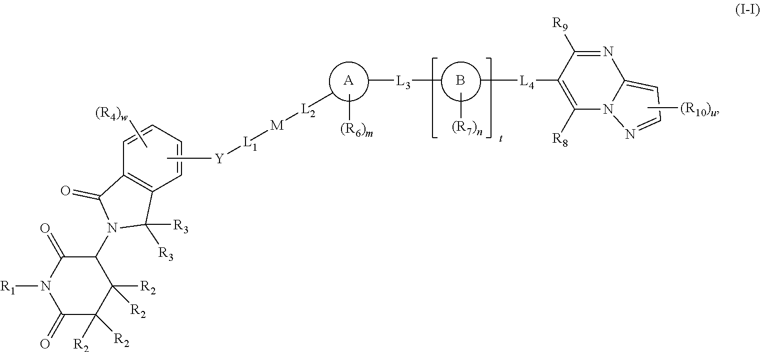
or a pharmaceutically acceptable salt, stereoisomer, solvate, or tautomer thereof.

4. The compound of claim 2, wherein the compound is of Formula (I-II): 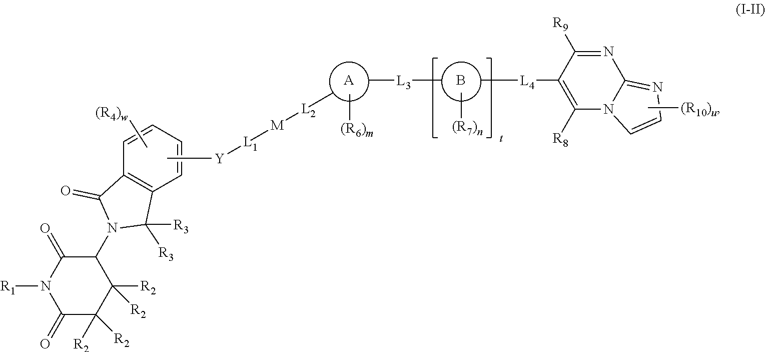
or a pharmaceutically acceptable salt, stereoisomer, solvate, or tautomer thereof.

5. The compound of claim 2, wherein the compound is of Formula (I-A) or (I-B): 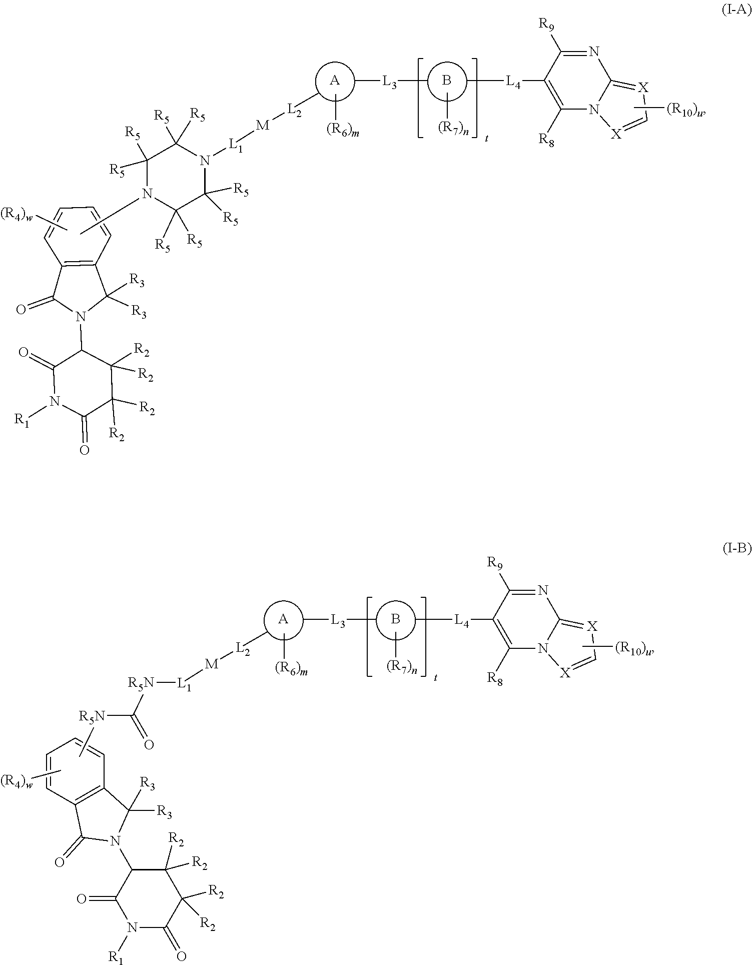
or a pharmaceutically acceptable salt, stereoisomer, solvate, or tautomer thereof.

6. The compound of claim 1,
wherein L₃ is selected from a bond and —NR_{L}C(O)NR_{L}—.

7. The compound of claim 1, wherein L₄ is selected from a bond and —NR_{L}C(O)NR_{L}—.

8. The compound of claim 1, wherein at least one from L₃ and L₄ is —NR_{L}C(O)NR_{L}— and the other one is a bond.

9. The compound of claim 1, wherein L₃ is a bond and t is 1.

10. The compound of claim 2, wherein the compound is of Formula (I-I-A-1-a-1-I-A), or (I-I-B-2-a-1-I-A): 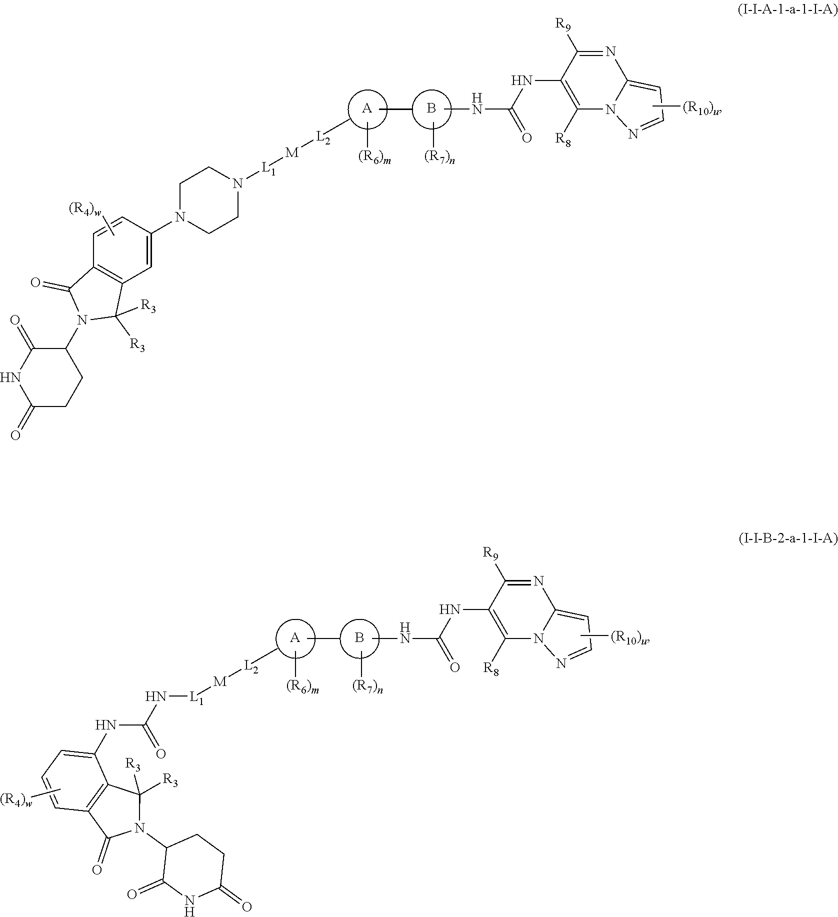
or a pharmaceutically acceptable salt, stereoisomer, solvate, or tautomer thereof.

11. The compound of claim 1, wherein Ring A is selected from 6-10 membered aryl or 5-10 membered heteroaryl comprising 1-5 heteroatoms selected from N, O, S.

12. The compound of claim 1, wherein Ring B is selected from 6-10 membered aryl or 5-10 membered heteroaryl comprising 1-5 heteroatoms selected from N, O, S.

13. The compound of claim 1, wherein R₈ is selected from C₁-C₆ alkyl, 3-10 membered cycloalkyl, 6-10 membered aryl, 3-10 membered heterocyclyl comprising 1-4 heteroatoms selected from N, O, S, and 5-10 membered heteroaryl comprising 1-5 heteroatoms selected from N, O, S, wherein the alkyl, cycloalkyl, aryl, heterocycloalkyl, or heteroaryl is optionally substituted with one or more substituents independently selected from halogen, —OH, —CN, —NO₂, —NH₂, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl.

14. The compound of claim 2, wherein the compound is of Formula (I-1), (I—2), (I-3), (I—4), (I-5), (I-6), (I-7), (I-8), (I-9), (I-10), (I-11), (I-12), (I-13), (I-14), (I-15), or (I-16): 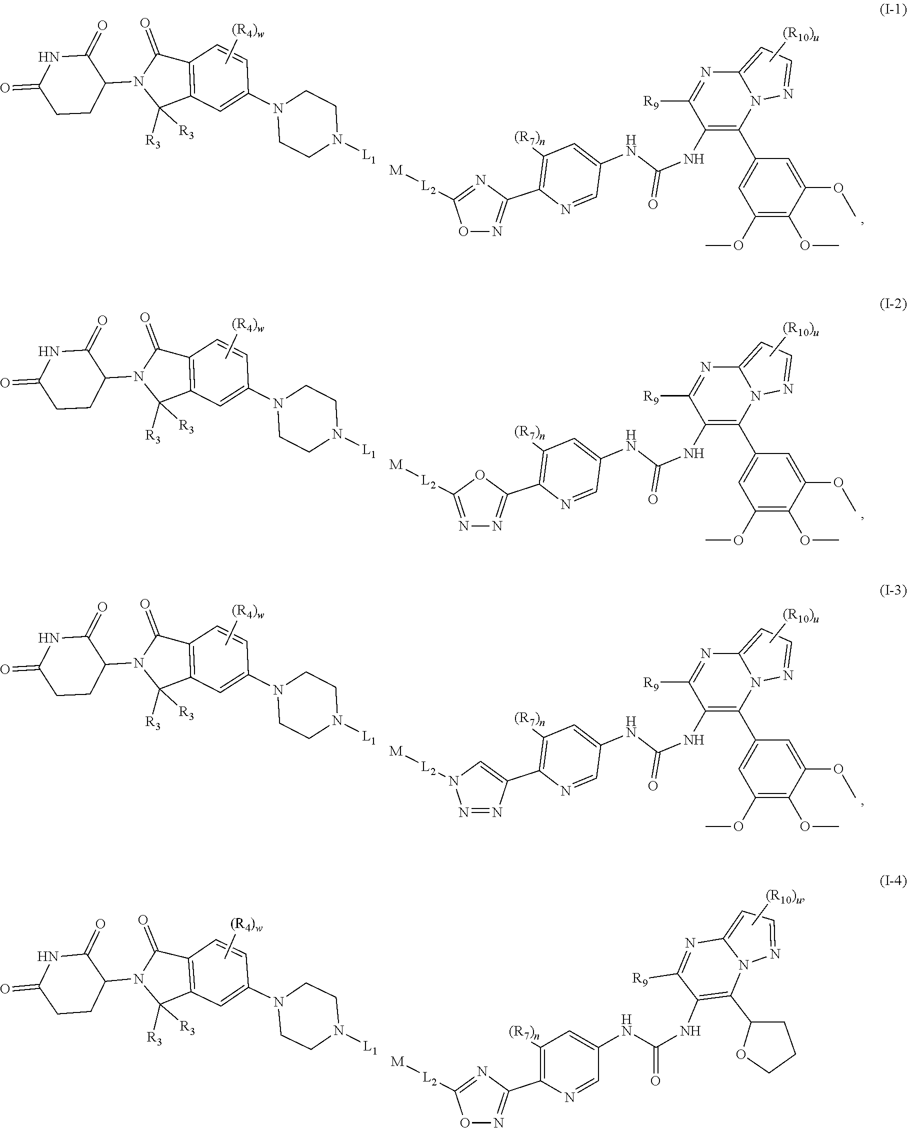 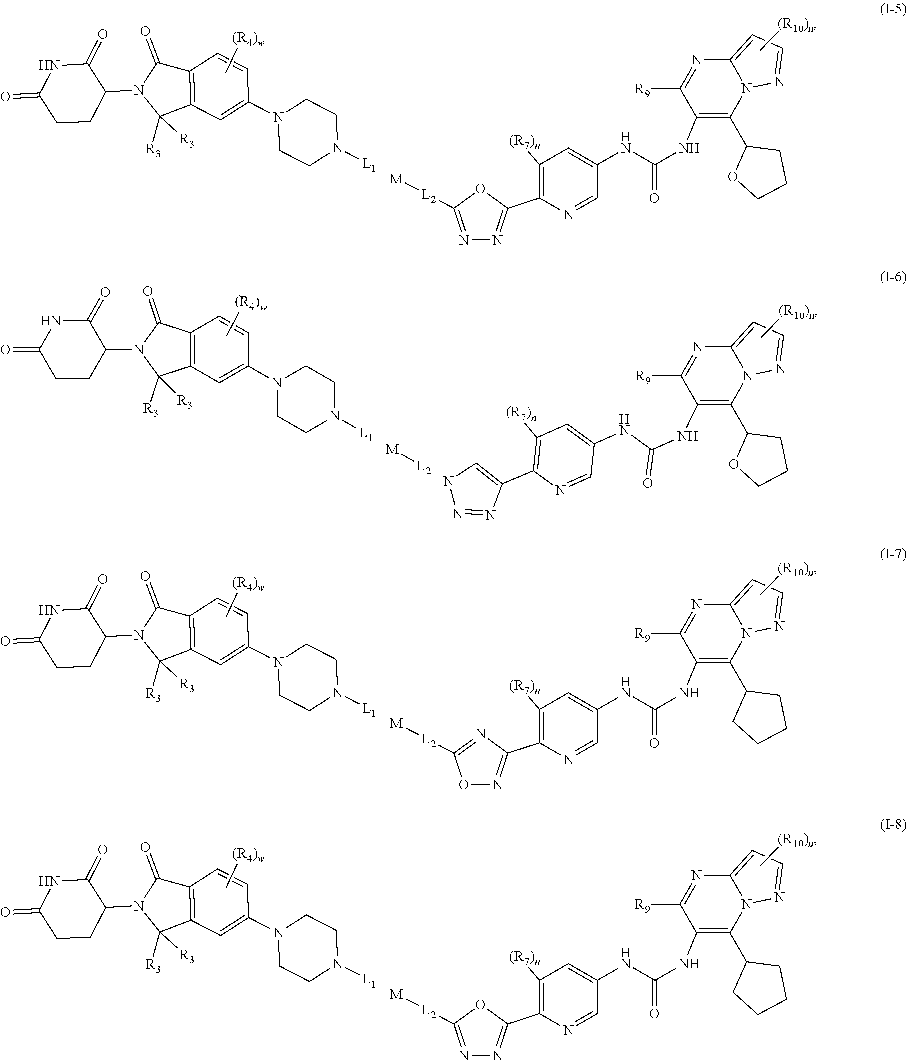 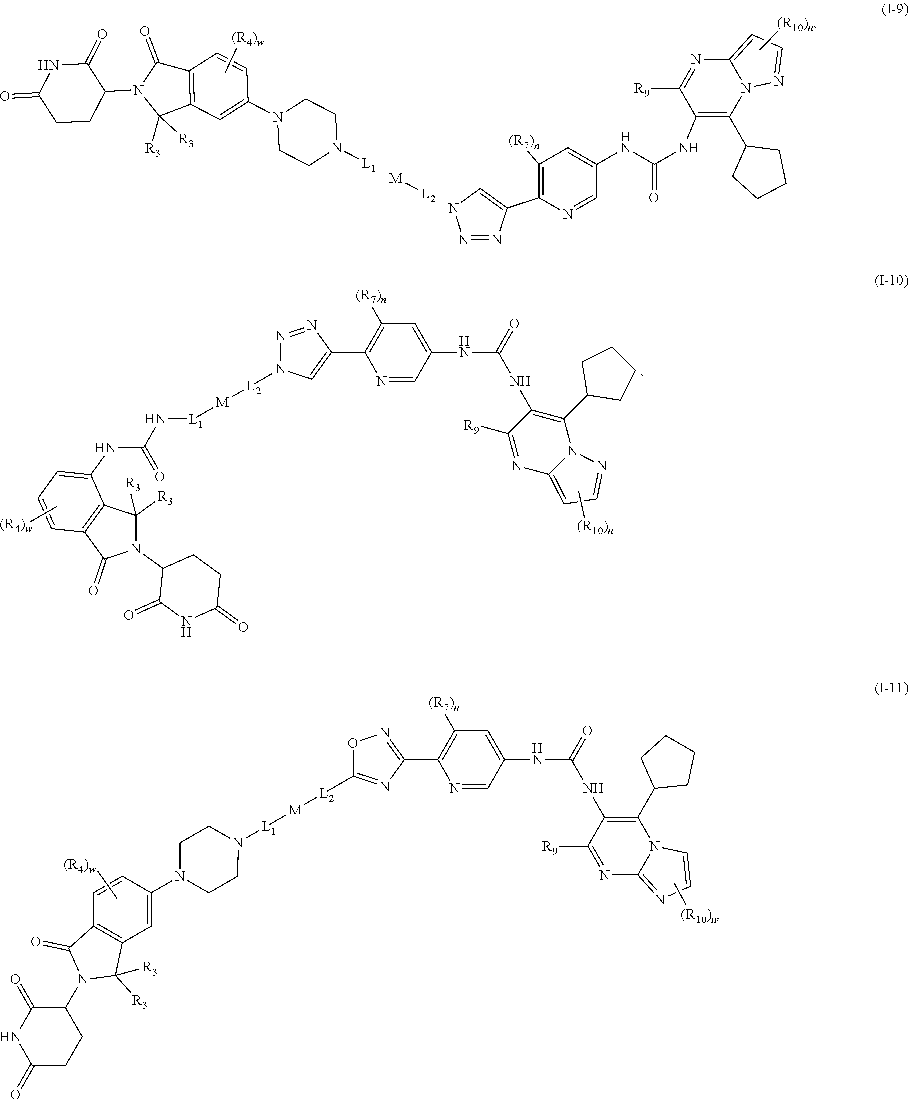 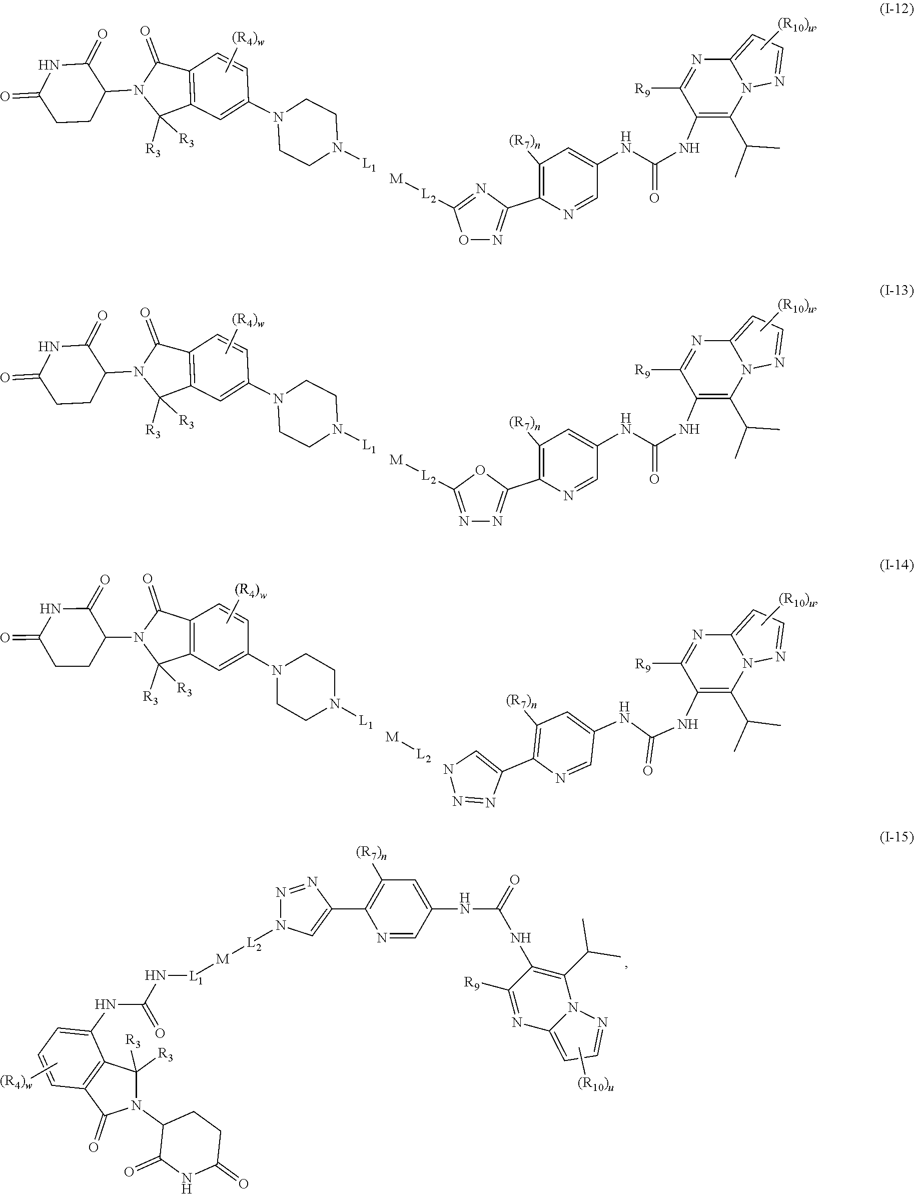 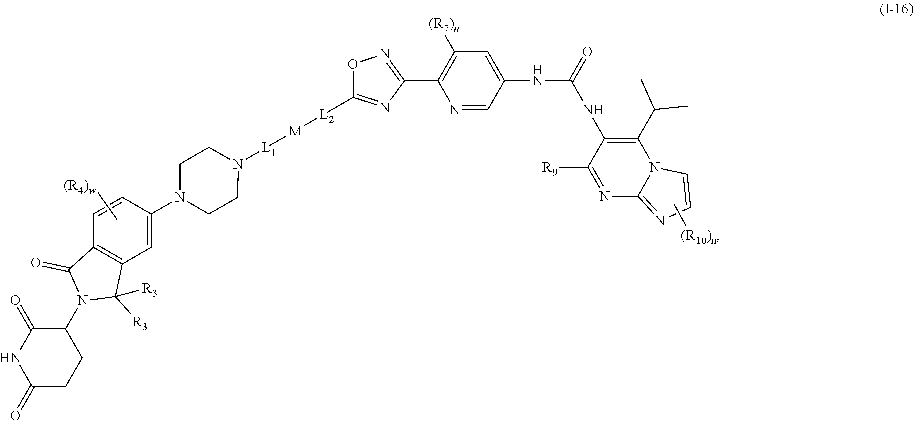
or a pharmaceutically acceptable salt, stereoisomer, solvate, or tautomer thereof, wherein n is an integer selected from 0 and 1; and wherein both R₃ are H or two R³ form ═O.

15. The compound of claim 1, wherein the compound is of Formula (II): 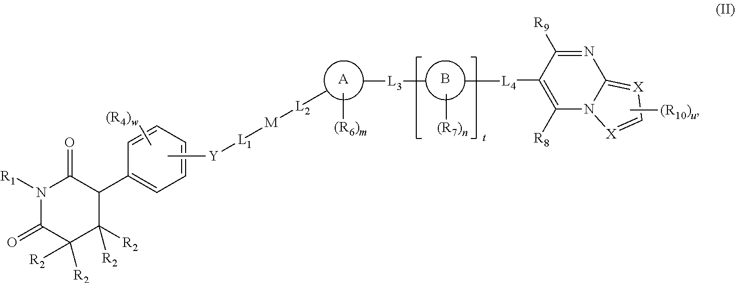
or a pharmaceutically acceptable salt, stereoisomer, solvate, or tautomer thereof,
wherein:
each X is independently selected from N and CH;
Y is selected from 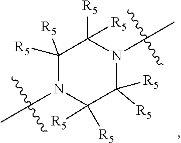
—NR₅C(O)NR₅—, and —NR₅C(O)—;
Ring A is selected from cycloalkyl, aryl, heterocyclyl, and heteroaryl;
Ring B is selected from cycloalkyl, aryl, heterocyclyl, and heteroaryl;
M is selected from —CH₂—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
L₁ and L₂ are each independently selected from bond, C₁-C₁₂ alkanediyl, C₂-C₁₂ alkendiyl, C₂-C₁₂ alkyndiyl, C₃-C₈ cycloalkanediyl, C₃-C₈ cycloalkanediyl-(CH₂)ₚ—, C₁-C₁₂ alkoxylenyl, and —((CH₂)₁₋₆O)ₒ—(CH₂)ₚ—; or
M and L₂ together is a bond; or
L₁-M-L₂ is a bond;
L₃ and L₄ are each independently selected from bond, C₁-C₁₂ alkylenyl, C₂-C₁₂ alkendiyl, C₂-C₁₂ alkyndiyl, C₁-C₁₂ alkoxylenyl, —((CH₂)₁₋₆O)ₒ—(CH₂)ₚ—, —C(O)—, —C(O)NR_{L}—, —C(O)O—, —NR_{L}—, —NR_{L}C(O)—, —NR_{L}C(O)NR_{L}—, —NR_{L}C(O)O—, —NR_{L}SO₂—, —O—, —OC(O)—, —OC(O)NR_{L}—, —OC(O)O—, —S(O)₂NR_{L}—, —S—, and —S(O)₂—;
R₁ is selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, —CH₂—OC(O)C₁-C₆ alkyl, —CH₂—O—P(O)(OC₁-C₆ alkyl)₂, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R¹¹;
each from R₂ and R₉ is independently selected from hydrogen, halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁;
each from R₄, R₆, R₇, and R₁₀ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl, wherein the alkyl, alkenyl, alkynyl, cycloalkyl, aryl, heterocyclyl, and heteroaryl are optionally substituted with one or more R₁₁;
each R₅ is independently selected from hydrogen, C₁-C₆ alkyl;
R₈ is selected from H, C₁-C₆ alkyl, cycloalkyl, aryl, heterocyclyl, and heteroaryl, wherein the alkyl, cycloalkyl, aryl, heterocycloalkyl, or heteroaryl is optionally substituted with one or more R¹¹;
each R₁₁ is independently selected from halogen, —OH, —CN, —NO₂, —NR₁₂R₁₃, C₁-C₆ alkyl, C₂-C₆ alkenyl, C₂-C₆ alkynyl, C₁-C₆ haloalkyl, C₁-C₆ alkoxy, C₁-C₆ haloalkoxy, cycloalkyl, aryl, heterocyclyl, and heteroaryl;
each R_{L} is independently selected from hydrogen, C₁-C₆ alkyl, C₂-C₆ alkenyl, and C₂-C₆ alkynyl;
R₁₂ and R₁₃ are each independently selected from hydrogen, C₁-C₆ alkyl, C₂-C₆ alkenyl, C₂-C₆ alkynyl, C₁-C₆ haloalkyl, C₁-C₆ hydroxyalkyl, cycloalkyl, aryl, heterocyclyl, and heteroaryl; and
m and n are integers each independently selected from 0, 1, 2, and 3;
o is an integer selected from 0, 1, 2, 3, 4, 5, 6, 7, 8, 9, and 10;
each p is an integer independently selected from 0, 1, 2, 3, 4, 5, 6, 7, 8, 9, and 10;
t is an integer selected from 0 and 1;
u is an integer selected from 0, 1 and 2;
w is an integer selected from 0, 1 and 2;
provided that if t is 0, then L₄ is bond;
provided that at least one of L₃ and L₄ is not a bond.

16. The compound of claim 15, wherein the compound is of Formula (II-A): 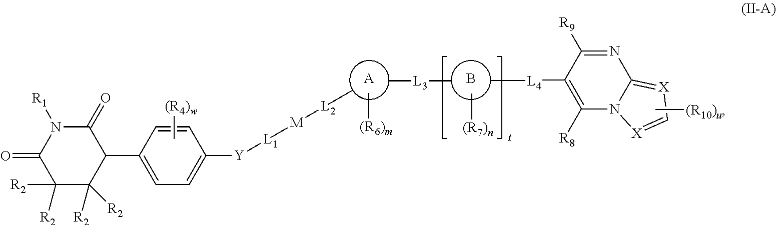
or a pharmaceutically acceptable salt, stereoisomer, solvate, or tautomer thereof.

17. The compound of claim 15, wherein the compound is of Formula (II-B): 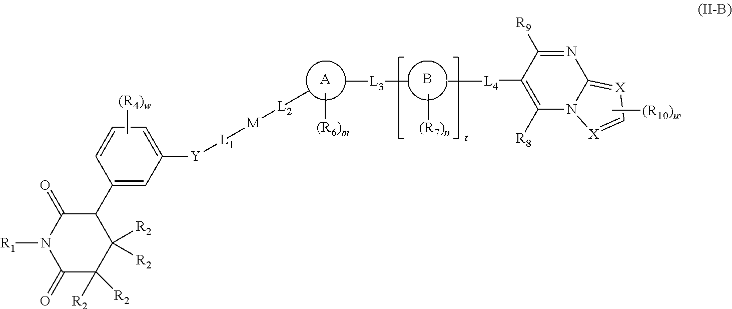
or a pharmaceutically acceptable salt, stereoisomer, solvate, or tautomer thereof.

18. A compound selected from:

| | Structure |
|---|---|
| # | IUPAC Name |
| 1 | 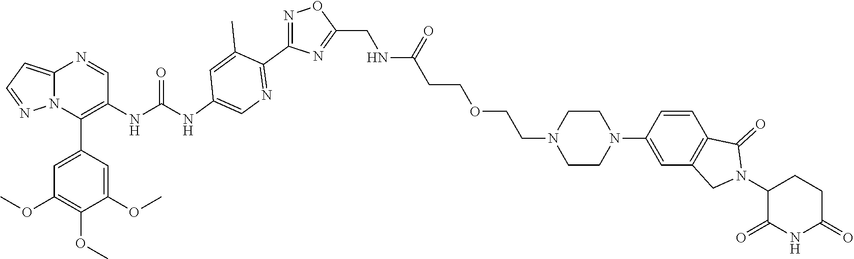 [SMILES: COc1cc(-c2c(NC(=O)Nc3cnc(-c4noc(CNC(=O)CCOCCN5CCN(c6ccc7c(c6)CN(C6CCC(=O)NC6=O)C7=O)CC5)n4)c(C)c3)cnc3ccnn23)cc(OC)c1OC] 3-[2-[4-[2-(2,6-dioxo-3-piperidyl)-1-oxo-isoindolin-5-yl]piperazin-1-yl]ethoxy]-N-[[3- [3-methyl-5-[[7-(3,4,5-trimethoxyphenyl)pyrazolo[1,5-a]pyrimidin-6- yl]carbamoylamino]-2-pyridyl]-1,2,4-oxadiazol-5-yl]methyl]propenamide |
| 2 | 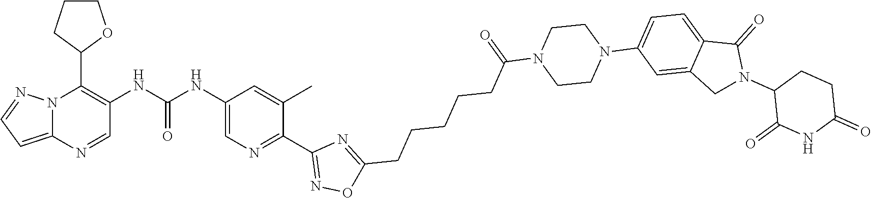 [SMILES: Cc1cc(NC(=O)Nc2cnc3ccnn3c2C2CCCO2)cnc1-c1noc(CCCCCC(=O)N2CCN(c3ccc4c(c3)CN(C3CCC(=O)NC3=O)C4=O)CC2)n1] 1-[6-[5-[6-[4-[2-(2,6-dioxo-3-piperidyl)-1-oxo-isoindolin-5-yl]piperazin-1-yl]-6-oxo- hexyl]-1,2,4-oxadiazol-3-yl]-5-methyl-3-pyridyl]-3-(7-tetrahydrofuran-2- ylpyrazolo[1,5-a]pyrimidin-6-yl)urea |
| 3 | 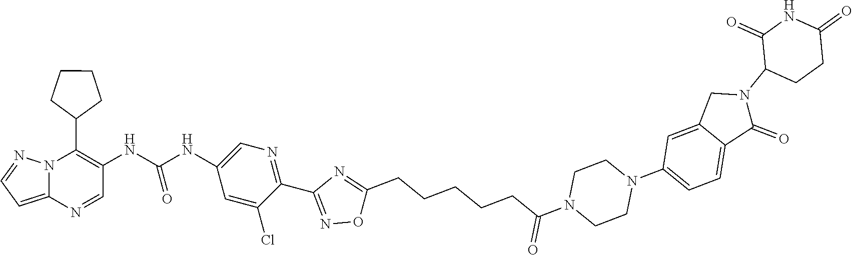 [SMILES: O=C1CCC(N2Cc3cc(N4CCN(C(=O)CCCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-cyclopentylpyrazolo[1,5- a]pyrimidin-6-yl)urea |
| 4 | 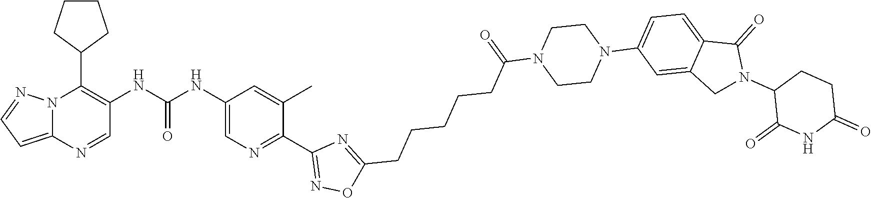 [SMILES: Cc1cc(NC(=O)Nc2cnc3ccnn3c2C2CCCC2)cnc1-c1noc(CCCCCC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 5 | 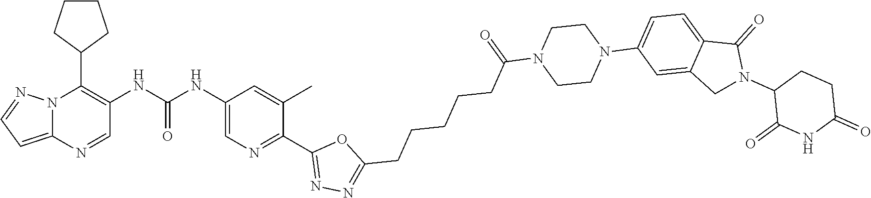 [SMILES: Cc1cc(NC(=O)Nc2cnc3ccnn3c2C2CCCC2)cnc1-c1nnc(CCCCCC(=O)N2CCN(c3ccc4c(c3)CN(C3CCC(=O)NC3=O)C4=O)CC2)o1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,3,4-oxadiazol-2-yl]-5- methyl-3-pyridyl]urea |
| 6 | 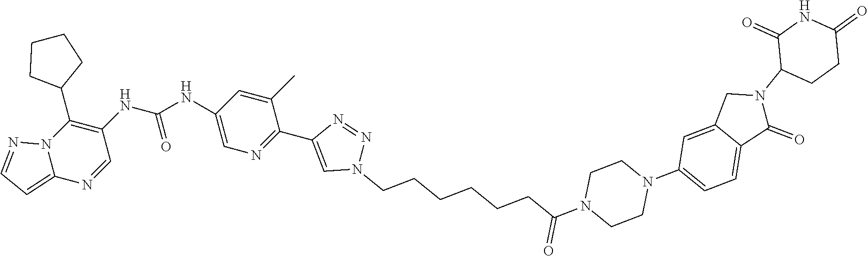 [SMILES: Cc1cc(NC(=O)Nc2cnc3ccnn3c2C2CCCC2)cnc1-c1cn(CCCCCCC(=O)N2CCN(c3ccc4c(c3)CN(C3CCC(=O)NC3=O)C4=O)CC2)nn1] 1-(7-cyclopentylpyrazolo[1,5-a]pyrimidin-6-yl)-3-[6-[1-[7-[4-[2-(2,6-dioxo-3- piperidyl)-1-oxo-isoindolin-5-yl]piperazin-1-yl]-7-oxo-heptyl]triazol-4-yl]-5-methyl-3- pyridyl]urea |
| 7 | 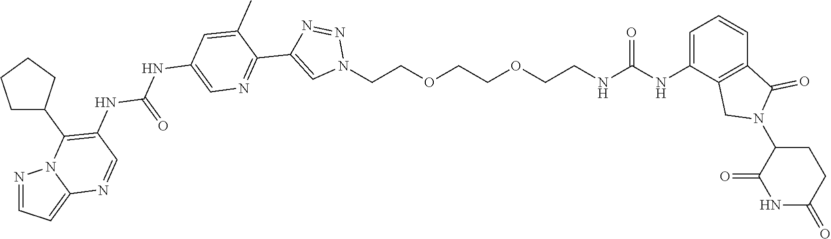 [SMILES: Cc1cc(NC(=O)Nc2cnc3ccnn3c2C2CCCC2)cnc1-c1cn(CCOCCOCCNC(=O)Nc2cccc3c2CN(C2CCC(=O)NC2=O)C3=O)nn1] 1-(7-cyclopentylpyrazolo[1,5-a]pyrimidin-6-yl)-3-[6-[1-[2-[2-[2-[[2-(2,6-dioxo-3- piperidyl)-1-oxo-isoindolin-4-yl]carbamoylamino]ethoxy]ethoxy]ethyl]triazol-4-yl]-5- methyl-3-pyridyl]urea |
| 8 | 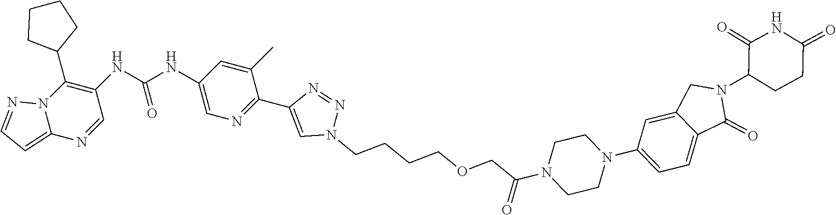 [SMILES: Cc1cc(NC(=O)Nc2cnc3ccnn3c2C2CCCC2)cnc1-c1cn(CCCCOCC(=O)N2CCN(c3ccc4c(c3)CN(C3CCC(=O)NC3=O)C4=O)CC2)nn1] 1-(7-cyclopentylpyrazolo[1,5-a]pyrimidin-6-yl)-3-[6-[1-[4-[2-[4-[2-(2,6-dioxo-3- piperidyl)-1-oxo-isoindolin-5-yl]piperazin-1-yl]-2-oxo-ethoxy]butyl]triazol-4-yl]-5- methyl-3-pyridyl]urea |
| 9 | 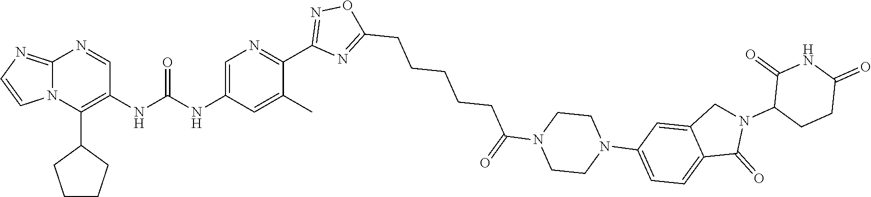 [SMILES: Cc1cc(NC(=O)Nc2cnc3nccn3c2C2CCCC2)cnc1-c1noc(CCCCCC(=O)N2CCN(c3ccc4c(c3)CN(C3CCC(=O)NC3=O)C4=O)CC2)n1] 1-(5-cyclopentylimidazo[1,2-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 10 | 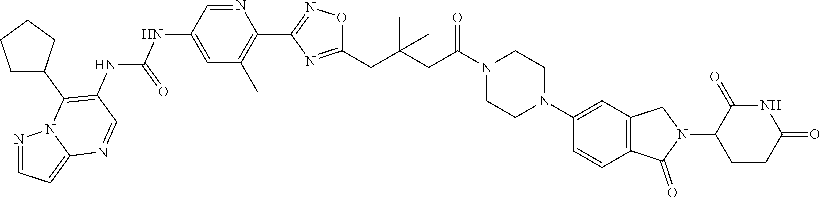 [SMILES: Cc1cc(NC(=O)Nc2cnc3ccnn3c2C2CCCC2)cnc1-c1noc(CC(C)(C)CC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-2,2-dimethyl-4-oxo-butyl]-1,2,4- oxadiazol-3-yl]-5-methyl-3-pyridyl]urea |
| 11 | 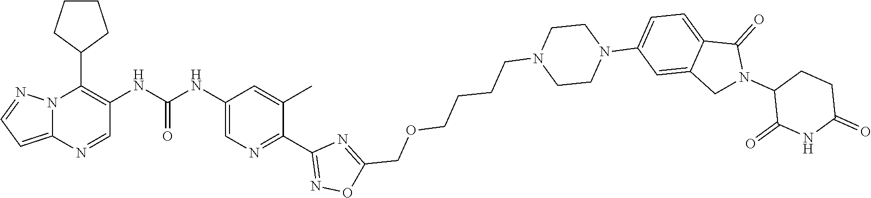 [SMILES: Cc1cc(NC(=O)Nc2cnc3ccnn3c2C2CCCC2)cnc1-c1noc(COCCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butoxymethyl]-1,2,4-oxadiazol-3-yl]- 5-methyl-3-pyridyl]urea |
| 12 | 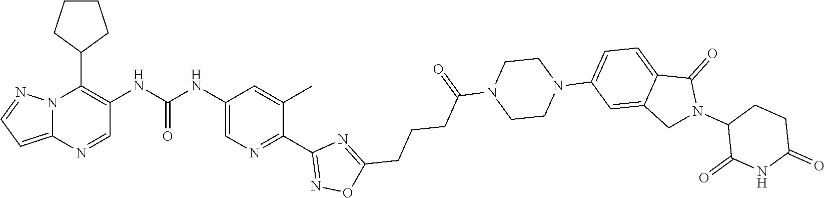 [SMILES: Cc1cc(NC(=O)Nc2cnc3ccnn3c2C2CCCC2)cnc1-c1noc(CCCC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- methyl-3-pyridyl]urea |
| 13 | 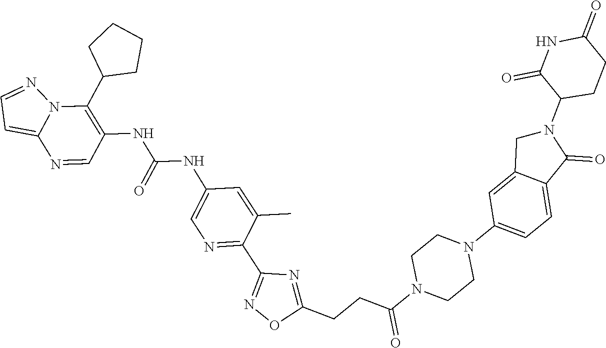 [SMILES: Cc1cc(NC(=O)Nc2cnc3ccnn3c2C2CCCC2)cnc1-c1noc(CCC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[3-[4-[2-(2,6-dioxo-3- piperidyl)-1-oxo-isoindolin-5-yl]piperazin-1-yl]-3-oxo-propyl]-1,2,4-oxadiazol-3-yl]- 5-methyl-3-pyridyl]urea |
| 14 | 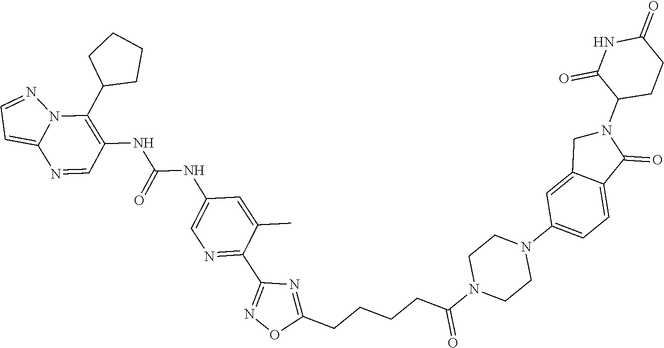 [SMILES: Cc1cc(NC(=O)Nc2cnc3ccnn3c2C2CCCC2)cnc1-c1noc(CCCCC(=O)N2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- methyl-3-pyridyl]urea |
| 15 | 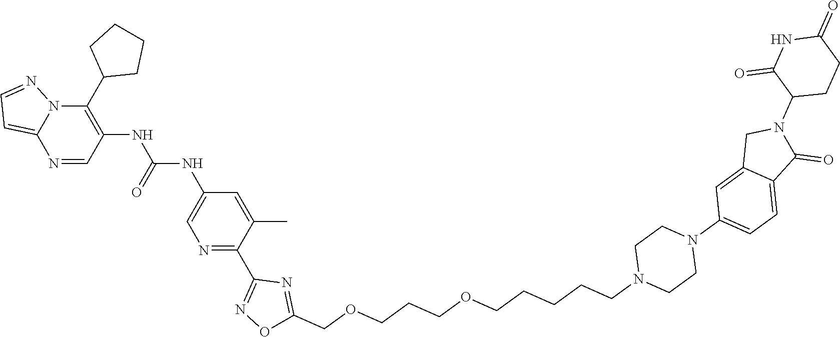 [SMILES: Cc1cc(NC(=O)Nc2cnc3ccnn3c2C2CCCC2)cnc1-c1noc(COCCCOCCCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[3-[5-[4-[2-(2,6-dioxo-3- piperidyl)-1-oxo-isoindolin-5-yl]piperazin-1-yl]pentoxy]propoxymethyl]-1,2,4- oxadiazol-3-yl]-5-methyl-3-pyridyl]urea |
| 16 | 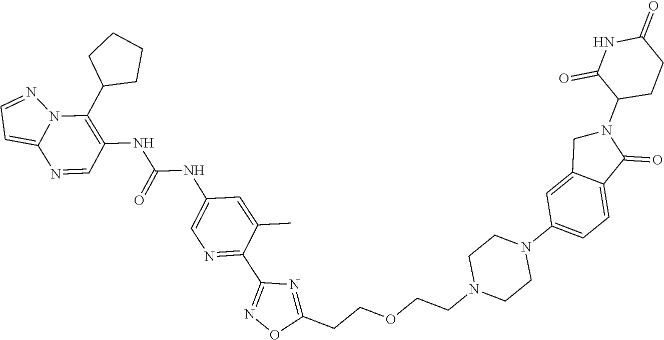 [SMILES: Cc1cc(NC(=O)Nc2cnc3ccnn3c2C2CCCC2)cnc1-c1noc(CCOCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[2-[2-[4-[2-(2,6-dioxo-3- piperidyl)-1-oxo-isoindolin-5-yl]piperazin-1-yl]ethoxy]ethyl]-1,2,4-oxadiazol-3-yl]-5- methyl-3-pyridyl]urea |
| 17 | 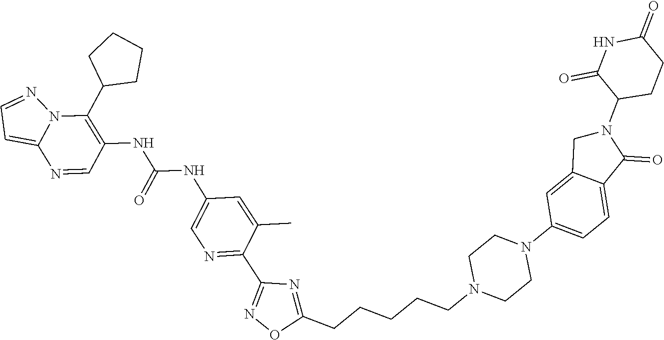 [SMILES: Cc1cc(NC(=O)Nc2cnc3ccnn3c2C2CCCC2)cnc1-c1noc(CCCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- methyl-3-pyridyl]urea |
| 18 | 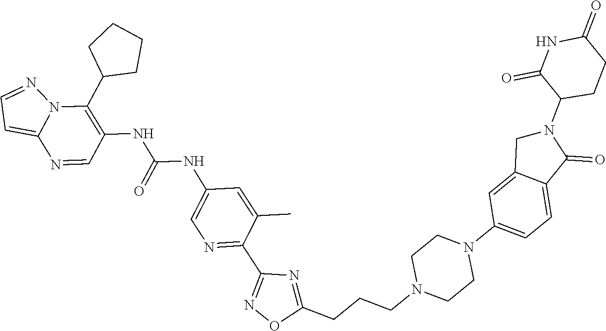 [SMILES: Cc1cc(NC(=O)Nc2cnc3ccnn3c2C2CCCC2)cnc1-c1noc(CCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[3-[4-[2-(2,6-dioxo-3- piperidyl)-1-oxo-isoindolin-5-yl]piperazin-1-yl]propyl]-1,2,4-oxadiazol-3-yl]-5- methyl-3-pyridyl]urea |
| 19 | 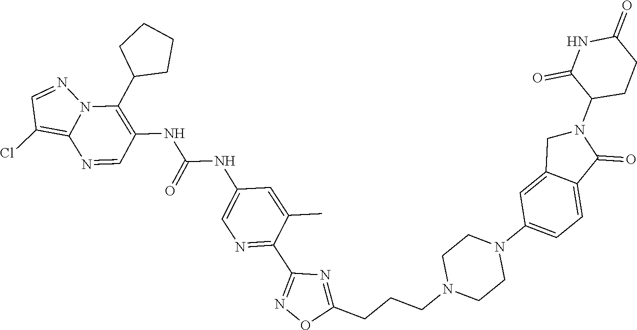 [SMILES: Cc1cc(NC(=O)Nc2cnc3c(Cl)cnn3c2C2CCCC2)cnc1-c1noc(CCCN2CCN(c3ccc4c(c3)CN(C3CCC(=O)NC3=O)C4=O)CC2)n1] 1-(3-chloro-7-cyclopentyl-pyrazolo[1,5-a]pyrimidin-6-yl)-3-[6-[5-[3-[4-[2-(2,6-dioxo- 3-piperidyl)-1-oxo-isoindolin-5-yl]piperazin-1-yl]propyl]-1,2,4-oxadiazol-3-yl]-5- methyl-3-pyridyl]urea |
| 20 | 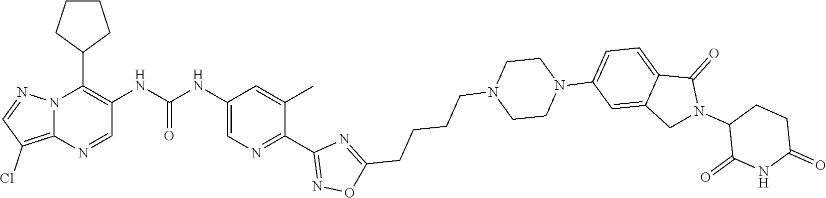 [SMILES: Cc1cc(NC(=O)Nc2cnc3c(Cl)cnn3c2C2CCCC2)cnc1-c1noc(CCCCN2CCN(c3ccc4c(c3)CN(C3CCC(=O)NC3=O)C4=O)CC2)n1] 1-(3-chloro-7-cyclopentyl-pyrazolo[1,5-a]pyrimidin-6-yl)-3-[6-[5-[4-[4-[2-(2,6-dioxo- 3-piperidyl)-1-oxo-isoindolin-5-yl]piperazin-1-yl]butyl]-1,2,4-oxadiazol-3-yl]-5- methyl-3-pyridyl]urea |
| 21 | 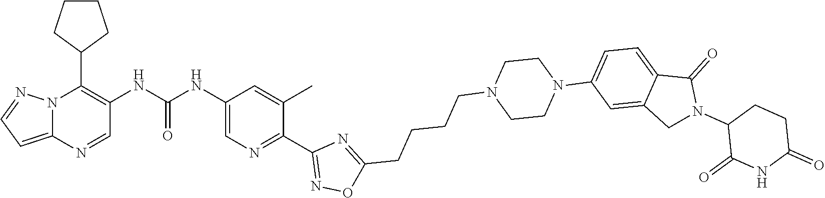 [SMILES: Cc1cc(NC(=O)Nc2cnc3ccnn3c2C2CCCC2)cnc1-c1noc(CCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butyl]-1,2,4-oxadiazol-3-yl]-5-methyl- 3-pyridyl]urea |
| 22 | 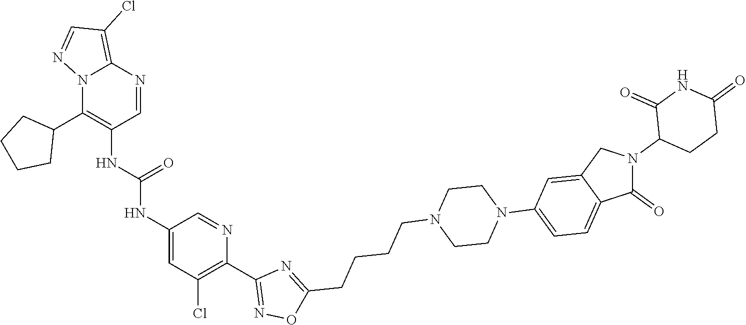 [SMILES: O=C1CCC(N2Cc3cc(N4CCN(CCCCc5nc(-c6ncc(NC(=O)Nc7cnc8c(Cl)cnn8c7C7CCCC7)cc6Cl)no5)CC4)ccc3C2=O)C(=O)N1] 1-(3-chloro-7-cyclopentyl-pyrazolo[1,5-a]pyrimidin-6-yl)-3-[5-chloro-6-[5-[4-[4-[2- (2,6-dioxo-3-piperidyl)-1-oxo-isoindolin-5-yl]piperazin-1-yl]butyl]-1,2,4-oxadiazol-3- yl]-3-pyridyl]urea |
| 23 | 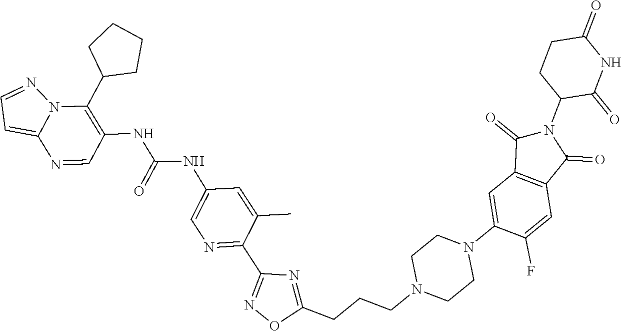 [SMILES: Cc1cc(NC(=O)Nc2cnc3ccnn3c2C2CCCC2)cnc1-c1noc(CCCN2CCN(c3cc4c(cc3F)C(=O)N(C3CCC(=O)NC3=O)C4=O)CC2)n1] 1-(7-cyclopentylpyrazolo[1,5-a]pyrimidin-6-yl)-3-[6-[5-[3-[4-[2-(2,6-dioxo-3- piperidyl)-6-fluoro-1,3-dioxo-isoindolin-5-yl]piperazin-1-yl]propyl]-1,2,4-oxadiazol-3- yl]-5-methyl-3-pyridyl]urea |
| 24 | 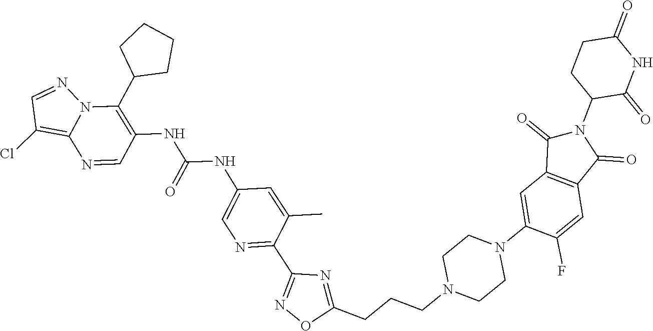 [SMILES: Cc1cc(NC(=O)Nc2cnc3c(Cl)cnn3c2C2CCCC2)cnc1-c1noc(CCCN2CCN(c3cc4c(cc3F)C(=O)N(C3CCC(=O)NC3=O)C4=O)CC2)n1] 1-(3-chloro-7-cyclopentyl-pyrazolo[1,5-a]pyrimidin-6-yl)-3-[6-[5-[3-[4-[2-(2,6-dioxo- 3-piperidyl)-6-fluoro-1,3-dioxo-isoindolin-5-yl]piperazin-1-yl]propyl]-1,2,4-oxadiazol- 3-yl]-5-methyl-3-pyridyl]urea |
| 25 | 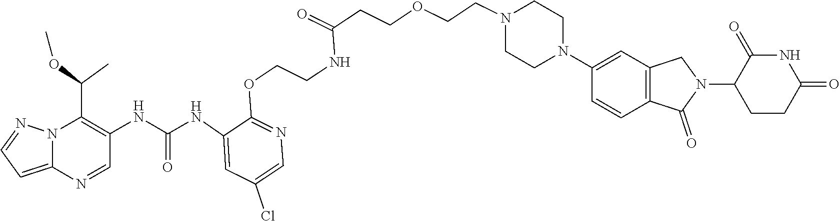 [SMILES: CO[C@@H](C)c1c(NC(=O)Nc2cc(Cl)cnc2OCCNC(=O)CCOCCN2CCN(c3ccc4c(c3)CN(C3CCC(=O)NC3=O)C4=O)CC2)cnc2ccnn12] N-[2-[[5-chloro-3-[[7-[(1S)-1-methoxyethyl]pyrazolo[1,5-a]pyrimidin-6- yl]carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 26 | 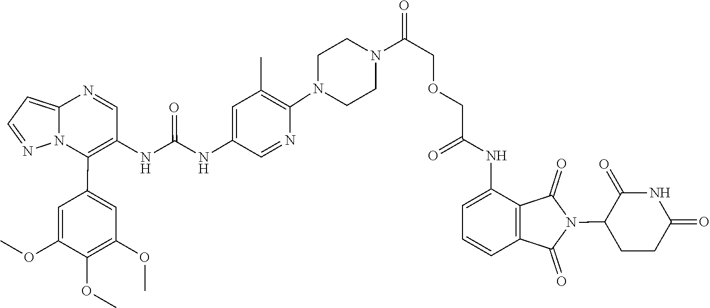 [SMILES: COc1cc(-c2c(NC(=O)Nc3cnc(N4CCN(C(=O)COCC(=O)Nc5cccc6c5C(=O)N(C5CCC(=O)NC5=O)C6=O)CC4)c(C)c3)cnc3ccnn23)cc(OC)c1OC] N-[2-(2,6-dioxo-3-piperidyl)-1,3-dioxo-isoindolin-4-yl]-2-[2-[4-[3-methyl-5-[[7- (3,4,5-trimethoxyphenyl)pyrazolo[1,5-a]pyrimidin-6-yl]carbamoylamino]-2- pyridyl]piperazin-1-yl]-2-oxo-ethoxy]acetamide |
| 27 | 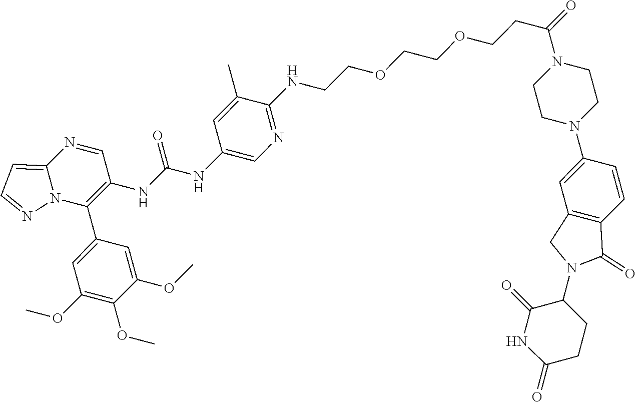 [SMILES: COc1cc(-c2c(NC(=O)Nc3cnc(NCCOCCOCCC(=O)N4CCN(c5ccc6c(c5)CN(C5CCC(=O)NC5=O)C6=O)CC4)c(C)c3)cnc3ccnn23)cc(OC)c1OC] 1-[6-[2-[2-[3-[4-[2-(2,6-dioxo-3-piperidyl)-1-oxo-isoindolin-5-yl]piperazin-1-yl]-3- oxo-propoxy]ethoxy]ethylamino]-5-methyl-3-pyridyl]-3-[7-(3,4,5- trimethoxyphenyl)pyrazolo[1,5-a]pyrimidin-6-yl]urea |
| 28 | 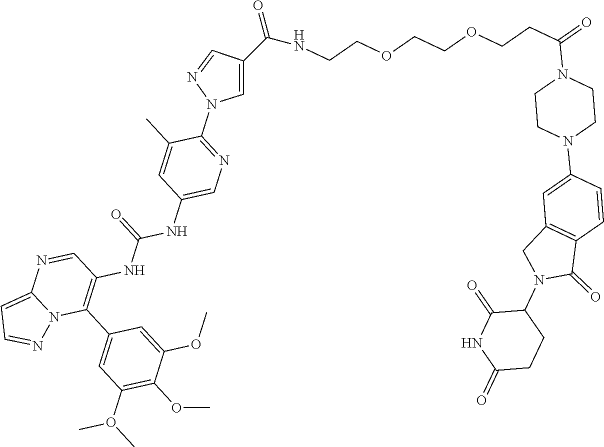 N-[2-[2-[3-[4-[2-(2,6-dioxo-3-piperidyl)-1-oxo-isoindolin-5-yl]piperazin-1-yl]-3-oxo- propoxy]ethoxy]ethyl]-1-[3-methyl-5-[[7-(3,4,5-trimethoxyphenyl)pyrazolo[1,5- a]pyrimidin-6-yl]carbamoylamino]-2-pyridyl]pyrazole-4-carboxamide |
| 29 | 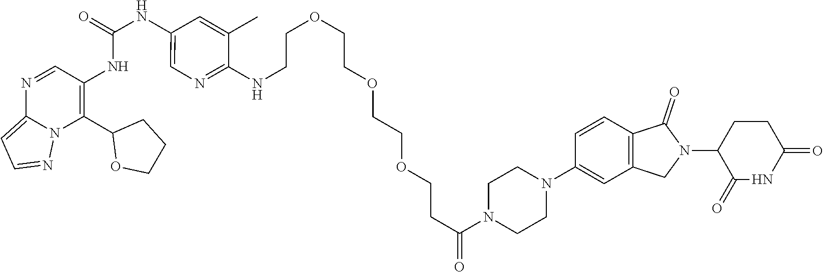 [SMILES: Cc1cc(NC(=O)Nc2cnc3ccnn3c2C2CCCO2)cnc1NCCOCCOCCOCCC(=O)N1CCN(c2ccc3c(c2)CN(C2CCC(=O)NC2=O)C3=O)CC1] 1-[6-[2-[2-[2-[3-[4-[2-(2,6-dioxo-3-piperidyl)-1-oxo-isoindolin-5-yl]piperazin-1-yl]-3- oxo-propoxy]ethoxy]ethoxy]ethylamino]-5-methyl-3-pyridyl]-3-(7-tetrahydrofuran-2- ylpyrazolo[1,5-a]pyrimidin-6-yl)urea |
| 30 | 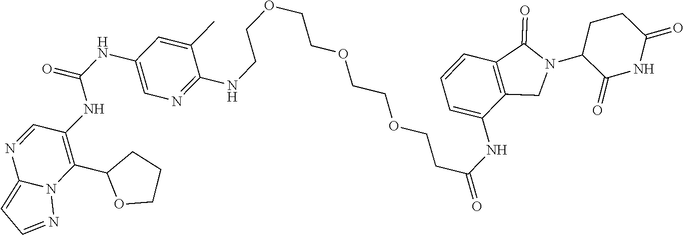 [SMILES: Cc1cc(NC(=O)Nc2cnc3ccnn3c2C2CCCO2)cnc1NCCOCCOCCOCCC(=O)Nc1cccc2c1CN(C1CCC(=O)NC1=O)C2=O] N-[2-(2,6-dioxo-3-piperidyl)-1-oxo-isoindolin-4-yl]-3-[2-[2-[2-[3-methyl-5-[(7- tetrahydrofuran-2-ylpyrazolo[1,5-a]pyrimidin-6-yl)carbamoylamino]-2- pyridyl]amino]ethoxy]ethoxy]ethoxy]propanamide |
| 31 | 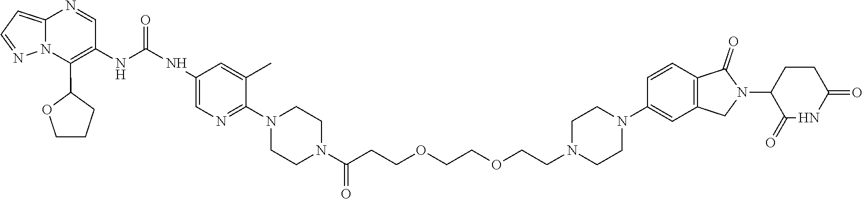 [SMILES: Cc1cc(NC(=O)Nc2cnc3ccnn3c2C2CCCO2)cnc1N1CCN(C(=O)CCOCCOCCN2CCN(c3ccc4c(c3)CN(C3CCC(=O)NC3=O)C4=O)CC2)CC1] 1-[6-[4-[3-[2-[2-[4-[2-(2,6-dioxo-3-piperidyl)-1-oxo-isoindolin-5-yl]piperazin-1- yl]ethoxy]ethoxy]propanoyl]piperazin-1-yl]-5-methyl-3-pyridyl]-3-(7-tetrahydrofuran- 2-ylpyrazolo[1,5-a]pyrimidin-6-yl)urea |
| 32 | 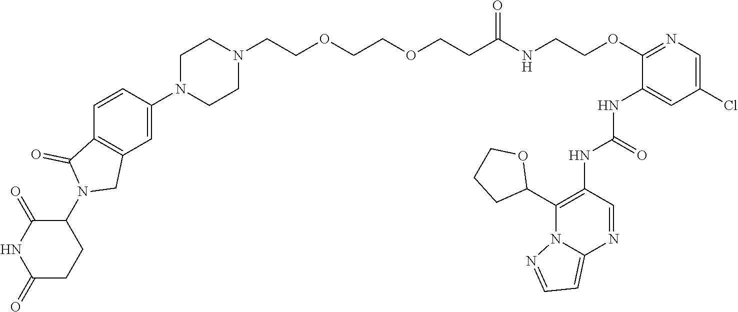 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCO1] N-[2-[[5-chloro-3-[(7-tetrahydrofuran-2-ylpyrazolo[1,5-a]pyrimidin-6- yl)carbamoylamino]-2-pyridyl]oxy]ethyl]-3-[2-[2-[4-[2-(2,6-dioxo-3-piperidyl)-1-oxo- isoindolin-5-yl]piperazin-1-yl]ethoxy]ethoxy]propanamide |
| 33 | 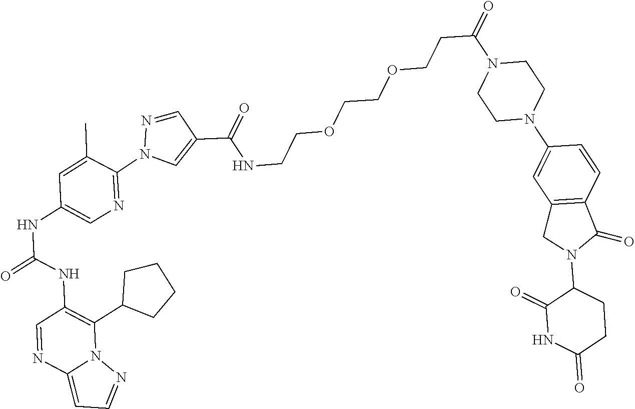 [SMILES: Cc1cc(NC(=O)Nc2cnc3ccnn3c2C2CCCC2)cnc1-n1cc(C(=O)NCCOCCOCCC(=O)N2CCN(c3ccc4c(c3)CN(C3CCC(=O)NC3=O)C4=O)CC2)cn1] N-[2-[2-[3-[4-[2-(2,6-dioxo-3-piperidyl)-1-oxo-isoindolin-5-yl]piperazin-1-yl]-3-oxo- propoxy]ethoxy]ethyl]-1-[3-methyl-5-[(7-tetrahydrofuran-2-ylpyrazolo[1,5- a]pyrimidin-6-yl)carbamoylamino]-2-pyridyl]pyrazole-4-carboxamide |
| 34 | 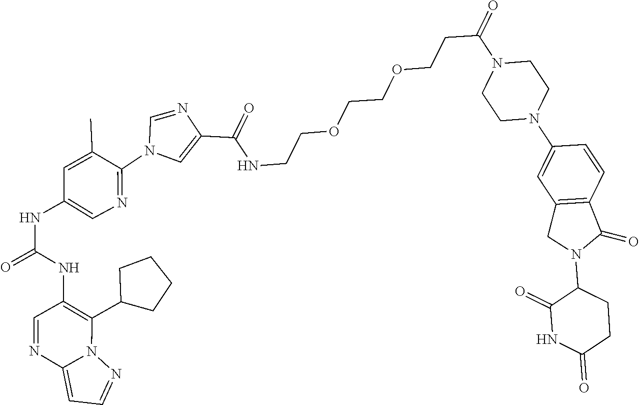 [SMILES: Cc1cc(NC(=O)Nc2cnc3ccnn3c2C2CCCC2)cnc1-n1cnc(C(=O)NCCOCCOCCC(=O)N2CCN(c3ccc4c(c3)CN(C3CCC(=O)NC3=O)C4=O)CC2)c1] N-[2-[2-[3-[4-[2-(2,6-dioxo-3-piperidyl)-1-oxo-isoindolin-5-yl]piperazin-1-yl]-3-oxo- propoxy]ethoxy]ethyl]-1-[3-methyl-5-[(7-tetrahydrofuran-2-ylpyrazolo[1,5- a]pyrimidin-6-yl)carbamoylamino]-2-pyridyl]imidazole-4-carboxamide |
| 35 | 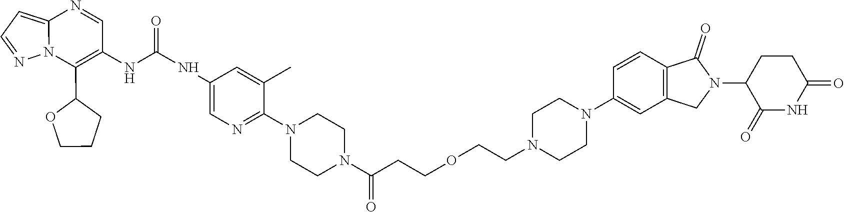 [SMILES: Cc1cc(NC(=O)Nc2cnc3ccnn3c2C2CCCO2)cnc1N1CCN(C(=O)CCOCCN2CCN(c3ccc4c(c3)CN(C3CCC(=O)NC3=O)C4=O)CC2)CC1] 1-[6-[4-[3-[2-[4-[2-(2,6-dioxo-3-piperidyl)-1-oxo-isoindolin-5-yl]piperazin-1- yl]ethoxy]propanoyl]piperazin-1-yl]-5-methyl-3-pyridyl]-3-(7-tetrahydrofuran-2- ylpyrazolo[1,5-a]pyrimidin-6-yl)urea |
| 36 | 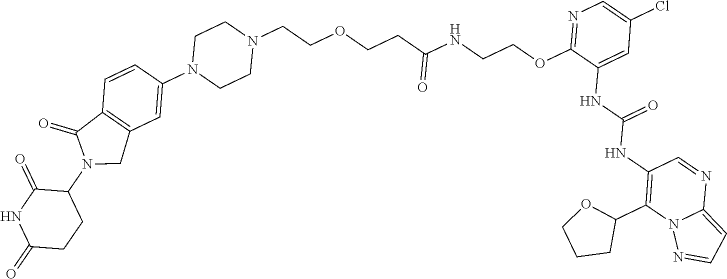 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCO1] N-[2-[[5-chloro-3-[(7-tetrahydrofuran-2-ylpyrazolo[1,5-a]pyrimidin-6- yl)carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 37 | 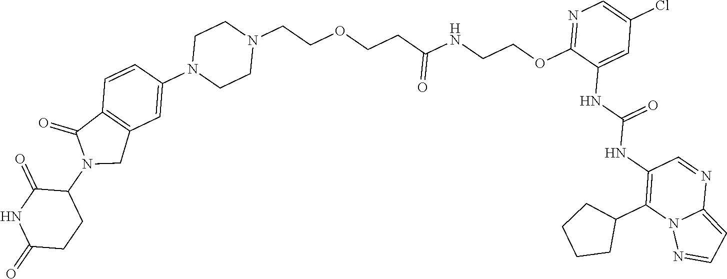 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCC1] N-[2-[[5-chloro-3-[(7-cyclopentylpyrazolo[1,5-a]pyrimidin-6-yl)carbamoylamino]-2- pyridyl]oxy]ethyl]-3-[2-[4-[2-(2,6-dioxo-3-piperidyl)-1-oxo-isoindolin-5-yl]piperazin- 1-yl]ethoxy]propanamide |
| 38 | 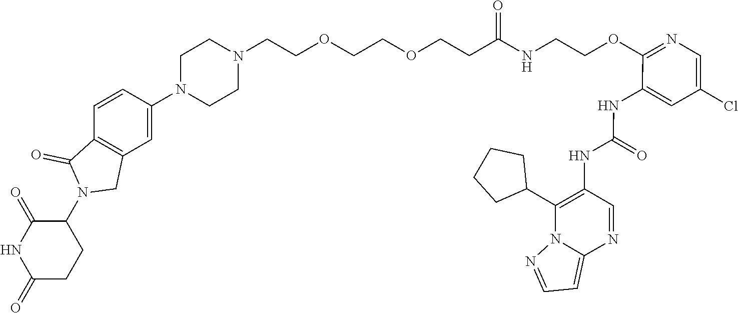 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCC1] N-[2-[[5-chloro-3-[(7-cyclopentylpyrazolo[1,5-a]pyrimidin-6-yl)carbamoylamino]-2- pyridyl]oxy]ethyl]-3-[2-[2-[4-[2-(2,6-dioxo-3-piperidyl)-1-oxo-isoindolin-5- yl]piperazin-1-yl]ethoxy]ethoxy]propanamide |
| 39 | 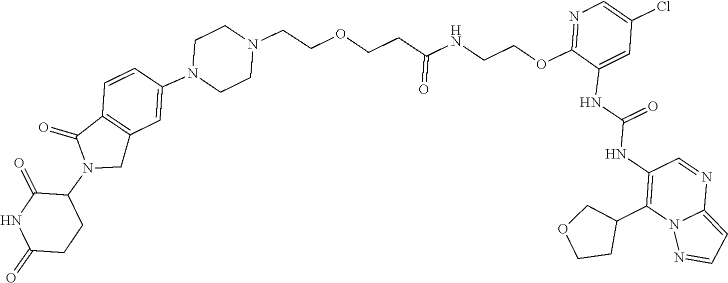 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCOC1] N-[2-[[5-chloro-3-[(7-tetrahydrofuran-3-ylpyrazolo[1,5-a]pyrimidin-6- yl)carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 40 | 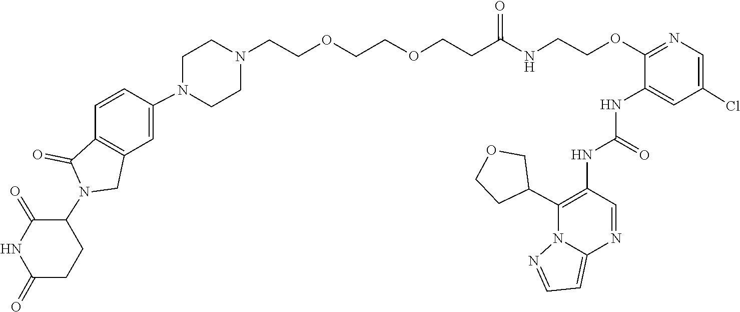 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCOC1] N-[2-[[5-chloro-3-[(7-tetrahydrofuran-3-ylpyrazolo[1,5-a]pyrimidin-6- yl)carbamoylamino]-2-pyridyl]oxy]ethyl]-3-[2-[2-[4-[2-(2,6-dioxo-3-piperidyl)-1-oxo- isoindolin-5-yl]piperazin-1-yl]ethoxy]ethoxy]propanamide |
| 41 | 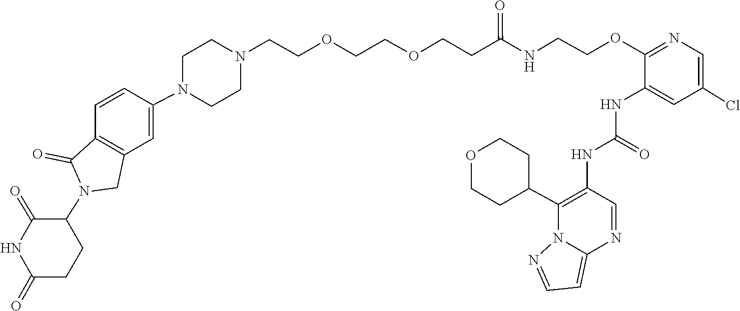 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCOCC1] N-[2-[[5-chloro-3-[(7-tetrahydropyran-4-ylpyrazolo[1,5-a]pyrimidin-6- yl)carbamoylamino]-2-pyridyl]oxy]ethyl]-3-[2-[2-[4-[2-(2,6-dioxo-3-piperidyl)-1-oxo- isoindolin-5-yl]piperazin-1-yl]ethoxy]ethoxy]propanamide |
| 42 | 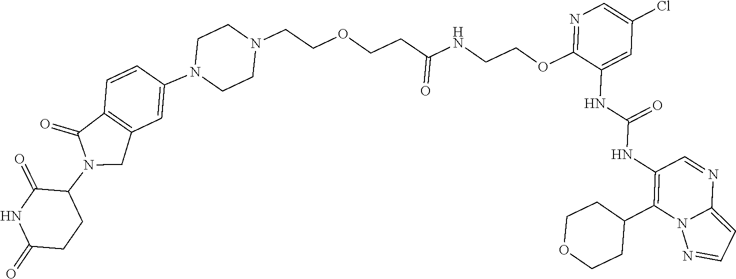 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCOCC1] N-[2-[[5-chloro-3-[(7-tetrahydropyran-4-ylpyrazolo[1,5-a]pyrimidin-6- yl)carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 43 | 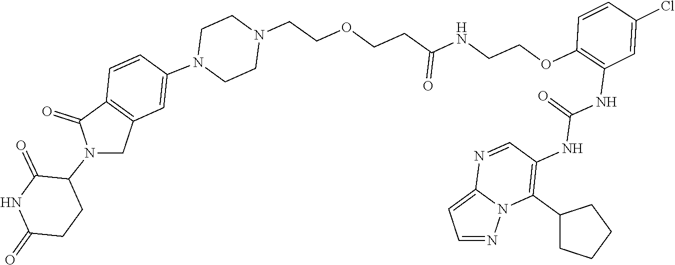 [SMILES: O=C(CCOCCN1CCN(c2ccc3c(c2)CN(C2CCC(=O)NC2=O)C3=O)CC1)NCCOc1ccc(Cl)cc1NC(=O)Nc1cnc2ccnn2c1C1CCCC1] N-[2-[4-chloro-2-[(7-cyclopentylpyrazolo[1,5-a]pyrimidin-6- yl)carbamoylamino]phenoxy]ethyl]-3-[2-[4-[2-(2,6-dioxo-3-piperidyl)-1-oxo- isoindolin-5-yl]piperazin-1-yl]ethoxy]propanamide |
| 44 | 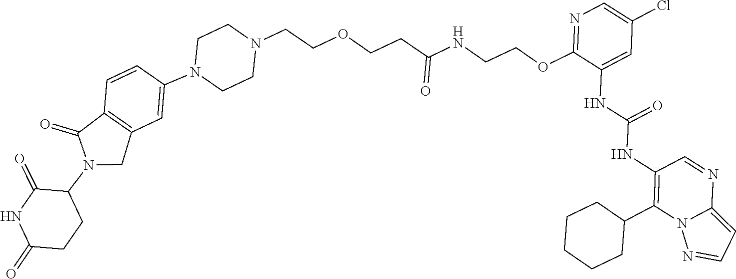 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ccnn2c1C1CCCCC1] N-[2-[[5-chloro-3-[(7-cyclohexylpyrazolo[1,5-a]pyrimidin-6-yl)carbamoylamino]-2- pyridyl]oxylethyl]-3-[2-[4-[2-(2,6-dioxo-3-piperidyl)-1-oxo-isoindolin-5-yl]piperazin- 1-yl]ethoxy]propanamide |
| 45 | 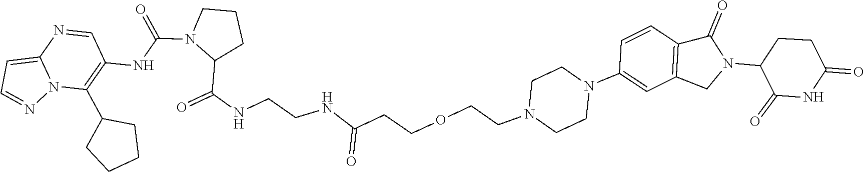 [SMILES: O=C(CCOCCN1CCN(c2ccc3c(c2)CN(C2CCC(=O)NC2=O)C3=O)CC1)NCCNC(=O)C1CCCN1C(=O)Nc1cnc2ccnn2c1C1CCCC1] N1-(7-cyclopentylpyrazolo[1,5-a]pyrimidin-6-yl)-N2-[2-[3-[2-[4-[2-(2,6-dioxo-3- piperidyl)-1-oxo-isoindolin-5-yl]piperazin-1- yl]ethoxy]propanoylamino]ethyl]pyrrolidine-1,2-dicarboxamide |
| 46 | 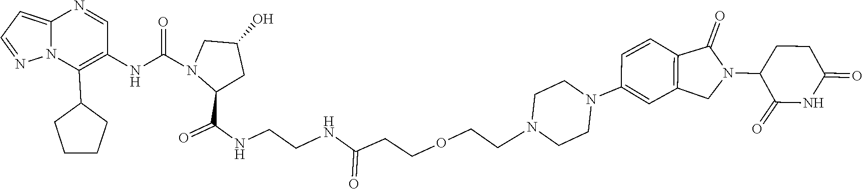 [SMILES: O=C(CCOCCN1CCN(c2ccc3c(c2)CN(C2CCC(=O)NC2=O)C3=O)CC1)NCCNC(=O)[C@@H]1C[C@@H](O)CN1C(=O)Nc1cnc2ccnn2c1C1CCCC1] (2S,4R)-N1-(7-cyclopentylpyrazolo[1,5-a]pyrimidin-6-yl)-N2-[2-[3-[2-[4-[2-(2,6- dioxo-3-piperidyl)-1-oxo-isoindolin-5-yl]piperazin-1- yl]ethoxy]propanoylamino]ethyl]-4-hydroxy-pyrrolidine-1,2-dicarboxamide |
| 47 | 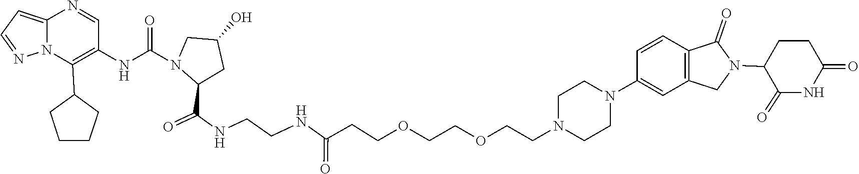 [SMILES: O=C(CCOCCOCCN1CCN(c2ccc3c(c2)CN(C2CCC(=O)NC2=O)C3=O)CC1)NCCNC(=O)[C@@H]1C[C@@H](O)CN1C(=O)Nc1cnc2ccnn2c1C1CCCC1] (2S,4R)-N1-(7-cyclopentylpyrazolo[1,5-a]pyrimidin-6-yl)-N2-[2-[3-[2-[2-[4-[2-(2,6- dioxo-3-piperidyl)-1-oxo-isoindolin-5-yl]piperazin-1- yl]ethoxy]ethoxy]propanoylamino]ethyl]-4-hydroxy-pyrrolidine-1,2-dicarboxamide |
| 48 | 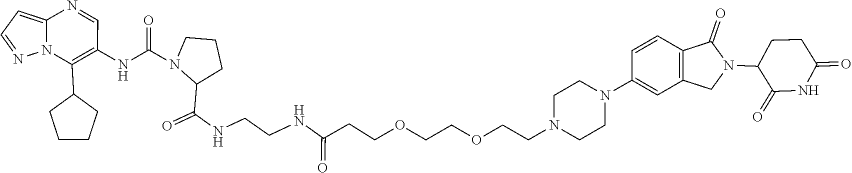 [SMILES: O=C(CCOCCOCCN1CCN(c2ccc3c(c2)CN(C2CCC(=O)NC2=O)C3=O)CC1)NCCNC(=O)C1CCCN1C(=O)Nc1cnc2ccnn2c1C1CCCC1] N1-(7-cyclopentylpyrazolo[1,5-a]pyrimidin-6-yl)-N2-[2-[3-[2-[2-[4-[2-(2,6-dioxo-3- piperidyl)-1-oxo-isoindolin-5-yl]piperazin-1- yl]ethoxy]ethoxy]propanoylamino]ethyl]pyrrolidine-1,2-dicarboxamide |
| 49 | 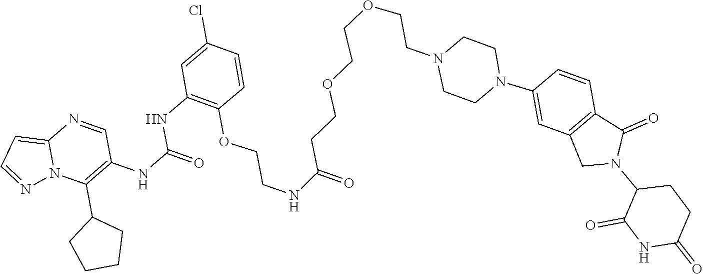 [SMILES: O=C(CCOCCOCCN1CCN(c2ccc3c(c2)CN(C2CCC(=O)NC2=O)C3=O)CC1)NCCOc1ccc(Cl)cc1NC(=O)Nc1cnc2ccnn2c1C1CCCC1] N-[2-[4-chloro-2-[(7-cyclopentylpyrazolo[1,5-a]pyrimidin-6- yl)carbamoylamino]phenoxy]ethyl]-3-[2-[2-[4-[2-(2,6-dioxo-3-piperidyl)-1-oxo- isoindolin-5-yl]piperazin-1-yl]ethoxy]ethoxy]propanamide |
| 50 | 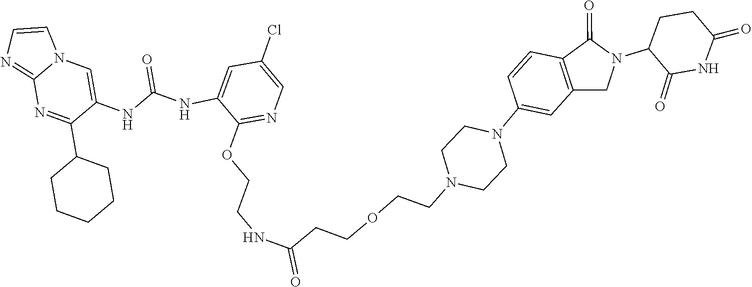 [SMILES: O=C(CCOCCN1CCN(c2ccc3c(c2)CN(C2CCC(=O)NC2=O)C3=O)CC1)NCCOc1ncc(Cl)cc1NC(=O)Nc1cn2ccnc2nc1C1CCCCC1] N-[2-[[5-chloro-3-[(7-cyclohexylimidazo[1,2-a]pyrimidin-6-yl)carbamoylamino]-2- pyridyl]oxy]ethyl]-3-[2-[4-[2-(2,6-dioxo-3-piperidyl)-1-oxo-isoindolin-5-yl]piperazin- 1-yl]ethoxy]propanamide |
| 51 | 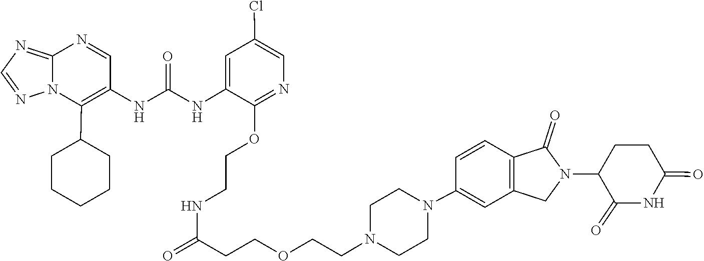 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2ncnn2c1C1CCCCC1] N-[2-[[5-chloro-3-[(7-cyclohexyl-[1,2,4]triazolo[1,5-a]pyrimidin-6- yl)carbamoylamino]-2-pyridyl]oxy]ethyl]-3-[2-[4-[2-(2,6-dioxo-3-piperidyl)-1-oxo- isoindolin-5-yl]piperazin-1-yl]ethoxy]propanamide |
| 52 | 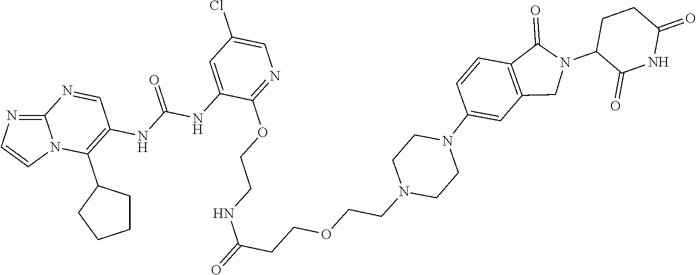 [SMILES: O=C(CCOCCN1CCN(c2ccc3c(c2)CN(C2CCC(=O)NC2=O)C3=O)CC1)NCCOc1ncc(Cl)cc1NC(=O)Nc1cnc2nccn2c1C1CCCC1] N-[2-[[5-chloro-3-[(5-cyclopentylimidazo[1,2-a]pyrimidin-6-yl)carbamoylamino]-2- pyridyl]oxy]ethyl]-3-[2-[4-[2-(2,6-dioxo-3-piperidyl)-1-oxo-isoindolin-5-yl]piperazin- 1-yl]ethoxy]propanamide |
| 53 | 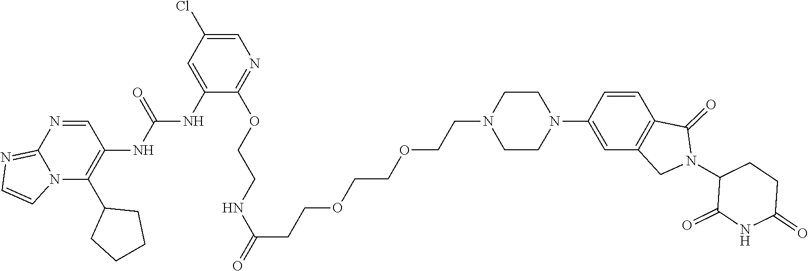 [SMILES: O=C(CCOCCOCCN1CCN(c2ccc3c(c2)CN(C2CCC(=O)NC2=O)C3=O)CC1)NCCOc1ncc(Cl)cc1NC(=O)Nc1cnc2nccn2c1C1CCCC1] N-[2-[[5-chloro-3-[(5-cyclopentylimidazo[1,2-a]pyrimidin-6-yl)carbamoylamino]-2- pyridyl]oxy]ethyl]-3-[2-[2-[4-[2-(2,6-dioxo-3-piperidyl)-1-oxo-isoindolin-5- yl]piperazin-1-yl]ethoxy]ethoxy]propanamide |
| 54 | 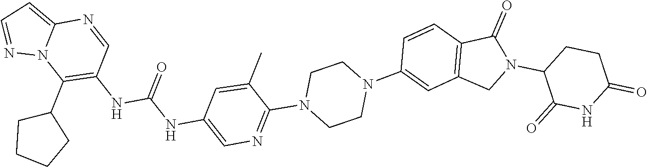 [SMILES: Cc1cc(NC(=O)Nc2cnc3ccnn3c2C2CCCC2)cnc1N1CCN(c2ccc3c(c2)CN(C2CCC(=O)NC2=O)C3=O)CC1] 1-(7-cyclopentylpyrazolo[1,5-a]pyrimidin-6-yl)-3-[6-[4-[2-(2,6-dioxo-3-piperidyl)-1- oxo-isoindolin-5-yl]piperazin-1-yl]-5-methyl-3-pyridyl]urea |
| 55 | 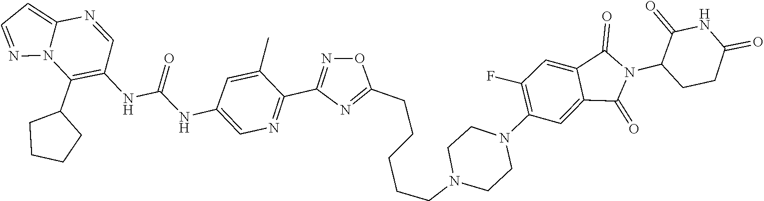 [SMILES: Cc1cc(NC(=O)Nc2cnc3ccnn3c2C2CCCC2)cnc1-c1noc(CCCCCN2CCN(c3cc4c(cc3F)C(=O)N(C3CCC(=O)NC3=O)C4=O)CC2)n1] 1-(7-cyclopentylpyrazolo[1,5-a]pyrimidin-6-yl)-3-[6-[5-[5-[4-[2-(2,6-dioxo-3- piperidyl)-6-fluoro-1,3-dioxo-isoindolin-5-yl]piperazin-1-yl]pentyl]-1,2,4-oxadiazol-3- yl]-5-methyl-3-pyridyl]urea |
| 56 | 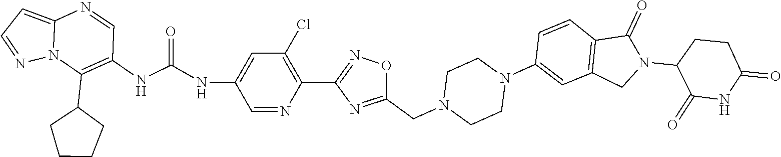 [SMILES: O=C1CCC(N2Cc3cc(N4CCN(Cc5nc(-c6ncc(NC(=O)Nc7cnc8ccnn8c7C7CCCC7)cc6Cl)no5)CC4)ccc3C2=O)C(=O)N1] 1-[5-chloro-6-[5-[[4-[2-(2,6-dioxo-3-piperidyl)-1-oxo-isoindolin-5-yl]piperazin-1- yl]methyl]-1,2,4-oxadiazol-3-yl]-3-pyridyl]-3-(7-cyclopentylpyrazolo[1,5-a]pyrimidin- 6-yl)urea |
| 57 | 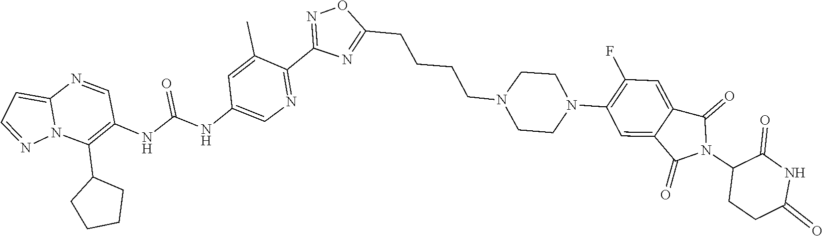 [SMILES: Cc1cc(NC(=O)Nc2cnc3ccnn3c2C2CCCC2)cnc1-c1noc(CCCCN2CCN(c3cc4c(cc3F)C(=O)N(C3CCC(=O)NC3=O)C4=O)CC2)n1] 1-(7-cyclopentylpyrazolo[1,5-a]pyrimidin-6-yl)-3-[6-[5-[4-[4-[2-(2,6-dioxo-3- piperidyl)-6-fluoro-1,3-dioxo-isoindolin-5-yl]piperazin-1-yl]butyl]-1,2,4-oxadiazol-3- yl]-5-methyl-3-pyridyl]urea |
| 58 | 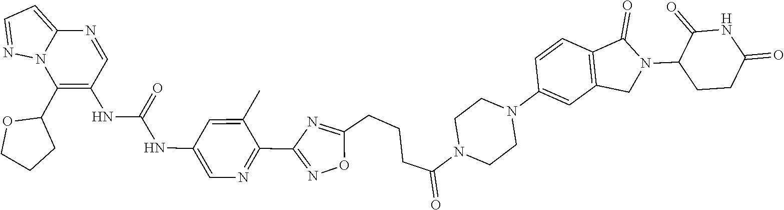 [SMILES: Cc1cc(NC(=O)Nc2cnc3ccnn3c2C2CCCO2)cnc1-c1noc(CCCC(=O)N2CCN(c3ccc4c(c3)CN(C3CCC(=O)NC3=O)C4=O)CC2)n1] 1-[6-[5-[4-[4-[2-(2,6-dioxo-3-piperidyl)-1-oxo-isoindolin-5-yl]piperazin-1-yl]-4-oxo- butyl]-1,2,4-oxadiazol-3-yl]-5-methyl-3-pyridyl]-3-(7-tetrahydrofuran-2- ylpyrazolo[1,5-a]pyrimidin-6-yl)urea |
| 59 | 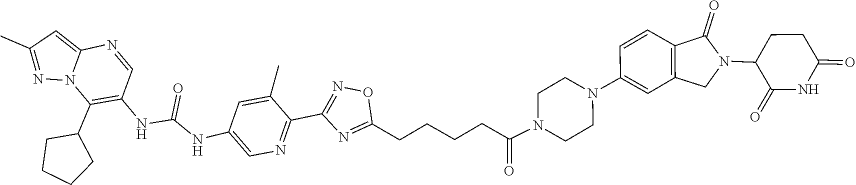 [SMILES: Cc1cc2ncc(NC(=O)Nc3cnc(-c4noc(CCCCC(=O)N5CCN(c6ccc7c(c6)CN(C6CCC(=O)NC6=O)C7=O)CC5)n4)c(C)c3)c(C3CCCC3)n2n1] 1-(7-cyclopentyl-2-methyl-pyrazolo[1,5-a]pyrimidin-6-yl)-3-[6-[5-[5-[4-[2-(2,6-dioxo- 3-piperidyl)-1-oxo-isoindolin-5-yl]piperazin-1-yl]-5-oxo-pentyl]-1,2,4-oxadiazol-3-yl]- 5-methyl-3-pyridyl]urea |
| 60 | 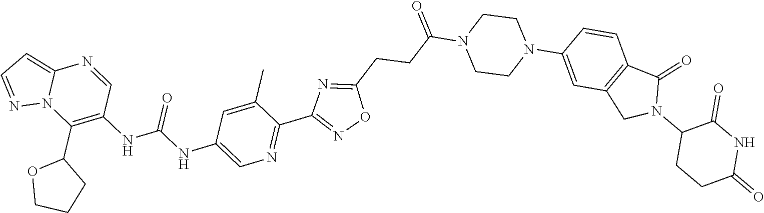 [SMILES: Cc1cc(NC(=O)Nc2cnc3ccnn3c2C2CCCO2)cnc1-c1noc(CCC(=O)N2CCN(c3ccc4c(c3)CN(C3CCC(=O)NC3=O)C4=O)CC2)n1] 1-[6-[5-[3-[4-[2-(2,6-dioxo-3-piperidyl)-1-oxo-isoindolin-5-yl]piperazin-1-yl]-3-oxo- propyl]-1,2,4-oxadiazol-3-yl]-5-methyl-3-pyridyl]-3-(7-tetrahydrofuran-2- ylpyrazolo[1,5-a]pyrimidin-6-yl)urea |
| 61 | 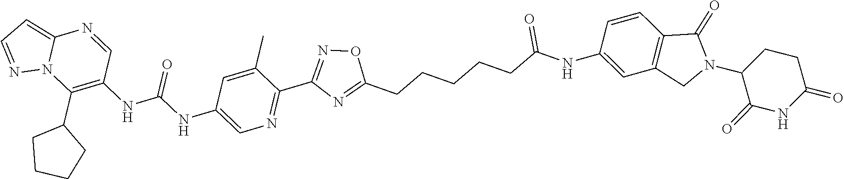 [SMILES: Cc1cc(NC(=O)Nc2cnc3ccnn3c2C2CCCC2)cnc1-c1noc(CCCCCC(=O)Nc2ccc3c(c2)CN(C2CCC(=O)NC2=O)C3=O)n1] 6-[3-[5-[(7-cyclopentylpyrazolo[1,5-a]pyrimidin-6-yl)carbamoylamino]-3-methyl-2- pyridyl]-1,2,4-oxadiazol-5-yl]-N-[2-(2,6-dioxo-3-piperidyl)-1-oxo-isoindolin-5- yl]hexanamide |
| 62 | 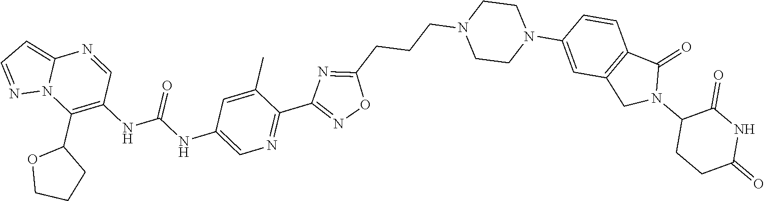 [SMILES: Cc1cc(NC(=O)Nc2cnc3ccnn3c2C2CCCO2)cnc1-c1noc(CCCN2CCN(c3ccc4c(c3)CN(C3CCC(=O)NC3=O)C4=O)CC2)n1] 1-[6-[5-[3-[4-[2-(2,6-dioxo-3-piperidyl)-1-oxo-isoindolin-5-yl]piperazin-1-yl]propyl]- 1,2,4-oxadiazol-3-yl]-5-methyl-3-pyridyl]-3-(7-tetrahydrofuran-2-ylpyrazolo[1,5- a]pyrimidin-6-yl)urea |
| 63 | 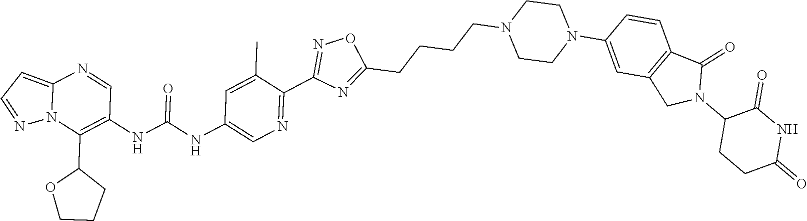 [SMILES: Cc1cc(NC(=O)Nc2cnc3ccnn3c2C2CCCO2)cnc1-c1noc(CCCCN2CCN(c3ccc4c(c3)CN(C3CCC(=O)NC3=O)C4=O)CC2)n1] 1-[6-[5-[4-[4-[2-(2,6-dioxo-3-piperidyl)-1-oxo-isoindolin-5-yl]piperazin-1-yl]butyl]- 1,2,4-oxadiazol-3-yl]-5-methyl-3-pyridyl]-3-(7-tetrahydrofuran-2-ylpyrazolo[1,5- a]pyrimidin-6-yl)urea |
| 64 | 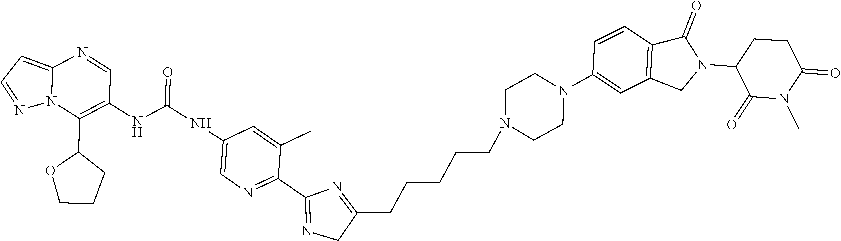 [SMILES: Cc1cc(NC(=O)Nc2cnc3ccnn3c2C2CCCO2)cnc1C1=NCC(CCCCCN2CCN(c3ccc4c(c3)CN(C3CCC(=O)N(C)C3=O)C4=O)CC2)=N1] 1-(7-cyclopentylpyrazolo[1,5-a]pyrimidin-6-yl)-3-[5-methyl-6-[5-[5-[4-[2-(1-methyl- 2,6-dioxo-3-piperidyl)-1-oxo-isoindolin-5-yl]piperazin-1-yl]pentyl]-1,2,4-oxadiazol-3- yl]-3-pyridyl]urea |
| 65 | 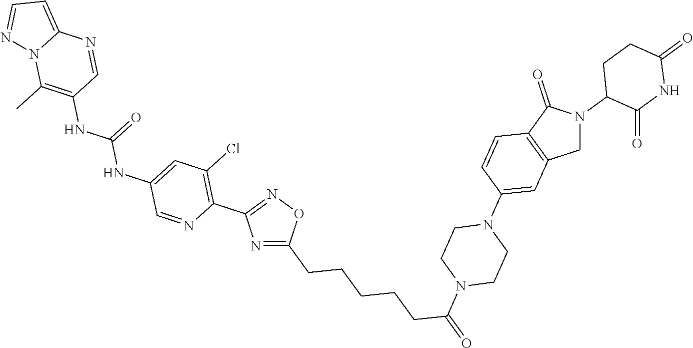 [SMILES: Cc1c(NC(=O)Nc2cnc(-c3noc(CCCCCC(=O)N4CCN(c5ccc6c(c5)CN(C5CCC(=O)NC5=O)C6=O)CC4)n3)c(Cl)c2)cnc2ccnn12] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-methylpyrazolo[1,5- a]pyrimidin-6-yl)urea |
| 66 | 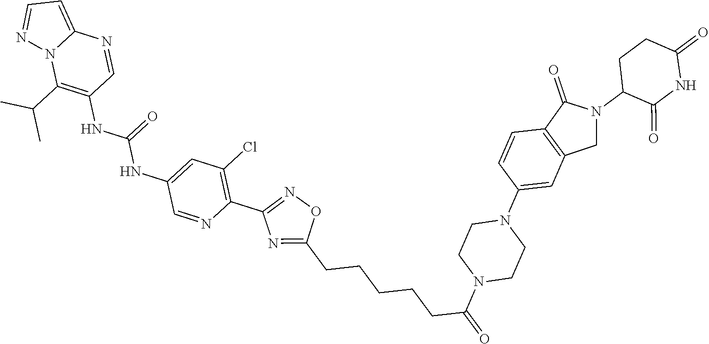 [SMILES: CC(C)c1c(NC(=O)Nc2cnc(-c3noc(CCCCCC(=O)N4CCN(c5ccc6c(c5)CN(C5CCC(=O)NC5=O)C6=O)CC4)n3)c(Cl)c2)cnc2ccnn12] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-isopropylpyrazolo[1,5- a]pyrimidin-6-yl)urea |
| 67 | 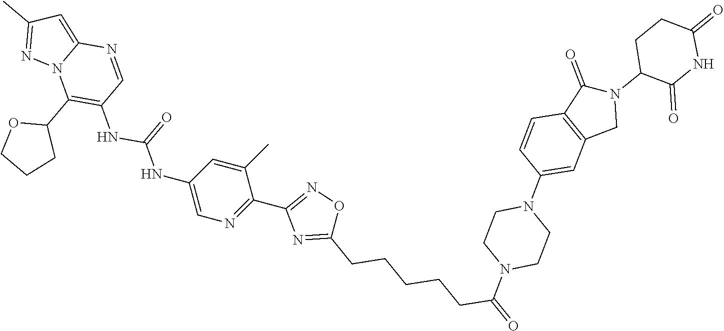 [SMILES: Cc1cc2ncc(NC(=O)Nc3cnc(-c4noc(CCCCCC(=O)N5CCN(c6ccc7c(c6)CN(C6CCC(=O)NC6=O)C7=O)CC5)n4)c(C)c3)c(C3CCCO3)n2n1] 1-[6-[5-[6-[4-[2-(2,6-dioxo-3-piperidyl)-1-oxo-isoindolin-5-yl]piperazin-1-yl]-6-oxo- hexyl]-1,2,4-oxadiazol-3-yl]-5-methyl-3-pyridyl]-3-(2-methyl-7-tetrahydrofuran-2-yl- pyrazolo[1,5-a]pyrimidin-6-yl)urea |
| 68 | 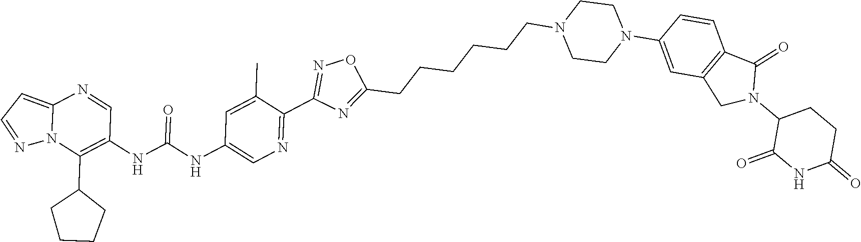 [SMILES: Cc1cc(NC(=O)Nc2cnc3ccnn3c2C2CCCC2)cnc1-c1noc(CCCCCCN2CCN(c3ccc4c(c3)CN(C3CCC(=O)NC3=O)C4=O)CC2)n1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-methyl- 3-pyridyl]urea |
| 69 | 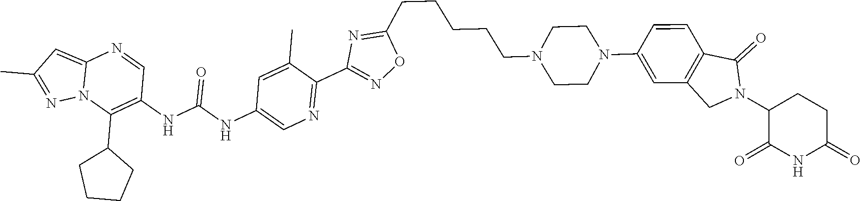 [SMILES: Cc1cc2ncc(NC(=O)Nc3cnc(-c4noc(CCCCCN5CCN(c6ccc7c(c6)CN(C6CCC(=O)NC6=O)C7=O)CC5)n4)c(C)c3)c(C3CCCC3)n2n1] 1-(7-cyclopentyl-2-methyl-pyrazolo[1,5-a]pyrimidin-6-yl)-3-[6-[5-[5-[4-[2-(2,6-dioxo- 3-piperidyl)-1-oxo-isoindolin-5-yl]piperazin-1-yl]pentyl]-1,2,4-oxadiazol-3-yl]-5- methyl-3-pyridyl]urea |
| 70 | 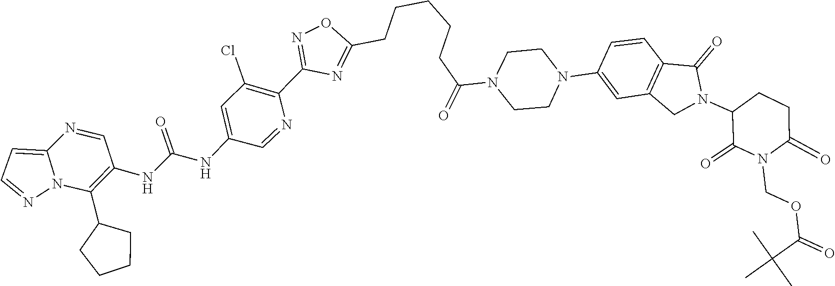 [SMILES: CC(C)(C)C(=O)OCN1C(=O)CCC(N2Cc3cc(N4CCN(C(=O)CCCCCc5nc(-c6ncc(NC(=O)Nc7cnc8ccnn8c7C7CCCC7)cc6Cl)no5)CC4)ccc3C2=O)C1=O] [3-[5-[4-[6-[3-[3-chloro-5-[(7-cyclopentylpyrazolo[1,5-a]pyrimidin-6- yl)carbamoylamino]-2-pyridyl]-1,2,4-oxadiazol-5-yl]hexanoyl]piperazin-1-yl]-1-oxo- isoindolin-2-yl]-2,6-dioxo-1-piperidyl]methyl 2,2-dimethylpropanoate |
| 71 | 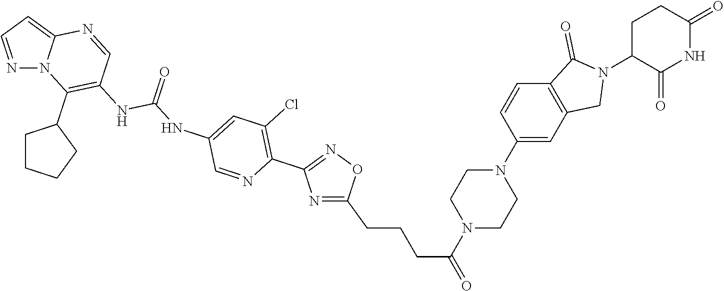 [SMILES: O=C1CCC(N2Cc3cc(N4CCN(C(=O)CCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]-4-oxo-butyl]-1,2,4-oxadiazol-3-yl]-3-pyridyl]-3-(7-cyclopentylpyrazolo[1,5- a]pyrimidin-6-yl)urea |
| 72 | 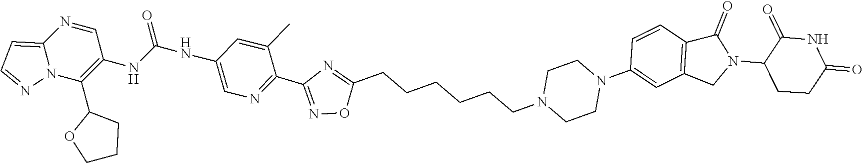 [SMILES: Cc1cc(NC(=O)Nc2cnc3ccnn3c2C2CCCO2)cnc1-c1noc(CCCCCCN2CCN(c3ccc4c(c3)CN(C3CCC(=O)NC3=O)C4=O)CC2)n1] 1-[6-[5-[6-[4-[2-(2,6-dioxo-3-piperidyl)-1-oxo-isoindolin-5-yl]piperazin-1-yl]hexyl]- 1,2,4-oxadiazol-3-yl]-5-methyl-3-pyridyl]-3-(7-tetrahydrofuran-2-ylpyrazolo[1,5- a]pyrimidin-6-yl)urea |
| 73 | 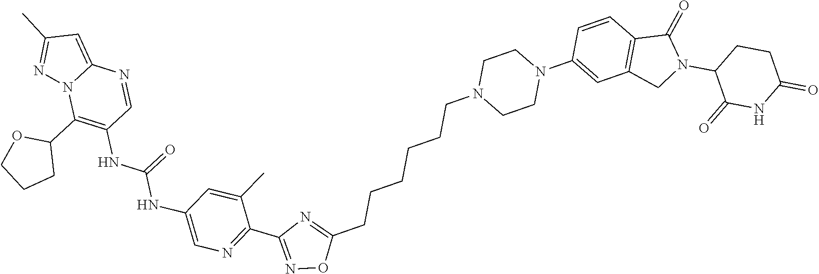 [SMILES: Cc1cc2ncc(NC(=O)Nc3cnc(-c4noc(CCCCCCN5CCN(c6ccc7c(c6)CN(C6CCC(=O)NC6=O)C7=O)CC5)n4)c(C)c3)c(C3CCCO3)n2n1] 1-[6-[5-[6-[4-[2-(2,6-dioxo-3-piperidyl)-1-oxo-isoindolin-5-yl]piperazin-1-yl]hexyl]- 1,2,4-oxadiazol-3-yl]-5-methyl-3-pyridyl]-3-(2-methyl-7-tetrahydrofuran-2-yl- pyrazolo[1,5-a]pyrimidin-6-yl)urea |
| 74 | 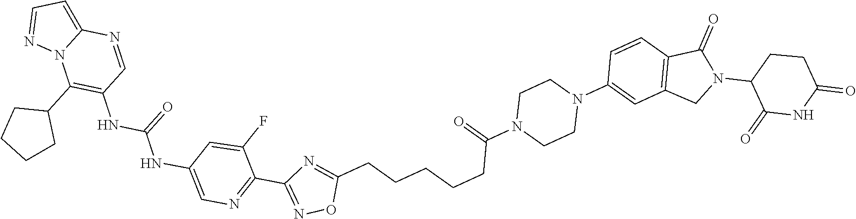 [SMILES: O=C1CCC(N2Cc3cc(N4CCN(C(=O)CCCCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- fluoro-3-pyridyl]urea |
| 75 | 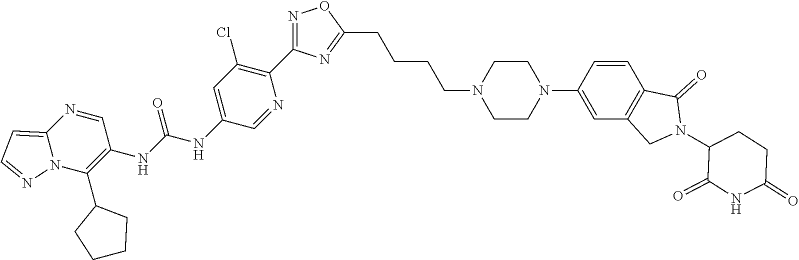 [SMILES: O=C1CCC(N2Cc3cc(N4CCN(CCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]butyl]-1,2,4-oxadiazol-3-yl]-3-pyridyl]-3-(7-cyclopentylpyrazolo[1,5-a]pyrimidin-6- yl)urea |
| 76 | 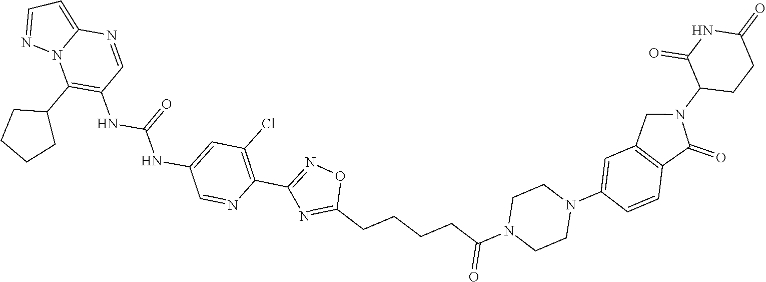 [SMILES: O=C1CCC(N2Cc3cc(N4CCN(C(=O)CCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]-5-oxo-pentyl]-1,2,4-oxadiazol-3-yl]-3-pyridyl]-3-(7-cyclopentylpyrazolo[1,5- a]pyrimidin-6-yl)urea |
| 77 | 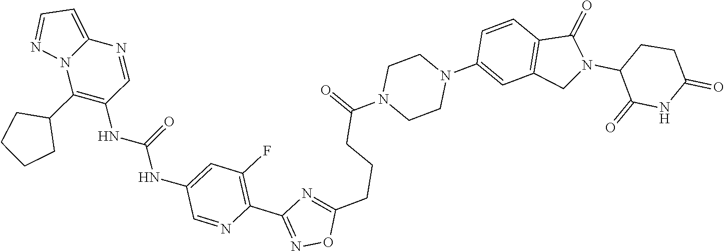 [SMILES: O=C1CCC(N2Cc3cc(N4CCN(C(=O)CCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- fluoro-3-pyridyl]urea |
| 78 | 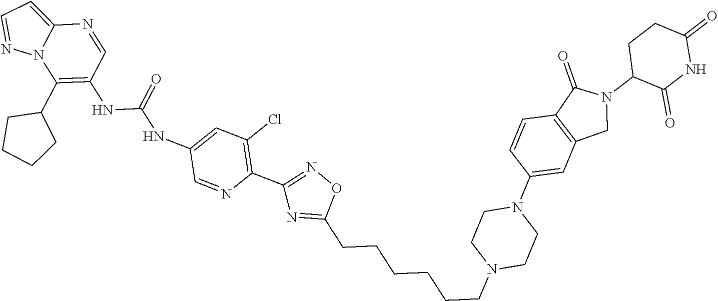 [SMILES: O=C1CCC(N2Cc3cc(N4CCN(CCCCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-cyclopentylpyrazolo[1,5-a]pyrimidin- 6-yl)urea |
| 79 | 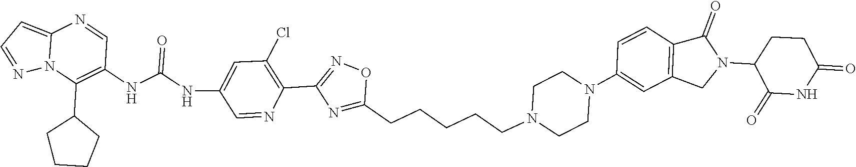 [SMILES: O=C1CCC(N2Cc3cc(N4CCN(CCCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]pentyl]-1,2,4-oxadiazol-3-yl]-3-pyridyl]-3-(7-cyclopentylpyrazolo[1,5-a]pyrimidin- 6-yl)urea |
| 80 | 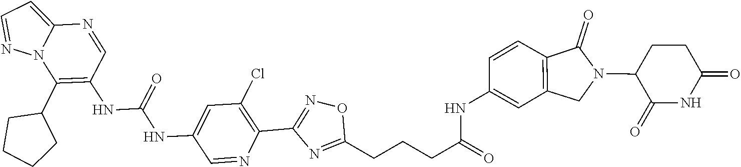 [SMILES: O=C1CCC(N2Cc3cc(NC(=O)CCCc4nc(-c5ncc(NC(=O)Nc6cnc7ccnn7c6C6CCCC6)cc5Cl)no4)ccc3C2=O)C(=O)N1] 4-[3-[3-chloro-5-[(7-cyclopentylpyrazolo[1,5-a]pyrimidin-6-yl)carbamoylamino]-2- pyridyl]-1,2,4-oxadiazol-5-yl]-N-[2-(2,6-dioxo-3-piperidyl)-1-oxo-isoindolin-5- yl]butanamide |
| 81 | 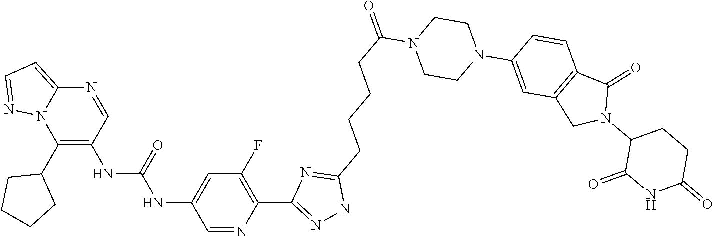 [SMILES: O=C1CCC(N2Cc3cc(N4CCN(C(=O)CCCCc5nc(-c6ncc(NC(=O)Nc7cnc8ccnn8c7C7CCCC7)cc6F)n[nH]5)CC4)ccc3C2=O)C(=O)N1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- fluoro-3-pyridyl]urea |
| 82 | 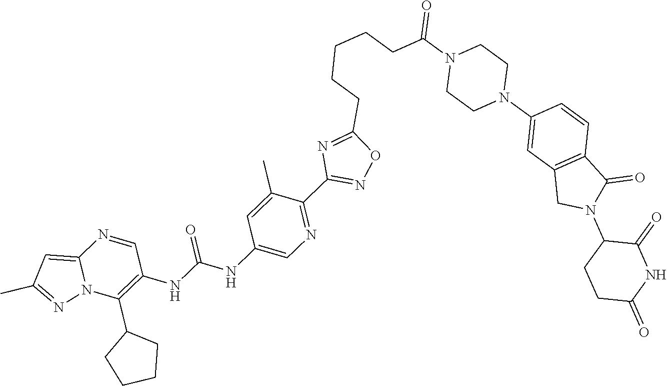 [SMILES: Cc1cc2ncc(NC(=O)Nc3cnc(-c4noc(CCCCCC(=O)N5CCN(c6ccc7c(c6)CN(C6CCC(=O)NC6=O)C7=O)CC5)n4)c(C)c3)c(C3CCCC3)n2n1] 1-(7-cyclopentyl-2-methyl-pyrazolo[1,5-a]pyrimidin-6-yl)-3-[6-[5-[6-[4-[2-(2,6-dioxo- 3-piperidyl)-1-oxo-isoindolin-5-yl]piperazin-1-yl]-6-oxo-hexyl]-1,2,4-oxadiazol-3-yl]- 5-methyl-3-pyridyl]urea |
| 83 | 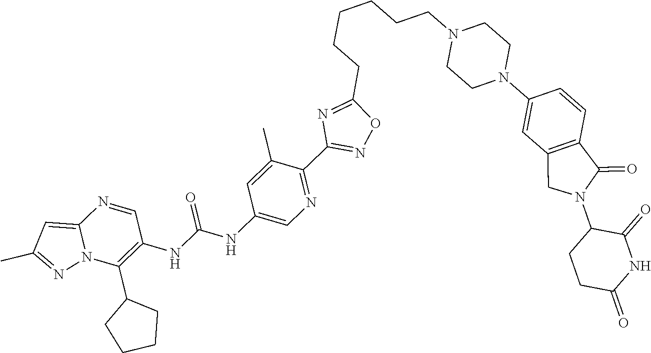 [SMILES: Cc1cc2ncc(NC(=O)Nc3cnc(-c4noc(CCCCCCN5CCN(c6ccc7c(c6)CN(C6CCC(=O)NC6=O)C7=O)CC5)n4)c(C)c3)c(C3CCCC3)n2n1] 1-(7-cyclopentyl-2-methyl-pyrazolo[1,5-a]pyrimidin-6-yl)-3-[6-[5-[6-[4-[2-(2,6-dioxo- 3-piperidyl)-1-oxo-isoindolin-5-yl]piperazin-1-yl]hexyl]-1,2,4-oxadiazol-3-yl]-5- methyl-3-pyridyl]urea |
| 84 | 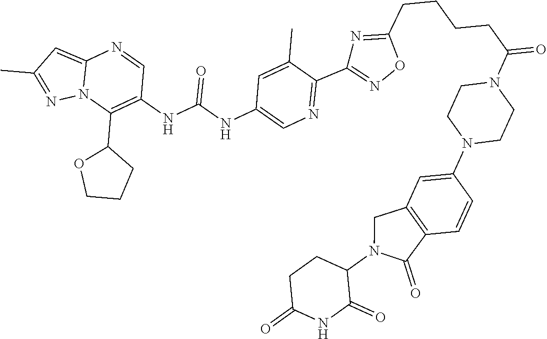 [SMILES: Cc1cc2ncc(NC(=O)Nc3cnc(-c4noc(CCCCC(=O)N5CCN(c6ccc7c(c6)CN(C6CCC(=O)NC6=O)C7=O)CC5)n4)c(C)c3)c(C3CCCO3)n2n1] 1-[6-[5-[5-[4-[2-(2,6-dioxo-3-piperidyl)-1-oxo-isoindolin-5-yl]piperazin-1-yl]-5-oxo- pentyl]-1,2,4-oxadiazol-3-yl]-5-methyl-3-pyridyl]-3-(2-methyl-7-tetrahydrofuran-2-yl- pyrazolo[1,5-a]pyrimidin-6-yl)urea |
| 85 | 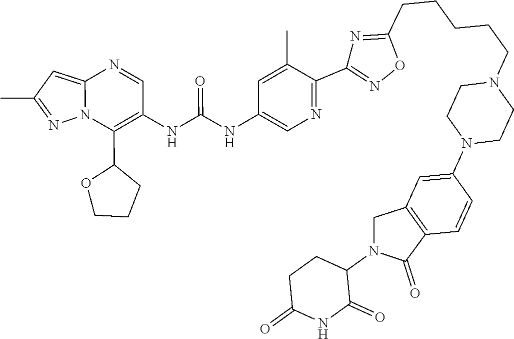 [SMILES: Cc1cc2ncc(NC(=O)Nc3cnc(-c4noc(CCCCCN5CCN(c6ccc7c(c6)CN(C6CCC(=O)NC6=O)C7=O)CC5)n4)c(C)c3)c(C3CCCO3)n2n1] 1-[6-[5-[5-[4-[2-(2,6-dioxo-3-piperidyl)-1-oxo-isoindolin-5-yl]piperazin-1-yl]pentyl]- 1,2,4-oxadiazol-3-yl]-5-methyl-3-pyridyl]-3-(2-methyl-7-tetrahydrofuran-2-yl- pyrazolo[1,5-a]pyrimidin-6-yl)urea |
| 86 | 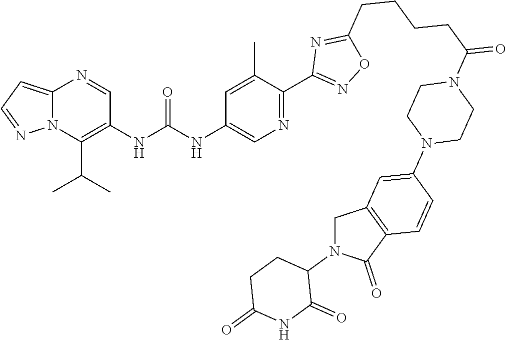 [SMILES: Cc1cc(NC(=O)Nc2cnc3ccnn3c2C(C)C)cnc1-c1noc(CCCCC(=O)N2CCN(c3ccc4c(c3)CN(C3CCC(=O)NC3=O)C4=O)CC2)n1] 1-[6-[5-[5-[4-[2-(2,6-dioxo-3-piperidyl)-1-oxo-isoindolin-5-yl]piperazin-1-yl]-5-oxo- pentyl]-1,2,4-oxadiazol-3-yl]-5-methyl-3-pyridyl]-3-(7-isopropylpyrazolo[1,5- a]pyrimidin-6-yl)urea |
| 87 | 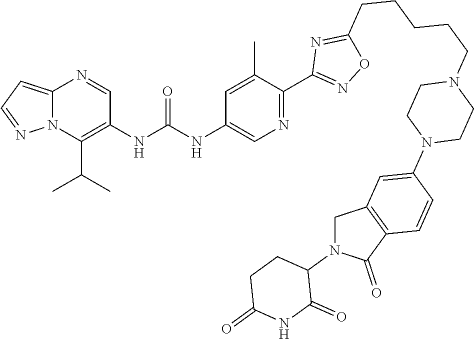 [SMILES: Cc1cc(NC(=O)Nc2cnc3ccnn3c2C(C)C)cnc1-c1noc(CCCCCN2CCN(c3ccc4c(c3)CN(C3CCC(=O)NC3=O)C4=O)CC2)n1] 1-[6-[5-[5-[4-[2-(2,6-dioxo-3-piperidyl)-1-oxo-isoindolin-5-yl]piperazin-1-yl]pentyl]- 1,2,4-oxadiazol-3-yl]-5-methyl-3-pyridyl]-3-(7-isopropylpyrazolo[1,5-a]pyrimidin-6- yl)urea |
| 88 | 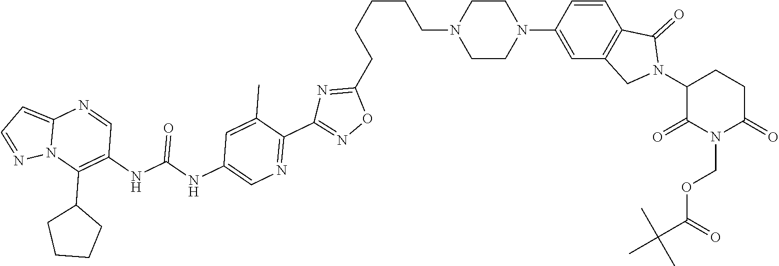 [SMILES: Cc1cc(NC(=O)Nc2cnc3ccnn3c2C2CCCC2)cnc1-c1noc(CCCCCN2CCN(c3ccc4c(c3)CN(C3CCC(=O)N(COC(=O)C(C)(C)C)C3=O)C4=O)CC2)n1] [3-[5-[4-[5-[3-[5-[(7-cyclopentylpyrazolo[1,5-a]pyrimidin-6-yl)carbamoylamino]-3- methyl-2-pyridyl]-1,2,4-oxadiazol-5-yl]pentyl]piperazin-1-yl]-1-oxo-isoindolin-2-yl]- 2,6-dioxo-1-piperidyl]methyl 2,2-dimethylpropanoate |
| 89 | 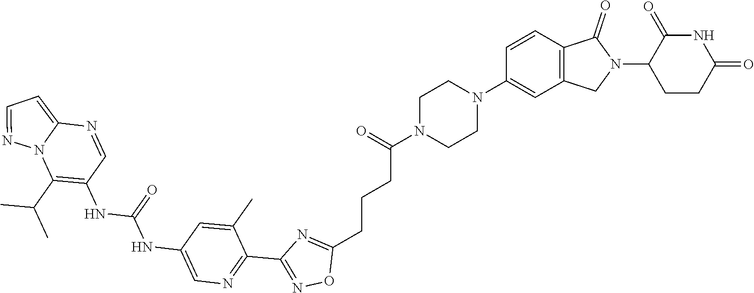 [SMILES: Cc1cc(NC(=O)Nc2cnc3ccnn3c2C(C)C)cnc1-c1noc(CCCC(=O)N2CCN(c3ccc4c(c3)CN(C3CCC(=O)NC3=O)C4=O)CC2)n1] 1-[6-[5-[4-[4-[2-(2,6-dioxo-3-piperidyl)-1-oxo-isoindolin-5-yl]piperazin-1-yl]-4-oxo- butyl]-1,2,4-oxadiazol-3-yl]-5-methyl-3-pyridyl]-3-(7-isopropylpyrazolo[1,5- a]pyrimidin-6-yl)urea |
| 90 | 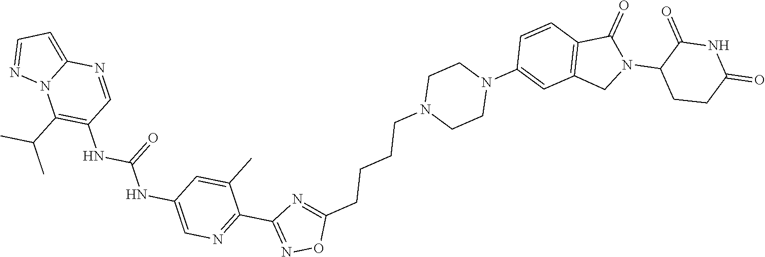 [SMILES: Cc1cc(NC(=O)Nc2cnc3ccnn3c2C(C)C)cnc1-c1noc(CCCCN2CCN(c3ccc4c(c3)CN(C3CCC(=O)NC3=O)C4=O)CC2)n1] 1-[6-[5-[4-[4-[2-(2,6-dioxo-3-piperidyl)-1-oxo-isoindolin-5-yl]piperazin-1-yl]butyl]- 1,2,4-oxadiazol-3-yl]-5-methyl-3-pyridyl]-3-(7-isopropylpyrazolo[1,5-a]pyrimidin-6- yl)urea |
| 91 | 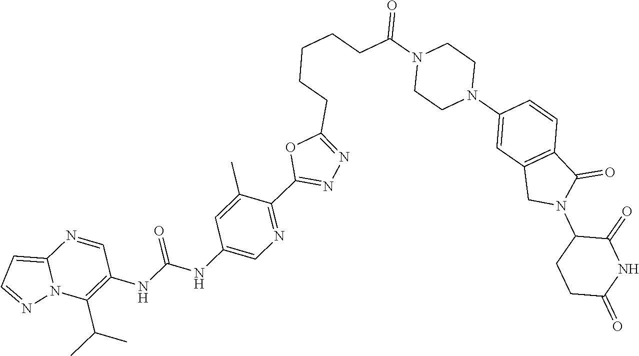 [SMILES: Cc1cc(NC(=O)Nc2cnc3ccnn3c2C(C)C)cnc1-c1nnc(CCCCCC(=O)N2CCN(c3ccc4c(c3)CN(C3CCC(=O)NC3=O)C4=O)CC2)o1] 1-[6-[5-[6-[4-[2-(2,6-dioxo-3-piperidyl)-1-oxo-isoindolin-5-yl]piperazin-1-yl]-6-oxo- hexyl]-1,2,4-oxadiazol-3-yl]-5-methyl-3-pyridyl]-3-(7-isopropylpyrazolo[1,5- a]pyrimidin-6-yl)urea |
| 92 | 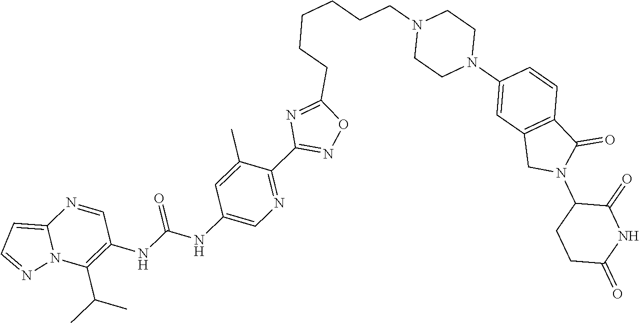 [SMILES: Cc1cc(NC(=O)Nc2cnc3ccnn3c2C(C)C)cnc1-c1noc(CCCCCCN2CCN(c3ccc4c(c3)CN(C3CCC(=O)NC3=O)C4=O)CC2)n1] 1-[6-[5-[6-[4-[2-(2,6-dioxo-3-piperidyl)-1-oxo-isoindolin-5-yl]piperazin-1-yl]hexyl]- 1,2,4-oxadiazol-3-yl]-5-methyl-3-pyridyl]-3-(7-isopropylpyrazolo[1,5-a]pyrimidin-6- yl)urea |
| 93 | 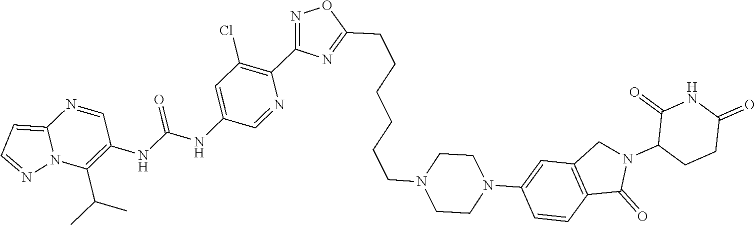 [SMILES: CC(C)c1c(NC(=O)Nc2cnc(-c3noc(CCCCCCN4CCN(c5ccc6c(c5)CN(C5CCC(=O)NC5=O)C6=O)CC4)n3)c(Cl)c2)cnc2ccnn12] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-isopropylpyrazolo[1,5-a]pyrimidin-6- yl)urea |
| 94 | 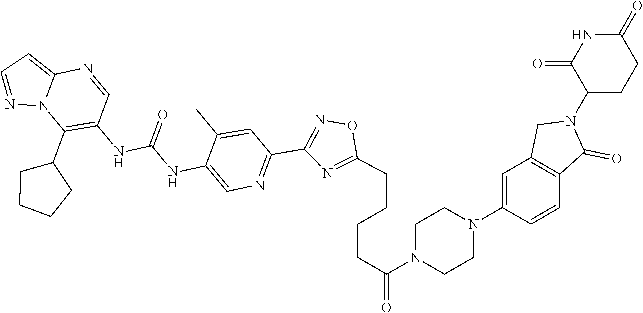 [SMILES: Cc1cc(-c2noc(CCCCC(=O)N3CCN(c4ccc5c(c4)CN(C4CCC(=O)NC4=O)C5=O)CC3)n2)ncc1NC(=O)Nc1cnc2ccnn2c1C1CCCC1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-4- methyl-3-pyridyl]urea |
| 95 | 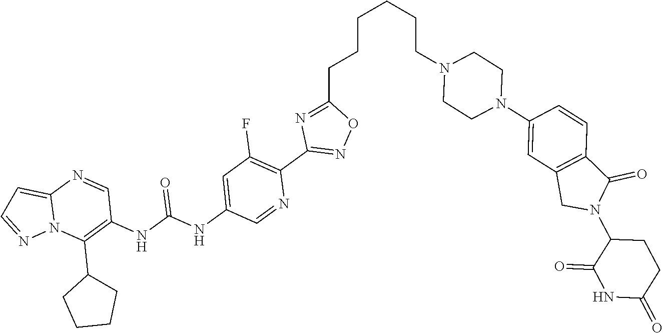 [SMILES: O=C1CCC(N2Cc3cc(N4CCN(CCCCCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-fluoro- 3-pyridyl]urea |
| 96 | 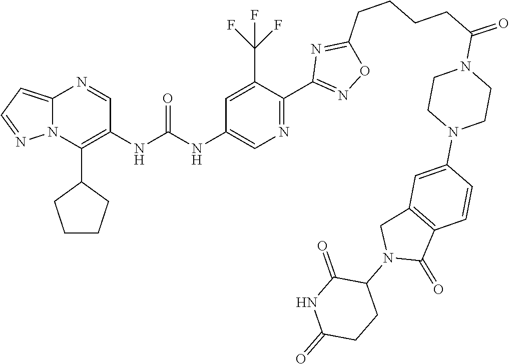 [SMILES: O=C1CCC(N2Cc3cc(N4CCN(C(=O)CCCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- (trifluoromethyl)-3-pyridyl]urea |
| 97 | 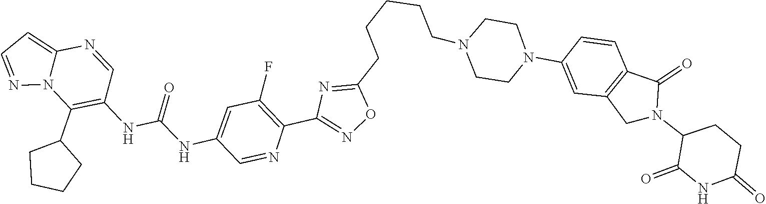 [SMILES: O=C1CCC(N2Cc3cc(N4CCN(CCCCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5-fluoro- 3-pyridyl]urea |
| 98 | 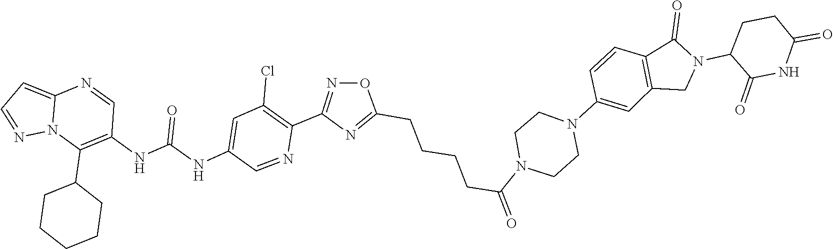 [SMILES: O=C1CCC(N2Cc3cc(N4CCN(C(=O)CCCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]-5-oxo-pentyl]-1,2,4-oxadiazol-3-yl]-3-pyridyl]-3-(7-cyclohexylpyrazolo[1,5- a]pyrimidin-6-yl)urea |
| 99 | 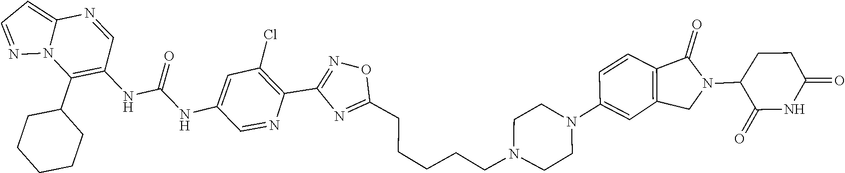 [SMILES: O=C1CCC(N2Cc3cc(N4CCN(CCCCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]pentyl]-1,2,4-oxadiazol-3-yl]-3-pyridyl]-3-(7-cyclohexylpyrazolo[1,5-a]pyrimidin- 6-yl)urea |
| 100 | 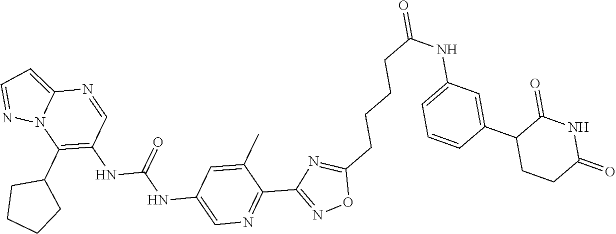 [SMILES: Cc1cc(NC(=O)Nc2cnc3ccnn3c2C2CCCC2)cnc1-c1noc(CCCCC(=O)Nc2cccc(C3CCC(=O)NC3=O)c2)n1] 5-[3-[5-[(7-cyclopentylpyrazolo[1,5-a]pyrimidin-6-yl)carbamoylamino]-3-methyl-2- pyridyl]-1,2,4-oxadiazol-5-yl]-N-[3-(2,6-dioxo-3-piperidyl)phenyl]pentanamide |
| 101 | 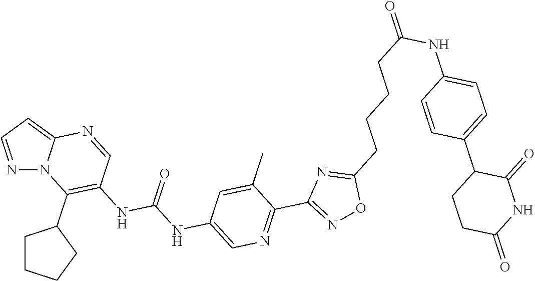 [SMILES: Cc1cc(NC(=O)Nc2cnc3ccnn3c2C2CCCC2)cnc1-c1noc(CCCCC(=O)Nc2ccc(C3CCC(=O)NC3=O)cc2)n1] 5-[3-[5-[(7-cyclopentylpyrazolo[1,5-a]pyrimidin-6-yl)carbamoylamino]-3-methyl-2- pyridyl]-1,2,4-oxadiazol-5-yl]-N-[4-(2,6-dioxo-3-piperidyl)phenyl]pentanamide |
| 102 | 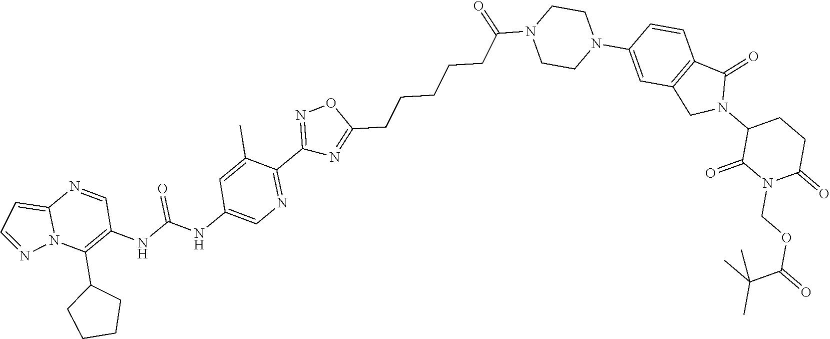 [SMILES: Cc1cc(NC(=O)Nc2cnc3ccnn3c2C2CCCC2)cnc1-c1noc(CCCCCC(=O)N2CCN(c3ccc4c(c3)CN(C3CCC(=O)N(COC(=O)C(C)(C)C)C3=O)C4=O)CC2)n1] [3-[5-[4-[6-[3-[5-[(7-cyclopentylpyrazolo[1,5-a]pyrimidin-6-yl)carbamoylamino]-3- methyl-2-pyridyl]-1,2,4-oxadiazol-5-yl]hexanoyl]piperazin-1-yl]-1-oxo-isoindolin-2- yl]-2,6-dioxo-1-piperidyl]methyl 2,2-dimethylpropanoate |
| 103 | 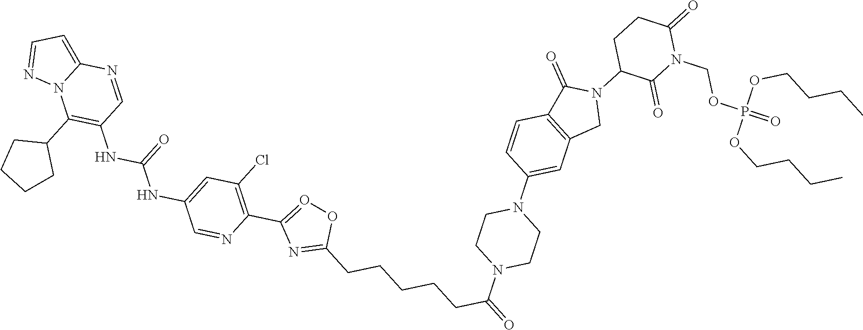 dibutyl [3-[5-[4-[6-[3-[3-chloro-5-[(7-cyclopentylpyrazolo[1,5-a]pyrimidin-6- yl)carbamoylamino]-2-pyridyl]-1,2,4-oxadiazol-5-yl]hexanoyl]piperazin-1-yl]-1-oxo- isoindolin-2-yl]-2,6-dioxo-1-piperidyl]methyl phosphate |
| 104 | 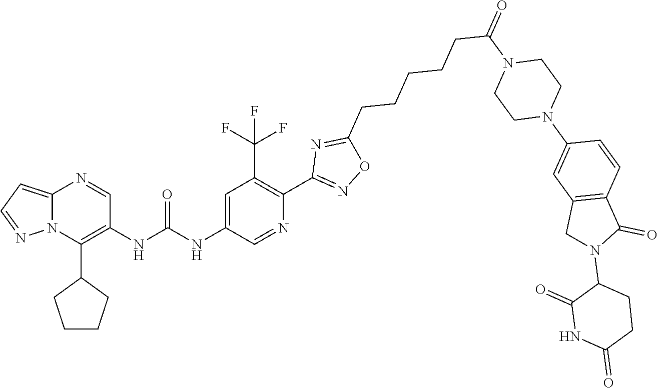 [SMILES: O=C1CCC(N2Cc3cc(N4CCN(C(=O)CCCCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- (trifluoromethyl)-3-pyridyl]urea |
| 105 | 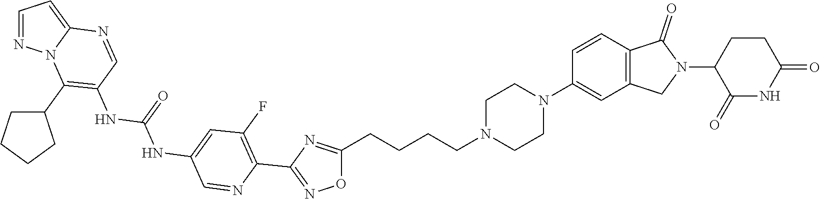 [SMILES: O=C1CCC(N2Cc3cc(N4CCN(CCCCc5nc(-c6ncc(NC(=O)Nc7cnc8ccnn8c7C7CCCC7)cc6F)no5)CC4)ccc3C2=O)C(=O)N1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butyl]-1,2,4-oxadiazol-3-yl]-5-fluoro- 3-pyridyl]urea |
| 106 | 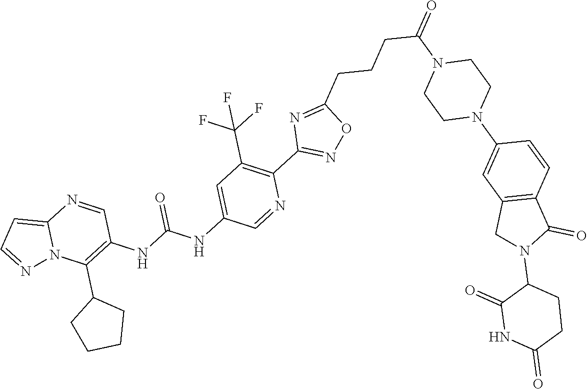 [SMILES: O=C1CCC(N2Cc3cc(N4CCN(C(=O)CCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- (trifluoromethyl)-3-pyridyl]urea |
| 107 | 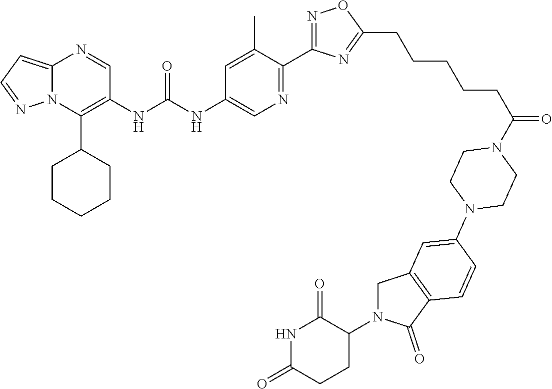 [SMILES: Cc1cc(NC(=O)Nc2cnc3ccnn3c2C2CCCCC2)cnc1-c1noc(CCCCCC(=O)N2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 108 | 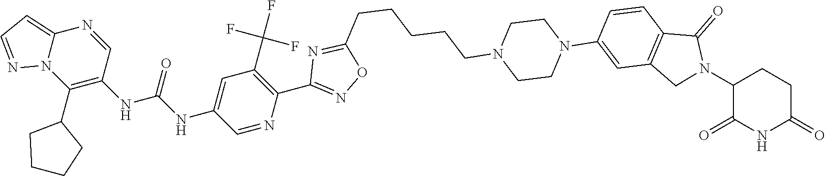 [SMILES: O=C1CCC(N2Cc3cc(N4CCN(CCCCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- (trifluoromethyl)-3-pyridyl]urea |
| 109 | 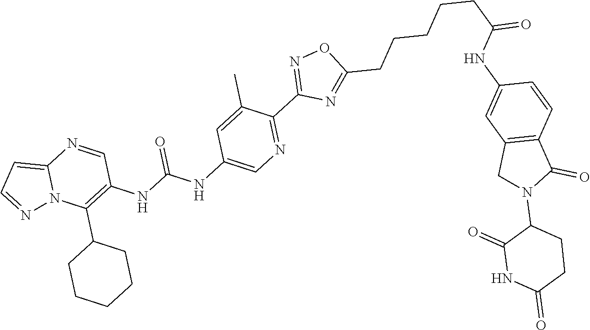 [SMILES: Cc1cc(NC(=O)Nc2cnc3ccnn3c2C2CCCCC2)cnc1-c1noc(CCCCCC(=O)Nc2ccc3c(c2)CN(C2CCC(=O)NC2=O)C3=O)n1] 6-[3-[5-[(7-cyclohexylpyrazolo[1,5-a]pyrimidin-6-yl)carbamoylamino]-3-methyl-2- pyridyl]-1,2,4-oxadiazol-5-yl]-N-[2-(2,6-dioxo-3-piperidyl)-1-oxo-isoindolin-5- yl]hexanamide |
| 110 | 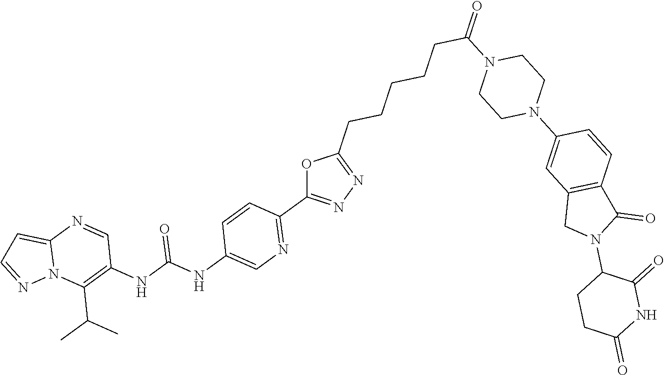 [SMILES: CC(C)c1c(NC(=O)Nc2ccc(-c3nnc(CCCCCC(=O)N4CCN(c5ccc6c(c5)CN(C5CCC(=O)NC5=O)C6=O)CC4)o3)nc2)cnc2ccnn12] 1-[6-[5-[6-[4-[2-(2,6-dioxo-3-piperidyl)-1-oxo-isoindolin-5-yl]piperazin-1-yl]-6-oxo- hexyl]-1,3,4-oxadiazol-2-yl]-3-pyridyl]-3-(7-isopropylpyrazolo[1,5-a]pyrimidin-6- yl)urea |
| 111 | 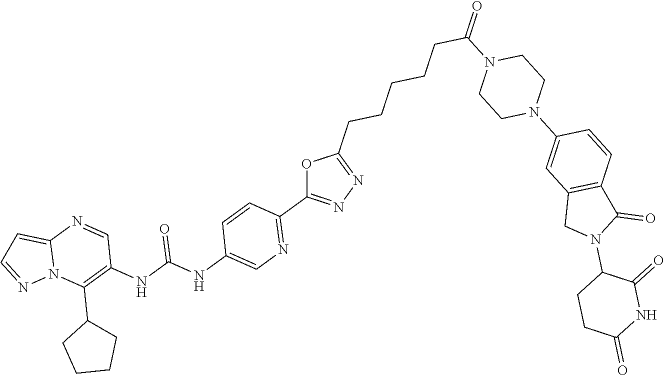 [SMILES: O=C1CCC(N2Cc3cc(N4CCN(C(=O)CCCCCc5nnc(-c6ccc(NC(=O)Nc7cnc8ccnn8c7C7CCCC7)cn6)o5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,3,4-oxadiazol-2-yl]-3- pyridyl]urea |
| 112 | 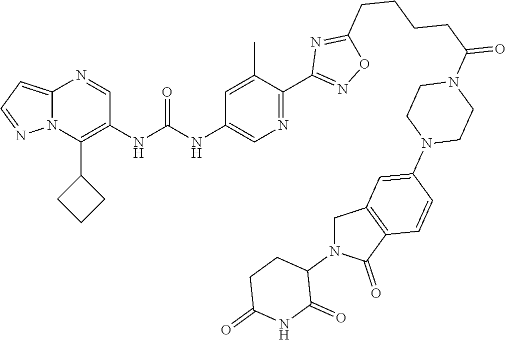 [SMILES: Cc1cc(NC(=O)Nc2cnc3ccnn3c2C2CCC2)cnc1-c1noc(CCCCC(=O)N2CCN(c3ccc4c(c3)CN(C3CCC(=O)NC3=O)C4=O)CC2)n1] 1-(7-cyclobutylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- methyl-3-pyridyl]urea |
| 113 | 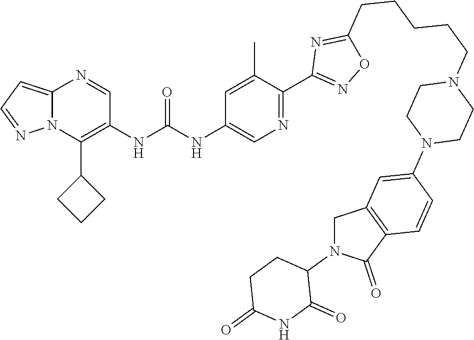 [SMILES: Cc1cc(NC(=O)Nc2cnc3ccnn3c2C2CCC2)cnc1-c1noc(CCCCCN2CCN(c3ccc4c(c3)CN(C3CCC(=O)NC3=O)C4=O)CC2)n1] 1-(7-cyclobutylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- methyl-3-pyridyl]urea |
| 114 | 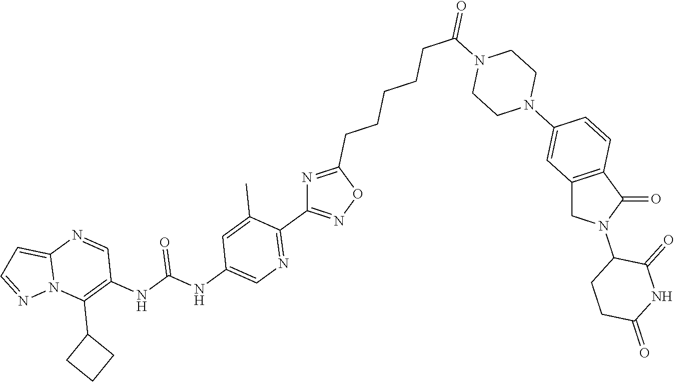 [SMILES: Cc1cc(NC(=O)Nc2cnc3ccnn3c2C2CCC2)cnc1-c1noc(CCCCCC(=O)N2CCN(c3ccc4c(c3)CN(C3CCC(=O)NC3=O)C4=O)CC2)n1] 1-(7-cyclobutylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 115 | 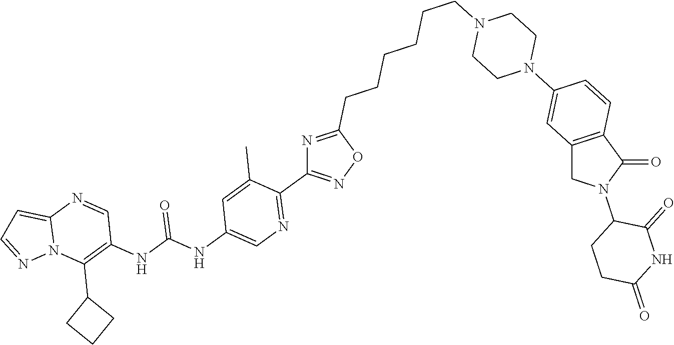 [SMILES: Cc1cc(NC(=O)Nc2cnc3ccnn3c2C2CCC2)cnc1-c1noc(CCCCCCN2CCN(c3ccc4c(c3)CN(C3CCC(=O)NC3=O)C4=O)CC2)n1] 1-(7-cyclobutylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-methyl- 3-pyridyl]urea |
| 116 | 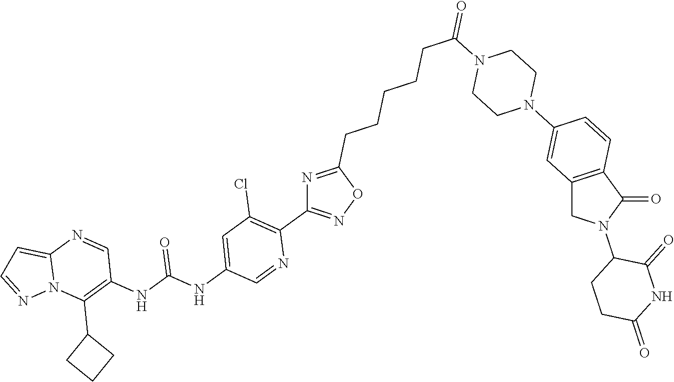 [SMILES: O=C1CCC(N2Cc3cc(N4CCN(C(=O)CCCCCc5nc(-c6ncc(NC(=O)Nc7cnc8ccnn8c7C7CCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-cyclobutylpyrazolo[1,5- a]pyrimidin-6-yl)urea |
| 117 | 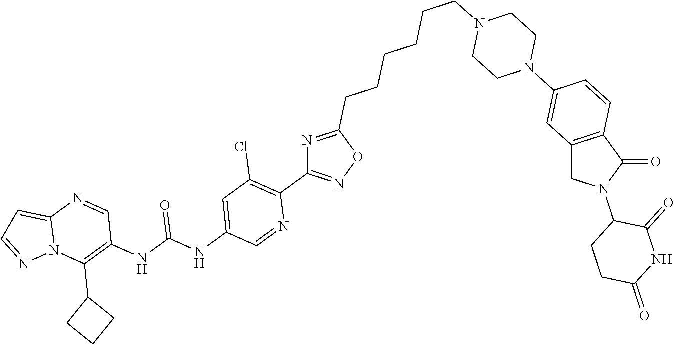 [SMILES: O=C1CCC(N2Cc3cc(N4CCN(CCCCCCc5nc(-c6ncc(NC(=O)Nc7cnc8ccnn8c7C7CCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-cyclobutylpyrazolo[1,5-a]pyrimidin-6- yl)urea |
| 118 | 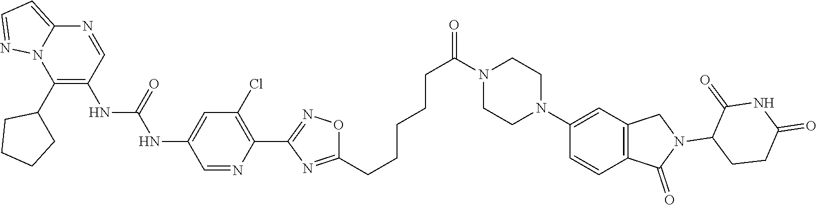 [SMILES: O=C1CCC(N2Cc3cc(N4CCN(C(=O)CCCCCc5nc(-c6ncc(NC(=O)Nc7cnc8ccnn8c7C7CCCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-cyclohexylpyrazolo[1,5- a]pyrimidin-6-yl)urea |
| 119 | 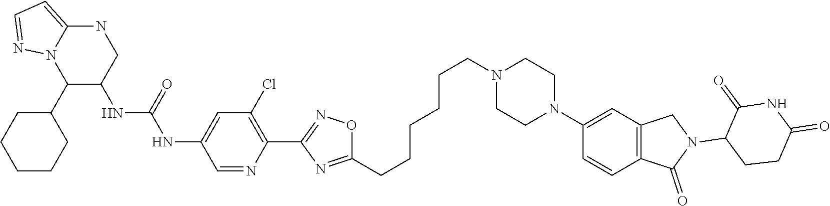 [SMILES: O=C1CCC(N2Cc3cc(N4CCN(CCCCCCc5nc(-c6ncc(NC(=O)NC7CNc8ccnn8C7C7CCCCC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-cyclohexylpyrazolo[1,5-a]pyrimidin-6- yl)urea |
| 120 | 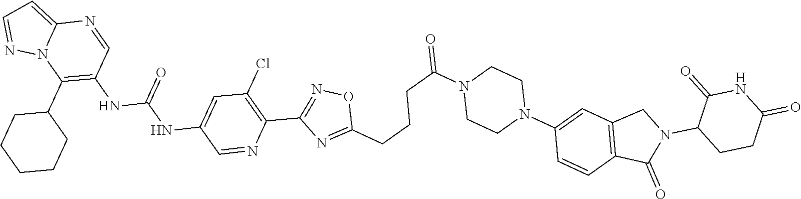 [SMILES: O=C1CCC(N2Cc3cc(N4CCN(C(=O)CCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]-4-oxo-butyl]-1,2,4-oxadiazol-3-yl]-3-pyridyl]-3-(7-cyclohexylpyrazolo[1,5- a]pyrimidin-6-yl)urea |
| 121 | 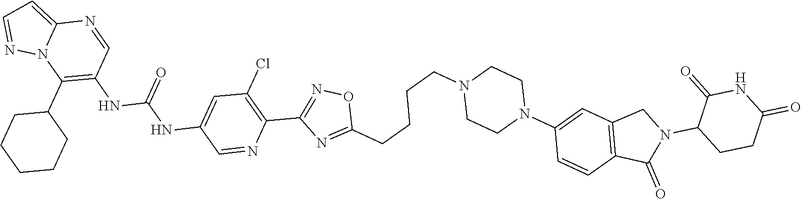 [SMILES: O=C1CCC(N2Cc3cc(N4CCN(CCCCc5nc(-c6ncc(NC(=O)Nc7cnc8ccnn8c7C7CCCCC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]butyl]-1,2,4-oxadiazol-3-yl]-3-pyridyl]-3-(7-cyclohexylpyrazolo[1,5-a]pyrimidin-6- yl)urea |
| 122 | 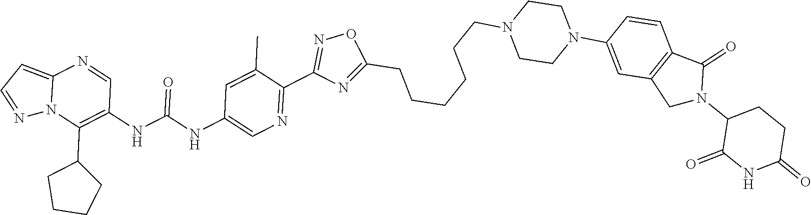 [SMILES: Cc1cc(NC(=O)Nc2cnc3ccnn3c2C2CCCC2)cnc1-c1noc(CCCCCCN2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-methyl- 3-pyridyl]urea |
| 123 | 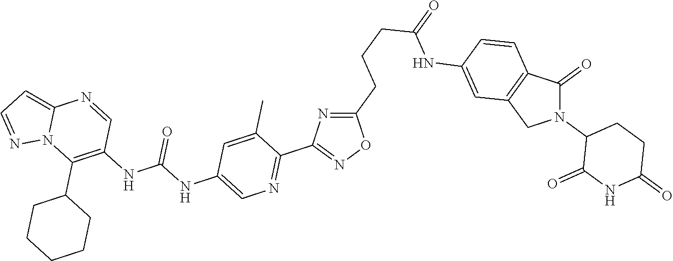 [SMILES: Cc1cc(NC(=O)Nc2cnc3ccnn3c2C2CCCCC2)cnc1-c1noc(CCCC(=O)Nc2ccc3c(c2)CN(C2CCC(=O)NC2=O)C3=O)n1] 4-[3-[5-[(7-cyclohexylpyrazolo[1,5-a]pyrimidin-6-yl)carbamoylamino]-3-methyl-2- pyridyl]-1,2,4-oxadiazol-5-yl]-N-[2-(2,6-dioxo-3-piperidyl)-1-oxo-isoindolin-5- yl]butanamide |
| 124 | 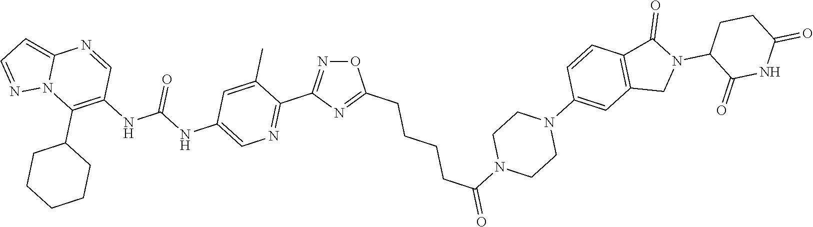 [SMILES: Cc1cc(NC(=O)Nc2cnc3ccnn3c2C2CCCCC2)cnc1-c1noc(CCCCC(=O)N2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- methyl-3-pyridyl]urea |
| 125 | 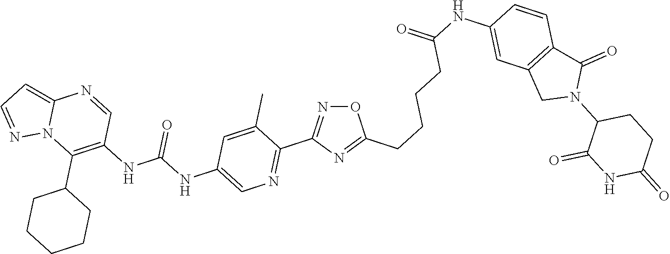 [SMILES: Cc1cc(NC(=O)Nc2cnc3ccnn3c2C2CCCCC2)cnc1-c1noc(CCCCC(=O)Nc2ccc3c(c2)CN(C2CCC(=O)NC2=O)C3=O)n1] 5-[3-[5-[(7-cyclohexylpyrazolo[1,5-a]pyrimidin-6-yl)carbamoylamino]-3-methyl-2- pyridyl]-1,2,4-oxadiazol-5-yl]-N-[2-(2,6-dioxo-3-piperidyl)-1-oxo-isoindolin-5- yl]pentanamide |
| 126 | 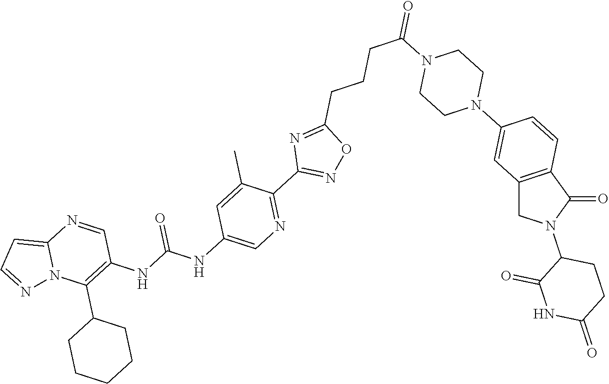 [SMILES: Cc1cc(NC(=O)Nc2cnc3ccnn3c2C2CCCCC2)cnc1-c1noc(CCCC(=O)N2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- methyl-3-pyridyl]urea |
| 127 | 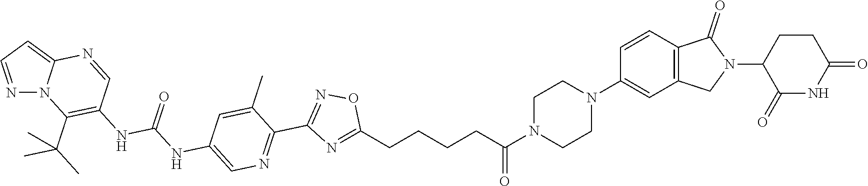 [SMILES: Cc1cc(NC(=O)Nc2cnc3ccnn3c2C(C)(C)C)cnc1-c1noc(CCCCC(=O)N2CCN(c3ccc4c(c3)CN(C3CCC(=O)NC3=O)C4=O)CC2)n1] 1-(7-tert-butylpyrazolo[1,5-a]pyrimidin-6-yl)-3-[6-[5-[5-[4-[2-(2,6-dioxo-3-piperidyl)- 1-oxo-isoindolin-5-yl]piperazin-1-yl]-5-oxo-pentyl]-1,2,4-oxadiazol-3-yl]-5-methyl-3- pyridyl]urea |
| 128 | 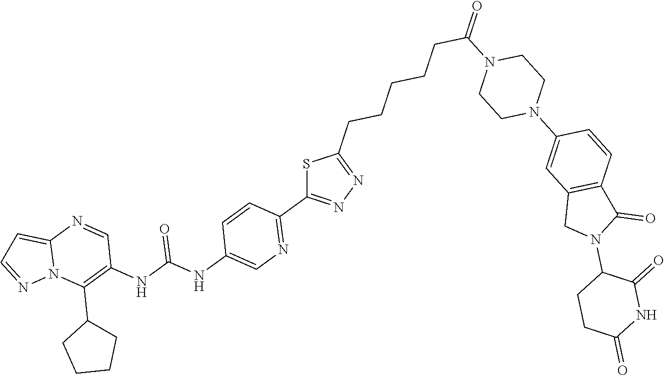 [SMILES: O=C1CCC(N2Cc3cc(N4CCN(C(=O)CCCCCc5nnc(-c6ccc(NC(=O)Nc7cnc8ccnn8c7C7CCCC7)cn6)s5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,3,4-thiadiazol-2-yl]-3- pyridyl]urea |
| 129 | 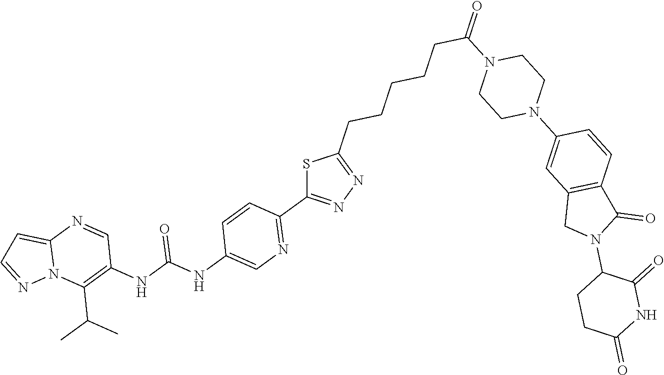 [SMILES: CC(C)c1c(NC(=O)Nc2ccc(-c3nnc(CCCCCC(=O)N4CCN(c5ccc6c(c5)CN(C5CCC(=O)NC5=O)C6=O)CC4)s3)nc2)cnc2ccnn12] 1-[6-[5-[6-[4-[2-(2,6-dioxo-3-piperidyl)-1-oxo-isoindolin-5-yl]piperazin-1-yl]-6-oxo- hexyl]-1,3,4-thiadiazol-2-yl]-3-pyridyl]-3-(7-isopropylpyrazolo[1,5-a]pyrimidin-6- yl)urea |
| 130 | 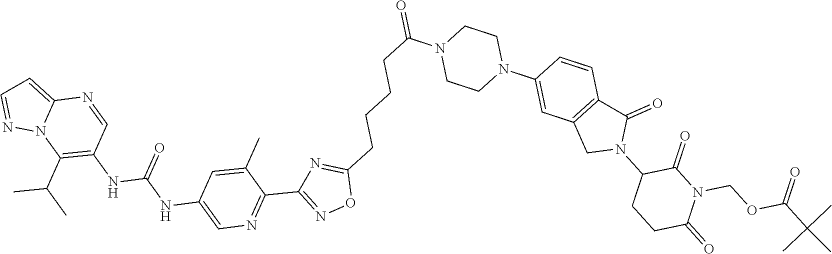 [SMILES: Cc1cc(NC(=O)Nc2cnc3ccnn3c2C(C)C)cnc1-c1noc(CCCCC(=O)N2CCN(c3ccc4c(c3)CN(C3CCC(=O)N(COC(=O)C(C)(C)C)C3=O)C4=O)CC2)n1] [3-[5-[4-[5-[3-[5-[(7-isopropylpyrazolo[1,5-a]pyrimidin-6-yl)carbamoylamino]-3- methyl-2-pyridyl]-1,2,4-oxadiazol-5-yl]pentanoyl]piperazin-1-yl]-1-oxo-isoindolin-2- yl]-2,6-dioxo-1-piperidyl]methyl 2,2-dimethylpropanoate |
| 131 | 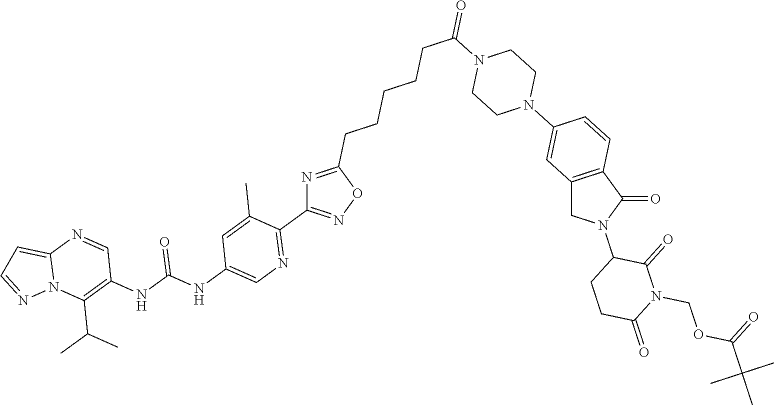 [SMILES: Cc1cc(NC(=O)Nc2cnc3ccnn3c2C(C)C)cnc1-c1noc(CCCCCC(=O)N2CCN(c3ccc4c(c3)CN(C3CCC(=O)N(COC(=O)C(C)(C)C)C3=O)C4=O)CC2)n1] [3-[5-[4-[6-[3-[5-[(7-isopropylpyrazolo[1,5-a]pyrimidin-6-yl)carbamoylamino]-3- methyl-2-pyridyl]-1,2,4-oxadiazol-5-yl]hexanoyl]piperazin-1-yl]-1-oxo-isoindolin-2- yl]-2,6-dioxo-1-piperidyl]methyl 2,2-dimethylpropanoate |
| 132 | 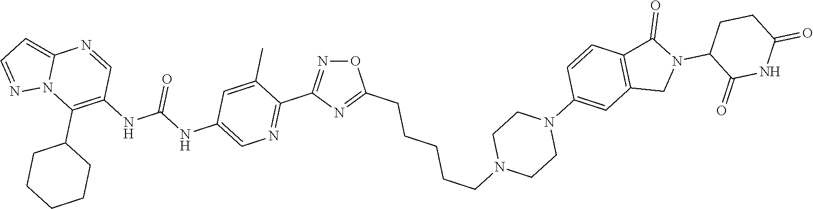 [SMILES: Cc1cc(NC(=O)Nc2cnc3ccnn3c2C2CCCCC2)cnc1-c1noc(CCCCCN2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- methyl-3-pyridyl]urea |
| 133 | 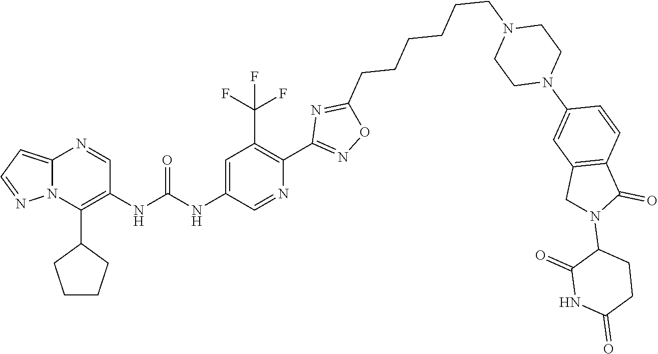 [SMILES: O=C1CCC(N2Cc3cc(N4CCN(CCCCCCc5nc(-c6ncc(NC(=O)Nc7cnc8ccnn8c7C7CCCC7)cc6C(F)(F)F)no5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5- (trifluoromethyl)-3-pyridyl]urea |
| 134 | 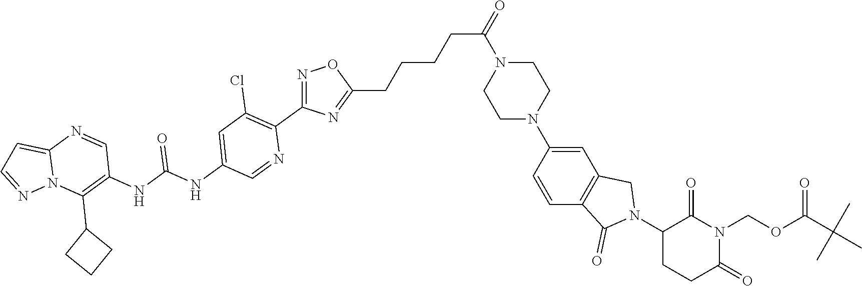 [SMILES: CC(C)(C)C(=O)OCN1C(=O)CCC(N2Cc3cc(N4CCN(C(=O)CCCCc5nc(-c6ncc(NC(=O)Nc7cnc8ccnn8c7C7CCC7)cc6Cl)no5)CC4)ccc3C2=O)C1=O] [3-[5-[4-[5-[3-[3-chloro-5-[(7-cyclopentylpyrazolo[1,5-a]pyrimidin-6- yl)carbamoylamino]-2-pyridyl]-1,2,4-oxadiazol-5-yl]pentanoyl]piperazin-1-yl]-1-oxo- isoindolin-2-yl]-2,6-dioxo-1-piperidyl]methyl 2,2-dimethylpropanoate |
| 135 | 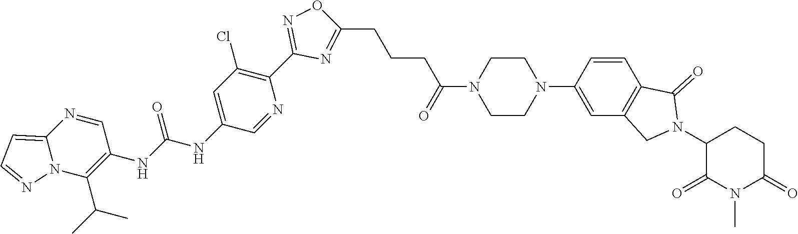 [SMILES: CC(C)c1c(NC(=O)Nc2cnc(-c3noc(CCCC(=O)N4CCN(c5ccc6c(c5)CN(C5CCC(=O)N(C)C5=O)C6=O)CC4)n3)c(Cl)c2)cnc2ccnn12] 1-[5-chloro-6-[5-[4-[4-[2-(1-methyl-2,6-dioxo-3-piperidyl)-1-oxo-isoindolin-5- yl]piperazin-1-yl]-4-oxo-butyl]-1,2,4-oxadiazol-3-yl]-3-pyridyl]-3-(7- isopropylpyrazolo[1,5-a]pyrimidin-6-yl)urea |
| 136 | 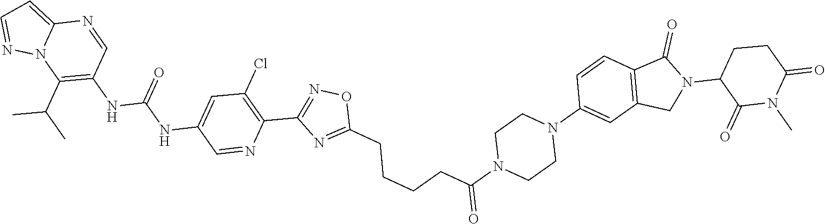 [SMILES: CC(C)c1c(NC(=O)Nc2cnc(-c3noc(CCCCC(=O)N4CCN(c5ccc6c(c5)CN(C5CCC(=O)N(C)C5=O)C6=O)CC4)n3)c(Cl)c2)cnc2ccnn12] 1-[5-chloro-6-[5-[5-[4-[2-(1-methyl-2,6-dioxo-3-piperidyl)-1-oxo-isoindolin-5- yl]piperazin-1-yl]-5-oxo-pentyl]-1,2,4-oxadiazol-3-yl]-3-pyridyl]-3-(7- isopropylpyrazolo[1,5-a]pyrimidin-6-yl)urea |
| 137 | 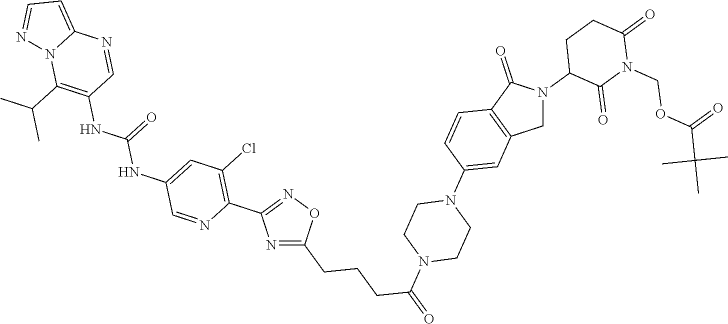 [SMILES: CC(C)c1c(NC(=O)Nc2cnc(-c3noc(CCCC(=O)N4CCN(c5ccc6c(c5)CN(C5CCC(=O)N(COC(=O)C(C)(C)C)C5=O)C6=O)CC4)n3)c(Cl)c2)cnc2ccnn12] [3-[5-[4-[4-[3-[3-chloro-5-[(7-isopropylpyrazolo[1,5-a]pyrimidin-6- yl)carbamoylamino]-2-pyridyl]-1,2,4-oxadiazol-5-yl]butanoyl]piperazin-1-yl]-1-oxo- isoindolin-2-yl]-2,6-dioxo-1-piperidyl]methyl 2,2-dimethylpropanoate |
| 138 | 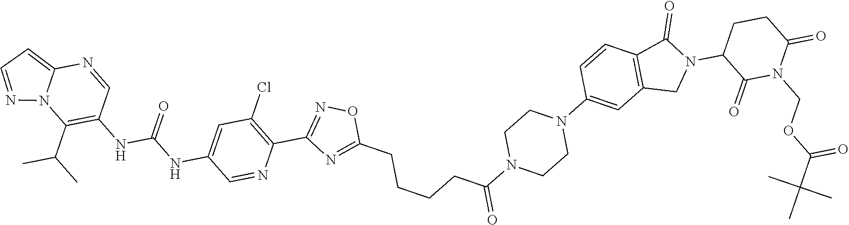 [SMILES: CC(C)c1c(NC(=O)Nc2cnc(-c3noc(CCCCC(=O)N4CCN(c5ccc6c(c5)CN(C5CCC(=O)N(COC(=O)C(C)(C)C)C5=O)C6=O)CC4)n3)c(Cl)c2)cnc2ccnn12] [3-[5-[4-[5-[3-[3-chloro-5-[(7-isopropylpyrazolo[1,5-a]pyrimidin-6- yl)carbamoylamino]-2-pyridyl]-1,2,4-oxadiazol-5-yl]pentanoyl]piperazin-1-yl]-1-oxo- isoindolin-2-yl]-2,6-dioxo-1-piperidyl]methyl 2,2-dimethylpropanoate |
| 139 | 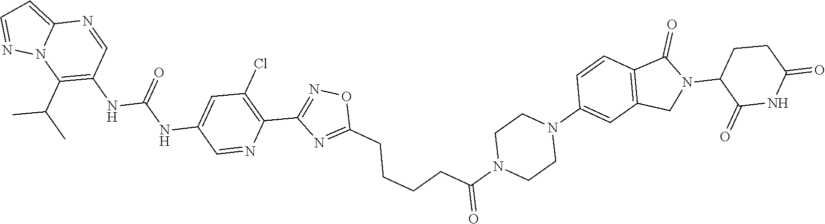 [SMILES: CC(C)c1c(NC(=O)Nc2cnc(-c3noc(CCCCC(=O)N4CCN(c5ccc6c(c5)CN(C5CCC(=O)NC5=O)C6=O)CC4)n3)c(Cl)c2)cnc2ccnn12] 1-[5-chloro-6-[5-[5-[4-[2-(2,6-dioxo-3-piperidyl)-1-oxo-isoindolin-5-yl]piperazin-1- yl]-5-oxo-pentyl]-1,2,4-oxadiazol-3-yl]-3-pyridyl]-3-(7-isopropylpyrazolo[1,5- a]pyrimidin-6-yl)urea |
| 140 | 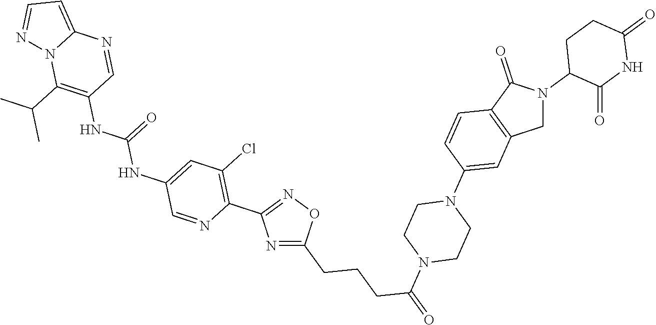 [SMILES: CC(C)c1c(NC(=O)Nc2cnc(-c3noc(CCCC(=O)N4CCN(c5ccc6c(c5)CN(C5CCC(=O)NC5=O)C6=O)CC4)n3)c(Cl)c2)cnc2ccnn12] 1-[5-chloro-6-[5-[4-[4-[2-(2,6-dioxo-3-piperidyl)-1-oxo-isoindolin-5-yl]piperazin-1- yl]-4-oxo-butyl]-1,2,4-oxadiazol-3-yl]-3-pyridyl]-3-(7-isopropylpyrazolo[1,5- a]pyrimidin-6-yl)urea |
| 141 | 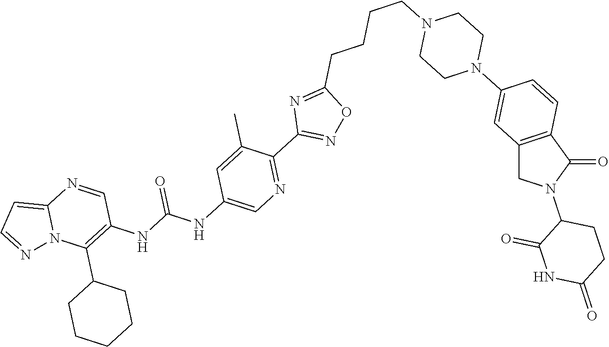 [SMILES: Cc1cc(NC(=O)Nc2cnc3ccnn3c2C2CCCCC2)cnc1-c1noc(CCCCN2CCN(c3ccc4c(c3)CN(C3CCC(=O)NC3=O)C4=O)CC2)n1] 1-(7-cyclohexylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butyl]-1,2,4-oxadiazol-3-yl]-5-methyl- 3-pyridyl]urea |
| 142 | 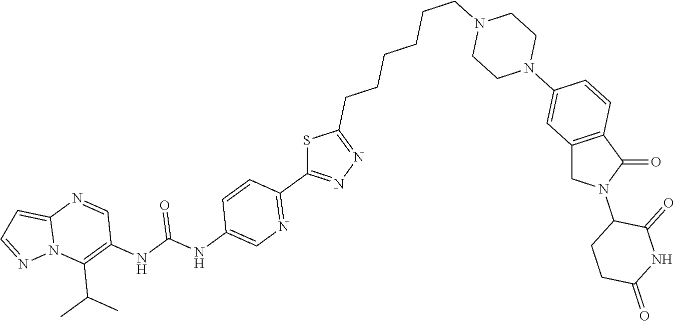 [SMILES: CC(C)c1c(NC(=O)Nc2ccc(-c3nnc(CCCCCCN4CCN(c5ccc6c(c5)CN(C5CCC(=O)NC5=O)C6=O)CC4)s3)nc2)cnc2ccnn12] 1-[6-[5-[6-[4-[2-(2,6-dioxo-3-piperidyl)-1-oxo-isoindolin-5-yl]piperazin-1-yl]hexyl]- 1,3,4-thiadiazol-2-yl]-3-pyridyl]-3-(7-isopropylpyrazolo[1,5-a]pyrimidin-6-yl)urea |
| 143 | 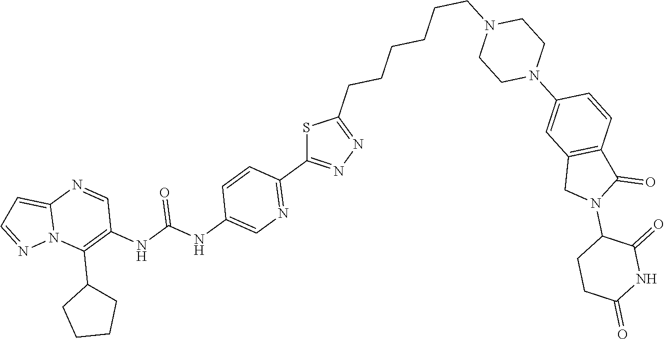 [SMILES: O=C1CCC(N2Cc3cc(N4CCN(CCCCCCc5nnc(-c6ccc(NC(=O)Nc7cnc8ccnn8c7C7CCCC7)cn6)s5)CC4)ccc3C2=O)C(=O)N1] 1-(7-cyclopentylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,3,4-thiadiazol-2-yl]-3- pyridyl]urea |
| 144 | 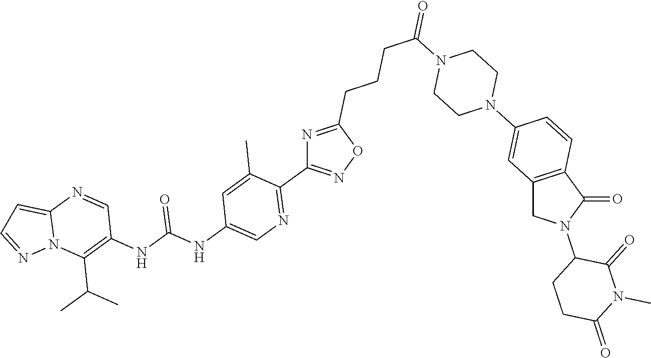 [SMILES: Cc1cc(NC(=O)Nc2cnc3ccnn3c2C(C)C)cnc1-c1noc(CCCC(=O)N2CCN(c3ccc4c(c3)CN(C3CCC(=O)N(C)C3=O)C4=O)CC2)n1] 1-(7-isopropylpyrazolo[1,5-a]pyrimidin-6-yl)-3-[5-methyl-6-[5-[4-[4-[2-(1-methyl-2,6- dioxo-3-piperidyl)-1-oxo-isoindolin-5-yl]piperazin-1-yl]-4-oxo-butyl]-1,2,4-oxadiazol- 3-yl]-3-pyridyl]urea |
| 145 | 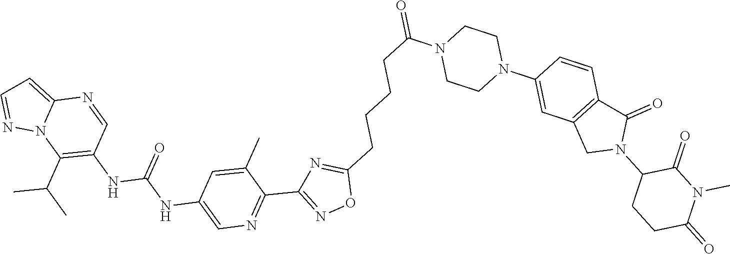 [SMILES: Cc1cc(NC(=O)Nc2cnc3ccnn3c2C(C)C)cnc1-c1noc(CCCCC(=O)N2CCN(c3ccc4c(c3)CN(C3CCC(=O)N(C)C3=O)C4=O)CC2)n1] 1-(7-isopropylpyrazolo[1,5-a]pyrimidin-6-yl)-3-[5-methyl-6-[5-[5-[4-[2-(1-methyl-2,6- dioxo-3-piperidyl)-1-oxo-isoindolin-5-yl]piperazin-1-yl]-5-oxo-pentyl]-1,2,4- oxadiazol-3-yl]-3-pyridyl]urea |
| 146 | 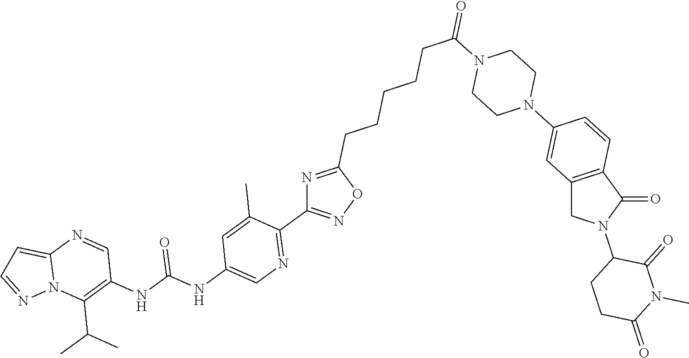 [SMILES: Cc1cc(NC(=O)Nc2cnc3ccnn3c2C(C)C)cnc1-c1noc(CCCCCC(=O)N2CCN(c3ccc4c(c3)CN(C3CCC(=O)N(C)C3=O)C4=O)CC2)n1] 1-(7-isopropylpyrazolo[1,5-a]pyrimidin-6-yl)-3-[5-methyl-6-[5-[6-[4-[2-(1-methyl-2,6- dioxo-3-piperidyl)-1-oxo-isoindolin-5-yl]piperazin-1-yl]-6-oxo-hexyl]-1,2,4- oxadiazol-3-yl]-3-pyridyl]urea |
| 147 | 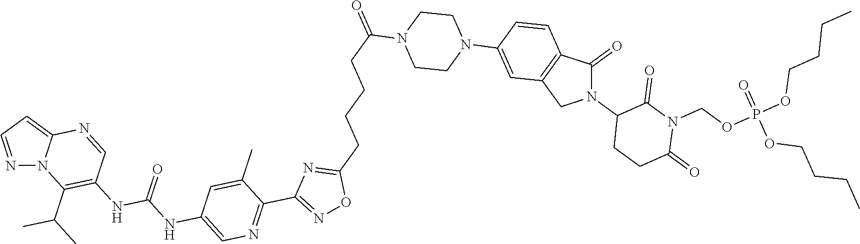 [SMILES: CCCCOP(=O)(OCCCC)OCN1C(=O)CCC(N2Cc3cc(N4CCN(C(=O)CCCCc5nc(-c6ncc(NC(=O)Nc7cnc8ccnn8c7C(C)C)cc6C)no5)CC4)ccc3C2=O)C1=O] dibutyl [3-[5-[4-[5-[3-[5-[(7-isopropylpyrazolo[1,5-a]pyrimidin-6- yl)carbamoylamino]-3-methyl-2-pyridyl]-1,2,4-oxadiazol-5-yl]pentanoyl]piperazin-1- yl]-1-oxo-isoindolin-2-yl]-2,6-dioxo-1-piperidyl]methyl phosphate |
| 148 | 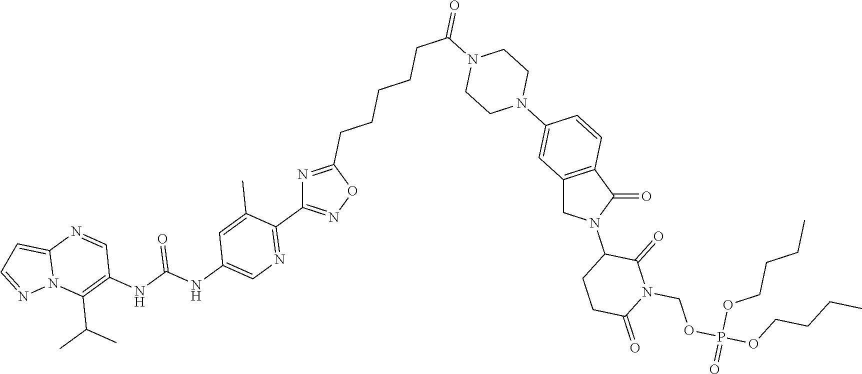 [SMILES: CCCCOP(=O)(OCCCC)OCN1C(=O)CCC(N2Cc3cc(N4CCN(C(=O)CCCCCc5nc(-c6ncc(NC(=O)Nc7cnc8ccnn8c7C(C)C)cc6C)no5)CC4)ccc3C2=O)C1=O] dibutyl [3-[5-[4-[6-[3-[5-[(7-isopropylpyrazolo[1,5-a]pyrimidin-6- yl)carbamoylamino]-3-methyl-2-pyridyl]-1,2,4-oxadiazol-5-yl]hexanoyl]piperazin-1- yl]-1-oxo-isoindolin-2-yl]-2,6-dioxo-1-piperidyl]methyl phosphate |
| 149 | 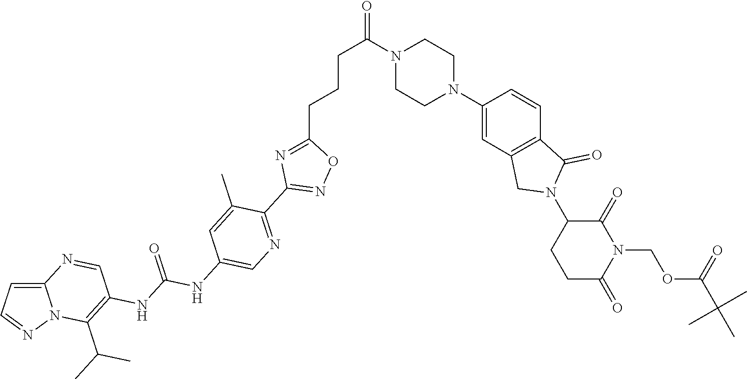 [SMILES: Cc1cc(NC(=O)Nc2cnc3ccnn3c2C(C)C)cnc1-c1noc(CCCC(=O)N2CCN(c3ccc4c(c3)CN(C3CCC(=O)N(COC(=O)C(C)(C)C)C3=O)C4=O)CC2)n1] [3-[5-[4-[4-[3-[5-[(7-isopropylpyrazolo[1,5-a]pyrimidin-6-yl)carbamoylamino]-3- methyl-2-pyridyl]-1,2,4-oxadiazol-5-yl]butanoyl]piperazin-1-yl]-1-oxo-isoindolin-2- yl]-2,6-dioxo-1-piperidyl]methyl 2,2-dimethylpropanoate |
| 150 | 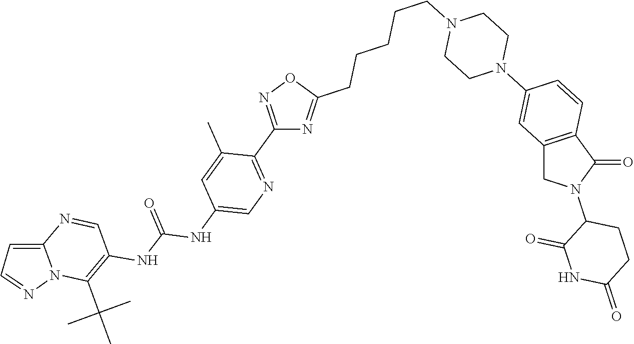 [SMILES: Cc1cc(NC(=O)Nc2cnc3ccnn3c2C(C)(C)C)cnc1-c1noc(CCCCCN2CCN(c3ccc4c(c3)CN(C3CCC(=O)NC3=O)C4=O)CC2)n1] 1-(7-tert-butylpyrazolo[1,5-a]pyrimidin-6-yl)-3-[6-[5-[5-[4-[2-(2,6-dioxo-3-piperidyl)- 1-oxo-isoindolin-5-yl]piperazin-1-yl]pentyl]-1,2,4-oxadiazol-3-yl]-5-methyl-3- pyridyl]urea |
| 151 | 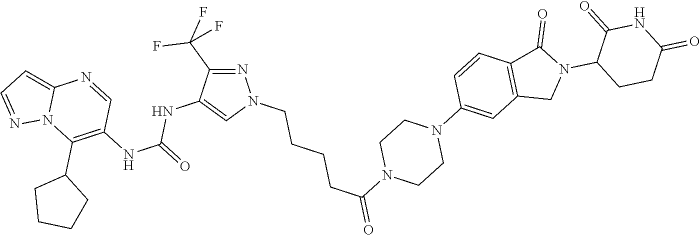 [SMILES: O=C1CCC(N2Cc3cc(N4CCN(C(=O)CCCCn5cc(NC(=O)Nc6cnc7ccnn7c6C6CCCC6)c(C(F)(F)F)n5)CC4)ccc3C2=O)C(=O)N1] 1-(7-cyclopentylpyrazolo[1,5-a]pyrimidin-6-yl)-3-[1-[5-[4-[2-(2,6-dioxo-3-piperidyl)- 1-oxo-isoindolin-5-yl]piperazin-1-yl]-5-oxo-pentyl]-3-(trifluoromethyl)pyrazol-4- yl]urea |
| 152 | 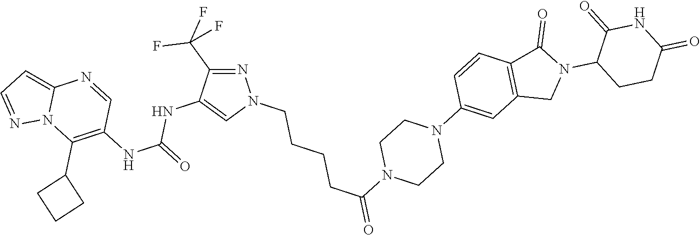 [SMILES: O=C1CCC(N2Cc3cc(N4CCN(C(=O)CCCCn5cc(NC(=O)Nc6cnc7ccnn7c6C6CCC6)c(C(F)(F)F)n5)CC4)ccc3C2=O)C(=O)N1] 1-(7-cyclobutylpyrazolo[1,5-a]pyrimidin-6-yl)-3-[1-[5-[4-[2-(2,6-dioxo-3-piperidyl)-1- oxo-isoindolin-5-yl]piperazin-1-yl]-5-oxo-pentyl]-3-(trifluoromethyl)pyrazol-4-yl]urea |
| 153 | 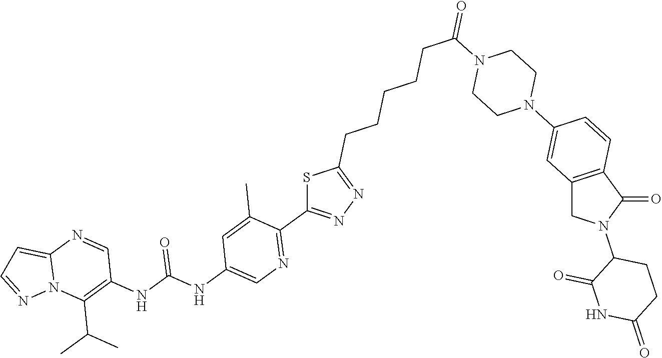 [SMILES: Cc1cc(NC(=O)Nc2cnc3ccnn3c2C(C)C)cnc1-c1nnc(CCCCCC(=O)N2CCN(c3ccc4c(c3)CN(C3CCC(=O)NC3=O)C4=O)CC2)s1] 1-[6-[5-[6-[4-[2-(2,6-dioxo-3-piperidyl)-1-oxo-isoindolin-5-yl]piperazin-1-yl]-6-oxo- hexyl]-1,3,4-thiadiazol-2-yl]-5-methyl-3-pyridyl]-3-(7-isopropylpyrazolo[1,5- a]pyrimidin-6-yl)urea |
| 154 | 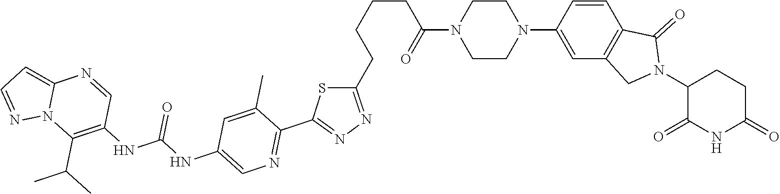 [SMILES: Cc1cc(NC(=O)Nc2cnc3ccnn3c2C(C)C)cnc1-c1nnc(CCCCC(=O)N2CCN(c3ccc4c(c3)CN(C3CCC(=O)NC3=O)C4=O)CC2)s1] 1-[6-[5-[5-[4-[2-(2,6-dioxo-3-piperidyl)-1-oxo-isoindolin-5-yl]piperazin-1-yl]-5-oxo- pentyl]-1,3,4-thiadiazol-2-yl]-5-methyl-3-pyridyl]-3-(7-isopropylpyrazolo[1,5- a]pyrimidin-6-yl)urea |
| 155 | 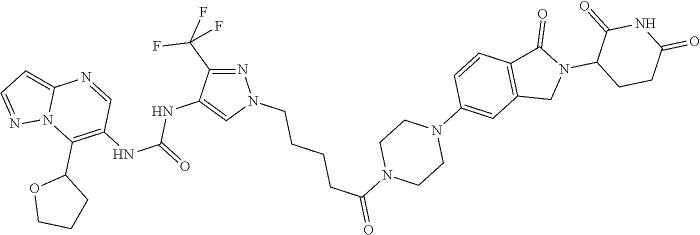 [SMILES: O=C1CCC(N2Cc3cc(N4CCN(C(=O)CCCCn5cc(NC(=O)Nc6cnc7ccnn7c6C6CCCO6)c(C(F)(F)F)n5)CC4)ccc3C2=O)C(=O)N1] 1-[1-[5-[4-[2-(2,6-dioxo-3-piperidyl)-1-oxo-isoindolin-5-yl]piperazin-1-yl]-5-oxo- pentyl]-3-(trifluoromethyl)pyrazol-4-yl]-3-(7-tetrahydrofuran-2-ylpyrazolo[1,5- a]pyrimidin-6-yl)urea |
| 156 | 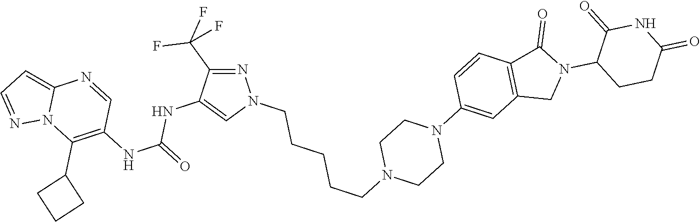 [SMILES: O=C1CCC(N2Cc3cc(N4CCN(CCCCCn5cc(NC(=O)Nc6cnc7ccnn7c6C6CCC6)c(C(F)(F)F)n5)CC4)ccc3C2=O)C(=O)N1] 1-(7-cyclobutylpyrazolo[1,5-a]pyrimidin-6-yl)-3-[1-[5-[4-[2-(2,6-dioxo-3-piperidyl)-1- oxo-isoindolin-5-yl]piperazin-1-yl]pentyl]-3-(trifluoromethyl)pyrazol-4-yl]urea |
| 157 | 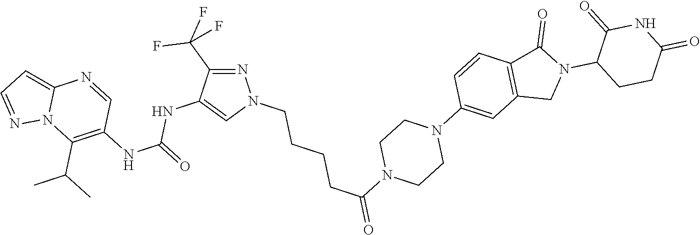 [SMILES: CC(C)c1c(NC(=O)Nc2cn(CCCCC(=O)N3CCN(c4ccc5c(c4)CN(C4CCC(=O)NC4=O)C5=O)CC3)nc2C(F)(F)F)cnc2ccnn12] 1-[1-[5-[4-[2-(2,6-dioxo-3-piperidyl)-1-oxo-isoindolin-5-yl]piperazin-1-yl]-5-oxo- pentyl]-3-(trifluoromethyl)pyrazol-4-yl]-3-(7-isopropylpyrazolo[1,5-a]pyrimidin-6- yl)urea |
| 158 | 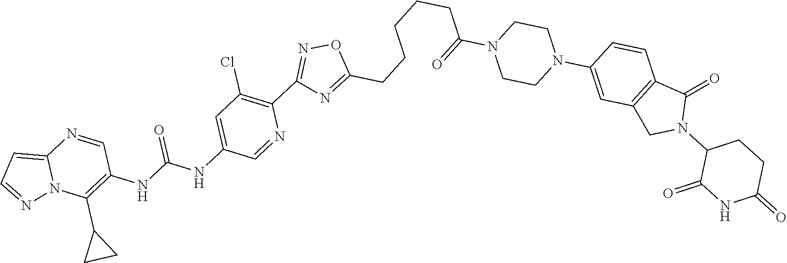 [SMILES: O=C1CCC(N2Cc3cc(N4CCN(C(=O)CCCCCc5nc(-c6ncc(NC(=O)Nc7cnc8ccnn8c7C7CC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]-6-oxo-hexyl]-1,2,4-oxadiazol-3-yl]-3-pyridyl]-3-(7-cyclopropylpyrazolo[1,5- a]pyrimidin-6-yl)urea |
| 159 | 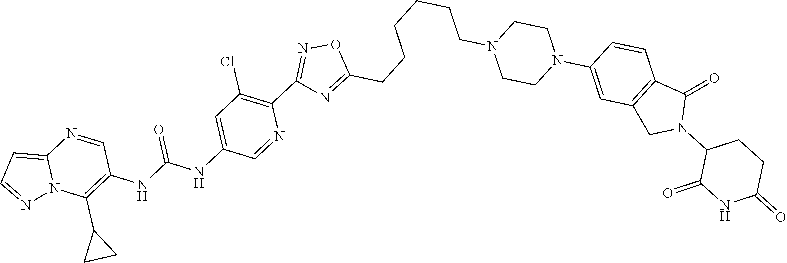 [SMILES: O=C1CCC(N2Cc3cc(N4CCN(CCCCCCc5nc(-c6ncc(NC(=O)Nc7cnc8ccnn8c7C7CC7)cc6Cl)no5)CC4)ccc3C2=O)C(=O)N1] 1-[5-chloro-6-[5-[6-[4-[2-(2,6-dioxo-3-piperidyl)-1-oxo-isoindolin-5-yl]piperazin-1- yl]hexyl]-1,2,4-oxadiazol-3-yl]-3-pyridyl]-3-(7-cyclopropylpyrazolo[1,5-a]pyrimidin- 6-yl)urea |
| 160 | 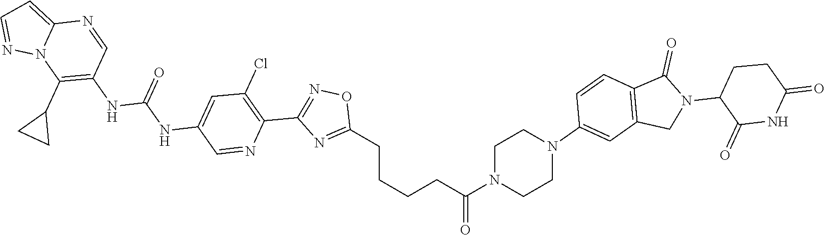 [SMILES: O=C1CCC(N2Cc3cc(N4CCN(C(=O)CCCCc5nc(-c6ncc(NC(=O)Nc7cnc8ccnn8c7C7CC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]-5-oxo-pentyl]-1,2,4-oxadiazol-3-yl]-3-pyridyl]-3-(7-cyclopropylpyrazolo[1,5- a]pyrimidin-6-yl)urea |
| 161 | 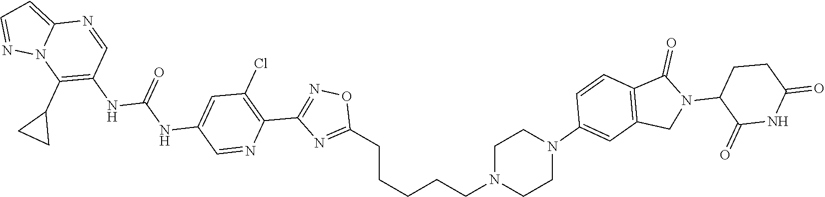 [SMILES: O=C1CCC(N2Cc3cc(N4CCN(CCCCCc5nc(-c6ncc(NC(=O)Nc7cnc8ccnn8c7C7CC7)cc6Cl)no5)CC4)ccc3C2=O)C(=O)N1] 1-[5-chloro-6-[5-[5-[4-[2-(2,6-dioxo-3-piperidyl)-1-oxo-isoindolin-5-yl]piperazin-1- yl]pentyl]-1,2,4-oxadiazol-3-yl]-3-pyridyl]-3-(7-cyclopropylpyrazolo[1,5-a]pyrimidin- 6-yl)urea |
| 162 | 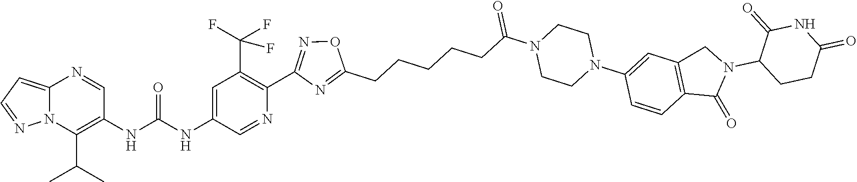 [SMILES: CC(C)c1c(NC(=O)Nc2cnc(-c3noc(CCCCCC(=O)N4CCN(c5ccc6c(c5)CN(C5CCC(=O)NC5=O)C6=O)CC4)n3)c(C(F)(F)F)c2)cnc2ccnn12] 1-[6-[5-[6-[4-[2-(2,6-dioxo-3-piperidyl)-1-oxo-isoindolin-5-yl]piperazin-1-yl]-6-oxo- hexyl]-1,2,4-oxadiazol-3-yl]-5-(trifluoromethyl)-3-pyridyl]-3-(7- isopropylpyrazolo[1,5-a]pyrimidin-6-yl)urea |
| 163 | 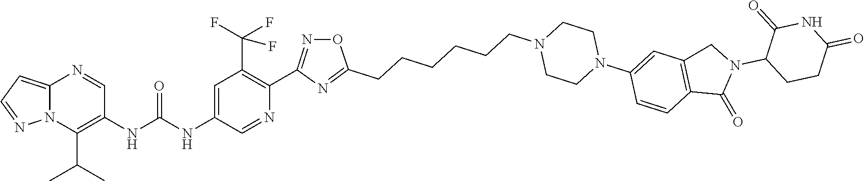 [SMILES: CC(C)c1c(NC(=O)Nc2cnc(-c3noc(CCCCCCN4CCN(c5ccc6c(c5)CN(C5CCC(=O)NC5=O)C6=O)CC4)n3)c(C(F)(F)F)c2)cnc2ccnn12] 1-[6-[5-[6-[4-[2-(2,6-dioxo-3-piperidyl)-1-oxo-isoindolin-5-yl]piperazin-1-yl]hexyl]- 1,2,4-oxadiazol-3-yl]-5-(trifluoromethyl)-3-pyridyl]-3-(7-isopropylpyrazolo[1,5- a]pyrimidin-6-yl)urea |
| 164 | 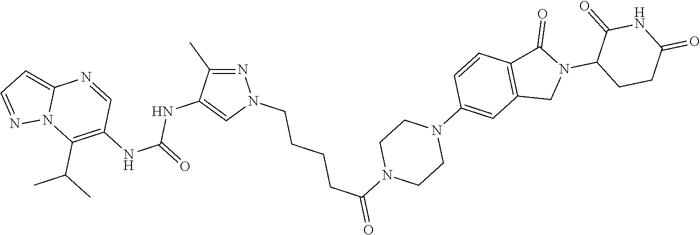 [SMILES: Cc1nn(CCCCC(=O)N2CCN(c3ccc4c(c3)CN(C3CCC(=O)NC3=O)C4=O)CC2)cc1NC(=O)Nc1cnc2ccnn2c1C(C)C] 1-[1-[5-[4-[2-(2,6-dioxo-3-piperidyl)-1-oxo-isoindolin-5-yl]piperazin-1-yl]-5-oxo- pentyl]-3-methyl-pyrazol-4-yl]-3-(7-isopropylpyrazolo[1,5-a]pyrimidin-6-yl)urea |
| 165 | 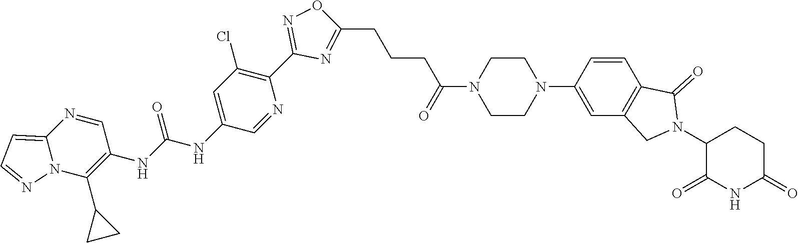 [SMILES: O=C1CCC(N2Cc3cc(N4CCN(C(=O)CCCc5nc(-c6ncc(NC(=O)Nc7cnc8ccnn8c7C7CC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]-4-oxo-butyl]-1,2,4-oxadiazol-3-yl]-3-pyridyl]-3-(7-cyclopropylpyrazolo[1,5- a]pyrimidin-6-yl)urea |
| 166 | 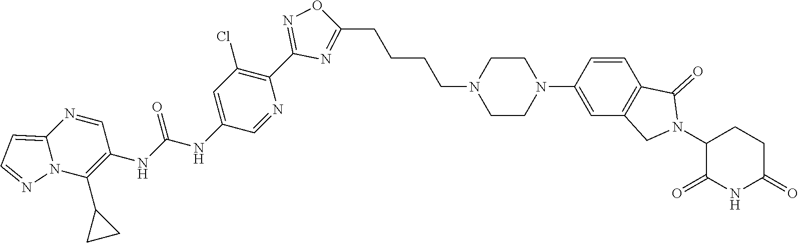 [SMILES: O=C1CCC(N2Cc3cc(N4CCN(CCCCc5nc(-c6ncc(NC(=O)Nc7cnc8ccnn8c7C7CC7)cc6Cl)no5)CC4)ccc3C2=O)C(=O)N1] 1-[5-chloro-6-[5-[4-[4-[2-(2,6-dioxo-3-piperidyl)-1-oxo-isoindolin-5-yl]piperazin-1- yl]butyl]-1,2,4-oxadiazol-3-yl]-3-pyridyl]-3-(7-cyclopropylpyrazolo[1,5-a]pyrimidin- 6-yl)urea |
| 167 | 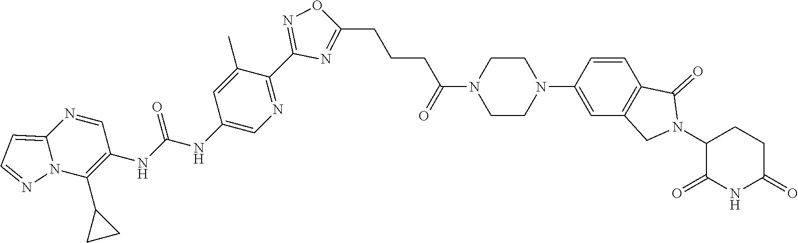 [SMILES: Cc1cc(NC(=O)Nc2cnc3ccnn3c2C2CC2)cnc1-c1noc(CCCC(=O)N2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]-6-oxo-hexyl]-1,2,4-oxadiazol-3-yl]-5- methyl-3-pyridyl]urea |
| 168 | 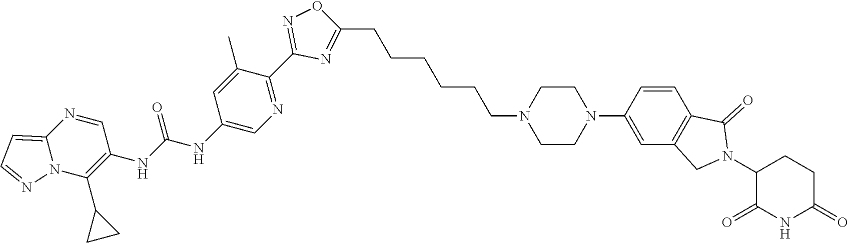 [SMILES: Cc1cc(NC(=O)Nc2cnc3ccnn3c2C2CC2)cnc1-c1noc(CCCCCCN2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[6-[4-[2-(2,6-dioxo-3- piperidyl)-1-oxo-isoindolin-5-yl]piperazin-1-yl]hexyl]-1,2,4-oxadiazol-3-yl]-5-methyl- 3-pyridyl]urea |
| 169 | 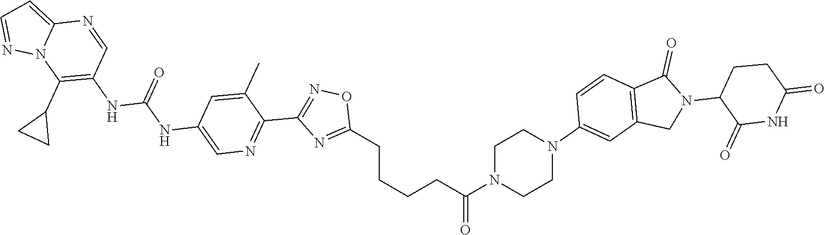 [SMILES: Cc1cc(NC(=O)Nc2cnc3ccnn3c2C2CC2)cnc1-c1noc(CCCCC(=O)N2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]-5-oxo-pentyl]-1,2,4-oxadiazol-3-yl]-5- methyl-3-pyridyl]urea |
| 170 | 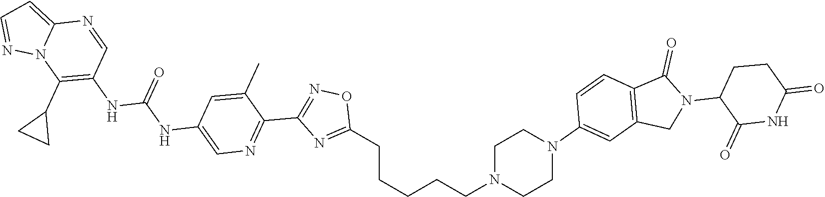 [SMILES: Cc1cc(NC(=O)Nc2cnc3ccnn3c2C2CC2)cnc1-c1noc(CCCCCN2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[5-[4-[2-(2,6-dioxo-3- piperidyl)-1-oxo-isoindolin-5-yl]piperazin-1-yl]pentyl]-1,2,4-oxadiazol-3-yl]-5- methyl-3-pyridyl]urea |
| 171 | 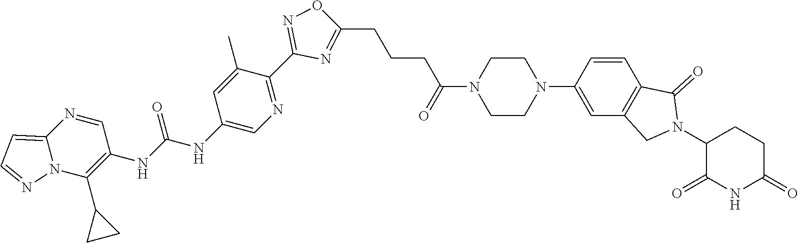 [SMILES: Cc1cc(NC(=O)Nc2cnc3ccnn3c2C2CC2)cnc1-c1noc(CCCC(=O)N2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]-4-oxo-butyl]-1,2,4-oxadiazol-3-yl]-5- methyl-3-pyridyl]urea |
| 172 | 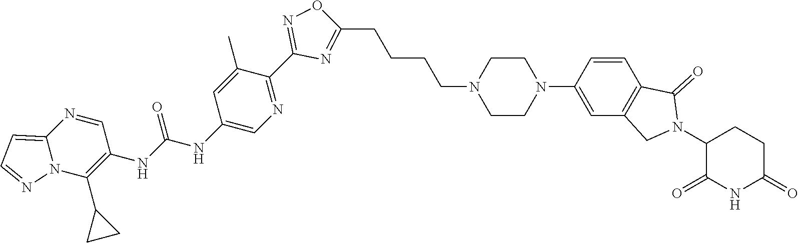 [SMILES: Cc1cc(NC(=O)Nc2cnc3ccnn3c2C2CC2)cnc1-c1noc(CCCCN2CCN(c3ccc4c(c3)CN(C3CCC(=O)NC3=O)C4=O)CC2)n1] 1-(7-cyclopropylpyrazolo[1,5-a]pyrimidin-6-yl)-3-[6-[5-[4-[4-[2-(2,6-dioxo-3- piperidyl)-1-oxo-isoindolin-5-yl]piperazin-1-yl]butyl]-1,2,4-oxadiazol-3-yl]-5-methyl- 3-pyridyl]urea |
| 173 | 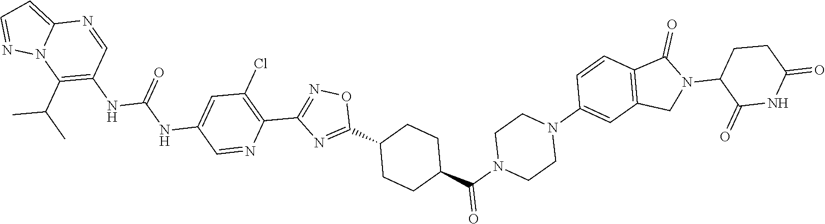 [SMILES: CC(C)c1c(NC(=O)Nc2cnc(-c3noc([C@H]4CC[C@H](C(=O)N5CCN(c6ccc7c(c6)CN(C6CCC(=O)NC6=O)C7=O)CC5)CC4)n3)c(Cl)c2)cnc2ccnn12] 1-[5-chloro-6-[5-[4-[4-[2-(2,6-dioxo-3-piperidyl)-1-oxo-isoindolin-5-yl]piperazine-1- carbonyl]cyclohexyl]-1,2,4-oxadiazol-3-yl]-3-pyridyl]-3-(7-isopropylpyrazolo[1,5- a]pyrimidin-6-yl)urea |
| 174 | 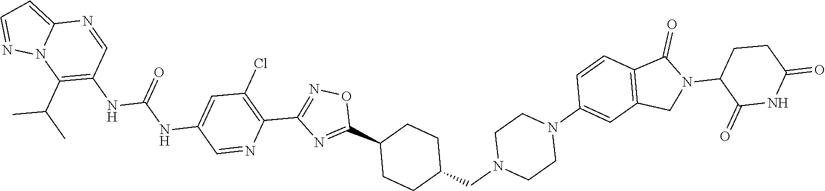 [SMILES: CC(C)c1c(NC(=O)Nc2cnc(-c3noc([C@H]4CC[C@H](CN5CCN(c6ccc7c(c6)CN(C6CCC(=O)NC6=O)C7=O)CC5)CC4)n3)c(Cl)c2)cnc2ccnn12] 1-[5-chloro-6-[5-[4-[[4-[2-(2,6-dioxo-3-piperidyl)-1-oxo-isoindolin-5-yl]piperazin-1- yl]methyl]cyclohexyl]-1,2,4-oxadiazol-3-yl]-3-pyridyl]-3-(7-isopropylpyrazolo[1,5- a]pyrimidin-6-yl)urea |
| 175 | 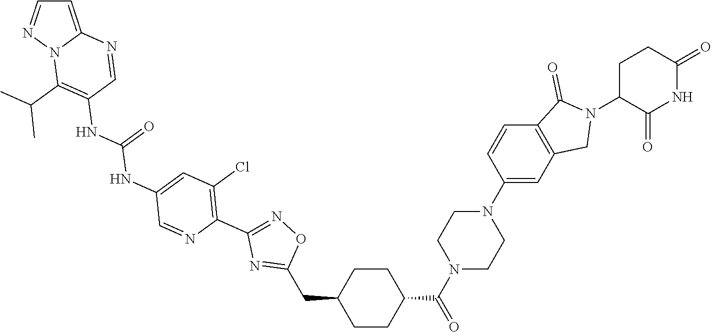 [SMILES: CC(C)c1c(NC(=O)Nc2cnc(-c3noc(C[C@H]4CC[C@H](C(=O)N5CCN(c6ccc7c(c6)CN(C6CCC(=O)NC6=O)C7=O)CC5)CC4)n3)c(Cl)c2)cnc2ccnn12] 1-[5-chloro-6-[5-[[4-[4-[2-(2,6-dioxo-3-piperidyl)-1-oxo-isoindolin-5-yl]piperazine-1- carbonyl]cyclohexyl]methyl]-1,2,4-oxadiazol-3-yl]-3-pyridyl]-3-(7- isopropylpyrazolo[1,5-a]pyrimidin-6-yl)urea |
| 176 | 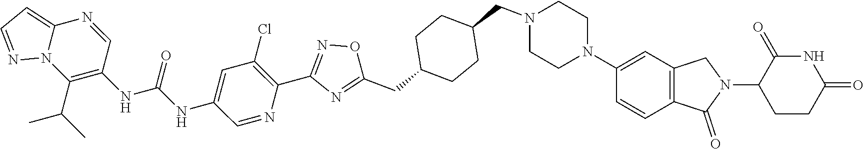 [SMILES: CC(C)c1c(NC(=O)Nc2cnc(-c3noc(C[C@H]4CC[C@H](CN5CCN(c6ccc7c(c6)CN(C6CCC(=O)NC6=O)C7=O)CC5)CC4)n3)c(Cl)c2)cnc2ccnn12] 1-[5-chloro-6-[5-[[4-[[4-[2-(2,6-dioxo-3-piperidyl)-1-oxo-isoindolin-5-yl]piperazin-1- yl]methyl]cyclohexyl]methyl]-1,2,4-oxadiazol-3-yl]-3-pyridyl]-3-(7- isopropylpyrazolo[1,5-a]pyrimidin-6-yl)urea |

or a pharmaceutically acceptable salt, stereoisomer, solvate, or tautomer thereof.

19. A pharmaceutical composition comprising the compound or pharmaceutically acceptable salt, solvate, hydrate, stereoisomer, or tautomer thereof of claim 1, and a pharmaceutically acceptable carrier.

20. The pharmaceutical composition of claim 19, further comprising an additional pharmaceutically active agent.

21. A method of degrading a MALT1, comprising of administering to a subject a compound of claim 1.

22. A method of treating a disease or disorder associated with MALT1, comprising of administering to a subject a compound of claim 1.

23. A method of treating disease or disorder selected from Lymphoma and Immunodeficiency, comprising of administering to a subject in need of a treatment a compound of claim 1.

24. The method of any one of claims 21-23, wherein the subject is a mammal.

25. The method of claim 24, wherein the subject is a human.
